(19) United States
(12) Patent Application Publication
(10) Pub. No.: US 2019/0053492 A1
(43) Pub. Date: Feb. 21, 2019

(54) SUBSTITUTED PYRIDINE COMPOUNDS AS PESTICIDES

(71) Applicant: Bayer CropScience Aktiengesellschaft, Monheim am Rhein (DE)

(72) Inventors: David WILCKE, Düsseldorf (DE); Roland ANDREE, Langenfeld (DE); Arnd VOERSTE, Köln (DE); Silvia CEREZO-GALVEZ, Langenfeld (DE); Graham HOLMWOOD, Leverkusen (DE); Kerstin ILG, Köln (DE); Daniela PORTZ, Vettweiss (DE); Ulrich EBBINGHAUS-KINTSCHER, Dortmund (DE); Ulrich GÖRGENS, Ratingen (DE); Cathleen BRADLER, Köln (DE); Andreas TURBERG, Haan (DE)

(21) Appl. No.: 16/084,655

(22) PCT Filed: Mar. 14, 2017
(86) PCT No.: PCT/EP2017/055902

§ 371 Date: Sep. 13, 2018

(30) Foreign Application Priority Data
Mar. 16, 2016 (EP) 16160593.6

(51) Int. Cl. A01N 47/12; A01N 43/40; A01N 53/00; C07D 213/81

(52) U.S. Cl. CPC A01N 47/12; A01N 43/40; A01N 25/04; C07D 213/81; A01N 53/00

(57) ABSTRACT

The present invention relates to novel substituted pyridine compounds of the formula (I)

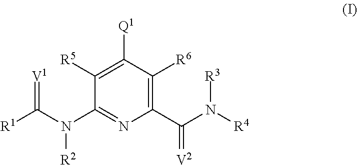

in which R¹, R², R³, R⁴, R⁵, R⁶, V¹, V² and Q¹ have the definitions given in the description, and to the use thereof as acaricides and insecticides for control of animal pests, particularly arthropods and especially insects and arachnids, and to processes and intermediates for preparation thereof.

The present invention relates to substituted pyridine compounds of the formula (I), to the use thereof as acaricides and/or insecticides for controlling animal pests, particularly arthropods and especially insects and arachnids, and to processes and intermediates for the preparation thereof.

WO 2012/054510 A1 describes 4-oxadiazole-substituted pyridines as inhibitors of beta-secretase activity, which can especially be used for the treatment of Alzheimer's disease.

WO 2012/069366 A1 discloses, inter alia, in table F, insecticidally active compounds containing a pyridinyl group, and containing a fluoroalkyl-substituted pyrazole group (Q2) bonded to the pyridinyl group via the —NH—CO— moiety.

The compounds according to the invention, by contrast, contain a Q1 substituent in position 4 on the pyridine ring, which is necessarily optionally substituted aryl, 1,3-benzodioxolyl, 2,3-dihydro-1,4-benzodioxinyl, hetaryl or oxohetaryl. However, no such substituent is present in the compounds disclosed in WO 2012/069366 A1.

Modern crop protection compositions have to meet many demands, for example in relation to the level, duration and spectrum of their action and possible use. Questions of toxicity and of combinability with other active ingredients or formulation auxiliaries play a role, as does the question of the cost and complexity involved in the synthesis of an active ingredient. In addition, resistances can occur. For all these reasons alone, the search for novel crop protection compositions cannot be considered complete, and there is a constant need for novel compounds having improved properties compared to the known compounds, at least in relation to individual aspects.

It was an object of the present invention to provide compounds which broaden the spectrum of the pesticides in various aspects and/or improve their activity.

It has now been found that, surprisingly, particular novel substituted pyridine compounds of the formula (I) and salts thereof have significant insecticidal and acaricidal properties with simultaneously good plant tolerance, favourable homeotherm toxicity and good environmental compatibility.

The present invention therefore provides (Configuration 1-1) compounds of the general formula (I)

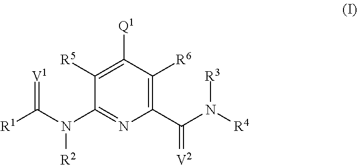

in which
Q¹ is in each case optionally singly or multiply, identically or differently substituted aryl, 1,3-benzodioxolyl, 2,3-dihydro-1,4-benzodioxinyl, hetaryl or oxohetaryl,
   where the substituents are selected from: cyano, carboxyl, halogen, nitro, hydroxyl, amino, SCN, SF₅, tri-(C₁-C₆)alkylsilyl, (C₁-C₆)alkyl, (C₁-C₆)haloalkyl, (C₁-C₆)cyanoalkyl, (C₁-C₆)hydroxyalkyl, hydroxycarbonyl-(C₁-C₆)-alkoxy, (C₁-C₆)alkoxycarbonyl-(C₁-C₆)alkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁-C₆)alkoxycarbonyl-(C₁-C₆)alkoxy, (C₁-C₆)alkoxy-(C₁-C₆)alkoxy, (C₃-C₈)cycloalkyl, (C₃-C₈)halocycloalkyl, (C₁-C₆)alkyl-(C₃-C₈)cycloalkyl, (C₁-C₆)haloalkyl-(C₃-C₈)cycloalkyl, (C₁-C₄)haloalkenoxy, (C₁-C₆)alkylhydroxyimino, (C₁-C₆)alkoxyimino, (C₁-C₆)alkyl-(C₁-C₆)alkoxyimino, (C₁-C₆)haloalkyl-(C₁-C₆)alkoxyimino, (C₁-C₆)alkylsulphanyl, (C₁-C₆)alkoxy-(C₁-C₆)alkylsulphanyl, (C₁-C₆)alkylsulphanyl-(C₁-C₆)alkyl, (C₁-C₆)alkylsulphinyl, (C₁-C₆)haloalkylsulphinyl, (C₁-C₆)alkoxy-(C₁-C₆)alkylsulphinyl, (C₁-C₆)alkylsulphinyl-(C₁-C₆)alkyl, (C₁-C₆)alkylsulphonyl, (C₁-C₆)haloalkylsulphonyl, (C₁-C₆)alkoxy-(C₁-C₆)alkylsulphonyl, (C₁-C₆)alkylsulphonyl-(C₁-C₆)alkyl, (C₁-C₆)alkylsulphonyloxy, (C₁-C₆)haloalkylsulphanyl, (C₃-C₆)cycloalkylsulphanyl, (C₃-C₆)halocycloalkylsulphanyl, (C₃-C₆)cycloalkylsulphonyl, (C₃-C₆)halocycloalkylsulphonyl, (C₃-C₆)cycloalkylsulphinyl, (C₃-C₆)halocycloalkylsulphinyl, (C₁-C₆)alkylcarbonyl, (C₁-C₆)haloalkylcarbonyl, (C₁-C₆)alkylcarbonyloxy, (C₁-C₆)alkoxycarbonyl, (C₁-C₆)haloalkoxycarbonyl, aminocarbonyl, (C₁-C₆)alkylaminocarbonyl, di-(C₁-C₆)alkylaminocarbonyl, (C₂-C₆)alkenylaminocarbonyl, di-(C₂-C₆)-alkenylaminocarbonyl, (C₃-C₈)cycloalkylaminocarbonyl, (C₁-C₆)alkylsulphonylamino, (C₁-C₆)alkylamino, di-(C₁-C₆)alkylamino, (C₁-C₆)haloalkylamino, bis-(C₁-C₆)haloalkylamino, aminosulphonyl, (C₁-C₆)alkylaminosulphonyl, di-(C₁-C₆)alkylaminosulphonyl, (C₁-C₆)alkylsulphoximino, aminothiocarbonyl, (C₁-C₆)alkylaminothiocarbonyl, di-(C₁-C₆)alkylaminothiocarbonyl, (C₃-C₈)cycloalkylamino,
R¹ is (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl, (C₃-C₈)cycloalkyl-(C₁-C₆)alkyl, (C₃-C₈)cycloalkyl-(C₃-C₈)cycloalkyl, (C₁-C₆)alkyl-(C₃-C₈)cycloalkyl, (C₁-C₆)haloalkyl-(C₃-C₈)cycloalkyl, halo(C₃-C₈)cycloalkyl, cyano(C₃-C₈)cycloalkyl, (C₂-C₆)alkynyl-(C₃-C₈)cycloalkyl, (C₁-C₆)alkoxy-(C₃-C₈)cycloalkyl, (C₁-C₆)alkoxycarbonyl-(C₃-C₈)cycloalkyl, (C₁-C₆)alkylamino, di-(C₁-C₆)alkylamino, (C₃-C₈)cycloalkylamino, (C₁-C₆)alkylcarbonylamino, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, (C₁-C₆)alkylsulphonylamino, aminosulphonyl-(C₁-C₆)alkyl, (C₁-C₆)alkylaminosulphonyl-(C₁-C₆)alkyl, di-(C₁-C₆)alkylaminosulphonyl-(C₁-C₆)alkyl, (C₁-C₄)alkoxy, (C₃-C₆)cycloalkoxy, (C₃-C₆)cycloalkylcarbonyl or (C₁-C₄)alkylcarbonyl,
   or in each case optionally singly or multiply, identically or differently aryl-, hetaryl-, oxohetaryl-, heterocyclyl- or oxoheterocyclyl-substituted (C₁-C₆)alkyl, (C₁-C₆)alkoxy, (C₂-C₆)alkenyl, (C₂-C₆)alkynyl, (C₃-C₈)cycloalkyl, where aryl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, aminosulphonyl, (C₁-C₆)alkyl, (C₃-C₆)cycloalkyl, (C₁-C₆)alkoxy, (C₁-C₆)haloalkyl, (C₁-C₆)haloalkoxy, (C₁-C₆)alkylthio, (C₁-C₆)alkylsulphinyl, (C₁-C₆)alkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl or (C₃-C₆)trialkylsilyl,
   or in each case optionally singly or multiply, identically or differently substituted aryl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₈)cycloalkyl, (C₁-C₆)-alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl,
R² is hydrogen (C₁-C₆)alkyl, (C₃-C₈)cycloalkyl, (C₁-C₆)alkoxy, (C₁-C₆)alkylcarbonyl, (C₃-C₈)cycloalkylcarbonyl, (C₁-C₆)alkoxycarbonyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl-(C₃-C₈)cycloalkyl, (C₁-C₆)alkyl-(C₃-C₈)cycloalkyl, halo(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, aminocarbonyl-(C₁-C₆)alkyl, (C₁-C₆)alkylamino-(C₁-C₆)alkyl, di-(C₁-C₆)alkylamino-(C₁-C₆)alkyl or (C₃-C₈)cycloalkylamino-(C₁-C₆)alkyl,
R³ is hydrogen (C₁-C₆)alkyl, (C₃-C₈)cycloalkyl, (C₁-C₆)alkoxy, (C₁-C₆)alkylcarbonyl, (C₃-C₈)cycloalkylcarbonyl, (C₁-C₆)alkoxycarbonyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl-(C₃-C₈)cycloalkyl, (C₁-C₆)alkyl-(C₃-C₈)cycloalkyl, halo(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, aminocarbonyl-(C₁-C₆)alkyl, (C₁-C₆)alkylamino-(C₁-C₆)alkyl, di-(C₁-C₆)alkylamino-(C₁-C₆)alkyl or (C₃-C₈)cycloalkylamino-(C₁-C₆)alkyl,
R⁴ is (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl, (C₃-C₈)cycloalkyl-(C₁-C₆)alkyl, cyano(C₃-C₈)cycloalkyl-(C₁-C₆)alkyl, halo(C₃-C₈)cycloalkyl-(C₁-C₆)alkyl, (C₁-C₄)haloalkyl-(C₃-C₈)cycloalkyl-(C₁-C₆)alkyl, (C₃-C₈)cycloalkyl-(C₃-C₈)cycloalkyl, (C₁-C₆)alkyl-(C₃-C₈)cycloalkyl, (C₁-C₆)haloalkyl-(C₃-C₈)cycloalkyl, halo(C₃-C₈)cycloalkyl, cyano(C₃-C₈)cycloalkyl, (C₂-C₆)alkynyl-(C₃-C₈)cycloalkyl, (C₁-C₆)alkoxy-(C₃-C₈)cycloalkyl, (C₁-C₆)alkoxycarbonyl-(C₃-C₈)cycloalkyl, carbamoyl-(C₃-C₈)cycloalkyl, thiocarbamoyl-(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, (C₁-C₆)alkylsulphonylamino, aminosulphonyl-(C₁-C₆)alkyl, (C₁-C₆)alkylaminosulphonyl-(C₁-C₆)alkyl, di-(C₁-C₆)alkylaminosulphonyl-(C₁-C₆)alkyl, amino, (C₁-C₆)alkylamino, di-(C₁-C₆)alkylamino, (C₃-C₈)cycloalkylamino, N—(C₁-C₆)alkyl-(C₃-C₈)cycloalkylamino, benzylamino, cyanobenzylamino, nitrobenzylamino, halobenzylamino, N—(C₁-C₆)alkylbenzylamino, N—(C₁-C₆)alkylcyanobenzylamino, N—(C₁-C₆)alkylnitrobenzylamino, N—(C₁-C₆)alkylhalobenzylamino, (C₁-C₆)alkylcarbonylamino, (C₃-C₈)cycloalkylcarbonylamino, hydroxyl, (C₁-C₆)alkoxy, (C₃-C₈)cycloalkoxy, (C₃-C₈)cycloalkyl-(C₁-C₆)alkoxy, benzyloxy, cyanobenzyloxy, nitrobenzyloxy, halobenzyloxy, (C₁-C₆)alkylimino, (C₃-C₈)cycloalkylimino, benzylimino, cyanobenzylimino, nitrobenzylimino or halobenzylimino,
   or in each case optionally singly or multiply, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₆)alkyl, (C₁-C₆)haloalkyl, (C₁-C₆)alkoxy, (C₂-C₆)alkenyl, (C₂-C₆)alkynyl, (C₃-C₈)cycloalkyl, oxy, amino, N—(C₁-C₆)alkylamino or N—(C₃-C₈)cycloalkylamino, where aryl, 1,3-benzodioxolyl, 2,3-dihydro-1,4-benzodioxinyl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, thiocarbamoyl, aminosulphonyl, (C₁-C₆)alkyl, (C₃-C₆)cycloalkyl, (C₁-C₆)alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)haloalkylsulphinyl, (C₁-C₆)haloalkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl or hetaryl, or
   in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₈)cycloalkyl, (C₁-C₆)-alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)haloalkylsulphinyl, (C₁-C₆)haloalkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl,
      or R³ and R⁴ together are (C₂-C₆)-alkyl or (C₂-C₆)alkenyl, forming a 3-7-membered, optionally halogen-, cyano-, hydroxyl-, amino-, carboxyl-, carbamoyl-, (C₁-C₆)alkyl-, (C₃-C₈)cycloalkyl-, (C₁-C₆)-alkoxy-, (C₁-C₆)haloalkyl-, (C₁-C₆)haloalkoxy-, (C₁-C₆)alkylthio- or (C₁-C₆)haloalkylthio-substituted ring which may optionally contain one to two double bonds,
R⁵, R⁶ are independently hydrogen, cyano, halogen, nitro, acetyl, hydroxyl, amino, (C₁-C₆)alkylamino, di-(C₁-C₆)alkylamino, (C₃-C₆)cycloalkyl, halo(C₃-C₆)cycloalkyl, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₁-C₆)alkoxy, (C₁-C₆)haloalkoxy, (C₁-C₆)alkylthio, (C₁-C₆)haloalkylthio, (C₁-C₆)alkylsulphinyl, (C₁-C₆)haloalkylsulphinyl, (C₁-C₆)alkylsulphonyl, (C₁-C₆)haloalkylsulphonyl,
V¹, V² are independently oxygen or sulphur.

Alternative compounds that are likewise part of the present invention (Configuration 1-2) are those of the formula (I) in which
Q¹ is in each case optionally singly or multiply, identically or differently substituted aryl, 1,3-benzodioxolyl, 2,3-dihydro-1,4-benzodioxinyl, hetaryl or oxohetaryl,
   where the substituents are selected from: cyano, carboxyl, halogen, nitro, hydroxyl, amino, SCN, SF₅, tri-(C₁-C₆)alkylsilyl, (C₁-C₆)alkyl, (C₁-C₆)haloalkyl, (C₁-C₆)cyanoalkyl, (C₁-C₆)hydroxyalkyl, hydroxycarbonyl-(C₁-C₆)-alkoxy, (C₁-C₆)alkoxycarbonyl-(C₁-C₆)alkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁-C₆)alkoxycarbonyl-(C₁-C₆)alkoxy, (C₁-C₆)alkoxy-(C₁-C₆)alkoxy, (C₃-C₈)cycloalkyl, (C₃-C₈)halocycloalkyl, (C₁-C₆)alkyl-(C₃-C₈)cycloalkyl, (C₁-C₆)haloalkyl-(C₃-C₈)cycloalkyl, (C₁-C₄)haloalkenoxy, (C₁-C₆)alkylhydroxyimino, (C₁-C₆)alkoxyimino, (C₁-C₆)alkyl-(C₁-C₆)alkoxyimino, (C₁-C₆)haloalkyl-(C₁-C₆)alkoxyimino, (C₁-C₆)alkylsulphanyl, (C₁-C₆)alkoxy-(C₁-C₆)alkylsulphanyl, (C₁-C₆)alkylsulphanyl-(C₁-C₆)alkyl, (C₁-C₆)alkylsulphinyl, (C₁-C₆)haloalkylsulphinyl, (C₁-C₆)alkoxy-(C₁-C₆)alkylsulphinyl, (C₁-C₆)alkylsulphinyl-(C₁-C₆)alkyl, (C₁-C₆)alkylsulphonyl, (C₁-C₆)haloalkylsulphonyl, (C₁-C₆)alkoxy-(C₁-C₆)alkylsulphonyl, (C₁-C₆)alkylsulphonyl-(C₁-C₆)alkyl, (C₁-C₆)alkylsulphonyloxy, (C₁-C₆)haloalkylsulphanyl, (C₃-C₆)cycloalkylsulphanyl, (C₃-C₆)halocycloalkylsulphanyl, (C₃-C₆)cycloalkylsulphonyl, (C₃-C₆)halocycloalkylsulphonyl, (C₃-C₆)cycloalkylsulphinyl, (C₃-C₆)halocycloalkylsulphinyl, (C₁-C₆)alkylcarbonyl, (C₁-C₆)haloalkylcarbonyl, (C₁-C₆)alkylcarbonyloxy, (C₁-C₆)alkoxycarbonyl, (C₁-C₆)haloalkoxycarbonyl, aminocarbonyl, (C₁-C₆)alkylaminocarbonyl, di-(C₁-C₆)alkylaminocarbonyl, (C₂-C₆)alkenylaminocarbonyl, di-(C₂-C₆)-alkenylaminocarbonyl, (C₃-C₈)cycloalkylaminocarbonyl, (C₁-C₆)alkylsulphonylamino, (C₁-C₆)alkylamino, di-(C₁-C₆)alkylamino, (C₁-C₆)haloalkylamino, bis-(C₁-C₆)haloalkylamino, aminosulphonyl, (C₁-C₆)alkylaminosulphonyl, di-(C₁-C₆)alkylaminosulphonyl, (C₁-C₆)alkylsulphoximino, aminothiocarbonyl, (C₁-C₆)alkylaminothiocarbonyl, di-(C₁-C₆)alkylaminothiocarbonyl, (C₃-C₈)cycloalkylamino,
R¹ is (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl, (C₃-C₈)cycloalkyl-(C₁-C₆)alkyl, (C₃-C₈)cycloalkyl-(C₃-C₈)cycloalkyl, (C₁-C₆)alkyl-(C₃-C₈)cycloalkyl, (C₁-C₆)haloalkyl-(C₃-C₈)cycloalkyl, halo(C₃-C₈)cycloalkyl, cyano(C₃-C₈)cycloalkyl, (C₂-C₆)alkynyl-(C₃-C₈)cycloalkyl, (C₁-C₆)alkoxy-(C₃-C₈)cycloalkyl, (C₁-C₆)alkoxycarbonyl-(C₃-C₈)cycloalkyl, (C₁-C₆)alkylamino, di-(C₁-C₆)alkylamino, (C₃-C₈)cycloalkylamino, (C₁-C₆)alkylcarbonylamino, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, (C₁-C₆)alkylsulphonylamino, aminosulphonyl-(C₁-C₆)alkyl, (C₁-C₆)alkylaminosulphonyl-(C₁-C₆)alkyl, di-(C₁-C₆)alkylaminosulphonyl-(C₁-C₆)alkyl, (C₁-C₄)alkoxy, (C₃-C₆)cycloalkoxy, (C₃-C₆)cycloalkylcarbonyl or (C₁-C₄)alkylcarbonyl,
   or in each case optionally singly or multiply, identically or differently aryl-, hetaryl-, oxohetaryl-, heterocyclyl- or oxoheterocyclyl-substituted (C₁-C₆)alkyl, (C₁-C₆)alkoxy, (C₂-C₆)alkenyl, (C₂-C₆)alkynyl, (C₃-C₈)cycloalkyl, where aryl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, aminosulphonyl, (C₁-C₆)alkyl, (C₃-C₆)cycloalkyl, (C₁-C₆)alkoxy, (C₁-C₆)haloalkyl, (C₁-C₆)haloalkoxy, (C₁-C₆)alkylthio, (C₁-C₆)alkylsulphinyl, (C₁-C₆)alkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl or (C₃-C₆)trialkylsilyl,
or in each case optionally singly or multiply, identically or differently substituted aryl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₈)cycloalkyl, (C₁-C₆)-alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl,
R² is hydrogen (C₁-C₆)alkyl, (C₃-C₈)cycloalkyl, (C₁-C₆)alkoxy, (C₁-C₆)alkylcarbonyl, (C₃-C₈)cycloalkylcarbonyl, (C₁-C₆)alkoxycarbonyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl-(C₃-C₈)cycloalkyl, (C₁-C₆)alkyl-(C₃-C₈)cycloalkyl, halo(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, aminocarbonyl-(C₁-C₆)alkyl, (C₁-C₆)alkylamino-(C₁-C₆)alkyl, di-(C₁-C₆)alkylamino-(C₁-C₆)alkyl or (C₃-C₈)cycloalkylamino-(C₁-C₆)alkyl,
R³ is hydrogen (C₁-C₆)alkyl, (C₃-C₈)cycloalkyl, (C₁-C₆)alkoxy, (C₁-C₆)alkylcarbonyl, (C₃-C₈)cycloalkylcarbonyl, (C₁-C₆)alkoxycarbonyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl-(C₃-C₈)cycloalkyl, (C₁-C₆)alkyl-(C₃-C₈)cycloalkyl, halo(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, aminocarbonyl-(C₁-C₆)alkyl, (C₁-C₆)alkylamino-(C₁-C₆)alkyl, di-(C₁-C₆)alkylamino-(C₁-C₆)alkyl or (C₃-C₈)cycloalkylamino-(C₁-C₆)alkyl,
R⁴ is (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl, (C₃-C₈)cycloalkyl-(C₁-C₆)alkyl, cyano(C₃-C₈)cycloalkyl-(C₁-C₆)alkyl, halo(C₃-C₈)cycloalkyl-(C₁-C₆)alkyl, (C₁-C₄)haloalkyl-(C₃-C₈)cycloalkyl-(C₁-C₆)alkyl, (C₃-C₈)cycloalkyl-(C₃-C₈)cycloalkyl, (C₁-C₆)alkyl-(C₃-C₈)cycloalkyl, (C₁-C₆)haloalkyl-(C₃-C₈)cycloalkyl, halo(C₃-C₈)cycloalkyl, cyano(C₃-C₈)cycloalkyl, (C₂-C₆)alkynyl-(C₃-C₈)cycloalkyl, (C₁-C₆)alkoxy-(C₃-C₈)cycloalkyl, (C₁-C₆)alkoxycarbonyl-(C₃-C₈)cycloalkyl, carbamoyl-(C₃-C₈)cycloalkyl, thiocarbamoyl-(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, (C₁-C₆)alkylsulphonylamino, aminosulphonyl-(C₁-C₆)alkyl, (C₁-C₆)alkylaminosulphonyl-(C₁-C₆)alkyl, di-(C₁-C₆)alkylaminosulphonyl-(C₁-C₆)alkyl, amino, (C₁-C₆)alkylamino, di-(C₁-C₆)alkylamino, (C₃-C₈)cycloalkylamino, N—(C₁-C₆)alkyl-(C₃-C₈)cycloalkylamino, benzylamino, cyanobenzylamino, nitrobenzylamino, halobenzylamino, N—(C₁-C₆)alkylbenzylamino, N—(C₁-C₆)alkylcyanobenzylamino, N—(C₁-C₆)alkylnitrobenzylamino, N—(C₁-C₆)alkylhalobenzylamino, (C₁-C₆)alkylcarbonylamino, (C₃-C₈)cycloalkylcarbonylamino, hydroxyl, (C₁-C₆)alkoxy, (C₃-C₈)cycloalkoxy, (C₃-C₈)cycloalkyl-(C₁-C₆)alkoxy, cyano(C₁-C₆)alkoxy, benzyloxy, cyanobenzyloxy, nitrobenzyloxy, halobenzyloxy, (C₁-C₆)alkylimino, (C₃-C₈)cycloalkylimino, benzylimino, cyanobenzylimino, nitrobenzylimino, halobenzylimino, (C₁-C₆)haloalkylbenzylimino, halo-[(C₁-C₆)haloalkyl]benzylimino, (C₁-C₆)alkylcarbonyl, (C₁-C₆)haloalkylcarbonyl, (C₃-C₈)cycloalkylcarbonyl or (C₃-C₈)cycloalkyl-(C₁-C₆)alkylcarbonyl,
   or in each case optionally singly or multiply, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₆)alkyl, (C₁-C₆)haloalkyl, (C₁-C₆)alkoxy, (C₂-C₆)alkenyl, (C₂-C₆)alkynyl, (C₃-C₈)cycloalkyl, oxy, amino, N—(C₁-C₆)alkylamino, N—(C₃-C₈)cycloalkylamino or carbonyl, where aryl, 1,3-benzodioxolyl, 2,3-dihydro-1,4-benzodioxinyl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, thiocarbamoyl, aminosulphonyl, (C₁-C₆)alkyl, (C₃-C₆)cycloalkyl, (C₁-C₆)alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)haloalkylsulphinyl, (C₁-C₆)haloalkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl or hetaryl, or
   in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₈)cycloalkyl, (C₁-C₆)-alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)haloalkylsulphinyl, (C₁-C₆)haloalkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl,
      or R³ and R⁴ together are (C₂-C₆)-alkyl or (C₂-C₆)alkenyl, forming a 3-7-membered, optionally halogen-, cyano-, hydroxyl-, amino-, carboxyl-, carbamoyl-, (C₁-C₆)alkyl-, (C₃-C₈)cycloalkyl-, (C₁-C₆)-alkoxy-, (C₁-C₆)haloalkyl-, (C₁-C₆)haloalkoxy-, (C₁-C₆)alkylthio- or (C₁-C₆)haloalkylthio-substituted ring which may optionally contain one to two double bonds,
R⁵, R⁶ are independently hydrogen, cyano, halogen, nitro, acetyl, hydroxyl, amino, (C₁-C₆)alkylamino, di-(C₁-C₆)alkylamino, (C₃-C₆)cycloalkyl, halo(C₃-C₆)cycloalkyl, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₁-C₆)alkoxy, (C₁-C₆)haloalkoxy, (C₁-C₆)alkylthio, (C₁-C₆)haloalkylthio, (C₁-C₆)alkylsulphinyl, (C₁-C₆)haloalkylsulphinyl, (C₁-C₆)alkylsulphonyl, (C₁-C₆)haloalkylsulphonyl,
V¹ is oxygen or sulphur,
V² is oxygen, sulphur or —NH.

It has additionally been found that the compounds of the formula (I) have very good efficacy as pesticides, preferably as insecticides and/or acaricides, and additionally generally have very good plant compatibility, in particular with respect to crop plants.

The compounds according to the invention are defined in general terms by the formula (I). Preferred substituents or ranges for the radicals listed in the formulae mentioned above and below are illustrated hereinafter:

### Preferred Range:

Preferred compounds (Configuration 2-1) are those of the formula (I) in which
Q¹ is in each case optionally singly or multiply, identically or differently substituted phenyl, 1,3-benzodioxolyl, 2,3-dihydro-1,4-benzodioxinyl, hetaryl or oxohetaryl,
   where the substituents are selected from cyano, halogen, nitro, amino, SF₅, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₂-C₄)alkenyl, (C₂-C₄)haloalkenyl, (C₂-C₄)cyanoalkenyl, (C₂-C₄)alkynyl, (C₂-C₄)haloalkynyl, (C₂-C₄)cyanoalkynyl, (C₁-C₄)alkoxy, (C₁-C₄)haloalkoxy, (C₁-C₄)cyanoalkoxy, (C₁-C₄)alkoxy-(C₁-C₄)alkoxy, (C₃-C₆)cycloalkyl, (C₃-C₆)halocycloalkyl, (C₁-C₄)alkyl-(C₃-C₆)cycloalkyl, (C₁-C₄)haloalkyl-(C₃-C₆)cycloalkyl, (C₁-C₄)haloalkenoxy, (C₁-C₄)alkylhydroxyimino, (C₁-C₄)alkoxyimino, (C₁-C₄)alkyl-(C₁-C₄)alkoxyimino, (C₁-C₄)haloalkyl-(C₁-C₄)alkoxyimino, (C₁-C₄)alkylsulphanyl, (C₁-C₄)alkylsulphanyl-(C₁-C₄)alkyl, (C₁-C₄)alkylsulphinyl, (C₁-C₄)haloalkylsulphinyl, (C₁-C₄)alkylsulphinyl-(C₁-C₄)alkyl, (C₁-C₄)alkylsulphonyl, (C₁-C₄)haloalkylsulphonyl, (C₁-C₄)alkylsulphonyl-(C₁-C₄)alkyl, (C₁-C₄)alkylsulphonyloxy, (C₁-C₄)haloalkylsulphanyl, (C₃-C₆)cycloalkylsulphanyl, (C₃-C₆)halocycloalkylsulphanyl, (C₃-C₆)cycloalkylsulphonyl, (C₃-C₆)halocycloalkylsulphonyl, (C₃-C₆)cycloalkylsulphinyl, (C₃-C₆)halocycloalkylsulphinyl, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, (C₁-C₆)haloalkylamino, bis-(C₁-C₆)haloalkylamino, aminosulphonyl, (C₁-C₄)alkylaminosulphonyl, di-(C₁-C₄)alkylaminosulphonyl,
R¹ is (C₁-C₆)alkyl, (C₁-C₆)haloalkyl, (C₁-C₆)cyanoalkyl, (C₁-C₆)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₆)alkyl, (C₂-C₄)haloalkenyloxy-(C₁-C₆)alkyl, (C₂-C₄)haloalkenyl, (C₂-C₄)cyanoalkenyl, (C₂-C₄)alkynyl, (C₂-C₄)alkynyloxy-(C₁-C₆)alkyl, (C₂-C₄)haloalkynyloxy-(C₁-C₄)alkyl, (C₂-C₄)haloalkynyl, (C₂-C₄)cyanoalkynyl, (C₃-C₆)cycloalkyl, (C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, (C₁-C₆)alkylamino, di-(C₁-C₆)alkylamino, (C₃-C₆)cycloalkylamino, (C₁-C₄)alkylthio-(C₁-C₆)alkyl, (C₁-C₄)haloalkylthio-(C₁-C₆)alkyl, (C₁-C₄)alkylsulphinyl-(C₁-C₆)alkyl, (C₁-C₄)haloalkylsulphinyl-(C₁-C₆)alkyl, (C₁-C₄)alkylsulphonyl-(C₁-C₆)alkyl, (C₁-C₄)haloalkylsulphonyl-(C₁-C₆)alkyl, (C₁-C₄)alkylcarbonyl-(C₁-C₆)alkyl, (C₁-C₄)alkoxycarbonyl-(C₁-C₆)alkyl, (C₁-C₄)alkoxy, (C₃-C₆)cycloalkoxy, (C₃-C₆)cycloalkylcarbonyl or (C₁-C₄)alkylcarbonyl,
   or is in each case optionally singly or multiply, identically or differently aryl-, hetaryl-, oxohetaryl-, heterocyclyl- or oxoheterocyclyl-substituted (C₁-C₄)alkyl, (C₁-C₄)alkoxy, (C₂-C₄)alkenyl, (C₂-C₄)alkynyl, (C₃-C₆)cycloalkyl, where aryl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)alkylsulphinyl, (C₁-C₄)alkylsulphonyl, (C₁-C₆)alkoxycarbonyl, (C₁-C₆)alkylcarbonyl or (C₃-C₆)trialkylsilyl,
   or is in each case optionally singly or multiply, identically or differently substituted aryl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₆)alkoxycarbonyl, (C₁-C₆)alkylcarbonyl, (C₃-C₆)trialkylsilyl,
R² is hydrogen, (C₁-C₆)alkyl, (C₃-C₆)cycloalkyl, (C₁-C₄)alkoxy, (C₁-C₆)alkylcarbonyl, (C₃-C₆)cycloalkylcarbonyl, (C₁-C₆)alkoxycarbonyl, (C₁-C₄)haloalkyl or (C₁-C₄)cyanoalkyl,
R³ is hydrogen, (C₁-C₆)alkyl, (C₃-C₆)cycloalkyl, (C₁-C₄)alkoxy, (C₁-C₆)alkylcarbonyl, (C₃-C₆)cycloalkylcarbonyl, (C₁-C₆)alkoxycarbonyl, (C₁-C₄)haloalkyl or (C₁-C₄)cyanoalkyl,
R⁴ is (C₁-C₆)alkyl, (C₁-C₆)haloalkyl, (C₁-C₆)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₆)alkenyl, (C₂-C₄)alkenyloxy-(C₁-C₆)alkyl, (C₂-C₄)haloalkenyloxy-(C₁-C₄)alkyl, (C₂-C₄)haloalkenyl, (C₂-C₆)cyanoalkenyl, (C₂-C₆)alkynyl, (C₂-C₄)alkynyloxy-(C₁-C₄)alkyl, (C₂-C₄)haloalkynyloxy-(C₁-C₄)alkyl, (C₂-C₆)haloalkynyl, (C₂-C₆)cyanoalkynyl, (C₃-C₆)cycloalkyl, (C₃-C₆)cycloalkyl-(C₁-C₆)alkyl, cyano(C₃-C₆)cycloalkyl-(C₁-C₆)alkyl, halo(C₃-C₆)cycloalkyl-(C₁-C₆)alkyl, (C₁-C₄)haloalkyl-(C₃-C₆)cycloalkyl-(C₁-C₆)alkyl, (C₃-C₆)cycloalkyl-(C₃-C₆)cycloalkyl, (C₁-C₆)alkyl-(C₃-C₆)cycloalkyl, (C₁-C₆)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, carbamoyl-(C₃-C₆)cycloalkyl, thiocarbamoyl-(C₃-C₆)cycloalkyl, (C₁-C₄)alkylthio-(C₁-C₄)alkyl, (C₁-C₄)haloalkylthio-(C₁-C₄)alkyl, (C₁-C₄)alkylsulphinyl-(C₁-C₄)alkyl, (C₁-C₄)haloalkylsulphinyl-(C₁-C₄)alkyl, (C₁-C₄)alkylsulphonyl-(C₁-C₄)alkyl, (C₁-C₄)haloalkylsulphonyl-(C₁-C₄)alkyl, (C₁-C₆)alkylcarbonyl-(C₁-C₆)alkyl, (C₁-C₆)haloalkylcarbonyl-(C₁-C₆)alkyl, (C₁-C₄)alkoxycarbonyl-(C₁-C₄)alkyl, (C₁-C₄)haloalkoxycarbonyl-(C₁-C₄)alkyl, (C₁-C₄)alkylsulphonylamino, aminosulphonyl-(C₁-C₄)alkyl, (C₁-C₄)alkylaminosulphonyl-(C₁-C₄)alkyl, or di-(C₁-C₄)alkylaminosulphonyl-(C₁-C₆)alkyl, amino, (C₁-C₄)alkylamino, di-(C₁-C₄)alkylamino, (C₃-C₆)cycloalkylamino, N—(C₁-C₄)alkyl-(C₃-C₆)cycloalkylamino, benzylamino, cyanobenzylamino, nitrobenzylamino, halobenzylamino, N—(C₁-C₄)alkylbenzylamino, N—(C₁-C₄)alkylcyanobenzylamino, N—(C₁-C₄)alkylnitrobenzylamino, N—(C₁-C₄)alkylhalobenzylamino, (C₁-C₄)alkylcarbonylamino, (C₃-C₆)cycloalkylcarbonylamino, hydroxyl, (C₁-C₄)alkoxy, (C₃-C₆)cycloalkoxy, (C₃-C₆)cycloalkyl-(C₁-C₄)alkoxy, benzyloxy, cyanobenzyloxy, nitrobenzyloxy, halobenzyloxy, (C₁-C₄)alkylimino, (C₃-C₆)cycloalkylimino, benzylimino, cyanobenzylimino, nitrobenzylimino or halobenzylimino,
   or in each case optionally singly or multiply, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₄)alkyl, (C₁-C₄)haloalkyl, (C₁-C₄)alkoxy, (C₂-C₄)alkenyl, (C₂-C₄)alkynyl, (C₃-C₆)cycloalkyl, oxy, amino, N—(C₁-C₄)alkylamino or N—(C₃-C₆)cycloalkylamino, where aryl, 1,3-benzodioxolyl, 2,3-dihydro-1,4-benzodioxinyl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, thiocarbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkylsulphimino, (C₁-C₄)alkylsulphimino-(C₁-C₄)alkyl, (C₁-C₄)alkylsulphoximino, (C₁-C₄)alkylsulphoximino-(C₁-C₄)alkyl, (C₁-C₄)alkoxycarbonyl, (C₁-C₄)alkylcarbonyl, (C₃-C₆)trialkylsilyl or hetaryl, or
   in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkylsulphimino, (C₁-C₄)alkylsulphimino-(C₁-C₄)alkyl, (C₁-C₄)alkylsulphoximino, (C₁-C₄)alkylsulphoximino-(C₁-C₄)alkyl, (C₁-C₄)alkoxycarbonyl, (C₁-C₄)alkylcarbonyl, (C₃-C₆)trialkylsilyl,
      or R³ and R⁴ together are (C₂-C₆)-alkyl or (C₂-C₆)alkenyl, forming a 3-7-membered, optionally halogen-, cyano-, hydroxyl-, amino-, (C₁-C₄)alkyl-, (C₃-C₆)cycloalkyl-, (C₁-C₄)-alkoxy-, (C₁-C₄)haloalkyl-, (C₁-C₄)haloalkoxy-, (C₁-C₄)alkylthio- or (C₁-C₄)haloalkylthio-substituted ring which may optionally contain one to two double bonds,
R⁵, R⁶ are independently hydrogen, cyano, halogen, nitro, (C₃-C₆)cycloalkyl, (C₁-C₄)alkyl, (C₁-C₄)haloalkyl or (C₁-C₄)haloalkoxy,
V¹, V² are independently oxygen or sulphur.

Alternative and likewise preferred compounds (Configuration 2-2) are those of the formula (I) in which
Q¹ is in each case optionally singly or multiply, identically or differently substituted phenyl, 1,3-benzodioxolyl, 2,3-dihydro-1,4-benzodioxinyl, hetaryl or oxohetaryl,
   where the substituents are selected from cyano, halogen, nitro, amino, SF₅, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₂-C₄)alkenyl, (C₂-C₄)haloalkenyl, (C₂-C₄)cyanoalkenyl, (C₂-C₄)alkynyl, (C₂-C₄)haloalkynyl, (C₂-C₄)cyanoalkynyl, (C₁-C₄)alkoxy, (C₁-C₄)haloalkoxy, (C₁-C₄)cyanoalkoxy, (C₁-C₄)alkoxy-(C₁-C₄)alkoxy, (C₃-C₆)cycloalkyl, (C₃-C₆)halocycloalkyl, (C₁-C₄)alkyl-(C₃-C₆)cycloalkyl, (C₁-C₄)haloalkyl-(C₃-C₆)cycloalkyl, (C₁-C₄)haloalkenoxy, (C₁-C₄)alkylhydroxyimino, (C₁-C₄)alkoxyimino, (C₁-C₄)alkyl-(C₁-C₄)alkoxyimino, (C₁-C₄)haloalkyl-(C₁-C₄)alkoxyimino, (C₁-C₄)alkylsulphanyl, (C₁-C₄)alkylsulphanyl-(C₁-C₄)alkyl, (C₁-C₄)alkylsulphinyl, (C₁-C₄)haloalkylsulphinyl, (C₁-C₄)alkylsulphinyl-(C₁-C₄)alkyl, (C₁-C₄)alkylsulphonyl, (C₁-C₄)haloalkylsulphonyl, (C₁-C₄)alkylsulphonyl-(C₁-C₄)alkyl, (C₁-C₄)alkylsulphonyloxy, (C₁-C₄)haloalkylsulphanyl, (C₃-C₆)cycloalkylsulphanyl, (C₃-C₆)halocycloalkylsulphanyl, (C₃-C₆)cycloalkylsulphonyl, (C₃-C₆)halocycloalkylsulphonyl, (C₃-C₆)cycloalkylsulphinyl, (C₃-C₆)halocycloalkylsulphinyl, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, (C₁-C₆)haloalkylamino, bis-(C₁-C₆)haloalkylamino, aminosulphonyl, (C₁-C₄)alkylaminosulphonyl, di-(C₁-C₄)alkylaminosulphonyl,
R¹ is (C₁-C₆)alkyl, (C₁-C₆)haloalkyl, (C₁-C₆)cyanoalkyl, (C₁-C₆)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₆)alkyl, (C₂-C₄)haloalkenyloxy-(C₁-C₆)alkyl, (C₂-C₄)haloalkenyl, (C₂-C₄)cyanoalkenyl, (C₂-C₄)alkynyl, (C₂-C₄)alkynyloxy-(C₁-C₆)alkyl, (C₂-C₄)haloalkynyloxy-(C₁-C₄)alkyl, (C₂-C₄)haloalkynyl, (C₂-C₄)cyanoalkynyl, (C₃-C₆)cycloalkyl, (C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, (C₁-C₆)alkylamino, di-(C₁-C₆)alkylamino, (C₃-C₆)cycloalkylamino, (C₁-C₄)alkylthio-(C₁-C₆)alkyl, (C₁-C₄)haloalkylthio-(C₁-C₆)alkyl, (C₁-C₄)alkylsulphinyl-(C₁-C₆)alkyl, (C₁-C₄)haloalkylsulphinyl-(C₁-C₆)alkyl, (C₁-C₄)alkylsulphonyl-(C₁-C₆)alkyl, (C₁-C₄)haloalkylsulphonyl-(C₁-C₆)alkyl, (C₁-C₄)alkylcarbonyl-(C₁-C₆)alkyl, (C₁-C₄)alkoxycarbonyl-(C₁-C₆)alkyl, (C₁-C₄)alkoxy, (C₃-C₆)cycloalkoxy, (C₃-C₆)cycloalkylcarbonyl or (C₁-C₄)alkylcarbonyl,
   or is in each case optionally singly or multiply, identically or differently aryl-, hetaryl-, oxohetaryl-, heterocyclyl- or oxoheterocyclyl-substituted (C₁-C₄)alkyl, (C₁-C₄)alkoxy, (C₂-C₄)alkenyl, (C₂-C₄)alkynyl, (C₃-C₆)cycloalkyl, where aryl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)alkylsulphinyl, (C₁-C₄)alkylsulphonyl, (C₁-C₆)alkoxycarbonyl, (C₁-C₆)alkylcarbonyl or (C₃-C₆)trialkylsilyl,
   or is in each case optionally singly or multiply, identically or differently substituted aryl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₆)alkoxycarbonyl, (C₁-C₆)alkylcarbonyl, (C₃-C₆)trialkylsilyl,
R² is hydrogen, (C₁-C₆)alkyl, (C₃-C₆)cycloalkyl, (C₁-C₄)alkoxy, (C₁-C₆)alkylcarbonyl, (C₃-C₆)cycloalkylcarbonyl, (C₁-C₆)alkoxycarbonyl, (C₁-C₄)haloalkyl or (C₁-C₄)cyanoalkyl,
R³ is hydrogen, (C₁-C₆)alkyl, (C₃-C₆)cycloalkyl, (C₁-C₄)alkoxy, (C₁-C₆)alkylcarbonyl, (C₃-C₆)cycloalkylcarbonyl, (C₁-C₆)alkoxycarbonyl, (C₁-C₄)haloalkyl or (C₁-C₄)cyanoalkyl,
R⁴ is (C₁-C₆)alkyl, (C₁-C₆)haloalkyl, (C₁-C₆)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₆)alkenyl, (C₂-C₄)alkenyloxy-(C₁-C₆)alkyl, (C₂-C₄)haloalkenyloxy-(C₁-C₄)alkyl, (C₂-C₄)haloalkenyl, (C₂-C₆)cyanoalkenyl, (C₂-C₆)alkynyl, (C₂-C₄)alkynyloxy-(C₁-C₄)alkyl, (C₂-C₄)haloalkynyloxy-(C₁-C₄)alkyl, (C₂-C₆)haloalkynyl, (C₂-C₆)cyanoalkynyl, (C₃-C₆)cycloalkyl, (C₃-C₆)cycloalkyl-(C₁-C₆)alkyl, cyano(C₃-C₆)cycloalkyl-(C₁-C₆)alkyl, halo(C₃-C₆)cycloalkyl-(C₁-C₆)alkyl, (C₁-C₄)haloalkyl-(C₃-C₆)cycloalkyl-(C₁-C₆)alkyl, (C₃-C₆)cycloalkyl-(C₃-C₆)cycloalkyl, (C₁-C₆)alkyl-(C₃-C₆)cycloalkyl, (C₁-C₆)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, carbamoyl-(C₃-C₆)cycloalkyl, thiocarbamoyl-(C₃-C₆)cycloalkyl, (C₁-C₄)alkylthio-(C₁-C₄)alkyl, (C₁-C₄)haloalkylthio-(C₁-C₄)alkyl, (C₁-C₄)alkylsulphinyl-(C₁-C₄)alkyl, (C₁-C₄)haloalkylsulphinyl-(C₁-C₄)alkyl, (C₁-C₄)alkylsulphonyl-(C₁-C₄)alkyl, (C₁-C₄)haloalkylsulphonyl-(C₁-C₄)alkyl, (C₁-C₆)alkylcarbonyl-(C₁-C₆)alkyl, (C₁-C₆)haloalkylcarbonyl-(C₁-C₆)alkyl, (C₁-C₄)alkoxycarbonyl-(C₁-C₄)alkyl, (C₁-C₄)haloalkoxycarbonyl-(C₁-C₄)alkyl, (C₁-C₄)alkylsulphonylamino, aminosulphonyl-(C₁-C₄)alkyl, (C₁-C₄)alkylaminosulphonyl-(C₁-C₄)alkyl, or di-(C₁-C₄)alkylaminosulphonyl-(C₁-C₆)alkyl, amino, (C₁-C₄)alkylamino, di-(C₁-C₄)alkylamino, (C₃-C₆)cycloalkylamino, N—(C₁-C₄)alkyl-(C₃-C₆)cycloalkylamino, benzylamino, cyanobenzylamino, nitrobenzylamino, halobenzylamino, N—(C₁-C₄)alkylbenzylamino, N—(C₁-C₄)alkylcyanobenzylamino, N—(C₁-C₄)alkylnitrobenzylamino, N—(C₁-C₄)alkylhalobenzylamino, (C₁-C₄)alkylcarbonylamino, (C₃-C₆)cycloalkylcarbonylamino, hydroxyl, (C₁-C₄)alkoxy, (C₃-C₆)cycloalkoxy, (C₃-C₆)cycloalkyl-(C₁-C₄)alkoxy, cyano(C₁-C₄)alkoxy, benzyloxy, cyanobenzyloxy, nitrobenzyloxy, halobenzyloxy, (C₁-C₄)alkylimino, (C₃-C₆)cycloalkylimino, benzylimino, cyanobenzylimino, nitrobenzylimino, halobenzylimino, (C₁-C₄)haloalkylbenzylimino, halo-[(C₁-C₄)haloalkyl]benzylimino, (C₁-C₄)alkylcarbonyl, (C₁-C₄)haloalkylcarbonyl, (C₃-C₆)cycloalkylcarbonyl or (C₃-C₆)cycloalkyl-(C₁-C₄)alkylcarbonyl,
   or in each case optionally singly or multiply, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₄)alkyl, (C₁-C₄)haloalkyl, (C₁-C₄)alkoxy, (C₂-C₄)alkenyl, (C₂-C₄)alkynyl, (C₃-C₆)cycloalkyl, oxy, amino, N—(C₁-C₄)alkylamino, N—(C₃-C₆)cycloalkylamino or carbonyl, where aryl, 1,3-benzodioxolyl, 2,3-dihydro-1,4-benzodioxinyl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, thiocarbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkylsulphimino, (C₁-C₄)alkylsulphimino-(C₁-C₄)alkyl, (C₁-C₄)alkylsulphoximino, (C₁-C₄)alkylsulphoximino-(C₁-C₄)alkyl, (C₁-C₄)alkoxycarbonyl, (C₁-C₄)alkylcarbonyl, (C₃-C₆)trialkylsilyl or hetaryl, or
   or in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkylsulphimino, (C₁-C₄)alkylsulphimino-(C₁-C₄)alkyl, (C₁-C₄)alkylsulphoximino, (C₁-C₄)alkylsulphoximino-(C₁-C₄)alkyl, (C₁-C₄)alkoxycarbonyl, (C₁-C₄)alkylcarbonyl, (C₃-C₆)trialkylsilyl,
      or R³ and R⁴ together are (C₂-C₆)-alkyl or (C₂-C₆)alkenyl, forming a 3-7-membered, optionally halogen-, cyano-, hydroxyl-, amino-, (C₁-C₄)alkyl-, (C₃-C₆)cycloalkyl-, (C₁-C₄)alkoxy-, (C₁-C₄)haloalkyl-, (C₁-C₄)haloalkoxy-, (C₁-C₄)alkylthio- or (C₁-C₄)haloalkylthio-substituted ring which may optionally contain one to two double bonds,
R⁵, R⁶ are independently hydrogen, cyano, halogen, nitro, (C₃-C₆)cycloalkyl, (C₁-C₄)alkyl, (C₁-C₄)haloalkyl or (C₁-C₄)haloalkoxy,
V¹ is oxygen or sulphur,
V² is oxygen, sulphur or —NH.

### Particularly Preferred Range:

Particularly preferred compounds (Configuration 3-1) are those of the formula (I) in which
Q¹ is in each case optionally singly or multiply, identically or differently substituted phenyl, 1,3-benzodioxolyl, 2,3-dihydro-1,4-benzodioxinyl, pyrimidyl, pyridazinyl, pyridyl, thienyl, furanyl, oxazolyl, thiazolyl, imidazolyl or pyrazolyl,
   where the substituents are selected from cyano, halogen, nitro, SF₅, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₁-C₄)alkoxy, (C₁-C₄)haloalkoxy, (C₃-C₆)cycloalkyl, (C₃-C₆)halocycloalkyl, (C₁-C₄)alkyl-(C₃-C₆)cycloalkyl, (C₁-C₄)haloalkyl-(C₃-C₆)cycloalkyl, (C₁-C₄)haloalkenoxy, (C₁-C₄)alkylsulphanyl, (C₃-C₆)halocycloalkylsulphanyl, (C₁-C₄)alkylsulphinyl, (C₁-C₄)haloalkylsulphinyl, (C₃-C₆)cycloalkylsulphinyl, (C₁-C₄)alkylsulphonyl, (C₁-C₄)haloalkylsulphonyl, (C₃-C₆)cycloalkylsulphonyl, (C₁-C₄)haloalkylsulphanyl, (C₃-C₆)cycloalkylsulphanyl, (C₁-C₄)alkylcarbonyl, (C₁-C₄)haloalkylcarbonyl, bis-(C₁-C₄)alkylamino, di-(C₁-C₄)haloalkylamino,
R¹ is (C₁-C₄)alkyl, (C₁-C₄)haloalkyl, (C₁-C₄)hydroxyalkyl, (C₁-C₄)alkoxy-(C₁-C₄)alkyl, (C₂-C₄)alkenyl, (C₂-C₄)haloalkenyl, (C₂-C₄)alkynyl, (C₂-C₄)alkynyloxy-(C₁-C₄)alkyl, (C₃-C₆)cycloalkyl, (C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, (C₁-C₄)alkylthio-(C₁-C₆)alkyl, (C₁-C₄)alkylcarbonyl-(C₁-C₆)alkyl, (C₁-C₄)alkoxycarbonyl-(C₁-C₆)alkyl or (C₁-C₄)alkoxy,
   or in each case singly or multiply, identically or differently aryl-, hetaryl- or heterocyclyl-substituted (C₁-C₄)alkyl, (C₃-C₆)cycloalkyl, where aryl, hetaryl or heterocyclyl may each optionally be mono- or polysubstituted identically or differently by halogen, cyano, nitro, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio or (C₁-C₆)alkoxycarbonyl,
   or is in each case optionally singly or multiply, identically or differently substituted aryl, hetaryl, heterocyclyl or oxoheterocyclyl, where the substituents are selected from halogen, cyano, nitro, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₆)alkoxycarbonyl,
R² is hydrogen, (C₁-C₄)alkyl, (C₃-C₆)cycloalkyl, (C₁-C₄)alkoxy, (C₁-C₄)alkylcarbonyl, (C₃-C₆)cycloalkylcarbonyl or (C₁-C₄)alkoxycarbonyl,
R³ is hydrogen, (C₁-C₄)alkyl, (C₃-C₆)cycloalkyl, (C₁-C₄)alkoxy, (C₁-C₄)alkylcarbonyl, (C₃-C₆)cycloalkylcarbonyl or (C₁-C₄)alkoxycarbonyl,
R⁴ is (C₁-C₆)alkyl, (C₁-C₆)haloalkyl, (C₁-C₆)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₆)haloalkynyl, (C₂-C₆)cyanoalkynyl, (C₃-C₆)cycloalkyl, (C₃-C₆)cycloalkyl-(C₁-C₄)alkyl, cyano(C₃-C₆)cycloalkyl-(C₁-C₄)alkyl, halo(C₃-C₆)cycloalkyl-(C₁-C₄)alkyl, (C₁-C₄)haloalkyl-(C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, carbamoyl-(C₃-C₆)cycloalkyl, thiocarbamoyl-(C₃-C₆)cycloalkyl, (C₁-C₄)alkylcarbonyl-(C₁-C₄)alkyl, (C₁-C₄)alkoxycarbonyl-(C₁-C₄)alkyl, amino, (C₁-C₄)alkylamino, di-(C₁-C₄)alkyl-amino, (C₃-C₆)cycloalkylamino, N—(C₁-C₄)alkyl-(C₃-C₆)cycloalkylamino, benzylamino, cyanobenzylamino, nitrobenzylamino, N—(C₁-C₄)alkylbenzylamino, N—(C₁-C₄)alkylcyanobenzylamino, N—(C₁-C₄)alkylnitrobenzylamino, (C₁-C₄)alkylcarbonylamino, (C₃-C₆)cycloalkylcarbonylamino, hydroxyl, (C₁-C₄)alkoxy, (C₃-C₆)cycloalkoxy, (C₃-C₆)cycloalkyl-(C₁-C₄)alkoxy, benzyloxy, cyanobenzyloxy, nitrobenzyloxy, (C₁-C₄)alkylimino, (C₃-C₆)cycloalkylimino, benzylimino, cyanobenzylimino or nitrobenzylimino,
   or is in each case optionally singly or doubly, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₄)alkyl, (C₁-C₄)haloalkyl, (C₂-C₄)alkenyl, (C₃-C₆)cycloalkyl, oxy, amino, N—(C₁-C₄)alkylamino or N-cyclopropylamino, where aryl, 1,3-benzodioxolyl, 2,3-dihydro-1,4-benzodioxinyl, hetaryl, oxohetaryl, heterocyclyl or oxoheterocyclyl may each optionally be singly or multiply, identically or differently substituted by halogen, cyano, nitro, hydroxyl, amino, carboxyl, carbamoyl, thiocarbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkoxycarbonyl, (C₁-C₄)alkylcarbonyl, hetaryl or alkylhetaryl,
   or is in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkoxycarbonyl, (C₁-C₄)alkylcarbonyl, (C₃-C₆)trialkylsilyl,
      or R³ and R⁴ together are (C₂-C₅)-alkyl or (C₂-C₅)alkenyl, forming a 3-6-membered, optionally halogen-, cyano-, (C₁-C₄)alkyl-, (C₃-C₆)cycloalkyl-, (C₁-C₄)-alkoxy-, (C₁-C₄)haloalkyl-, (C₁-C₄)haloalkoxy- or (C₁-C₄)haloalkylthio-substituted ring which may optionally contain one to two double bonds,
R⁵, R⁶ are independently hydrogen, cyano, halogen, methyl, ethyl, difluoromethyl, trifluoromethyl or trifluoromethoxy,
V¹, V² are independently oxygen or sulphur.

Alternative and likewise particularly preferred compounds (Configuration 3-2) are those of the formula (I) in which
Q¹ is in each case optionally singly or multiply, identically or differently substituted phenyl, 1,3-benzodioxolyl, 2,3-dihydro-1,4-benzodioxinyl, pyrimidyl, pyridazinyl, pyridyl, thienyl, furanyl, oxazolyl, thiazolyl, imidazolyl or pyrazolyl,
   where the substituents are selected from cyano, halogen, nitro, SF₅, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₁-C₄)alkoxy, (C₁-C₄)haloalkoxy, (C₃-C₆)cycloalkyl, (C₃-C₆)halocycloalkyl, (C₁-C₄)alkyl-(C₃-C₆)cycloalkyl, (C₁-C₄)haloalkyl-(C₃-C₆)cycloalkyl, (C₁-C₄)haloalkenoxy, (C₁-C₄)alkylsulphanyl, (C₃-C₆)halocycloalkylsulphanyl, (C₁-C₄)alkylsulphinyl, (C₁-C₄)haloalkylsulphinyl, (C₃-C₆)cycloalkylsulphinyl, (C₁-C₄)alkylsulphonyl, (C₁-C₄)haloalkylsulphonyl, (C₃-C₆)cycloalkylsulphonyl, (C₁-C₄)haloalkylsulphanyl, (C₃-C₆)cycloalkylsulphanyl, (C₁-C₄)alkylcarbonyl, (C₁-C₄)haloalkylcarbonyl, bis-(C₁-C₄)alkylamino, di-(C₁-C₄)haloalkylamino,
R¹ is (C₁-C₄)alkyl, (C₁-C₄)haloalkyl, (C₁-C₄)hydroxyalkyl, (C₁-C₄)alkoxy-(C₁-C₄)alkyl, (C₂-C₄)alkenyl, (C₂-C₄)haloalkenyl, (C₂-C₄)alkynyl, (C₂-C₄)alkynyloxy-(C₁-C₄)alkyl, (C₃-C₆)cycloalkyl, (C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, (C₁-C₄)alkylthio-(C₁-C₆)alkyl, (C₁-C₄)alkylcarbonyl-(C₁-C₆)alkyl, (C₁-C₄)alkoxycarbonyl-(C₁-C₆)alkyl or (C₁-C₄)alkoxy,
   or in each case singly or multiply, identically or differently aryl-, hetaryl- or heterocyclyl-substituted (C₁-C₄)alkyl, (C₃-C₆)cycloalkyl, where aryl, hetaryl or heterocyclyl may each optionally be mono- or polysubstituted identically or differently by halogen, cyano, nitro, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio or (C₁-C₆)alkoxycarbonyl,
   or is in each case optionally singly or multiply, identically or differently substituted aryl, hetaryl, heterocyclyl or oxoheterocyclyl, where the substituents are selected from halogen, cyano, nitro, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₆)alkoxycarbonyl,
R² is hydrogen, (C₁-C₄)alkyl, (C₃-C₆)cycloalkyl, (C₁-C₄)alkoxy, (C₁-C₄)alkylcarbonyl, (C₃-C₆)cycloalkylcarbonyl or (C₁-C₄)alkoxycarbonyl,
R³ is hydrogen, (C₁-C₄)alkyl, (C₃-C₆)cycloalkyl, (C₁-C₄)alkoxy, (C₁-C₄)alkylcarbonyl, (C₃-C₆)cycloalkylcarbonyl or (C₁-C₄)alkoxycarbonyl,
R⁴ is (C₁-C₆)alkyl, (C₁-C₆)haloalkyl, (C₁-C₆)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₆)haloalkynyl, (C₂-C₆)cyanoalkynyl, (C₃-C₆)cycloalkyl, (C₃-C₆)cycloalkyl-(C₁-C₄)alkyl, cyano(C₃-C₆)cycloalkyl-(C₁-C₄)alkyl, halo(C₃-C₆)cycloalkyl-(C₁-C₄)alkyl, (C₁-C₄)haloalkyl-(C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, carbamoyl-(C₃-C₆)cycloalkyl, thiocarbamoyl-(C₃-C₆)cycloalkyl, (C₁-C₄)alkylcarbonyl-(C₁-C₄)alkyl, (C₁-C₄)alkoxycarbonyl-(C₁-C₄)alkyl, amino, (C₁-C₄)alkylamino, di-(C₁-C₄)alkyl-amino, (C₃-C₆)cycloalkylamino, N—(C₁-C₄)alkyl-(C₃-C₆)cycloalkylamino, benzylamino, cyanobenzylamino, nitrobenzylamino, N—(C₁-C₄)alkylbenzylamino, N—(C₁-C₄)alkylcyanobenzylamino, N—(C₁-C₄)alkylnitrobenzylamino, (C₁-C₄)alkylcarbonylamino, (C₃-C₆)cycloalkylcarbonylamino, hydroxyl, (C₁-C₄)alkoxy, (C₃-C₆)cycloalkoxy, (C₃-C₆)cycloalkyl-(C₁-C₄)alkoxy, cyano(C₁-C₄)alkoxy, benzyloxy, cyanobenzyloxy, nitrobenzyloxy, (C₁-C₄)alkylimino, (C₃-C₆)cycloalkylimino, benzylimino, cyanobenzylimino, halobenzylimino, (C₁-C₄)haloalkylbenzylimino, halo-[(C₁-C₄)haloalkyl]benzylimino, nitrobenzylimino, (C₁-C₄)alkylsulphonyl-(C₁-C₄)alkyl, (C₁-C₄)alkylcarbonyl, (C₁-C₄)haloalkylcarbonyl, (C₃-C₆)cycloalkylcarbonyl or (C₃-C₆)cycloalkyl-(C₁-C₄)alkylcarbonyl,
   or is in each case optionally singly or doubly, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₄)alkyl, (C₁-C₄)haloalkyl, (C₂-C₄)alkenyl, (C₃-C₆)cycloalkyl, oxy, amino, N—(C₁-C₄)alkylamino, N-cyclopropylamino or carbonyl, where aryl, 1,3-benzodioxolyl, 2,3-dihydro-1,4-benzodioxinyl, hetaryl, oxohetaryl, heterocyclyl or oxoheterocyclyl may each optionally be singly or multiply, identically or differently substituted by halogen, cyano, nitro, hydroxyl, amino, carboxyl, carbamoyl, thiocarbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkoxycarbonyl, (C₁-C₄)alkylcarbonyl, hetaryl, alkylhetaryl, heterocyclyl or oxoheterocyclyl,
   or is in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkoxycarbonyl, (C₁-C₄)alkylcarbonyl, (C₃-C₆)trialkylsilyl,
      or R³ and R⁴ together are (C₂-C₅)-alkyl or (C₂-C₅)alkenyl, forming a 3-6-membered, optionally halogen-, cyano-, (C₁-C₄)alkyl-, (C₃-C₆)cycloalkyl-, (C₁-C₄)-alkoxy-, (C₁-C₄)haloalkyl-, (C₁-C₄)haloalkoxy- or (C₁-C₄)haloalkylthio-substituted ring which may optionally contain one to two double bonds,
R⁵, R⁶ are independently hydrogen, cyano, halogen, methyl, ethyl, difluoromethyl, trifluoromethyl or trifluoromethoxy,
V¹ is oxygen or sulphur,
V² is oxygen, sulphur or —NH.

### Very Particularly Preferred Range:

Very particularly preferred compounds (Configuration 4-1) are those of the formula (I) in which
Q¹ is in each case optionally singly or multiply, identically or differently substituted phenyl, 1,3-benzodioxolyl, 2,3-dihydro-1,4-benzodioxinyl, pyridyl, thienyl or pyrazol-1-yl or pyrazol-2-yl,
   where the substituents are selected from cyano, fluorine, chlorine, bromine, iodine, SF₅, methyl, ethyl, n-propyl, i-propyl, cyclopropyl, fluoromethyl, difluoromethyl, trifluoromethyl, fluoroethyl, difluoroethyl, trifluoroethyl, tetrafluoroethyl, pentafluoroethyl, heptafluoroisopropyl, fluorocyclopropyl, difluorocyclopropyl, tetrafluorocyclopropyl, methoxy, ethoxy, trifluoromethoxy, difluoromethoxy, difluorochloromethoxy, dichlorofluoromethoxy, pentafluoroethoxy, tetrafluoroethoxy, trifluoroethoxy, chlorotetrafluoroethoxy, dichlorotrifluoroethoxy, trichlorodifluoroethoxy, chlorotrifluoroethoxy, chlorodifluoroethoxy, trifluoroethenoxy, trifluoromethylsulphonyl, difluoromethylsulphonyl, trifluoromethylsulphinyl, trifluoromethylsulphanyl, difluoromethylsulphinyl, difluoromethylsulphanyl, pentafluoroethylsulphanyl, trifluoroethylsulphanyl, difluoroethylsulphanyl, pentafluoroethylsulphonyl, tetrafluoroethylsulphonyl, trifluoroethylsulphonyl, difluoroethylsulphonyl, pentafluoroethylsulphinyl, tetrafluoroethylsulphinyl, trifluoroethylsulphinyl, difluoroethylsulphinyl, tetrafluoroethylsulphanyl, cyclopropylsulphanyl, trifluoromethylcyclopropyl, trifluoromethylcarbonyl, bis(trifluoromethyl)amino, (trifluorovinyl)oxy,
R¹ is methyl, ethyl, n-propyl, i-propyl, cyclopropyl, methylcyclopropyl, cyclopropylmethyl, cyanocyclopropyl, chlorocyclopropyl, fluorocyclopropyl, trifluoromethylcyclopropyl, cyclopropylcyclopropyl, methoxymethyl, methoxyethyl, n-butyl, i-butyl, tert-butyl, cyclobutyl, difluorocyclobutyl, cyclopentyl, fluoromethyl, difluoromethyl or trifluoromethyl,
   or is mono-phenyl-substituted methyl, ethyl, i-propyl or cyclopropyl, where phenyl in each case is optionally mono- or disubstituted identically or differently by fluorine, chlorine, bromine, cyano, nitro, methyl, ethyl, n-propyl, i-propyl, cyclopropyl, trifluoromethyl, methoxy, trifluoromethoxy,
   or is in each case optionally singly or doubly, identically or differently fluorine-, chlorine-, bromine-, cyano-, nitro-, methyl-, ethyl-, n-propyl-, i-propyl-, cyclopropyl-, trifluoromethyl-, methoxy-, trifluoromethoxy-substituted phenyl, pyridyl, oxazolyl, thiazolyl, thienyl, oxetanyl or thietanyl,
R² is hydrogen, methyl, ethyl, n-propyl, i-propyl, cyclopropyl, n-butyl, i-butyl, tert-butyl, cyclobutyl, methoxy, ethoxy, n-propoxy, i-propoxy, n-butoxy, i-butoxy, methylcarbonyl, ethylcarbonyl, n-propylcarbonyl, i-propylcarbonyl, cyclopropylcarbonyl, methoxycarbonyl, ethoxycarbonyl, n-propoxycarbonyl or i-propoxycarbonyl,
R³ is hydrogen, methyl, ethyl, n-propyl, i-propyl, cyclopropyl, n-butyl, i-butyl, tert-butyl, cyclobutyl, methoxy, ethoxy, n-propyloxy, i-propyloxy, n-butyloxy, i-butyloxy, methylcarbonyl, ethylcarbonyl, n-propylcarbonyl, i-propylcarbonyl, cyclopropylcarbonyl, methyloxycarbonyl, ethyloxycarbonyl, n-propyloxycarbonyl or i-propyloxycarbonyl,
R⁴ is methyl, ethyl, n-propyl, i-propyl, cyclopropyl, n-butyl, i-butyl, tert-butyl, cyclobutyl, fluoromethyl, difluoromethyl, trifluoromethyl, difluoroethyl, trifluoroethyl, pentafluoroethyl, trifluoropropyl, pentafluoropropyl, cyanomethyl, cyanoethyl, cyanopropyl, cyanobutyl, cyanopentyl, cyanohexyl, methyloxymethyl, ethyloxymethyl, methyloxyethyl, ethyloxyethyl, methyloxypropyl, ethyloxypropyl, propynyl, butynyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, cyclohexylethyl, cyanocyclopropylmethyl, cyanocyclobutylmethyl, cyanocyclopentylmethyl, cyanocyclohexylmethyl, trifluoromethylcyclopropylmethyl, trifluoromethylcyclohexylmethyl, cyclopropylcyclopropyl, methylcyclopropyl, ethylcyclopropyl, trifluoromethylcyclopropyl, trifluoromethylcyclobutyl, trifluoromethylcyclopentyl, trifluoromethylcyclohexyl, fluorocyclopropyl, difluorocyclopropyl, tetrafluorocyclopropyl, cyanocyclopropyl, cyanocyclobutyl, cyanocyclopentyl, cyanocyclohexyl, ethynylcyclopropyl, methyloxycyclopropyl, ethyloxycyclopropyl, methyloxycarbonylcyclopropyl, ethyloxycarbonylcyclopropyl, thiocarbamoylcyclopropyl, methyloxycarbonylmethyl, ethyloxycarbonylmethyl, methyloxycarbonylethyl, ethyloxycarbonylethyl, methylamino, ethylamino, n-propylamino, isopropylamino, cyclopropylamino, cyclobutylamino, cyclopentylamino, cyclohexylamino, benzylamino, cyanobenzylamino, nitrobenzylamino, dimethylamino, N-methylethylamino, N-methyl-n-propylamino, N-methyl-isopropylamino, N-methylcyclopropylamino, N-methylcyclobutylamino, N-methylcyclopentylamino, N-methyl-cyclohexylamino, N-methylbenzylamino, N-methylcyanobenzylamino, N-methylnitrobenzylamino, methoxy, ethoxy, n-propyloxy, isopropyloxy, cyclopropyloxy, cyclopropylmethyloxy, cyclobutyloxy, cyclopentyloxy, cyclohexyloxy, benzyloxy, cyanobenzyloxy, nitrobenzyloxy, methylimino, ethylimino, n-propylimino, isopropylimino, isobutylimino, cyclopentylimino, cyclohexylimino, benzylimino, cyanobenzylimino or nitrobenzylimino,
   or is mono-phenyl-, -pyridyl-, -pyrimidyl-, -furanyl-, -thiophenyl-, -oxazolyl-, -thiazolyl-, -indolyl-, -azaindolyl-, -indazolyl-, -azaindazolyl-, -1,3-benzodioxolyl- or -2,3-dihydro-1,4-benzodioxinyl-substituted methyl, ethyl, n-propyl, i-propyl, cyclopropyl, n-butyl, i-butyl, tert-butyl, cyclobutyl, cyclopentyl, cyclohexyl, oxy, amino, N-methylamino or N-ethylamino, where phenyl, pyridyl, pyrimidyl, furanyl, thiophenyl, oxazolyl, thiazolyl, indolyl, azaindolyl, indazolyl, azaindazolyl, 1,3-benzodioxolyl may each optionally be mono- or polysubstituted identically or differently by cyano, fluorine, chlorine, bromine, iodine, nitro, methyl, ethyl, n-propyl, i-propyl, cyclopropyl, trifluoromethyl, pentafluoroethyl, methoxy, ethoxy, trifluoromethoxy, pentafluoroethoxy, trifluoromethylthio, methyloxycarbonyl, ethyloxycarbonyl, carbamoyl, thiocarbamoyl, aminosulphonyl, pyrazolyl, imidazolyl, methylpyrazolyl, oxazolyl, oxdiazolyl or thiazolyl,
   or is in each case optionally mono- or disubstituted identically or differently by phenyl, pyridyl, pyrimidyl, thiophenyl, indazolyl, azaindazolyl, 1,3-benzodioxolyl, oxetanyl, thietanyl, tetrahydrofuranyl, oxotetrahydrofuranyl, tetrahydrothiophenyl, oxidotetrahydrothiophenyl, dioxidotetrahydrothiophenyl, tetrahydro-2H-pyranyl, oxotetrahydro-2H-pyranyl, tetrahydro-2H-thiopyranyl, oxotetrahydro-2H-thiopyranyl, 2,3-dihydro-1H-indenyl, where the substituents are selected from fluorine, chlorine, bromine, cyano, nitro, methyl, ethyl, n-propyl, i-propyl, cyclopropyl, trifluoromethyl, methoxy, trifluoromethoxy, trifluoromethylthio, methyloxycarbonyl or ethyloxycarbonyl,
      or R³ and R⁴ together are (C₃-C₅)-alkyl, forming a 4-6-membered ring optionally substituted by halogen, cyano, trifluoromethyl or trifluoromethoxy,
R⁵, R⁶ are independently hydrogen, cyano, fluorine, chlorine, bromine, methyl, ethyl, difluoromethyl, trifluoromethyl or trifluoromethoxy,
V¹, V² are independently oxygen or sulphur.

Alternative and likewise very particularly preferred compounds (Configuration 4-2) are those of the formula (I) in which
Q¹ is in each case optionally singly or multiply, identically or differently substituted phenyl, 1,3-benzodioxolyl, 2,3-dihydro-1,4-benzodioxinyl, pyridyl, thienyl or pyrazol-1-yl or pyrazol-2-yl,
   where the substituents are selected from cyano, fluorine, chlorine, bromine, iodine, SF₅, methyl, ethyl, n-propyl, i-propyl, cyclopropyl, fluoromethyl, difluoromethyl, trifluoromethyl, fluoroethyl, difluoroethyl, trifluoroethyl, tetrafluoroethyl, pentafluoroethyl, heptafluoroisopropyl, fluorocyclopropyl, difluorocyclopropyl, tetrafluorocyclopropyl, methoxy, ethoxy, trifluoromethoxy, difluoromethoxy, difluorochloromethoxy, dichlorofluoromethoxy, pentafluoroethoxy, tetrafluoroethoxy, trifluoroethoxy, chlorotetrafluoroethoxy, dichlorotrifluoroethoxy, trichlorodifluoroethoxy, chlorotrifluoroethoxy, chlorodifluoroethoxy, trifluoroethenoxy, trifluoromethylsulphonyl, difluoromethylsulphonyl, trifluoromethylsulphinyl, trifluoromethylsulphanyl, difluoromethylsulphinyl, difluoromethylsulphanyl, pentafluoroethylsulphanyl, trifluoroethylsulphanyl, difluoroethylsulphanyl, pentafluoroethylsulphonyl, tetrafluoroethylsulphonyl, trifluoroethylsulphonyl, difluoroethylsulphonyl, pentafluoroethylsulphinyl, tetrafluoroethylsulphinyl, trifluoroethylsulphinyl, difluoroethylsulphinyl, tetrafluoroethylsulphanyl, cyclopropylsulphanyl, trifluoromethylcyclopropyl, trifluoromethylcarbonyl, bis(trifluoromethyl)amino, (trifluorovinyl)oxy, heptafluororopoxy or hexafluoropropoxy,
R¹ is methyl, ethyl, n-propyl, i-propyl, cyclopropyl, methylcyclopropyl, cyclopropylmethyl, cyanocyclopropyl, chlorocyclopropyl, fluorocyclopropyl, trifluoromethylcyclopropyl, cyclopropylcyclopropyl, methoxymethyl, methoxyethyl, n-butyl, i-butyl, tert-butyl, cyclobutyl, difluorocyclobutyl, cyclopentyl, fluoromethyl, difluoromethyl, trifluoromethyl or trifluoroethyl, or is mono-phenyl-substituted methyl, ethyl, i-propyl or cyclopropyl, where phenyl in each case is optionally mono- or disubstituted identically or differently by fluorine, chlorine, bromine, cyano, nitro, methyl, ethyl, n-propyl, i-propyl, cyclopropyl, trifluoromethyl, methoxy, trifluoromethoxy,
   or is in each case optionally singly or doubly, identically or differently fluorine-, chlorine-, bromine-, cyano-, nitro-, methyl-, ethyl-, n-propyl-, i-propyl-, cyclopropyl-, trifluoromethyl-, methoxy-, trifluoromethoxy-substituted phenyl, pyridyl, oxazolyl, thiazolyl, thienyl, oxetanyl or thietanyl,
R² is hydrogen, methyl, ethyl, n-propyl, i-propyl, cyclopropyl, n-butyl, i-butyl, tert-butyl, cyclobutyl, methoxy, ethoxy, n-propoxy, i-propoxy, n-butoxy, i-butoxy, methylcarbonyl, ethylcarbonyl, n-propylcarbonyl, i-propylcarbonyl, cyclopropylcarbonyl, methoxycarbonyl, ethoxycarbonyl, n-propoxycarbonyl or i-propoxycarbonyl,
R³ is hydrogen, methyl, ethyl, n-propyl, i-propyl, cyclopropyl, n-butyl, i-butyl, tert-butyl, cyclobutyl, methoxy, ethoxy, n-propyloxy, i-propyloxy, n-butyloxy, i-butyloxy, methylcarbonyl, ethylcarbonyl, n-propylcarbonyl, i-propylcarbonyl, cyclopropylcarbonyl, methyloxycarbonyl, ethyloxycarbonyl, n-propyloxycarbonyl or i-propyloxycarbonyl,
R⁴ is methyl, ethyl, n-propyl, i-propyl, cyclopropyl, n-butyl, i-butyl, tert-butyl, cyclobutyl, fluoromethyl, difluoromethyl, trifluoromethyl, difluoroethyl, trifluoroethyl, pentafluoroethyl, trifluoropropyl, pentafluoropropyl, cyanomethyl, cyanoethyl, cyanopropyl, cyanobutyl, cyanopentyl, cyanohexyl, methyloxymethyl, ethyloxymethyl, methyloxyethyl, ethyloxyethyl, methyloxypropyl, ethyloxypropyl, propynyl, butynyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, cyclohexylethyl, cyanocyclopropylmethyl, cyanocyclobutylmethyl, cyanocyclopentylmethyl, cyanocyclohexylmethyl, trifluoromethylcyclopropylmethyl, trifluoromethylcyclohexylmethyl, cyclopropylcyclopropyl, methylcyclopropyl, ethylcyclopropyl, trifluoromethylcyclopropyl, trifluoromethylcyclobutyl, trifluoromethylcyclopentyl, trifluoromethylcyclohexyl, fluorocyclopropyl, difluorocyclopropyl, tetrafluorocyclopropyl, cyanocyclopropyl, cyanocyclobutyl, cyanocyclopentyl, cyanocyclohexyl, ethynylcyclopropyl, methyloxycyclopropyl, ethyloxycyclopropyl, methyloxycarbonylcyclopropyl, ethyloxycarbonylcyclopropyl, thiocarbamoylcyclopropyl, methyloxycarbonylmethyl, ethyloxycarbonylmethyl, methyloxycarbonylethyl, ethyloxycarbonylethyl, methylamino, ethylamino, n-propylamino, isopropylamino, cyclopropylamino, cyclobutylamino, cyclopentylamino, cyclohexylamino, benzylamino, cyanobenzylamino, nitrobenzylamino, dimethylamino, N-methylethylamino, N-methyl-n-propylamino, N-methyl-isopropylamino, N-methylcyclopropylamino, N-methylcyclobutylamino, N-methylcyclopentylamino, N-methyl-cyclohexylamino, N-methylbenzylamino, N-methylcyanobenzylamino, N-methylnitrobenzylamino, methoxy, ethoxy, n-propyloxy, isopropyloxy, cyclopropyloxy, cyclopropylmethyloxy, cyclobutyloxy, cyclopentyloxy, cyclohexyloxy, cyanomethyloxy, cyanoethyloxy, cyanopropyloxy, cyanobutyloxy, benzyloxy, cyanobenzyloxy, nitrobenzyloxy, methylimino, ethylimino, n-propylimino, isopropylimino, isobutylimino, cyclopentylimino, cyclohexylimino, benzylimino, cyanobenzylimino, nitrobenzylimino, fluorobenzylimino, chlorobenzylimino, trifluoromethylbenzylimino, chloro(trifluoromethyl)benzylimino, fluoro(trifluoromethyl)benzylimino, methylsulphonylmethyl, methylsulphonylethyl, methylsulphonylpropyl, methylsulphonylbutyl, methylcarbonyl, ethylcarbonyl, propylcarbonyl, isopropylcarbonyl, cyclopropylcarbonyl, cyclopropylmethylcarbonyl, cyclobutylmethylcarbonyl, butylcarbonyl, isobutylcarbonyl, tert-butylcarbonyl, cyclobutylcarbonyl, cyclopentylcarbonyl, cyclohexylcarbonyl or trifluoromethylcarbonyl,
   or is mono-phenyl-, -pyridyl-, -pyrimidyl-, -furanyl-, -thiophenyl-, -oxazolyl-, -thiazolyl-, -indolyl-, -azaindolyl-, -indazolyl-, -azaindazolyl-, -1,3-benzodioxolyl- or -2,3-dihydro-1,4-benzodioxinyl-substituted methyl, ethyl, n-propyl, i-propyl, cyclopropyl, n-butyl, i-butyl, tert-butyl, cyclobutyl, cyclopentyl, cyclohexyl, oxy, amino, N-methylamino, N-ethylamino or carbonyl, where phenyl, pyridyl, pyrimidyl, furanyl, thiophenyl, oxazolyl, thiazolyl, indolyl, azaindolyl, indazolyl, azaindazolyl, 1,3-benzodioxolyl may each optionally be mono- or polysubstituted identically or differently by cyano, fluorine, chlorine, bromine, iodine, nitro, methyl, ethyl, n-propyl, i-propyl, cyclopropyl, trifluoromethyl, pentafluoroethyl, methoxy, ethoxy, trifluoromethoxy, pentafluoroethoxy, trifluoromethylthio, methyloxycarbonyl, ethyloxycarbonyl, carbamoyl, thiocarbamoyl, aminosulphonyl, pyrazolyl, imidazolyl, methylpyrazolyl, oxazolyl, oxdiazolyl, thiazolyl, pyrrolyl, pyrrolidinyl or pyrrolidinonyl,
   or is in each case optionally mono- or disubstituted identically or differently by phenyl, pyridyl, pyrimidyl, thiophenyl, indazolyl, azaindazolyl, 1,3-benzodioxolyl, oxetanyl, thietanyl, tetrahydrofuranyl, oxotetrahydrofuranyl, tetrahydrothiophenyl, oxidotetrahydrothiophenyl, dioxidotetrahydrothiophenyl, tetrahydro-2H-pyranyl, oxotetrahydro-2H-pyranyl, tetrahydro-2H-thiopyranyl, oxotetrahydro-2H-thiopyranyl, 2,3-dihydro-1H-indenyl, where the substituents are selected from fluorine, chlorine, bromine, cyano, nitro, methyl, ethyl, n-propyl, i-propyl, cyclopropyl, trifluoromethyl, methoxy, trifluoromethoxy, trifluoromethylthio, methyloxycarbonyl or ethyloxycarbonyl,
      or R³ and R⁴ together are (C₃-C₅)-alkyl, forming a 4-6-membered ring optionally substituted by halogen, cyano, trifluoromethyl or trifluoromethoxy,
R⁵, R⁶ are independently hydrogen, cyano, fluorine, chlorine, bromine, methyl, ethyl, difluoromethyl, trifluoromethyl or trifluoromethoxy,
V¹ is oxygen or sulphur,
V² is oxygen, sulphur or —NH.

### Very Especially Preferred Range:

Very especially preferred compounds (Configuration 5-1) are those of the formula (I) in which
Q¹ is in each case optionally singly, doubly, triply or quadruply, identically or differently substituted phenyl, 1,3-benzodioxolyl, 2,3-dihydro-1,4-benzodioxinyl, pyridyl, thienyl or pyrazol-1-yl or pyrazol-2-yl,
   where the substituents are selected from fluorine, chlorine, bromine, iodine, SF₅, methyl, ethyl, difluoromethyl, trifluoromethyl, difluoroethyl, trifluoroethyl, tetrafluoroethyl, pentafluoroethyl, heptafluoroisopropyl, trifluoromethoxy, difluoromethoxy, difluorochloromethoxy, pentafluoroethoxy, tetrafluoroethoxy, chlorotrifluoroethoxy, trifluoroethenoxy, trifluoromethylsulphonyl, trifluoromethylsulphinyl, trifluoromethylsulphanyl, difluoromethylsulphinyl, difluoromethylsulphanyl, trifluoromethylcyclopropyl, bis(trifluoromethyl)amino, tetrafluoroethylsulphanyl, cyclopropylsulphanyl, (trifluorovinyl)oxy,
R¹ is methyl, ethyl, i-propyl, cyclopropyl, methylcyclopropyl, cyclopropylmethyl, cyanocyclopropyl, chlorocyclopropyl, cyclopropylcyclopropyl, methoxyethyl, tert-butyl, cyclobutyl, difluorocyclobutyl or cyclopentyl,
   or mono-phenyl-substituted methyl, where phenyl may optionally be monosubstituted by chlorine, bromine, nitro, trifluoromethyl,
   or is optionally mono-chlorine-substituted phenyl or is oxazolyl,
R² is hydrogen, methyl, ethyl, cyclopropylcarbonyl or methoxycarbonyl,
R³ is hydrogen, methyl, methoxy or methoxycarbonyl,
R⁴ is methyl, ethyl, n-propyl, i-propyl, cyclopropyl, n-butyl, i-butyl, cyclobutyl, trifluoroethyl, trifluoropropyl, cyanomethyl, cyanoethyl, cyanopropyl, cyanobutyl, cyanopentyl, methyloxyethyl, propynyl, butynyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyanocyclohexylmethyl, trifluoromethylcyclohexylmethyl, cyclopropylcyclopropyl, methylcyclopropyl, trifluoromethylcyclopropyl, trifluoromethylcyclohexyl, difluorocyclopropyl, cyanocyclopropyl, cyanocyclobutyl, cyanocyclohexyl, ethynylcyclopropyl, methyloxycyclopropyl, methyloxycarbonylcyclopropyl, thiocarbamoylcyclopropyl or methyloxy carbonylethyl,
   or is mono-phenyl-, -pyridyl-, -indazolyl-, -azaindazolyl- or -1,3-benzodioxolyl-substituted methyl, ethyl, n-propyl or cyclopropyl, where phenyl, pyridyl, indazolyl, azaindazolyl or 1,3-benzodioxolyl may each optionally be mono-, di- or trisubstituted identically or differently by cyano, fluorine, chlorine, nitro, methyl, trifluoromethyl, methoxy, methyloxycarbonyl, thiocarbamoyl, aminosulphonyl, methylpyrazolyl, oxazolyl or oxadiazolyl,
   or is in each case optionally substituted phenyl, pyridyl, indazolyl, oxetanyl, thietanyl, tetrahydrofuranyl, oxotetrahydrofuranyl, dioxidotetrahydrothiophenyl, tetrahydro-2H-pyranyl or 2,3-dihydro-1H-indenyl, where the substituents are selected from chlorine, cyano, methyl and methyloxycarbonyl,
      or R³ and R⁴ together are (C₃-C₅)-alkyl, forming a 4-6-membered ring optionally substituted by cyano,
R⁵, R⁶ are independently hydrogen,
V¹, V² are independently oxygen or sulphur.

Alternative and likewise very especially preferred compounds (Configuration 5-2) are those of the formula (I) in which
Q¹ is in each case optionally singly, doubly, triply or quadruply, identically or differently substituted phenyl, 1,3-benzodioxolyl, 2,3-dihydro-1,4-benzodioxinyl, pyridyl, thienyl or pyrazol-1-yl or pyrazol-2-yl,
   where the substituents are selected from fluorine, chlorine, bromine, iodine, SF₅, methyl, ethyl, difluoromethyl, trifluoromethyl, difluoroethyl, trifluoroethyl, tetrafluoroethyl, pentafluoroethyl, heptafluoroisopropyl, trifluoromethoxy, difluoromethoxy, difluorochloromethoxy, pentafluoroethoxy, tetrafluoroethoxy, chlorotrifluoroethoxy, trifluoroethenoxy, trifluoromethylsulphonyl, trifluoromethylsulphinyl, trifluoromethylsulphanyl, difluoromethylsulphinyl, difluoromethylsulphanyl, trifluoromethylcyclopropyl, bis(trifluoromethyl)amino, tetrafluoroethylsulphanyl, cyclopropylsulphanyl, (trifluorovinyl)oxy and hexafluoropropoxy,
R¹ is methyl, ethyl, i-propyl, cyclopropyl, methylcyclopropyl, cyclopropylmethyl, cyanocyclopropyl, chlorocyclopropyl, cyclopropylcyclopropyl, methoxyethyl, tert-butyl, cyclobutyl, difluorocyclobutyl, cyclopentyl, trifluoroethyl or trifluoromethylcyclopropyl,
   or mono-phenyl-substituted methyl, where phenyl may optionally be monosubstituted by chlorine, bromine, nitro, trifluoromethyl,
   is optionally mono-chlorine-substituted phenyl or pyridyl or is oxazolyl,
R² is hydrogen, methyl, ethyl, cyclopropylcarbonyl or methoxycarbonyl,
R³ is hydrogen, methyl, methoxy or methoxycarbonyl,
R⁴ is methyl, ethyl, n-propyl, i-propyl, cyclopropyl, n-butyl, i-butyl, cyclobutyl, trifluoroethyl, trifluoropropyl, cyanomethyl, cyanoethyl, cyanopropyl, cyanobutyl, cyanopentyl, methyloxyethyl, propynyl, butynyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyanocyclohexylmethyl, trifluoromethylcyclohexylmethyl, cyclopropylcyclopropyl, methylcyclopropyl, trifluoromethylcyclopropyl, trifluoromethylcyclohexyl, difluorocyclopropyl, cyanocyclopropyl, cyanocyclobutyl, cyanocyclohexyl, ethynylcyclopropyl, methyloxycyclopropyl, methyloxycarbonylcyclopropyl, thiocarbamoylcyclopropyl, methyloxycarbonylethyl, cyanobenzylimino, fluoro(trifluoromethyl)benzylimino, methylsulphonylbutyl, methoxy, ethoxy, cyclopropylmethyloxy, isopropyloxy, cyanopropyloxy, cyanophenyloxy, cyclopropylcarbonyl,
   or is mono-phenyl-, -pyridyl-, -indazolyl-, -azaindazolyl-, -thiophenyl- or -1,3-benzodioxolyl-substituted methyl, ethyl, n-propyl, cyclopropyl or carbonyl, where phenyl, pyridyl, indazolyl, azaindazolyl, thiophenyl or 1,3-benzodioxolyl may each optionally be mono-, di- or trisubstituted identically or differently by cyano, fluorine, chlorine, nitro, methyl, trifluoromethyl, methoxy, methyloxycarbonyl, thiocarbamoyl, aminosulphonyl, methylpyrazolyl, oxazolyl, oxadiazolyl or pyrrolidinonyl,
   or is in each case optionally substituted phenyl, pyridyl, indazolyl, oxetanyl, thietanyl, tetrahydrofuranyl, oxotetrahydrofuranyl, dioxidotetrahydrothiophenyl, tetrahydro-2H-pyranyl or 2,3-dihydro-1H-indenyl, where the substituents are selected from chlorine, cyano, methyl and methyloxycarbonyl,
      or R³ and R⁴ together are (C₃-C₅)-alkyl, forming a 4-6-membered ring optionally substituted by cyano,
R⁵, R⁶ are independently hydrogen,
V¹ is oxygen or sulphur,
V² is oxygen, sulphur or —NH.

### Most Preferred Range:

Most preferred compounds (Configuration 6-1) are those of the formula (I) in which
Q¹ is 2,2,3,3-tetrafluoro-2,3-dihydro-1,4-benzodioxin-6-yl, 2,2-difluoro-1,3-benzodioxol-5-yl, 2-chloro-4-(trifluoromethoxy)phenyl, 2-ethyl-6-methyl-4-(pentafluoroethyl)phenyl, 2-fluoro-4-(trifluoromethoxy)phenyl, 2-fluoro-6-(trifluoromethyl)pyridin-3-yl, 2-methyl-4-(trifluoromethoxy)phenyl, 3-(pentafluoroethyl)-1H-pyrazol-1-yl, 3-(trifluoromethoxy)phenyl, 3,5-difluoro-4-(trifluoromethoxy)phenyl, 3-chloro-4-(trifluoromethoxy)phenyl, 3-chloro-5-(trifluoromethyl)pyridin-2-yl, 3-fluoro-4-(pentafluoroethyl)phenyl, 3-fluoro-4-(trifluoromethoxy)phenyl, 3-fluoro-4-(trifluoromethyl)phenyl, 3-methyl-4-(trifluoromethoxy)phenyl, 4-(1,1,1,2,3,3,3-heptafluoropropan-2-yl)phenyl, 4-(1,1,2,2-tetrafluoroethoxy)phenyl, 4-(1,1-difluoroethyl)phenyl, 4-(2,2,2-trifluoroethyl)phenyl, 4-(2-chloro-1,1,2-trifluoroethoxy)phenyl, 4-(cyclopropylsulphanyl)phenyl, 4-(difluoromethoxy)-3,5-difluorophenyl, 4-(difluoromethoxy)phenyl, 4-(difluoromethyl)-3-fluorophenyl, 4-(pentafluoroethoxy)phenyl, 4-(pentafluoroethyl)phenyl, 4-SF₅-phenyl, 4-(trifluoromethoxy)phenyl, 4-(trifluoromethyl)phenyl, 4-[(1,1,2,2-tetrafluoroethyl)sulphanyl]phenyl, 4-[(difluoromethyl)sulphanyl]phenyl, 4-[(difluoromethyl)sulphinyl]phenyl, 4-[(trifluoromethyl)sulphanyl]phenyl, 4-[(trifluoromethyl)sulphinyl]phenyl, 4-[(trifluoromethyl)sulphonyl]phenyl, 4-[(trifluorovinyl)oxy]phenyl, 4-[1-(trifluoromethyl)cyclopropyl]phenyl, 4-[bis(trifluoromethyl)amino]phenyl, 4-[chloro(difluoro)methoxy]phenyl, 4-bromophenyl,4-chlorophenyl, 4-fluorophenyl, 4-iodophenyl, 5-(trifluoromethyl)-2-thienyl, 5-(trifluoromethyl)pyridin-2-yl, 6-(trifluoromethyl)pyridin-3-yl,
R¹ is cyclobutyl, cyclopentyl, cyclopropyl, cyclopropylmethyl, ethyl, isopropyl, methyl, tert-butyl, 1,3-oxazol-5-yl, 1-chlorocyclopropyl, 1-cyanocyclopropyl, 1-cyclopropylcyclopropyl, 1-methoxyethyl, 1-methylcyclopropyl, 2-chlorophenyl, 3,3-difluorocyclobutyl, 4-(trifluoromethyl)benzyl, 4-bromobenzyl, 4-chlorophenyl or 4-nitrobenzyl,
R² is hydrogen, methyl, ethyl, cyclopropylcarbonyl or methoxycarbonyl,
R³ is hydrogen, methyl, methoxy or methoxycarbonyl,
R⁴ is (1-methyl-1H-indazol-7-yl)methyl, (1-methyl-1H-pyrazolo[3,4-b]pyridin-5-yl)methyl, (1S)-1-[4-(trifluoromethyl)phenyl]ethyl, (1S)-1-[4-(trifluoromethyl)phenyl]ethyl, (2,2-difluoro-1,3-benzodioxol-5-yl)methyl, (4-cyanocyclohexyl)methyl, (5-cyanopyridin-2-yl)methyl, (6-chloropyridin-3-yl)methyl, (6-cyanopyridin-3-yl)methyl, (trans-4-cyanocyclohexyl)methyl, [cis-4-(trifluoromethyl)cyclohexyl]methyl, 1-(4-cyanophenyl)cyclopropyl, 1-(4-cyanophenyl)ethyl, 1-(4-nitrophenyl)cyclopropyl, 1-(4-nitrophenyl)ethyl, 1-(methoxycarbonyl)cyclopropyl, 1-(trifluoromethyl)cyclopropyl, 1,1-dioxidotetrahydrothiophen-3-yl, 1-[4-(trifluoromethyl)phenyl]ethyl, 1-thiocarbamoylcyclopropyl, 1-cyanocyclobutyl, 1-cyanocyclopropyl, 1-cyanoethyl, 1-cyclopropylcyclopropyl, 1-cyclopropylethyl, 1-ethynylcyclopropyl, 1-methoxycarbonylethyl, 1-methoxycyclopropyl, 1-methylcyclopropyl, 2-(3-cyanophenyl)ethyl, 2-(4-cyanophenyl)ethyl, 2,2,2-trifluoroethyl, 2,2-difluorocyclopropyl, 2,4-dichloro-5-fluorobenzyl, 2,4-dichlorobenzyl, 2-cyanopropan-2-yl, 2-fluoro-4-(trifluoromethyl)benzyl, 2-fluoro-4-nitrobenzyl, 2-methoxyethyl, 2-oxotetrahydrofuran-3-yl, 3-(methoxycarbonyl)oxetan-3-yl, 3,3,3-trifluoropropyl, 3-[4-(trifluoromethyl)phenyl]propyl, 3-chlorobenzyl, 3-cyanobenzyl, 3-cyanopentan-3-yl, 3-fluoro-4-(trifluorbenzyl)benzyl, 3-fluorobenzyl, 3-methylthietan-3-yl, 4-(1,2,4-oxadiazol-3-yl)benzyl, 4-(1,3,4-oxadiazol-2-yl)benzyl, 4-(1,3-oxazol-5-yl)benzyl, 4-(1-methyl-1H-pyrazol-3-yl)benzyl, 4-(1-methyl-1H-pyrazol-5-yl)benzyl, 4-(methoxycarbonyl)benzyl, 4-(trifluoromethyl)benzyl, 4-thiocarbamoylbenzyl, 4-chloro-2-fluorobenzyl, 4-chloro-3-fluorbenzyl, 4-chlorobenzyl, 4-chlorophenyl, 4-cyano-2,5-difluorobenzyl, 4-cyano-2,6-difluorobenzyl, 4-cyano-2-fluorobenzyl, 4-cyano-3,5-difluorobenzyl, 4-cyano-3-fluorobenzyl, 4-cyanobenzyl, 4-cyanobutyl, 4-cyanocyclohexyl, 4-cyanophenyl, 4-cyanotetrahydro-2H-pyran-4-yl, 4-fluoro-3-(trifluoromethyl)benzyl, 4-methoxybenzyl, 4-nitrobenzyl, 4-aminosulphonylbenzyl, 5-cyano-1H-indazol-3-yl, 5-cyano-2,3-dihydro-1H-inden-1-yl, 5-cyanopentyl, benzyl, but-3-yn-2-yl, n-butyl, cis-4-(trifluoromethyl)cyclohexyl, cyanomethyl, cyclobutyl, cyclobutylmethyl, cyclohexyl, cyclohexylmethyl, cyclopentyl, cyclopentylmethyl, cyclopropyl, cyclopropylmethyl, ethyl, isobutyl, isopropyl, methyl, oxetan-3-yl, prop-2-yn-1-yl, n-propyl, pyridin-2-ylmethyl, pyridin-3-yl, thietan-3-yl or trans-4-(trifluoromethyl)cyclohexyl,
   or R³ and R⁴ together are one of the following rings:

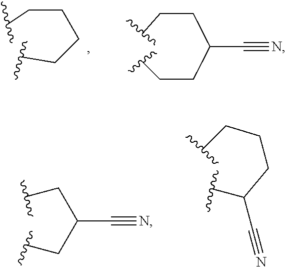 [SMILES: *CCCC* ; *CCC(C#N)CC* ; *CC(C#N)C* ; *CCCC(*)C#N]

R⁵, R⁶ are independently hydrogen,
V¹, V² are independently oxygen or sulphur.

Alternative and likewise most preferred compounds (Configuration 6-2) are those of the formula (I) in which
Q¹ is 2,2,3,3-tetrafluoro-2,3-dihydro-1,4-benzodioxin-6-yl, 2,2-difluoro-1,3-benzodioxol-5-yl, 2-chloro-4-(trifluoromethoxy)phenyl, 2-ethyl-6-methyl-4-(pentafluoroethyl)phenyl, 2-fluoro-4-(trifluoromethoxy)phenyl, 2-fluoro-6-(trifluoromethyl)pyridin-3-yl, 2-methyl-4-(trifluoromethoxy)phenyl, 3-(pentafluoroethyl)-1H-pyrazol-1-yl, 3-(trifluoromethoxy)phenyl, 3,5-difluoro-4-(trifluoromethoxy)phenyl, 3-chloro-4-(trifluoromethoxy)phenyl, 3-chloro-5-(trifluoromethyl)pyridin-2-yl, 3-fluoro-4-(pentafluoroethyl)phenyl, 3-fluoro-4-(trifluoromethoxy)phenyl, 3-fluoro-4-(trifluoromethyl)phenyl, 3-methyl-4-(trifluoromethoxy)phenyl, 4-(1,1,1,2,3,3,3-heptafluoropropan-2-yl)phenyl, 4-(1,1,2,2-tetrafluoroethoxy)phenyl, 4-(1,1-difluoroethyl)phenyl, 4-(2,2,2-trifluoroethyl)phenyl, 4-(2-chloro-1,1,2-trifluoroethoxy)phenyl, 4-(cyclopropylsulphanyl)phenyl, 4-(difluoromethoxy)-3,5-difluorophenyl, 4-(difluoromethoxy)phenyl, 4-(difluoromethyl)-3-fluorophenyl, 4-(pentafluoroethoxy)phenyl, 4-(pentafluoroethyl)phenyl, 4-SF₅-phenyl, 4-(trifluoromethoxy)phenyl, 4-(trifluoromethyl)phenyl, 4-[(1,1,2,2-tetrafluoroethyl)sulphanyl]phenyl, 4-[(difluoromethyl)sulphanyl]phenyl, 4-[(difluoromethyl)sulphinyl]phenyl, 4-[(trifluoromethyl)sulphanyl]phenyl, 4-[(trifluoromethyl)sulphinyl]phenyl, 4-[(trifluoromethyl)sulphonyl]phenyl, 4-[(trifluorovinyl)oxy]phenyl, 4-[1-(trifluoromethyl)cyclopropyl]phenyl, 4-[bis(trifluoromethyl)amino]phenyl, 4-[chloro(difluoro)methoxy]phenyl, 4-bromophenyl,4-chlorophenyl, 4-fluorophenyl, 4-iodophenyl, 5-(trifluoromethyl)-2-thienyl, 5-(trifluoromethyl)pyridin-2-yl, 6-(trifluoromethyl)pyridin-3-yl, 4-bromo-3-fluorophenyl, 4-(1,1,2,3,3,3-hexafluoropropoxy)phenyl, 2,6-dichloro-4-(pentafluoroethyl)phenyl, 2-chloro-3-fluoro-4-(pentafluoroethyl)phenyl, 3-fluoro-5-methyl-4-(pentafluoroethyl)phenyl, 2,6-difluoro-4-(pentafluoroethyl)phenyl, 2-chloro-5-fluoro-4-(pentafluoroethyl)phenyl, 3-methyl-4-(pentafluoroethyl)phenyl, 3,5-difluoro-4-(pentafluoroethyl)phenyl, 2,3-difluoro-4-(pentafluoroethyl)phenyl, 3-chloro-4-(pentafluoroethyl)phenyl, 2,3,5-trifluoro-4-(pentafluoroethyl)phenyl, 2,5-difluoro-4-(pentafluoroethyl)phenyl, 2,6-dichloro-3-fluoro-4-(pentafluoroethyl)phenyl or 3-chloro-5-fluoro-4-(pentafluoroethyl)phenyl,
R¹ is cyclobutyl, cyclopentyl, cyclopropyl, cyclopropylmethyl, ethyl, isopropyl, methyl, tert-butyl, 1,3-oxazol-5-yl, 1-chlorocyclopropyl, 1-cyanocyclopropyl, 1-cyclopropylcyclopropyl, 1-methoxyethyl, 1-methylcyclopropyl, 2-chlorophenyl, 3,3-difluorocyclobutyl, 4-(trifluoromethyl)benzyl, 4-bromobenzyl, 4-chlorophenyl, 4-nitrobenzyl, 2,2,2-trifluoroethyl, 1-(trifluoromethyl)cyclopropyl or 6-chloropyridin-3-yl,
R² is hydrogen, methyl, ethyl, cyclopropylcarbonyl or methoxycarbonyl,
R³ is hydrogen, methyl, methoxy or methoxycarbonyl,
R⁴ is (1-methyl-1H-indazol-7-yl)methyl, (1-methyl-1H-pyrazolo[3,4-b]pyridin-5-yl)methyl, (1S)-1-[4-(trifluoromethyl)phenyl]ethyl, (1S)-1-[4-(trifluoromethyl)phenyl]ethyl, (2,2-difluoro-1,3-benzodioxol-5-yl)methyl, (4-cyanocyclohexyl)methyl, (5-cyanopyridin-2-yl)methyl, (6-chloropyridin-3-yl)methyl, (6-cyanopyridin-3-yl)methyl, (trans-4-cyanocyclohexyl)methyl, [cis-4-(trifluoromethyl)cyclohexyl]methyl, 1-(4-cyanophenyl)cyclopropyl, 1-(4-cyanophenyl)ethyl, 1-(4-nitrophenyl)cyclopropyl, 1-(4-nitrophenyl)ethyl, 1-(methoxycarbonyl)cyclopropyl, 1-(trifluoromethyl)cyclopropyl, 1,1-dioxidotetrahydrothiophen-3-yl, 1-[4-(trifluoromethyl)phenyl]ethyl, 1-thiocarbamoylcyclopropyl, 1-cyanocyclobutyl, 1-cyanocyclopropyl, 1-cyanoethyl, 1-cyclopropylcyclopropyl, 1-cyclopropylethyl, 1-ethynylcyclopropyl, 1-methoxycarbonylethyl, 1-methoxycyclopropyl, 1-methylcyclopropyl, 2-(3-cyanophenyl)ethyl, 2-(4-cyanophenyl)ethyl, 2,2,2-trifluoroethyl, 2,2-difluorocyclopropyl, 2,4-dichloro-5-fluorobenzyl, 2,4-dichlorobenzyl, 2-cyanopropan-2-yl, 2-fluoro-4-(trifluoromethyl)benzyl, 2-fluoro-4-nitrobenzyl, 2-methoxyethyl, 2-oxotetrahydrofuran-3-yl, 3-(methoxycarbonyl)oxetan-3-yl, 3,3,3-trifluoropropyl, 3-[4-(trifluoromethyl)phenyl]propyl, 3-chlorobenzyl, 3-cyanobenzyl, 3-cyanopentan-3-yl, 3-fluoro-4-(trifluorobenzyl)benzyl, 3-fluorobenzyl, 3-methylthietan-3-yl, 4-(1,2,4-oxadiazol-3-yl)benzyl, 4-(1,3,4-oxadiazol-2-yl)benzyl, 4-(1,3-oxazol-5-yl)benzyl, 4-(1-methyl-1H-pyrazol-3-yl)benzyl, 4-(1-methyl-1H-pyrazol-5-yl)benzyl, 4-(methoxycarbonyl)benzyl, 4-(trifluoromethyl)benzyl, 4-thiocarbamoylbenzyl, 4-chloro-2-fluorobenzyl, 4-chloro-3-fluorobenzyl, 4-chlorobenzyl, 4-chlorophenyl, 4-cyano-2,5-difluorobenzyl, 4-cyano-2,6-difluorobenzyl, 4-cyano-2-fluorobenzyl, 4-cyano-3,5-difluorobenzyl, 4-cyano-3-fluorobenzyl, 4-cyanobenzyl, 4-cyanobutyl, 4-cyanocyclohexyl, 4-cyanophenyl, 4-cyanotetrahydro-2H-pyran-4-yl, 4-fluoro-3-(trifluoromethyl)benzyl, 4-methoxybenzyl, 4-nitrobenzyl, 4-aminosulphonylbenzyl, 5-cyano-1H-indazol-3-yl, 5-cyano-2,3-dihydro-1H-inden-1-yl, 5-cyanopentyl, benzyl, but-3-yn-2-yl, n-butyl, cis-4-(trifluoromethyl)cyclohexyl, cyanomethyl, cyclobutyl, cyclobutylmethyl, cyclohexyl, cyclohexylmethyl, cyclopentyl, cyclopentylmethyl, cyclopropyl, cyclopropylmethyl, ethyl, isobutyl, isopropyl, methyl, oxetan-3-yl, prop-2-yn-1-yl, n-propyl, pyridin-2-ylmethyl, pyridin-3-yl, thietan-3-yl, trans-4-(trifluoromethyl)cyclohexyl, (5-nitro-2-thienyl)methyl, (5-cyano-2-thienyl)methyl, 4-cyanobenzylimine, 3-fluoro-4-(trifluoromethyl)benzylimine, [4-(2-oxopyrrolidin-1-yl)phenyl]methyl, 2-methyl-1-methylsulphonylpropan-2-yl, (pyridin-4-yl)methyl, methoxy, ethoxy, cyclopropylmethyloxy, isopropyloxy, 3-cyanopropyloxy, 4-cyanophenyloxy, cyclopropylcarbonyl or 4-nitrobenzoyl,
   or R³ and R⁴ together are one of the following rings:

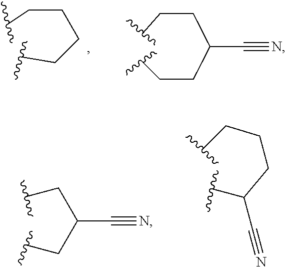 [SMILES: *CCCC* ; *CCC(C#N)CC* ; *CC(C#N)C* ; *CCCC(*)C#N]

R⁵, R⁶ are independently hydrogen,
V¹ is oxygen or sulphur,
V² is oxygen, sulphur or —NH.

### Further Preferred, Particularly Preferred and Very Particularly Preferred Ranges:

In a further preferred embodiment, the invention relates to the compounds of the general formula (I-a)

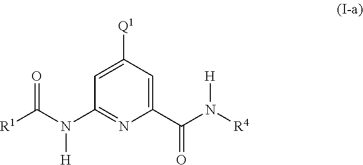

where R¹, R⁴ and Q¹ have the definitions described above (R⁵ and R⁶ are hydrogen), especially the definitions described in Configuration (1-1) or Configuration (2-1) or Configuration (3-1) or Configuration (4-1) or Configuration (5-1) or Configuration (6-1) or the definitions described in Configuration (1-2) or Configuration (2-2) or Configuration (3-2) or Configuration (4-2) or Configuration (5-2) or Configuration (6-2).

In a further preferred embodiment, the invention relates to the compounds of the general formula (I-b)

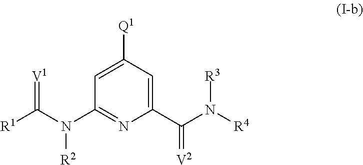

where R¹, R², R³, R⁴, V¹ and V² have the definitions described above (R⁵ and R⁶ are hydrogen), especially the definitions described in Configuration (1-1) or Configuration (2-1) or Configuration (3-1) or Configuration (4-1) or Configuration (5-1) or Configuration (6-1) or the definitions described in Configuration (1-2) or Configuration (2-2) or Configuration (3-2) or Configuration (4-2) or Configuration (5-2) or Configuration (6-2),
and Q¹ is optionally singly, doubly, triply or quadruply, identically or differently substituted phenyl,
where the substituents are selected from cyano, halogen, nitro, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₁-C₄)alkoxy, (C₁-C₄)haloalkoxy, (C₃-C₆)cycloalkyl, (C₃-C₆)halocycloalkyl, (C₁-C₄)alkyl-(C₃-C₆)cycloalkyl, (C₁-C₄)haloalkyl-(C₃-C₆)cycloalkyl, (C₁-C₄)haloalkenoxy, (C₁-C₄)alkylthio, (C₁-C₄)haloalkylthio, (C₃-C₆)cycloalkylthio, (C₃-C₆)halocycloalkylthio, (C₁-C₄)alkylsulphinyl, (C₁-C₄)haloalkylsulphinyl, (C₁-C₄)alkylsulphonyl, (C₁-C₄)haloalkylsulphonyl, (C₁-C₄)haloalkylsulphanyl, (C₃-C₆)cycloalkylsulphanyl, (C₁-C₄)alkylcarbonyl, (C₁-C₄)haloalkylcarbonyl, di-(C₁-C₄)alkylamino, di-(C₁-C₄)haloalkylamino.

In a particularly preferred embodiment, the substituents are selected as in Configuration (5-1) or (5-2), especially as described in Configuration (5-2).

In a further preferred embodiment, the invention relates to the compounds of the general formula (I-c)

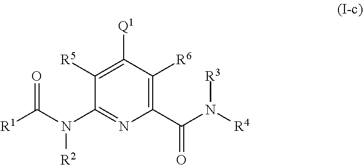

where R¹, R², R³, R⁴, R⁵, R⁶ and Q¹ have the definitions described above, especially the definitions described in Configuration (1-1) or Configuration (2-1) or Configuration (3-1) or Configuration (4-1) or Configuration (5-1) or Configuration (6-1) or the definitions described in Configuration (1-2) or Configuration (2-2) or Configuration (3-2) or Configuration (4-2) or Configuration (5-2) or Configuration (6-2).

In a further embodiment, the invention relates to the compounds of the formula (I) in which R³ and R⁴ together are not alkyl or alkenyl, such that no ring is formed, where R¹, R², R⁵, R⁶, Q¹, V¹ and V² have the definitions described above, especially the definitions described in Configuration (1-1) or Configuration (2-1) or Configuration (3-1) or Configuration (4-1) or Configuration (5-1) or Configuration (6-1) or the definitions described in Configuration (1-2) or Configuration (2-2) or Configuration (3-2) or Configuration (4-2) or Configuration (5-2) or Configuration (6-2).

In a further preferred embodiment, the invention relates to the compounds of the formula (I) in which R¹ is (C₁-C₄)alkyl, (C₁-C₄)haloalkyl, (C₃-C₆)cycloalkyl or (C₃-C₆)cycloalkyl-(C₁-C₄)alkyl, more preferably (C₁-C₄)alkyl or (C₃-C₆)cycloalkyl, most preferably (C₃-C₆)cycloalkyl, where R², R³, R⁴, R⁵, R⁶, Q¹, V¹ and V² have the definitions described above, especially the definitions described in Configuration (1-1) or Configuration (2-1) or Configuration (3-1) or Configuration (4-1) or Configuration (5-1) or Configuration (6-1) or the definitions described in Configuration (1-2) or Configuration (2-2) or Configuration (3-2) or Configuration (4-2) or Configuration (5-2) or Configuration (6-2).

In a further embodiment, the invention relates to the compounds of the formula (I) in which R⁴ is mono-phenyl-substituted methyl, where phenyl may optionally be mono-, di- or trisubstituted identically or differently by cyano, fluorine, chlorine, nitro, methyl, trifluoromethyl, methoxy, methyloxycarbonyl, thiocarbamoyl, aminosulphonyl, methylpyrazolyl, oxazolyl or oxadiazolyl, where R¹, R², R³, R⁵, R⁶, Q¹, V¹ and V² have the definitions described above, especially the definitions described in Configuration (1-1) or Configuration (2-1) or Configuration (3-1) or Configuration (4-1) or Configuration (5-1) or Configuration (6-1) or the definitions described in Configuration (1-2) or Configuration (2-2) or Configuration (3-2) or Configuration (4-2) or Configuration (5-2) or Configuration (6-2).

Likewise very particularly preferred compounds according to the invention are the compounds of the general formula (I) shown in Table 1.

Optionally substituted radicals may be mono- or polysubstituted, where the substituents in the case of polysubstitution may be the same or different.

The radical definitions or elucidations given in general terms or listed within ranges of preference apply correspondingly to end products and to starting materials and intermediates. These radical definitions can be combined with one another as desired, i.e. including combinations between the respective areas of preference.

Preference is given in accordance with the invention to compounds of the formula (I) in which there is a combination of the definitions listed above as being preferred.

Particular preference is given in accordance with the invention to compounds of the formula (I) in which there is a combination of the definitions listed above as being particularly preferred.

Very particular preference is given in accordance with the invention to compounds of the formula (I) in which there is a combination of the definitions listed above as being very particularly preferred.

Very especial preference is given in accordance with the invention to compounds of the formula (I) in which there is a combination of the definitions listed above as being very especially preferred.

Most preference is given in accordance with the invention to compounds of the formula (I) in which there is a combination of the definitions listed above as being most preferred.

The compounds of the formula (I) may possibly also, depending on the nature of the substituents, be in the form of stereoisomers, i.e. in the form of geometric and/or optical isomers or isomer mixtures of varying composition. These stereoisomers are, for example, enantiomers, diastereomers, atropisomers or geometric isomers. This invention provides both the pure stereoisomers and any desired mixtures of these isomers, even though it is generally only compounds of the formula (I) that are discussed here.

However, preference is given in accordance with the invention to using the optically active, stereoisomeric forms of the compounds of the formula (I) and salts thereof.

The invention therefore relates both to the pure enantiomers and diastereomers and to mixtures thereof for controlling animal pests, including arthropods and particularly insects.

If appropriate, the compounds of the formula (I) may be present in various polymorphic forms or as a mixture of various polymorphic forms. Both the pure polymorphs and the polymorph mixtures are provided by the invention and can be used in accordance with the invention.

The present compounds of the general formula (I) may optionally have a chiral carbon atom.

According to the rules of Cahn, Ingold and Prelog (CIP rules), these substituents may have either an (R) configuration or an (S) configuration.

The present invention encompasses compounds of the general formula (I) both with (S) and with (R) configuration at the particular chiral carbon atoms, i.e. the present invention encompasses the compounds of the general formula (I) in which the carbon atoms in question each independently have
(1) an (R) configuration; or
(2) an (S) configuration.

When more than one chiral centre is present in the compounds of the general formula (I), any desired combinations of the configurations of the chiral centres are possible, which means that
(1) one chiral centre may have the (R) configuration and the other chiral centre the (S) configuration;
(2) one chiral centre may have the (R) configuration and the other chiral centre the (R) configuration; and
(3) one chiral centre may have the (S) configuration and the other chiral centre the (S) configuration.

In the definitions listed in general or within areas of preference, unless stated otherwise, halogen is selected from the group of fluorine, chlorine, bromine and iodine, preferably in turn from the group of fluorine, chlorine and bromine.

In the context of the present invention, unless defined differently elsewhere, the term “alkyl”, either on its own or else in combination with further terms, for example haloalkyl, is understood to mean a radical of a saturated aliphatic hydrocarbyl group which has 1 to 12 carbon atoms and may be branched or unbranched. Examples of C₁-C₁₂-alkyl radicals are methyl, ethyl, n-propyl, isopropyl, n-butyl, isobutyl, sec-butyl, tert-butyl, n-pentyl, isopentyl, neopentyl, tert-pentyl, 1-methylbutyl, 2-methylbutyl, 1-ethylpropyl, 1,2-dimethylpropyl, hexyl, n-heptyl, n-octyl, n-nonyl, n-decyl, n-undecyl and n-dodecyl. Among these alkyl radicals, particular preference is given to C₁-C₆-alkyl radicals. Special preference is given to C₁-C₄-alkyl radicals.

According to the invention, unless defined differently elsewhere, the term “alkenyl”, either on its own or else in combination with further terms, is understood to mean a straight-chain or branched C₂-C₁₂-alkenyl radical which has at least one double bond, for example vinyl, allyl, 1-propenyl, isopropenyl, 1-butenyl, 2-butenyl, 3-butenyl, 1,3-butadienyl, 1-pentenyl, 2-pentenyl, 3-pentenyl, 4-pentenyl, 1,3-pentadienyl, 1-hexenyl, 2-hexenyl, 3-hexenyl, 4-hexenyl, 5-hexenyl and 1,4-hexadienyl. Among these, preference is given to C₂-C₆-alkenyl radicals and particular preference to C₂-C₄-alkenyl radicals.

According to the invention, unless defined differently elsewhere, the term “alkynyl”, either on its own or else in combination with further terms, is understood to mean a straight-chain or branched C₂-C₁₂-alkynyl radical which has at least one triple bond, for example ethynyl, 1-propynyl and propargyl. Among these, preference is given to C₃-C₆-alkynyl radicals and particular preference to C₃-C₄-alkynyl radicals. The alkynyl radical may also contain at least one double bond.

According to the invention, unless defined differently elsewhere, the term “cycloalkyl”, either on its own or else in combination with further terms, is understood to mean a C₃-C₈-cycloalkyl radical, for example cyclopropyl, cyclobutyl, cyclopentyl, cyclohexyl, cycloheptyl and cyclooctyl. Among these, preference is given to C₃-C₆-cycloalkyl radicals.

The term “alkoxy” or “alkyloxy”, either on its own or else in combination with further terms, for example haloalkoxy, is understood in the present case to mean an O-alkyl radical, where the term “alkyl” is as defined above.

Unless defined differently elsewhere, “oxy” means the —O— group.

Halogen-substituted radicals, for example haloalkyl, are mono- or polyhalogenated, up to the maximum number of possible substituents. In the case of polyhalogenation, the halogen atoms may be identical or different. In this case, halogen represents fluorine, chlorine, bromine or iodine, in particular fluorine, chlorine or bromine.

Unless defined differently elsewhere, the term “aryl” is understood in accordance with the invention to mean an aromatic radical having 6 to 14 carbon atoms, preferably phenyl.

Unless defined differently elsewhere, the term “arylalkyl” is understood to mean a combination of the radicals “aryl” and “alkyl” defined in accordance with the invention, the radical generally being bonded via the alkyl group. Examples of these are benzyl, phenylethyl or α-methylbenzyl, particular preference being given to benzyl.

Unless defined differently elsewhere, “hetaryl” denotes a mono-, bi- or tricyclic heterocyclic group composed of carbon atoms and at least one heteroatom, where at least one cycle is aromatic.

Preferably, the hetaryl group contains 3, 4, 5 or 6 carbon atoms. Hetaryl is preferably pyridinyl or pyrimidinyl.

Unless defined differently elsewhere, “heterocyclyl” denotes a monocyclic, saturated or partially saturated 4-, 5-, 6- or 7-membered ring of carbon atoms and at least one heteroatom in the ring. Preferably, the heterocyclyl group contains 3, 4, 5 or 6 carbon atoms and 1 or 2 heteroatoms from the group consisting of oxygen, sulphur and nitrogen. Examples ofheterocyclyl are azetidinyl, azolidinyl, azinanyl, oxetanyl, oxolanyl, oxanyl, dioxanyl, thietanyl, thiolanyl, thianyl and tetrahydrofuryl.

Unless defined differently elsewhere, “oxohetaryl” is a hetaryl containing, at at least one position in the ring, a ring carbon atom substituted by an (═O) group, which then results in the —C(═O)— group, where the carbon atom is part of the ring.

Unless defined differently elsewhere, “oxoheterocycyl” is a heterocyclyl containing, at at least one position in the ring, a ring carbon atom substituted by an (═O) group, which then results in the —C(═O)— group, where the carbon atom is part of the ring. Preferably, oxoheterocyclyl contains one or two —C(═O)— groups. Examples are oxotetrahydrofuranyl or pyrrolidinonyl.

The “benzylimino” or “benzylimine” group can also be referred to as phenylmethyleneamino. For example, the 4-cyanobenzylimine (R⁴ in Example No. 1-252) or 3-fluoro-4-(trifluoromethyl)benzylimine (R⁴ in Example No. 1-257) groups can also be referred to as (4-cyanophenyl)methyleneamino and [3-fluoro-4-(trifluoromethyl)phenyl]methyleneamino respectively. A crucial factor for the meaning of the term “benzylimino” or “benzylimine” in each case is the underlying chemical structure, i.e.

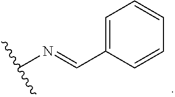 [SMILES: */N=C/c1ccccc1]

### Isomers

Depending on the nature of the substituents, the compounds of the formula (I) may be in the form of geometric and/or optically active isomers or corresponding isomer mixtures in different compositions. These stereoisomers are, for example, enantiomers, diastereomers, atropisomers or geometric isomers. The invention therefore encompasses both pure stereoisomers and any desired mixtures of these isomers.

### Methods and Uses

The invention also relates to methods for controlling animal pests, in which compounds of the formula (I) are allowed to act on animal pests and/or their habitat. The control of the animal pests is preferably conducted in agriculture and forestry, and in material protection. This preferably excludes methods for surgical or therapeutic treatment of the human or animal body and diagnostic methods carried out on the human or animal body.

The invention further relates to the use of the compounds of the formula (I) as pesticides, especially crop protection compositions.

In the context of the present application, the term “pesticide” in each case also always encompasses the term “crop protection composition”.

The compounds of the formula (I), given good plant tolerance, favourable endotherm toxicity and good environmental compatibility, are suitable for protecting plants and plant organs against biotic and abiotic stress factors, for increasing harvest yields, for improving the quality of the harvested material and for controlling animal pests, especially insects, arachnids, helminths, especially nematodes and molluscs, which are encountered in agriculture, in horticulture, in animal husbandry, in aquatic cultures, in forests, in gardens and leisure facilities, in the protection of stored products and of materials, and in the hygiene sector.

In the context of the present patent application, the term “hygiene” should be understood to mean any and all measures, provisions and procedures which have the aim of preventing diseases, especially infection diseases, and which serve to protect the health of humans and animals and/or protect the environment and/or maintain cleanliness. According to the invention, this especially includes measures for cleaning, disinfection and sterilization, for example of textiles or hard surfaces, especially surfaces made of glass, wood, cement, porcelain, ceramic, plastic or else metal(s), in order to ensure that these are free of hygiene pests and/or their secretions. The scope of protection of the invention in this regard preferably excludes surgical or therapeutic treatment procedures to be applied to the human body or the bodies of animals, and diagnostic procedures which are conducted on the human body or the bodies of animals.

The term “hygiene sector” covers all areas, technical fields and industrial applications in which these hygiene measures, provisions and procedures are important, for example with regard to hygiene in kitchens, bakeries, airports, bathrooms, swimming pools, department stores, hotels, hospitals, stables, animal keeping, etc.

The term “hygiene pest” should therefore be understood to mean one or more animal pests whose presence in the hygiene sector is problematic, especially for reasons of health. A main aim is therefore that of avoiding, or limiting to a minimum degree, the presence of hygiene pests and/or the exposure to these in the hygiene sector. This can especially be achieved through the use of a pesticide which can be used both for prevention of infestation and for prevention of an existing infestation. It is also possible to use formulations which prevent or reduce exposure to pests. Hygiene pests include, for example, the organisms mentioned below.

The term “hygiene protection” thus covers all acts by which these hygiene measures, provisions and procedures are maintained and/or improved.

The compounds of the formula (I) can preferably be used as pesticides. They are active against normally sensitive and resistant species and also against all or specific stages of development. The abovementioned pests include:

pests from the phylum of the Arthropoda, especially from the class of the Arachnida, for example Acarus spp., e.g. Acarus siro, Aceria kuko, Aceria sheldoni, Aculops spp., Aculus spp., e.g. Aculus fockeui, Aculus schlechtendali, Amblyomma spp., Amphitetranychus viennensis, Argas spp., Boophilus spp., Brevipalpus spp., e.g. Brevipalpus phoenicis, Bryobia graminum, Bryobia praetiosa, Centruroides spp., Chorioptes spp., Dermanyssus gallinae, Dermatophagoides pteronyssinus, Dermatophagoides farinae, Dermacentor spp., Eotetranychus spp., e.g. Eotetranychus hicoriae, Epitrimerus pyri, Eutetranychus spp., e.g. Eutetranychus banksi, Eriophyes spp., e.g. Eriophyes pyri, Glycyphagus domesticus, Halotydeus destructor, Hemitarsonemus spp., e.g. Hemitarsonemus latus (=Polyphagotarsonemus latus), Hyalomma spp., Ixodes spp., Latrodectus spp., Loxosceles spp., Neutrombicula autumnalis, Nuphersa spp., Oligonychus spp., e.g. Oligonychus coffeae, Oligonychus coniferarum, Oligonychus ilicis, Oligonychus indicus, Oligonychus mangiferus, Oligonychus pratensis, Oligonychus punicae, Oligonychus yothersi, Omithodorus spp., Omithonyssus spp., Panonychus spp., e.g. Panonychus citri (=Metatetranychus citri), Panonychus ulmi (=Metatetranychus ulmi), Phyllocoptruta oleivora, Platytetranychus multidigituli, Polyphagotarsonemus latus, Psoroptes spp., Rhipicephalus spp., Rhizoglyphus spp., Sarcoptes spp., Scorpio maurus, Steneotarsonemus spp., Steneotarsonemus spinki, Tarsonemus spp., e.g. Tarsonemus confusus, Tarsonemus pallidus, Tetranychus spp., e.g. Tetranychus canadensis, Tetranychus cinnabarinus, Tetranychus turkestani, Tetranychus urticae, Trombicula alfreddugesi, Vaejovis spp., Vasates lycopersici;
from the class of the Chilopoda, for example Geophilus spp., Scutigera spp.;
from the order or the class of the Collembola, for example Onychiurus armatus; Sminthurus viridis;
from the class of the Diplopoda, for example Blaniulus guttulatus;
from the class of the Insecta, for example from the order of the Blattodea, e.g. Blatta orientalis, Blattella asahinai, Blattella germanica, Leucophaea maderae, Loboptera decipiens, Neostylopyga rhombifolia, Panchlora spp., Parcoblatta spp., Periplaneta spp., e.g. Periplaneta americana, Periplaneta australasiae, Pycnoscelus surinamensis, Supella longipalpa;
from the order of the Coleoptera for example Acalymma vittatum, Acanthoscelides obtectus, Adoretus spp., Aethina tumida, Agelastica alni, Agrilus spp., e.g. Agrilus planipennis, Agrilus coxalis, Agrilus bilineatus, Agrilus anxius, Agriotes spp., e.g. Agriotes linneatus, Agriotes mancus, Alphitobius diaperinus, Amphimallon solstitialis, Anobium punctatum, Anoplophora spp., e.g. Anoplophora glabripennis, Anthonomus spp., e.g. Anthonomus grandis, Anthrenus spp., Apion spp., Apogonia spp., Atomaria spp., e.g. Atomaria linearis, Attagenus spp., Baris caerulescens, Bruchidius obtectus, Bruchus spp., e.g. Bruchus pisorum, Bruchus rufimanus, Cassida spp., Cerotoma trifurcata, Ceutorrhynchus spp., e.g. Ceutorrhynchus assimilis, Ceutorrhynchus quadridens, Ceutorrhynchus rapae, Chaetocnema spp., e.g. Chaetocnema confinis, Chaetocnema denticulata, Chaetocnema ectypa, Cleonus mendicus, Conoderus spp., Cosmopolites spp., e.g. Cosmopolites sordidus, Costelytra zealandica, Ctenicera spp., Curculio spp., e.g. Curculio caryae, Curculio caryatrypes, Curculio obtusus, Curculio sayi, Cryptolestes ferrugineus, Cryptolestes pusillus, Cryptorhynchus lapathi, Cryptorhynchus mangiferae, Cylindrocopturus spp., Cylindrocopturus adspersus, Cylindrocopturus fumissi, Dendroctonus spp., e.g. Dendroctonus ponderosae, Dermestes spp., Diabrotica spp., e.g. Diabrotica balteata, Diabrotica barberi, Diabrotica undecimpunctata howardi, Diabrotica undecimpunctata undecimpunctata, Diabrotica virgifera virgifera, Diabrotica virgifera zeae, Dichocrocis spp., Dicladispa armigera, Diloboderus spp., Epicaerus spp., Epilachna spp., e.g. Epilachna borealis, Epilachna varivestis, Epitrix spp., e.g. Epitrix cucumeris, Epitrix fuscula, Epitrix hirtipennis, Epitrix subcrinita, Epitrix tuberis, Faustinus spp., Gibbium psylloides, Gnathocerus comutus, Hellula undalis, Heteronychus arator, Heteronyx spp., Hylamorpha elegans, Hylotrupes bajulus, Hypera postica, Hypomeces squamosus, Hypothenemus spp., e.g. Hypothenemus hampei, Hypothenemus obscurus, Hypothenemus pubescens, Lachnostema consanguinea, Lasioderma serricome, Latheticus oryzae, Lathridius spp., Lema spp., Leptinotarsa decemlineata, Leucoptera spp., e.g. Leucoptera coffeella, Lissorhoptrus oryzophilus, Listronotus (=Hyperodes) spp., Lixus spp., Luperodes spp., Luperomorpha xanthodera, Lyctus spp., Megacyllene spp., e.g. Megacyllene robiniae, Megascelis spp., Melanotus spp., e.g. Melanotus longulus oregonensis, Meligethes aeneus, Melolontha spp., e.g. Melolontha melolontha, Migdolus spp., Monochamus spp., Naupactus xanthographus, Necrobia spp., Neogalerucella spp., Niptus hololeucus, Oryctes rhinoceros, Oryzaephilus surinamensis, Oryzaphagus oryzae, Otiorhynchus spp., e.g. Otiorhynchus cribricollis, Otiorhynchus ligustici, Otiorhynchus ovatus, Otiorhynchus rugosostriarus, Otiorhynchus sulcatus, Oulema spp., e.g. Oulema melanopus, Oulema oryzae, Oxycetonia jucunda, Phaedon cochleariae, Phyllophaga spp., Phyllophaga helleri, Phyllotreta spp., e.g. Phyllotreta armoraciae, Phyllotreta pusilla, Phyllotreta ramosa, Phyllotreta striolata, Popillia japonica, Premnotrypes spp., Prostephanus truncatus, Psylliodes spp., e.g. Psylliodes affinis, Psylliodes chrysocephala, Psylliodes punctulata, Ptinus spp., Rhizobius ventralis, Rhizopertha dominica, Rhynchophorus spp., Rhynchophorus ferrugineus, Rhynchophorus palmarum, Scolytus spp., e.g. Scolytus multistriatus, Sinoxylon perforans, Sitophilus spp., e.g. Sitophilus granarius, Sitophilus linearis, Sitophilus oryzae, Sitophilus zeamais, Sphenophorus spp., Stegobium paniceum, Stemechus spp., e.g. Stemechus paludatus, Symphyletes spp., Tanymecus spp., e.g. Tanymecus dilaticollis, Tanymecus indicus, Tanymecus palliatus, Tenebrio molitor, Tenebrioides mauretanicus, Tribolium spp., e.g. Tribolium audax, Tribolium castaneum, Tribolium confusum, Trogoderma spp., Tychius spp., Xylotrechus spp., Zabrus spp., e.g. Zabrus tenebrioides;
from the order of the Dermaptera, for example Anisolabis maritime, Forficula auricularia, Labidura riparia;
from the order of the Diptera, for example Aedes spp., e.g. Aedes aegypti, Aedes albopictus, Aedes sticticus, Aedes vexans, Agromyza spp., e.g. Agromyza frontella, Agromyza parvicomis, Anastrepha spp., Anopheles spp., e.g. Anopheles quadrimaculatus, Anopheles gambiae, Asphondylia spp., Bactrocera spp., e.g. Bactrocera cucurbitae, Bactrocera dorsalis, Bactrocera oleae, Bibio hortulanus, Calliphora erythrocephala, Calliphora vicina, Ceratitis capitata, Chironomus spp., Chrysomya spp., Chrysops spp., Chrysozona pluvialis, Cochliomya spp., Contarinia spp., e.g. Contarinia johnsoni, Contarinia nasturtii, Contarinia pyrivora, Contarinia schulzi, Contarinia sorghicola, Contarinia tritici, Cordylobia anthropophaga, Cricotopus sylvestris, Culex spp., e.g. Culex pipiens, Culex quinquefasciatus, Culicoides spp., Culiseta spp., Cuterebra spp., Dacus oleae, Dasineura spp., e.g. Dasineura brassicae, Delia spp., e.g. Delia antiqua, Delia coarctata, Delia florilega, Delia platura, Delia radicum, Dermatobia hominis, Drosophila spp., e.g. Drosphila melanogaster, Drosophila suzukii, Echinocnemus spp., Euleia heraclei, Fannia spp., Gasterophilus spp., Glossina spp., Haematopota spp., Hydrellia spp., Hydrellia griseola, Hylemya spp., Hippobosca spp., Hypoderma spp., Liriomyza spp., e.g. Liriomyza brassicae, Liriomyza huidobrensis, Liriomyza sativae, Lucilia spp., e.g. Lucilia cuprina, Lutzomyia spp., Mansonia spp., Musca spp., e.g. Musca domestica, Musca domestica vicina, Oestrus spp., Oscinella frit, Paratanytarsus spp., Paralauterbomiella subcincta, Pegomya oder Pegomyia spp., e.g. Pegomya betae, Pegomya hyoscyami, Pegomya rubivora, Phlebotomus spp., Phorbia spp., Phormia spp., Piophila casei, Platyparea poeciloptera, Prodiplosis spp., Psila rosae, Rhagoletis spp., e.g. Rhagoletis cingulata, Rhagoletis completa, Rhagoletis fausta, Rhagoletis indifferens, Rhagoletis mendax, Rhagoletis pomonella, Sarcophaga spp., Simulium spp., e.g. Simulium meridionale, Stomoxys spp., Tabanus spp., Tetanops spp., Tipula spp., e.g. Tipula paludosa, Tipula simplex, Toxotrypana curvicauda;
from the order of the Hemiptera, for example Acizzia acaciaebaileyanae, Acizzia dodonaeae, Acizzia uncatoides, Acrida turrita, Acyrthosipon spp., e.g. Acyrthosiphon pisum, Acrogonia spp., Aeneolamia spp., Agonoscena spp., Aleurocanthus spp., Aleyrodes proletella, Aleurolobus barodensis, Aleurothrixus floccosus, Allocaridara malayensis, Amrasca spp., e.g. Amrasca bigutulla, Amrasca devastans, Anuraphis cardui, Aonidiella spp., e.g. Aonidiella aurantii, Aonidiella citrina, Aonidiella inomata, Aphanostigma piri, Aphis spp., e.g. Aphis citricola, Aphis craccivora, Aphis fabae, Aphis forbesi, Aphis glycines, Aphis gossypii, Aphis hederae, Aphis illinoisensis, Aphis middletoni, Aphis nasturtii, Aphis nerii, Aphis pomi, Aphis spiraecola, Aphis vibumiphila, Arboridia apicalis, Arytainilla spp., Aspidiella spp., Aspidiotus spp., e.g. Aspidiotus nerii, Atanus spp., Aulacorthum solani, Bemisia tabaci, Blastopsylla occidentalis, Boreioglycaspis melaleucae, Brachycaudus helichrysi, Brachycolus spp., Brevicoryne brassicae, Cacopsylla spp., e.g. Cacopsylla pyricola, Calligypona marginata, Capulinia spp., Cameocephala fulgida, Ceratovacuna lanigera, Cercopidae, Ceroplastes spp., Chaetosiphon fragaefolii, Chionaspis tegalensis, Chlorita onukii, Chondracris rosea, Chromaphis juglandicola, Chrysomphalus aonidum, Chrysomphalus ficus, Cicadulina mbila, Coccomytilus halli, Coccus spp., e.g. Coccus hesperidum, Coccus longulus, Coccus pseudomagnoliarum, Coccus viridis, Cryptomyzus ribis, Cryptoneossa spp., Ctenarytaina spp., Dalbulus spp., Dialeurodes chittendeni, Dialeurodes citri, Diaphorina citri, Diaspis spp., Diuraphis spp., Doralis spp., Drosicha spp., Dysaphis spp., e.g. Dysaphis apiifolia, Dysaphis plantaginea, Dysaphis tulipae, Dysmicoccus spp., Empoasca spp., e.g. Empoasca abrupta, Empoasca fabae, Empoasca maligna, Empoasca solana, Empoasca stevensi, Eriosoma spp., e.g. Eriosoma americanum, Eriosoma lanigerum, Eriosoma pyricola, Erythroneura spp., Eucalyptolyma spp., Euphyllura spp., Euscelis bilobatus, Ferrisia spp., Fiorinia spp., Furcaspis oceanica, Geococcus coffeae, Glycaspis spp., Heteropsylla cubana, Heteropsylla spinulosa, Homalodisca coagulata, Hyalopterus arundinis, Hyalopterus pruni, Icerya spp., e.g. Icerya purchasi, Idiocerus spp., Idioscopus spp., Laodelphax striatellus, Lecanium spp., e.g. Lecanium comi (=Parthenolecanium comi), Lepidosaphes spp., e.g. Lepidosaphes ulmi, Lipaphis erysimi, Lopholeucaspis japonica, Lycorma delicatula, Macrosiphum spp., e.g. Macrosiphum euphorbiae, Macrosiphum lilii, Macrosiphum rosae, Macrosteles facifrons, Mahanarva spp., Melanaphis sacchari, Metcalfiella spp., Metcalfa pruinosa, Metopolophium dirhodum, Monellia costalis, Monelliopsis pecanis, Myzus spp., e.g. Myzus ascalonicus, Myzus cerasi, Myzus ligustri, Myzus omatus, Myzus persicae, Myzus nicotianae, Nasonovia ribisnigri, Neomaskellia spp., Nephotettix spp., e.g. Nephotettix cincticeps, Nephotettix nigropictus, Nettigoniclla spectra, Nilaparvata lugens, Oncometopia spp., Orthezia praelonga, Oxya chinensis, Pachypsylla spp., Parabemisia myricae, Paratrioza spp., e.g. Paratrioza cockerelli, Parlatoria spp., Pemphigus spp., e.g. Pemphigus bursarius, Pemphigus populivenae, Peregrinus maidis, Perkinsiella spp., Phenacoccus spp., e.g. Phenacoccus madeirensis, Phloeomyzus passerinii, Phorodon humuli, Phylloxera spp., e.g. Phylloxera devastatrix, Phylloxera notabilis, Pinnaspis aspidistrae, Planococcus spp., e.g. Planococcus citri, Prosopidopsylla flava, Protopulvinaria pyriformis, Pseudaulacaspis pentagona, Pseudococcus spp., e.g. Pseudococcus calceolariae, Pseudococcus comstocki, Pseudococcus longispinus, Pseudococcus maritimus, Pseudococcus vibumi, Psyllopsis spp., Psylla spp., e.g. Psylla buxi, Psylla mali, Psylla pyri, Pteromalus spp., Pulvinaria spp., Pyrilla spp., Quadraspidiotus spp., e.g. Quadraspidiotus juglansregiae, Quadraspidiotus ostreaeformis, Quadraspidiotus perniciosus, Quesada gigas, Rastrococcus spp., Rhopalosiphum spp., e.g. Rhopalosiphum maidis, Rhopalosiphum oxyacanthae, Rhopalosiphum padi, Rhopalosiphum rufiabdominale, Saissetia spp., e.g. Saissetia coffeae, Saissetia miranda, Saissetia neglecta, Saissetia oleae, Scaphoideus titanus, Schizaphis graminum, Selenaspidus articulatus, Sipha flava, Sitobion avenae, Sogata spp., Sogatella furcifera, Sogatodes spp., Stictocephala festina, Siphoninus phillyreae, Tenalaphara malayensis, Tetragonocephela spp., Tinocallis caryaefoliae, Tomaspis spp., Toxoptera spp., e.g. Toxoptera aurantii, Toxoptera citricidus, Trialeurodes vaporariorum, Trioza spp., e.g. Trioza diospyri, Typhlocyba spp., Unaspis spp., Viteus vitifolii, Zygina spp.;
from the suborder of the Heteroptera, for example Aelia spp., Anasa tristis, Antestiopsis spp., Boisea spp., Blissus spp., Calocoris spp., Campylomma livida, Cavelerius spp., Cimex spp., e.g. Cimex adjunctus, Cimex hemipterus, Cimex lectularius, Cimex pilosellus, Collaria spp., Creontiades dilutus, Dasynus piperis, Dichelops furcatus, Diconocoris hewetti, Dysdercus spp., Euschistus spp., e.g. Euschistus heros, Euschistus servus, Euschistus tristigmus, Euschistus variolarius, Eurydema spp., Eurygaster spp., Halyomorpha halys, Heliopeltis spp., Horcias nobilellus, Leptocorisa spp., Leptocorisa varicomis, Leptoglossus occidentalis, Leptoglossus phyllopus, Lygocoris spp., e.g. Lygocoris pabulinus, Lygus spp., e.g. Lygus elisus, Lygus hesperus, Lygus lineolaris, Macropes excavatus, Megacopta cribraria, Miridae, Monalonion atratum, Nezara spp., e.g. Nezara viridula, Nysius spp., Oebalus spp., Pentomidae, Piesma quadrata, Piezodorus spp., e.g. Piezodorus guildinii, Psallus spp., Pseudacysta persea, Rhodnius spp., Sahlbergella singularis, Scaptocoris castanea, Scotinophora spp., Stephanitis nashi, Tibraca spp., Triatoma spp.;
from the order of the Hymenoptera, for example Acromyrmex spp., Athalia spp., e.g. Athalia rosae, Atta spp., Camponotus spp., Dolichovespula spp., Diprion spp., e.g. Diprion similis, Hoplocampa spp., e.g. Hoplocampa cookei, Hoplocampa testudinea, Lasius spp., Linepithema (Iridiomyrmex) humile, Monomorium pharaonis, Paratrechina spp., Paravespula spp., Plagiolepis spp., Sirex spp., e.g. Sirex noctilio, Solenopsis invicta, Tapinoma spp., Technomyrmex albipes, Urocerus spp., Vespa spp., e.g. Vespa crabro, Wasmannia auropunctata, Xeris spp.;
from the order of the Isopoda, for example Armadillidium vulgare, Oniscus asellus, Porcellio scaber;
from the order of the Isoptera, for example Coptotermes spp., e.g. Coptotermes formosanus, Comitermes cumulans, Cryptotermes spp., Incisitermes spp., Kalotermes spp., Microtermes obesi, Nasutitermes spp., Odontotermes spp., Porotermes spp., Reticulitermes spp., e.g. Reticulitermes flavipes, Reticulitermes hesperus;
from the order of the Lepidoptera, for example Achroia grisella, Acronicta major, Adoxophyes spp., e.g. Adoxophyes orana, Aedia leucomelas, Agrotis spp., e.g. Agrotis segetum, Agrotis ipsilon, Alabama spp., e.g. Alabama argillacea, Amyelois transitella, Anarsia spp., Anticarsia spp., e.g. Anticarsia gemmatalis, Argyroploce spp., Autographa spp., Barathra brassicae, Blastodacna atra, Borbo cinnara, Bucculatrix thurberiella, Bupalus piniarius, Busseola spp., Cacoecia spp., Caloptilia theivora, Capua reticulana, Carpocapsa pomonella, Carposina niponensis, Cheimatobia brumata, Chilo spp., e.g. Chilo plejadellus, Chilo suppressalis, Choreutis pariana, Choristoneura spp., Chrysodeixis chalcites, Clysia ambiguella, Cnaphalocerus spp., Cnaphalocrocis medinalis, Cnephasia spp., Conopomorpha spp., Conotrachelus spp., Copitarsia spp., Cydia spp., e.g. Cydia nigricana, Cydia pomonella, Dalaca noctuides, Diaphania spp., Diparopsis spp., Diatraea saccharalis, Dioryctria spp., e.g. Dioryctria zimmermani, Earias spp., Ecdytolopha aurantium, Elasmopalpus lignosellus, Eldana saccharina, Ephestia spp., e.g. Ephestia elutella, Ephestia kuehniella, Epinotia spp., Epiphyas postvittana, Erannis spp., Erschoviella musculana, Etiella spp., Eudocima spp., Eulia spp., Eupoecilia ambiguella, Euproctis spp., e.g. Euproctis chrysorrhoea, Euxoa spp., Feltia spp., Galleria mellonella, Gracillaria spp., Grapholitha spp., e.g. Grapholita molesta, Grapholita prunivora, Hedylepta spp., Helicoverpa spp., e.g. Helicoverpa armigera, Helicoverpa zea, Heliothis spp., e.g. Heliothis virescens Hofmannophila pseudospretella, Homoeosoma spp., Homona spp., Hyponomeuta padella, Kakivoria flavofasciata, Lampides spp., Laphygma spp., Laspeyresia molesta, Leucinodes orbonalis, Leucoptera spp., e.g. Leucoptera coffeella, Lithocolletis spp., e.g. Lithocolletis blancardella, Lithophane antennata, Lobesia spp., e.g. Lobesia botrana, Loxagrotis albicosta, Lymantria spp., e.g. Lymantria dispar, Lyonetia spp., e.g. Lyonetia clerkella, Malacosoma neustria, Maruca testulalis, Mamestra brassicae, Melanitis leda, Mocis spp., Monopis obviella, Mythimna separata, Nemapogon cloacellus, Nymphula spp., Oiketicus spp., Omphisa spp., Operophtera spp., Oria spp., Orthaga spp., Ostrinia spp., e.g. Ostrinia nubilalis, Panolis flammea, Pamara spp., Pectinophora spp., e.g. Pectinophora gossypiella, Perileucoptera spp., Phthorimaea spp., e.g. Phthorimaea operculella, Phyllocnistis citrella, Phyllonorycter spp., e.g. Phyllonorycter blancardella, Phyllonorycter crataegella, Pieris spp., e.g. Pieris rapae, Platynota stultana, Plodia interpunctella, Plusia spp., Plutella xylostella (=Plutella maculipennis), Podesia spp., e.g. Podesia syringae, Prays spp., Prodenia spp., Protoparce spp., Pseudaletia spp., e.g. Pseudaletia unipuncta, Pseudoplusia includens, Pyrausta nubilalis, Rachiplusia nu, Schoenobius spp., e.g. Schoenobius bipunctifer, Scirpophaga spp., e.g. Scirpophaga innotata, Scotia segetum, Sesamia spp., e.g. Sesamia inferens, Sparganothis spp., Spodoptera spp., e.g. Spodoptera eradiana, Spodoptera exigua, Spodoptera frugiperda, Spodoptera praefica, Stathmopoda spp., Stenoma spp., Stomopteryx subsecivella, Synanthedon spp., Tecia solanivora, Thaumetopoea spp., Thermesia gemmatalis, Tinea cloacella, Tinea pellionella, Tineola bisselliella, Tortrix spp., Trichophaga tapetzella, Trichoplusia spp., e.g. Trichoplusia ni, Tryporyza incertulas, Tuta absoluta, Virachola spp.;
from the order of the Orthoptera or Saltatoria, for example Acheta domesticus, Dichroplus spp., Gryllotalpa spp., e.g. Gryllotalpa gryllotalpa, Hieroglyphus spp., Locusta spp., e.g. Locusta migratoria, Melanoplus spp., e.g. Melanoplus devastator, Paratlanticus ussuriensis, Schistocerca gregaria;
from the order of the Phthiraptera, for example Damalinia spp., Haematopinus spp., Linognathus spp., Pediculus spp., Phylloxera vastatrix, Phthirus pubis, Trichodectes spp.;
from the order of the Psocoptera, for example Lepinotus spp., Liposcelis spp.;
from the order of the Siphonaptera, for example Ceratophyllus spp., Ctenocephalides spp., e.g. Ctenocephalides canis, Ctenocephalides felis, Pulex irritans, Tunga penetrans, Xenopsylla cheopis;
from the order of the Thysanoptera, for example Anaphothrips obscurus, Baliothrips biformis, Chaetanaphothrips leeuweni, Drepanothrips reuteri, Enneothrips flavens, Frankliniella spp., e.g. Frankliniella fusca, Frankliniella occidentalis, Frankliniella schultzei, Frankliniella tritici, Frankliniella vaccinii, Frankliniella williamsi, Haplothrips spp., Heliothrips spp., Hercinothrips femoralis, Kakothrips spp., Rhipiphorothrips cruentatus, Scirtothrips spp., Taeniothrips cardamomi, Thrips spp., e.g. Thrips palmi, Thrips tabaci;
from the order of the Zygentoma (=Thysanura), for example Ctenolepisma spp., Lepisma saccharina, Lepismodes inquilinus, Thermobia domestica;
from the class of the Symphyla, for example Scutigerella spp., e.g. Scutigerella immaculata; pests from the phylum of the Mollusca, for example from the class of the Bivalvia, e.g. Dreissena spp.;
and also from the class of the Gastropoda, for example Arion spp., e.g. Arion ater rufus, Biomphalaria spp., Bulinus spp., Deroceras spp., e.g. Deroceras laeve, Galba spp., Lymnaea spp., Oncomelania spp., Pomacea spp., Succinea spp.;
plant pests from the phylum of the Nematoda, i.e. plant-parasitic nematodes, in particular Aglenchus spp., e.g. Aglenchus agricola, Anguina spp., e.g. Anguina tritici, Aphelenchoides spp., e.g. Aphelenchoides arachidis, Aphelenchoides fragariae, Belonolaimus spp., e.g. Belonolaimus gracilis, Belonolaimus longicaudatus, Belonolaimus nortoni, Bursaphelenchus spp., e.g. Bursaphelenchus cocophilus, Bursaphelenchus eremus, Bursaphelenchus xylophilus, Cacopaurus spp., e.g. Cacopaurus pestis, Criconemella spp., e.g. Criconemella curvata, Criconemella onoensis, Criconemella ornata, Criconemella rusium, Criconemella xenoplax (=Mesocriconema xenoplax), Criconemoides spp., e.g. Criconemoides femiae, Criconemoides onoense, Criconemoides ornatum, Ditylenchus spp., e.g. Ditylenchus dipsaci, Dolichodorus spp., Globodera spp., e.g. Globodera pallida, Globodera rostochiensis, Helicotylenchus spp., e.g. Helicotylenchus dihystera, Hemicriconemoides spp., Hemicycliophora spp., Heterodera spp., e.g. Heterodera avenae, Heterodera glycines, Heterodera schachtii, Hirschmaniella spp., Hoplolaimus spp., Longidorus spp., e.g. Longidorus africanus, Meloidogyne spp., e.g. Meloidogyne chitwoodi, Meloidogyne fallax, Meloidogyne hapla, Meloidogyne incognita, Meloinema spp., Nacobbus spp., Neotylenchus spp., Paralongidorus spp., Paraphelenchus spp., Paratrichodorus spp., e.g. Paratrichodorus minor, Paratylenchus spp., Pratylenchus spp., e.g. Pratylenchus penetrans, Pseudohalenchus spp., Psilenchus spp., Punctodera spp., Quinisulcius spp., Radopholus spp., e.g. Radopholus citrophilus, Radopholus similis, Rotylenchulus spp., Rotylenchus spp., Scutellonema spp., Subanguina spp., Trichodorus spp., e.g. Trichodorus obtusus, Trichodorus primitivus, Tylenchorhynchus spp., e.g. Tylenchorhynchus annulatus, Tylenchulus spp., e.g. Tylenchulus semipenetrans, Xiphinema spp., e.g. Xiphinema index.

The compounds of the formula (I) can, as the case may be, at certain concentrations or application rates, also be used as herbicides, safeners, growth regulators or agents to improve plant properties, as microbicides or gametocides, for example as fungicides, antimycotics, bactericides, virucides (including agents against viroids) or as agents against MLO (mycoplasma-like organisms) and RLO (rickettsia-like organisms). They can, as the case may be, also be used as intermediates or precursors for the synthesis of other active ingredients.

### Formulations

The present invention further relates to formulations and use forms prepared therefrom as pesticides, for example drench, drip and spray liquors, comprising at least one compound of the formula (I). Optionally, the use forms comprise further pesticides and/or adjuvants which improve action, such as penetrants, e.g. vegetable oils, for example rapeseed oil, sunflower oil, mineral oils, for example paraffin oils, alkyl esters of vegetable fatty acids, for example rapeseed oil methyl ester or soya oil methyl ester, or alkanol alkoxylates and/or spreaders, for example alkylsiloxanes and/or salts, for example organic or inorganic ammonium or phosphonium salts, for example ammonium sulphate or diammonium hydrogenphosphate and/or retention promoters, for example dioctyl sulphosuccinate or hydroxypropylguar polymers and/or humectants, for example glycerol and/or fertilizers, for example ammonium-, potassium- or phosphorus-containing fertilizers.

Customary formulations are, for example, water-soluble liquids (SL), emulsion concentrates (EC), emulsions in water (EW), suspension concentrates (SC, SE, FS, OD), water-dispersible granules (WG), granules (GR) and capsule concentrates (CS); these and further possible formulation types are described, for example, by Crop Life International and in Pesticide Specifications, Manual on development and use of FAO and WHO specifications for pesticides, FAO Plant Production and Protection Papers—173, prepared by the FAO/WHO Joint Meeting on Pesticide Specifications, 2004, ISBN: 9251048576. The formulations, in addition to one or more compounds of the formula (I), optionally comprise further active agrochemical ingredients.

Preference is given to formulations or use forms comprising auxiliaries, for example extenders, solvents, spontaneity promoters, carriers, emulsifiers, dispersants, frost protection agents, biocides, thickeners and/or further auxiliaries, for example adjuvants. An adjuvant in this context is a component which enhances the biological effect of the formulation, without the component itself having any biological effect. Examples of adjuvants are agents which promote retention, spreading, attachment to the leaf surface or penetration.

These formulations are produced in a known manner, for example by mixing the compounds of the formula (I) with auxiliaries, for example extenders, solvents and/or solid carriers and/or other auxiliaries, for example surfactants. The formulations are produced either in suitable facilities or else before or during application.

The auxiliaries used may be substances suitable for imparting special properties, such as certain physical, technical and/or biological properties, to the formulation of the compounds of the formula (I), or to the use forms prepared from these formulations (for example ready-to-use pesticides such as spray liquors or seed-dressing products).

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the simple and substituted amines, amides, lactams (such as N-alkylpyrrolidones) and lactones, the sulphones and sulphoxides (such as dimethyl sulphoxide).

If the extender utilized is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

In principle, it is possible to use all suitable solvents. Examples of suitable solvents are aromatic hydrocarbons, for example xylene, toluene or alkylnaphthalenes, chlorinated aromatic or chlorinated aliphatic hydrocarbons, for example chlorobenzene, chloroethylene or methylene chloride, aliphatic hydrocarbons, for example cyclohexane, paraffins, petroleum fractions, mineral and vegetable oils, alcohols, for example methanol, ethanol, isopropanol, butanol or glycol and their ethers and esters, ketones, for example acetone, methyl ethyl ketone, methyl isobutyl ketone or cyclohexanone, strongly polar solvents, for example dimethyl sulphoxide, and water.

In principle, it is possible to use all suitable carriers. Useful carriers especially include, for example, ammonium salts and natural, finely ground rocks, such as kaolins, aluminas, talc, chalk, quartz, attapulgite, montmorillonite or diatomaceous earth, and synthetic, finely ground rocks, such as highly disperse silica, aluminium oxide and natural or synthetic silicates, resins, waxes and/or solid fertilizers. It is likewise possible to use mixtures of such carriers. Useful carriers for granules include: for example crushed and fractionated natural rocks such as calcite, marble, pumice, sepiolite, dolomite, and synthetic granules of inorganic and organic flours, and also granules of organic material such as sawdust, paper, coconut shells, maize cobs and tobacco stalks.

It is also possible to use liquefied gaseous extenders or solvents. Especially suitable extenders or carriers are those which are gaseous at standard temperature and under atmospheric pressure, for example aerosol propellants such as halogenated hydrocarbons, and also butane, propane, nitrogen and carbon dioxide.

Examples of emulsifiers and/or foam formers, dispersants or wetting agents having ionic or nonionic properties or mixtures of these surface-active substances are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyl taurates), phosphoric esters of polyethoxylated alcohols or phenols, fatty acid esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is advantageous if one of the compounds of the formula (I) and/or one of the inert carriers is insoluble in water and if the application takes place in water.

Further auxiliaries which may be present in the formulations and the use forms derived therefrom include dyes such as inorganic pigments, for example iron oxide, titanium oxide and Prussian Blue, and organic dyes such as alizarin dyes, azo dyes and metal phthalocyanine dyes, and nutrients and trace nutrients such as salts of iron, manganese, boron, copper, cobalt, molybdenum and zinc.

Additional components which may be present are stabilizers, such as cold stabilizers, preservatives, antioxidants, light stabilizers, or other agents which improve chemical and/or physical stability. Foam generators or antifoams may also be present.

In addition, the formulations and the use forms derived therefrom may also comprise, as additional auxiliaries, stickers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids. Further auxiliaries may be mineral and vegetable oils.

It is possible if appropriate for still further auxiliaries to be present in the formulations and the use forms derived therefrom. Examples of such additives are fragrances, protective colloids, binders, adhesives, thickeners, thixotropic agents, penetrants, retention promoters, stabilizers, sequestrants, complexing agents, humectants, spreaders. In general, the compounds of the formula (I) can be combined with any solid or liquid additive commonly used for formulation purposes.

Useful retention promoters include all those substances which reduce dynamic surface tension, for example dioctyl sulphosuccinate, or increase viscoelasticity, for example hydroxypropylguar polymers.

Useful penetrants in the present context are all those substances which are typically used to improve the penetration of agrochemically active ingredients into plants. Penetrants are defined in this context by their ability to penetrate from the (generally aqueous) application liquor and/or from the spray coating into the cuticle of the plant and hence to increase the mobility of the active ingredients in the cuticle. The method described in the literature (Baur et al., 1997, Pesticide Science 51, 131-152) can be used for determining this property. Examples include alcohol alkoxylates such as coconut fatty ethoxylate (10) or isotridecyl ethoxylate (12), fatty acid esters, for example rapeseed oil methyl ester or soya oil methyl ester, fatty amine alkoxylates, for example tallowamine ethoxylate (15), or ammonium and/or phosphonium salts, for example ammonium sulphate or diammonium hydrogenphosphate.

The formulations preferably comprise between 0.00000001% and 98% by weight of the compound of the formula (I), more preferably between 0.01% and 95% by weight of the compound of the formula (I), most preferably between 0.5% and 90% by weight of the compound of the formula (I), based on the weight of the formulation.

The content of the compound of the formula (I) in the use forms prepared from the formulations (in particular pesticides) may vary within wide ranges. The concentration of the compound of the formula (I) in the use forms may typically be between 0.00000001% and 95% by weight of the compound of the formula (I), preferably between 0.00001% and 1% by weight, based on the weight of the use form. Application is accomplished in a customary manner appropriate for the use forms.

### Mixtures

The compounds of the formula (I) can also be used in a mixture with one or more suitable fungicides, bactericides, acaricides, molluscicides, nematicides, insecticides, microbiological agents, beneficial organisms, herbicides, fertilizers, bird repellents, phytotonics, sterilants, safeners, semiochemicals and/or plant growth regulators, in order thus, for example, to broaden the spectrum of action, prolong the period of action, enhance the rate of action, prevent repellency or prevent evolution of resistance. In addition, active ingredient combinations of this kind can improve plant growth and/or tolerance to abiotic factors, for example high or low temperatures, to drought or to elevated water content or soil salinity. It is also possible to improve flowering and fruiting performance, optimize germination capacity and root development, facilitate harvesting and improve yields, influence maturation, improve the quality and/or the nutritional value of the harvested products, prolong storage life and/or improve the processability of the harvested products.

In addition, the compounds of the formula (I) may be present in a mixture with other active ingredients or semiochemicals such as attractants and/or bird repellents and/or plant activators and/or growth regulators and/or fertilizers. Likewise, the compounds of the formula (I) can be used to improve plant properties, for example growth, yield and quality of the harvested material.

In a particular embodiment according to the invention, the compounds of the formula (I) are present in formulations or in the use forms prepared from these formulations in a mixture with further compounds, preferably those as described below.

If one of the compounds mentioned below can occur in different tautomeric forms, these forms are also included even if not explicitly mentioned in each case. All the mixing components mentioned, as the case may be, may also form salts with suitable bases or acids if they are capable of doing so on the basis of their functional groups.

### Insecticides/Acaricides/Nematicides

The active ingredients specified here with their common names are known and are described for example in “The Pesticide Manual”, 16th ed., British Crop Protection Council 2012, or can be searched for on the Internet (e.g. http://www.alanwood.net/pesticides). The classification is based on the IRAC Mode of Action Classification Scheme applicable at the time of filing of this patent application.

(1) Acetylcholinesterase (AChE) inhibitors, for example carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos-ethyl, azinphos-methyl, cadusafos, chlorethoxyfos, chlorfenvinphos, chlormephos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl 0-(methoxyaminothiophosphoryl) salicylate, isoxathion, malathion, mecarbam, methamidophos, methidathion, mevinphos, monocrotophos, naled, omethoate, oxydemeton-methyl, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.

(2) GABA-gated chloride channel blockers, for example cyclodiene-organochlorines, e.g. chlordane and endosulfan or phenylpyrazoles (fiproles), e.g. ethiprole and fipronil.

(3) Sodium channel modulators, for example pyrethroids, e.g. acrinathrin, allethrin, d-cis-trans allethrin, d-trans allethrin, bifenthrin, bioallethrin, bioallethrin S-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomer], deltamethrin, empenthrin [(EZ)-(1R) isomer], esfenvalerate, etofenprox, fenpropathrin, fenvalerate, flucythrinate, flumethrin, tau-fluvalinate, halfenprox, imiprothrin, kadethrin, momfluorothrin, permethrin, phenothrin [(1R)-trans isomer], prallethrin, pyrethrins (pyrethrum), resmethrin, silafluofen, tefluthrin, tetramethrin, tetramethrin [(1R) isomer], tralomethrin and transfluthrin or DDT or methoxychlor.

(4) Nicotinic acetylcholine receptor (nAChR) competitive modulators, for example neonicotinoids, e.g. acetamiprid, clothianidin, dinotefuran, imidacloprid, nitenpyram, thiacloprid and thiamethoxam or nicotine or sulfoxaflor or flupyradifurone.

(5) Nicotinic acetylcholine receptor (nAChR) allosteric modulators, for example spinosyns, e.g. spinetoram and spinosad.

(6) Glutamate-gated chloride channel (GluCl) allosteric modulators, for example avermectins/milbemycins, e.g. abamectin, emamectin benzoate, lepimectin and milbemectin.

(7) Juvenile hormone mimetics, for example juvenile hormone analogues, e.g. hydroprene, kinoprene and methoprene or fenoxycarb or pyriproxyfen.

(8) Miscellaneous non-specific (multisite) inhibitors, for example alkyl halides, e.g. methyl bromide and other alkyl halides; or chloropicrin or sulphuryl fluoride or borax or tartar emetic or methyl isocyanate generator, e.g. diazomet and metam.

(9) Chordotonal organ modulators, e.g. pymetrozine or flonicamide.

(10) Mite growth inhibitors, for example clofentezine, hexythiazox and diflovidazin or etoxazole.

(11) Microbial disruptors of the insect midgut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis and B.t. plant proteins: Cry1Ab, Cry1Ac, Cry1Fa, Cry1A.105, Cry2Ab, VIP3A, mCry3A, Cry3Ab, Cry3Bb, Cry34Ab1/35Ab1.

(12) Inhibitors of mitochondrial ATP synthase, such as ATP disruptors, for example diafenthiuron or organotin compounds, e.g. azocyclotin, cyhexatin and fenbutatin oxide or propargite or tetradifon.

(13) Uncouplers of oxidative phosphorylation via disruption of the proton gradient, for example chlorfenapyr, DNOC and sulfluramid.

(14) Nicotinic acetylcholine receptor channel blockers, for example bensultap, cartap hydrochloride, thiocyclam, and thiosultap-sodium.

(15) Inhibitors of chitin biosynthesis, type 0, for example bistrifluron, chlorfluazuron, diflubenzuron, flucycloxuron, flufenoxuron, hexaflumuron, lufenuron, novaluron, noviflumuron, teflubenzuron and triflumuron.

(16) Inhibitors of chitin biosynthesis, type 1, for example buprofezin.

(17) Moulting disruptors (especially in the case of Diptera), for example cyromazine.

(18) Ecdysone receptor agonists, for example chromafenozide, halofenozide, methoxyfenozide and tebufenozide.

(19) Octopamine receptor agonists, for example amitraz.

(20) Mitochondrial complex III electron transport inhibitors, for example hydramethylnon or acequinocyl or fluacrypyrim.

(21) Mitochondrial complex I electron transport inhibitors, for example METI acaricides, e.g. fenazaquin, fenpyroximate, pyrimidifen, pyridaben, tebufenpyrad and tolfenpyrad or rotenone (Derris).

(22) Voltage-dependent sodium channel blockers, for example indoxacarb or metaflumizone.

(23) Inhibitors of acetyl CoA carboxylase, for example tetronic and tetramic acid derivatives, e.g. spirodiclofen, spiromesifen and spirotetramat.

(24) Mitochondrial complex IV electron transport inhibitors, for example phosphines, e.g. aluminium phosphide, calcium phosphide, phosphine and zinc phosphide, or cyanides, calcium cyanide, potassium cyanide and sodium cyanide.

(25) Mitochondrial complex II electron transport inhibitors, for example beta-keto nitrile derivatives, e.g. cyenopyrafen and cyflumetofen and carboxanilides, for example pyflubumide.

(28) Ryanodine receptor modulators, for example diamides, e.g. chlorantraniliprole, cyantraniliprole and flubendiamide,

further active ingredients, for example afidopyropen, afoxolaner, azadirachtin, benclothiaz, benzoximate, bifenazate, broflanilide, bromopropylate, chinomethionat, chloroprallethrin, cryolite, cyclaniliprole, cycloxaprid, cyhalodiamide, dicloromezotiaz, dicofol, epsilon metofluthrin, epsilon momfluthrin, flometoquin, fluazaindolizine, fluensulfone, flufenerim, flufenoxystrobin, flufiprole, fluhexafon, fluopyram, fluralaner, fluxametamide, fufenozide, guadipyr, heptafluthrin, imidaclothiz, iprodione, kappa bifenthrin, kappa tefluthrin, lotilaner, meperfluthrin, paichongding, pyridalyl, pyrifluquinazon, pyriminostrobin, spirobudiclofen, tetramethylfluthrin, tetraniliprole, tetrachlorantraniliprole, tioxazafen, thiofluoximate, triflumezopyrim and iodomethane; additionally preparations based on Bacillus firmus (1-1582, BioNeem, Votivo), and the following compounds: 1-{2-fluoro-4-methyl-5-[(2,2,2-trifluoroethyl)sulphinyl]phenyl}-3-(trifluoromethyl)-1H-1,2,4-triazole-5-amine (known from WO2006/043635) (CAS 885026-50-6), {1′-[(2E)-3-(4-chlorophenyl)prop-2-en-1-yl]-5-fluorospiro[indole-3,4′-piperidine]-1(2H)-yl}(2-chloropyridin-4-yl)methanone (known from WO2003/106457) (CAS 637360-23-7), 2-chloro-N-[2-{1-[(2E)-3-(4-chlorophenyl)prop-2-en-1-yl]piperidin-4-yl}-4-(trifluoromethyl)phenyl]isonicotinamide (known from WO2006/003494) (CAS 872999-66-1), 3-(4-chloro-2,6-dimethylphenyl)-4-hydroxy-8-methoxy-1,8-diazaspiro[4.5]dec-3-en-2-one (known from WO 2010052161) (CAS 1225292-17-0), 3-(4-chloro-2,6-dimethylphenyl)-8-methoxy-2-oxo-1,8-diazaspiro[4.5]dec-3-en-4-yl ethylcarbonate (known from EP 2647626) (CAS-1440516-42-6), 4-(but-2-yn-1-yloxy)-6-(3,5-dimethylpiperidin-1-yl)-5-fluoropyrimidine (known from WO2004/099160) (CAS 792914-58-0), PF1364 (known from JP2010/018586) (CAS Reg. No. 1204776-60-2), N-[(2E)-1-[(6-chloropyridin-3-yl)methyl]pyridin-2(1H)-ylidene]-2,2,2-trifluoroacetamide (known from WO2012/029672) (CAS 1363400-41-2), (3E)-3-[1-[(6-chloro-3-pyridyl)methyl]-2-pyridylidene]-1,1,1-trifluoropropan-2-one (known from WO2013/144213) (CAS 1461743-15-6), N-[3-(benzylcarbamoyl)-4-chlorophenyl]-1-methyl-3-(pentafluoroethyl)-4-(trifluoromethyl)-1H-pyrazole-5-carboxamide (known from WO2010/051926) (CAS 1226889-14-0), 5-bromo-4-chloro-N-[4-chloro-2-methyl-6-(methylcarbamoyl)phenyl]-2-(3-chloro-2-pyridyl)pyrazole-3-carboxamide (known from CN103232431) (CAS 1449220-44-3), 4-[5-(3,5-dichlorophenyl)-4,5-dihydro-5-(trifluoromethyl)-3-isoxazolyl]-2-methyl-N-(cis-1-oxido-3-thietanyl)benzamide, 4-[5-(3,5-dichlorophenyl)-4,5-dihydro-5-(trifluoromethyl)-3-isoxazolyl]-2-methyl-N-(trans-1-oxido-3-thietanyl)benzamide and 4-[(5 S)-5-(3,5-dichlorophenyl)-4,5-dihydro-5-(trifluoromethyl)-3-isoxazolyl]-2-methyl-N-(cis-1-oxido-3-thietanyl)benzamide (known from WO 2013/050317 A1) (CAS 1332628-83-7), N-[3-chloro-1-(3-pyridinyl)-1H-pyrazol-4-yl]-N-ethyl-3-[(3,3,3-trifluoropropyl)sulphinyl]propanamide, (+)-N-[3-chloro-1-(3-pyridinyl)-1H-pyrazol-4-yl]-N-ethyl-3-[(3,3,3-trifluoropropyl)sulphinyl]propanamide and (−)-N-[3-chloro-1-(3-pyridinyl)-1H-pyrazol-4-yl]-N-ethyl-3-[(3,3,3-trifluoropropyl)sulphinyl]propanamide (known from WO 2013/162715 A2, WO 2013/162716 A2, US 2014/0213448 A1) (CAS 1477923-37-7), 5-[[(2E)-3-chloro-2-propen-1-yl]amino]-1-[2,6-dichloro-4-(trifluoromethyl)phenyl]-4-[(trifluoromethyl)sulphinyl]-1H-pyrazole-3-carbonitrile (known from CN 101337937 A) (CAS 1105672-77-2), 3-bromo-N-[4-chloro-2-methyl-6-[(methylamino)thioxomethyl]phenyl]-1-(3-chloro-2-pyridinyl)-1H-pyrazole-5-carboxamide, (Liudaibenjiaxuanan, known from CN 103109816 A) (CAS 1232543-85-9); N-[4-chloro-2-[[(1,1-dimethylethyl)amino]carbonyl]-6-methylphenyl]-1-(3-chloro-2-pyridinyl)-3-(fluoromethoxy)-1H-pyrazole-5-carboxamide (known from WO 2012/034403 A1) (CAS 1268277-22-0), N-[2-(5-amino-1,3,4-thiadiazol-2-yl)-4-chloro-6-methylphenyl]-3-bromo-1-(3-chloro-2-pyridinyl)-1H-pyrazole-5-carboxamide (known from WO 2011/085575 A1) (CAS 1233882-22-8), 4-[3-[2,6-dichloro-4-[(3,3-dichloro-2-propen-1-yl)oxy]phenoxy]propoxy]-2-methoxy-6-(trifluoromethyl)pyrimidine (known from CN 101337940 A) (CAS 1108184-52-6); (2E)- and 2(Z)-2-[2-(4-cyanophenyl)-1-[3-(trifluoromethyl)phenyl]ethylidene]-N-[4-(difluoromethoxy)phenyl]hydrazinecarboxamide (known from CN 101715774 A) (CAS 1232543-85-9); cyclopropanecarboxylic acid 3-(2,2-dichloroethenyl)-2,2-dimethyl-4-(1H-benzimidazol-2-yl)phenyl ester (known from CN 103524422 A) (CAS 1542271-46-4); (4aS)-7-chloro-2,5-dihydro-2-[[(methoxycarbonyl)[4-[(trifluoromethyl)thio]phenyl]amino]carbonyl]indeno [1,2-e][1,3,4]oxadiazine-4a(3H)-carboxylic acid methyl ester (known from CN 102391261 A) (CAS 1370358-69-2); 6-deoxy-3-O-ethyl-2,4-di-O-methyl-1-[N-[4-[1-[4-(1,1,2,2,2-pentafluoroethoxy)phenyl]-1H-1,2,4-triazole-3-yl]phenyl]carbamate]-α-L-mannopyranose (known from US 2014/0275503 A1) (CAS 1181213-14-8); 8-(2-cyclopropylmethoxy-4-trifluoromethylphenoxy)-3-(6-trifluoromethylpyridazin-3-yl)-3-azabicyclo[3.2.1]octane (CAS 1253850-56-4), (8-anti)-8-(2-cyclopropylmethoxy-4-trifluoromethylphenoxy)-3-(6-trifluoromethylpyridazin-3-yl)-3-azabicyclo[3.2.1]octane (CAS 933798-27-7), (8-syn)-8-(2-cyclopropylmethoxy-4-trifluoromethylphenoxy)-3-(6-trifluoromethylpyridazin-3-yl)-3-azabicyclo[3.2.1]octane (known from WO 2007040280 A1, WO 2007040282 A1) (CAS 934001-66-8) and N-[3-chloro-1-(3-pyridinyl)-1H-pyrazol-4-yl]-N-ethyl-3-[(3,3,3-trifluoropropyl)thio]propanamide (known from WO 2015/058021 A1, WO 2015/058028 A1) (CAS 1477919-27-9).

### Fungicides

The active ingredients specified herein by their common name are known and described, for example, in “Pesticide Manual” (16th Ed. British Crop Protection Council) or searchable on the internet (for example: http://www.alanwood.net/pesticides).

All the mixing components mentioned in classes (1) to (15), as the case may be, may form salts with suitable bases or acids if they are capable of doing so on the basis of their functional groups. All the fungicidal mixing components mentioned in classes (1) to (15), as the case may be, may include tautomeric forms.

1) Ergosterol biosynthesis inhibitors, for example (1.001) cyproconazole, (1.002) difenoconazole, (1.003) epoxiconazole, (1.004) fenhexamide, (1.005) fenpropidin, (1.006) fenpropimorph, (1.007) fenpyrazamine, (1.008) fluquinconazole, (1.009) flutriafol, (1.010) imazalil, (1.011) imazalil sulfate, (1.012) ipconazole, (1.013) metconazole, (1.014) myclobutanil, (1.015) paclobutrazole, (1.016) prochloraz, (1.017) propiconazole, (1.018) prothioconazole, (1.019) pyrisoxazole, (1.020) spiroxamine, (1.021) tebuconazole, (1.022) tetraconazole, (1.023) triadimenol, (1.024) tridemorph, (1.025) triticonazole, (1.026) (1R,2S,5S)-5-(4-chlorobenzyl)-2-(chloromethyl)-2-methyl-1-(1H-1,2,4-triazol-1-ylmethyl)cyclopentanol, (1.027) (1S,2R,5R)-5-(4-chlorobenzyl)-2-(chloromethyl)-2-methyl-1-(1H-1,2,4-triazol-1-ylmethyl)cyclopentanol, (1.028) (2R)-2-(1-chlorocyclopropyl)-4-[(1R)-2,2-dichlorocyclopropyl]-1-(1H-1,2,4-triazol-1-yl)butan-2-ol (1.029) (2R)-2-(1-chlorocyclopropyl)-4-[(1S)-2,2-dichlorocyclopropyl]-1-(1H-1,2,4-triazol-1-yl)butan-2-ol, (1.030) (2R)-2-[4-(4-chlorophenoxy)-2-(trifluoromethyl)phenyl]-1-(1H-1,2,4-triazol-1-yl)propan-2-ol, (1.031) (2S)-2-(1-chlorocyclopropyl)-4-[(1R)-2,2-dichlorocyclopropyl]-1-(1H-1,2,4-triazol-1-yl)butan-2-ol, (1.032) (2S)-2-(1-chlorocyclopropyl)-4-[(1S)-2,2-dichlorocyclopropyl]-1-(1H-1,2,4-triazol-1-yl)butan-2-ol, (1.033) (2S)-2-[4-(4-chlorophenoxy)-2-(trifluoromethyl)phenyl]-1-(1H-1,2,4-triazol-1-yl)propan-2-ol, (1.034) (R)-[3-(4-chloro-2-fluorophenyl)-5-(2,4-difluorophenyl)-1,2-oxazol-4-yl](pyridin-3-yl)methanol, (1.035) (S)-[3-(4-chloro-2-fluorophenyl)-5-(2,4-difluorophenyl)-1,2-oxazol-4-yl](pyridin-3-yl)methanol, (1.036) [3-(4-chloro-2-fluorophenyl)-5-(2,4-difluorophenyl)-1,2-oxazol-4-yl](pyridin-3-yl)methanol, (1.037) 1-({(2R,4S)-2-[2-chloro-4-(4-chlorophenoxy)phenyl]-4-methyl-1,3-dioxolan-2-yl}methyl)-1H-1,2,4-triazole, (1.038) 1-({(2S,4S)-2-[2-chloro-4-(4-chlorophenoxy)phenyl]-4-methyl-1,3-dioxolan-2-yl}methyl)-1H-1,2,4-triazole, (1.039) 1-{[3-(2-chlorophenyl)-2-(2,4-difluorophenyl)oxiran-2-yl]methyl}-1H-1,2,4-triazol-5-yl thiocyanate, (1.040) 1-{[rel(2R,3R)-3-(2-chlorophenyl)-2-(2,4-difluorophenyl)oxiran-2-yl]methyl}-1H-1,2,4-triazol-5-yl thiocyanate, (1.041) 1-{[rel(2R,3S)-3-(2-chlorophenyl)-2-(2,4-difluorophenyl)oxiran-2-yl]methyl}-1H-1,2,4-triazol-5-yl thiocyanate, (1.042) 2-[(2R,4R,5R)-1-(2,4-dichlorophenyl)-5-hydroxy-2,6,6-trimethylheptan-4-yl]-2,4-dihydro-3H-1,2,4-triazole-3-thione, (1.043) 2-[(2R,4R,5S)-1-(2,4-dichlorophenyl)-5-hydroxy-2,6,6-trimethylheptan-4-yl]-2,4-dihydro-3H-1,2,4-triazole-3-thione, (1.044) 2-[(2R,4S,5R)-1-(2,4-dichlorophenyl)-5-hydroxy-2,6,6-trimethylheptan-4-yl]-2,4-dihydro-3H-1,2,4-triazole-3-thione, (1.045) 2-[(2R,4S,5 S)-1-(2,4-dichlorophenyl)-5-hydroxy-2,6,6-trimethylheptan-4-yl]-2,4-dihydro-3H-1,2,4-triazole-3-thione, (1.046) 2-[(2S,4R,5R)-1-(2,4-dichlorophenyl)-5-hydroxy-2,6,6-trimethylheptan-4-yl]-2,4-dihydro-3H-1,2,4-triazole-3-thione, (1.047) 2-[(2S,4R,5 S)-1-(2,4-dichlorophenyl)-5-hydroxy-2,6,6-trimethylheptan-4-yl]-2,4-dihydro-3H-1,2,4-triazole-3-thione, (1.048) 2-[(2S,4S,5R)-1-(2,4-dichlorophenyl)-5-hydroxy-2,6,6-trimethylheptan-4-yl]-2,4-dihydro-3H-1,2,4-triazole-3-thione, (1.049) 2-[(2S,4S,5S)-1-(2,4-dichlorophenyl)-5-hydroxy-2,6,6-trimethylheptan-4-yl]-2,4-dihydro-3H-1,2,4-triazole-3-thione, (1.050) 2-[1-(2,4-dichlorophenyl)-5-hydroxy-2,6,6-trimethylheptan-4-yl]-2,4-dihydro-3H-1,2,4-triazole-3-thione, (1.051) 2-[2-chloro-4-(2,4-dichlorophenoxy)phenyl]-1-(1H-1,2,4-triazol-1-yl)propan-2-ol, (1.052) 2-[2-chloro-4-(4-chlorophenoxy)phenyl]-1-(1H-1,2,4-triazol-1-yl)butan-2-ol, (1.053) 2-[4-(4-chlorophenoxy)-2-(trifluoromethyl)phenyl]-1-(1H-1,2,4-triazol-1-yl)butan-2-ol, (1.054) 2-[4-(4-chlorophenoxy)-2-(trifluoromethyl)phenyl]-1-(1H-1,2,4-triazol-1-yl)pentan-2-ol, (1.055) 2-[4-(4-chlorophenoxy)-2-(trifluoromethyl)phenyl]-1-(1H-1,2,4-triazol-1 yl)propan-2-ol, (1.056) 2-{[3-(2-chlorophenyl)-2-(2,4-difluorophenyl)oxiran-2-yl]methyl}-2,4-dihydro-3H-1,2,4-triazole-3-thione, (1.057) 2-{[rel(2R,3R)-3-(2-chlorophenyl)-2-(2,4-difluorophenyl)oxiran-2-yl]methyl}-2,4-dihydro-3H-1,2,4-triazole-3-thione, (1.058) 2-{[rel(2R,3S)-3-(2-chlorophenyl)-2-(2,4-difluorophenyl)oxiran-2-yl]methyl}-2,4-dihydro-3H-1,2,4-triazole-3-thione, (1.059) 5-(4-chlorobenzyl)-2-(chloromethyl)-2-methyl-1-(1H-1,2,4-triazol-1-ylmethyl)cyclopentanol, (1.060) 5-(allylsulphanyl)-1-{[3-(2-chlorophenyl)-2-(2,4-difluorophenyl)oxiran-2-yl]methyl}-1H-1,2,4-triazole, (1.061) 5-(allylsulphanyl)-1-{[rel(2R,3R)-3-(2-chlorophenyl)-2-(2,4-difluorophenyl)oxiran-2-yl]methyl}-1H-1,2,4-triazole, (1.062) 5-(allylsulphanyl)-1-{[rel(2R,3S)-3-(2-chlorophenyl)-2-(2,4-difluorophenyl)oxiran-2-yl]methyl}-1H-1,2,4-triazole, (1.063) N′-(2,5-dimethyl-4-{[3-(1,1,2,2-tetrafluoroethoxy)phenyl]sulphanyl}phenyl)-N-ethyl-N-methylimidoformamide, (1.064) N′-(2,5-dimethyl-4-{[3-(2,2,2-trifluoroethoxy)phenyl]sulphanyl}phenyl)-N-ethyl-N-methylimidoformamide, (1.065) N′-(2,5-dimethyl-4-{[3-(2,2,3,3-tetrafluoropropoxy)phenyl]sulphanyl}phenyl)-N-ethyl-N-methylimidoformamide, (1.066) N′-(2,5-dimethyl-4-{[3-(pentafluoroethoxy)phenyl]sulphanyl}phenyl)-N-ethyl-N-methylimidoformamide, (1.067) N′-(2,5-dimethyl-4-{3-[(1,1,2,2-tetrafluoroethyl)sulphanyl]phenoxy}phenyl)-N-ethyl-N-methylimidoformamide, (1.068) N′-(2,5-dimethyl-4-{3-[(2,2,2-trifluoroethyl)sulphanyl]phenoxy}phenyl)-N-ethyl-N-methylimidoformamide, (1.069) N′-(2,5-dimethyl-4-{3-[(2,2,3,3-tetrafluoropropyl)sulphanyl]phenoxy}phenyl)-N-ethyl-N-methylimidoformamide, (1.070) N′-(2,5-dimethyl-4-{3-[(pentafluoroethyl)sulphanyl]phenoxy}phenyl)-N-ethyl-N-methylimidoformamide, (1.071) N′-(2,5-dimethyl-4-phenoxyphenyl)-N-ethyl-N-methylimidoformamide, (1.072) N′-(4-{[3-(difluoromethoxy)phenyl]sulphanyl}-2,5-dimethylphenyl)-N-ethyl-N-methylimidoformamide, (1.073) N′-(4-{3-[(difluoromethyl)sulphanyl]phenoxy}-2,5-dimethylphenyl)-N-ethyl-N-methylimidoformamide, (1.074) N′-[5-bromo-6-(2,3-dihydro-1H-inden-2-yloxy)-2-methylpyridin-3-yl]-N-ethyl-N-methylimidoformamide, (1.075) N′-{4-[(4,5-dichloro-1,3-thiazol-2-yl)oxy]-2,5-dimethylphenyl}-N-ethyl-N-methylimidoformamide, (1.076) N′-{5-bromo-6-[(1R)-1-(3,5-difluorophenyl)ethoxy]-2-methylpyridin-3-yl}-N-ethyl-N-methylimidoformamide, (1.077) N′-{5-bromo-6-[(1 S)-1-(3,5-difluorophenyl)ethoxy]-2-methylpyridin-3-yl}-N-ethyl-N-methylimidoformamide, (1.078) N′-{5-bromo-6-[(cis-4-isopropylcyclohexyl)oxy]-2-methylpyridin-3-yl}-N-ethyl-N-methylimidoformamide, (1.079) N′-{5-bromo-6-[(trans-4-isopropylcyclohexyl)oxy]-2-methylpyridin-3-yl}-N-ethyl-N-methylimidoformamide, (1.080) N′-{5-bromo-6-[1-(3,5-difluorophenyl)ethoxy]-2-methylpyridin-3-yl}-N-ethyl-N-methylimidoformamide.

2) Inhibitors of the respiratory chain in complex I or II, for example (2.001) benzovindiflupyr, (2.002) bixafen, (2.003) boscalid, (2.004) carboxin, (2.005) fluopyram, (2.006) flutolanil, (2.007) fluxapyroxad, (2.008) furametpyr, (2.009) isofetamid, (2.010) isopyrazam (anti-epimeric enantiomer 1R,4S,9S), (2.011) isopyrazam (anti-epimeric enantiomer 1S,4R,9R), (2.012) isopyrazam (anti-epimeric racemate 1RS,4SR,9SR), (2.013) isopyrazam (mixture of the syn-epimeric racemate 1RS,4SR,9RS and the anti-epimeric racemate 1RS,4SR,9SR), (2.014) isopyrazam (syn-epimeric enantiomer 1R,4S,9R), (2.015) isopyrazam (syn-epimeric enantiomer 1S,4R,9S), (2.016) isopyrazam (syn-epimeric racemate 1RS,4SR,9RS), (2.017) penflufen, (2.018) penthiopyrad, (2.019) pydiflumetofen, (2.020) pyraziflumid, (2.021) sedaxane, (2.022) 1,3-dimethyl-N-(1,1,3-trimethyl-2,3-dihydro-1H-inden-4-yl)-1H-pyrazole-4-carboxamide, (2.023) 1,3-dimethyl-N-[(3R)-1,1,3-trimethyl-2,3-dihydro-1H-inden-4-yl]-1H-pyrazole-4-carboxamide, (2.024) 1,3-dimethyl-N-[(3S)-1,1,3-trimethyl-2,3-dihydro-1H-inden-4-yl]-1H-pyrazole-4-carboxamide, (2.025) 1-methyl-3-(trifluoromethyl)-N-[2′-(trifluoromethyl)biphenyl-2-yl]-1H-pyrazole-4-carboxamide, (2.026) 2-fluoro-6-(trifluoromethyl)-N-(1,1,3-trimethyl-2,3-dihydro-1H-inden-4-yl)benzamide, (2.027) 3-(difluoromethyl)-1-methyl-N-(1,1,3-trimethyl-2,3-dihydro-1H-inden-4-yl)-1H-pyrazole-4-carboxamide, (2.028) 3-(difluoromethyl)-1-methyl-N-[(3R)-1,1,3-trimethyl-2,3-dihydro-1H-inden-4-yl]-1H-pyrazole-4-carboxamide, (2.029) 3-(difluoromethyl)-1-methyl-N-[(3S)-1,1,3-trimethyl-2,3-dihydro-1H-inden-4-yl]-1H-pyrazole-4-carboxamide, (2.030) 3-(difluoromethyl)-N-(7-fluoro-1,1,3-trimethyl-2,3-dihydro-1H-inden-4-yl)-1-methyl-1H-pyrazole-4-carboxamide, (2.031) 3-(difluoromethyl)-N-[(3R)-7-fluoro-1,1,3-trimethyl-2,3-dihydro-1H-inden-4-yl]-1-methyl-1H-pyrazole-4-carboxamide, (2.032) 3-(difluoromethyl)-N-[(3S)-7-fluoro-1,1,3-trimethyl-2,3-dihydro-1H-inden-4-yl]-1-methyl-1H-pyrazole-4-carboxamide, (2.033) 5,8-difluoro-N-[2-(2-fluoro-4-{[4-(trifluoromethyl)pyridin-2-yl]oxy}phenyl)ethyl]quinazolin-4-amine, (2.034) N-(2-cyclopentyl-5-fluorobenzyl)-N-cyclopropyl-3-(difluoromethyl)-5-fluoro-1-methyl-1H-pyrazole-4-carboxamide, (2.035) N-(2-tert-butyl-5-methylbenzyl)-N-cyclopropyl-3-(difluoromethyl)-5-fluoro-1-methyl-1H-pyrazole-4-carboxamide, (2.036) N-(2-tert-butylbenzyl)-N-cyclopropyl-3-(difluoromethyl)-5-fluoro-1-methyl-1H-pyrazole-4-carboxamide, (2.037) N-(5-chloro-2-ethylbenzyl)-N-cyclopropyl-3-(difluoromethyl)-5-fluoro-1-methyl-1H-pyrazole-4-carboxamide, (2.038) N-(5-chloro-2-isopropylbenzyl)-N-cyclopropyl-3-(difluoromethyl)-5-fluoro-1-methyl-1H-pyrazole-4-carboxamide, (2.039) N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide, (2.040) N-[(1 S,4R)-9-(dichloromethylene)-1,2,3,4-tetrahydro-1,4-methanonaphthalen-5-yl]-3-(difluoromethyl)-1-methyl-1H-pyrazole-4-carboxamide, (2.041) N-[1-(2,4-dichlorophenyl)-1-methoxypropan-2-yl]-3-(difluoromethyl)-1-methyl-1H-pyrazole-4-carboxamide, (2.042) N-[2-chloro-6-(trifluoromethyl)benzyl]-N-cyclopropyl-3-(difluoromethyl)-5-fluoro-1-methyl-1H-pyrazole-4-carboxamide, (2.043) N-[3-chloro-2-fluoro-6-(trifluoromethyl)benzyl]-N-cyclopropyl-3-(difluoromethyl)-5-fluoro-1-methyl-1H-pyrazole-4-carboxamide, (2.044) N-[5-chloro-2-(trifluoromethyl)benzyl]-N-cyclopropyl-3-(difluoromethyl)-5-fluoro-1-methyl-1H-pyrazole-4-carboxamide, (2.045) N-cyclopropyl-3-(difluoromethyl)-5-fluoro-1-methyl-N-[5-methyl-2-(trifluoromethyl)benzyl]-1H-pyrazole-4-carboxamide, (2.046) N-cyclopropyl-3-(difluoromethyl)-5-fluoro-N-(2-fluoro-6-isopropylbenzyl)-1-methyl-1H-pyrazole-4-carboxamide, (2.047) N-cyclopropyl-3-(difluoromethyl)-5-fluoro-N-(2-isopropyl-5-methylbenzyl)-1-methyl-1H-pyrazole-4-carboxamide, (2.048) N-cyclopropyl-3-(difluoromethyl)-5-fluoro-N-(2-isopropylbenzyl)-1-methyl-1H-pyrazole-4-carbothioamide, (2.049) N-cyclopropyl-3-(difluoromethyl)-5-fluoro-N-(2-isopropylbenzyl)-1-methyl-1H-pyrazole-4-carboxamide, (2.050) N-cyclopropyl-3-(difluoromethyl)-5-fluoro-N-(5-fluoro-2-isopropylbenzyl)-1-methyl-1H-pyrazole-4-carboxamide, (2.051) N-cyclopropyl-3-(difluoromethyl)-N-(2-ethyl-4,5-dimethylbenzyl)-5-fluoro-1-methyl-1H-pyrazole-4-carboxamide, (2.052) N-cyclopropyl-3-(difluoromethyl)-N-(2-ethyl-5-fluorobenzyl)-5-fluoro-1-methyl-1H-pyrazole-4-carboxamide, (2.053) N-cyclopropyl-3-(difluoromethyl)-N-(2-ethyl-5-methylbenzyl)-5-fluoro-1-methyl-1H-pyrazole-4-carboxamide, (2.054) N-cyclopropyl-N-(2-cyclopropyl-5-fluorobenzyl)-3-(difluoromethyl)-5-fluoro-1-methyl-1H-pyrazole-4-carboxamide, (2.055) N-cyclopropyl-N-(2-cyclopropyl-5-methylbenzyl)-3-(difluoromethyl)-5-fluoro-1-methyl-1H-pyrazole-4-carboxamide, (2.056) N-cyclopropyl-N-(2-cyclopropylbenzyl)-3-(difluoromethyl)-5-fluoro-1-methyl-1H-pyrazole-4-carboxamide.

3) Inhibitors of the respiratory chain in complex III, for example (3.001) ametoctradin, (3.002) amisulbrom, (3.003) azoxystrobin, (3.004) coumethoxystrobin, (3.005) coumoxystrobin, (3.006) cyazofamid, (3.007) dimoxystrobin, (3.008) enoxastrobin, (3.009) famoxadon, (3.010) fenamidon, (3.011) flufenoxystrobin, (3.012) fluoxastrobin, (3.013) kresoxim-methyl, (3.014) metominostrobin, (3.015) orysastrobin, (3.016) picoxystrobin, (3.017) pyraclostrobin, (3.018) pyrametostrobin, (3.019) pyraoxystrobin, (3.020) trifloxystrobin (3.021) (2E)-2-{2-[({[(1E)-1-(3-{[(E)-1-fluoro-2-phenylvinyl]oxy}phenyl)ethylidene]amino}oxy)methyl]phenyl}-2-(methoxyimino)-N-methylacetamide, (3.022) (2E,3Z)-5-{[1-(4-chlorophenyl)-1H-pyrazol-3-yl]oxy}-2-(methoxyimino)-N,3-dimethylpent-3-enamide, (3.023) (2R)-2-{2-[(2,5-dimethylphenoxy)methyl]phenyl}-2-methoxy-N-methylacetamide, (3.024) (2S)-2-{2-[(2,5-dimethylphenoxy)methyl]phenyl}-2-methoxy-N-methylacetamide, (3.025) (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, (3.026) 2-{2-[(2,5-dimethylphenoxy)methyl]phenyl}-2-methoxy-N-methylacetamide, (3.027) N-(3-ethyl-3,5,5-trimethylcyclohexyl)-3-formamido-2-hydroxybenzamide, (3.028) (2E,3Z)-5-{[1-(4-chloro-2-fluorophenyl)-1H-pyrazol-3-yl]oxy}-2-(methoxyimino)-N,3-dimethylpent-3-enamide.

4) Mitosis and cell division inhibitors, for example (4.001) carbendazim, (4.002) diethofencarb, (4.003) ethaboxam, (4.004) fluopicolid, (4.005) pencycuron, (4.006) thiabendazole, (4.007) thiophanate-methyl, (4.008) zoxamide, (4.009) 3-chloro-4-(2,6-difluorophenyl)-6-methyl-5-phenylpyridazine, (4.010) 3-chloro-5-(4-chlorophenyl)-4-(2,6-difluorophenyl)-6-methylpyridazine, (4.011) 3-chloro-5-(6-chloropyridin-3-yl)-6-methyl-4-(2,4,6-trifluorophenyl)pyridazine, (4.012) 4-(2-bromo-4-fluorophenyl)-N-(2,6-difluorophenyl)-1,3-dimethyl-1H-pyrazol-5-amine, (4.013) 4-(2-bromo-4-fluorophenyl)-N-(2-bromo-6-fluorophenyl)-1,3-dimethyl-1H-pyrazol-5-amine, (4.014) 4-(2-bromo-4-fluorophenyl)-N-(2-bromophenyl)-1,3-dimethyl-1H-pyrazol-5-amine, (4.015) 4-(2-bromo-4-fluorophenyl)-N-(2-chloro-6-fluorophenyl)-1,3-dimethyl-1H-pyrazol-5-amine, (4.016) 4-(2-bromo-4-fluorophenyl)-N-(2-chlorophenyl)-1,3-dimethyl-1H-pyrazol-5-amine, (4.017) 4-(2-bromo-4-fluorophenyl)-N-(2-fluorophenyl)-1,3-dimethyl-1H-pyrazol-5-amine, (4.018) 4-(2-chloro-4-fluorophenyl)-N-(2,6-difluorophenyl)-1,3-dimethyl-1H-pyrazol-5-amine, (4.019) 4-(2-chloro-4-fluorophenyl)-N-(2-chloro-6-fluorophenyl)-1,3-dimethyl-1H-pyrazol-5-amine, (4.020) 4-(2-chloro-4-fluorophenyl)-N-(2-chlorophenyl)-1,3-dimethyl-1H-pyrazol-5-amine, (4.021) 4-(2-chloro-4-fluorophenyl)-N-(2-fluorophenyl)-1,3-dimethyl-1H-pyrazol-5-amine, (4.022) 4-(4-chlorophenyl)-5-(2,6-difluorophenyl)-3,6-dimethylpyridazine, (4.023) N-(2-bromo-6-fluorophenyl)-4-(2-chloro-4-fluorophenyl)-1,3-dimethyl-1H-pyrazol-5-amine, (4.024) N-(2-bromophenyl)-4-(2-chloro-4-fluorophenyl)-1,3-dimethyl-1H-pyrazol-5-amine, (4.025) N-(4-chloro-2,6-difluorophenyl)-4-(2-chloro-4-fluorophenyl)-1,3-dimethyl-1H-pyrazol-5-amine.

5) Compounds having capacity for multisite activity, for example (5.001) Bordeaux mixture, (5.002) captafol, (5.003) captan, (5.004) chlorthalonil, (5.005) copper hydroxide, (5.006) copper naphthenate, (5.007) copper oxide, (5.008) copper oxychloride, (5.009) copper(2+) sulphate, (5.010) dithianon, (5.011) dodin, (5.012) folpet, (5.013) mancozeb, (5.014) maneb, (5.015) metiram, (5.016) zinc metiram, (5.017) copper oxine, (5.018) propineb, (5.019) sulphur and sulphur preparations including calcium polysulphide, (5.020) thiram, (5.021) zineb, (5.022) ziram.

6) Compounds capable of triggering host defence, for example (6.001) acibenzolar-S-methyl, (6.002) isotianil, (6.003) probenazole, (6.004) tiadinil.

7) Amino acid and/or protein biosynthesis inhibitors, for example (7.001) cyprodinil, (7.002) kasugamycin, (7.003) kasugamycin hydrochloride hydrate, (7.004) oxytetracycline, (7.005) pyrimethanil, (7.006) 3-(5-fluoro-3,3,4,4-tetramethyl-3,4-dihydroisoquinolin-1-yl)quinoline.

(8) ATP production inhibitors, for example (8.001) silthiofam.

9) Cell wall synthesis inhibitors, for example (9.001) benthiavalicarb, (9.002) dimethomorph, (9.003) flumorph, (9.004) iprovalicarb, (9.005) mandipropamid, (9.006) pyrimorph, (9.007) valifenalate, (9.008) (2E)-3-(4-tert-butylphenyl)-3-(2-chloropyridin-4-yl)-1-(morpholin-4-yl)prop-2-en-1-one, (9.009) (2Z)-3-(4-tert-butylphenyl)-3-(2-chloropyridin-4-yl)-1-(morpholin-4-yl)prop-2-en-1-one.

10) Lipid and membrane synthesis inhibitors, for example (10.001) propamocarb, (10.002) propamocarb hydrochloride, (10.003) tolclofos-methyl.

11) Melanin biosynthesis inhibitors, for example (11.001) tricyclazole, (11.002) 2,2,2-trifluoroethyl {3-methyl-1-[(4-methylbenzoyl)amino]butan-2-yl}carbamate.

12) Nucleic acid synthesis inhibitors, for example (12.001) benalaxyl, (12.002) benalaxyl-M (kiralaxyl), (12.003) metalaxyl, (12.004) metalaxyl-M (mefenoxam).

13) Signal transduction inhibitors, for example (13.001) fludioxonil, (13.002) iprodione, (13.003) procymidone, (13.004) proquinazid, (13.005) quinoxyfen, (13.006) vinclozolin.

14) Compounds that can act as uncouplers, for example (14.001) fluazinam, (14.002) meptyldinocap.

15) Further compounds, for example (15.001) abscisic acid, (15.002) benthiazole, (15.003) bethoxazin, (15.004) capsimycin, (15.005) carvone, (15.006) chinomethionat, (15.007) cufraneb, (15.008) cyflufenamid, (15.009) cymoxanil, (15.010) cyprosulfamide, (15.011) flutianil, (15.012) fosetyl-aluminium, (15.013) fosetyl-calcium, (15.014) fosetyl-sodium, (15.015) methyl isothiocyanate, (15.016) metrafenon, (15.017) mildiomycin, (15.018) natamycin, (15.019) nickel dimethyldithiocarbamate, (15.020) nitrothal-isopropyl, (15.021) oxamocarb, (15.022) oxathiapiprolin, (15.023) oxyfenthiin, (15.024) pentachlorophenol and salts, (15.025) phosphonic acid and salts thereof, (15.026) propamocarb-fosetylate, (15.027) pyriofenone (chlazafenone) (15.028) tebufloquin, (15.029) tecloftalam, (15.030) tolnifanide, (15.031) 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.032) 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, (15.033) 2-(6-benzylpyridin-2-yl)quinazoline, (15.034) 2,6-dimethyl-1H,5H-[1,4]dithiino[2,3-c:5,6-c′]dipyrrole-1,3,5,7(2H,6H)-tetrone, (15.035) 2-[3,5-bis(difluoromethyl)-1H-pyrazol-1-yl]-1-[4-(4-{5-[2-(prop-2-yn-1-yloxy)phenyl]-4,5-dihydro-1,2-oxazol-3-yl}-1,3-thiazol-2-yl)piperidin-1-yl]ethanone, (15.036) 2-[3,5-bis(difluoromethyl)-1H-pyrazol-1-yl]-1-[4-(4-{5-[2-chloro-6-(prop-2-yn-1-yloxy)phenyl]-4,5-dihydro-1,2-oxazol-3-yl}-1,3-thiazol-2-yl)piperidin-1-yl]ethanone, (15.037) 2-[3,5-bis(difluoromethyl)-1H-pyrazol-1-yl]-1-[4-(4-{5-[2-fluoro-6-(prop-2-yn-1-yloxy)phenyl]-4,5-dihydro-1,2-oxazol-3-yl}-1,3-thiazol-2-yl)piperidin-1-yl]ethanone, (15.038) 2-[6-(3-fluoro-4-methoxyphenyl)-5-methylpyridin-2-yl]quinazoline, (15.039) 2-{(5R)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.040) 2-{(5S)-3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.041) 2-{2-[(7,8-difluoro-2-methylquinolin-3-yl)oxy]-6-fluorophenyl}propan-2-ol, (15.042) 2-{2-fluoro-6-[(8-fluoro-2-methylquinolin-3-yl)oxy]phenyl}propan-2-ol, (15.043) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}-3-chlorophenyl methanesulphonate, (15.044) 2-{3-[2-(1-{[3,5-bis(difluoromethyl)-1H-pyrazol-1-yl]acetyl}piperidin-4-yl)-1,3-thiazol-4-yl]-4,5-dihydro-1,2-oxazol-5-yl}phenyl methanesulphonate, (15.045) 2-phenylphenol and salts thereof, (15.046) 3-(4,4,5-trifluoro-3,3-dimethyl-3,4-dihydroisoquinolin-1-yl)quinoline, (15.047) 3-(4,4-difluoro-3,3-dimethyl-3,4-dihydroisoquinolin-1-yl)quinoline, (15.048) 4-amino-5-fluoropyrimidin-2-ol (tautomeric form: 4-amino-5-fluoropyrimidin-2(1H)-one), (15.049) 4-oxo-4-[(2-phenylethyl)amino]butyric acid, (15.050) 5-amino-1,3,4-thiadiazole-2-thiol, (15.051) 5-chloro-N′-phenyl-N′-(prop-2-yn-1-yl)thiophene 2-sulphonohydrazide, (15.052) 5-fluoro-2-[(4-fluorobenzyl)oxy]pyrimidin-4-amine, (15.053) 5-fluoro-2-[(4-methylbenzyl)oxy]pyrimidin-4-amine, (15.054) 9-fluoro-2,2-dimethyl-5-(quinolin-3-yl)-2,3-dihydro-1,4-benzoxazepine, (15.055) but-3-yn-1-yl {6-[({[(Z)-(1-methyl-1H-tetrazol-5-yl)(phenyl)methylene]amino}oxy)methyl]pyridin-2-yl}carbamate, (15.056) ethyl (2Z)-3-amino-2-cyano-3-phenylacrylate, (15.057) phenazine-1-carboxylic acid, (15.058) propyl 3,4,5-trihydroxybenzoate, (15.059) quinolin-8-ol, (15.060) quinolin-8-ol sulphate (2:1), (15.061) tert-butyl {6-[({[(1-methyl-1H-tetrazol-5-yl)(phenyl)methylene]amino}oxy)methyl]pyridin-2-yl}carbamate.

### Biological Pesticides as Mixing Components

The compounds of the formula (I) can be combined with biological pesticides.

Biological pesticides especially include bacteria, fungi, yeasts, plant extracts and products formed by microorganisms, including proteins and secondary metabolites.

Biological pesticides include bacteria such as spore-forming bacteria, root-colonizing bacteria and bacteria which act as biological insecticides, fungicides or nematicides.

Examples of such bacteria which are used or can be used as biological pesticides are:

Bacillus amyloliquefaciens, strain FZB42 (DSM 231179), or Bacillus cereus, especially B. cereus strain CNCM 1-1562 or Bacillus firmus, strain 1-1582 (Accession number CNCM 1-1582) or Bacillus pumilus, especially strain GB34 (Accession No. ATCC 700814) and strain QST2808 (Accession No. NRRL B-30087), or Bacillus subtilis, especially strain GB03 (Accession No. ATCC SD-1397), or Bacillus subtilis strain QST713 (Accession No. NRRL B-21661) or Bacillus subtilis strain OST 30002 (Accession No. NRRL B-50421), Bacillus thuringiensis, especially B. thuringiensis subspecies israelensis (serotype H-14), strain AM65-52 (Accession No. ATCC 1276), or B. thuringiensis subsp. aizawai, especially strain ABTS-1857 (SD-1372), or B. thuringiensis subsp. kurstaki strain HD-1, or B. thuringiensis subsp. tenebrionis strain NB 176 (SD-5428), Pasteuria penetrans, Pasteuria spp. (Rotylenchulus reniformis nematode)-PR3 (Accession Number ATCC SD-5834), Streptomyces microflavus strain AQ6121 (=QRD 31.013, NRRL B-50550), Streptomyces galbus strain AQ 6047 (Accession Number NRRL 30232).

Examples of fungi and yeasts which are used or can be used as biological pesticides are:

Beauveria bassiana, in particular strain ATCC 74040, Coniothyrium minitans, in particular strain CON/M/91-8 (Accession No. DSM-9660), Lecanicillium spp., in particular strain HRO LEC 12, Lecanicillium lecanii (formerly known as Verticillium lecanii), in particular strain KV01, Metarhizium anisopliae, in particular strain F52 (DSM3884/ATCC 90448), Metschnikowia fructicola, in particular strain NRRL Y-30752, Paecilomyces fumosoroseus (new: Isaria fumosorosea), in particular strain IFPC 200613, or strain Apopka 97 (Accession No. ATCC 20874), Paecilomyces lilacinus, in particular P. lilacinus strain 251 (AGAL 89/030550), Talaromyces flavus, in particular strain V117b, Trichoderma atroviride, in particular strain SC1 (Accession Number CBS 122089), Trichoderma harzianum, in particular T. harzianum rifai T39 (Accession Number CNCM 1-952).

Examples of viruses which are used or can be used as biological pesticides are:

Adoxophyes orana (summer fruit tortrix) granulosis virus (GV), Cydia pomonella (codling moth) granulosis virus (GV), Helicoverpa armigera (cotton bollworm) nuclear polyhedrosis virus (NPV), Spodoptera exigua (beet armyworm) mNPV, Spodoptera frugiperda (fall armyworm) mNPV, Spodoptera littoralis (African cotton leafworm) NPV.

Also included are bacteria and fungi which are added as ‘inoculant’ to plants or plant parts or plant organs and which, by virtue of their particular properties, promote plant growth and plant health. Examples include:

Agrobacterium spp., Azorhizobium caulinodans, Azospirillum spp., Azotobacter spp., Bradyrhizobium spp., Burkholderia spp., especially Burkholderia cepacia (formerly known as Pseudomonas cepacia), Gigaspora spp., or Gigaspora monosporum, Glomus spp., Laccaria spp., Lactobacillus buchneri, Paraglomus spp., Pisolithus tinctorus, Pseudomonas spp., Rhizobium spp., especially Rhizobium trifolii, Rhizopogon spp., Scleroderma spp., Suillus spp., Streptomyces spp.

Examples of plant extracts and products formed by microorganisms, including proteins and secondary metabolites, which are used or can be used as biological pesticides are:

Allium sativum, Artemisia absinthium, azadirachtin, Biokeeper WP, Cassia nigricans, Celastrus angulatus, Chenopodium anthelminticum, chitin, Armour-Zen, Dryopteris filix-mas, Equisetum arvense, Fortune Aza, Fungastop, Heads Up (Chenopodium quinoa saponin extract), pyrethrum/pyrethrins, Quassia amara, Quercus, Quillaja, Regalia, “Requiem™ Insecticide”, rotenone, ryania/ryanodine, Symphytum officinale, Tanacetum vulgare, thymol, Triact 70, TriCon, Tropaeulum majus, Urtica dioica, Veratrin, Viscum album, Brassicaceae extract, especially oilseed rape powder or mustard powder.

### Safeners as Mixing Components

The compounds of the formula (I) can be combined with safeners, for example benoxacor, cloquintocet (-mexyl), cyometrinil, cyprosulfamide, dichlormid, fenchlorazole (-ethyl), fenclorim, flurazole, fluxofenim, furilazole, isoxadifen (-ethyl), mefenpyr (-diethyl), naphthalic anhydride, oxabetrinil, 2-methoxy-N-({4-[(methylcarbamoyl)amino]phenyl}sulphonyl)benzamide (CAS 129531-12-0), 4-(dichloroacetyl)-1-oxa-4-azaspiro[4.5]decane (CAS 71526-07-3), 2,2,5-trimethyl-3-(dichloroacetyl)-1,3-oxazolidine (CAS 52836-31-4).

### Plants and Plant Parts

All plants and plant parts can be treated in accordance with the invention. Plants are understood here to mean all plants and populations of plants, such as desirable and undesirable wild plants or crop plants (including naturally occurring crop plants), for example cereals (wheat, rice, triticale, barley, rye, oats), maize, soya beans, potatoes, sugar beet, sugar cane, tomatoes, bell peppers, cucumbers, melons, carrots, water melons, onions, lettuce, spinach, leeks, beans, Brassica oleracea (e.g. cabbage) and other vegetable species, cotton, tobacco, oilseed rape, and also fruit plants (the fruits being apples, pears, citrus fruits and grapes). Crop plants may be plants which can be obtained by conventional breeding and optimization methods or by biotechnological and genetic engineering methods or combinations of these methods, including the transgenic plants and including the plant cultivars which are protectable or non-protectable by plant breeders' rights. Plants shall be understood to mean all development stages such as seed, seedlings, young (immature) plants, up to and including mature plants. Plant parts shall be understood to mean all parts and organs of the plants above and below ground, such as shoot, leaf, flower and root, examples given being leaves, needles, stalks, stems, flowers, fruit bodies, fruits and seeds, and also roots, tubers and rhizomes. Plant parts also include harvested plants or harvested plant parts and vegetative and generative propagation material, for example cuttings, tubers, rhizomes, slips and seeds.

The treatment according to the invention of the plants and parts of plants with the compounds of the formula (I) is effected directly or by allowing the compounds to act on the surroundings, the habitat or the storage space thereof by the customary treatment methods, for example by dipping, spraying, evaporating, fogging, scattering, painting on, injecting, and, in the case of propagation material, especially in the case of seeds, also by applying one or more coats.

As already mentioned above, it is possible to treat all plants and their parts in accordance with the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (genetically modified organisms), and parts thereof are treated. The term “parts” or “parts of plants” or “plant parts” has been explained above. Particular preference is given in accordance with the invention to treating plants of the respective commercially customary plant cultivars or those that are in use. Plant cultivars are understood to mean plants having new properties (“traits”) and which have been obtained by conventional breeding, by mutagenesis or by recombinant DNA techniques. They may be cultivars, varieties, biotypes or genotypes.

### Transgenic Plants, Seed Treatment and Integration Events

The preferred transgenic plants or plant cultivars (those obtained by genetic engineering) which are to be treated in accordance with the invention include all plants which, through the genetic modification, received genetic material which imparts particular advantageous useful properties (“traits”) to these plants. Examples of such properties are better plant growth, increased tolerance to high or low temperatures, increased tolerance to drought or to levels of water or soil salinity, enhanced flowering performance, easier harvesting, accelerated ripening, higher harvest yields, higher quality and/or higher nutritional value of the harvested products, better capability for storage and/or processability of the harvested products. Further and particularly emphasized examples of such properties are increased resistance of the plants to animal and microbial pests, such as insects, arachnids, nematodes, mites, slugs and snails, owing, for example, to toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof), and also increased resistance of the plants to phytopathogenic fungi, bacteria and/or viruses caused, for example, by systemic acquired resistance (SAR), systemin, phytoalexins, elicitors and resistance genes and correspondingly expressed proteins and toxins, and also increased tolerance of the plants to certain active herbicidal ingredients, for example imidazolinones, sulphonylureas, glyphosate or phosphinothricin (for example the “PAT” gene). The genes which impart the desired properties (“traits”) in question may also be present in combinations with one another in the transgenic plants. Examples of transgenic plants mentioned include the important crop plants, such as cereals (wheat, rice, triticale, barley, rye, oats), maize, soya beans, potatoes, sugar beet, sugar cane, tomatoes, peas and other types of vegetable, cotton, tobacco, oilseed rape and also fruit plants (the fruits being apples, pears, citrus fruits and grapevines), particular emphasis being given to maize, soya beans, wheat, rice, potatoes, cotton, sugar cane, tobacco and oilseed rape. Properties (“traits”) which are particularly emphasized are the increased resistance of the plants to insects, arachnids, nematodes and slugs and snails.

### Crop Protection—Types of Treatment

The plants and plant parts are treated with the compounds of the formula (I) directly or by action on their surroundings, habitat or storage space using customary treatment methods, for example by dipping, spraying, atomizing, irrigating, evaporating, dusting, fogging, broadcasting, foaming, painting, spreading-on, injecting, watering (drenching), drip irrigating and, in the case of propagation material, in particular in the case of seed, additionally by dry seed treatment, liquid seed treatment, slurry treatment, by incrusting, by coating with one or more coats, etc. It is furthermore possible to apply the compounds of the formula (I) by the ultra-low volume method or to inject the application form or the compound of the formula (I) itself into the soil.

A preferred direct treatment of the plants is foliar application, meaning that the compounds of the formula (I) are applied to the foliage, in which case the treatment frequency and the application rate should be adjusted according to the level of infestation with the pest in question.

In the case of systemically active ingredients, the compounds of the formula (I) also access the plants via the root system. The plants are then treated by the action of the compounds of the formula (I) on the habitat of the plant. This can be accomplished, for example, by drenching, or by mixing into the soil or the nutrient solution, meaning that the locus of the plant (e.g. soil or hydroponic systems) is impregnated with a liquid form of the compounds of the formula (I), or by soil application, meaning that the compounds of the formula (I) according to the invention are introduced in solid form (e.g. in the form of granules) into the locus of the plants. In the case of paddy rice crops, this can also be accomplished by metering the compound of the formula (I) in a solid application form (for example as granules) into a flooded paddy field.

### Seed Treatment

The control of animal pests by the treatment of the seed of plants has long been known and is the subject of constant improvements. Nevertheless, the treatment of seed entails a series of problems which cannot always be solved in a satisfactory manner. Thus, it is desirable to develop methods for protecting the seed and the germinating plant which dispense with, or at least reduce considerably, the additional application of pesticides during storage, after sowing or after emergence of the plants. It is additionally desirable to optimize the amount of active ingredient used so as to provide optimum protection for the seed and the germinating plant from attack by animal pests, but without damage to the plant itself by the active ingredient used. In particular, methods for the treatment of seed should also take account of the intrinsic insecticidal or nematicidal properties of pest-resistant or -tolerant transgenic plants in order to achieve optimal protection of the seed and also the germinating plant with a minimum expenditure on pesticides.

The present invention therefore in particular also relates to a method for the protection of seed and germinating plants from attack by pests, by treating the seed with one of the compounds of the formula (I). The method according to the invention for protecting seed and germinating plants against attack by pests further comprises a method in which the seed is treated simultaneously in one operation or sequentially with a compound of the formula (I) and a mixing component. It further also comprises a method where the seed is treated at different times with a compound of the formula (I) and a mixing component.

The invention likewise relates to the use of the compounds of the formula (I) for the treatment of seed for protecting the seed and the resulting plant from animal pests.

The invention further relates to seed which has been treated with a compound of the formula (I) according to the invention for protection from animal pests. The invention also relates to seed which has been treated simultaneously with a compound of the formula (I) and a mixing component. The invention further relates to seed which has been treated at different times with a compound of the formula (I) and a mixing component. In the case of seed which has been treated at different times with a compound of the formula (I) and a mixing component, the individual substances may be present on the seed in different layers. In this case, the layers comprising a compound of the formula (I) and mixing components may optionally be separated by an intermediate layer. The invention also relates to seed in which a compound of the formula (I) and a mixing component have been applied as part of a coating or as a further layer or further layers in addition to a coating.

The invention further relates to seed which, after the treatment with a compound of the formula (I), is subjected to a film-coating process to prevent dust abrasion on the seed.

One of the advantages that occur when a compound of the formula (I) acts systemically is that the treatment of the seed protects not only the seed itself but also the plants resulting therefrom, after emergence, from animal pests. In this way, the immediate treatment of the crop at the time of sowing or shortly thereafter can be dispensed with.

A further advantage is that the treatment of the seed with a compound of the formula (I) can enhance germination and emergence of the treated seed.

It is likewise considered to be advantageous that compounds of the formula (I) can especially also be used for transgenic seed.

Furthermore, compounds of the formula (I) can be employed in combination with compositions of signalling technology, leading to better colonization by symbionts such as, for example, rhizobia, mycorrhizae and/or endophytic bacteria or fungi, and/or to optimized nitrogen fixation.

The compounds of the formula (I) are suitable for protection of seed of any plant variety which is used in agriculture, in the greenhouse, in forests or in horticulture. More particularly, this is the seed of cereals (for example wheat, barley, rye, millet and oats), maize, cotton, soya beans, rice, potatoes, sunflowers, coffee, tobacco, canola, oilseed rape, beets (for example sugar beets and fodder beets), peanuts, vegetables (for example tomatoes, cucumbers, beans, cruciferous vegetables, onions and lettuce), fruit plants, lawns and ornamental plants. Of particular significance is the treatment of the seed of cereals (such as wheat, barley, rye and oats), maize, soya beans, cotton, canola, oilseed rape, vegetables and rice.

As already mentioned above, the treatment of transgenic seed with a compound of the formula (I) is also of particular importance. This involves the seed of plants which generally contain at least one heterologous gene which controls the expression of a polypeptide having insecticidal and/or nematicidal properties in particular. The heterologous genes in transgenic seed may originate from microorganisms such as Bacillus, Rhizobium, Pseudomonas, Serratia, Trichoderma, Clavibacter, Glomus or Gliocladium. The present invention is particularly suitable for treatment of transgenic seed which comprises at least one heterologous gene originating from Bacillus sp. The heterologous gene is more preferably derived from Bacillus thuringiensis.

In the context of the present invention, the compound of the formula (I) is applied to the seed. The seed is preferably treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content which allows storage. Alternatively, it is also possible to use seed which, after drying, has been treated with, for example, water and then dried again, for example priming. In the case of rice seed, it is also possible to use seed which has been soaked, for example in water, until it reaches a certain stage of the rice embryo (“pigeon breast stage”) which results in stimulation of germination and more uniform emergence.

When treating the seed, care must generally be taken that the amount of the compound of the formula (I) applied to the seed and/or the amount of further additives is chosen in such a way that the germination of the seed is not adversely affected, or that the resulting plant is not damaged. This has to be ensured particularly in the case of active ingredients which can exhibit phytotoxic effects at certain application rates.

In general, the compounds of the formula (I) are applied to the seed in the form of a suitable formulation. Suitable formulations and processes for seed treatment are known to the person skilled in the art.

The compounds of the formula (I) can be converted to the customary seed-dressing formulations, such as solutions, emulsions, suspensions, powders, foams, slurries or other coating compositions for seed, and also ULV formulations.

These formulations are prepared in a known manner, by mixing the compounds of the formula (I) with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of active agrochemical ingredients. Usable with preference are alkyl naphthalenesulphonates, such as diisopropyl or diisobutyl naphthalenesulphonates.

Suitable dispersants and/or emulsifiers which may be present in the seed-dressing formulations usable in accordance with the invention are all nonionic, anionic and cationic dispersants customary for the formulation of active agrochemical ingredients. Nonionic or anionic dispersants or mixtures of nonionic or anionic dispersants can be used with preference. Suitable nonionic dispersants especially include ethylene oxide/propylene oxide block polymers, alkylphenol polyglycol ethers and tristyrylphenol polyglycol ethers, and the phosphated or sulphated derivatives thereof. Suitable anionic dispersants are especially lignosulphonates, polyacrylic acid salts and arylsulphonate-formaldehyde condensates.

Antifoams which may be present in the seed-dressing formulations usable in accordance with the invention are all foam-inhibiting substances customary for the formulation of active agrochemical ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances which can be used for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

Gibberellins which may be present in the seed-dressing formulations usable in accordance with the invention are preferably the gibberellins A1, A3 (=gibberellic acid), A4 and A7; particular preference is given to using gibberellic acid. The gibberellins are known (cf. R. Wegler “Chemie der Pflanzenschutz- and Schädlingsbekampfungsmittel”, vol. 2, Springer Verlag, 1970, pp. 401-412).

The seed-dressing formulations usable in accordance with the invention can be used to treat a wide variety of different kinds of seed, either directly or after prior dilution with water. For instance, the concentrates or the preparations obtainable therefrom by dilution with water can be used to dress the seed of cereals, such as wheat, barley, rye, oats and triticale, and also the seed of maize, rice, oilseed rape, peas, beans, cotton, sunflowers, soya beans and beets, or else a wide variety of different vegetable seed. The seed-dressing formulations usable in accordance with the invention, or the dilute use forms thereof, can also be used to dress seed of transgenic plants.

For the treatment of seed with the seed-dressing formulations usable in accordance with the invention, or the use forms prepared therefrom through the addition of water, all mixing units usable customarily for the seed dressing are useful. Specifically, the procedure in seed dressing is to place the seed into a mixer in batchwise or continuous operation, to add the particular desired amount of seed-dressing formulations, either as such or after prior dilution with water, and to mix until the formulation is distributed homogeneously on the seed. If appropriate, this is followed by a drying operation.

The application rate of the seed-dressing formulations usable in accordance with the invention can be varied within a relatively wide range. It is guided by the particular content of the compounds of the formula (I) in the formulations and by the seed. The application rates of the compound of the formula (I) are generally between 0.001 and 50 g per kilogram of seed, preferably between 0.01 and 15 g per kilogram of seed.

### Animal Health

In the animal health field, i.e. the field of veterinary medicine, the compounds of the formula (I) are active against animal parasites, in particular ectoparasites or endoparasites. The term “endoparasite” includes especially helminths and protozoa, such as coccidia. Ectoparasites are typically and preferably arthropods, especially insects or acarids.

In the field of veterinary medicine, the compounds of the formula (I) having favourable endotherm toxicity are suitable for controlling parasites which occur in animal breeding and animal husbandry in livestock, breeding animals, zoo animals, laboratory animals, experimental animals and domestic animals. They are active against all or specific stages of development of the parasites.

Agricultural livestock include, for example, mammals, such as sheep, goats, horses, donkeys, camels, buffalo, rabbits, reindeer, fallow deer and especially cattle and pigs; or poultry such as turkeys, ducks, geese and especially chickens; or fish or crustaceans, for example in aquaculture; or, as the case may be, insects such as bees.

Domestic animals include, for example, mammals, such as hamsters, guinea pigs, rats, mice, chinchillas, ferrets, and particularly dogs, cats, caged birds; reptiles, amphibians or aquarium fish.

In a specific embodiment, the compounds of the formula (I) are administered to mammals.

In another specific embodiment, the compounds of the formula (I) are administered to birds, namely caged birds or particularly poultry.

Use of the compounds of the formula (I) for the control of animal parasites is intended to reduce or prevent illness, cases of death and reductions in performance (in the case of meat, milk, wool, hides, eggs, honey and the like), such that more economical and simpler animal husbandry is enabled and better animal well-being is achievable.

In relation to the field of animal health, the term “control” or “controlling” in the present context means that the compounds of the formula (I) are effective in reducing the incidence of the particular parasite in an animal infected with such parasites to an innocuous degree. More specifically, “controlling” in the present context means that the compounds of the formula (I) kill the respective parasite, inhibit its growth, or inhibit its proliferation.

The arthropods include, for example, but are not limited to,

from the order of Anoplurida, for example, Haematopinus spp., Linognathus spp., Pediculus spp., Phtirus spp. and Solenopotes spp.;
from the order of Mallophagida and the suborders Amblycerina and Ischnocerina, for example, Bovicola spp., Damalina spp., Felicola spp.; Lepikentron spp., Menopon spp., Trichodectes spp., Trimenopon spp., Trinoton spp., Wemeckiella spp;
from the order of Diptera and the suborders Nematocerina and Brachycerina, for example, Aedes spp., Anopheles spp., Atylotus spp., Braula spp., Calliphora spp., Chrysomyia spp., Chrysops spp., Culex spp., Culicoides spp., Eusimulium spp., Fannia spp., Gasterophilus spp., Glossina spp., Haematobia spp., Haematopota spp., Hippobosca spp., Hybomitra spp., Hydrotaea spp., Hypoderma spp., Lipoptena spp., Lucilia spp., Lutzomyia spp., Melophagus spp., Morellia spp., Musca spp., Odagmia spp., Oestrus spp., Philipomyia spp., Phlebotomus spp., Rhinoestrus spp., Sarcophaga spp., Simulium spp., Stomoxys spp., Tabanus spp., Tipula spp., Wilhelmia spp., Wohlfahrtia spp.;
from the order of Siphonapterida, for example, Ceratophyllus spp., Ctenocephalides spp., Pulex spp., Tunga spp., Xenopsylla spp.;
from the order of Heteropterida, for example, Cimex spp., Panstrongylus spp., Rhodnius spp., Triatoma spp.; and also nuisance and hygiene pests from the order Blattarida.

In addition, in the case of the arthropods, mention should be made by way of example, without limitation, of the following Acari:

from the subclass of Acari (Acarina) and the order of Metastigmata, for example from the family of Argasidae such as Argas spp., Omithodorus spp., Otobius spp., from the family of Ixodidae such as Amblyomma spp., Dermacentor spp., Haemaphysalis spp., Hyalomma spp., Ixodes spp., Rhipicephalus (Boophilus) spp., Rhipicephalus spp. (the original genus of multi-host ticks); from the order of Mesostigmata such as Dermanyssus spp., Omithonyssus spp., Pneumonyssus spp., Raillietia spp., Sternostoma spp., Tropilaelaps spp., Varroa spp.; from the order of the Actinedida (Prostigmata), for example, Acarapis spp., Cheyletiella spp., Demodex spp., Listrophorus spp., Myobia spp., Neotrombicula spp., Omithocheyletia spp., Psorergates spp., Trombicula spp.; and from the order of the Acaridida (Astigmata), for example, Acarus spp., Caloglyphus spp., Chorioptes spp., Cytodites spp., Hypodectes spp., Knemidocoptes spp., Laminosioptes spp., Notoedres spp., Otodectes spp., Psoroptes spp., Pterolichus spp., Sarcoptes spp., Trixacarus spp., Tyrophagus spp.

Examples of parasitic protozoa include, but are not limited to:

Mastigophora (Flagellata), such as:

Metamonada: from the order of Diplomonadida, for example, Giardia spp., Spironucleus spp.

Parabasala: from the order of Trichomonadida, for example, Histomonas spp., Pentatrichomonas spp., Tetratrichomonas spp., Trichomonas spp., Tritrichomonas spp.

Euglenozoa: from the order of Trypanosomatida, for example, Leishmania spp., Trypanosoma spp.

Sarcomastigophora (Rhizopoda) such as Entamoebidae, for example, Entamoeba spp., Centramoebidae, for example Acanthamoeba sp., Euamoebidae, e.g. Hartmanella sp.

Alveolata such as Apicomplexa (Sporozoa): e.g. Cryptosporidium spp.; from the order of Eimeriida, for example, Besnoitia spp., Cystoisospora spp., Eimeria spp., Hammondia spp., Isospora spp., Neospora spp., Sarcocystis spp., Toxoplasma spp.; from the order of Adeleida, for example, Hepatozoon spp., Klossiella spp.; from the order of Haemosporida, for example, Leucocytozoon spp., Plasmodium spp.; from the order of Piroplasmida, for example, Babesia spp., Ciliophora spp., Echinozoon spp., Theileria spp.; from the order of Vesibuliferida, for example, Balantidium spp., Buxtonella spp.

Microspora such as Encephalitozoon spp., Enterocytozoon spp., Globidium spp., Nosema spp., and also, for example, Myxozoa spp.

The helminths that are pathogenic to humans or animals include, for example, Acanthocephala, nematodes, Pentastoma and Platyhelminthes (e.g. Monogenea, cestodes and trematodes).

Illustrative helminths include, but are not limited to:

Monogenea: e.g. Dactylogyrus spp., Gyrodactylus spp., Microbothrium spp., Polystoma spp., Troglecephalus spp.;

Cestodes: from the order of Pseudophyllidea, for example: Bothridium spp., Diphyllobothrium spp., Diplogonoporus spp., Ichthyobothrium spp., Ligula spp., Schistocephalus spp., Spirometra spp.

From the order of Cyclophyllida, for example: Andyra spp., Anoplocephala spp., Avitellina spp., Bertiella spp., Cittotaenia spp., Davainea spp., Diorchis spp., Diplopylidium spp., Dipylidium spp., Echinococcus spp., Echinocotyle spp., Echinolepis spp., Hydatigera spp., Hymenolepis spp., Joyeuxiella spp., Mesocestoides spp., Moniezia spp., Paranoplocephala spp., Raillietina spp., Stilesia spp., Taenia spp., Thysaniezia spp., Thysanosoma spp.

Trematodes: from the class of Digenea, for example: Austrobilharzia spp., Brachylaima spp., Calicophoron spp., Catatropis spp., Clonorchis spp. Collyriclum spp., Cotylophoron spp., Cyclocoelum spp., Dicrocoelium spp., Diplostomum spp., Echinochasmus spp., Echinoparyphium spp., Echinostoma spp., Eurytrema spp., Fasciola spp., Fasciolides spp., Fasciolopsis spp., Fischoederius spp., Gastrothylacus spp., Gigantobilharzia spp., Gigantocotyle spp., Heterophyes spp., Hypoderaeum spp., Leucochloridium spp., Metagonimus spp., Metorchis spp., Nanophyetus spp., Notocotylus spp., Opisthorchis spp., Omithobilharzia spp., Paragonimus spp., Paramphistomum spp., Plagiorchis spp., Posthodiplostomum spp., Prosthogonimus spp., Schistosoma spp., Trichobilharzia spp., Troglotrema spp., Typhlocoelum spp.

Nematodes: from the order of Trichinellida, for example: Capillaria spp., Trichinella spp., Trichomosoides spp., Trichuris spp.

From the order of Tylenchida, for example: Micronema spp., Parastrangyloides spp., Strongyloides spp.

From the order of Rhabditina, for example: Aelurostrongylus spp., Amidostomum spp., Ancylostoma spp., Angiostrongylus spp., Bronchonema spp., Bunostomum spp., Chabertia spp., Cooperia spp., Cooperioides spp., Crenosoma spp., Cyathostomum spp., Cyclococercus spp., Cyclodontostomum spp., Cylicocyclus spp., Cylicostephanus spp., Cylindropharynx spp., Cystocaulus spp., Dictyocaulus spp., Elaphostrongylus spp., Filaroides spp., Globocephalus spp., Graphidium spp., Gyalocephalus spp., Haemonchus spp., Heligmosomoides spp., Hyostrongylus spp., Marshallagia spp., Metastrongylus spp., Muellerius spp., Necator spp., Nematodirus spp., Neostrongylus spp., Nippostrongylus spp., Obeliscoides spp., Oesophagodontus spp., Oesophagostomum spp., Ollulanus spp.; Omithostrongylus spp., Oslerus spp., Ostertagia spp., Paracooperia spp., Paracrenosoma spp., Parafilaroides spp., Parelaphostrongylus spp., Pneumocaulus spp., Pneumostrongylus spp., Poteriostomum spp., Protostrongylus spp., Spicocaulus spp., Stephanurus spp., Strongylus spp., Syngamus spp., Teladorsagia spp., Trichonema spp., Trichostrongylus spp., Triodontophorus spp., Troglostrongylus spp., Uncinaria spp.

From the order of Spirurida, for example: Acanthocheilonema spp., Anisakis spp., Ascaridia spp.; Ascaris spp., Ascarops spp., Aspiculuris spp., Baylisascaris spp., Brugia spp., Cercopithifilaria spp., Crassicauda spp., Dipetalonema spp., Dirofilaria spp., Dracunculus spp.; Draschia spp., Enterobius spp., Filaria spp., Gnathostoma spp., Gongylonema spp., Habronema spp., Heterakis spp.; Litomosoides spp., Loa spp., Onchocerca spp., Oxyuris spp., Parabronema spp., Parafilaria spp., Parascaris spp., Passalurus spp., Physaloptera spp., Probstmayria spp., Pseudofilaria spp., Setaria spp., Skjrabinema spp., Spirocerca spp., Stephanofilaria spp., Strongyluris spp., Syphacia spp., Thelazia spp., Toxascaris spp., Toxocara spp., Wuchereria spp.

Acanthocephala: from the order of Oligacanthorhynchida, for example: Macracanthorhynchus spp., Prosthenorchis spp.; from the order of Moniliformida, for example: Moniliformis spp.

From the order of Polymorphida, for example: Filicollis spp.; from the order of Echinorhynchida, for example Acanthocephalus spp., Echinorhynchus spp., Leptorhynchoides spp.

Pentastoma: from the order of Porocephalida, for example, Linguatula spp.

In the veterinary field and in animal husbandry, the compounds of the formula (I) are administered by methods generally known in the art, such as via the enteral, parenteral, dermal or nasal route in the form of suitable preparations. Administration may be prophylactic, metaphylactic or therapeutic.

Thus, one embodiment of the present invention refers to the compounds of the formula (I) for use as a medicament.

A further aspect relates to the compounds of the formula (I) for use as an antiendoparasitic agent.

A further specific aspect of the invention relates to the compounds of the formula (I) for use as an antithelminthic agent, especially for use as a nematicide, platyhelminthicide, acanthocephalicide or pentastomicide.

A further specific aspect of the invention relates to the compounds of the formula (I) for use as an antiprotozoic agent.

A further aspect relates to the compounds of the formula (I) for use as an antiectoparasitic agent, especially an arthropodicide, very particularly an insecticide or an acaricide.

Further aspects of the invention are veterinary medicine formulations comprising an effective amount of at least one compound of the formula (I) and at least one of the following: a pharmaceutically acceptable excipient (e.g. solid or liquid diluents), a pharmaceutically acceptable auxiliary (e.g. surfactants), especially a pharmaceutically acceptable excipient used conventionally in veterinary medicine formulations and/or a pharmaceutically acceptable auxiliary conventionally used in veterinary medicine formulations.

A related aspect of the invention is a method for production of a veterinary medicine formulation as described here, which comprises the step of mixing at least one compound of the formula (I) with pharmaceutically acceptable excipients and/or auxiliaries, especially with pharmaceutically acceptable excipients used conventionally in veterinary medicine formulations and/or auxiliaries used conventionally in veterinary medicine formulations.

Another specific aspect of the invention is veterinary medicine formulations selected from the group of ectoparasiticidal and endoparasiticidal formulations, especially selected from the group of anthelmintic, antiprotozoic and arthropodicidal formulations, very particularly selected from the group of nematicidal, platyhelminthicidal, acanthocephalicidal, pentastomicidal, insecticidal and acaricidal formulations, according to the aspects mentioned, and methods for production thereof.

Another aspect relates to a method for treatment of a parasitic infection, especially an infection caused by a parasite selected from the group of the ectoparasites and endoparasites mentioned here, by use of an effective amount of a compound of the formula (I) in an animal, especially a nonhuman animal, having a need therefor.

Another aspect relates to a method for treatment of a parasitic infection, especially an infection caused by a parasite selected from the group of the ectoparasites and endoparasites mentioned here, by use of a veterinary medicine formulation as defined here in an animal, especially a nonhuman animal, having a need therefor.

Another aspect relates to the use of the compounds of the formula (I) in the treatment of a parasite infection, especially an infection caused by a parasite selected from the group of the ectoparasites and endoparasites mentioned here, in an animal, especially a nonhuman animal.

In the present context of animal health or veterinary medicine, the term “treatment” includes prophylactic, metaphylactic and therapeutic treatment.

In a particular embodiment, in this way, mixtures of at least one compound of the formula (I) with other active ingredients, especially with endo- and ectoparasiticides, are provided for the field of veterinary medicine.

In the field of animal health, “mixture” means not just that two (or more) different active ingredients are formulated in a common formulation and are correspondingly employed together, but also relates to products comprising formulations separated for each active ingredient. Accordingly, when more than two active ingredients are to be employed, all active ingredients can be formulated in a common formulation or all active ingredients can be formulated in separate formulations; likewise conceivable are mixed forms in which some of the active ingredients are formulated together and some of the active ingredients are formulated separately. Separate formulations allow the separate or successive application of the active ingredients in question.

The active ingredients specified here by their “common names” are known and are described, for example, in the “Pesticide Manual” (see above) or can be searched for on the Internet (e.g.: http://www.alanwood.net/pesticides).

Illustrative active ingredients from the group of the ectoparasiticides as mixing components, without any intention that this should constitute a restriction, include the insecticides and acaricides listed in detail above. Further usable active ingredients are listed below in accordance with the abovementioned classification based on the current IRAC Mode of Action Classification Scheme: (1) acetylcholinesterase (AChE) inhibitors; (2) GABA-gated chloride channel blockers; (3) sodium channel modulators; (4) nicotinic acetylcholine receptor (nAChR) competitive modulators; (5) nicotinic acetylcholine receptor (nAChR) allosteric modulators; (6) glutamate-gated chloride channel (GluCl) allosteric modulators; (7) juvenile hormone mimetics; (8) miscellaneous non-specific (multi-site) inhibitors; (9) chordotonal organ modulators; (10) mite growth inhibitors; (12) inhibitors of mitochondrial ATP synthase, such as ATP disruptors; (13) uncouplers of oxidative phosphorylation via disruption of the proton gradient; (14) nicotinic acetylcholine receptor channel blockers; (15) inhibitors of chitin biosynthesis, type 0; (16) inhibitors of chitin biosynthesis, type 1; (17) moulting disruptors (especially in Diptera); (18) ecdysone receptor agonists; (19) octopamine receptor agonists; (21) mitochondrial complex I electron transport inhibitors; (25) mitochondrial complex II electron transport inhibitors; (20) mitochondrial complex III electron transport inhibitors; (22) voltage-dependent sodium channel blockers; (23) inhibitors of acetyl CoA carboxylase; (28) ryanodine receptor modulators;

active ingredients having unknown or non-specific mechanisms of action, e.g. fentrifanil, fenoxacrim, cycloprene, chlorobenzilate, chlordimeform, flubenzimin, dicyclanil, amidoflumet, quinomethionat, triarathene, clothiazoben, tetrasul, potassium oleate, petroleum, metoxadiazone, gossyplur, flutenzine, brompropylate, cryolite;
compounds from other classes, for example butacarb, dimetilan, cloethocarb, phosphocarb, pirimiphos(-ethyl), parathion(-ethyl), methacrifos, isopropyl o-salicylate, trichlorfon, sulprofos, propaphos, sebufos, pyridathion, prothoate, dichlofenthion, demeton-S-methyl sulphone, isazofos, cyanofenphos, dialifos, carbophenothion, autathiofos, aromfenvinfos(-methyl), azinphos(-ethyl), chlorpyrifos(-ethyl), fosmethilan, iodofenphos, dioxabenzofos, formothion, fonofos, flupyrazofos, fensulfothion, etrimfos;
organochlorine compounds, for example camphechlor, lindane, heptachlor; or phenylpyrazoles, e.g. acetoprole, pyrafluprole, pyriprole, vaniliprole, sisapronil; or isoxazolines, e.g. sarolaner, afoxolaner, lotilaner, fluralaner;
pyrethroids, e.g. (cis-, trans-)metofluthrin, profluthrin, flufenprox, flubrocythrinate, fubfenprox, fenfluthrin, protrifenbut, pyresmethrin, RU15525, terallethrin, cis-resmethrin, heptafluthrin, bioethanomethrin, biopermethrin, fenpyrithrin, cis-cypermethrin, cis-permethrin, clocythrin, cyhalothrin (lambda-), chlovaporthrin, or halogenated hydrocarbon compounds (HCHs),
neonicotinoids, e.g. nithiazine
dicloromezotiaz, triflumezopyrim
macrocyclic lactones, e.g. nemadectin, ivermectin, latidectin, moxidectin, selamectin, eprinomectin, doramectin, emamectin benzoate; milbemycin oxime
triprene, epofenonane, diofenolan;
biologicals, hormones or pheromones, for example natural products, e.g. thuringiensin, codlemone or neem components
dinitrophenols, e.g. dinocap, dinobuton, binapacryl;
benzoylureas, e.g. fluazuron, penfluron,
amidine derivatives, e.g. chlormebuform, cymiazole, demiditraz
beehive varroa acaricides, for example organic acids, e.g. formic acid, oxalic acid.

Illustrative active ingredients from the group of the endoparasiticides, as mixing components, include, but are not limited to, active anthelmintic ingredients and active antiprotozoic ingredients.

The active anthelmintic ingredients include but are not limited to the following active nematicidal, trematicidal and/or cestocidal ingredients:

from the class of the macrocyclic lactones, for example: eprinomectin, abamectin, nemadectin, moxidectin, doramectin, selamectin, lepimectin, latidectin, milbemectin, ivermectin, emamectin, milbemycin;
from the class of the benzimidazoles and probenzimidazoles, for example: oxibendazole, mebendazole, triclabendazole, thiophanate, parbendazole, oxfendazole, netobimin, fenbendazole, febantel, thiabendazole, cyclobendazole, cambendazole, albendazole sulphoxide, albendazole, flubendazole;
from the class of the depsipeptides, preferably cyclic depsipeptides, especially 24-membered cyclic depsipeptides, for example: emodepside, PF1022A;
from the class of the tetrahydropyrimidines, for example: morantel, pyrantel, oxantel;
from the class of the imidazothiazoles, for example: butamisole, levamisole, tetramisole;
from the class of the aminophenylamidines, for example: amidantel, deacylated amidantel (dAMD), tribendimidine;
from the class of the aminoacetonitriles, for example: monepantel;
from the class of the paraherquamides, for example: paraherquamide, derquantel;
from the class of the salicylanilides, for example: tribromsalan, bromoxanide, brotianide, clioxanide, closantel, niclosamide, oxyclozanide, rafoxanide;
from the class of the substituted phenols, for example: nitroxynil, bithionol, disophenol, hexachlorophen, niclofolan, meniclopholan;
from the class of the organophosphates, for example: trichlorfon, naphthalofos, dichlorvos/DDVP, crufomate, coumaphos, haloxon;
from the class of the piperazinones/quinolines, for example: praziquantel, epsiprantel;
from the class of the piperazines, for example: piperazine, hydroxyzine;
from the class of the tetracyclines, for example: tetracycline, chlorotetracycline, doxycycline, oxytetracycline, rolitetracycline;
from various other classes, for example: bunamidine, niridazole, resorantel, omphalotin, oltipraz, nitroscanate, nitroxynil, oxamniquin, mirasan, miracil, lucanthon, hycanthon, hetolin, emetin, diethylcarbamazine, dichlorophen, diamfenetide, clonazepam, bephenium, amoscanate, clorsulon.

Active antiprotozoic ingredients include, but are not limited to, the following active ingredients:

from the class of the triazines, for example: diclazuril, ponazuril, letrazuril, toltrazuril;
from the class of polyether ionophores, for example: monensin, salinomycin, maduramicin, narasin;
from the class of the macrocyclic lactones, for example: milbemycin, erythromycin;
from the class of the quinolones, for example: enrofloxacin, pradofloxacin;
from the class of the quinines, for example: chloroquin;
from the class of the pyrimidines, for example: pyrimethamine;
from the class of the sulphonamides, for example: sulfaquinoxaline, trimethoprim, sulfaclozin;
from the class of the thiamines, for example: amprolium;
from the class of the lincosamides, for example: clindamycin;
from the class of the carbanilides, for example: imidocarb;
from the class of the nitrofurans, for example: nifurtimox;
from the class of the quinazolinone alkaloids, for example: halofuginone;
from various other classes, for example: oxamniquin, paromomycin;
from the class of the vaccines or antigens from microorganisms, for example: Babesia canis rossi, Eimeria tenella, Eimeria praecox, Eimeria necatrix, Eimeria mitis, Eimeria maxima, Eimeria brunetti, Eimeria acervulina, Babesia canis vogeli, Leishmania infantum, Babesia canis canis, Dictyocaulus viviparus.

All the mixing components mentioned, as the case may be, may also form salts with suitable bases or acids if they are capable of doing so on the basis of their functional groups.

### Vector Control

The compounds of the formula (I) can also be used in vector control. In the context of the present invention, a vector is an arthropod, especially an insect or arachnid, capable of transmitting pathogens, for example viruses, worms, single-cell organisms and bacteria, from a reservoir (plant, animal, human, etc.) to a host. The pathogens can be transmitted either mechanically (for example trachoma by non-stinging flies) onto a host or after injection into a host (for example malaria parasites by mosquitoes).

Examples of vectors and the diseases or pathogens they transmit are:

1) mosquitoes
Anopheles: malaria, filariasis;
Culex: Japanese encephalitis, filariasis, other viral diseases, transmission of other worms;
Aedes: yellow fever, dengue fever, further viral disorders, filariasis;
Simuliidae: transmission of worms, especially Onchocerca volvulus;
Psychodidae: transmission of leishmaniasis
   2) Lice: skin infections, epidemic typhus;
   3) Fleas: plague, endemic typhus, tapeworms;
   4) Flies: sleeping sickness (trypanosomiasis); cholera, other bacterial diseases;
   5) Mites: acariosis, epidemic typhus, rickettsialpox, tularaemia, Saint Louis encephalitis, tick-bome encephalitis (TBE), Crimean-Congo haemorrhagic fever, borreliosis;
   6) Ticks: borellioses such as Borrelia bungdorferi sensu lato., Borrelia duttoni, tick-borne encephalitis, Q fever (Coxiella burnetii), babesioses (Babesia canis canis), ehrlichiosis.

Examples of vectors in the context of the present invention are insects, for example aphids, flies, leafhoppers or thrips, which can transmit plant viruses to plants. Other vectors capable of transmitting plant viruses are spider mites, lice, beetles and nematodes.

Further examples of vectors in the context of the present invention are insects and arachnids such as mosquitoes, especially of the genera Aedes, Anopheles, for example A. gambiae, A. arabiensis, A. funestus, A. dirus (malaria) and Culex, Psychodidae such as Phlebotomus, Lutzomyia, lice, fleas, flies, mites and ticks, which can transmit pathogens to animals and/or humans.

Vector control is also possible if the compounds of the formula (I) are resistance-breaking.

Compounds of the formula (I) are suitable for use in the prevention of diseases and/or pathogens transmitted by vectors. Thus, a further aspect of the present invention is the use of compounds of the formula (I) for vector control, for example in agriculture, in horticulture, in forests, in gardens and in leisure facilities, and also in the protection of materials and stored products.

### Protection of Industrial Materials

The compounds of the formula (I) are suitable for protecting industrial materials against attack or destruction by insects, for example from the orders of Coleoptera, Hymenoptera, Isoptera, Lepidoptera, Psocoptera and Zygentoma.

Industrial materials in the present context are understood to mean inanimate materials, such as preferably plastics, adhesives, sizes, papers and cards, leather, wood, processed wood products and coating compositions. The use of the invention for protection of wood is particularly preferred.

In a further embodiment, the compounds of the formula (I) are used together with at least one further insecticide and/or at least one fungicide.

In a further embodiment, the compounds of the formula (I) take the form of a ready-to-use pesticide, meaning that they can be applied to the material in question without further modifications. Useful further insecticides or fungicides especially include those mentioned above.

Surprisingly, it has also been found that the compounds of the formula (I) can be employed for protecting objects which come into contact with saltwater or brackish water, in particular hulls, screens, nets, buildings, moorings and signalling systems, against fouling. It is equally possible to use the compounds of the formula (I), alone or in combinations with other active ingredients, as antifouling agents.

### Control of Animal Pests in the Hygiene Sector

The compounds of the formula (I) are suitable for controlling animal pests in the hygiene sector. More particularly, the invention can be used in the domestic protection sector, in the hygiene protection sector and in the protection of stored products, particularly for control of insects, arachnids, ticks and mites encountered in enclosed spaces, for example dwellings, factory halls, offices, vehicle cabins, animal breeding facilities. For controlling animal pests, the compounds of the formula (I) are used alone or in combination with other active ingredients and/or auxiliaries. They are preferably used in domestic insecticide products. The compounds of the formula (I) are effective against sensitive and resistant species, and against all developmental stages.

These pests include, for example, pests from the class Arachnida, from the orders Scorpiones, Araneae and Opiliones, from the classes Chilopoda and Diplopoda, from the class Insecta the order Blattodea, from the orders Coleoptera, Dermaptera, Diptera, Heteroptera, Hymenoptera, Isoptera, Lepidoptera, Phthiraptera, Psocoptera, Saltatoria or Orthoptera, Siphonaptera and Zygentoma and from the class Malacostraca the order Isopoda.

Application is effected, for example, in aerosols, unpressurized spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or bait stations.

### Elucidation of the Processes and Intermediates:

The inventive compounds of the formula (I) can be obtained by the processes shown in the following schemes:

### Process A

The inventive compounds of the formula (I-c) shown in Process A, in which V¹ and V² are oxygen, can be prepared by the sequence of methods known from the literature according to the following scheme:

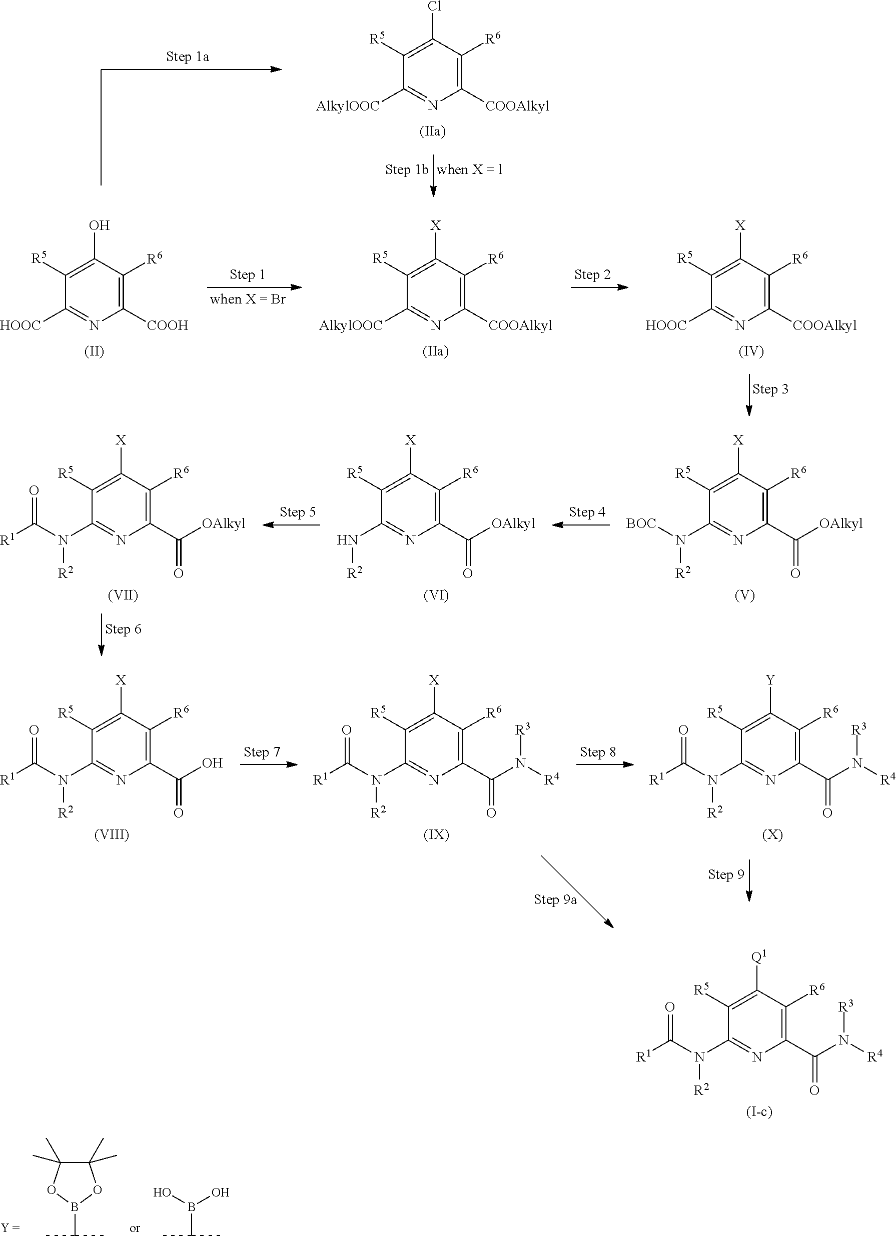

The R¹, R³, R⁴, R⁵, R⁶ and Q¹ radicals have the definitions described above, where Q¹ is C—C-bonded to the rest of the molecule. X—unless further restricted in the scheme—is halogen, especially chlorine, bromine or iodine. R² is hydrogen. BOC=tert-butyloxycarbonyl.

The invention also relates to the intermediates of the general formulae (VII), (VIII), (IX) and (X) where R¹, R³, R⁴, R⁵ and R⁶ have the definitions described above, R² is hydrogen, X is halogen and Y is

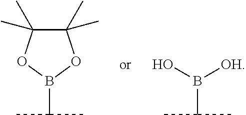

### Step 1:

4-Hydroxypyridine-2,6-dicarboxylic acids of the general formula (II) are suitable as starting materials for the preparation of the dialkyl esters of the formula (III) by methods known from the literature. Some of them are commercially available, for example 4-hydroxypyridine-2,6-dicarboxylic acid (R⁵=R⁶=hydrogen), or can be prepared by known methods from commercially available synthons, for example from corresponding substituted 4-chloropyridines for 3,5-dichloro-4-hydroxypyridine-2,6-dicarboxylic acid (R⁵=R⁶=chlorine) by the synthesis described in U.S. Pat. No. 3,651,070.

The hydroxyl function of the 4-hydroxypyridine-2,6-dicarboxylic acids (II) can, for example, in analogy to the processes described in U.S. Pat. No. 6,355,653 or US2004/29851, be converted to the corresponding bromine substituents in the presence of phosphorus pentabromide in suitable inert solvents, for example carbon tetrachloride or chlorobenzene. By quenching of the reaction mixture with an alcohol, for example methanol in the case of the process according to US2004/29851, the dialkyl ester of the formula (III) is produced here directly.

### Step 1a:

The hydroxyl function of the 4-hydroxypyridine-2,6-dicarboxylic acids (II) can be converted to the corresponding chlorine substituent in a similar manner to Step 1. A suitable process for the reaction with thionyl chloride in a suitable inert solvent, for example dimethylformamide, can be found, for example, in US2014/336373. As an alternative, Step 1a can also be converted to the corresponding dialkyl ester of the formula (IIa) by reaction of (II) with phosphorus pentachloride in a suitable inert solvent, for example carbon tetrachloride, and subsequent reaction with an alcohol, e.g. methanol (cf, in analogy, for example, Tetrahedron, 2005, 61, 1755-1763).

### Step 1b:

Conversion of the chlorine substituent of the compound (IIa) to iodine, resulting in the corresponding compound of the formula (III) with X═I, proceeds by methods known from the literature (cf., in analogy, for example, Tetrahedron 2005, 61, 1755-1763), for example by reaction of (IIa) with sodium iodide in suitable inert solvents, e.g. acetonitrile.

Some 2,6-dialkyl-4-halopyridine-2,6-dicarboxylates of the formula (III) are known from the literature, and they can also be prepared by known methods from 4-amino- or 4-halopyridines. Examples known from the literature include diethyl 3,5-dichloro-4-iodopyridine-2,6-dicarboxylate (X=iodine, alkyl=ethyl, R⁵=R⁶=chlorine) in DE579225 and dimethyl 3,4,5-trichloropyridine-2,6-dicarboxylate (X=chlorine, alkyl=methyl, R⁵=R⁶=chlorine) in U.S. Pat. No. 3,637,716. For example, dimethyl 4-amino-3-cyanopyridine-2,6-dicarboxylate (alkyl=methyl, R⁵=cyano, R⁶=hydrogen) is suitable for the preparation of various dimethyl 3-cyano-4-halopyridine-2,6-dicarboxylates (X=halogen, alkyl=methyl, R⁵=cyano, R⁶=hydrogen), for example according to ACS Med. Chem. Lett. 2014, 5(8), 921-926, WO2007/093901 or Tet. Let. 2011, 52(44), 5728-5732.

### Step 2:

Dialkyl esters of the formula (III) can be converted to the corresponding monoalkyl esters of the formula (IV) by hydrolysis with the aid of a base, for example potassium hydroxide, in suitable inert solvents or solvent mixtures, for example methanol/dichloromethane 10:1 (cf. EP 2 017 279).

### Step 3:

The acid function of the monoalkyl esters obtained in Step 2 can also be converted to a t-butoxycarbonyl-protected (BOC-protected) amino group by methods known from the literature, for example according to EP 2 017 279, by Curtius rearrangement, for example by reaction with diphenylphosphoryl azide in suitable inert solvents, for example dioxane, and in the presence of t-butanol and triethylamine, giving compounds of the formula (V).

### Step 4:

For conversion of the compounds of the formula (V) to the analogous compounds of the formula (VI), the BOC protecting group can be detached by reaction of (V) with an acid, for example trifluoroacetic acid, in a suitable inert solvent, for example chloroform or dichloromethane (cf. EP 2 017 279).

### Step 5:

The acylation of the compounds of the formula (VI) to give compounds of the formula (VII) proceeds by methods known from the literature, for example by the reaction of (VI) with a suitable acid chloride in the presence of suitable bases, for example pyridine, triethylamine or potassium carbonate, in a suitable inert solvent, for example dichloromethane or 2-butanone. Analogous processes are described, for example, in EP1714966, U.S. Pat. No. 5,403,816 or WO2004/35545.

Alternatively, compounds of the formula (VII) can also be obtained by reaction of (VI) with carboxylic acids in the presence of suitable coupling reagents, for example HATU (O-(7-azabenzotriazol-1-yl)-N,N,N′-tetramethyluronium hexafluorophosphate]) or EDCI (1-ethyl-3-(3-dimethylaminopropyl)carbodiimide), and suitable bases, for example triethylamine or N,N-diisopropylethylamine, in suitable inert solvents, for example DMF (dimethylformamide) or dichloromethane (cf, in analogy, for example, US2011/301181 or WO2007/122258).

The carboxylic acids are either commercially available or can be prepared by known methods.

### Step 6:

The cleavage of the ester functions of the formula (VII) for preparation of (VIII) proceeds by methods known from the literature (cf., in analogy, for example, US2007/213349, WO2015/150440 or US2011/301181), by reaction with a suitable base, for example lithium hydroxide or sodium hydroxide, in suitable aqueous solvent mixtures, e.g. water/THF, water/methanol or water/dioxane, or water.

### Step 7:

The resultant carboxylic acids of the formula (VIII) can be reacted with primary or secondary amines, alkoxyamines or hydrazines, in the presence of suitable coupling reagents, e.g. HATU or EDCI, and of suitable bases, e.g. triethylamine or N,N-diisopropylethylamine, in suitable inert solvents, for example DMF or dichloromethane, to give the corresponding amides (IX) (cf., in analogy, for example, US2011/301181 or WO2007/122258).

Alternatively, conversion of pyridine-2-carboxylic acids to (IX) can also be effected by methods known from the literature (cf., in analogy, for example, Bioorganic and Medicinal Chemistry Letters, 2006, Vol. 16, #10, pages 2689-2692) by activation to give the acid chloride, for example by reaction with oxalyl chloride or thionyl chloride, in an inert solvent, for example dichloromethane (optionally with catalytic amounts of DMF), and subsequent reaction with the appropriate amine in the presence of a base, for example triethylamine or N,N-diisopropylethylamine, in a suitable inert solvent, for example dichloromethane.

The amines, alkoxyamines and hydrazines are either commercially available or can be prepared by known methods.

### Step 8:

To obtain the corresponding boronic acids or dioxoborolane derivatives (X) from the halogenated compounds (IX), it is likewise possible to use methods known from the literature. For example, 4-bromopyridine-2-carboxamides can be converted to the corresponding boronic acid or dioxoborolane compounds by reaction with bis(pinacolato)diboron [4,4,4′,4′,5,5,5′,5′-octamethyl-2,2′-bis-1,3,2-dioxaborolane] in the presence of potassium acetate and suitable catalysts, for example dichloro(1,1′-bis(diphenylphosphanyl)ferrocene)palladium(II) [optionally as the dichloromethane adduct] or dichlorobis(triphenylphosphine)palladium(II), in suitable inert solvents, e.g. dimethylformamide, dioxane or toluene and under an inert atmosphere (cf., in analogy, for example US2004/67982, US2007/197553 or WO2011/159554).

In order to obtain compounds of the formula (IX) or (X), Step 5 can also be exchanged in the order with the sequence of Steps 6 & 7.

### Step 9:

Dioxoborolanepyridines or pyridineboronic acids of the formula (X) can be coupled to aryl or hetaryl halides, especially chlorides, bromides or iodides, or with aryl or hetaryl triflates, by known methods to give aryl- or hetarylpyridines. This reaction can be effected in a suitable solvent, for example dimethylformamide, or a suitable solvent mixture, for example dimethylformamide/water, 1,4-dioxane/water or isopropanol/toluene/water, by palladium catalysis, for example with tetrakis(triphenylphosphine)palladium(0) [cf, or example, WO2014/170821], tris(dibenzylideneacetone)dipalladium(0) [cf, for example, WO2013/61081] or (1,1′-bis(diphenylphosphino)ferrocene)palladium(II) dichloride [cf, for example, US2011/9410] as the palladium source and in the presence of a suitable base, for example potassium carbonate or sodium carbonate. In this way, it is also possible to obtain compounds of the formula (I-c).

### Step 9a:

Halogenated pyridines of the formula (IX) can be coupled to aryl- and heteroarylboronic acids or aryl- and heteroarylboronic esters by known methods to give aryl- or hetarylpyridines. This reaction can be effected in a suitable solvent, for example dimethylformamide, or a suitable solvent mixture, for example dimethylformamide/water, 1,4-dioxane/water or isopropanol/toluene/water, by palladium catalysis, for example with tetrakis(triphenylphosphine)palladium(0) [cf, for example, US2015/210671], tris(dibenzylideneacetone)dipalladium(0) [cf, for example, WO2013/61081] or (1,1′-bis(diphenylphosphino)ferrocene)palladium(II) dichloride [cf, for example, US2011/9410] as the palladium source and in the presence of a suitable base, for example potassium carbonate or sodium carbonate. In this way, it is also possible to obtain compounds of the formula (I-c).

### Process B

The inventive compounds of the formula (I-c) shown in Process B, in which V¹ and V² are oxygen, can be prepared by the sequence of methods known from the literature according to the following scheme:

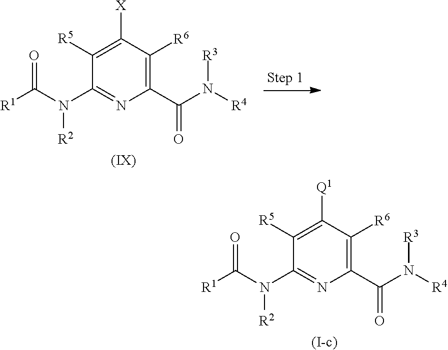

The R¹, R³, R⁴, R⁵, R⁶ and Q¹ radicals have the definitions described above, where Q¹ is C—N-bonded to the rest of the molecule. X is halogen, preferably bromine or iodine. R² is hydrogen.

### Step 1:

The halogenated pyridines of the formula (IX) can be coupled to NH-containing heteroaromatics, for example pyrroles, imidazoles or pyrazoles, for example, in analogy to US2013/165464 or WO2012/64815 under an inert gas atmosphere by catalysis with copper(I) salts, for example copper(I) iodide, in the presence of a suitable ligand, for example (trans)-N,N′-dimethylcyclohexane-1,2-diamine or R-(+)-proline, and a suitable base, for example potassium carbonate, in a suitable solvent, for example 1,4-dioxane or dimethyl sulphoxide, giving compounds of the formula (I-c).

### Process C

The inventive compounds of the formula (I-c) shown in Process C, in which V¹ and V² are oxygen, can be prepared by the sequence of methods known from the literature according to the following scheme:

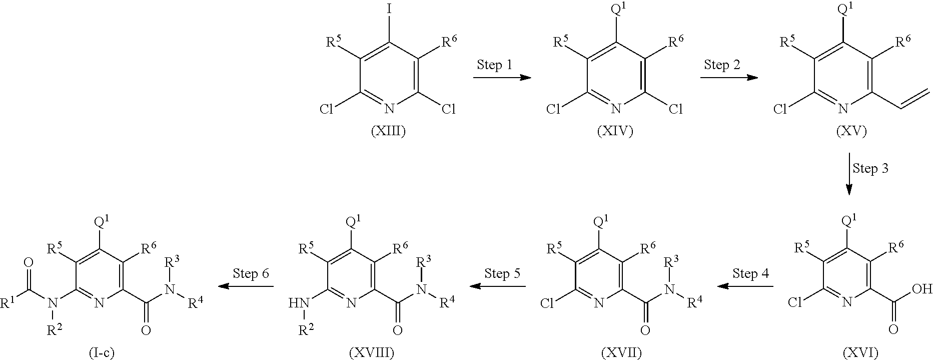

The R¹, R², R³, R⁴, R⁵, R⁶ and Q¹ radicals have the definitions described above.

The invention also relates to the intermediates of the general formulae (XVI), (XVII) and (XVIII) where R², R³, R⁴, R⁵, R⁶ and Q¹ have the definitions described above, where Q¹ is not unsubstituted phenyl and not substituted or unsubstituted pyrazole.

### Step 1:

The 2,6-dichloro-4-iodopyridines of the formula (XIII) can be coupled at the iodine position to aromatic or heteroaromatic boronic acids, boronic esters or bisoxoborolanes to give compounds of the formula (XIV) by the known methods described in Process A, Step 9a, using suitable catalysts, for example tetrakis(triphenylphosphine)palladium(0).

In addition, compounds of the formula (XIV) can be prepared by coupling of the 2,6-dichloro-4-iodopyridines at the iodine position to NH-heterocycles in analogy to the known methods described in Process B, Step 1 [cf., for example, US2013/165464], where the bond is via the nitrogen of the heterocycle.

Some 2,6-dichloro-4-iodopyridines of the general formula (XIII) are commercially available, for example 2,6-dichloro-4-iodopyridine (R⁵=R⁶=hydrogen), 2,3,5,6-tetrachloro-4-iodopyridine (R⁵=R⁶=chlorine) or known from the literature or can be prepared by known methods. For example, 2,6-dichloro-5-fluoro-4-iodonicotinonitrile (R⁵=fluorine, R⁶=cyano) can be synthesized by the method described in WO2006/082392. 2,6-Dichloro-4-iodo-3-methylpyridine (R⁵=methyl, R⁶=hydrogen) can be prepared, for example, from 2,6-dichloro-3-methylpyridine by the protocol in J. Med. Chem. 2011, 54(19), 6691-6703.

### Step 2:

The vinylation of the compounds of the formula (XIV) to give (XV) is performable by the reaction thereof with, for example 4,4,5,5-tetramethyl-2-vinyl-1,3,2-dioxaborolane or trivinylboroxine, in analogy to the known palladium-catalysed methods described in Process A, Step 9a [cf., in addition, for example, WO2011/54773 or WO2009/24905]. Also known from the literature is use of tributylvinyltin as vinyl donor [cf, for example, Journal of Heterocyclic Chemistry, 2001, Vol. 38, #5, pages 1039-1044].

### Step 3:

The vinyl function of the compounds of the formula (XV) obtained in Step 2 can be converted to the corresponding carboxylic acid function by processes known from the literature, for example according to WO2009/24905, by means of suitable oxidizing agents, e.g. potassium permanganate, in suitable solvents or solvent mixtures, e.g. water/acetone, giving compounds of the formula (XVI).

### Step 4:

The resultant carboxylic acids of the formula (XVI) can be transformed to the corresponding amides of the formula (XVII), for example, in analogy to the methods described in Process A, Step 7, by means of coupling reagents or after activation, for example to give the carbonyl chloride.

### Step 5:

To obtain compounds of the formula (XVIII) from the precursors (XVII), it is likewise possible to use methods known from the literature. For example, according to WO2009/147190, primary amines are introduced at the chlorine position into 2-chloropyridines by direct reaction of the two coreactants while heating in a suitable solvent, for example water or dimethyl sulphoxide, optionally in the presence of a further base, for example N,N-diisopropylethylamine, and optionally under elevated pressure conditions.

In addition, compounds of the formula (XVIII) with R²=H can be obtained by reaction of (XVII) with ammonia by processes known from the literature. This reaction can be effected with aqueous ammonia [cf., for example, US2007/66644], or with aqueous ammonia in a suitable solvent, for example isopropanol (cf., for example, WO2011/110575], optionally in the presence of copper salts, e.g. copper(II) sulphate [cf., for example, US2009/162453], and optionally under elevated pressure.

Alternatively, compounds of the formula (XVIII) can be obtained by reaction of (XVII) with primary amides or primary carbamates by processes known from the literature. This reaction can be effected in a suitable solvent, for example 1,4-dioxane, by catalysis with tris(dibenzylideneacetone)dipalladium(0) [cf, for example, US2013/165464] or palladium(II) diacetate [cf., for example, WO2011/137342 or WO2014/114185] in the presence of a phosphine ligand, e.g. Xantphos (4,5-bis(diphenylphosphino)-9,9-dimethylxanthene) or Xphos (2-dicyclohexylphosphino-2′,4′,6′-triisopropylbiphenyl), and a suitable base, e.g. caesium carbonate.

### Step 6:

The preparation of the compounds of the formula (I-c) proceeds in analogy to the methods described in Process A, Step 5, by reaction of (XVIII) with a suitable acid chloride in the presence of suitable bases, for example pyridine, triethylamine or potassium carbonate, in a suitable inert solvent, for example dichloromethane.

Alternatively, compounds of the formula (I-c) can also be obtained by reaction of (XVIII) with carboxylic acids in the presence of suitable coupling reagents, for example HATU or EDCI, and suitable bases, for example triethylamine or N,N-diisopropylethylamine, in suitable inert solvents, for example DMF or dichloromethane (cf., in analogy, for example, US2011/301181 or WO2007/122258).

### Process D

The inventive compounds of the formula (I-c) shown in Process D, in which V¹ and V² are oxygen, can be prepared by the sequence of methods known from the literature according to the following scheme:

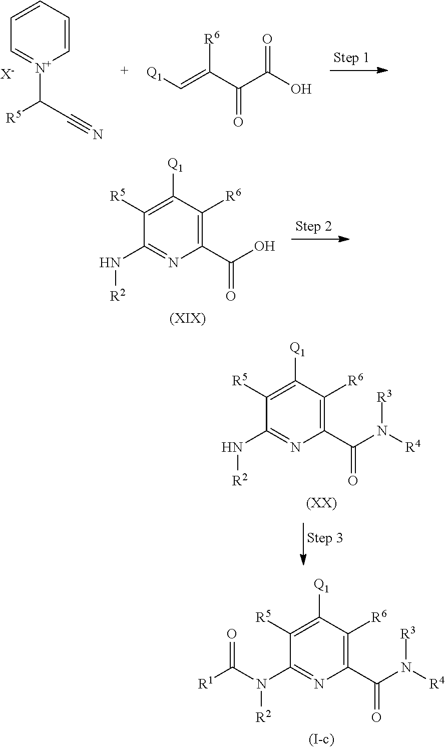

The R¹, R³, R⁴, R⁵, R⁶ and Q¹ radicals have the definitions described above, where Q¹ is C—C-bonded to the rest of the molecule. X is halogen or trifluoromethanesulphonate. R² is hydrogen.

The invention also relates to the intermediates of the general formulae (XIX) and (XX) where R² is hydrogen and R³, R⁴, R⁵, R⁶ and Q¹ have the definitions described above, where Q¹ is C—C-bonded to the rest of the molecule and is not unsubstituted phenyl.

### Step 1

The pyridines of the formula (XIX) can be prepared, for example, via a Michael reaction of a 1-(cyanomethyl)pyridinium halide and an α,β-unsaturated ketone in the presence of ammonium acetate by what is called the Krohnke pyridine synthesis, as described in Synthesis 1976, 1-24 and Angew. Chem. 1962 74, 811-817. The 6-amino-3,5-dicyano-4-phenylpyridine-2-carboxylic acid of the formula (XIX) which is known from the literature (R²=hydrogen; R⁵=R⁶=cyano; Q¹=phenyl) is described in Synth. Comm. 1992, 22(14), 2053-2056.

The starting materials for the Krohnke synthesis are commercially available or can easily be prepared. 1-(Cyanomethyl)pyridinium halides are known in the literature; for example, 1-cyanomethylpyridinium iodide (X=iodine, R⁵=R⁶=hydrogen) can be prepared by reaction of pyridine, chloroacetonitrile and potassium iodide, as described in Helv. Chim. Act. 1996 79, 1192-1202; 1-(1-cyanoethyl)pyridinium trifluoromethanesulphonate (X=trifluoromethanesulphonate (triflate), R⁵=R⁶=hydrogen) can be prepared from cyanohydrin triflate, according to Eur. J. Org. Chem. 2012, 24, 4555-4564; the synthesis of 1-(dicyanomethyl)pyridinium salts is described in Chem. Ber. 1963, 96(11), 3044-3049 and J. Am. Chem. Soc. 1965, 87/16), 3651-3656.

The α,β-unsaturated ketones required can be prepared by a number of known methods; for example, 2-oxo-4-arylbut-3-enoic acids (R⁶=hydrogen) can be isolated with the aid of sodium hydroxide solution from the corresponding aldehydes by condensation with ethyl pyruvate in a basic medium, as described, for example, in US2006/0020010; for the synthesis of 3-alkyl-2-oxo-4-arylbut-3-enoic acids (R⁶=alkyl), it is possible to react 2-oxoalkanoic acids (e.g. 2-oxobutanoic acid when R⁶=methyl) with aromatic aldehydes, as described in Tet. 1991, 47(43), 9019-9034.

### Step 2

The resultant carboxylic acids of the formula (XIX) can be transformed to the corresponding amides of the formula (XX), for example, in analogy to the methods described in Process A, Step 7, by means of coupling reagents or after activation, for example to give the carbonyl chloride.

### Step 3

The preparation of the compounds of the formula (I-c) proceeds in analogy to the methods described in Process A, Step 5, by reaction of (XX) with a suitable acid chloride in the presence of suitable bases, for example pyridine, triethylamine or potassium carbonate, in a suitable inert solvent, for example dichloromethane.

Alternatively, compounds of the formula (I-c) can also be obtained by reaction of (XX) with carboxylic acids in the presence of suitable coupling reagents, for example HATU or EDCI, and suitable bases, for example triethylamine or N,N-diisopropylethylamine, in suitable inert solvents, for example DMF or dichloromethane (cf., in analogy, for example, US2011/301181 or WO2007/122258).

In order to obtain compounds of the formula (I-c), Step 2 can alternatively be exchanged with Step 3 in the sequence.

### Process E

The inventive compounds of the formula (I-c) shown in Process E, in which V¹ and V² are oxygen, can be prepared by methods known from the literature according to the following scheme:

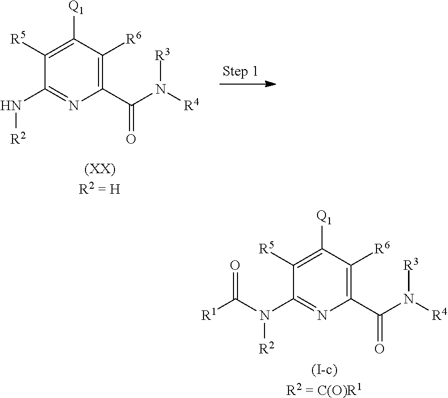

The R¹, R³, R⁴ and Q¹ radicals have the definitions described above. R⁵ and R⁶ are hydrogen. R² has the definition given in the scheme.

### Step 1

The preparation of the compounds of the formula (I-c) proceeds in analogy to the methods described in Process A, Step 5, by reaction of (XX) with a suitable acid chloride in the presence of suitable bases, for example pyridine, triethylamine or potassium carbonate, in a suitable inert solvent, for example dichloromethane.

### Process F

The inventive compounds of the formula (I) shown in Process E, in which V¹ or V² or both (V¹ and V²) are sulphur, can be prepared by methods known from the literature according to the following scheme:

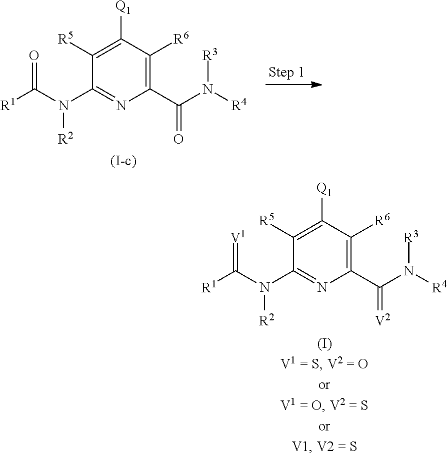

The R¹, R², R³, R⁴, R⁵, R⁶ and Q¹ radicals have the definitions described above. V¹ and V² have the definitions given in the scheme.

### Step 1

Thioamides of the formula (I) in which V¹ or V² or both (V¹ and V²) are sulphur can be prepared from amides of the formula (I-c) by reaction with a suitable thionating reagent, for example Lawesson's reagent (cf, in analogy, for example, WO2005/9435) or P₄S₁₀ (cf., in analogy, for example, European Journal of Medicinal Chemistry 1995 30, 915-924), in suitable solvents, for example toluene or xylene.

Alternatively, the thioamide synthesis can also proceed from the intermediates (VII) or (XVII). The further conversion to compounds of the formula (I) in which V¹ or V² or both (V¹ and V²) are sulphur is then effected analogously to the processes described in Processes A and C.

### Process G

The inventive compounds of the formula (I) shown in Process G, in which R² or R³ is C(O)Oalkyl and the other in each case is H or in which both (R² and R³) are C(O)Oalkyl can be prepared by methods known from the literature according to the following scheme:

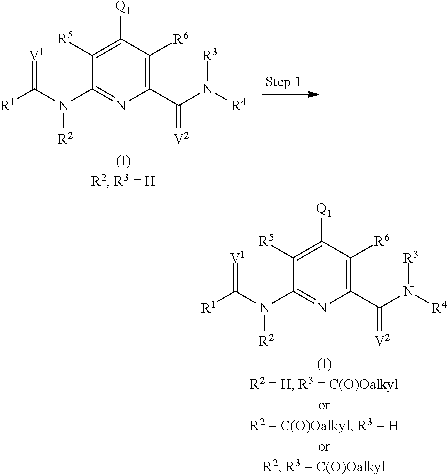

The R¹, R⁴, R⁵, R⁶, V¹, V² and Q¹ radicals have the definitions described above. R² and R³ have the definitions given in the scheme.

### Step 1

Carbamates of the formula (I) in which R² or R³ is C(O)Oalkyl and the other in each case is H or in which both (R² and R³) are C(O)Oalkyl can be prepared from amides of the formula (I) in which R² and R³ are H by reaction with chloroformic esters and a base, for example sodium hydride (cf., in analogy, EP1932836), in suitable solvents, for example DMF or tetrahydrofuran.

### Process H

The inventive compounds of the formula (I-c) shown in Process H can be prepared by the sequence of methods known from the literature according to the following scheme:

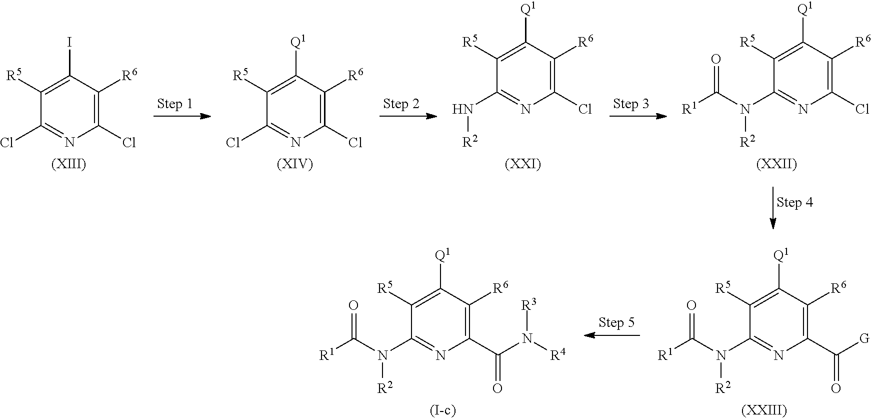

The R¹, R², R³, R⁴, R⁵, R⁶ and Q¹ radicals have the definitions described above. The G radical is alkoxy (especially methoxy and ethoxy) or NH₂.

### Step 1:

The 2,6-dichloro-4-iodopyridines of the formula (XIII) can be coupled at the iodine position to aromatic or heteroaromatic boronic acids, boronic esters or bisoxoborolanes to give compounds of the formula (XIV) by the known methods described in Process A, Step 9a, using suitable catalysts, for example tetrakis(triphenylphosphine)palladium(0). Some of the boronic acid derivatives or boronic ester derivatives required are known and/or commercially available, or they can be prepared by commonly known methods (cf. Boronic Acids (eds.: D. G. Hall), 2nd ed., Wiley-VCH, Weinheim, 2011).

Pyridine derivatives of the formula (XIII) can be coupled to NH-containing heteroaromatics analogously to the methods described in Process B.

### Step 2:

To obtain compounds of the formula (XXI) from the precursors (XIV), it is likewise possible to use methods known from the literature according to Process C, Step 5. For example, it is possible to introduce primary amines or ammonia (when R²=H) by direct reaction of the two coreactants by heating in a suitable solvent, for example water or dimethyl sulphoxide.

### Step 3:

The preparation of the compounds of the formula (XXII) proceeds in analogy to the methods described in Process A, Step 5, by reaction of (XXI) with a suitable acid chloride in the presence of suitable bases, for example pyridine, triethylamine or potassium carbonate, in a suitable inert solvent, for example dichloromethane, or with carboxylic acids in the presence of suitable coupling reagents, for example HATU or EDCI, and suitable bases, for example triethylamine or N,N-diisopropylethylamine, in suitable inert solvents, for example DMF or dichloromethane.

### Step 4:

Chloropyridines of the general formula (XXII) can be converted to compounds of the formula (XXIII) by reaction with carbon monoxide by processes known from the literature. When G=alkoxy, this reaction can be effected in a suitable alcohol (especially methanol or ethanol), by catalysis with tris(dibenzylideneacetone)dipalladium(0) [cf., for example, US2013/165464] or palladium(II) diacetate [cf., for example, WO2011/137342 or WO2014/114185] in the presence of a phosphine ligand, e.g. Xantphos (4,5-bis(diphenylphosphino)-9,9-dimethylxanthene) or Xphos (2-dicyclohexylphosphino-2′,4′,6′-triisopropylbiphenyl), and a suitable organic or inorganic base, e.g. triethylamine or caesium carbonate. When G=NH₂, the reaction can be conducted in the presence of ammonia, ammonia solution in water or an organic solvent, e.g. dioxane, also under transition metal catalysis [cf., for example, WO2000/037428; Chem. Eur. J. 2010, 16(32), 9750-9753].

### Step 5:

The resultant esters of the formula (XXIII) (G=alkoxy) can be transformed to the corresponding amides of the formula (I-c), for example in analogy to the methods described in Process A, Steps 6 and 7, by hydrolysis and activation of the carboxylic acid by coupling reagents or conversion to the corresponding carbonyl chloride.

It is possible to use the primary amides of the formula (XXIII) (G=NH₂) to prepare the corresponding imides of the formula (I-c) in analogy to the methods described in Process A, Step 5, by reaction with a suitable acid chloride in the presence of suitable bases, for example pyridine, triethylamine or potassium carbonate, in a suitable inert solvent, for example dichloromethane, or with carboxylic acids in the presence of suitable coupling reagents, for example HATU or EDCI, and suitable bases, for example triethylamine or N,N-diisopropylethylamine, in suitable inert solvents, for example DMF or dichloromethane.

Process I

The inventive compounds of the formula (I-c) shown in Process I can also be prepared by the sequence of methods known from the literature according to the following scheme:

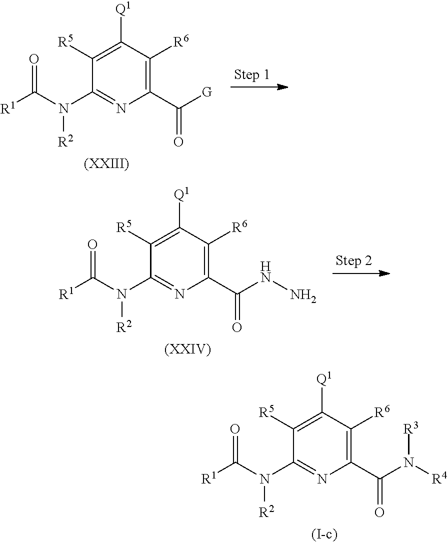

The R¹, R², R³, R⁴, R⁵, R⁶ and Q¹ radicals have the definitions described above. The G radical is alkoxy (especially methoxy).

### Step 1:

The preparation of compounds of the formula (XXIV) proceeds by reaction of esters of the formula (XXIII) with hydrazine hydrate in a suitable inert solvent, for example methanol, ethanol or dioxane, for example analogously to the methods described in WO2005/121152, European Journal of Medicinal Chemistry 2015, 93, 511-522 or Asian Journal of Chemistry 2015, 27, 4579-4582.

### Step 2:

Hydrazide derivatives of the formula (XXIV) can be converted to the corresponding imines of the formula (I-c) in analogy to the processes described in WO2010/72781 or Bioorganic and Medicinal Chemistry 2011, 19, 6400-6408, by condensation with an aldehyde in an alcoholic solvent, for example methanol or ethanol, or in a solvent mixture with water. For the condensation, an acid is usually used, for example sulphuric acid, hydrochloric acid or acetic acid.

### Process J

The inventive compounds of the formula (I-d) shown in Process J, in which V¹ is oxygen and V² is NH, can be prepared by the sequence of methods known from the literature according to the following scheme:

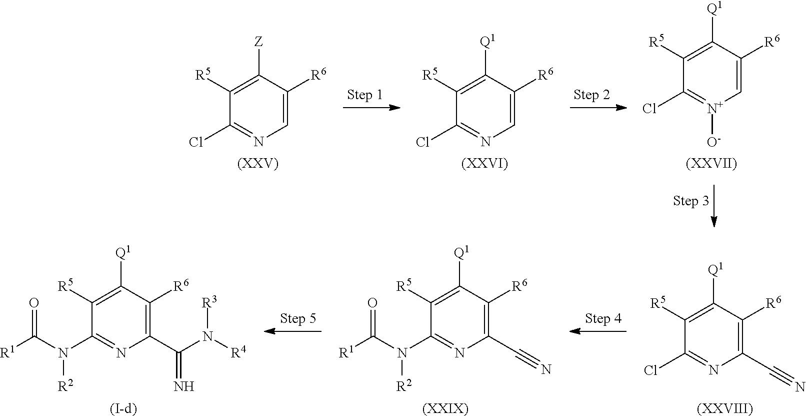

The R¹, R², R³, R⁴, R⁵, R⁶ and Q¹ radicals have the definitions described above.

Z is bromine, iodine or

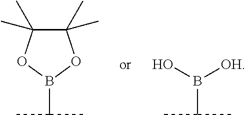

### Step 1:

The pyridine derivatives of the formula (XXV) can be coupled to (het)aryl halides or to aromatic or heteroaromatic boronic acids, boronic esters or bisoxoborolanes to give compounds of the formula (XXVI) by the known methods described in Process A, Step 9 and Step 9a, using suitable catalysts, for example tetrakis(triphenylphosphine)palladium(0). Some of the boronic acid derivatives or boronic ester derivatives required are known and/or commercially available, or they can be prepared by commonly known methods (cf. Boronic Acids (eds.: D. G. Hall), 2nd ed., Wiley-VCH, Weinheim, 2011).

Pyridine derivatives of the formula (XXV) (Z=iodine) can be coupled to NH-containing heteroaromatics analogously to the methods described in Process B.

Pyridine derivatives of the formula (XXV) are commercially available, for example 4-bromo-2-chloropyridine (R⁵=R⁶=hydrogen), 2-chloro-4-iodopyridine (R⁵=R⁶=hydrogen), 2-chloro-4-iodo-5-trifluoromethylpyridine (R⁵=hydrogen, R⁶=trifluoromethyl), 2,5-dichloro-4-iodopyridine (R⁵=hydrogen, R⁶=chlorine), 2-chloro-5-cyano-4-iodopyridine (R⁵=hydrogen, R⁶=cyano), 2,3-dichloro-4-iodopyridine (R⁵=chlorine, R⁶=hydrogen), 2-chloro-3-cyano-4-iodopyridine (R⁵=cyano, R⁶=hydrogen) or known from the literature or can be prepared by known processes (cf. WO2015/52264, Journal of Medicinal Chemistry 2012, 55, 4286-4296 or Tetrahedron 2002, 58, 4369-4373).

### Step 2:

Compounds of the formula (XXVI) can be converted to compounds of the formula (XXVII) by reaction with an oxidizing agent. Oxidizing agents used are frequently hydrogen peroxide in combination with an acid, for example trifluoroacetic acid, acetic acid or formic acid (cf. WO2010/25451 or Organometallics 2011, 30, 6751-6765) or 3-chloroperbenzoic acid in an inert solvent, for example dichloromethane or chloroform (cf. WO2011/40629, WO2010/109005 or WO2011/25505).

### Step 3:

By reaction of compounds of the formula (XXVII) with trimethylsilyl cyanide and an amine base, for example triethylamine in a suitable inert solvent, for example acetonitrile, in analogy to the processes described in, for example, WO2009/111337 or US2008/275057, it is possible to obtain compounds of the formula (XXVIII).

### Step 4:

Compounds of the formula (XXVIII) can be converted to compounds of the formula (XXIX) by reaction with a carboxamide using suitable catalysts, for example palladium(II) acetate or tris(dibenzylideneacetone)dipalladium(0), a phosphine ligand, for example Xantphos, and an inorganic base, for example caesium carbonate or potassium phosphate, in a suitable inert solvent, for example dioxane, in analogy to the processes described in, for example WO2011/137342, US2012/15943 or Bioorganic and Medicinal Chemistry Letters 2014, 24, 1148-1153.

### Step 5:

The corresponding amidines of the formula (I-d) can be obtained by reaction of compounds of the formula (XXIX) with amines in the presence of a Lewis acid, for example titanium tetrachloride or tin tetrachloride, optionally in a suitable inert solvent, for example tetrahydrofuran or dioxane, in analogy to the processes described in, for example, Tetrahedron Letters 2013, 54, 343-346 or Journal of Fluorescence 2014, 24, 1563-1570.

Alternatively, the reaction can be conducted in the presence of sodium methoxide in methanol (cf. Journal of Heterocyclic Chemistry 2011, 48, 921-926 or European Journal of Medicinal Chemistry 2013, 59, 7-14).

### PREPARATION EXAMPLES

### By Process D

### Sodium 4-(4-chlorophenyl)-2-oxobut-3-enoate

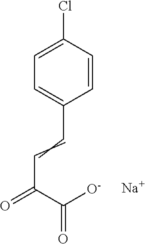 [SMILES: O=C([O-])C(=O)C=Cc1ccc(Cl)cc1 ; [Na+]]

5 g (35.57 mmol) of 4-chlorobenzaldehyde and 4.13 g (35.57 mmol) of ethyl 2-oxopropanoate were initially charged under protective gas, then, while cooling to 6-8° C., a solution of 3.13 g (78.25 mmol) of sodium hydroxide dissolved in 33 ml of water was added dropwise. The mixture was stirred for a further hour, then the reaction mixture was added to 300 ml of ethanol and the solids obtained were filtered off using a fluted filter. 7.130 g (86% of theory) of crude product were obtained, which were converted in the subsequent step without further purification.

### 6-Amino-4-(4-chlorophenyl)pyridine-2-carboxylic acid (XIX-1)

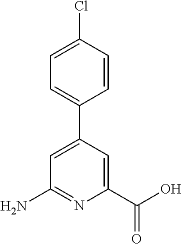 [SMILES: Nc1cc(-c2ccc(Cl)cc2)cc(C(=O)O)n1]

7.13 g (30.65 mmol) of sodium 4-(4-chlorophenyl)-2-oxobut-3-enoate and 4.74 g (30.65 mmol) of 1-(cyanomethyl)pyridinium chloride were added to 160 ml of glacial acetic acid, 5.06 g (65.6 mmol) of ammonium acetate were added, and the mixture was stirred at 80° C. for two hours. After cooling, the mixture was added to water and the reaction product was filtered off in solid form using a fluted filter. 5.70 g (68.8% of theory) of 6-amino-4-(4-chlorophenyl)pyridine-2-carboxylic acid (XIX-1) were obtained.

log P (acidic): 1.02; MH⁺: 249; ¹H-NMR (400 MHz, D₆-DMSO) δ ppm: 6.55 (broad, 2H), 6.93 (d, 1H), 7.43 (d, 1H), 7.57 (d, 2H), 7.72 (d, 2H).

### 6-Amino-4-(4-chlorophenyl)-N-(4-cyanobenzyl)pyridine-2-carboxamide (XX-1)

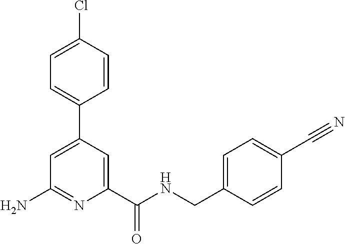 [SMILES: N#Cc1ccc(CNC(=O)c2cc(-c3ccc(Cl)cc3)cc(N)n2)cc1]

200 mg (0.80 mmol) of 6-amino-4-(4-chlorophenyl)pyridine-2-carboxylic acid (XIX-1), 295 mg (2.41 mmol) of 4-dimethylaminopyridine and 446.6 mg (2.41 mmol) of EDC hydrochloride were stirred in 65 ml of dichloromethane at room temperature for 15 minutes, then 106 mg (0.80 mmol) of 4-(aminomethyl)benzonitrile were added and the mixture was stirred at room temperature for a further 18 hours. For workup, the reaction mixture was added to about 100 ml of a 3% aqueous sodium dihydrogenphosphate solution. The mixture was extracted three times with dichloromethane, and the combined organic phases were dried over sodium sulphate and filtered. The solvent was distilled off under reduced pressure and the residue was purified by column chromatography purification using a water/acetonitrile gradient as the eluent. This gave 40 mg (13.7% of theory) of the target product (XX-1).

log P (neutral): 2.82; MH⁺: 363; ¹H-NMR (600 MHz, D₆-DMSO) δ ppm: 4.57 (d, 2H), 6.24 (broad, 2H), 6.91 (d, 1H), 7.44 (d, 1H), 7.50 (d, 2H), 7.58 (d, 2H), 7.70 (d, 2H), 7.80 (d, 2H), 8.96 (m, 1H).

### 4-(4-Chlorophenyl)-N-(4-cyanobenzyl)-6-[(cyclopropylcarbonyl)amino]pyridine-2-carboxamide (I-020)

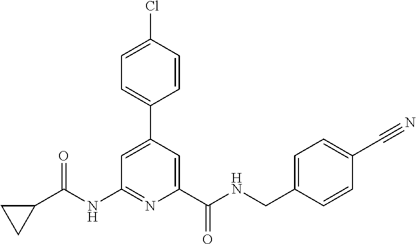 [SMILES: N#Cc1ccc(CNC(=O)c2cc(-c3ccc(Cl)cc3)cc(NC(=O)C3CC3)n2)cc1]

30 mg (0.08 mmol) of 6-amino-4-(4-chlorophenyl)-N-(4-cyanobenzyl)pyridine-2-carboxamide (XX-1) were first added to a mixture of 5 ml of dichloromethane and 5 ml of pyridine, and then, after cooling to 0° C., 35 mg (0.33 mmol) of cyclopropanecarbonyl chloride were added. The reaction mixture was stirred at room temperature for 48 hours and applied directly to Polygoprep 100-50 Cis. After column chromatography purification with a water/acetonitrile gradient as eluent, this gave 17 mg (46.5% of theory) of 4-(4-chlorophenyl)-N-(4-cyanobenzyl)-6-[(cyclopropylcarbonyl)amino]pyridine-2-carboxamide (I-020).

log P (neutral): 3.54; MH⁺: 431; ¹H-NMR (400 MHz, D₆-DMSO) δ ppm: 0.86 (m, 4H), 2.05 (m, 1H), 4.64 (d, 2H), 7.56 (d, 2H), 7.61 (d, 2H), 7.80 (m, 4H), 7.96 (d, 1H), 8.25 (d, 1H), 8.95 (tr, 1H), 10.96 (s, 1H).

### 6-Amino-N-cyclopropyl-N-methyl-4-[4-(trifluoromethyl)phenyl]pyridine-2-carboxamide (XX-2)

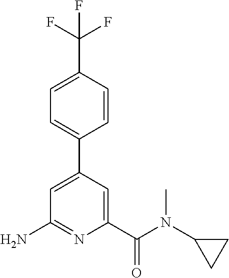 [SMILES: CN(C(=O)c1cc(-c2ccc(C(F)(F)F)cc2)cc(N)n1)C1CC1]

200 mg (0.71 mmol) of 6-amino-4-[4-(trifluoromethyl)phenyl]pyridine-2-carboxylic acid [synthesis analogous to 6-amino-4-(4-chlorophenyl)pyridine-2-carboxylic acid (XIX-1)], 260 mg (2.13 mmol) of 4-dimethylaminopyridine and 407.6 mg (2.13 mmol) of EDC hydrochloride were stirred in 80 ml of dichloromethane at room temperature for 15 minutes, then 50.4 mg (0.71 mmol) of N-methylcyclopropanamine were added and the mixture was stirred at room temperature for a further 18 hours. For workup, the reaction mixture was added to about 100 ml of a 3% aqueous sodium dihydrogenphosphate solution. The mixture was extracted three times with dichloromethane, and the combined organic phases were dried over sodium sulphate and filtered. The solvent was distilled off under reduced pressure and the residue was purified by column chromatography purification using a water/acetonitrile gradient as the eluent. This gave 49 mg (18% of theory) of the target product (XX-2).

log P (neutral): 2.40; MH⁺: 336; ¹H-NMR (400 MHz, D₆-DMSO) δ ppm: 0.48 (s br, 4H), 2.8-2.98 (m br, 4H), 6.30 (broad, 2H), 6.77 (s, 1H), 6.92 (s, 1H), 7.87 (m, 4H).

### N-Cyclopropyl-6-[(cyclopropylcarbonyl)amino]-N-methyl-4-[4-(trifluoromethyl)phenyl]pyridine-2-carboxamide (I-036)

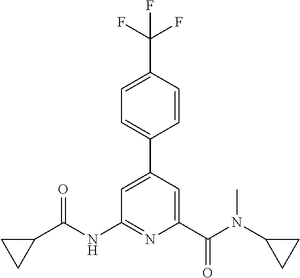 [SMILES: CN(C(=O)c1cc(-c2ccc(C(F)(F)F)cc2)cc(NC(=O)C2CC2)n1)C1CC1]

63 mg (0.19 mmol) of 6-amino-N-cyclopropyl-N-methyl-4-[4-(trifluoromethyl)phenyl]pyridine-2-carboxamide were first added to a mixture of 5 ml of dichloromethane and 5 ml of pyridine, and then, after cooling to 0° C., 79 mg (0.75 mmol) of cyclopropanecarbonyl chloride were added. The reaction mixture was stirred at room temperature for 48 hours and applied directly to Polygoprep 100-50 C₁₈. After column chromatography purification with a water/acetonitrile gradient as eluent, this gave 50 mg (65% of theory) of N-cyclopropyl-6-[(cyclopropylcarbonyl)amino]-N-methyl-4-[4-(trifluoromethyl)phenyl]pyridine-2-carboxamide (I-036).

log P (neutral): 3.21; MH⁺: 404; ¹H-NMR (400 MHz, D₆-DMSO) δ ppm: 0.47 (m, 4H), 0.86 (m, 4H), 2.03 (m, 1H), 2.8-3.1 (m, 4H), 7.59 (s, 1H), 7.90 (d, 2H), 7.98 (d, 2H), 8.49 (s, 1H), 11.10 (s, 1H).

### 6-Amino-N-(4-cyano-3-fluorobenzyl)-4-[4-(trifluoromethyl)phenyl]pyridine-2-carboxamide (XX-3)

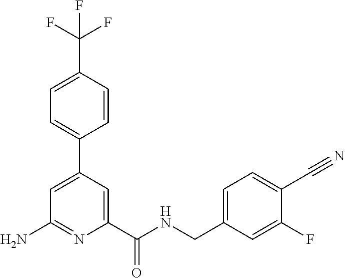 [SMILES: N#Cc1ccc(CNC(=O)c2cc(-c3ccc(C(F)(F)F)cc3)cc(N)n2)cc1F]

200 mg (0.71 mmol) of 6-amino-4-[4-(trifluoromethyl)phenyl]pyridine-2-carboxylic acid [synthesis analogous to 6-amino-4-(4-chlorophenyl)pyridine-2-carboxylic acid (XIX-1)], 260 mg (2.13 mmol) of 4-dimethylaminopyridine and 409 mg (2.13 mmol) of EDC hydrochloride were stirred in 65 ml of dichloromethane at room temperature for 15 minutes, then 106.4 mg (0.71 mmol) of 4-(aminomethyl)-2-fluorobenzonitrile were added and the mixture was stirred at room temperature for a further 18 hours. For workup, the reaction mixture was added to about 100 ml of a 3% aqueous sodium dihydrogenphosphate solution. The mixture was extracted three times with dichloromethane, and the combined organic phases were dried over sodium sulphate and filtered. The solvent was distilled off under reduced pressure and the residue was purified by column chromatography purification using a water/acetonitrile gradient as the eluent. This gave 62 mg of the target product (XX-3).

log P (neutral): 3.19; MH⁺: 415; ¹H-NMR (400 MHz, D₆-DMSO) δ ppm: 4.59 (d, 2H), 6.3 (broad, 2H), 6.99 (s, 1H), 7.35 (d, 1H), 7.44 (d, 1H), 7.49 (s, 1H), 7.9 (m, 5H), 9.05 (tr, 1H).

### By Process E

### 6-[Bis(cyclopropylcarbonyl)amino]-N-(4-cyano-3-fluorobenzyl)-4-[4-(trifluoromethyl)phenyl]pyridine-2-carboxamide (I-028)

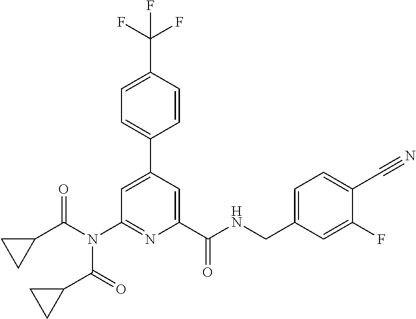 [SMILES: N#Cc1ccc(CNC(=O)c2cc(-c3ccc(C(F)(F)F)cc3)cc(N(C(=O)C3CC3)C(=O)C3CC3)n2)cc1F]

41.8 mg (0.1 mmol) of 6-amino-N-(4-cyano-3-fluorobenzyl)-4-[4-(trifluoromethyl)phenyl]pyridine-2-carboxamide (XX-3) were first added to a mixture of 5 ml of dichloromethane and 5 ml of pyridine, and then, after cooling to 0° C., 42 mg (0.4 mmol) of cyclopropanecarbonyl chloride were added. The reaction mixture was stirred at room temperature for 5 days and applied directly to Polygoprep 100-50 Cis. After column chromatography purification with a water/acetonitrile gradient as eluent, this gave 14 mg (24% of theory) of 6-[bis(cyclopropylcarbonyl)amino]-N-(4-cyano-3-fluorobenzyl)-4-[4-(trifluoromethyl)phenyl]pyridine-2-carboxamide (I-028).

log P (neutral/acidic): 4.18/4.27; MH⁺: 551; ¹H-NMR (400 MHz, D₆-DMSO) δ ppm: 0.91 (m, 4H), 1.04 (m, 4H), 2.05 (m, 2H), 4.61 (d, 2H), 7.37 (d, 1H), 7.45 (d, 1H), 7.9 (m, 3H), 8.15 (d, 2H), 8.23 (d, 1H), 8.42 (d, 1H), 9.51 (tr, 1H).

### By Process A

### Dimethyl 4-bromopyridine-2,6-dicarboxylate (III-1)

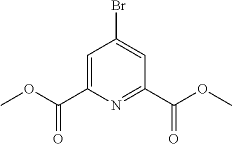

To a stirred suspension of 80.0 g (437 mmol) of 4-hydroxypyridine-2,6-dicarboxylic acid (II-1) in 1400 ml of carbon tetrachloride were added 752.0 g (1748 mmol) of phosphorus pentabromide. The resulting mixture was heated under reflux for 16 h and then quenched cautiously and dropwise with 350 ml of methanol at a temperature of <80° C. The mixture was stirred at 80° C. for another 1 h, cooled to room temperature and concentrated under reduced pressure. Ice-water was added to the residue, and it was stirred for another hour. The precipitating solids were filtered off, washed with water and dried by coevaporation with toluene. This gave 70.0 g (60.0% of theory) of the title compound (III-1).

1H-NMR (400.0 MHz, CDCl₃): δ=8.46 (s, 2H), 4.03 (s, 6H)

### 4-Bromo-6-(methoxycarbonyl)pyridine-2-carboxylic acid (IV-1)

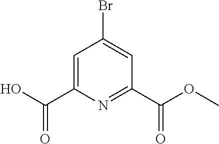

101.0 g (368 mmol) of dimethyl 4-bromopyridine-2,6-dicarboxylate (III-1) were suspended in 1000 ml of a 10:1 mixture (V/V) of methanol:dichloromethane, and 16.5 g (295 mmol) of potassium hydroxide were added. The reaction mixture was stirred at room temperature for 6 h. Subsequently, 1500 ml of diethyl ether were added to the mixture, which was stirred for a further 30 minutes. The precipitating solids were filtered off and washed with diethyl ether. Subsequently, the filter residue was resuspended and acidified to pH 3 with 2 N HCl. The precipitating solids were filtered off, washed with water and diethyl ether, and dried under reduced pressure. The filtrate was extracted four times with 200 ml each time of chloroform, and the combined organic phases were washed with brine, dried over sodium sulphate, filtered and concentrated under reduced pressure. The residue was combined with the solids obtained beforehand and the whole lot was dried by coevaporation with toluene. This gave 70.0 g (65.0% of theory) of the title compound (IV-1).

1H-NMR (400.0 MHz, d₆-DMSO): δ=13.83 (s, broad, 1H), 8.38 (d, 2H), 3.92 (s, 3H)

### Methyl 4-bromo-6-[(cyclopropylcarbonyl)amino]pyridine-2-carboxylate (VII-1)

### a) Methyl 4-bromo-6-[(tert-butoxycarbonyl)amino]pyridine-2-carboxylate (V-1)

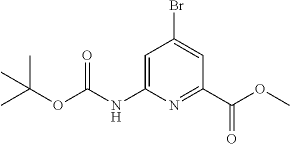 [SMILES: COC(=O)c1cc(Br)cc(NC(=O)OC(C)(C)C)n1]

To a mixture of 21.0 g (80.8 mmol) of 4-bromo-6-(methoxycarbonyl)pyridine-2-carboxylic acid (IV-1) and 13.1 g (129 mmol) of triethylamine in 280 ml of a mixture of t-butanol and 1,4-dioxane (ratio 1:3, V/V) were added 31.0 g (105 mmol) of diphenylphosphoryl azide at room temperature. The reaction mixture was then heated under reflux for 2 hours and then cooled to room temperature. Water was added and the mixture was extracted repeatedly with ethyl acetate. The combined organic phases were washed successively with water and brine, dried over sodium sulphate, filtered and concentrated. This gave 29 g of a crude fraction of the title compound (V-1), which was converted further directly to (VI-1) without further purification according to b).

### b) Methyl 6-amino-4-bromopyridine-2-carboxylate (VI-1)

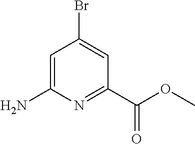

A solution of 70 g (assumed purity about 80% by TLC, about 165 mmol) of a prepared crude fraction of methyl 4-bromo-6-[(tert-butoxycarbonyl)amino]pyridine-2-carboxylate (V-1) in 400 ml of dichloromethane was cooled in an ice bath, and 200 ml of trifluoroacetic acid were slowly added dropwise. After the addition had ended, the mixture was stirred at room temperature for 3 h. Subsequently, the reaction mixture was concentrated under reduced pressure. The residue was diluted with ethyl acetate, adjusted to pH 11 with saturated sodium hydrogencarbonate solution, washed successively with water and brine, dried over sodium sulphate and filtered. The filtrate was concentrated under reduced pressure. This gave 40 g of a crude fraction of the title compound (VI-1), which was converted further directly to (VII-1) without further purification according to c).

### c) Methyl 4-bromo-6-[(cyclopropylcarbonyl)amino]pyridine-2-carboxylate (VII-1)

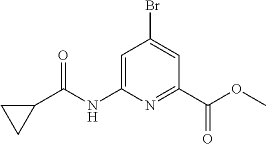 [SMILES: COC(=O)c1cc(Br)cc(NC(=O)C2CC2)n1]

To a stirred solution of 29.0 g (assumed purity 100%, 126 mmol) of a crude fraction of methyl 6-amino-4-bromopyridine-2-carboxylate (VI-1) in 300 ml of dry dichloromethane were added 20.0 g (251 mmol) of pyridine. After stirring at room temperature for 10 minutes, the mixture was cooled in an ice bath, and 16.0 g (151 mmol) of cyclopropanecarbonyl chloride were added. The reaction mixture was stirred at room temperature for a further three hours. Subsequently, the mixture was diluted with water and the phases were separated. The aqueous phase was extracted repeatedly with dichloromethane. The combined organic phases were washed successively with water and brine, dried over sodium sulphate, filtered and concentrated under reduced pressure. The residue was purified by chromatography using a silica gel column (ethyl acetate hexane, 2%-15%). This gave 20.0 g (54.0% of theory over the three stages a to c) of the title compound (VII-1).

1H-NMR (400.0 MHz, CDCl₃): δ=8.69 (s, 1H), 8.47 (s, 1H), 7.99 (s, 1H), 4.01 (s, 3H), 1.52 (m, 1H), 1.12 (m, 2H), 0.95 (m, 2H)

### 4-Bromo-6-[(cyclopropylcarbonyl)amino]pyridine-2-carboxylic acid (VIII-1)

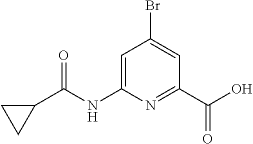 [SMILES: O=C(O)c1cc(Br)cc(NC(=O)C2CC2)n1]

To a stirred solution of 18.0 g (60.2 mmol) of methyl 4-bromo-6-[(cyclopropylcarbonyl)amino]pyridine-2-carboxylate (VII-1) in a mixture of 100 ml of THF and 100 ml of water were added 7.6 g (181 mmol) of lithium hydroxide monohydrate. The mixture was stirred at room temperature for 4 h. Subsequently, the THF was removed from the reaction mixture under reduced pressure. The aqueous phase was acidified with 4 N hydrochloric acid and the precipitating solids were filtered off. The solids were washed with water and dried by coevaporation with toluene. This gave 16.5 g (95.0% of theory) of the title compound (VIII-1).

1H-NMR (400.0 MHz, d₆-DMSO): δ=8.43 (s, 1H), 8.25 (s, 1H), 7.79 (s, 1H), 2.28 (m, 1H), 0.88 (d, 4H)

### 4-Bromo-N-(4-cyanobenzyl)-6-[(cyclopropylcarbonyl)amino]pyridine-2-carboxamide (IX-1)

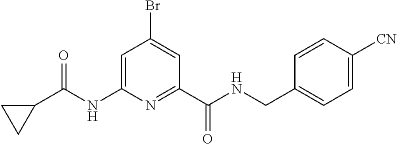 [SMILES: N#Cc1ccc(CNC(=O)c2cc(Br)cc(NC(=O)C3CC3)n2)cc1]

To a stirred solution of 17.0 g (43.8 mmol) of 4-bromo-6-[(cyclopropylcarbonyl)amino]pyridine-2-carboxylic acid (VIII-1) in 200 ml of dry dimethylformamide at room temperature were added 43.0 g (110 mmol) of HATU. After stirring for ten minutes, 8.1 g (48.2 mmol) of 4-(aminomethyl)benzonitrile hydrochloride and subsequently 17.0 g (132 mmol) of N,N-diisopropylethylamine were added. The mixture was stirred at room temperature for another 15 h. This was followed by dilution with water and ethyl acetate and separation of the phases. The aqueous phase was extracted repeatedly with ethyl acetate. The combined organic phases were washed successively with water, 1 N hydrochloric acid, brine and saturated sodium hydrogencarbonate solution, dried over sodium sulphate and filtered. The filtrate was concentrated under reduced pressure and the residue was purified by chromatography using a silica gel column (ethyl acetate/dichloromethane, 0 to 1:20). This gave 14.0 g (70.0% of theory) of the title compound (IX-1).

1H-NMR (400.0 MHz, CDCl₃): δ=8.61 (s, 1H), 8.11 (s, broad, 2H), 8.08 (s, 1H), 7.62 (m, 2H), 7.44 (m, 2H), 4.69 (d, 2H), 1.54 (m, 1H), 1.13 (m, 2H), 0.93 (m, 2H)

### N-(4-Cyanobenzyl)-6-[(cyclopropylcarbonyl)amino]-4-(4,4,5,5-tetramethyl-1,3,2-dioxaborolan-2-yl)pyridine-2-carboxamide (X-1)

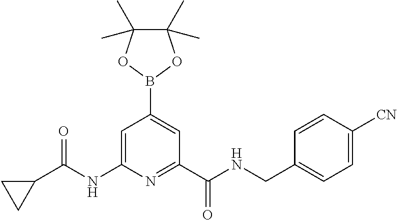 [SMILES: CC1(C)OB(c2cc(NC(=O)C3CC3)nc(C(=O)NCc3ccc(C#N)cc3)c2)OC1(C)C]

Alternative 1:

To a stirred and degassed solution of 9.2 g (23.1 mmol) of 4-bromo-N-(4-cyanobenzyl)-6-[(cyclopropylcarbonyl)amino]pyridine-2-carboxamide (IX-1), 7.3 g (28.8 mmol) of bis(pinacolato)diboron and 6.64 g (69.0 mmol) of potassium acetate in 100 ml of dry 1,4-dioxane under an argon atmosphere were added 920 mg (1.28 mmol) of [1,1′-bis(diphenylphosphino)ferrocene]palladium(II) dichloride. Subsequently, the mixture was stirred under an argon atmosphere at 85° C. for 16 h. Thereafter, the reaction solution was cooled to room temperature, filtered through Celite and washed through with dichloromethane. The filtrate was concentrated under reduced pressure and the residue was recrystallized from a mixture of hexane:dichloromethane (5:1, V/V). This gave 9.0 g (87.0% of theory) of the title compound (X-1).

1H-NMR (400.0 MHz, CDCl₃): δ=8.69 (s, 1H), 8.29 (s, 1H), 8.16 (broad, 1H), 8.06 (broad, 1H), 7.63 (d, 2H), 7.45 (d, 2H), 4.71 (d, 2H), 1.55 (m, 1H), 1.32 (s, 12H), 1.13 (m, 2H), 0.92 (m, 2H)

Alternative 2:

To a stirred and degassed solution of 250 mg (0.62 mmol) of 4-bromo-N-(4-cyanobenzyl)-6-[(cyclopropylcarbonyl)amino]pyridine-2-carboxamide (IX-1), 191 mg (0.75 mmol) of bis(pinacolato)diboron and 184 mg (1.87 mmol) of potassium acetate in 4 ml of acetonitrile in a microwave reaction vessel under an argon atmosphere were added 23 mg (0.03 mmol) of [1,1′-bis(diphenylphosphino)ferrocene]palladium(II) dichloride. Subsequently, the mixture was heated to 150° C. in a Biotage Initiator microwave for 15 minutes. Thereafter, the reaction solution was cooled to room temperature, filtered through a silica gel/sodium sulphate cartridge and washed through with acetonitrile and ethyl acetate. The filtrate was concentrated under reduced pressure and the residue was subjected to acidic chromatographic separation by means of MPLC (reversed phase; eluent: water/acetonitrile). This gave 190 mg (75% of theory) of {2-[(4-cyanobenzyl)carbamoyl]-6-[(cyclopropylcarbonyl)amino]pyridin-4-yl}boric acid (X-1b) as the hydrolysis product of the title compound (X-1).

Further conversion by this method resulted regularly in mixtures of (X-1) with the boric acid derivative (X-1b) in varying ratios.

### {2-[(4-Cyanobenzyl)carbamoyl]-6-[(cyclopropylcarbonyl)amino]pyridin-4-yl}boric acid (X-1b)

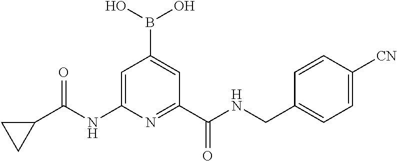 [SMILES: N#Cc1ccc(CNC(=O)c2cc(B(O)O)cc(NC(=O)C3CC3)n2)cc1]

log P (neutral/acidic): 1.25/1.58; MH⁺: 365; 1H-NMR (400.0 MHz, d₆-DMSO): δ=10.679 (0.9); 8.843 (0.5); 8.559 (0.4); 8.496 (1.3); 8.090 (1.3); 7.829 (1.2); 7.809 (1.5); 7.532 (1.2); 7.512 (1.1); 4.631 (1.0); 4.616 (1.0); 3.569 (16.0); 3.362 (0.6); 2.507 (14.0); 2.503 (18.4); 2.499 (13.9); 1.987 (0.4); 1.355 (0.3); 1.320 (0.5); 0.834 (2.0); 0.823 (1.2); 0.814 (1.0); 0.000 (5.3)

### N-(4-Cyanobenzyl)-6′-[(cyclopropylcarbonyl)amino]-6-(trifluoromethyl)-3,4′-bipyridine-2′-carboxamide (I-123)

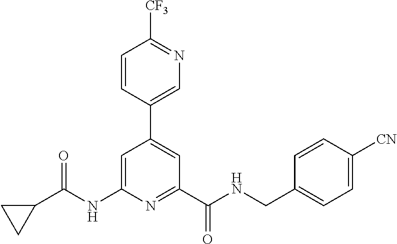 [SMILES: N#Cc1ccc(CNC(=O)c2cc(-c3ccc(C(F)(F)F)nc3)cc(NC(=O)C3CC3)n2)cc1]

200 mg (assumed purity 100%, 0.55 mmol) of the {2-[(4-cyanobenzyl)carbamoyl]-6-[(cyclopropylcarbonyl)amino]pyridin-4-yl}boric acid (X-1b) obtained according to Alternative 2, 113 mg (0.50 mmol) of 5-bromo-2-(trifluoromethyl)pyridine and 106 mg (1.0 mmol) of sodium carbonate were weighed into a microwave vessel. Subsequently, 8 ml of 1,4-dioxane and 1.5 ml of water were added and the mixture was degassed. The vial was flooded with argon, and 17.4 mg (0.01 mmol) of tetrakis(triphenylphosphine)palladium(0) were added. The mixture was heated in an IR Discover microwave at 130° C. for 45 minutes. Thereafter, the reaction solution was cooled to room temperature, filtered through a silica gel/sodium sulphate cartridge and washed through with 1,4-dioxane. The filtrate was concentrated under reduced pressure and the residue was purified by means of HPLC separation. This gave 76 mg (100% purity, 32.7% of theory) of the title compound (1-123).

log P (acidic): 3.02; MH⁺: 466; 1H-NMR (400.0 MHz, d₆-DMSO): δ=11.059 (5.6); 9.159 (4.8); 9.154 (4.9); 9.006 (1.5); 8.990 (3.2); 8.975 (1.6); 8.585 (7.7); 8.581 (7.9); 8.483 (2.4); 8.478 (2.4); 8.463 (2.6); 8.458 (2.7); 8.317 (4.9); 8.092 (8.6); 8.088 (8.7); 8.081 (5.4); 8.060 (4.6); 7.843 (8.8); 7.822 (10.3); 7.561 (8.8); 7.540 (7.6); 4.663 (6.3); 4.648 (6.4); 3.429 (0.3); 3.387 (0.5); 3.378 (0.6); 3.369 (0.7); 3.331 (645.6); 3.307 (2.7); 3.278 (0.5); 2.695 (0.3); 2.676 (3.7); 2.671 (5.2); 2.667 (3.9); 2.662 (2.0); 2.641 (0.4); 2.602 (0.4); 2.550 (1.2); 2.525 (13.1); 2.520 (20.4); 2.511 (287.6); 2.507 (600.0); 2.502 (797.7); 2.498 (587.3); 2.493 (292.3); 2.414 (0.5); 2.334 (3.7); 2.329 (5.2); 2.325 (3.8); 2.083 (0.6); 2.068 (1.6); 2.052 (2.3); 2.037 (1.8); 2.021 (0.7); 1.989 (0.9); 1.398 (1.4); 1.175 (0.5); 1.148 (0.6); 0.879 (16.0); 0.866 (8.7); 0.862 (8.5); 0.146 (5.0); 0.056 (0.4); 0.047 (0.5); 0.043 (0.4); 0.038 (0.4); 0.027 (0.7); 0.020 (1.5); 0.008 (38.1); 0.000 (1148.9); −0.008 (52.0); −0.023 (2.0); −0.031 (1.3); −0.033 (1.3); −0.041 (0.6); −0.045 (0.4); −0.052 (0.8); −0.150 (5.1)

### N-(4-Cyanobenzyl)-6-[(cyclopropylcarbonyl)amino]-4-[5-(trifluoromethyl)-2-thienyl]pyridine-2-carboxamide (I-160)

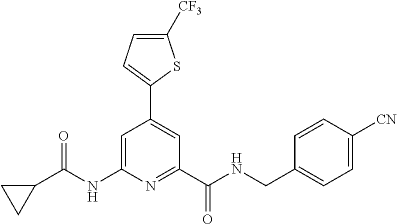 [SMILES: N#Cc1ccc(CNC(=O)c2cc(-c3ccc(C(F)(F)F)s3)cc(NC(=O)C3CC3)n2)cc1]

100 mg (assumed purity 100%, 0.27 mmol) of the {2-[(4-cyanobenzyl)carbamoyl]-6-[(cyclopropylcarbonyl)amino]pyridin-4-yl}boric acid (X-1b) obtained according to Alternative 2, 58 mg (0.25 mmol) of 2-bromo-5-(trifluoromethyl)thiophene and 53 mg (0.5 mmol) of sodium carbonate were weighed into a microwave vessel. Subsequently, 4.5 ml of 1,4-dioxane and 0.9 ml of water were added and the mixture was degassed. The vial was flooded with argon, and 8.7 mg (0.008 mmol) of tetrakis(triphenylphosphine)palladium(0) were added. The mixture was heated in an IR Discover microwave at 130° C. for 45 minutes. Thereafter, the reaction solution was cooled to room temperature, filtered through a silica gel/sodium sulphate cartridge and washed through with 1,4-dioxane. The filtrate was concentrated under reduced pressure and the residue was purified by means of HPLC separation. This gave 29 mg (100% purity, 24.9% of theory) of the title compound (1-160).

log P (neutral/acidic): −/3.73; MH⁺: 471; 1H-NMR (400.0 MHz, d₆-DMSO): δ=11.030 (7.7); 10.929 (0.4); 8.948 (2.2); 8.932 (4.6); 8.916 (2.2); 8.589 (0.6); 8.551 (9.4); 8.548 (10.0); 8.511 (0.6); 8.266 (0.5); 8.059 (0.6); 8.024 (10.0); 8.020 (10.4); 7.941 (4.3); 7.933 (5.0); 7.838 (15.6); 7.817 (14.1); 7.553 (12.7); 7.532 (11.0); 4.650 (9.8); 4.634 (9.8); 3.469 (0.4); 3.453 (0.4); 3.432 (0.6); 3.426 (0.5); 3.398 (0.9); 3.326 (1198.6); 3.284 (0.8); 3.271 (0.7); 3.260 (0.6); 3.237 (0.4); 2.711 (0.7); 2.675 (3.1); 2.671 (4.2); 2.667 (3.2); 2.606 (0.4); 2.541 (145.4); 2.524 (12.7); 2.506 (496.4); 2.502 (656.3); 2.498 (502.1); 2.408 (0.3); 2.367 (0.7); 2.333 (3.0); 2.328 (4.1); 2.324 (3.2); 2.067 (0.7); 2.049 (2.0); 2.044 (1.7); 2.036 (3.3); 2.023 (2.1); 2.005 (0.9); 1.298 (0.4); 1.259 (0.6); 1.235 (1.2); 1.187 (0.4); 0.904 (0.5); 0.879 (16.0); 0.872 (12.3); 0.860 (8.9); 0.837 (0.7); 0.008 (1.5); 0.000 (45.1)

### N-(4-Cyanobenzyl)-6′-[(cyclopropylcarbonyl)amino]-5-(trifluoromethyl)-2,4′-bipyridine-2′-carboxamide (I-174)

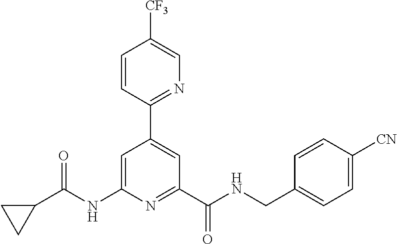 [SMILES: N#Cc1ccc(CNC(=O)c2cc(-c3ccc(C(F)(F)F)cn3)cc(NC(=O)C3CC3)n2)cc1]

240 mg (assumed purity 100%, 0.65 mmol) of the {2-[(4-cyanobenzyl)carbamoyl]-6-[(cyclopropylcarbonyl)amino]pyridin-4-yl}boric acid (X-1b) obtained according to Alternative 2 and 179 mg (0.79 mmol) of 2-bromo-5-(trifluoromethyl)pyridine were weighed into a microwave vessel. Subsequently, 4 ml of 1,4-dioxane and 0.75 ml of a two-molar aqueous sodium carbonate solution were added and the mixture was degassed. The vial was flooded with argon, and 22.8 mg (0.02 mmol) of tetrakis(triphenylphosphine)palladium(0) were added. The mixture was heated in an IR Discover microwave at 130° C. for 45 minutes. Thereafter, the reaction solution was cooled to room temperature, diluted with ethyl acetate and filtered through a silica gel/sodium sulphate cartridge and washed through with ethyl acetate. The filtrate was concentrated under reduced pressure and the residue was subjected to chromatographic purification by means of MPLC (eluent:cyclohexane:ethyl acetate 1:8). This gave 110 mg (95.0% purity, 34.1% of theory) of the title compound (1-174).

log P (neutral/acidic): 3.22/3.25; MH⁺: 466; 1H-NMR (400.0 MHz, d₆-DMSO): δ=11.012 (8.2); 9.164 (7.4); 8.994 (11.4); 8.991 (11.9); 8.970 (2.2); 8.955 (4.6); 8.939 (2.2); 8.409 (3.0); 8.398 (12.9); 8.394 (13.3); 8.389 (5.7); 8.383 (5.3); 8.339 (7.9); 8.318 (4.4); 7.842 (13.3); 7.821 (15.9); 7.808 (0.6); 7.565 (13.7); 7.544 (11.9); 7.514 (0.4); 4.671 (10.3); 4.655 (10.3); 4.626 (0.3); 4.613 (0.4); 3.410 (12.1); 3.356 (18.0); 2.676 (0.9); 2.672 (1.3); 2.668 (1.0); 2.525 (3.0); 2.507 (166.3); 2.503 (222.3); 2.499 (162.9); 2.334 (0.9); 2.330 (1.3); 2.325 (1.0); 2.081 (0.8); 2.068 (1.8); 2.063 (2.1); 2.058 (1.7); 2.051 (3.6); 2.038 (2.2); 2.033 (2.1); 2.020 (1.0); 1.070 (1.8); 0.912 (0.6); 0.888 (11.5); 0.879 (16.0); 0.860 (8.6); 0.840 (1.0); 0.824 (0.3); 0.008 (0.7); 0.000 (21.9); −0.008 (0.8)

### N-(4-Cyanobenzyl)-6-[(cyclopropylcarbonyl)amino]-4-[2-ethyl-6-methyl-4-(pentafluoroethyl)phenyl]pyridine-2-carboxamide (I-148)

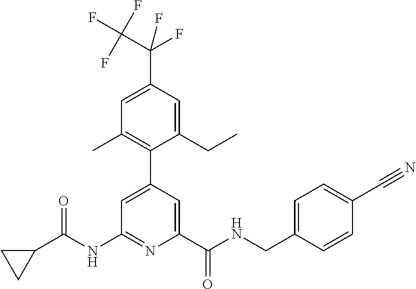 [SMILES: CCc1cc(C(F)(F)C(F)(F)F)cc(C)c1-c1cc(NC(=O)C2CC2)nc(C(=O)NCc2ccc(C#N)cc2)c1]

152 mg (0.34 mmol) of N-(4-cyanobenzyl)-6-[(cyclopropylcarbonyl)amino]-4-(4,4,5,5-tetramethyl-1,3,2-dioxaborolan-2-yl)pyridine-2-carboxamide (X-1), 108 mg (0.34 mmol) of 2-bromo-1-ethyl-3-methyl-5-(pentafluoroethyl)benzene (synthesis, for example, analogous to 4-bromo-2-fluoro-1-(pentafluoroethyl)benzene in WO2012010573) and 12 mg (0.01 mmol) of tetrakis(triphenylphosphine)palladium(0) were initially charged in a mixture of degassed dioxane (3.8 ml) and degassed sodium carbonate solution (1M, 1.4 ml), and the mixture was stirred at 92° C. for 14 h. Subsequently, the reaction mixture was cooled to room temperature and concentrated under reduced pressure, and the residue was taken up in dichloromethane and water. The phases were separated, the aqueous phase was extracted twice with dichloromethane and the combined organic phases were dried over magnesium sulphate and filtered. The solvent was distilled off under reduced pressure and the residue was purified by column chromatography purification using a cyclohexane/ethyl acetate gradient as eluent. This gave 38 mg (97% purity, 20% of theory) of the title compound (I-148).

log P (neutral/acidic): 4.72/4.85; MH⁺: 557; ¹H-NMR (400 MHz, D₆-DMSO) δ ppm: 0.82-0.84 (m, 4H), 0.99 (t, 3H), 1.99-2.08 (m, 4H), 2.32-2.42 (m, 2H), 4.64 (d, 2H), 7.49-7.57 (m, 5H), 7.82-7.84 (m, 2H), 8.04 (d, 1H), 8.92 (t, 1H), 11.00 (s, 1H).

### N-(4-Cyanobenzyl)-6-[(cyclopropylcarbonyl)amino]-4-[3-fluoro-4-(pentafluoroethyl)phenyl]pyridine-2-carboxamide (I-149)

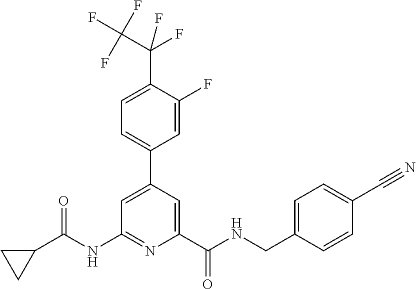 [SMILES: N#Cc1ccc(CNC(=O)c2cc(-c3ccc(C(F)(F)C(F)(F)F)c(F)c3)cc(NC(=O)C3CC3)n2)cc1]

150 mg (0.34 mmol) of N-(4-cyanobenzyl)-6-[(cyclopropylcarbonyl)amino]-4-(4,4,5,5-tetramethyl-1,3,2-dioxaborolan-2-yl)pyridine-2-carboxamide (X-1), 99 mg (0.34 mmol) of 4-bromo-2-fluoro-1-(pentafluoroethyl)benzene (synthesis: see WO2012010573) and 12 mg (0.01 mmol) of tetrakis(triphenylphosphine)palladium(0) were initially charged in a mixture of degassed dioxane (3.8 ml) and degassed sodium carbonate solution (1M, 1.3 ml), and the mixture was stirred at 92° C. for 14 h. Subsequently, the reaction mixture was cooled to room temperature and concentrated under reduced pressure, and the residue was taken up in dichloromethane and water. The phases were separated, the aqueous phase was extracted twice with dichloromethane and the combined organic phases were dried over magnesium sulphate and filtered. The solvent was distilled off under reduced pressure and the residue was purified by column chromatography purification using a cyclohexane/ethyl acetate gradient as eluent. This gave 53 mg (99% purity, 29% of theory) of the title compound (I-149).

log P (neutral/acidic): 4.14/4.25; MH⁺: 533; ¹H-NMR (400 MHz, D₆-DMSO) δ ppm: 0.86-0.88 (m, 4H), 2.03-2.06 (m, 1H), 4.65 (d, 2H), 7.54-7.56 (m, 2H), 7.82-8.00 (m, 5H), 8.05 (d, 1H), 8.56 (d, 1H), 8.97 (t, 1H), 11.03 (s, 1H).

### 4-[(1,1,2,2-Tetrafluoroethyl)sulphanyl]phenyl trifluoromethanesulphonate

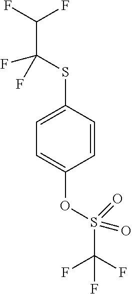 [SMILES: O=S(=O)(Oc1ccc(SC(F)(F)C(F)F)cc1)C(F)(F)F]

To an initial charge of 150 mg (0.66 mmol) of 4-[(1,1,2,2-tetrafluoroethyl)sulphanyl]phenol and 81 mg (0.79 mmol) of triethylamine in dichloromethane at 0° C. were added 225 mg (0.79 mmol) of trifluoromethanesulphonic anhydride. The mixture was stirred at room temperature for 14 h and then water was added. The phases were separated, the aqueous phase was extracted twice with dichloromethane and the combined organic phases were dried over magnesium sulphate and filtered. The solvent was distilled off under reduced pressure. The crude product was used without further purification in the next stage.

### N-(4-Cyanobenzyl)-6-[(cyclopropylcarbonyl)amino]-4-{4-[(1,1,2,2-tetrafluoroethyl)sulphanyl]phenyl}pyridine-2-carboxamide (I-178)

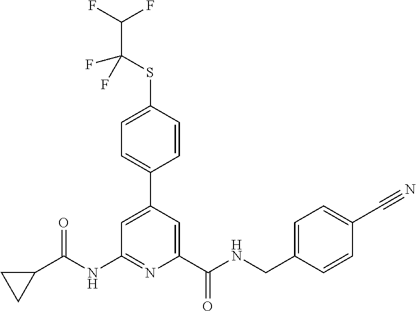 [SMILES: N#Cc1ccc(CNC(=O)c2cc(-c3ccc(SC(F)(F)C(F)F)cc3)cc(NC(=O)C3CC3)n2)cc1]

150 mg (0.34 mmol) of N-(4-cyanobenzyl)-6-[(cyclopropylcarbonyl)amino]-4-(4,4,5,5-tetramethyl-1,3,2-dioxaborolan-2-yl)pyridine-2-carboxamide (X-1), 120 mg (0.34 mmol) of 4-[(1,1,2,2-tetrafluoroethyl)sulphanyl]phenyl trifluoromethanesulphonate and 12 mg (0.01 mmol) of tetrakis(triphenylphosphine)palladium(0) were initially charged in a mixture of degassed dioxane (3.8 ml) and degassed sodium carbonate solution (1M, 1.3 ml), and the mixture was stirred at 92° C. for 14 h. Subsequently, the reaction mixture was cooled to room temperature and concentrated under reduced pressure, and the residue was taken up in dichloromethane and water. The phases were separated, the aqueous phase was extracted twice with dichloromethane and the combined organic phases were dried over magnesium sulphate and filtered. The solvent was distilled off under reduced pressure and the residue was purified by column chromatography purification using a cyclohexane/ethyl acetate gradient as eluent. This gave 69 mg (98% purity, 38% of theory) of the title compound (1-178).

log P (acidic): 3.81; MH⁺: 529; ¹H-NMR (400 MHz, D₆-DMSO) δ ppm: 0.85-0.87 (m, 4H), 2.01-2.07 (m, 1H), 4.65 (d, 2H), 6.62-6.90 (m, 1H), 7.54-7.56 (m, 2H), 7.82-7.91 (m, 6H), 8.00 (d, 1H), 8.56 (d, 1H), 8.94 (t, 1H), 10.97 (s, 1H).

### N-(4-Cyanobenzyl)-6-[(cyclopropylcarbonyl)amino]-4-[4-(trifluoromethoxy)phenyl]pyridine-2-carboxamide (I-044)

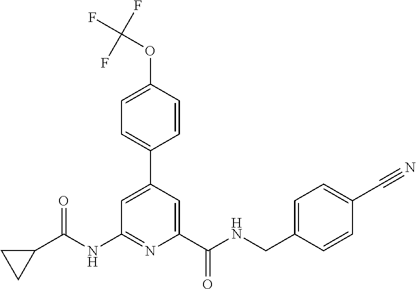 [SMILES: N#Cc1ccc(CNC(=O)c2cc(-c3ccc(OC(F)(F)F)cc3)cc(NC(=O)C3CC3)n2)cc1]

500 mg (1.25 mmol) of 4-bromo-N-(4-cyanobenzyl)-6-[(cyclopropylcarbonyl)amino]pyridine-2-carboxamide (IX-1), 258 mg (1.25 mmol) 4-(trifluoromethoxy)phenylboronic acid and 43 mg (0.04 mmol) of tetrakis(triphenylphosphine)palladium(0) were initially charged in a mixture of degassed dioxane (12 ml) and degassed sodium carbonate solution (1M, 5 ml), and the mixture was stirred at 96° C. for 14 h. Subsequently, the reaction mixture was cooled to room temperature and concentrated under reduced pressure, and the residue was taken up in dichloromethane and water. The phases were separated, the aqueous phase was extracted twice with dichloromethane and the combined organic phases were dried over magnesium sulphate and filtered. The solvent was distilled off under reduced pressure and the residue was purified by column chromatography purification using a water/acetonitrile gradient as the eluent. This gave 193 mg (100% purity, 32% of theory) of the title compound (1-044).

log P (neutral/acidic): 3.76/3.90; MH⁺: 481; ¹H-NMR (400 MHz, D₆-DMSO) δ ppm: 0.85-0.87 (m, 4H), 2.01-2.07 (m, 1H), 4.65 (d, 2H), 7.52-7.56 (m, 4H), 7.82-7.84 (m, 2H), 7.89-7.92 (m, 2H), 7.98 (d, 1H), 8.53 (d, 1H), 8.94 (t, 1H), 10.97 (s, 1H).

### Dimethyl 4-chloropyridine-2,6-dicarboxylate (IIa-1)

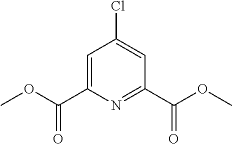

To a stirred suspension of 100.0 g (546 mmol) of 4-hydroxypyridine-2,6-dicarboxylic acid (II-1) in 1500 ml of carbon tetrachloride were added 455.0 g (2184 mmol) of phosphorus pentachloride. The resulting mixture was heated under reflux for 16 h and then quenched cautiously and dropwise with 375 ml of methanol at a temperature of <80° C. The mixture was stirred under reflux for another 1 h, cooled to room temperature and concentrated under reduced pressure. Ice-water was added to the residue, and it was stirred for another hour. The precipitating solids were filtered off, washed with water and dried under reduced pressure. This gave 72.0 g (57.0% of theory) of the title compound (IIa-1).

1H-NMR (400.0 MHz, CDCl₃): δ=8.30 (s, 2H), 4.04 (s, 6H)

### Dimethyl 4-iodopyridine-2,6-dicarboxylate (III-2)

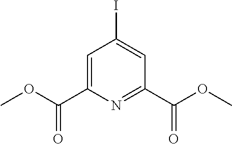

To a stirred mixture of 72.0 g (314 mmol) of dimethyl 4-chloropyridine-2,6-dicarboxylate (II-1) and 310 g of sodium iodide in 1500 ml of acetonitrile at 0° C. were added 66.0 ml (941 mmol) of acetyl chloride. The resulting mixture was heated to 50° C. for 4 h and then the acetonitrile was removed under reduced pressure. The residue was cautiously introduced into saturated sodium carbonate solution and extracted with dichloromethane. The combined organic phases were washed successively with saturated sodium thiosulphate solution and water, dried over sodium sulphate and concentrated. This gave 100.0 g (99.0% of theory) of the title compound (III-2).

1H-NMR (400.0 MHz, CDCl₃): δ=8.67 (s, 2H), 4.02 (s, 6H)

### 4-Iodo-6-(methoxycarbonyl)pyridine-2-carboxylic acid (IV-2)

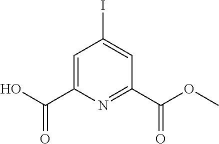

100.0 g (312 mmol) of dimethyl 4-iodopyridine-2,6-dicarboxylate (III-2) were suspended in 1000 ml of a 10:1 mixture (V/V) of methanol:dichloromethane, and 14.8 g (265 mmol) of potassium hydroxide were added. The reaction mixture was stirred at room temperature for 6 h. Subsequently, 1500 ml of diethyl ether were added to the mixture, which was stirred for a further 30 minutes. The precipitating solids were filtered off and washed with diethyl ether. Subsequently, the filter residue was resuspended and acidified to pH 3 with 3 N HCl. The precipitating solids were filtered off, washed successively with dichloromethane and diethyl ether, and dried under reduced pressure. This gave 56.0 g (58.0% of theory) of the title compound (IV-2).

1H-NMR (400.0 MHz, d₆-DMSO): δ=8.47 (s, 1H), 8.40 (s, 1H), 3.90 (s, 3H)

### Methyl 6-[(cyclopropylcarbonyl)amino]-4-iodopyridine-2-carboxylate (VII-2)

### a) Methyl 6-[(tert-butoxycarbonyl)amino]-4-iodopyridine-2-carboxylate (V-2)

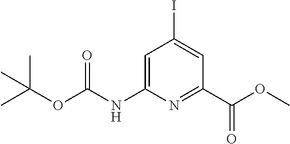 [SMILES: COC(=O)c1cc(I)cc(NC(=O)OC(C)(C)C)n1]

To a mixture of 50.0 g (163 mmol) of 4-iodo-6-(methoxycarbonyl)pyridine-2-carboxylic acid (IV-2) and 34 ml (244 mmol) of triethylamine in 600 ml of a mixture of t-butanol and 1,4-dioxane (ratio 1:3, V/V) were added 54.0 g (195 mmol) of diphenylphosphoryl azide at room temperature. The reaction mixture was then stirred at room temperature for 1 h, then heated under reflux for another 2 hours and then cooled to room temperature. Precipitating solids were filtered off. Water was added to the filtrate, and this aqueous phase was extracted repeatedly with ethyl acetate. The combined organic phases were washed successively with water, dried over sodium sulphate, filtered and concentrated. This gave 70 g of a crude fraction of the title compound (V-2), which was converted further directly to (VI-2) without further purification according to b).

### b) Methyl 6-amino-4-iodopyridine-2-carboxylate (VI-2)

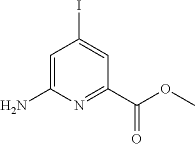

A solution of 70 g of the crude fraction of methyl 4-bromo-6-[(tert-butoxycarbonyl)amino]pyridine-2-carboxylate (V-2) prepared according to a) in 400 ml of dichloromethane was cooled in an ice bath, and 200 ml of trifluoroacetic acid were slowly added dropwise. After the addition had ended, the mixture was stirred at room temperature for 6 h. Subsequently, the reaction mixture was concentrated under reduced pressure. The residue was diluted with ethyl acetate and introduced into saturated sodium carbonate solution. After phase separation, the organic phase was washed successively with water and brine, dried over sodium sulphate and filtered. The filtrate was concentrated under reduced pressure. This gave 40 g of a crude fraction of the title compound (VI-2), which was converted further directly to (VII-2) without further purification according to c).

### c) Methyl 6-[(cyclopropylcarbonyl)amino]-4-iodopyridine-2-carboxylate (VII-2)

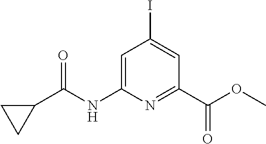 [SMILES: COC(=O)c1cc(I)cc(NC(=O)C2CC2)n1]

To a stirred solution of 40.0 g of the crude fraction of methyl 6-amino-4-bromopyridine-2-carboxylate (VI-2) prepared according to b) in 400 ml of dry dichloromethane were added 26 ml (322 mmol) of pyridine. After stirring at room temperature for ten minutes, the mixture was cooled in an ice bath, and 14.6 ml (161 mmol) of cyclopropanecarbonyl chloride were added. The reaction mixture was stirred at room temperature for a further 16 h. Subsequently, the mixture was diluted with water and the phases were separated. The aqueous phase was extracted repeatedly with dichloromethane. The combined organic phases were washed successively with water and brine, dried over sodium sulphate, filtered and concentrated under reduced pressure. The residue was purified by chromatography using a silica gel column (ethyl acetate in hexane, 0%-30%), followed by recrystallization from ethyl acetate:hexane (1:10, V/V). This gave 35.0 g (64.0% of theory over the three stages a to c) of the title compound (VII-2).

1H-NMR (400.0 MHz, d₆-DMSO): δ=11.32 (s, 1H), 8.77 (s, 1H), 8.04 (s, 1H), 3.87 (s, 3H), 2.07 (m, 1H), 0.83 (d, 4H)

### 6-[(Cyclopropylcarbonyl)amino]-4-iodopyridine-2-carboxylic acid (VIII-2)

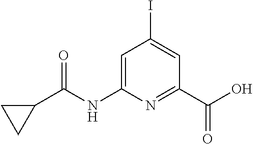 [SMILES: O=C(O)c1cc(I)cc(NC(=O)C2CC2)n1]

To a suspension of 23.0 g (66.5 mmol) of methyl 6-[(cyclopropylcarbonyl)amino]-4-iodopyridine-2-carboxylate (VII-2) in a mixture of 150 ml of THF and 150 ml of water at 25° C. were added 8.40 g (199 mmol) of lithium hydroxide monohydrate. The mixture was stirred at 25° C. for another 16 h. Subsequently, the THF was removed under reduced pressure and aqueous phase was adjusted to pH 3 by means of 6 N hydrochloric acid. The precipitating solids were filtered off and dried under reduced pressure. This gave 19.0 g (90.0% of theory) of the title compound (VIII-2).

1H-NMR (400.0 MHz, d₆-DMSO): δ=12.63 ppm (broad, 1H), 8.62 (s, 1H), 7.98 (s, 1H), 2.34 (m, 1H), 0.89 (d, 4H)

### N-(4-Cyanobenzyl)-6-[(cyclopropylcarbonyl)amino]-4-iodopyridine-2-carboxamide (IX-2)

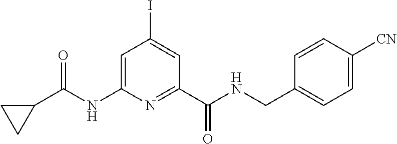 [SMILES: N#Cc1ccc(CNC(=O)c2cc(I)cc(NC(=O)C3CC3)n2)cc1]

To a stirred solution of 9.5 g (28.6 mmol) of 6-[(cyclopropylcarbonyl)amino]-4-iodopyridine-2-carboxylic acid (VIII-2) in 80 ml of dry dimethylformamide at room temperature were added 19.6 g (51.5 mmol) of HATU. After stirring at room temperature for a further hour, 5.3 g (31.5 mmol) of 4-(aminomethyl)benzonitrile hydrochloride and subsequently 14.0 ml (100 mmol) of triethylamine were added. The mixture was stirred at room temperature for another 16 h and then poured gradually into ice-water. The precipitate formed was filtered off, washed with diethyl ether and purified by chromatography using a silica gel column (eluent: ethyl acetate:dichloromethane:hexane 1:1:2). This gave 9.3 g (73.0% of theory) of the title compound (IX-2).

1H-NMR (400.0 MHz, CDCl₃): δ=8.82 (s, 1H), 8.29 ppm (s, 1H), 8.08 (m, broad, 2H), 7.63 (m, 2H), 7.44 (m, 2H), 4.69 (d, 2H), 1.53 (m, 1H), 1.13 (m, 2H), 0.95 (m, 2H)

### By Process B

### N-(4-Cyanobenzyl)-6-[(cyclopropylcarbonyl)amino]-4-[3-(pentafluoroethyl)-1H-pyrazol-1-yl]pyridine-2-carboxamide (I-188)

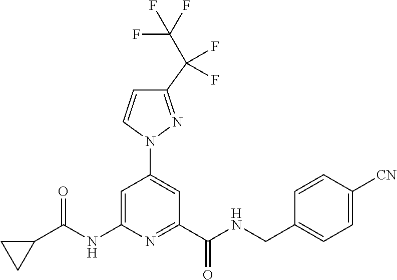 [SMILES: N#Cc1ccc(CNC(=O)c2cc(-n3ccc(C(F)(F)C(F)(F)F)n3)cc(NC(=O)C3CC3)n2)cc1]

120 mg (0.26 mmol) of N-(4-cyanobenzyl)-6-[(cyclopropylcarbonyl)amino]-4-iodopyridine-2-carboxamide (IX-2) were initially charged in 5 ml of degassed 1,4-dioxane under argon in a rolled flange vessel, and 70.0 mg (0.37 mmol) of 3-(pentafluoroethyl)-1H-pyrazole, 5.2 mg (0.02 mmol) of copper(I) iodide and 13.4 mg (0.08 mmol) of potassium iodide were added. The vial was flooded again with argon, and 7.7 mg (0.05 mmol) of trans-N,N′-dimethylcyclohexane-1,2-diamine (racemic) were added. The vessel was closed and heated to 130° C. in an aluminium block overnight. The filtrate was concentrated under reduced pressure and the residue was purified by means of MPLC separation (eluent: dichloromethane:ethyl acetate, 7:3), followed by a second separation (reversed phase, eluent:water:acetonitrile+0.1% formic acid). This gave 56 mg (96% purity, 39.6% of theory) of the title compound (1-188).

log P (neutral/acidic): 3.57/3.70; MH⁺: 505; 1H-NMR (400.0 MHz, d₆-DMSO): δ=11.092 (9.1); 9.085 (6.7); 9.081 (6.3); 9.078 (6.9); 8.963 (2.4); 8.947 (5.2); 8.931 (2.5); 8.784 (11.4); 8.779 (11.8); 8.314 (0.8); 8.213 (11.9); 8.208 (11.9); 7.842 (13.6); 7.821 (16.0); 7.561 (13.5); 7.540 (11.8); 7.162 (8.7); 7.155 (8.8); 4.662 (10.2); 4.646 (10.2); 3.320 (172.1); 2.945 (1.7); 2.892 (0.4); 2.785 (1.3); 2.732 (0.3); 2.676 (0.9); 2.672 (1.3); 2.667 (1.0); 2.525 (3.5); 2.512 (74.7); 2.507 (152.9); 2.503 (203.1); 2.498 (149.5); 2.494 (74.3); 2.334 (0.9); 2.330 (1.2); 2.325 (0.9); 2.075 (7.9); 2.062 (1.9); 2.057 (2.2); 2.052 (1.8); 2.045 (3.7); 2.032 (2.2); 2.027 (2.1); 2.013 (1.0); 1.958 (1.4); 0.917 (0.6); 0.894 (11.6); 0.885 (15.7); 0.866 (8.6); 0.845 (0.8); 0.146 (0.9); 0.008 (7.5); 0.000 (195.9); −0.008 (8.2); −0.150 (0.9)

### By Process C

### 2,6-Dichloro-4-[4-(trifluoromethoxy)phenyl]pyridine (XIV-1)

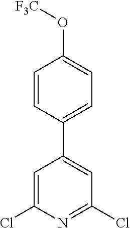 [SMILES: FC(F)(F)Oc1ccc(-c2cc(Cl)nc(Cl)c2)cc1]

Under argon, 15.0 g (54.7 mmol) of 2,6-dichloro-4-iodopyridine (XIII-1), 12.4 g (60.2 mmol) of [4-(trifluoromethoxy)phenyl]boric acid and 3.2 g (2.73 mmol) of tetrakis(triphenylphosphine)palladium(0) were added to 350 ml of 1,4-dioxane. Subsequently, 55 ml of a two-molar sodium carbonate solution were added and the mixture was stirred under reflux and under argon for 18 h. The cooled reaction mixture was then admixed with 200 ml of ethyl acetate and filtered through Celite. The filtrate was concentrated, taken up in dichloromethane and washed with water. Purification was effected by means of MPLC (eluent: cyclohexane:dichloromethane, 85:15). This gave 12.2 g (99% purity, 71.6% of theory) of the title compound (XIV-1).

log P (neutral/acidic): 4.62/4.63; MH⁺: 308; 1H-NMR (400.0 MHz, d₆-DMSO): δ=8.067 (0.4); 8.059 (4.4); 8.054 (1.4); 8.042 (1.4); 8.037 (5.0); 8.029 (0.5); 7.975 (16.0); 7.544 (2.6); 7.523 (2.4); 3.320 (33.3); 2.525 (0.4); 2.521 (0.6); 2.512 (9.7); 2.507 (20.9); 2.503 (29.5); 2.498 (21.8); 2.494 (10.3); 0.008 (0.3); 0.000 (11.5); −0.008 (0.4)

### 2-Chloro-4-[4-(trifluoromethoxy)phenyl]-6-vinylpyridine (XV-1)

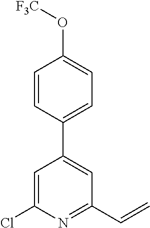 [SMILES: C=Cc1cc(-c2ccc(OC(F)(F)F)cc2)cc(Cl)n1]

Under argon, 9.0 g (29.2 mmol) of 2,6-dichloro-4-[4-(trifluoromethoxy)phenyl]pyridine (XIV-1) and 5.2 g (33.5 mmol) of 4,4,5,5-tetramethyl-2-vinyl-1,3,2-dioxaborolane were initially charged in 84 ml of 1,4-dioxane, and 37 ml of a two-molar sodium carbonate solution were added. Under argon, 6.8 g (5.84 mmol) of tetrakis(triphenylphosphine)palladium(0) were then added and the mixture was heated to 120° C. in an IR Discover microwave for 60 minutes. After cooling, the reaction mixture was diluted with methanol and filtered through Celite. The filtrate was concentrated and the residue was purified by means of MPLC (eluent: cyclohexane:dichloromethane, 4:1). This gave 3.6 g (97% purity, 40.0% of theory) of the title compound (XV-1).

log P (neutral/acidic): 4.72/4.77; MH⁺: 300; 1H-NMR (400.0 MHz, d₆-DMSO): δ=8.060 (0.4); 8.030 (14.9); 8.009 (16.0); 7.976 (0.9); 7.883 (15.1); 7.768 (15.8); 7.544 (12.7); 7.522 (11.9); 6.891 (4.0); 6.864 (4.5); 6.848 (5.1); 6.821 (5.1); 6.392 (8.1); 6.348 (6.8); 5.623 (7.7); 5.596 (7.3); 3.321 (131.5); 2.673 (0.9); 2.503 (158.4); 2.331 (0.9); 0.147 (0.4); 0.002 (73.0); 0.000 (78.1); −0.149 (0.4)

### 6-Chloro-4-[4-(trifluoromethoxy)phenyl]pyridine-2-carboxylic acid (XVI-1)

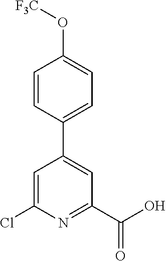 [SMILES: O=C(O)c1cc(-c2ccc(OC(F)(F)F)cc2)cc(Cl)n1]

To a solution of 3.6 g (12.0 mmol) of 2-chloro-4-[4-(trifluoromethoxy)phenyl]-6-vinylpyridine (XV-1) in 200 ml of acetone and 200 ml of water were cautiously added 9.5 g (60 mmol) of potassium permanganate. The mixture warmed up to about 40° C. and was then stirred for a further 3 h. After the reaction had ended, the mixture was filtered through a sintered glass filter. The acetone in the filtrate was removed under reduced pressure and the alkaline aqueous phase was then washed with ethyl acetate. Thereafter, the pH of the aqueous phase was adjusted to pH 4, the water phase was extracted with ethyl acetate, and the organic phase was concentrated. This gave 3.0 g (98% purity, 77.0% of theory) of the title compound (XVI-1).

log P (neutral/acidic): 1.17/3.00; MH⁺: 318; 1H-NMR (400.0 MHz, d₆-DMSO): δ=8.291 (10.7); 8.287 (11.4); 8.138 (11.4); 8.134 (11.1); 8.079 (1.4); 8.071 (12.4); 8.066 (4.1); 8.054 (4.2); 8.049 (13.8); 8.042 (1.5); 7.550 (8.2); 7.530 (7.5); 4.092 (1.0); 3.325 (15.8); 3.170 (16.0); 2.677 (0.5); 2.672 (0.6); 2.668 (0.5); 2.526 (1.6); 2.512 (36.7); 2.508 (76.9); 2.503 (107.3); 2.499 (80.3); 2.495 (38.7); 2.482 (2.1); 2.335 (0.5); 2.330 (0.6); 2.326 (0.5); 2.118 (2.7); 1.990 (0.4); 1.910 (8.6); 1.142 (5.8); 0.008 (1.1); 0.000 (31.7); −0.008 (1.1)

### 6-Chloro-N-(4-cyanobenzyl)-4-[4-(trifluoromethoxy)phenyl]pyridine-2-carboxamide (XVII-1)

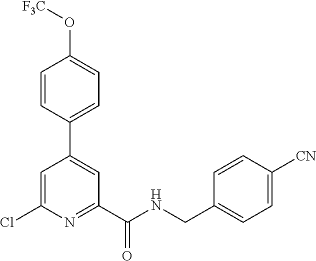 [SMILES: N#Cc1ccc(CNC(=O)c2cc(-c3ccc(OC(F)(F)F)cc3)cc(Cl)n2)cc1]

To a solution of 5.2 g (16.3 mmol) of 6-chloro-4-[4-(trifluoromethoxy)phenyl]pyridine-2-carboxylic acid (XVI-1) in 15 ml of toluene were cautiously added 1.3 ml (18.0 mmol) of thionyl chloride, dissolved in 5 ml of toluene. The mixture was heated under reflux for 2 h, cooled and then concentrated under reduced pressure. The residue was dissolved in 7 ml of acetonitrile and introduced into a solution of 2.6 g (19.6 mmol) of 4-(aminomethyl)benzonitrile and 6.6 g (65.4 mmol) of triethylamine in 15 ml of acetonitrile. This mixture was then heated under reflux overnight. After cooling, the mixture was concentrated under reduced pressure, the residue was admixed with water and dichloromethane, and the organic phase was removed. After drying and concentration, the latter was purified by means of MPLC using a silica gel cartridge (eluent: cyclohexane:ethyl acetate, 1:9). This gave 4.1 g (99% purity, 57.4% of theory) of the title compound (XVII-1).

log P (neutral/acidic): 4.19/4.23; MH⁺: 432; 1H-NMR (400.0 MHz, d₆-DMSO): δ=9.503 (2.4); 9.487 (5.0); 9.471 (2.4); 8.287 (12.1); 8.283 (12.9); 8.137 (12.9); 8.133 (12.4); 8.085 (0.8); 8.078 (1.5); 8.071 (14.0); 8.066 (4.8); 8.054 (4.8); 8.049 (15.6); 8.042 (1.7); 7.811 (13.4); 7.791 (16.0); 7.550 (9.9); 7.541 (14.9); 7.530 (9.8); 7.521 (12.7); 5.755 (1.6); 4.598 (9.8); 4.582 (9.9); 3.325 (196.7); 2.678 (0.6); 2.673 (0.8); 2.669 (0.6); 2.526 (2.1); 2.513 (44.6); 2.508 (91.9); 2.504 (126.9); 2.499 (95.6); 2.495 (47.0); 2.335 (0.5); 2.331 (0.7); 2.326 (0.6); 0.146 (0.7); 0.008 (5.2); 0.000 (147.0); −0.008 (5.7); −0.150 (0.7)

### N-(4-Cyanobenzyl)-6-(methylamino)-4-[4-(trifluoromethoxy)phenyl]pyridine-2-carboxamide (XVIII-1)

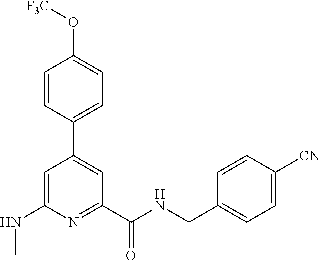 [SMILES: CNc1cc(-c2ccc(OC(F)(F)F)cc2)cc(C(=O)NCc2ccc(C#N)cc2)n1]

600 mg (1.4 mmol) of 6-chloro-N-(4-cyanobenzyl)-4-[4-(trifluoromethoxy)phenyl]pyridine-2-carboxamide (XVII-1) and 21 ml (41.6 mmol) of a two-molar solution of methanamine in THF were initially charged in a 300 ml autoclave, argon was injected to 10 bar and the mixture was stirred (800 rpm) at 140° C. for 75 h. After cooling, the mixture was concentrated and the residue was purified by means of MPLC using a silica gel cartridge (eluent: cyclohexane:ethyl acetate, 1:1). This gave 400 mg (98% purity, 66.2% of theory) of the title compound (XVIII-1).

log P (neutral/acidic): 3.63/3.63; MH⁺: 427; 1H-NMR (400.0 MHz, d₆-DMSO): δ=9.163 (1.6); 9.147 (3.4); 9.131 (1.6); 8.314 (0.7); 7.836 (1.1); 7.829 (9.7); 7.824 (3.6); 7.813 (12.2); 7.807 (12.7); 7.793 (11.8); 7.523 (9.7); 7.502 (15.4); 7.482 (6.3); 7.435 (8.6); 7.431 (9.0); 6.895 (8.4); 6.892 (8.5); 6.880 (1.0); 6.868 (2.4); 6.856 (2.4); 5.754 (2.8); 4.608 (6.8); 4.592 (6.9); 4.057 (1.2); 4.039 (3.7); 4.021 (3.8); 4.003 (1.3); 3.321 (164.4); 2.949 (15.3); 2.937 (15.4); 2.676 (0.6); 2.672 (0.9); 2.667 (0.7); 2.525 (2.3); 2.511 (50.4); 2.507 (103.9); 2.503 (143.4); 2.498 (107.9); 2.494 (52.7); 2.334 (0.6); 2.329 (0.9); 2.325 (0.6); 1.989 (16.0); 1.194 (4.2); 1.176 (8.3); 1.158 (4.1); 0.146 (0.7); 0.008 (5.6); 0.000 (161.2); −0.008 (6.0); −0.150 (0.7)

### N-(4-Cyanobenzyl)-6-[(cyclopropylcarbonyl)(methyl)amino]-4-[4-(trifluoromethoxy)phenyl]pyridine-2-carboxamide (I-215)

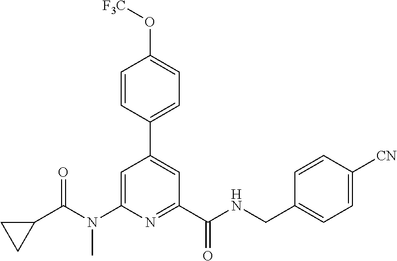 [SMILES: CN(C(=O)C1CC1)c1cc(-c2ccc(OC(F)(F)F)cc2)cc(C(=O)NCc2ccc(C#N)cc2)n1]

142 mg (0.33 mmol) of N-(4-cyanobenzyl)-6-(methylamino)-4-[4-(trifluoromethoxy)phenyl]pyridine-2-carboxamide (XVIII-1) were dissolved in 7 ml of absolute dichloromethane, and then 108 mg (0.83 mmol) of N,N-diisopropylethylamine and 38 mg (0.36 mmol) of cyclopropanecarbonyl chloride were added. The mixture was then stirred at room temperature overnight. Subsequently, 4 ml of water were added, and the organic phase was removed and concentrated. The residue was purified by means of MPLC using a silica gel cartridge (eluent: cyclohexane:ethyl acetate, 1:1). This gave 150 mg (98% purity, 89.3% of theory) of the title compound (1-215).

log P (neutral/acidic): 3.84/3.85; MH⁺: 495; 1H-NMR (400.0 MHz, d₆-DMSO): δ=9.424 (2.3); 9.408 (5.1); 9.392 (2.4); 8.315 (0.3); 8.183 (11.4); 8.179 (12.2); 8.055 (11.8); 8.051 (11.2); 8.026 (1.5); 8.018 (13.2); 8.013 (4.5); 8.001 (4.8); 7.996 (14.9); 7.989 (1.7); 7.816 (13.2); 7.795 (16.0); 7.554 (10.3); 7.548 (15.3); 7.532 (11.0); 7.527 (12.7); 5.755 (0.9); 4.629 (9.0); 4.613 (9.0); 3.487 (39.9); 3.320 (112.3); 3.178 (0.5); 3.164 (0.5); 2.677 (0.6); 2.672 (0.9); 2.668 (0.6); 2.526 (2.2); 2.512 (49.6); 2.508 (103.7); 2.503 (143.8); 2.498 (106.8); 2.494 (50.7); 2.334 (0.6); 2.330 (0.8); 2.325 (0.6); 1.843 (0.6); 1.831 (1.4); 1.823 (1.7); 1.812 (2.5); 1.802 (1.8); 1.793 (1.4); 1.781 (0.7); 1.233 (0.4); 0.939 (2.0); 0.929 (5.6); 0.921 (7.9); 0.910 (6.2); 0.903 (2.5); 0.796 (2.4); 0.788 (5.6); 0.780 (5.0); 0.769 (5.9); 0.760 (4.5); 0.751 (1.7); 0.146 (0.4); 0.008 (3.3); 0.000 (101.4); −0.009 (3.2); −0.150 (0.4)

### By Process F

### N-(4-Cyanobenzyl)-6-[(cyclopropylcarbonothioyl)amino]-4-[4-(trifluoromethoxy)phenyl]pyridine-2-carbothioamide (I-181)

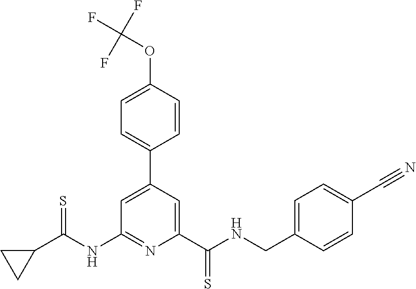 [SMILES: N#Cc1ccc(CNC(=S)c2cc(-c3ccc(OC(F)(F)F)cc3)cc(NC(=S)C3CC3)n2)cc1]

To an initial charge of 51 mg (0.11 mmol) of N-(4-cyanobenzyl)-6-[(cyclopropylcarbonyl)amino]-4-[4-(trifluoromethoxy)phenyl]pyridine-2-carboxamide (I-044) in toluene (3 ml) were added 86 mg (0.21 mmol) of 2,4-bis(4-methoxyphenyl)-1,3,2,4-dithiadiphosphetane 2,4-disulphide (Lawesson's reagent). The mixture was stirred at 100° C. for 2 h. Subsequently, the reaction mixture was cooled to room temperature and concentrated under reduced pressure, and the residue was purified by column chromatography purification using a cyclohexane/ethyl acetate gradient as eluent. This gave 18 mg (100% purity, 33% of theory) of the title compound (1-181).

log P (neutral/acidic): 5.43/5.54; MH⁺: 513; ¹H-NMR (400 MHz, D₆-DMSO) δ ppm: 1.03-1.08 (m, 2H), 1.17-1.23 (m, 2H), 2.50-2.55 (m, 1H), 5.13 (d, 2H), 7.56-7.58 (m, 4H), 7.82-7.84 (m, 2H), 7.91-7.94 (m, 2H), 8.50 (s, 1H), 8.89 (s, 1H), 11.19 (s, 1H), 12.37 (s, 1H).

### By Process G

### Methyl (4-cyanobenzyl)({6-[(cyclopropylcarbonyl)amino]-4-[4-(trifluoromethoxy)phenyl]pyridin-2-yl}carbonyl)carbamate (1-231)

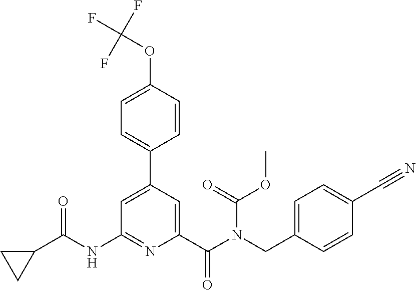 [SMILES: COC(=O)N(Cc1ccc(C#N)cc1)C(=O)c1cc(-c2ccc(OC(F)(F)F)cc2)cc(NC(=O)C2CC2)n1]

### Methyl (4-cyanobenzyl)({6-[(cyclopropylcarbonyl)(methoxycarbonyl)amino]-4-[4-(trifluoromethoxy)phenyl]pyridin-2-yl}carbonyl)carbamate (1-232)

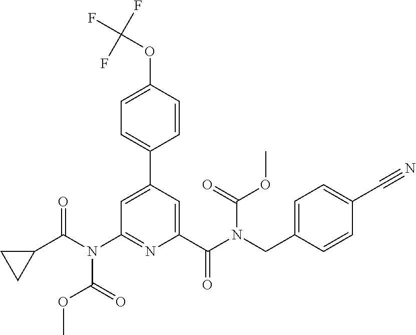 [SMILES: COC(=O)N(Cc1ccc(C#N)cc1)C(=O)c1cc(-c2ccc(OC(F)(F)F)cc2)cc(N(C(=O)OC)C(=O)C2CC2)n1]

### Methyl {6-[(4-cyanobenzyl)carbamoyl]-4-[4-(trifluoromethoxy)phenyl]pyridin-2-yl}(cyclopropylcarbonyl)carbamate (1-233)

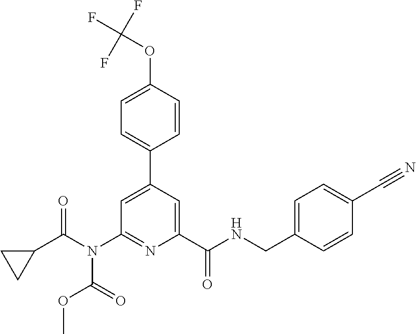 [SMILES: COC(=O)N(C(=O)C1CC1)c1cc(-c2ccc(OC(F)(F)F)cc2)cc(C(=O)NCc2ccc(C#N)cc2)n1]

166 mg (0.35 mmol) of N-(4-cyanobenzyl)-6-[(cyclopropylcarbonyl)amino]-4-[4-(trifluoromethoxy)phenyl]pyridine-2-carboxamide (I-044) were dissolved in DMF (4 ml), and 55 mg (1.38 mmol, 60% purity) of sodium hydride were added at 0° C. The mixture was stirred at room temperature for 15 min. Subsequently, 65 mg (0.69 mmol) of methyl chloroformate were added, and the mixture was stirred at room temperature for 14 h. Water was then added, and the mixture was extracted three times with ethyl acetate. The combined organic phases were washed with saturated NaCl solution, dried over magnesium sulphate and filtered. The solvent was removed under reduced pressure and the residue was purified by column chromatography purification using a cyclohexane/ethyl acetate gradient as eluent. This gave 8 mg (68% purity, 3% of theory) of the title compound (1-231), 63 mg (93% purity, 29% of theory) of the title compound (1-232) and 23 mg (90% purity, 11% of theory) of the title compound (1-233).

(I-231): log P (neutral/acidic): 4.43/4.41; MH⁺: 539; ¹H-NMR (400 MHz, D₆-DMSO) δ ppm: 0.85-0.87 (m, 4H), 2.09-2.12 (m, 1H), 3.54 (s, 3H), 5.06 (s, 2H), 7.52-7.63 (m, 4H), 7.76 (d, 1H), 7.86-7.94 (m, 4H), 8.56 (d, 1H), 11.04 (s, 1H).

(I-232): log P (neutral/acidic): 4.31/4.30; MH⁺: 597; ¹H-NMR (400 MHz, D₆-DMSO) δ ppm: 0.97-1.07 (m, 4H), 2.77-2.81 (m, 1H), 3.45 (s, 3H), 3.73 (s, 3H), 5.07 (s, 2H), 7.54-7.57 (m, 4H), 7.82-7.84 (m, 2H), 8.07-8.11 (m, 3H), 8.20 (d, 1H).

(I-233): log P (neutral/acidic): 3.91/3.90; MH⁺: 539; ¹H-NMR (400 MHz, D₆-DMSO) δ ppm: 1.00-1.06 (m, 4H), 2.81-2.84 (m, 1H), 3.73 (s, 3H), 4.57 (d, 2H), 7.51-7.55 (m, 4H), 7.79-7.82 (m, 2H), 8.02-8.06 (m, 2H), 8.11 (d, 1H), 8.35 (d, 1H), 9.43 (t, 1H).

### By Process H

### 2-[3-Fluoro-4-(pentafluoroethyl)phenyl]-4,4,5,5-tetramethyl-1,3,2-dioxaborolane

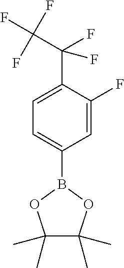 [SMILES: CC1(C)OB(c2ccc(C(F)(F)C(F)(F)F)c(F)c2)OC1(C)C]

Under argon, 2.64 g (10.4 mmol) of bis(pinacolato)diboron were taken up in 50 ml of degassed dioxane, and 2.90 g (9.90 mmol) of 4-bromo-2-fluoro-1-(pentafluoroethyl)benzene, 0.81 g (0.99 mmol) of [1,1′-bis(diphenylphosphino)ferrocene]palladium(II) dichloride and 2.91 g (29.7 mmol) of potassium acetate were added. Subsequently, the mixture was stirred at 80° C. overnight. After cooling, the reaction mixture was freed of solvent, then the residue was taken up in acetonitrile and filtered through silica gel. The silica gel layer was washed with acetonitrile and dichloromethane, and the organic phases were combined and concentrated under reduced pressure. This gave 3.10 g (92.0% of theory) of the title compound.

¹H-NMR (400 MHz, D₆-DMSO) δ ppm: 7.91 (dd, 1H), 7.77-7.71 (m, 1H), 7.61 (d, 1H), 1.32 (s, 12H)

### 2,6-Dichloro-4-[3-fluoro-4-(pentafluoroethyl)phenyl]pyridine (XIV-2)

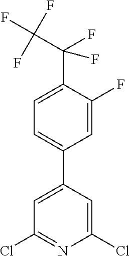 [SMILES: Fc1cc(-c2cc(Cl)nc(Cl)c2)ccc1C(F)(F)C(F)(F)F]

To an initial charge of 2.30 g (8.40 mmol) of 2,6-dichloro-4-iodopyridine (XIII-1) and 3.00 g (8.82 mmol) of 2-[3-fluoro-4-(pentafluoroethyl)phenyl]-4,4,5,5-tetramethyl-1,3,2-dioxaborolane in degassed dioxane (30 ml) under argon were added 8.4 ml of a 2M sodium carbonate solution. Subsequently, 97 mg (0.08 mmol) of tetrakis(triphenylphosphine)palladium(0) were added thereto, and the reaction mixture was stirred at 115° C. overnight. The cooled reaction mixture was freed of solvent under reduced pressure, then taken up in acetonitrile and filtered through silica gel. The silica gel was washed with acetonitrile. The filtrates were combined and the solvent was removed under reduced pressure. Purification was effected by means of MPLC using an RP-18 cartridge with an acetonitrile/water/0.1% formic acid gradient. This gave 2.13 g (93% purity, 65.5% of theory) of the title compound (XIV-2).

log P (neutral/acidic): 4.97/4.92; MH⁺: 360; ¹H-NMR (400 MHz, D₆-DMSO) δ ppm: 8.16 (d, 1H), 8.09 (s, 2H), 8.01 (d, 1H), 7.91 (dd, 1H)

### 6-Chloro-4-[3-fluoro-4-(pentafluoroethyl)phenyl]pyridin-2-amine (XXI-1)

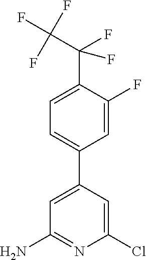 [SMILES: Nc1cc(-c2ccc(C(F)(F)C(F)(F)F)c(F)c2)cc(Cl)n1]

To an initial charge of 2.10 g (5.83 mmol) of 2,6-dichloro-4-[3-fluoro-4-(pentafluoroethyl)phenyl]pyridine (XIV-2) in dioxane (30 ml) were added 30 ml of a 35% aqueous ammonia solution. The autoclave vessel was purged with argon and the reaction was stirred therein at 150° C. under an argon pressure of 10 bar for 16 hours. After the addition of another 30 ml of ammonia solution, the reaction mixture was stirred at 150° C. under an argon pressure of 10 bar overnight. After cooling, the solvent was removed under reduced pressure. The residue was stirred in acetonitrile and the insoluble constituents were removed by filtration. The filtrate was freed of solvent under reduced pressure, before being purified by means of column chromatography using RP-18. It was possible to isolate two fractions comprising the title compound (XXI-1): 850 mg (92% purity, 39% of theory) and 497 mg (88% purity, 22% of theory).

log P (neutral/acidic): 3.83/3.85; MH⁺: 341; ¹H-NMR (400 MHz, D₆-DMSO) δ ppm: 7.87-7.83 (m, 2H), 7.74-7.72 (m, 1H), 6.94 (d, 1H), 6.70 (d, 1H), 6.61 (bs, 2H)

### N-{6-Chloro-4-[3-fluoro-4-(pentafluoroethyl)phenyl]pyridin-2-yl}cyclopropanecarboxamide (XXII-1)

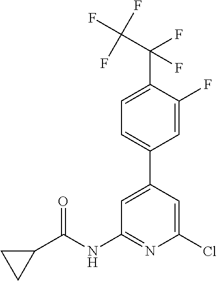 [SMILES: O=C(Nc1cc(-c2ccc(C(F)(F)C(F)(F)F)c(F)c2)cc(Cl)n1)C1CC1]

To an initial charge of 800 mg (2.11 mmol) of 6-chloro-4-[3-fluoro-4-(pentafluoroethyl)phenyl]pyridin-2-amine (XXI-1) in dichloromethane (35 ml) were added 1.15 ml (8.22 mmol) of triethylamine. After 10 min at room temperature, 0.26 ml (2.82 mmol) of cyclopropanecarbonyl chloride dissolved in dichloromethane (5 ml) was added dropwise. The reaction mixture was stirred at room temperature overnight. Subsequently, the solvent was removed under reduced pressure and the residue was taken up in toluene (15 ml). After the addition of 0.38 ml (4.70 mmol) of pyridine and 0.26 ml (2.82 mmol) of cyclopropanecarbonyl chloride, the mixture was stirred at 100° C. for 5 h. After cooling, the solvent was removed under reduced pressure. The residue was taken up in dichloromethane and washed repeatedly with saturated sodium chloride solution. The organic phase was separated, dried over sodium sulphate, filtered and freed of solvent under reduced pressure. 1.02 g (96% purity, quantitative) of the title compound (XXII-1) were obtained.

log P (neutral/acidic): 4.64/4.76; MH⁺: 409; ¹H-NMR (400 MHz, D₆-DMSO) δ ppm: 12.04+11.30 (2xs, 1H), 8.42 (d, 1H), 7.98-7.88 (m, 2H), 7.82-7.80 (m, 1H), 7.66 (s, 1H), 2.05-1.99 (m, 1H), 0.87-0.85 (m, 4H)

### 6-[(Cyclopropylcarbonyl)amino]-4-[3-fluoro-4-(pentafluoroethyl)phenyl]pyridine-2-carboxamide (XXIII-1)

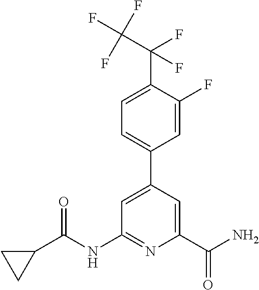 [SMILES: NC(=O)c1cc(-c2ccc(C(F)(F)C(F)(F)F)c(F)c2)cc(NC(=O)C2CC2)n1]

900 mg (2.11 mmol) of N-{6-chloro-4-[3-fluoro-4-(pentafluoroethyl)phenyl]pyridin-2-yl}cyclopropanecarboxamide (XXII-1) and 260 g (3.17 mmol) of sodium acetate were initially charged in methanol (20 ml) in a 50 ml autoclave vessel. Then 167 mg (0.21 mmol) of dichloro[(bisdiphenylphosphino)ferrocenyl]palladium(II) acetone adducts were added thereto and the vessel was purged with 10 bar argon. The second conduit in the autoclave contained ammonia and was additionally filled with CO. The reaction mixture was kept at 80° C. under 10 bar CO for 15 hours. After cooling, the solvent was removed under reduced pressure. The residue was suspended in acetonitrile, and insoluble constituents were removed by filtration. The resulting filtrate was freed of the solvent under reduced pressure. This gave 1.07 g (86% of theory, quantitative) of the title compound (XXIII-1).

log P (neutral/acidic): 3.17/3.18; MH⁺: 418; ¹H-NMR (400 MHz, D₆-DMSO) δ ppm: 12.05 (bs, 1H), 11.06 (s, 1H), 8.56 (s, 1H), 8.03 (s, 1H), 7.99-7.83 (m, 3H), 5.40 (bs, 1H), 2.08-2.02 (m, 1H), 0.89-0.87 (m, 4H)

### N-(Cyclopropylcarbonyl)-6-[(cyclopropylcarbonyl)amino]-4-[3-fluoro-4-(pentafluoroethyl)-phenyl]pyridine-2-carboxamide (I-264)

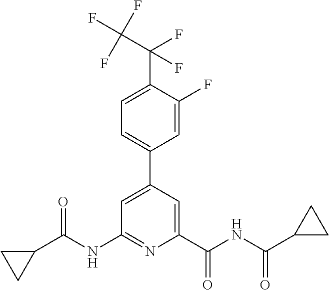 [SMILES: O=C(NC(=O)C1CC1)c1cc(-c2ccc(C(F)(F)C(F)(F)F)c(F)c2)cc(NC(=O)C2CC2)n1]

100 mg (0.21 mmol) of 6-[(cyclopropylcarbonyl)amino]-4-[3-fluoro-4-(pentafluoroethyl)phenyl]pyridine-2-carboxamide (XXIII-1) were dissolved in dry tetrahydrofuran (5 ml), and 7.5 mg (0.31 mmol) of sodium hydride were added at room temperature. The mixture was stirred at room temperature for 15 min. Subsequently, 22 mg (0.21 mmol) of cyclopropanecarbonyl chloride were added and the mixture was stirred at 40° C. for 2 h. After cooling, the solvent was removed under reduced pressure and the residue was purified by column chromatography purification using RP-18 with an acetonitrile/water gradient as eluent. This gave 38 mg (100% purity, 38% of theory) of the title compound (1-264).

log P (neutral/acidic): 4.53/4.62; MH⁺: 486; ¹H-NMR (400 MHz, D₆-DMSO) δ ppm: 11.28 (s, 1H), 10.72 (s, 1H), 8.68 (d, 1H), 8.14 (d, 1H), 8.02 (d, 1H), 7.94-7.85 (m, 2H), 2.82-2.76 (m, 1H), 2.08-1.99 (m, 1H), 1.07-0.99 (m, 4H), 0.91-0.89 (m, 4H)

### Methyl 6-[(cyclopropylcarbonyl)amino]-4-[4-(trifluoromethoxy)phenyl]pyridine-2-carboxylate (XXIII-2)

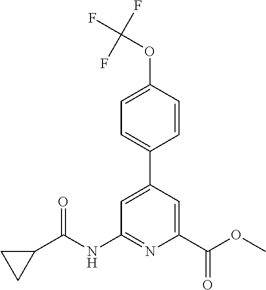 [SMILES: COC(=O)c1cc(-c2ccc(OC(F)(F)F)cc2)cc(NC(=O)C2CC2)n1]

5.43 g (15.2 mmol) of N-{6-chloro-4-[4-(trifluoromethoxy)phenyl]pyridin-2-yl}cyclopropanecarboxamide (XXII-2; synthesis analogous to XXII-1) and 1.87 g (22.8 mmol) of sodium acetate were initially charged in methanol (137 ml) in a 300 ml autoclave vessel. Then 601 mg (0.76 mmol) of dichloro[(bisdiphenylphosphino)ferrocenyl]palladium(II) acetone adducts were added. The reaction mixture was carbonylated at 80° C. under 10 bar CO for 15 hours. After cooling to room temperature, the mixture was admixed with saturated sodium chloride solution and extracted repeatedly with ethyl acetate. The combined organic phases were dried over magnesium sulphate and filtered. The solvent was removed under reduced pressure. This gave 5.75 g (100% purity, 99% of theory) of the title compound (XXIII-2).

log P (neutral/acidic): 3.54/3.63; MH⁺: 381; ¹H-NMR (400 MHz, D₆-DMSO) δ ppm: 11.33 (s, 1H), 8.64 (d, 1H), 8.02 (d, 1H), 7.88-7.92 (m, 2H), 7.52-7.54 (m, 2H), 3.92 (s, 3H), 2.10-2.14 (m, H), 0.84-0.86 (m, 4H)

### 6-[(Cyclopropylcarbonyl)amino]-4-[4-(trifluoromethoxy)phenyl]pyridine-2-carboxylic acid

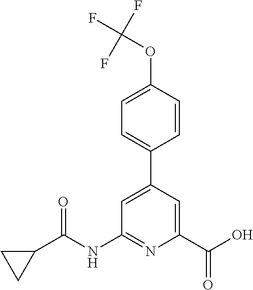 [SMILES: O=C(O)c1cc(-c2ccc(OC(F)(F)F)cc2)cc(NC(=O)C2CC2)n1]

4.00 g (10.5 mmol) of methyl 6-[(cyclopropylcarbonyl)amino]-4-[4-(trifluoromethoxy)phenyl]pyridine-2-carboxylate (XXIII-2) were dissolved in methanol (105 ml). Then 14.1 ml (14.1 mmol) of 1 N sodium hydroxide solution were added. The reaction mixture was stirred at room temperature overnight and adjusted to pH=3 with 1 N hydrochloric acid. The methanol was removed under reduced pressure. The precipitated solids were filtered off with suction, washed with water and dried. This gave 2.68 g (88% purity, 62% of theory) of the title compound.

log P (neutral/acidic): 1.25/2.72; MH⁺: 367; ¹H-NMR (400 MHz, D₆-DMSO) δ ppm: 13.37 (s, 1H), 11.26 (s, 1H), 8.60 (d, 1H), 8.00 (d, 1H), 7.89-7.91 (m, 2H), 7.49-7.54 (m, 2H), 2.08-2.15 (m, 1H), 0.84-0.86 (m, 4H)

### 6-[(Cyclopropylcarbonyl)amino]-N-isopropoxy-4-[4-(trifluoromethoxy)phenyl]pyridine-2-carboxamide (I-237)

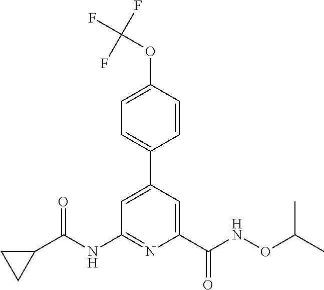 [SMILES: CC(C)ONC(=O)c1cc(-c2ccc(OC(F)(F)F)cc2)cc(NC(=O)C2CC2)n1]

To an initial charge of 150 mg (0.41 mmol) of 6-[(cyclopropylcarbonyl)amino]-4-[4-(trifluoromethoxy)phenyl]pyridine-2-carboxylic acid in dioxane (7 ml) were added 94.2 mg (0.49 mmol) of EDC hydrochloride. The mixture was stirred at room temperature for one hour. Then 45.7 mg (0.41 mmol) of 2-(aminooxy)propane hydrochloride were added and the mixture was stirred at room temperature overnight. The mixture was concentrated and water was added. Then the mixture was extracted with dichloromethane, and the combined organic phases were dried over magnesium sulphate and filtered. The solvent was distilled off under reduced pressure and the residue was purified by column chromatography purification using a cyclohexane/ethyl acetate gradient as eluent. This gave 42.7 mg (100% purity, 25% of theory) of the title compound (I-237).

log P (neutral/acidic): 3.45/3.50; MH⁺: 424; ¹H-NMR (400 MHz, D₆-DMSO) δ ppm: 11.23 (s, 1H), 10.93 (s, 1H), 8.50 (d, 1H), 7.88-7.92 (m, 2H), 7.83 (d, 1H), 7.53-7.55 (m, 2H), 4.15-4.21 (m, 1H), 2.06-2.09 (m, 1H), 1.23 (d, 6H), 0.85-0.87 (m, 4H)

### By Process I

### N-{6-(Hydrazinocarbonyl)-4-[4-(trifluoromethoxy)phenyl]pyridin-2-yl}cyclopropanecarboxamide (XXIV-1)

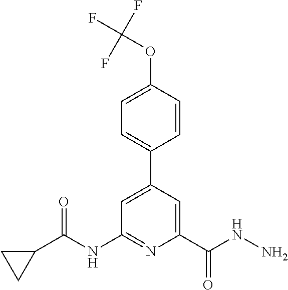 [SMILES: NNC(=O)c1cc(-c2ccc(OC(F)(F)F)cc2)cc(NC(=O)C2CC2)n1]

1.50 g (3.94 mmol) of methyl 6-[(cyclopropylcarbonyl)amino]-4-[4-(trifluoromethoxy)phenyl]pyridine-2-carboxylate (XXIII-2) were dissolved in dioxane (90 ml). Then 1.10 ml (7.88 mmol, 35% by weight in water) of hydrazine hydrate were slowly added dropwise. The reaction mixture was first stirred at room temperature for two hours, then heated to boiling for two hours, cooled to room temperature, and finally concentrated to dryness under reduced pressure. This gave 2 g of the title compound as a crude product (76% purity), which was used in the next stage without further purification.

log P (neutral/acidic): 2.55/2.57; MH⁺: 381

### N-(6-{[(2E)-2-(4-Cyanobenzylidene)hydrazino]carbonyl}-4-[4-(trifluoromethoxy)phenyl] pyridin-2-yl)cyclopropanecarboxamide (I-252)

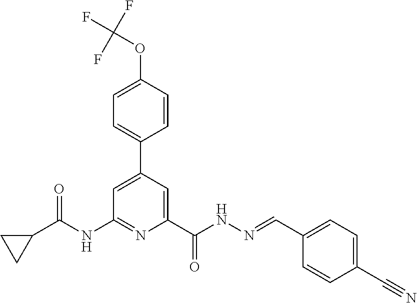 [SMILES: N#Cc1ccc(/C=N/NC(=O)c2cc(-c3ccc(OC(F)(F)F)cc3)cc(NC(=O)C3CC3)n2)cc1]

To an initial charge of 50 mg (0.13 mmol) of N-{6-(hydrazinocarbonyl)-4-[4-(trifluoromethoxy)phenyl]pyridin-2-yl}cyclopropanecarboxamide (XXIV-1) in water (10 ml) were added two drops of concentrated sulphuric acid, and the mixture was heated to 65° C. Then 57 mg (0.43 mmol) of 4-cyanobenzaldehyde, dissolved in ethanol (10 ml), were added. The reaction mixture was stirred at 74° C. for 48 hours, cooled to room temperature, poured onto ice-water and neutralized with 10% sodium carbonate solution. The precipitated crystals were filtered off with suction and purified further by column chromatography purification with a cyclohexane/ethyl acetate gradient as eluent. This gave 18 mg (94% purity, 26% of theory) of the title compound (1-252).

log P (neutral/acidic): 3.83/3.88; MH⁺: 494; ¹H-NMR (400 MHz, D₆-DMSO) δ ppm: 11.95 (s, 1H), 11.07 (s, 1H), 8.55-8.57 (m, 2H), 8.01 (d, 1H), 7.92-7.95 (m, 6H), 7.54-7.56 (m, 2H), 2.07-2.10 (m, 1H), 0.85-0.89 (m, 4H)

### By Process J

### 2-Chloro-4-[4-(trifluoromethoxy)phenyl]pyridine 1-oxide (XXVII-1)

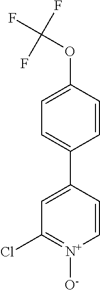 [SMILES: [O-][n+]1ccc(-c2ccc(OC(F)(F)F)cc2)cc1Cl]

To an initial charge of 30 g (109 mmol) of 2-chloro-4-[4-(trifluoromethoxy)phenyl]pyridine (XXVI-1, synthesis analogous to XIV-1 and XIV-2 from XIII-1) in dichloromethane (500 ml) were added 56.9 g (329 mmol) of 3-chloroperbenzoic acid (mCPBA). The mixture was stirred at 50° C. overnight, cooled to room temperature and washed with saturated sodium hydrogencarbonate solution. The solvent was removed under reduced pressure and the residue was purified by column chromatography purification with a dichloromethane/methanol gradient as eluent. This gave 20 g (63% of theory) of the title compound (XXVII-1).

MH⁺: 290

### 6-Chloro-4-[4-(trifluoromethoxy)phenyl]pyridine-2-carbonitrile (XXVIII-1)

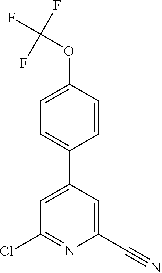 [SMILES: N#Cc1cc(-c2ccc(OC(F)(F)F)cc2)cc(Cl)n1]

To an initial charge of 20 g (69.1 mmol) of 2-chloro-4-[4-(trifluoromethoxy)phenyl]pyridine 1-oxide (XXVII-1) in acetonitrile (300 ml) were added 41.1 g (414 mmol) of trimethylsilyl cyanide (TMSCN) and 14 g (138 mmol) of triethylamine. The mixture was stirred at 100° C. overnight and cooled to room temperature. The volatile constituents were removed under reduced pressure and the residue was purified by column chromatography purification with an ethyl acetate/petroleum ether gradient as eluent. This gave 14 g (68% of theory) of the title compound (XXVIII-1).

MH⁺: 299; ¹H-NMR (300 MHz, CDCl₃) δ ppm: 7.82 (s, 1H), 7.74 (s, 1H), 7.66-7.70 (m, 2H), 7.40-7.42 (m, 2H).

### N-{6-Cyano-4-[4-(trifluoromethoxy)phenyl]pyridin-2-yl}cyclopropanecarboxamide (XXIX-1)

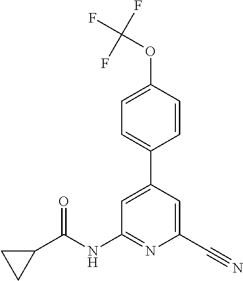 [SMILES: N#Cc1cc(-c2ccc(OC(F)(F)F)cc2)cc(NC(=O)C2CC2)n1]

A mixture of 5 g (16.7 mmol) of 6-chloro-4-[4-(trifluoromethoxy)phenyl]pyridine-2-carbonitrile (XXVIII-1), 2.85 g (33.5 mmol) of cyclopropanecarboxamide, 2 g (3.46 mmol) of XantPhos, 16.4 g (50.3 mmol) of caesium carbonate and 1 g (1.09 mmol) of Pd₂(dba)₃ in dioxane (200 ml) was stirred under a nitrogen atmosphere at 100° C. overnight and then cooled to room temperature. The solid constituents were filtered off and the filtrate was concentrated under reduced pressure. The residue was purified by column chromatography with an ethyl acetate/petroleum ether gradient as eluent. This gave 3.15 g (54% of theory, 97% purity) of the title compound (XXIX-1).

log P (neutral/acidic): 3.89/3.85; MH⁺: 348; ¹H-NMR (400 MHz, D₆-DMSO) δ ppm: 11.38 (s, 1H), 8.68 (d, 1H), 8.14 (d, 1H), 7.91-7.95 (m, 2H), 7.54-7.56 (m, 2H), 1.99-2.06 (m, 1H), 0.87-0.89 (m, 4H).

### N-{6-[N-(4-Cyanobenzyl)carbamimidoyl]-4-[4-(trifluoromethoxy)phenyl]pyridin-2-yl}cyclopropanecarboxamide (I-258)

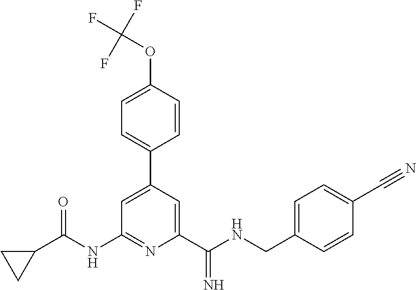 [SMILES: N#Cc1ccc(CNC(=N)c2cc(-c3ccc(OC(F)(F)F)cc3)cc(NC(=O)C3CC3)n2)cc1]

A mixture of 150 mg (0.43 mmol) of N-{6-cyano-4-[4-(trifluoromethoxy)phenyl]pyridin-2-yl}cyclopropanecarboxamide (XXIX-1) and 57 mg (0.43 mmol) of 4-(aminomethyl)benzonitrile in dioxane (0.5 ml) was heated to 90° C., 0.07 ml (0.51 mmol) of tin(IV) tetrachloride was added and the mixture was stirred at 95° C. for four hours. After cooling to room temperature, the mixture was admixed with saturated sodium hydrogencarbonate solution and dichloromethane. The organic phase was separated off and the solvent was removed under reduced pressure. A further reaction of identical batch size was conducted by the method described above. The two crude products were purified together by column chromatography purification with a cyclohexane/acetone/methanol gradient as eluent. This gave 7.3 mg (2% of theory) of the title compound (1-258).

log P (neutral/acidic): 4.24/2.08; MH⁺: 480; ¹H-NMR (400 MHz, D₆-DMSO) δ ppm: 11.07 (s, 1H), 9.27 (bs, 1H), 8.58 (s, 1H), 8.21 (s, 1H), 7.93-7.95 (m, 2H), 7.87-7.89 (m, 2H), 7.64-7.66 (m, 2H), 7.55-7.57 (m, 2H), 7.37 (bs, 1H), 4.77 (s, 2H) 2.04-2.10 (m, 1H), 0.85-0.89 (m, 4H).

In analogy to the examples and according to the above-described preparation methods, it is possible to obtain the following compounds of the formula (I), where the substituents R¹, R², R³, R⁴, R⁵, R⁶, Q¹, V¹ and V² have the definitions given in the following Table 1:

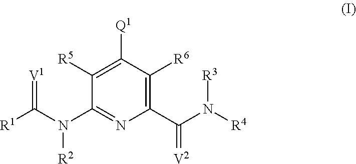

TABLE 1

| Ex. | R1 | R2 | R3 | R4 |
|---|---|---|---|---|
| I-001 | cyclopropyl | H | H | 3-cyanobenzyl |
| I-002 | cyclopropyl | H | H | 4-cyanobenzyl |
| I-003 | cyclopropyl | H | H | 3-fluorobenzyl |
| I-004 | cyclopropyl | H | H | 3-cyanobenzyl |
| I-005 | cyclopropyl | H | H | 4-cyanobenzyl |
| I-006 | cyclopropyl | H | H | 3-chlorobenzyl |
| I-007 | cyclopropyl | H | H | pyridin-2-ylmethyl |
| I-008 | cyclopropyl | H | H | 4-chlorobenzyl |
| I-009 | cyclopropyl | H | H | 2,2,2-trifluoroethyl |
| I-010 | cyclopropyl | H | H | 2,4-dichlorobenzyl |
| I-011 | cyclopropyl | H | H | (6-chloropyridin-3-yl)methyl |
| I-012 | cyclopropyl | H | H | cyclopropylmethyl |
| I-013 | cyclopropyl | H | H | cyclopropyl |
| I-014 | isopropyl | H | H | (6-chloropyridin-3-yl)methyl |
| I-015 | isopropyl | H | H | 4-cyanobenyl |
| I-016 | cyclopropyl | H | H | ethyl |
| I-017 | methyl | H | H | 4-cyanobenzyl |
| I-018 | cyclopropyl | H | H | isopropyl |
| I-019 | cyclopropyl | H | H | 4-cyanophenyl |
| I-020 | cyclopropyl | H | H | 4-cyanobenzyl |
| I-021 | cyclopropyl | H | H | 4-chlorophenyl |
| I-022 | cyclopropyl | H | H | 4-cyanophenyl |
| I-023 | 4-chlorophenyl | H | H | 4-cyanobenzyl |
| I-024 | cyclopropyl | H | H | (6-chloropyridin-3-yl)methyl |
| I-025 | cyclopropyl | H | H | cyanomethyl |
| I-026 | cyclopropyl | H | H | 4-cyano-3-fluorobenzyl |
| I-027 | cyclopropyl | H | H | 4-(trifluoromethyl)benzyl |
| I-028 | cyclopropyl | cyclopropylcarbonyl | H | 4-cyano-3-fluorobenzyl |
| I-029 | cyclopropyl | H | H | 4-cyanobenzyl |
| I-030 | cyclopropyl | H | H | cyclopropyl |
| I-031 | cyclopropyl | H | H | 1-cyclopropylethyl |
| I-032 | cyclopropyl | H | H | isobutyl |
| I-033 | cyclopropyl | H | H | cyclobutyl |
| I-034 | cyclopropyl | H | Methyl | pyridin-3-yl |
| I-035 | cyclopropyl | H | H | cyclopropyl |
| I-036 | cyclopropyl | H | Methyl | cyclopropyl |
| I-037 | cyclopropyl | H | H | n-butyl |
| I-038 | cyclopropyl | H | H | prop-2-yn-1-yl |
| I-039 | cyclopropyl | cyclopropylcarbonyl | H | 1-(4-cyanophenyl)ethyl |
| I-040 | cyclopropyl | H | H | 1-(4-cyanophenyl)ethyl |
| I-041 | cyclopropyl | H | H | 1-cyanocyclopropyl |
| I-042 | cyclopropyl | H | H | (6-cyanopyridin-3-yl)methyl |
| I-043 | cyclopropyl | H | Methyl | 4-cyanobenzyl |
| I-044 | cyclopropyl | H | H | 4-cyanobenzyl |
| I-045 | cyclobutyl | H | H | 4-cyanobenzyl |
| I-046 | cyclopropyl | H | H | 2-(4-cyanophenyl)ethyl |
| I-047 | 2-chlorophenyl | H | H | 4-cyanobenzyl |
| I-048 | ethyl | H | H | 4-cyanobenzyl |
| I-049 | cyclopropyl | H | H | cyclobutylmethyl |
| I-050 | cyclopropyl | H | H | 4-cyanobenzyl |
| I-051 | cyclopropyl | H | H | (6-cyanopyridin-3-yl)methyl |
| I-052 | cyclopropyl | H | H | 4-cyanobenzyl |
| I-053 | cyclopropyl | H | H | 4-cyanobenzyl |
| I-054 | cyclopropyl | H | H | 2-fluoro-4-(trifluoromethyl)benzyl |
| I-055 | cyclopropylmethyl | H | H | 4-cyanobenzyl |
| I-056 | cyclopropyl | H | H | 4-chloro-3-fluorobenzyl |
| I-057 | cyclopropyl | H | H | 1-[4-(trifluoromethyl)phenyl]ethyl |
| I-058 | cyclopropyl | H | H | 3-[4-(trifluoromethyl)phenyl]propyl |
| I-059 | cyclopropyl | H | H | 2,4-dichloro-5-fluorobenzyl |
| I-060 | cyclopropyl | H | H | (1S)-1-[4-(trifluoromethyl)phenyl]ethyl |
| I-061 | cyclopropyl | H | H | (1S)-1-[4-(trifluoromethyl)phenyl]ethyl |
| I-062 | cyclopropyl | H | H | 4-chloro-3-fluorobenzyl |
| I-063 | cyclopropyl | H | H | 2,4-dichloro-5-fluorobenzyl |
| I-064 | cyclopropyl | H | H | 3-fluoro-4-(trifluoromethyl)benzyl |
| I-065 | cyclopropyl | H | H | 2-fluoro-4-(trifluoromethyl)benzyl |
| I-066 | cyclopropyl | H | H | 4-chloro-2-fluorobenzyl |
| I-067 | cyclopropyl | H | H | 4-chloro-2-fluorobenzyl |
| I-068 | cyclopropyl | H | H | 4-cyano-2,6-difluorobenzyl |
| I-069 | isopropyl | H | H | 4-cyano-2,6-difluorobenzyl |
| I-070 | isopropyl | H | H | 4-cyano-2,6-difluorobenzyl |
| I-071 | isopropyl | H | H | 4-cyano-2,5-difluorobenzyl |
| I-072 | isopropyl | H | H | 4-cyano-3,5-difluorobenzyl |
| I-073 | isopropyl | H | H | 4-cyano-3,5-difluorobenzyl |
| I-074 | cyclopropyl | H | H | 4-cyano-2,5-difluorobenzyl |
| I-075 | isopropyl | H | H | 1-cyanocyclopropyl |
| I-076 | cyclopropyl | H | H | 1-cyanocyclopropyl |
| I-077 | cyclopropyl | H | H | 4-cyano-2-fluorobenzyl |
| I-078 | isopropyl | H | H | 4-cyano-2-fluorobenzyl |
| I-079 | cyclopropyl | H | H | 4-cyano-2-fluorobenzyl |
| I-080 | cyclopropyl | H | H | 4-cyano-3-fluorobenzyl |
| I-081 | isopropyl | H | H | 4-cyano-2-fluorobenzyl |
| I-082 | cyclopropyl | H | H | 4-cyano-2,6-difluorobenzyl |
| I-083 | cyclopropyl | H | H | 4-methoxybenzyl |
| I-084 | cyclopropyl | H | H | 4-nitrobenzyl |
| I-085 | cyclopropyl | H | H | 4-nitrobenzyl |
| I-086 | cyclopropyl | H | H | 4-cyano-2,5-difluorobenzyl |
| I-087 | isopropyl | H | H | 1-(4-cyanophenyl)ethyl |
| I-088 | 4-(trifluoromethyl)benzyl | H | H | cyclopropyl |
| I-089 | cyclopropyl | H | H | cyclopropyl |
| I-090 | cyclopropyl | H | H | (6-cyanopyridin-3-yl)methyl |
| I-091 | cyclopropyl | H | H | 2-(3-cyanophenyl)ethyl |
| I-092 | cyclopropyl | H | H | 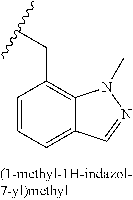 [SMILES: *Cc1cccc2cnn(C)c12] |
| I-093 | cyclopropyl | H | H | 4-(1,2,4-oxadiazol-3-yl)benzyl |
| I-094 | cyclopropyl | H | H | 4-(1,2,4-oxadiazol-3-yl)benzyl |
| I-095 | cyclopropyl | H | H | (2,2-difluoro-1,3-benzodioxol-5-yl)methyl |
| I-096 | cyclopropyl | H | H | 4-cyanobenzyl |
| I-097 | cyclopropyl | H | H | (6-cyanopyridin-3-yl)methyl |
| I-098 | cyclopropyl | H | H | 4-cyanobenzyl |
| I-099 | cyclopropyl | H | H | 4-cyanobenzyl |
| I-100 | cyclopropyl | H | H | 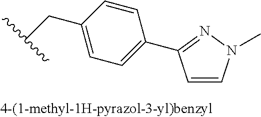 [SMILES: *Cc1ccc(-c2ccn(C)n2)cc1] |
| I-101 | cyclopropyl | H | H | 4-(methoxycarbonyl)benzyl |
| I-102 | cyclopropyl | H | H | 2-fluoro-4-nitrobenzyl |
| I-103 | cyclopropyl | H | H | cyclopropyl |
| I-104 | isopropyl | H | H | 4-(1,3-oxazol-5-yl)benzyl |
| I-105 | cyclopropyl | H | H | cyclopropyl |
| I-106 | cyclopropyl | H | H | (5-cyanopyridin-2-yl)methyl |
| I-107 | cyclopropyl | H | H | 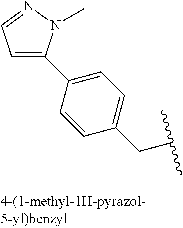 [SMILES: *Cc1ccc(-c2ccnn2C)cc1] |
| I-108 | cyclopropyl | H | H | 2-cyanopropan-2-yl |
| I-109 | cyclopropyl | H | H | 1-cyanocyclobutyl |
| I-110 | isopropyl | H | H | 4-cyano-2,5-difluorobenzyl |
| I-111 | cyclopropyl | H | H | 4-cyano-3,5-difluorobenzyl |
| I-112 | cyclopropyl | H | H | 4-(1,3,4-oxadiazol-2-yl)benzyl |
| I-113 | cyclopropyl | H | H | n-butyl |
| I-114 | cyclopropyl | H | H | 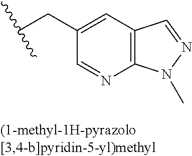 [SMILES: *Cc1cnc2c(cnn2C)c1] |
| I-115 | cyclopropyl | H | H | 1-cyanoethyl |
| I-116 | cyclopropyl | H | H | pyridin-2-ylmethyl |
| I-117 | cyclopropyl | H | H | oxetan-3-yl |
| I-118 | cyclopropyl | H | H | ethyl |
| I-119 | cyclopropyl | H | H | 4-cyanobenzyl |
| I-120 | cyclopropyl | H | H | 4-cyano-3-fluorobenzyl |
| I-121 | cyclopropyl | H | H | 4-cyanobenzyl |
| I-122 | cyclopropyl | H | H | 4-cyanobenzyl |
| I-123 | cyclopropyl | H | H | 4-cyanobenzyl |
| I-124 | cyclopropyl | H | H | 4-cyanobenzyl |
| I-125 | 4-bromobenzyl | H | H | cyclopropyl |
| I-126 | cyclopropyl | H | H | 1-ethynylcyclopropyl |
| I-127 | cyclopropyl | H | H | methyl |
| I-128 | cyclopropyl | H | H | 1-methylcyclopropyl |
| I-129 | cyclopropyl | H | H | isopropyl |
| I-130 | cyclopropyl | H | H | but-3-yn-2-yl |
| I-131 | cyclopropyl | H | H | cyanomethyl |
| I-132 | cyclopropyl | H | H | n-propyl |
| I-133 | cyclopropyl | H | H | cyclobutyl |
| I-134 | cyclopropyl | H | H | 4-cyanotetrahydro-2H-pyran-4-yl |
| I-135 | cyclopropyl | H | H | 2-oxotetrahydrofuran-3-yl |
| I-136 | cyclopropyl | H | H | 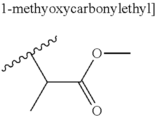 [SMILES: *C(C)C(=O)OC] |
| I-137 | cyclopropyl | H | H | 1-(trifluoromethyl)cyclopropyl |
| I-138 | cyclopropyl | H | H | 1-(methoxycarbonyl)cyclopropyl |
| I-139 | cyclopropyl | H | H | 3-cyanopentan-3-yl |
| I-140 | cyclopropyl | H | H | 1-methoxycyclopropyl |
| I-141 | cyclopropyl | H | H | cyclopropylmethyl |
| I-142 | cyclopropyl | H | H | 4-cyanobenzyl |
| I-143 | cyclopropyl | H | H | 4-cyanobenzyl |
| I-144 | cyclopropyl | H | H | 4-cyanobenzyl |
| I-145 | cyclopropyl | H | H | cis-4-(trifluoromethyl)cyclohexyl |
| I-146 | cyclopropyl | H | H | trans-4-(trifluoromethyl)cyclohexyl |
| I-147 | 1-methylcyclopropyl | H | H | 4-cyanobenzyl |
| I-148 | cyclopropyl | H | H | 4-cyanobenzyl |
| I-149 | cyclopropyl | H | H | 4-cyanobenzyl |
| I-150 | cyclopropyl | H | H | 4-cyanobenzyl |
| I-151 | cyclopropyl | H | H | 4-cyanobenzyl |
| I-152 | cyclopropyl | H | H | 4-cyanobenzyl |
| I-153 | cyclopropyl | H | H | 4-cyanobenzyl |
| I-154 | cyclopropyl | H | H | 4-cyanobenzyl |
| I-155 | cyclopropyl | H | H | 4-cyanobenzyl |
| I-156 | cyclopropyl | H | H | [cis-4-(trifluoromethyl)cyclohexyl]methyl |
| I-157 | cyclopropyl | H | H | 4-cyanobutyl |
| I-158 | cyclopropyl | H | H | 1-(4-cyanophenyl)ethyl |
| I-159 | cyclopropyl | H | H | 1-(4-cyanophenyl)cyclopropyl |
| I-160 | cyclopropyl | H | H | 4-cyanobenzyl |
| I-161 | cyclopropyl | H | H | (trans-4-cyanocyclohexyl)methyl |
| I-162 | cyclopropyl | H | H | thietan-3-yl |
| I-163 | cyclopropyl | H | H | 3-methylthietan-3-yl |
| I-164 | cyclopropyl | H | H | 5-cyano-2,3-dihydro-1H-inden-1-yl |
| I-165 | cyclopropyl | H | 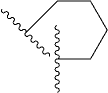 [SMILES: *CCCC*] | |
| I-166 | cyclopropyl | H | H | 5-cyano-1H-indazol-3-yl |
| I-167 | cyclopropyl | H | H | cyclohexylmethyl |
| I-168 | cyclopropyl | H | H | cyclohexyl |
| I-169 | cyclopropyl | H | H | cyclopentyl |
| I-170 | cyclopropyl | H | H | cyclopentylmethyl |
| I-171 | cyclopropyl | H | H | 4-cyanobenzyl |
| I-172 | cyclopropyl | H | H | 4-cyanobenzyl |
| I-173 | cyclopropyl | H | H | 4-cyanobenzyl |
| I-174 | cyclopropyl | H | H | 4-cyanobenzyl |
| I-175 | cyclopropyl | H | H | 3-(methoxycarbonyl)oxetan-3-yl |
| I-176 | cyclopropyl | H | H | 3,3,3-trifluoropropyl |
| I-177 | cyclopropyl | H | H | 2-methoxyethyl |
| I-178 | cyclopropyl | H | H | 4-cyanobenzyl |
| I-179 | cyclopropyl | H | H | 4-cyanobenzyl |
| I-180 | cyclopropyl | H | H | 4-cyanobenzyl |
| I-181 | cyclopropyl | H | H | 4-cyanobenzyl |
| I-182 | cyclopropyl | H | H | 4-cyanobenzyl |
| I-183 | cyclopropyl | H | Methoxy | methyl |
| I-184 | cyclopropyl | H | H | 1-cyclopropylcyclopropyl |
| I-185 | cyclopropyl | H | H | (trans-4-cyanocyclohexyl)methyl |
| I-186 | cyclopropyl | H | H | 4-aminosulphonylbenzyl |
| I-187 | cyclopropyl | H | H | 1,1-dioxidotetrahydrothiophen-3-yl |
| I-188 | cyclopropyl | H | H | 4-cyanobenzyl |
| I-189 | cyclopropyl | H | H | 1-thiocarbamoylcyclopropyl |
| I-190 | cyclopropyl | H | H | 4-cyanobenzyl |
| I-191 | cyclopropyl | H | H | 4-cyanobenzyl |
| I-192 | cyclopropyl | H | H | 4-cyanobenzyl |
| I-193 | cyclopropyl | H | H | 4-cyanobenzyl |
| I-194 | cyclopropyl | H | H | 4-cyanobenzyl |
| I-195 | cyclopropyl | H | H | (trans-4-cyanocyclohexyl)methyl |
| I-196 | cyclopropyl | H | H | 4-nitrobenzyl |
| I-197 | cyclopropyl | H | H | 4-cyanocyclohexyl |
| I-198 | cyclopropyl | H | H | (4-cyanocyclohexyl)methyl |
| I-199 | cyclopropyl | H | 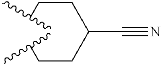 [SMILES: *CCC(C#N)CC*] | |
| I-200 | cyclopropyl | H | H | 5-cyanopentyl |
| I-201 | cyclopropyl | H | 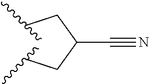 [SMILES: *CC(C#N)C*] | |
| I-202 | cyclopropyl | H | 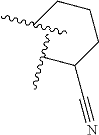 [SMILES: *CCCC(*)C#N] | |
| I-203 | 1-cyclopropylcyclopropyl | H | H | 4-cyanobenzyl |
| I-204 | methyl | H | H | 4-cyanobenzyl |
| I-205 | 1-chlorocyclopropyl | H | H | 4-cyanobenzyl |
| I-206 | tert-butyl | H | H | 4-cyanobenzyl |
| I-207 | cylopentyl | H | H | 4-cyanobenzyl |
| I-208 | cyclopropyl | H | H | 4-cyanobenzyl |
| I-209 | cyclopropyl | H | H | 4-nitrobenzyl |
| I-210 | 1-cyanocyclopropyl | H | H | 4-cyanobenzyl |
| I-211 | 4-nitrobenzyl | H | H | 4-cyanobenzyl |
| I-212 | 1,3-oxazol-5-yl | H | H | 4-cyanobenzyl |
| I-213 | 3,3-difluorocyclobutyl | H | H | 4-cyanobenzyl |
| I-214 | cyclopropyl | H | H | 4-thiocarbamoylbenzyl |
| I-215 | cyclopropyl | Methyl | H | 4-cyanobenzyl |
| I-216 | cyclopropyl | H | H | 2,2-difluorocyclopropyl |
| I-217 | isopropyl | Methyl | H | 4-cyanobenzyl |
| I-218 | cyclopropyl | H | H | 1-(4-cyanophenyl)ethyl |
| I-219 | cyclopropyl | H | H | 1-(4-cyanophenyl)ethyl |
| I-220 | methyl | methyl | H | 4-cyanobenzyl |
| I-221 | isopropyl | ethyl | H | 4-cyanobenzyl |
| I-222 | cyclopropyl | H | H | 4-cyanobenzyl |
| I-223 | 1-methoxyethyl | H | H | 4-cyanobenzyl |
| I-224 | cyclopropyl | H | H | 4-cyanobenzyl |
| I-225 | cyclopropyl | H | H | 4-cyanobenzyl |
| I-226 | cyclopropyl | H | H | 4-fluoro-3-(trifluoromethyl)benzyl |
| I-227 | cyclopropyl | H | H | benzyl |
| I-228 | cyclopropyl | H | H | 1-(4-nitrophenyl)ethyl |
| I-229 | cyclopropyl | H | H | 1-(4-nitrophenyl)cyclopropyl |
| I-230 | cyclopropyl | H | H | 4-cyanobenzyl |
| I-231 | cyclopropyl | H | methoxycarbonyl | 4-cyanobenzyl |
| I-232 | cyclopropyl | methoxycarbonyl | methoxycarbonyl | 4-cyanobenzyl |
| I-233 | cyclopropyl | methoxycarbonyl | H | 4-cyanobenzyl |
| I-234 | cyclopropyl | H | H | methoxy |
| I-235 | cyclopropyl | H | H | ethoxy |
| I-236 | cyclopropyl | H | H | cyclopropylmethyloxy |
| I-237 | cyclopropyl | H | H | isopropyloxy |
| I-238 | cyclopropyl | H | H | 3-cyanopropyloxy |
| I-239 | cyclopropyl | H | H | 4-cyanophenyloxy |
| I-240 | cyclopropyl | H | H | (5-nitro-2-thienyl)methyl |
| I-241 | 2,2,2-trifluoroethyl | H | H | 4-cyanobenzyl |
| I-242 | 1-(trifluoromethyl)cyclopropyl | H | H | 4-cyanobenzyl |
| I-243 | cyclopropyl | H | H | cyclopropyl |
| I-244 | cyclopropyl | H | H | 1-methoxycyclopropyl |
| I-245 | cyclopropyl | H | H | 1-cyanoethyl |
| I-246 | cyclopropyl | H | H | 3,3,3-trifluoropropyl |
| I-247 | cyclopropyl | H | H | 1-(4-cyanophenyl)ethyl |
| I-248 | cyclopropyl | H | H | 4-cyano-2,6-difluorobenzyl |
| I-249 | cyclopropyl | H | H | 4-cyano-2,5-difluorobenzyl |
| I-250 | cyclopropyl | H | H | 4-cyano-3,5-difluorobenzyl |
| I-251 | cyclopropyl | H | H | 4-cyano-2-fluorobenzyl |
| I-252 | cyclopropyl | H | H | 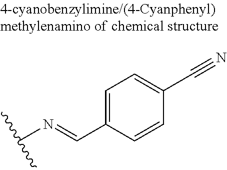 [SMILES: */N=C/c1ccc(C#N)cc1] |
| I-253 | cyclopropyl | H | H | 2-methyl-1-methylsulphonylpropan-2-yl |
| I-254 | cyclopropyl | H | H | 4-cyanobenzyl |
| I-255 | cyclopropyl | H | H | (5-cyano-2-thienyl)methyl |
| I-256 | 6-chloropyridin-3-yl | H | H | 4-cyanobenzyl |
| I-257 | cyclopropyl | H | H | 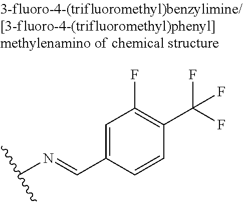 [SMILES: */N=C/c1ccc(C(F)(F)F)c(F)c1] |
| I-258 | cyclopropyl | H | H | 4-cyanobenzyl |
| I-259 | cyclopropyl | H | H | [4-(2-oxopyrrolidin-1-yl)phenyl]methyl |
| I-260 | 4-(trifluoromethyl)benzyl | H | H | 4-cyanobenzyl |
| I-261 | cyclopropyl | H | H | 4-cyanophenyl |
| I-262 | cyclopropyl | H | H | 4-cyanobenzyl |
| I-263 | cyclopropyl | H | H | 4-nitrobenzoyl |
| I-264 | cyclopropyl | H | H | cyclopropylcarbonyl |
| I-265 | cyclopropyl | H | H | 4-cyanobenzyl |
| I-266 | cyclopropyl | H | H | 4-cyanobenzyl |
| I-267 | cyclopropyl | H | H | 4-cyanobenzyl |
| I-268 | cyclopropyl | H | H | 4-cyanobenzyl |
| I-269 | cyclopropyl | H | H | 4-cyanobenzyl |
| I-270 | cyclopropyl | H | H | 4-cyanobenzyl |
| I-271 | cyclopropyl | H | H | 4-cyanobenzyl |
| I-272 | cyclopropyl | H | H | 4-cyanobenzyl |
| I-273 | cyclopropyl | H | H | 4-cyanobenzyl |
| I-274 | cyclopropyl | H | H | 4-cyanobenzyl |
| I-275 | cyclopropyl | H | H | 4-cyanobenzyl |
| I-276 | cyclopropyl | H | H | 4-cyanobenzyl |
| I-277 | cyclopropyl | H | H | (pyridin-4-yl)methyl |
| I-278 | cyclopropyl | H | H | 4-cyanobenzyl |

| Ex. | R5 | R6 | V1 | V2 | Q1 | Further descriptor |
|---|---|---|---|---|---|---|
| I-001 | H | H | O | O | 4-fluorophenyl | |
| I-002 | H | H | O | O | 4-fluorophenyl | |
| I-003 | H | H | O | O | 4-(trifluoromethyl)phenyl | |
| I-004 | H | H | O | O | 4-(trifluoromethyl)phenyl | |
| I-005 | H | H | O | O | 4-(trifluoromethyl)phenyl | |
| I-006 | H | H | O | O | 4-(trifluoromethyl)phenyl | |
| I-007 | H | H | O | O | 4-(trifluoromethyl)phenyl | |
| I-008 | H | H | O | O | 4-(trifluoromethyl)phenyl | |
| I-009 | H | H | O | O | 4-(trifluoromethyl)phenyl | |
| I-010 | H | H | O | O | 4-(trifluoromethyl)phenyl | |
| I-011 | H | H | O | O | 4-(trifluoromethyl)phenyl | |
| I-012 | H | H | O | O | 4-(trifluoromethyl)phenyl | |
| I-013 | H | H | O | O | 4-(trifluoromethyl)phenyl | |
| I-014 | H | H | O | O | 4-(trifluoromethyl)phenyl | |
| I-015 | H | H | O | O | 4-(trifluoromethyl)phenyl | |
| I-016 | H | H | O | O | 4-(trifluoromethyl)phenyl | |
| I-017 | H | H | O | O | 4-(trifluoromethyl)phenyl | |
| I-018 | H | H | O | O | 4-(trifluoromethyl)phenyl | |
| I-019 | H | H | O | O | 4-(trifluoromethyl)phenyl | |
| I-020 | H | H | O | O | 4-chlorophenyl | |
| I-021 | H | H | O | O | 4-(trifluoromethyl)phenyl | |
| I-022 | H | H | O | O | 4-chlorophenyl | |
| I-023 | H | H | O | O | 4-(trifluoromethyl)phenyl | |
| I-024 | H | H | O | O | 4-chlorophenyl | |
| I-025 | H | H | O | O | 4-(trifluoromethyl)phenyl | |
| I-026 | H | H | O | O | 4-(trifluoromethyl)phenyl | |
| I-027 | H | H | O | O | 4-(trifluoromethyl)phenyl | |
| I-028 | H | H | O | O | 4-(trifluoromethyl)phenyl | |
| I-029 | H | H | O | O | 4-(difluoromethoxy)phenyl | |
| I-030 | H | H | O | O | 4-(difluoromethoxy)phenyl | |
| I-031 | H | H | O | O | 4-(trifluoromethyl)phenyl | |
| I-032 | H | H | O | O | 4-(trifluoromethyl)phenyl | |
| I-033 | H | H | O | O | 4-(trifluoromethyl)phenyl | |
| I-034 | H | H | O | O | 4-(trifluoromethyl)phenyl | |
| I-035 | H | H | O | O | 4-iodophenyl | |
| I-036 | H | H | O | O | 4-(trifluoromethyl)phenyl | |
| I-037 | H | H | O | O | 4-(trifluoromethyl)phenyl | |
| I-038 | H | H | O | O | 4-(trifluoromethyl)phenyl | |
| I-039 | H | H | O | O | 4-(trifluoromethyl)phenyl | |
| I-040 | H | H | O | O | 4-(trifluoromethyl)phenyl | |
| I-041 | H | H | O | O | 4-(trifluoromethyl)phenyl | |
| I-042 | H | H | O | O | 4-(trifluoromethyl)phenyl | |
| I-043 | H | H | O | O | 4-(trifluoromethyl)phenyl | |
| I-044 | H | H | O | O | 4-(trifluoromethoxy)phenyl | |
| I-045 | H | H | O | O | 4-(trifluoromethyl)phenyl | |
| I-046 | H | H | O | O | 4-(trifluoromethyl)phenyl | |
| I-047 | H | H | O | O | 4-(trifluoromethyl)phenyl | |
| I-048 | H | H | O | O | 4-(trifluoromethyl)phenyl | |
| I-049 | H | H | O | O | 4-(trifluoromethyl)phenyl | |
| I-050 | H | H | O | O | 4-iodophenyl | |
| I-051 | H | H | O | O | 4-(trifluoromethoxy)phenyl | |
| I-052 | H | H | O | O | 4-[(trifluoromethyl)sulphanyl]phenyl | |
| I-053 | H | H | O | O | 4-bromophenyl | |
| I-054 | H | H | O | O | 4-(trifluoromethyl)phenyl | |
| I-055 | H | H | O | O | 4-(trifluoromethyl)phenyl | |
| I-056 | H | H | O | O | 4-(trifluoromethyl)phenyl | |
| I-057 | H | H | O | O | 4-(trifluoromethyl)phenyl | |
| I-058 | H | H | O | O | 4-(trifluoromethyl)phenyl | |
| I-059 | H | H | O | O | 4-(trifluoromethyl)phenyl | |
| I-060 | H | H | O | O | 4-(trifluoromethyl)phenyl | |
| I-061 | H | H | O | O | 4-(trifluoromethoxy)phenyl | |
| I-062 | H | H | O | O | 4-(trifluoromethoxy)phenyl | |
| I-063 | H | H | O | O | 4-(trifluoromethoxy)phenyl | |
| I-064 | H | H | O | O | 4-(trifluoromethyl)phenyl | |
| I-065 | H | H | O | O | 4-(trifluoromethoxy)phenyl | |
| I-066 | H | H | O | O | 4-(trifluoromethyl)phenyl | |
| I-067 | H | H | O | O | 4-(trifluoromethoxy)phenyl | |
| I-068 | H | H | O | O | 4-(trifluoromethyl)phenyl | |
| I-069 | H | H | O | O | 4-(trifluoromethyl)phenyl | |
| I-070 | H | H | O | O | 4-(trifluoromethoxy)phenyl | |
| I-071 | H | H | O | O | 4-(trifluoromethyl)phenyl | |
| I-072 | H | H | O | O | 4-(trifluoromethyl)phenyl | |
| I-073 | H | H | O | O | 4-(trifluoromethoxy)phenyl | |
| I-074 | H | H | O | O | 4-(trifluoromethyl)phenyl | |
| I-075 | H | H | O | O | 4-(trifluoromethoxy)phenyl | |
| I-076 | H | H | O | O | 4-(trifluoromethoxy)phenyl | |
| I-077 | H | H | O | O | 4-(trifluoromethyl)phenyl | |
| I-078 | H | H | O | O | 4-(trifluoromethyl)phenyl | |
| I-079 | H | H | O | O | 4-(trifluoromethoxy)phenyl | |
| I-080 | H | H | O | O | 4-(trifluoromethoxy)phenyl | |
| I-081 | H | H | O | O | 4-(trifluoromethoxy)phenyl | |
| I-082 | H | H | O | O | 4-(trifluoromethoxy)phenyl | |
| I-083 | H | H | O | O | 4-(trifluoromethoxy)phenyl | |
| I-084 | H | H | O | O | 4-(trifluoromethyl)phenyl | |
| I-085 | H | H | O | O | 4-(trifluoromethoxy)phenyl | |
| I-086 | H | H | O | O | 4-(trifluoromethoxy)phenyl | |
| I-087 | H | H | O | O | 4-(trifluoromethyl)phenyl | |
| I-088 | H | H | O | O | 4-(trifluoromethyl)phenyl | |
| I-089 | H | H | O | O | 4-(trifluoromethoxy)phenyl | |
| I-090 | H | H | O | O | 4-[(trifluoromethyl)sulphanyl]phenyl | |
| I-091 | H | H | O | O | 4-(trifluoromethoxy)phenyl | |
| I-092 | H | H | O | O | 4-(trifluoromethoxy)phenyl | |
| I-093 | H | H | O | O | 4-[(trifluoromethyl)sulphanyl]phenyl | |
| I-094 | H | H | O | O | 4-(trifluoromethoxy)phenyl | |
| I-095 | H | H | O | O | 4-(trifluoromethoxy)phenyl | |
| I-096 | H | H | O | O | 4-[(trifluoromethyl)sulphonyl]phenyl | |
| I-097 | H | H | O | O | 4-[(trifluoromethyl)sulphonyl]phenyl | |
| I-098 | H | H | O | O | 3-methyl-4-(trifluoromethoxy)phenyl | |
| I-099 | H | H | O | O | 2-methyl-4-(trifluoromethoxy)phenyl | |
| I-100 | H | H | O | O | 4-(trifluoromethoxy)phenyl | |
| I-101 | H | H | O | O | 4-(trifluoromethoxy)phenyl | |
| I-102 | H | H | O | O | 4-(trifluoromethoxy)phenyl | |
| I-103 | H | H | O | O | 4-[(trifluoromethyl)sulphonyl]phenyl | |
| I-104 | H | H | O | O | 4-(trifluoromethoxy)phenyl | |
| I-105 | H | H | O | O | 4-[(trifluoromethyl)sulphanyl]phenyl | |
| I-106 | H | H | O | O | 4-(trifluoromethoxy)phenyl | |
| I-107 | H | H | O | O | 4-(trifluoromethoxy)phenyl | |
| I-108 | H | H | O | O | 4-(trifluoromethoxy)phenyl | |
| I-109 | H | H | O | O | 4-(trifluoromethoxy)phenyl | |
| I-110 | H | H | O | O | 4-(trifluoromethoxy)phenyl | |
| I-111 | H | H | O | O | 4-(trifluoromethoxy)phenyl | |
| I-112 | H | H | O | O | 4-(trifluoromethoxy)phenyl | |
| I-113 | H | H | O | O | 4-(trifluoromethoxy)phenyl | |
| I-114 | H | H | O | O | 4-(trifluoromethoxy)phenyl | |
| I-115 | H | H | O | O | 4-(trifluoromethoxy)phenyl | |
| I-116 | H | H | O | O | 4-(trifluoromethoxy)phenyl | |
| I-117 | H | H | O | O | 4-(trifluoromethoxy)phenyl | |
| I-118 | H | H | O | O | 4-(trifluoromethoxy)phenyl | |
| I-119 | H | H | O | O | 3-chloro-5-(trifluoromethyl)pyridin-2-yl | |
| I-120 | H | H | O | O | 4-[(trifluoromethyl)sulphonyl]phenyl | |
| I-121 | H | H | O | O | 3-fluoro-4-(trifluoromethoxy)phenyl | |
| I-122 | H | H | O | O | 2-chloro-4-(trifluoromethoxy)phenyl | |
| I-123 | H | H | O | O | 6-(trifluoromethyl)pyridin-3-yl | |
| I-124 | H | H | O | O | 2-fluoro-4-(trifluoromethoxy)phenyl | |
| I-125 | H | H | O | O | 4-(trifluoromethoxy)phenyl | |
| I-126 | H | H | O | O | 4-(trifluoromethoxy)phenyl | |
| I-127 | H | H | O | O | 4-(trifluoromethoxy)phenyl | |
| I-128 | H | H | O | O | 4-(trifluoromethoxy)phenyl | |
| I-129 | H | H | O | O | 4-(trifluoromethoxy)phenyl | |
| I-130 | H | H | O | O | 4-(trifluoromethoxy)phenyl | |
| I-131 | H | H | O | O | 4-(trifluoromethoxy)phenyl | |
| I-132 | H | H | O | O | 4-(trifluoromethoxy)phenyl | |
| I-133 | H | H | O | O | 4-(trifluoromethoxy)phenyl | |
| I-134 | H | H | O | O | 4-(trifluoromethoxy)phenyl | |
| I-135 | H | H | O | O | 4-(trifluoromethoxy)phenyl | |
| I-136 | H | H | O | O | 4-(trifluoromethoxy)phenyl | |
| I-137 | H | H | O | O | 4-(trifluoromethoxy)phenyl | |
| I-138 | H | H | O | O | 4-(trifluoromethoxy)phenyl | |
| I-139 | H | H | O | O | 4-(trifluoromethoxy)phenyl | |
| I-140 | H | H | O | O | 4-(trifluoromethoxy)phenyl | |
| I-141 | H | H | O | O | 4-(trifluoromethoxy)phenyl | |
| I-142 | H | H | O | O | 4-(cyclopropylsulphanyl)phenyl | |
| I-143 | H | H | O | O | 2-fluoro-6-(trifluoromethyl)pyridin-3-yl | |
| I-144 | H | H | O | O | 4-(2,2,2-trifluoroethyl)phenyl | |
| I-145 | H | H | O | O | 4-(trifluoromethoxy)phenyl | |
| I-146 | H | H | O | O | 4-(trifluoromethoxy)phenyl | |
| I-147 | H | H | O | O | 4-(trifluoromethoxy)phenyl | |
| I-148 | H | H | O | O | 2-ethyl-6-methyl-4-(pentafluoroethyl)phenyl | |
| I-149 | H | H | O | O | 3-fluoro-4-(pentafluoroethyl)phenyl | |
| I-150 | H | H | O | O | 4-(1,1,1,2,3,3,3-heptafluoropropan-2-yl)phenyl | |
| I-151 | H | H | O | O | 4-[(trifluoromethyl)sulphinyl]phenyl | |
| I-152 | H | H | O | O | 3,5-difluoro-4-(trifluoromethoxy)phenyl | |
| I-153 | H | H | O | O | 4-(1,1,2,2-tetrafluoroethoxy)phenyl | |
| I-154 | H | H | O | O | 2,2-difluoro-1,3-benzodioxol-5-yl | |
| I-155 | H | H | O | O | 4-(difluoromethoxy)-3,5-difluorophenyl | |
| I-156 | H | H | O | O | 4-(trifluoromethoxy)phenyl | |
| I-157 | H | H | O | O | 4-(trifluoromethoxy)phenyl | |
| I-158 | H | H | O | O | 4-(trifluoromethoxy)phenyl | Racemate |
| I-159 | H | H | O | O | 4-(trifluoromethoxy)phenyl | |
| I-160 | H | H | O | O | 5-(trifluoromethyl)-2-thienyl | |
| I-161 | H | H | O | O | 4-(trifluoromethoxy)phenyl | |
| I-162 | H | H | O | O | 4-(trifluoromethoxy)phenyl | |
| I-163 | H | H | O | O | 4-(trifluoromethoxy)phenyl | |
| I-164 | H | H | O | O | 4-(trifluoromethoxy)phenyl | |
| I-165 | H | H | O | O | 4-(trifluoromethoxy)phenyl | |
| I-166 | H | H | O | O | 4-(trifluoromethoxy)phenyl | |
| I-167 | H | H | O | O | 4-(trifluoromethoxy)phenyl | |
| I-168 | H | H | O | O | 4-(trifluoromethoxy)phenyl | |
| I-169 | H | H | O | O | 4-(trifluoromethoxy)phenyl | |
| I-170 | H | H | O | O | 4-(trifluoromethoxy)phenyl | |
| I-171 | H | H | O | O | 4-[(trifluorovinyl)oxy]phenyl | |
| I-172 | H | H | O | O | 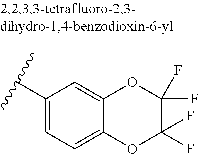 [SMILES: *c1ccc2c(c1)OC(F)(F)C(F)(F)O2] | |
| I-173 | H | H | O | O | 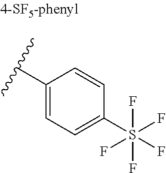 [SMILES: *c1ccc(S(F)(F)(F)(F)F)cc1] | |
| I-174 | H | H | O | O | 5-(trifluoromethyl)pyridin-2-yl | |
| I-175 | H | H | O | O | 4-(trifluoromethoxy)phenyl | |
| I-176 | H | H | O | O | 4-(trifluoromethoxy)phenyl | |
| I-177 | H | H | O | O | 4-(trifluoromethoxy)phenyl | |
| I-178 | H | H | O | O | 4-[(1,1,2,2-tetrafluoroethyl)sulphanyl]phenyl | |
| I-179 | H | H | O | O | 4-[(difluoromethyl)sulphinyl]phenyl | |
| I-180 | H | H | O | O | 4-[(difluoromethyl)sulphanyl]phenyl | |
| I-181 | H | H | S | S | 4-(trifluoromethoxy)phenyl | |
| I-182 | H | H | O | O | 4-(pentafluoroethyl)phenyl | |
| I-183 | H | H | O | O | 4-(trifluoromethoxy)phenyl | |
| I-184 | H | H | O | O | 4-(trifluoromethoxy)phenyl | |
| I-185 | H | H | O | O | 3-fluoro-4-(pentafluoroethyl)phenyl | |
| I-186 | H | H | O | O | 4-(trifluoromethoxy)phenyl | |
| I-187 | H | H | O | O | 4-(trifluoromethoxy)phenyl | |
| I-188 | H | H | O | O | 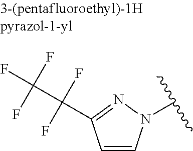 [SMILES: *n1ccc(C(F)(F)C(F)(F)F)n1] | |
| I-189 | H | H | O | O | 4-(trifluoromethoxy)phenyl | |
| I-190 | H | H | O | O | 4-[chloro(difluoro)methoxy]phenyl | |
| I-191 | H | H | O | O | 4-(pentafluoroethoxy)phenyl | |
| I-192 | H | H | O | O | 4-(1,1-difluoroethyl)phenyl | |
| I-193 | H | H | O | O | 4-[1-(trifluoromethyl)cyclopropyl]phenyl | |
| I-194 | H | H | O | O | 4-(2-chloro-1,1,2-trifluoroethoxy)phenyl | |
| I-195 | H | H | O | O | 4-(pentafluoroethyl)phenyl | |
| I-196 | H | H | O | O | 4-(pentafluoroethyl)phenyl | |
| I-197 | H | H | O | O | 4-(trifluoromethoxy)phenyl | |
| I-198 | H | H | O | O | 4-(trifluoromethoxy)phenyl | |
| I-199 | H | H | O | O | 4-(trifluoromethoxy)phenyl | |
| I-200 | H | H | O | O | 4-(trifluoromethoxy)phenyl | |
| I-201 | H | H | O | O | 4-(trifluoromethoxy)phenyl | |
| I-202 | H | H | O | O | 4-(trifluoromethoxy)phenyl | |
| I-203 | H | H | O | O | 4-(trifluoromethoxy)phenyl | |
| I-204 | H | H | O | O | 4-(trifluoromethoxy)phenyl | |
| I-205 | H | H | O | O | 4-(trifluoromethoxy)phenyl | |
| I-206 | H | H | O | O | 4-(trifluoromethoxy)phenyl | |
| I-207 | H | H | O | O | 4-(trifluoromethoxy)phenyl | |
| I-208 | H | H | O | O | 3-(trifluoromethoxy)phenyl | |
| I-209 | H | H | O | O | 3-fluoro-4-(pentafluoroethyl)phenyl | |
| I-210 | H | H | O | O | 4-(trifluoromethoxy)phenyl | |
| I-211 | H | H | O | O | 4-(trifluoromethoxy)phenyl | |
| I-212 | H | H | O | O | 4-(trifluoromethoxy)phenyl | |
| I-213 | H | H | O | O | 4-(trifluoromethoxy)phenyl | |
| I-214 | H | H | O | O | 4-(trifluoromethoxy)phenyl | |
| I-215 | H | H | O | O | 4-(trifluoromethoxy)phenyl | |
| I-216 | H | H | O | O | 4-(trifluoromethoxy)phenyl | |
| I-217 | H | H | O | O | 4-(trifluoromethoxy)phenyl | |
| I-218 | H | H | O | O | 4-(trifluoromethoxy)phenyl | (□) enantiomer |
| I-219 | H | H | O | O | 4-(trifluoromethoxy)phenyl | (□) enantiomer |
| I-220 | H | H | O | O | 4-(trifluoromethoxy)phenyl | |
| I-221 | H | H | O | O | 4-(trifluoromethoxy)phenyl | |
| I-222 | H | H | O | O | 4-(difluoromethyl)-3-fluorphenyl | |
| I-223 | H | H | O | O | 4-(trifluoromethoxy)phenyl | |
| I-224 | H | H | O | O | 3-fluoro-4-(trifluoromethyl)phenyl | |
| I-225 | H | H | O | O | 4-[bis(trifluoromethyl)amino]phenyl | |
| I-226 | H | H | O | O | 4-(trifluoromethoxy)phenyl | |
| I-227 | H | H | O | O | 4-(trifluoromethoxy)phenyl | |
| I-228 | H | H | O | O | 4-(trifluoromethoxy)phenyl | |
| I-229 | H | H | O | O | 4-(trifluoromethoxy)phenyl | |
| I-230 | H | H | O | O | 3-chloro-4-(trifluoromethoxy)phenyl | |
| I-231 | H | H | O | O | 4-(trifluoromethoxy)phenyl | |
| I-232 | H | H | O | O | 4-(trifluoromethoxy)phenyl | |
| I-233 | H | H | O | O | 4-(trifluoromethoxy)phenyl | |
| I-234 | H | H | O | O | 4-(trifluoromethoxy)phenyl | |
| I-235 | H | H | O | O | 4-(trifluoromethoxy)phenyl | |
| I-236 | H | H | O | O | 4-(trifluoromethoxy)phenyl | |
| I-237 | H | H | O | O | 4-(trifluoromethoxy)phenyl | |
| I-238 | H | H | O | O | 4-(trifluoromethoxy)phenyl | |
| I-239 | H | H | O | O | 4-(trifluoromethoxy)phenyl | |
| I-240 | H | H | O | O | 4-(trifluoromethoxy)phenyl | |
| I-241 | H | H | O | O | 4-(trifluoromethoxy)phenyl | |
| I-242 | H | H | O | O | 4-(trifluoromethoxy)phenyl | |
| I-243 | H | H | O | O | 3-fluoro-4-(pentafluoroethyl)phenyl | |
| I-244 | H | H | O | O | 3-fluoro-4-(pentafluoroethyl)phenyl | |
| I-245 | H | H | O | O | 3-fluoro-4-(pentafluoroethyl)phenyl | |
| I-246 | H | H | O | O | 3-fluoro-4-(pentafluoroethyl)phenyl | |
| I-247 | H | H | O | O | 3-fluoro-4-(pentafluoroethyl)phenyl | |
| I-248 | H | H | O | O | 3-fluoro-4-(pentafluoroethyl)phenyl | |
| I-249 | H | H | O | O | 3-fluoro-4-(pentafluoroethyl)phenyl | |
| I-250 | H | H | O | O | 3-fluoro-4-(pentafluoroethyl)phenyl | |
| I-251 | H | H | O | O | 3-fluoro-4-(pentafluoroethyl)phenyl | |
| I-252 | H | H | O | O | 4-(trifluoromethoxy)phenyl | |
| I-253 | H | H | O | O | 3-fluoro-4-(pentafluoroethyl)phenyl | |
| I-254 | H | H | O | O | 4-(1,1,2,3,3,3-hexafluoropropoxy)phenyl | |
| I-255 | H | H | O | O | 4-(trifluoromethoxy)phenyl | |
| I-256 | H | H | O | O | 4-(trifluoromethoxy)phenyl | |
| I-257 | H | H | O | O | 4-(trifluoromethoxy)phenyl | |
| I-258 | H | H | O | NH | 4-(trifluoromethoxy)phenyl | |
| I-259 | H | H | O | O | 4-(trifluoromethoxy)phenyl | |
| I-260 | H | H | O | O | 4-(trifluoromethoxy)phenyl | |
| I-261 | H | H | O | NH | 4-(trifluoromethoxy)phenyl | |
| I-262 | H | H | O | O | 4-bromo-3-fluorophenyl | |
| I-263 | H | H | O | O | 3-fluoro-4-(pentafluoroethyl)phenyl | |
| I-264 | H | H | O | O | 3-fluoro-4-(pentafluoroethyl)phenyl | |
| I-265 | H | H | O | O | 2,6-dichloro-4-(pentafluoroethyl)phenyl | |
| I-266 | H | H | O | O | 2-chloro-3-fluoro-4-(pentafluoroethyl)phenyl | |
| I-267 | H | H | O | O | 3-fluoro-5-methyl-4-(pentafluoroethyl)phenyl | |
| I-268 | H | H | O | O | 2,6-dichloro-4-(pentafluoroethyl)phenyl | |
| I-269 | H | H | O | O | 2-chloro-5-fluoro-4-(pentafluoroethyl)phenyl | |
| I-270 | H | H | O | O | 3-methyl-4-(pentafluoroethyl)phenyl | |
| I-271 | H | H | O | O | 3,5-difluoro-4-(pentafluoroethyl)phenyl | |
| I-272 | H | H | O | O | 2,3-difluoro-4-(pentafluoroethyl)phenyl | |
| I-273 | H | H | O | O | 3-chloro-4-(pentafluoroethyl)phenyl | |
| I-274 | H | H | O | O | 2,3,5-trifluoro-4-(pentafluoroethyl)phenyl | |
| I-275 | H | H | O | O | 2,5-difluoro-4-(pentafluoroethyl)phenyl | |
| I-276 | H | H | O | O | 2,6-dichloro-3-fluoro-4-(pentafluoroethyl)phenyl | |
| I-277 | H | H | O | O | 3-fluoro-4-(pentafluoroethyl)phenyl | |
| I-278 | H | H | O | O | 3-chloro-5-fluoro-4-(pentafluoroethyl)phenyl | |

| Example | LOGP_NEUTRAL | LOGP_HCOOH | NMR |
|---|---|---|---|
| I-001 | | 3.26 | Example I-001: ¹H-NMR(400.0 MHz, d₆-DMSO): δ = |
| | | | 10.929(2.7); 8.930(0.7); 8.914(1.5); 8.898(0.8); 8.501(3.2); 8.497(3.4); 7.958(3.5); |
| | | | 7.954(3.6); 7.845(1.9); 7.832(2.3); 7.823(3.8); 7.818(3.8); 7.767(1.5); 7.748(1.9); |
| | | | 7.720(1.4); 7.700(1.8); 7.598(1.7); 7.578(2.6); 7.559(1.1); 7.400(2.1); |
| | | | 7.378(3.9); 7.356(2.0); 4.620(3.2); 4.605(3.2); 3.903(16.0); 3.337(88.9); 2.673 |
| | | | (0.4); 2.508(48.6); 2.504(65.8); 2.500(52.7); 2.331(0.4); 2.048(0.7); 2.034(1.1); |
| | | | 2.021(0.8); 1.336(0.4); 1.259(0.5); 1.249(0.6); 1.233(1.7); 0.866(6.1); |
| | | | 0.857(4.0); 0.847(3.2); 0.000(0.6) |
| I-002 | | 3.24 | Example I-002: ¹H-NMR(400.0 MHz, d₆-DMSO): δ = |
| | | | 10.941(2.1); 8.955(0.6); 8.939(1.2); 8.923(0.6); 8.504(2.6); 8.500(2.7); 7.957(2.8); |
| | | | 7.953(2.8); 7.845(2.0); 7.839(3.9); 7.832(2.2); 7.823(3.1); 7.819(4.3); 7.810(1.8); |
| | | | 7.556(3.2); 7.535(2.8); 7.401(1.7); 7.379(3.2); 7.357(1.6); 4.655(2.4); |
| | | | 4.639(2.4); 3.903(16.0); 3.344(72.5); 2.509(35.6); 2.505(48.8); 2.500(37.2); |
| | | | 2.049(0.5); 2.035(0.8); 2.021(0.5); 1.259(0.3); 1.250(0.5); 1.233(1.2); |
| | | | 0.868(4.6); 0.858(2.9); 0.848(2.3); 0.000(0.5) |
| I-003 | | 4.24 | Example I-003: ¹H-NMR(400.0 MHz, d₆-DMSO): δ = |
| | | | 11.014(2.1); 8.868(0.6); 8.853(1.2); 8.837(0.6); 8.581(2.8); 8.577(2.8); 8.030(3.1); |
| | | | 8.027(3.1); 8.009(2.0); 7.989(3.1); 7.920(3.3); 7.899(2.1); 7.435(0.5); 7.419(0.8); |
| | | | 7.415(1.1); 7.399(1.2); 7.380(0.7); 7.222(1.5); 7.202(2.2); 7.178(0.9); |
| | | | 7.134(0.6); 7.128(0.5); 7.112(1.0); 7.107(0.8); 7.091(0.5); 7.086(0.4); 4.596(2.6); |
| | | | 4.580(2.6); 3.903(16.0); 3.343(115.0); 2.674(0.3); 2.513(21.3); 2.509(44.4); |
| | | | 2.504(62.2); 2.500(47.7); 2.331(0.4); 2.048(0.5); 2.041(0.4); 2.034(0.9); |
| | | | 2.020(0.6); 1.336(0.4); 1.249(0.5); 1.233(0.6); 1.225(0.4); 0.875(4.5); 0.866(2.9); |
| | | | 0.855(2.4); 0.833(0.4); 0.000(0.6) |
| I-004 | | 3.83 | Example I-004: ¹H-NMR(400.0 MHz, d₆-DMSO): δ = |
| | | | 11.003(3.0); 8.964(0.8); 8.948(1.7); 8.932(0.8); 8.577(3.6); 8.574(3.8); 8.026(3.9); |
| | | | 8.023(4.0); 8.008(2.7); 7.987(4.3); 7.919(4.4); 7.898(2.9); 7.823(3.1); 7.769(1.7); |
| | | | 7.750(2.0); 7.725(1.4); 7.705(1.9); 7.600(1.8); 7.581(2.8); 7.561(1.2); |
| | | | 4.629(3.3); 4.613(3.3); 3.903(16.0); 3.343(199.1); 2.674(0.4); 2.509(52.6); |
| | | | 2.504(71.6); 2.500(56.3); 2.331(0.4); 2.059(0.7); 2.045(1.1); 2.029(0.8); |
| | | | 1.249(0.5); 1.233(0.5); 0.877(6.5); 0.867(4.0); 0.857(3.2); 0.834(0.3); 0.000(0.5) |
| I-005 | | 3.73 | Example I-005: ¹H-NMR(400.0 MHz, d₆-DMSO): δ = |
| | | | 11.009(5.2); 10.034(0.3); 8.973(1.5); 8.958(3.1); 8.942(1.4); 8.580(6.8); 8.576 |
| | | | (6.7); 8.551(1.5); 8.316(0.6); 8.172(0.4); 8.151(0.5); 8.094(0.4); 8.024(7.7); |
| | | | 8.021(7.5); 8.008(4.9); 7.987(7.6); 7.937(0.7); 7.919(8.4); 7.898(5.1); 7.841(9.2); |
| | | | 7.820(10.8); 7.560(9.1); 7.539(7.8); 4.744(0.6); 4.729(0.7); 4.662(6.2); 4.646 |
| | | | (6.1); 3.509(0.7); 3.410(2.7); 3.392(7.8); 3.374(7.9); 3.357(2.9); 3.324(107.1); |
| | | | 2.957(3.4); 2.676(1.1); 2.671(1.4); 2.667(1.1); 2.511(91.7); 2.507(178.7); |
| | | | 2.502(231.3); 2.498(165.8); 2.494(80.6); 2.464(0.4); 2.334(1.1); 2.329(1.4); |
| | | | 2.325(1.0); 2.078(0.5); 2.061(1.3); 2.048(2.2); 2.031(1.4); 2.017(0.6); 1.755(0.3); |
| | | | 1.355(0.6); 1.234(0.8); 1.109(8.2); 1.091(16.0); 1.074(7.9); 1.044(1.1); 1.028(1.1); |
| | | | 0.913(0.3); 0.902(0.5); 0.875(11.9); 0.866(7.4); 0.855(6.0); 0.146(0.8); 0.008 |
| | | | (7.4); 0.000(179.2); −0.008(7.1); −0.150(0.8) |
| I-006 | 4.43 | 4.49 | Example I-006: ¹H-NMR(400.0 MHz, d₆-DMSO): δ = |
| | | | 11.012(4.2); 8.880(1.2); 8.864(2.4); 8.848(1.2); 8.579(5.1); 8.575(5.1); 8.317(0.8); |
| | | | 8.027(5.8); 8.024(5.6); 8.009(4.0); 7.989(6.2); 7.919(6.6); 7.899(4.3); 7.431(4.4); |
| | | | 7.416(1.2); 7.396(2.9); 7.378(4.3); 7.356(3.9); 7.351(3.7); 7.344(3.7); |
| | | | 7.336(1.9); 7.326(2.0); 7.282(0.5); 7.263(0.6); 7.242(0.4); 7.227(0.4); 6.673(0.4); |
| | | | 4.581(5.3); 4.565(5.2); 3.324(125.4); 3.182(0.8); 2.671(2.4); 2.589(0.4); 2.569(0.5); |
| | | | 2.541(1.5); 2.506(288.0); 2.502(366.4); 2.498(268.9); 2.329(2.3); 2.067(0.4); |
| | | | 2.050(1.2); 2.037(1.7); 2.022(1.1); 2.006(0.7); 1.754(16.0); 1.497(0.8); |
| | | | 1.251(0.4); 1.234(1.6); 0.872(9.7); 0.863(6.0); 0.853(4.9); 0.829(0.4); 0.146(0.8); |
| | | | 0.066(0.4); 0.008(7.2); 0.000(166.8); −0.008(7.7); −0.150(0.8) |
| I-007 | 2.75 | 3.19 | Example I-007: ¹H-NMR(400.0 MHz, d₆-DMSO): δ = |
| | | | 11.093(6.7); 8.930(2.0); 8.915(4.1); 8.900(2.0); 8.609(9.6); 8.605(9.4); 8.583(3.7); |
| | | | 8.581(3.7); 8.570(3.8); 8.569(3.6); 8.549(0.6); 8.316(1.0); 8.049(11.0); 8.045(10.7); |
| | | | 8.012(6.6); 7.991(10.4); 7.921(10.9); 7.900(7.0); 7.830(2.2); 7.826(2.1); |
| | | | 7.811(4.3); 7.806(4.2); 7.792(2.5); 7.787(2.5); 7.427(5.2); 7.408(4.6); 7.342(2.7); |
| | | | 7.328(2.8); 7.324(2.8); 7.311(2.5); 4.706(9.8); 4.691(9.7); 3.346(15.2); |
| | | | 3.169(0.9); 2.676(1.8); 2.671(2.5); 2.667(1.8); 2.541(1.9); 2.524(8.4); 2.511(151.5); |
| | | | 2.507(299.1); 2.502(387.9); 2.498(277.7); 2.493(133.3); 2.333(1.9); 2.329(2.5); |
| | | | 2.324(1.8); 2.320(0.9); 2.100(0.7); 2.086(1.7); 2.083(1.8); 2.075(2.2); |
| | | | 2.069(3.0); 2.056(1.9); 2.053(1.9); 2.039(0.8); 1.234(1.1); 0.885(16.0); 0.876 |
| | | | (10.0); 0.865(7.9); 0.842(0.6); 0.146(1.4); 0.021(0.7); 0.008(13.8); |
| | | | 0.000(339.2); −0.009(12.8); −0.150(1.4) |
| I-008 | 4.34 | 4.48 | Example I-008: ¹H-NMR(400.0 MHz, d₆-DMSO): δ = |
| | | | 11.012(5.4); 8.826(1.5); 8.810(3.2); 8.794(1.5); 8.578(7.0); 8.574(7.3); 8.317(0.4); |
| | | | 8.025(8.0); 8.021(8.1); 8.005(4.8); 7.984(7.6); 7.918(7.9); 7.897(5.0); 7.437(3.8); |
| | | | 7.431(1.9); 7.421(3.0); 7.415(16.0); 7.400(13.6); 7.394(3.0); 7.384(1.7); |
| | | | 7.378(3.6); 5.757(4.7); 4.569(6.8); 4.553(6.7); 3.352(9.9); 2.676(0.6); 2.672 |
| | | | (0.9); 2.667(0.7); 2.542(0.7); 2.525(3.0); 2.512(50.4); 2.507(101.1); 2.503(133.1); |
| | | | 2.498(96.6); 2.494(47.0); 2.334(0.6); 2.329(0.8); 2.325(0.6); 2.061(0.5); 2.047(1.2); |
| | | | 2.043(1.3); 2.037(1.0); 2.030(2.2); 2.017(1.3); 2.013(1.4); 1.999(0.6); |
| | | | 1.232(2.0); 0.871(11.6); 0.862(7.2); 0.851(5.8); 0.829(0.4); 0.146(0.5); 0.008 |
| | | | (5.2); 0.000(129.3); −0.009(5.1); −0.150(0.5) |
| I-009 | 3.77 | 3.82 | Example I-009: ¹H-NMR(400.0 MHz, d₆-DMSO): δ = |
| | | | 11.085(5.3); 8.839(1.5); 8.823(3.2); 8.806(1.5); 8.628(0.4); 8.612(7.5); 8.608(7.5); |
| | | | 8.316(1.0); 8.039(8.2); 8.036(8.1); 8.016(5.2); 7.996(7.8); 7.922(8.3); 7.901(5.3); |
| | | | 5.756(7.7); 4.263(0.8); 4.239(2.6); 4.221(2.8); 4.214(2.9); 4.197(2.6); |
| | | | 4.191(1.2); 4.173(0.9); 3.407(0.9); 3.360(30.7); 2.675(1.9); 2.671(2.6); |
| | | | 2.666(1.9); 2.541(2.3); 2.524(10.0); 2.511(156.8); 2.506(308.7); 2.502(403.4); 2.497 |
| | | | (290.0); 2.493(140.1); 2.333(1.8); 2.329(2.5); 2.324(1.8); 2.320(0.9); |
| | | | 2.104(0.5); 2.088(1.5); 2.074(2.3); 2.058(1.8); 2.043(0.7); 1.234(1.1); 0.887(16.0); |
| | | | 0.874(8.1); 0.869(7.5); 0.146(1.4); 0.008(14.2); 0.000(344.1); −0.008(14.4); −0.150(1.5) |
| I-010 | 4.89 | 5.00 | Example I-010: ¹H-NMR(400.0 MHz, d₆-DMSO): δ = |
| | | | 11.037(5.1); 8.813(1.5); 8.797(3.1); 8.782(1.5); 8.596(7.1); 8.592(6.9); 8.316(0.5); |
| | | | 8.029(8.1); 8.025(7.8); 8.008(4.7); 7.988(7.4); 7.920(7.7); 7.899(4.9); 7.669(4.8); |
| | | | 7.666(5.8); 7.472(0.7); 7.468(0.5); 7.450(9.8); 7.447(16.0); 7.427(0.5); |
| | | | 5.757(6.0); 4.631(6.8); 4.616(6.8); 3.324(67.5); 3.309(1.5); 2.676(0.9); 2.671 |
| | | | (1.3); 2.667(0.9); 2.566(0.4); 2.542(1.2); 2.525(4.6); 2.511(74.0); 2.507(146.8); |
| | | | 2.502(191.5); 2.498(136.4); 2.493(66.0); 2.334(0.8); 2.329(1.1); 2.325(0.8); |
| | | | 2.082(0.5); 2.065(1.3); 2.059(1.0); 2.051(2.2); 2.038(1.4); 2.020(0.6); 1.234(1.2); |
| | | | 0.879(11.5); 0.869(7.1); 0.858(5.7); 0.836(0.6); 0.000(0.6) |
| I-011 | 3.55 | 3.67 | Example I-011: ¹H-NMR(400.0 MHz, d₆-DMSO): δ = |
| | | | 10.990(6.3); 8.946(1.8); 8.931(3.7); 8.915(1.8); 8.568(8.5); 8.564(8.6); 8.437(5.6); |
| | | | 8.432(5.6); 8.316(1.1); 8.011(10.4); 8.007(12.0); 7.983(9.2); 7.916(9.6); 7.895(6.1); |
| | | | 7.862(3.4); 7.856(3.4); 7.842(3.8); 7.836(3.8); 7.527(6.5); 7.506(5.7); |
| | | | 4.592(8.0); 4.576(7.8); 3.322(138.4); 2.675(2.2); 2.671(2.9); 2.666(2.1); 2.541(1.8); |
| | | | 2.524(8.4); 2.511(173.9); 2.506(345.4); 2.502(447.6); 2.497(325.5); 2.493(158.5); |
| | | | 2.333(2.0); 2.329(2.7); 2.324(2.0); 2.075(0.8); 2.058(1.6); 2.051(1.3); |
| | | | 2.044(2.6); 2.030(1.7); 2.013(0.7); 1.235(0.5); 0.872(16.0); 0.862(8.7); 0.853(7.4); |
| | | | 0.146(1.5); 0.008(13.3); 0.000(370.2); −0.009(14.6); −0.150(1.6) |
| I-012 | 3.66 | 3.78 | Example I-012: ¹H-NMR(400.0 MHz, d₆-DMSO): δ = |
| | | | 11.089(5.4); 9.482(0.3); 8.584(7.4); 8.580(7.5); 8.496(1.7); 8.369(1.7); 8.354(3.2); |
| | | | 8.339(1.6); 8.316(0.4); 8.157(0.4); 8.137(0.4); 8.007(9.2); 8.003(11.2); 7.980(8.3); |
| | | | 7.916(8.8); 7.895(5.4); 5.757(0.5); 3.324(40.4); 3.306(0.6); 3.257(4.6); |
| | | | 3.241(7.2); 3.225(4.6); 2.677(0.6); 2.672(0.8); 2.668(0.6); 2.542(0.7); 2.512 |
| | | | (50.8); 2.507(99.2); 2.503(129.1); 2.499(94.0); 2.494(46.5); 2.334(0.5); 2.330(0.8); |
| | | | 2.325(0.6); 2.092(0.5); 2.076(1.5); 2.062(2.4); 2.046(1.7); 2.031(0.6); 1.232(0.9); |
| | | | 1.102(0.5); 1.096(0.6); 1.083(1.3); 1.077(1.1); 1.073(1.0); 1.065(1.9); |
| | | | 1.053(1.2); 1.045(1.3); 1.033(0.7); 1.027(0.5); 0.885(16.0); 0.873(8.2); |
| | | | 0.867(7.5); 0.843(0.5); 0.518(1.6); 0.507(5.0); 0.503(5.4); 0.493(3.0); 0.487(5.4); |
| | | | 0.483(5.1); 0.473(2.3); 0.460(0.4); 0.329(0.7); 0.317(0.7); 0.299(2.0); 0.286(6.1); |
| | | | 0.277(5.4); 0.274(6.1); 0.263(1.5); 0.146(0.4); 0.008(3.0); 0.000(74.0); −0.008(3.1) |
| I-013 | 3.37 | 3.37 | Example I-013: ¹H-NMR(400.0 MHz, d₆-DMSO): δ = |
| | | | 10.999(5.4); 8.558(6.8); 8.555(7.1); 8.467(0.4); 8.326(3.2); 8.315(3.5); 7.995(5.2); |
| | | | 7.975(8.5); 7.961(7.7); 7.958(7.7); 7.913(8.7); 7.892(5.3); 3.328(67.6); 2.947(0.4); |
| | | | 2.937(1.0); 2.928(1.3); 2.919(2.1); 2.908(2.2); 2.900(1.4); 2.890(1.1); |
| | | | 2.880(0.4); 2.676(0.8); 2.672(1.1); 2.667(0.8); 2.507(117.2); 2.503(155.7); 2.498(118.3); |
| | | | 2.334(0.7): 2.329(1.0); 2.325(0.8); 2.063(0.6); 2.047(1.8); 2.032(2.4); |
| | | | 2.017(2.0); 2.002(0.7); 1.234(0.6); 0.875(16.0); 0.861(9.6); 0.833(0.7); 0.827(0.6); |
| | | | 0.807(1.7); 0.794(4.2); 0.789(5.7); 0.777(5.4); 0.771(4.6); 0.760(2.1); |
| | | | 0.738(0.3); 0.720(0.3); 0.683(0.4); 0.673(0.4); 0.643(1.9); 0.632(5.4); |
| | | | 0.626(5.1); 0.622(4.8); 0.616(4.6); 0.604(1.5); 0.146(0.7); 0.008(6.1); |
| | | | 0.000(147.5); −0.008(8.7); −0.150(0.7) |
| I-014 | 3.81 | 3.88 | Example I-014: ¹H-NMR(400.0 MHz, d₆-DMSO): δ = |
| | | | 10.598(2.6); 8.944(0.7); 8.929(1.6); 8.913(0.8); 8.623(3.5); 8.620(3.4); 8.437(2.3); |
| | | | 8.431(2.3); 8.020(6.0); 8.017(4.8); 8.001(3.8); 7.924(3.9); 7.903(2.6); 7.860(1.5); |
| | | | 7.853(1.4); 7.839(1.7); 7.833(1.6); 7.526(2.8); 7.505(2.5); 5.757(3.0); |
| | | | 4.591(3.3); 4.576(3.3); 3.641(0.7); 3.624(0.3); 3.378(0.3); 3.362(0.8); 3.346(2.0); |
| | | | 3.327(19.1); 3.303(0.4); 2.831(0.4); 2.814(1.0); 2.797(1.5); 2.780(1.1); 2.763(0.4); |
| | | | 2.513(15.8); 2.509(30.0); 2.504(38.2); 2.500(27.3); 2.495(13.3); 1.520(1.5); |
| | | | 1.506(3.7); 1.500(2.0); 1.493(1.3); 1.475(0.5); 1.231(0.4); 1.137(16.0); |
| | | | 1.120(15.9); 1.075(0.4); 1.058(0.4); 1.045(1.8); 1.029(1.8); 0.008(2.4); |
| | | | 0.000(41.9); −0.008(1.7) |
| I-015 | 3.85 | 3.91 | Example I-015: ¹H-NMR(400.0 MHz, d₆-DMSO): δ = |
| | | | 10.615(2.7); 8.975(0.7); 8.959(1.6); 8.943(0.8); 8.634(3.5); 8.630(3.5); 8.033(4.2); |
| | | | 8.029(5.1); 8.005(3.8); 7.927(3.9); 7.906(2.6); 7.840(4.1); 7.819(4.9); 7.557(4.2); |
| | | | 7.536(3.6); 5.757(2.9); 4.661(3.1); 4.645(3.1); 3.340(5.7); 2.834(0.4); |
| | | | 2.817(1.0); 2.800(1.4); 2.783(1.1); 2.766(0.5); 2.526(0.6); 2.513(13.1); 2.509(26.2); |
| | | | 2.504(34.1); 2.500(24.7); 2.495(12.0); 1.140(16.0); 1.123(16.0); 1.108(0.5); |
| | | | 1.103(0.4); 1.045(0.9); 1.029(0.9); 0.008(0.6); 0.000(14.5); −0.008(0.5) |
| I-016 | 3.23 | 3.30 | Example I-016: ¹H-NMR(400.0 MHz, d₆-DMSO): δ = |
| | | | 11.044(4.3); 8.566(6.1); 8.562(5.9); 8.339(1.3); 8.324(2.4); 8.316(1.3); 8.309(1.3); |
| | | | 7.997(4.4); 7.987(7.5); 7.983(8.4); 7.977(7.0); 7.915(7.3); 7.894(4.5); 7.861(0.5); |
| | | | 7.842(0.4); 7.824(0.4); 7.793(0.6); 7.772(0.5); 7.749(0.5); 7.719(0.5); |
| | | | 7.685(0.5); 7.640(0.6); 7.614(0.6); 7.593(0.6); 7.580(0.4); 7.562(0.4); 7.521(0.4); |
| | | | 7.496(0.3); 7.488(0.4); 7.468(0.5); 7.447(0.4); 7.417(0.3); 7.404(0.4); 7.384 |
| | | | (0.3); 5.756(3.4); 3.925(0.6); 3.419(1.1); 3.401(3.5); 3.385(4.4); 3.368(3.8); |
| | | | 3.350(2.4); 3.325(191.6); 3.282(0.4); 3.182(0.5); 2.676(1.2); 2.671(1.5); 2.667(1.1); |
| | | | 2.541(1.0); 2.511(97.8); 2.507(188.0); 2.502(240.5); 2.498(170.7); 2.494(81.2); |
| | | | 2.461(0.5); 2.334(1.2); 2.329(1.6); 2.325(1.2); 2.083(0.5); 2.067(1.3); |
| | | | 2.052(1.9); 2.037(1.5); 2.021(0.6); 1.882(0.4); 1.755(2.9); 1.694(0.4); 1.505(0.5); |
| | | | 1.234(1.7); 1.217(0.5); 1.194(7.7); 1.176(16.0); 1.158(7.7); 1.131(0.4); 1.078 |
| | | | (0.4); 1.064(0.4); 1.028(0.6); 1.012(0.7); 0.998(0.5); 0.980(0.5); 0.962(0.3); |
| | | | 0.881(13.5); 0.868(7.1); 0.863(6.6); 0.000(1.0) |
| I-017 | 3.13 | 3.15 | Example I-017: ¹H-NMR(400.0 MHz, d₆-DMSO): δ = |
| | | | 10.685(3.0); 8.968(0.9); 8.952(1.9); 8.936(0.9); 8.560(2.0); 8.025(5.0); 8.021(4.9); |
| | | | 8.008(2.9); 7.987(5.0); 7.932(5.2); 7.911(3.0); 7.838(5.3); 7.818(6.1); 7.554(5.2); |
| | | | 7.533(4.5); 5.757(4.0); 4.655(3.9); 4.640(3.9); 3.327(30.0); 2.672(0.4); |
| | | | 2.526(0.9); 2.512(23.9); 2.508(48.0); 2.503(62.4); 2.499(44.5); 2.494(21.1); |
| | | | 2.330(0.4); 2.325(0.3); 2.162(16.0); 0.008(0.7); 0.000(20.7); −0.009(0.7) |
| I-018 | 3.68 | 2.65 | Example I-018: ¹H-NMR(400.0 MHz, d₆-DMSO): δ = |
| | | | 11.065(2.3); 8.576(3.7); 8.572(3.6); 8.485(0.6); 8.099(1.3); 8.079(1.3); 7.996(2.5); |
| | | | 7.992(5.1); 7.988(4.3); 7.976(3.6); 7.932(0.3); 7.914(3.9); 7.893(2.3); 4.153(0.6); |
| | | | 4.136(0.8); 4.133(0.7); 4.120(0.7); 4.116(0.8); 4.099(0.6); 3.324(30.1); |
| | | | 2.672(0.4); 2.526(0.9); 2.521(1.5); 2.512(21.9); 2.508(44.6); 2.503(58.3); |
| | | | 2.499(40.6); 2.494(18.6); 2.330(0.4); 2.064(0.7); 2.049(1.1); 2.033(0.8); |
| | | | 2.018(0.4); 1.317(1.3); 1.301(1.3); 1.239(16.0); 1.223(15.9); 1.212(1.1); 0.883(7.5); |
| | | | 0.870(3.9); 0.866(3.6); 0.008(0.6); 0.000(18.0); −0.009(0.5) |
| I-019 | 4.33 | 4.40 | Example I-019: ¹H-NMR (601.6 MHz, d₆-DMSO): δ = |
| | | | 11.122(5.8); 10.742(6.8); 8.643(6.4); 8.641(6.4); 8.314(0.3); 8.096(8.3); 8.094 |
| | | | (8.3); 8.053(8.6); 8.049(3.4); 8.041(7.5); 8.038(11.9); 8.029(6.9); 7.932(7.1); |
| | | | 7.918(5.7); 7.901(9.9); 7.898(3.1); 7.889(2.8); 7.886(8.1); 3.342(59.4); |
| | | | 3.338(67.9); 3.336(64.9); 3.332(72.1); 3.329(82.0); 3.323(98.4); 3.322(136.8); |
| | | | 3.321(123.6); 2.945(1.9); 2.785(1.3); 2.617(1.2); 2.614(1.7); 2.611(1.2); 2.542(0.5); |
| | | | 2.523(2.4); 2.520(3.0); 2.517(2.8); 2.508(78.4); 2.505(175.6); 2.502(251.2); |
| | | | 2.499(185.0); 2.496(89.5); 2.389(1.2); 2.386(1.7); 2.383(1.2); 2.104(0.4); |
| | | | 2.093(1.3); 2.083(1.9); 2.073(1.6); 2.063(0.5); 1.958(1.5); 1.755(1.8); 0.907(16.0); |
| | | | 0.898(7.7); 0.894(6.9); 0.005(0.9); 0.000(36.5); −0.006(1.6) |
| I-020 | 3.54 | 3.60 | Example I-020: ¹H-NMR(400.0 MHz, d₆-DMSO): δ = |
| | | | 10.956(7.3); 8.948(2.0); 8.932(4.2); 8.916(2.1); 8.522(8.9); 8.519(8.8); 8.490(0.4); |
| | | | 8.316(0.4); 7.969(9.8); 7.966(9.4); 7.949(0.4); 7.838(10.6); 7.817(14.1); 7.813(12.9); |
| | | | 7.791(12.9); 7.758(0.3); 7.619(12.9); 7.598(10.4); 7.554(11.2); 7.534(9.7); |
| | | | 7.509(0.6); 7.476(0.4); 7.406(0.4); 7.384(0.4); 7.372(0.4); 7.329(0.3); |
| | | | 5.756(11.1); 4.655(8.4); 4.639(8.4); 3.323(47.5); 2.671(1.1); 2.541(0.6); 2.507 |
| | | | (133.8); 2.502(170.8); 2.498(128.2); 2.333(1.0); 2.329(1.3); 2.325(1.0); 2.068(0.8); |
| | | | 2.051(1.9); 2.037(3.1); 2.023(2.1); 2.006(0.9); 1.234(1.0); 1.039(0.4); 1.030(0.5); |
| | | | 1.020(0.5); 0.992(0.4); 0.981(0.4); 0.964(0.4); 0.895(0.9); 0.867(16.0); |
| | | | 0.858(10.6); 0.847(8.3); 0.008(0.6); 0.000(12.9) |
| I-021 | 5.08 | 4.96 | Example I-021: ¹H-NMR(400.0 MHz, d₆-DMSO): δ = |
| | | | 11.112(6.0); 10.457(7.0); 8.629(7.5); 8.625(7.3); 8.315(1.3); 8.079(8.0); 8.076 |
| | | | (7.8); 8.040(5.5); 8.020(8.1); 7.951(0.6); 7.933(8.4); 7.912(5.7); 7.887(8.9); |
| | | | 7.870(3.4); 7.865(10.0); 7.857(1.0); 7.491(10.2); 7.487(3.4); 7.474(3.3); |
| | | | 7.469(9.2); 7.462(1.0); 3.321(202.2); 2.675(2.7); 2.671(3.6); 2.666(2.7); 2.593(0.4); |
| | | | 2.541(2.6); 2.524(10.9); 2.510(205.7); 2.506(403.1); 2.502(526.8); 2.497(388.0); |
| | | | 2.333(2.6); 2.328(3.5); 2.324(2.6); 2.105(0.6); 2.090(1.8); 2.074(5.7); 2.059(2.0); |
| | | | 2.044(0.6); 0.907(16.0); 0.892(9.5); 0.008(1.3); 0.000(37.6); −0.008(1.7) |
| I-022 | 4.29 | 4.34 | Example I-022: ¹H-NMR(400.0 MHz, d₆-DMSO): δ = |
| | | | 11.076(5.6); 10.725(6.5); 8.676(0.4); 8.589(7.3); 8.585(7.3); 8.316(1.6); 8.143 |
| | | | (0.4); 8.051(8.2); 8.046(3.8); 8.042(8.7); 8.038(8.6); 8.034(4.0); 8.029(10.8); |
| | | | 7.968(0.3); 7.946(0.3); 7.900(10.8); 7.896(3.5); 7.878(8.3); 7.855(8.8); 7.850(3.2); |
| | | | 7.838(3.6); 7.833(10.8); 7.827(1.5); 7.683(0.3); 7.673(0.3); 7.657(0.6); 7.636(11.1); |
| | | | 7.632(3.5); 7.620(3.2); 7.615(9.1); 7.578(0.4); 7.564(0.4); 7.538(0.4); |
| | | | 7.529(0.4); 7.508(0.5); 7.482(0.5); 7.470(0.4); 7.463(0.5); 7.439(0.5); 7.429(0.5); |
| | | | 7.412(0.5); 7.401(0.5); 7.387(0.5); 7.377(0.5); 7.363(0.5); 7.326(0.5); 7.318 |
| | | | (0.5); 7.302(0.5); 7.288(0.5); 7.282(0.5); 7.264(0.5); 7.243(0.4); 7.231(0.4); |
| | | | 7.202(0.3); 7.169(0.4); 7.150(0.4); 5.756(5.6); 3.321(275.7); 2.679(1.5); 2.675(3.2); |
| | | | 2.671(4.5); 2.666(3.3); 2.662(1.5); 2.541(1.8); 2.524(9.8); 2.510(253.1); 2.506(514.5); |
| | | | 2.502(682.7); 2.497(496.5); 2.493(241.0); 2.425(0.9); 2.418(0.9); |
| | | | 2.381(0.6); 2.337(1.9); 2.333(3.6); 2.328(4.8); 2.324(3.6); 2.319(1.9); 2.288(0.4); |
| | | | 2.180(0.3); 2.170(0.3); 2.132(0.3); 2.116(0.3); 2.104(0.8); 2.089(1.9); 2.074 |
| | | | (2.5); 2.059(2.2); 2.043(0.9); 1.753(2.0); 1.298(1.2); 1.259(1.7); 1.234(2.7); |
| | | | 1.170(0.4); 1.152(0.4); 1.145(0.4); 1.129(0.4); 1.104(0.4); 1.056(0.7); 1.044(0.7); |
| | | | 1.029(0.6); 1.019(0.6); 1.006(0.5); 0.970(0.5); 0.940(0.7); 0.933(0.8); 0.901(16.0); |
| | | | 0.887(9.4); 0.853(1.2); 0.836(0.8); 0.008(1.7); 0.000(54.9); −0.008(2.0) |
| I-023 | 4.66 | 4.77 | Example I-023: ¹H-NMR(400.0 MHz, d₆-DMSO): δ = |
| | | | 13.176(0.6); 10.951(9.8); 9.123(2.7); 9.107(5.1); 9.091(2.6); 8.650(10.7); |
| | | | 8.316(0.7); 8.172(0.4); 8.160(0.5); 8.121(11.2); 8.089(13.1); 8.078(11.8); 8.068(16.0); |
| | | | 8.059(13.3); 8.011(0.3); 7.951(13.3); 7.931(10.3); 7.879(0.4); 7.843(11.3); |
| | | | 7.823(12.8); 7.764(0.3); 7.746(0.3); 7.658(12.6); 7.637(11.5); 7.567(12.7); |
| | | | 7.547(10.8); 4.682(9.9); 4.667(9.8); 3.324(270.3); 2.671(3.4); 2.502(480.1); |
| | | | 2.368(0.5); 2.329(3.4); 2.075(0.4); 1.755(0.4); 1.235(1.0); 0.146(0.7); |
| | | | 0.000(135.5); −0.150(0.8) |
| I-024 | 3.47 | 3.53 | Example I-024: ¹H-NMR(400.0 MHz, d₆-DMSO): δ = |
| | | | 10.938(5.7); 8.922(1.6); 8.906(3.3); 8.890(1.5); 8.511(7.4); 8.507(7.3); 8.472(0.5); |
| | | | 8.433(4.7); 8.427(4.6); 8.363(0.3); 8.359(0.4); 8.316(0.6); 8.150(0.4); 8.146(0.4); |
| | | | 7.966(0.7); 7.956(8.2); 7.952(7.8); 7.857(3.0); 7.851(2.9); 7.836(3.6); |
| | | | 7.830(3.5); 7.816(2.4); 7.808(8.8); 7.803(3.2); 7.791(3.6); 7.786(10.5); 7.780(1.3); |
| | | | 7.631(0.8); 7.623(1.7); 7.616(11.0); 7.611(3.6); 7.599(3.4); 7.595(9.9); 7.582(2.9); |
| | | | 7.578(1.5); 7.524(5.7); 7.503(5.1); 7.493(0.5); 7.485(1.0); 7.475(1.1); |
| | | | 7.466(1.0); 7.457(0.7); 7.453(0.7); 7.444(0.6); 5.756(2.2); 4.584(6.8); 4.569(6.7); |
| | | | 3.862(16.0); 3.835(0.6); 3.322(109.9); 2.676(1.3); 2.671(1.7); 2.667(1.3); |
| | | | 2.541(1.3); 2.524(5.4); 2.511(99.8); 2.507(194.0); 2.502(251.0); 2.498(179.9); |
| | | | 2.493(85.9); 2.338(0.6); 2.333(1.2); 2.329(1.6); 2.324(1.2); 2.065(0.5); 2.048(1.4); |
| | | | 2.041(1.1); 2.034(2.3); 2.020(1.5); 2.003(0.7); 1.754(1.2); 1.233(0.8); 1.043(0.6); |
| | | | 1.032(0.5); 1.028(0.7); 1.014(0.5); 0.913(0.4); 0.906(0.6); 0.896(0.6); |
| | | | 0.887(0.9); 0.865(13.3); 0.854(7.6); 0.845(6.2); 0.822(0.5); 0.146(0.7); |
| | | | 0.008(7.3); 0.000(167.6); −0.009(6.0); −0.150(0.7) |
| I-025 | 3.01 | 3.05 | Example I-025: ¹H-NMR(400.0 MHz, d₆-DMSO): δ = |
| | | | 11.038(5.5); 9.014(1.6); 8.999(3.4); 8.984(1.7); 8.594(7.6); 8.590(7.7); 8.023(11.2); |
| | | | 8.019(13.0); 7.999(8.0); 7.941(0.3); 7.923(8.4); 7.902(5.6); 5.757(9.0); |
| | | | 4.423(9.1); 4.408(9.1); 3.652(0.7); 3.640(1.4); 3.625(0.9); 3.328(123.8); |
| | | | 2.677(0.6); 2.672(0.8); 2.668(0.6); 2.526(1.6); 2.521(2.7); 2.512(46.6); 2.508(95.3); |
| | | | 2.503(125.8); 2.499(90.8); 2.494(43.7); 2.334(0.7); 2.330(0.9); 2.325(0.7); |
| | | | 2.114(0.6); 2.098(1.5); 2.090(1.1); 2.083(2.3); 2.067(1.7); 2.052(0.8); 1.233(0.4); |
| | | | 1.043(0.4); 1.031(0.3); 0.941(0.3); 0.933(0.4); 0.921(0.4); 0.912(0.6); |
| | | | 0.886(16.0); 0.874(8.7); 0.868(7.6); 0.146(0.8); 0.008(6.2); |
| | | | 0.000(178.0); −0.009(6.7); −0.025(0.4); −0.028(0.4); −0.150(0.8) |
| I-026 | 3.79 | 3.87 | Example I-026: ¹H-NMR(400.0 MHz, d₆-DMSO): δ = |
| | | | 11.004(6.9); 9.039(1.9); 9.024(4.1); 9.008(1.9); 8.580(9.7); 8.577(9.8); 8.317(0.6); |
| | | | 8.021(11.4); 8.017(11.5); 8.009(6.2); 7.989(9.8); 7.935(4.0); 7.919(12.9); |
| | | | 7.898(10.3); 7.511(3.7); 7.508(3.8); 7.485(3.7); 7.482(3.8); 7.406(4.4); 7.403(4.2); |
| | | | 7.386(4.0); 7.383(3.9); 4.669(7.6); 4.653(7.7); 3.325(142.4); 2.996(0.7); |
| | | | 2.712(0.5); 2.681(0.5); 2.676(1.1); 2.672(1.5); 2.667(1.1); 2.663(0.5); |
| | | | 2.542(149.5); 2.525(3.4); 2.520(5.4); 2.512(84.1); 2.507(174.3); 2.503(231.6); |
| | | | 2.498(164.1); 2.493(76.6); 2.368(0.6); 2.338(0.5); 2.334(1.1); 2.329(1.5); 2.325(1.1); |
| | | | 2.320(0.5); 2.092(0.7); 2.079(1.5); 2.075(1.7); 2.069(1.3); 2.062(2.9); 2.048(1.7); |
| | | | 2.045(1.8); 2.031(0.8); 0.879(16.0); 0.869(8.8); 0.859(7.3); 0.852(2.5); 0.836 |
| | | | (0.5); 0.146(1.4); 0.008(11.4); 0.000(334.9); −0.009(11.0); −0.150(1.4) |
| I-027 | 4.50 | 4.62 | Example I-027: ¹H-NMR(400.0 MHz, d₆-DMSO): δ = |
| | | | 11.012(7.8); 8.944(2.1); 8.929(4.6); 8.913(2.2); 8.584(10.3); 8.580(10.8); |
| | | | 8.031(11.5); 8.027(11.7); 8.009(6.9); 7.988(11.0); 7.920(11.4); 7.899(7.2); |
| | | | 7.740(8.0); 7.720(10.5); 7.594(9.5); 7.574(7.4); 4.672(7.7); 4.656(7.7); 3.329(81.4); |
| | | | 2.997(0.4); 2.677(0.6); 2.673(0.8); 2.668(0.6); 2.543(40.2); 2.526(2.2); 2.512(46.4); |
| | | | 2.508(95.3); 2.503(127.0); 2.499(93.0); 2.495(45.6); 2.370(0.5); 2.339(0.3); |
| | | | 2.335(0.6); 2.330(0.9); 2.326(0.6); 2.070(0.8); 2.056(1.7); 2.052(1.9); 2.046(1.5); |
| | | | 2.039(3.3); 2.032(1.4); 2.026(2.0); 2.022(2.0); 2.008(0.9); 0.874(16.0); |
| | | | 0.866(10.4); 0.854(8.2); 0.832(0.6); 0.146(0.7); 0.008(6.4); |
| | | | 0.000(173.9); −0.009(6.9); −0.024(0.3); −0.150(0.8) |
| I-028 | 4.18 | 4.27 | Example I-028: ¹H-NMR(400.0 MHz, d₆-DMSO): δ = |
| | | | 11.010(0.5); 9.527(2.2); 9.511(4.8); 9.495(2.3); 8.581(0.7); 8.577(0.7); 8.550(0.7); |
| | | | 8.425(10.8); 8.421(11.5); 8.317(3.1); 8.236(11.5); 8.232(11.0); 8.175(0.5); |
| | | | 8.153(8.3); 8.132(10.0); 8.021(0.8); 8.018(0.8); 8.008(0.6); 7.988(0.8); 7.934 |
| | | | (11.1); 7.911(10.8); 7.891(6.0); 7.873(4.5); 7.811(0.3); 7.510(0.5); 7.468(4.8); |
| | | | 7.442(4.8); 7.405(0.6); 7.383(5.5); 7.363(4.7); 5.757(6.2); 5.187(0.4); 4.668(0.7); |
| | | | 4.653(0.7); 4.615(8.4); 4.599(8.4); 3.709(0.5); 3.694(0.4); 3.405(0.4); 3.377 |
| | | | (0.4); 3.328(811.9); 3.283(0.4); 2.676(4.6); 2.671(6.5); 2.667(4.9); 2.579(0.5); |
| | | | 2.564(0.8); 2.525(15.4); 2.520(23.7); 2.511(352.4); 2.507(733.6); 2.502(980.5); |
| | | | 2.498(720.9); 2.493(358.9); 2.420(0.6); 2.366(0.4); 2.334(4.7); 2.329(6.5); |
| | | | 2.325(4.9); 2.246(0.3); 2.203(0.4); 2.181(0.4); 2.067(1.7); 2.056(3.6); 2.048(4.4); |
| | | | 2.037(7.4); 2.025(4.6); 2.017(4.0); 2.005(2.0); 1.909(0.3); 1.754(0.9); 1.235 |
| | | | (2.4); 1.112(0.4); 1.061(3.9); 1.051(11.0); 1.042(16.0); 1.032(12.8); 1.024(5.7); |
| | | | 0.997(1.1); 0.987(1.0); 0.973(0.9); 0.953(1.1); 0.935(4.8); 0.927(11.6); 0.917 |
| | | | (10.7); 0.908(13.2); 0.899(9.4); 0.889(4.1); 0.879(2.2); 0.868(1.8); 0.838(0.7); |
| | | | 0.146(0.6); 0.008(3.5); 0.000(125.1); −0.008(5.0); −0.149(0.5) |
| I-029 | 3.21 | 3.28 | Example I-029: ¹H-NMR(400.0 MHz, d₆-DMSO): δ = |
| | | | 10.939(5.8); 10.010(0.5); 8.947(1.6); 8.931(3.4); 8.915(1.6); 8.515(7.4); 8.511 |
| | | | (7.3); 8.487(2.6); 8.316(0.9); 8.028(0.9); 8.011(0.5); 8.006(0.9); 7.966(8.2); |
| | | | 7.962(8.0); 7.852(9.0); 7.847(3.9); 7.839(11.2); 7.830(10.9); 7.823(5.8); 7.818(12.5); |
| | | | 7.803(0.8); 7.756(0.4); 7.555(9.9); 7.537(8.2); 7.534(9.3); 7.449(0.3); 7.429(0.4); |
| | | | 7.382(1.0); 7.371(1.3); 7.353(14.3); 7.330(8.2); 7.214(0.3); 7.198(0.6); |
| | | | 7.169(3.9); 7.147(0.4); 5.757(16.0); 4.735(0.9); 4.719(1.0); 4.654(6.3); 4.639 |
| | | | (6.3); 3.966(0.4); 3.325(170.0); 2.676(1.3); 2.671(1.8); 2.667(1.3); 2.542(1.0); |
| | | | 2.524(5.5); 2.511(113.1); 2.507(218.5); 2.502(279.2); 2.498(196.7); 2.493(91.9); |
| | | | 2.333(1.4); 2.329(1.8); 2.325(1.3); 2.068(0.6); 2.055(1.3); 2.050(1.4); 2.044(1.1); |
| | | | 2.037(2.3); 2.024(1.4); 2.020(1.4); 2.006(0.7); 1.233(0.7); 0.866(11.3); |
| | | | 0.858(7.6); 0.846(5.9); 0.824(0.6); 0.008(3.0); 0.000(63.2); −0.008(2.2) |
| I-030 | 2.83 | 2.94 | Example I-030: ¹H-NMR(400.0 MHz, d₆-DMSO): δ = |
| | | | 10.930(5.4); 9.272(0.3); 9.265(0.4); 8.495(6.9); 8.492(7.3); 8.404(1.3); 8.300(3.3); |
| | | | 8.289(3.3); 7.987(0.5); 7.965(0.5); 7.904(7.5); 7.901(7.8); 7.840(8.4); 7.818(9.5); |
| | | | 7.536(2.9); 7.375(0.5); 7.348(10.0); 7.326(7.9); 7.241(0.4); 7.212(0.4); |
| | | | 7.191(0.4); 7.167(3.1); 5.757(5.5); 3.798(0.4); 3.771(0.4); 3.640(0.4); 3.329(61.0); |
| | | | 2.939(0.4); 2.929(1.0); 2.919(1.4); 2.911(2.2); 2.900(2.2); 2.891(1.4); 2.882(1.1); |
| | | | 2.872(0.5); 2.677(0.4); 2.673(0.5); 2.669(0.4); 2.508(57.2); 2.503(75.4); |
| | | | 2.499(56.8); 2.369(1.8); 2.335(0.4); 2.330(0.5); 2.054(0.6); 2.038(1.7); 2.023(2.4); |
| | | | 2.008(1.9); 1.992(0.7); 1.018(0.3); 0.910(0.4); 0.868(16.0); 0.855(8.8); |
| | | | 0.851(8.4); 0.826(1.0); 0.821(1.0); 0.803(2.7); 0.790(4.7); 0.785(6.2); 0.772(5.6); |
| | | | 0.767(4.8); 0.755(2.0); 0.734(0.5); 0.716(0.6); 0.704(0.6); 0.699(0.4); 0.688(0.8); |
| | | | 0.678(0.8); 0.636(2.0); 0.625(5.7); 0.619(5.4); 0.616(5.1); 0.610(4.8); |
| | | | 0.597(1.5); 0.146(0.5); 0.007(4.8); 0.000(99.6); −0.008(5.0); −0.150(0.5) |
| I-031 | 4.06 | 4.17 | Example I-031: ¹H-NMR(400.0 MHz, d₆-DMSO): δ = |
| | | | 11.086(5.4); 11.070(0.3); 9.047(0.4); 9.017(0.4); 8.582(7.0); 8.579(7.3); 8.493 |
| | | | (1.6); 8.316(3.2); 8.224(3.1); 8.202(3.2); 8.183(0.3); 8.176(0.5); 8.156(0.8); |
| | | | 8.142(0.4); 8.136(0.6); 8.061(0.4); 8.036(0.4); 7.999(12.6); 7.996(10.7); 7.979(8.5); |
| | | | 7.915(9.0); 7.894(5.3); 7.840(0.3); 7.737(0.3); 7.715(0.4); 7.693(0.4); 7.646(0.3); |
| | | | 7.604(0.4); 7.592(0.4); 7.492(0.3); 7.451(0.4); 7.322(0.3); 7.297(0.3); 7.217(0.4); |
| | | | 7.214(0.4); 7.179(0.3); 3.983(0.9); 3.925(0.4); 3.711(0.4); 3.702(0.4); |
| | | | 3.695(0.4); 3.650(0.3); 3.625(0.7); 3.613(0.4); 3.592(0.4); 3.571(0.3); 3.505(0.7); |
| | | | 3.493(1.4); 3.472(2.4); 3.455(2.5); 3.433(1.6); 3.418(0.8); 3.391(1.4); 3.327(1842.2); |
| | | | 3.240(0.5); 3.236(0.4); 2.921(0.3); 2.675(6.6); 2.671(9.2); 2.667(7.0); |
| | | | 2.506(1136.2); 2.502(1492.6); 2.497(1116.4); 2.421(1.7); 2.411(1.6); 2.394(1.2); |
| | | | 2.379(1.1); 2.333(7.4); 2.329(9.9); 2.324(7.7); 2.295(0.8); 2.281(0.7); 2.230(0.6); |
| | | | 2.196(0.5); 2.173(1.2); 2.152(0.5); 2.136(0.4); 2.117(0.4); 2.100(0.4); |
| | | | 2.085(0.8); 2.072(2.0); 2.055(2.7); 2.040(2.2); 2.024(0.8); 1.962(0.4); 1.947(0.4); |
| | | | 1.932(0.4); 1.842(0.4); 1.581(0.4); 1.519(0.5); 1.503(0.4); 1.466(0.3); 1.386(2.0); |
| | | | 1.369(2.0); 1.357(0.5); 1.341(0.4); 1.326(0.5); 1.311(0.7); 1.282(14.2); |
| | | | 1.265(14.2); 1.234(4.4); 1.197(0.8); 1.171(0.7); 1.159(0.7); 1.150(0.7); 1.124 |
| | | | (0.5); 1.093(0.6); 1.057(1.5); 1.044(2.3); 1.034(1.9); 1.024(2.4); 1.012(1.6); |
| | | | 1.003(1.3); 0.990(0.9); 0.943(0.6); 0.936(0.7); 0.885(16.0); 0.871(9.7); 0.804(0.5); |
| | | | 0.785(0.5); 0.777(0.4); 0.766(0.4); 0.708(0.5); 0.693(0.3); 0.553(0.4); 0.532 |
| | | | (1.1); 0.523(1.9); 0.511(2.0); 0.501(2.2); 0.488(1.4); 0.481(1.2); 0.470(1.3); |
| | | | 0.459(1.7); 0.447(2.1); 0.438(2.2); 0.428(2.0); 0.406(0.9); 0.380(0.5); 0.357(0.9); |
| | | | 0.347(1.2); 0.336(1.8); 0.324(2.8); 0.309(2.8); 0.301(2.9); 0.297(3.0); |
| | | | 0.289(2.6); 0.277(1.7); 0.266(1.3); 0.258(0.7); 0.000(35.0) |
| I-032 | 3.98 | 4.06 | Example I-032: ¹H-NMR(400.0 MHz, d₆-DMSO): δ = |
| | | | 11.038(2.4); 8.578(3.2); 8.574(3.4); 8.332(0.7); 8.316(1.4); 8.300(0.7); 7.996(5.1); |
| | | | 7.992(4.4); 7.978(3.8); 7.915(3.9); 7.894(2.4); 5.758(1.1); 3.345(0.5); 3.329(18.2); |
| | | | 3.224(2.0); 3.207(3.5); 3.191(2.0); 2.527(0.6); 2.513(12.1); 2.509(24.8); |
| | | | 2.505(33.0); 2.500(24.3); 2.496(12.1); 2.065(0.7); 2.050(1.1); 2.035(0.8); |
| | | | 2.019(0.3); 1.901(0.4); 1.884(0.9); 1.867(1.1); 1.851(0.9); 1.834(0.5); |
| | | | 1.506(0.7); 0.946(16.0); 0.929(15.4); 0.888(7.5); 0.875(4.0); 0.871(3.7) |
| I-033 | 3.80 | 3.88 | Example I-033: ¹H-NMR(400.0 MHz, d₆-DMSO): δ = |
| | | | 11.049(5.4); 8.566(6.9); 8.562(7.2); 8.500(2.9); 8.479(3.0); 8.465(1.4); 8.316(0.9); |
| | | | 8.111(0.4); 7.993(4.8); 7.972(8.2); 7.958(7.8); 7.954(7.9); 7.912(8.7); 7.891(5.3); |
| | | | 4.510(0.3); 4.489(1.1); 4.468(2.1); 4.448(2.1); 4.427(1.1); 3.324(289.6); |
| | | | 2.675(1.9); 2.671(2.6); 2.667(1.9); 2.524(6.7); 2.506(304.3); 2.502(399.9); |
| | | | 2.498(294.2); 2.333(2.2); 2.329(3.1); 2.324(2.8); 2.296(3.0); 2.287(2.8); 2.277 |
| | | | (3.1); 2.123(0.9); 2.100(2.3); 2.069(4.6); 2.053(4.6); 2.038(2.5); 2.023(1.4); |
| | | | 1.785(0.4); 1.759(1.4); 1.745(2.6); 1.735(2.7); 1.719(3.3); 1.701(2.0); |
| | | | 1.693(1.7); 1.675(0.8); 1.235(0.6); 0.885(16.0); 0.870(10.4); 0.000(1.8) |
| I-034 | 2.72 | 2.73 | Example I-034: ¹H-NMR(400.0 MHz, d₆-DMSO): δ = |
| | | | 11.128(0.3); 11.023(0.5); 10.954(0.8); 8.577(0.5); 8.559(0.3); 8.493(1.1); 8.456(1.2); |
| | | | 8.362(3.2); 8.316(2.7); 8.005(0.6); 7.998(0.6); 7.978(0.9); 7.960(0.7); 7.916(1.1); |
| | | | 7.869(12.9); 7.763(1.8); 7.664(0.4); 7.562(1.7); 7.469(0.4); 7.427(0.4); 7.364(1.7); |
| | | | 5.756(4.7); 3.437(23.3); 3.387(1.1); 3.323(384.6); 3.260(0.3); 3.026(1.4); 2.959(1.7); |
| | | | 2.675(4.2); 2.671(5.8); 2.667(4.3); 2.524(16.0); 2.511(334.7); 2.506(679.0); |
| | | | 2.502(898.0); 2.497(655.2); 2.493(324.3); 2.418(0.5); 2.333(4.1); 2.329(5.8); |
| | | | 2.324(4.3); 2.030(0.8); 2.013(1.9); 1.997(2.4); 1.985(1.9); 1.506(0.4); 1.235(1.3); |
| | | | 0.883(1.1); 0.864(1.5); 0.832(15.9); 0.817(16.0); 0.146(5.5); 0.008(45.3); |
| | | | 0.000(1247.8); −0.009(54.3); −0.086(0.5); −0.097(0.5); −0.110(0.3); −0.126 |
| | | | (0.3); −0.150(5.9) |
| I-035 | 3.39 | 3.48 | Example I-035: ¹H-NMR(400.0 MHz, d₆-DMSO): δ = |
| | | | 10.945(5.0); 9.271(0.4); 9.263(0.4); 8.497(7.2); 8.493(7.0); 8.398(2.0); 8.317(0.7); |
| | | | 8.299(3.0); 8.287(3.0); 7.939(0.8); 7.919(9.5); 7.898(16.0); 7.893(8.7); 7.707(0.8); |
| | | | 7.685(0.7); 7.571(9.9); 7.567(3.2); 7.554(3.1); 7.550(8.7); 7.544(1.0); |
| | | | 3.361(0.3); 3.325(130.6); 2.936(0.4); 2.926(1.0); 2.916(1.3); 2.908(2.1); 2.897(2.1); |
| | | | 2.889(1.3); 2.879(1.0); 2.868(0.5); 2.848(0.3); 2.839(0.4); 2.676(1.2); 2.671(1.6); |
| | | | 2.667(1.2); 2.541(0.9); 2.525(4.4); 2.520(6.8); 2.511(84.7); 2.507(171.5); |
| | | | 2.502(224.7); 2.498(159.4); 2.493(74.4); 2.338(0.5); 2.334(1.0); 2.329(1.4); |
| | | | 2.324(1.0); 2.320(0.5); 2.050(0.5); 2.034(1.5); 2.019(2.1); 2.004(1.7); 1.988(0.6); |
| | | | 1.505(0.4); 0.866(15.3); 0.852(8.1); 0.849(7.7); 0.824(0.9); 0.818(1.0); |
| | | | 0.806(1.0); 0.799(2.0); 0.787(4.0); 0.781(5.5); 0.769(5.2); 0.763(4.3); 0.752(1.9); |
| | | | 0.730(0.4); 0.713(0.3); 0.694(0.4); 0.684(0.8); 0.673(0.8); 0.668(0.7); 0.632(1.9); |
| | | | 0.621(5.2); 0.615(4.7); 0.611(4.4); 0.606(4.3); 0.593(1.4); 0.146(0.8); |
| | | | 0.008(6.8); 0.000(210.4); −0.009(7.5); −0.150(0.9) |
| I-036 | 3.21 | 3.30 | Example I-036: ¹H-NMR(400.0 MHz, d₆-DMSO): δ = |
| | | | 11.099(4.0); 8.494(6.9); 7.989(5.8); 7.969(8.9); 7.902(10.0); 7.881(6.2); 7.741 |
| | | | (0.3); 7.594(5.4); 5.758(12.2); 3.506(0.4); 3.410(4.5); 3.017(16.0); 2.955(1.6); |
| | | | 2.942(2.0); 2.929(1.7); 2.863(1.3); 2.678(0.5); 2.673(0.6); 2.669(0.4); 2.509(67.8); |
| | | | 2.504(84.8); 2.500(60.7); 2.336(0.4); 2.331(0.5); 2.327(0.4); 2.039(1.6); 2.026(2.0); |
| | | | 2.014(1.5); 1.996(0.5); 1.233(0.4); 0.927(0.5); 0.908(0.4); 0.856(12.1); |
| | | | 0.838(6.7); 0.816(1.1); 0.700(0.6); 0.486(8.9); 0.473(10.9); 0.146(0.3); 0.008(3.7); |
| | | | 0.000(72.1); −0.008(3.1); −0.150(0.3) |
| I-037 | 3.98 | 4.05 | Example I-037: ¹H-NMR(400.0 MHz, d₆-DMSO): δ = |
| | | | 11.028(4.4); 8.568(6.0); 8.564(5.7); 8.313(1.3); 8.298(2.5); 8.283(1.2); 7.996(4.4); |
| | | | 7.987(7.4); 7.983(7.9); 7.976(6.9); 7.915(7.0); 7.894(4.2); 3.389(1.8); 3.373(4.4); |
| | | | 3.356(4.5); 3.330(36.7); 2.674(0.3); 2.509(42.9); 2.505(54.6); 2.500(39.1); |
| | | | 2.331(0.3); 2.083(0.5); 2.067(1.3); 2.053(1.9); 2.037(1.4); 2.022(0.5); 1.585(0.8); |
| | | | 1.568(2.5); 1.550(3.8); 1.531(2.9); 1.513(1.2); 1.410(0.6); 1.392(2.3); |
| | | | 1.372(3.5); 1.354(3.5); 1.336(2.0); 1.318(0.6); 0.946(8.0); 0.927(16.0); |
| | | | 0.909(7.0); 0.884(13.1); 0.872(6.9); 0.867(6.4); 0.008(3.4); |
| | | | 0.000(73.0); −0.008(3.1); −0.150(0.4) |
| I-038 | 3.25 | 3.34 | Example I-038: ¹H-NMR(400.0 MHz, d₆-DMSO): δ = |
| | | | 11.090(5.6); 8.613(1.7); 8.595(8.6); 8.591(7.8); 8.514(0.7); 8.007(5.3); 7.998(8.5); |
| | | | 7.994(8.9); 7.987(8.9); 7.969(0.6); 7.935(0.5); 7.919(8.8); 7.898(5.8); 7.592(0.4); |
| | | | 5.758(4.2); 4.182(5.2); 4.176(5.7); 4.168(5.7); 4.162(5.3); 3.331(103.7); |
| | | | 3.235(3.1); 3.229(6.4); 3.222(3.1); 3.016(0.5); 2.672(0.9); 2.507(113.0); |
| | | | 2.503(143.3); 2.499(105.4); 2.330(1.0); 2.082(0.6); 2.068(1.7); 2.052(2.4); |
| | | | 2.037(2.0); 2.021(0.7); 1.233(0.7); 0.930(0.4); 0.886(16.0); 0.873(9.0); 0.869(8.7); |
| | | | 0.472(0.4); 0.146(0.5); 0.008(4.4); 0.000(99.6); −0.008(5.0); −0.150(0.5) |
| I-039 | 4.31 | 4.36 | Example I-039: ¹H-NMR(400.0 MHz, d₆-DMSO): δ = |
| | | | 9.232(3.8); 9.211(3.8); 8.371(8.1); 8.317(0.6); 8.192(8.2); 8.131(7.0); 8.111(8.4); |
| | | | 7.973(0.5); 7.926(8.7); 7.906(7.3); 7.846(0.4); 7.820(8.4); 7.799(10.4); |
| | | | 7.648(10.1); 7.627(8.1); 5.316(0.6); 5.300(2.0); 5.281(3.0); 5.263(2.1); 5.245(0.6); |
| | | | 3.847(0.5); 3.696(0.5); 3.672(0.8); 3.655(0.7); 3.324(229.4); 2.995(0.3); 2.980 |
| | | | (0.4); 2.845(0.4); 2.840(0.4); 2.820(0.4); 2.671(3.7); 2.635(0.6); 2.615(0.8); |
| | | | 2.502(492.6); 2.370(0.4); 2.329(3.3); 2.055(1.5); 2.044(3.1); 2.035(3.9); 2.025(5.7); |
| | | | 2.014(4.1); 2.005(3.3); 1.994(1.7); 1.579(12.7); 1.561(12.7); 1.235(0.7); |
| | | | 1.061(10.1); 1.053(16.0); 1.043(11.9); 0.979(0.7); 0.953(1.7); 0.931(7.4); |
| | | | 0.923(7.0); 0.912(10.8); 0.903(6.9); 0.894(5.9); 0.871(1.6); 0.000(15.7) |
| I-040 | 3.94 | 4.01 | Example I-040: ¹H-NMR(400.0 MHz, d₆-DMSO): δ = |
| | | | 11.066(6.7); 8.773(3.8); 8.753(3.9); 8.588(8.5); 8.585(8.2); 7.992(5.7); 7.971(9.7); |
| | | | 7.965(10.8); 7.961(9.3); 7.909(9.6); 7.888(5.9); 7.847(9.5); 7.826(11.7); |
| | | | 7.641(10.7); 7.621(8.8); 5.758(11.0); 5.277(0.5); 5.259(2.0); 5.241(2.9); |
| | | | 5.223(2.0); 5.205(0.5); 3.329(101.2); 2.677(0.6); 2.673(0.7); 2.668(0.5); 2.526(2.5); |
| | | | 2.512(45.8); 2.508(89.1); 2.504(114.2); 2.499(81.3); 2.495(38.8); 2.335(0.6); |
| | | | 2.330(0.7); 2.326(0.5); 2.096(0.6); 2.081(1.9); 2.065(2.5); 2.049(1.9); |
| | | | 2.034(0.7); 1.568(13.1); 1.550(13.1); 1.233(0.8); 0.888(16.0); 0.876(8.4); 0.870(8.8); |
| | | | 0.146(0.7); 0.008(6.8); 0.000(164.0); −0.009(6.4); −0.150(0.8) |
| I-041 | 3.18 | 3.28 | Example I-041: ¹H-NMR(400.0 MHz, d₆-DMSO): δ = |
| | | | 10.996(5.2); 9.299(6.1); 8.839(1.8); 8.826(1.8); 8.580(6.7); 8.577(6.4); 8.370(0.7); |
| | | | 8.351(0.4); 8.320(0.3); 8.163(0.4); 8.146(0.4); 8.017(5.1); 7.997(8.2); 7.988(8.2); |
| | | | 7.985(7.2); 7.922(8.2); 7.901(5.6); 7.880(1.4); 7.863(0.9); 5.758(4.4); |
| | | | 4.304(0.5); 4.280(0.4); 4.160(0.4); 4.156(0.4); 4.142(0.4); 4.139(0.4); 4.127(0.4); |
| | | | 4.109(0.6); 4.092(0.6); 4.074(0.4); 3.982(0.3); 3.964(0.4); 3.947(0.3); 3.861 |
| | | | (0.3); 3.843(0.4); 3.631(1.9); 3.615(2.3): 3.599(2.3); 3.540(2.8); 3.521(2.8); |
| | | | 3.317(0.6); 3.304(0.5); 3.249(0.4); 3.225(0.3); 2.803(0.4); 2.780(0.6); 2.756(0.4); |
| | | | 2.672(1.2); 2.668(0.9); 2.507(137.4); 2.503(170.1); 2.499(126.6); 2.330(1.2); |
| | | | 2.216(0.4); 2.199(0.6); 2.180(0.6); 2.167(0.3); 2.159(0.3); 2.100(0.6); 2.085(1.7); |
| | | | 2.069(2.3); 2.054(2.0); 2.039(0.8); 1.637(2.4); 1.623(6.1); 1.616(6.5); 1.603(2.8); |
| | | | 1.584(0.5); 1.577(0.5); 1.563(0.5); 1.404(0.3); 1.363(3.2); 1.350(6.3); 1.343(6.5); |
| | | | 1.329(2.3); 1.278(0.3); 1.234(0.7); 1.222(0.8); 1.213(0.8); 1.205(1.3); |
| | | | 1.195(1.5); 1.187(1.0); 1.177(1.2); 1.170(0.4); 1.159(0.5); 1.107(0.4); 1.087(0.4); |
| | | | 1.064(0.5); 1.051(0.6); 1.041(0.7); 1.032(0.9); 1.024(1.0); 1.006(0.8); 0.990(0.8); |
| | | | 0.981(0.9); 0.972(0.8); 0.962(0.9); 0.949(0.6); 0.929(0.8); 0.920(1.0); |
| | | | 0.899(1.6); 0.878(16.0); 0.864(10.1); 0.000(63.8); −0.149(0.3) |
| I-042 | 3.26 | 3.36 | Example I-042: ¹H-NMR(400.0 MHz, d₆-DMSO): δ = |
| | | | 11.002(6.6); 9.070(1.9); 9.054(3.9); 9.039(1.9); 8.791(0.4); 8.773(6.0); 8.574(7.8); |
| | | | 8.571(7.8); 8.540(0.6); 8.044(2.7); 8.025(9.2); 8.011(15.4); 8.006(12.3); |
| | | | 7.986(11.3); 7.918(9.7); 7.897(6.3); 4.693(7.8); 4.678(7.8); 3.336(60.8); 2.673(0.7); |
| | | | 2.508(88.9); 2.504(111.2); 2.500(84.4); 2.331(0.7); 2.090(0.7); 2.077(2.8); |
| | | | 2.060(2.7); 2.044(1.9); 2.029(0.8); 1.232(1.2); 0.877(16.0); 0.866(9.6); 0.858 |
| | | | (8.2); 0.146(0.3); 0.000(69.3); −0.150(0.3) |
| I-043 | 3.58 | 3.67 | Example I-043: ¹H-NMR(400.0 MHz, d₆-DMSO): δ = |
| | | | 11.130(3.3); 11.096(1.7); 8.528(3.6); 8.525(3.8); 8.471(2.0); 8.011(3.0); 7.990 |
| | | | (4.3); 7.914(5.1); 7.896(7.3); 7.885(10.0); 7.877(6.0); 7.816(2.2); 7.796(2.5); |
| | | | 7.682(3.8); 7.679(4.0); 7.603(2.7); 7.595(4.7); 7.574(3.9); 7.486(2.2); 7.466(2.0); |
| | | | 4.796(6.9); 4.691(3.3); 3.331(55.6); 3.170(1.3); 2.957(16.0); 2.950(9.4); 2.673(0.5); |
| | | | 2.508(58.3); 2.504(74.1); 2.500(58.3); 2.331(0.5); 2.083(0.4); 2.066(1.0); |
| | | | 2.052(1.6); 2.035(1.4); 2.019(0.8); 1.231(0.9); 0.863(10.3); 0.846(7.0); 0.000(7.6) |
| I-044 | 3.76 | 3.90 | Example I-044: ¹H-NMR(400.0 MHz, d₆-DMSO): δ = |
| | | | 10.971(6.9); 8.957(1.8); 8.941(4.0); 8.925(2.0); 8.531(7.9); 8.527(8.6); 8.317(0.6); |
| | | | 7.982(8.7); 7.978(9.2); 7.918(9.8); 7.896(11.6); 7.839(9.7); 7.819(11.8); |
| | | | 7.556(10.6); 7.546(8.4); 7.536(10.2); 7.526(7.4); 5.757(3.4); 4.657(7.5); 4.641(7.7); |
| | | | 3.325(90.0); 2.676(0.9); 2.671(1.2); 2.667(0.9); 2.525(2.4); 2.507(145.6); |
| | | | 2.502(197.1); 2.498(152.3); 2.333(1.0); 2.329(1.4); 2.325(1.1); 2.072(0.7); |
| | | | 2.055(1.8); 2.048(1.4); 2.041(2.8); 2.025(1.9); 2.010(0.8); 1.989(0.9); 1.298(0.4); |
| | | | 1.259(0.5); 1.232(1.9); 1.193(0.3); 1.175(0.5); 0.869(16.0); 0.859(9.3); 0.850(8.0); |
| | | | 0.146(0.8); 0.008(6.0); 0.000(165.3); −0.008(9.3); −0.150(0.8) |
| I-045 | 3.99 | 4.14 | Example I-045: ¹H-NMR(400.0 MHz, d₆-DMSO): δ = |
| | | | 20.002(0.4); 10.481(9.1); 8.988(2.6); 8.973(5.2); 8.957(2.7); 8.639(8.6); 8.551 |
| | | | (0.3); 8.481(0.3); 8.317(1.1); 8.023(16.0); 8.009(13.5); 7.935(13.2); 7.914(9.0); |
| | | | 7.835(12.3); 7.815(13.9); 7.722(0.6); 7.675(0.6); 7.652(0.7); 7.592(0.6); 7.547(13.4); |
| | | | 7.527(11.7); 7.393(0.5); 7.340(0.4); 7.240(0.4); 7.176(0.5); 7.148(0.4); |
| | | | 6.936(0.3); 4.647(10.8); 4.632(10.8); 3.460(0.7); 3.440(2.6); 3.419(4.0); 3.398(2.9); |
| | | | 3.331(577.5); 2.996(1.1); 2.713(0.4); 2.671(4.6); 2.542(93.7); 2.502(740.3); |
| | | | 2.368(1.0); 2.329(5.1); 2.303(1.6); 2.279(4.0); 2.254(6.4); 2.230(5.6); 2.207(2.1); |
| | | | 2.169(2.6); 2.147(5.8); 2.126(5.4); 2.012(0.9); 1.988(2.2); 1.965(3.5); |
| | | | 1.941(3.7); 1.919(2.1); 1.896(0.8); 1.860(1.5); 1.836(3.0); 1.811(2.5); |
| | | | 1.508(0.4); 0.147(1.1); 0.002(158.9); 0.000(205.0); −0.150(1.0) |
| I-046 | 3.76 | 3.80 | Example I-046: ¹H-NMR(400.0 MHz, d₆-DMSO): δ = |
| | | | 11.012(5.8); 8.581(0.4); 8.562(7.3); 8.558(7.4); 8.464(0.3); 8.441(1.6); 8.426(3.4); |
| | | | 8.410(1.6); 8.316(0.5); 7.992(4.9); 7.970(16.0); 7.967(11.3); 7.917(8.6); |
| | | | 7.896(5.0); 7.797(8.6); 7.781(3.6); 7.777(10.2); 7.763(0.4); 7.501(9.1); 7.481(7.9); |
| | | | 7.401(0.4); 7.387(0.4); 7.382(0.4); 7.368(0.3); 3.665(1.7); 3.648(4.1); 3.631 |
| | | | (4.1); 3.614(1.9); 3.358(0.4); 3.327(191.6); 3.008(3.3); 2.990(6.0); 2.972(3.0); |
| | | | 2.676(0.8); 2.672(1.0); 2.667(0.8); 2.525(2.6); 2.512(56.2); 2.507(113.8); |
| | | | 2.503(150.2); 2.498(108.4); 2.494(51.8); 2.334(0.7); 2.330(1.0); 2.325(0.7); |
| | | | 2.321(0.3); 2.075(1.1); 2.060(1.6); 2.046(2.3); 2.030(1.8); 2.015(0.6); 0.880(15.6); |
| | | | 0.867(8.4); 0.863(7.9); 0.838(0.4); 0.008(0.5); 0.000(14.7); −0.009(0.5) |
| I-047 | 4.15 | 4.26 | Example I-047: ¹H-NMR(400.0 MHz, d₆-DMSO): δ = |
| | | | 11.229(9.8); 8.901(1.2); 8.676(1.6); 8.316(0.8); 8.105(8.5); 8.071(7.6); 8.051(10.7); |
| | | | 8.016(0.3); 7.956(13.0); 7.935(9.3); 7.898(0.8); 7.829(13.6); 7.808(16.0); |
| | | | 7.761(0.8); 7.740(1.0); 7.690(0.7); 7.670(0.8); 7.644(4.6); 7.629(5.4); 7.625(5.5); |
| | | | 7.584(2.9); 7.565(5.8); 7.540(11.8); 7.520(10.5); 7.481(3.7); 7.463(4.7); |
| | | | 7.446(2.3); 7.420(0.8); 7.381(0.6); 7.359(0.5); 7.328(0.4); 7.238(0.3); 7.200(0.4); |
| | | | 7.180(0.4); 7.115(0.3); 4.653(6.3); 4.639(6.4); 3.326(491.1); 2.996(2.9); 2.712(0.9); |
| | | | 2.675(1.8); 2.671(2.4); 2.667(1.8); 2.542(203.2); 2.507(270.8); 2.502(350.9); |
| | | | 2.498(266.0); 2.449(1.8); 2.368(1.0); 2.333(1.9); 2.329(2.5); 2.325(1.9); |
| | | | 2.291(0.3); 0.146(1.0); 0.000(213.2); −0.008(9.9); −0.150(1.0) |
| I-048 | 3.47 | 4.59 | Example I-048: ¹H-NMR(400.0 MHz, d₆-DMSO): δ = |
| | | | 10.629(4.1); 8.973(1.8); 8.604(4.9); 8.024(8.2); 8.020(10.7); 7.999(7.2); 7.932 |
| | | | (7.5); 7.911(4.7); 7.838(7.1); 7.817(8.4); 7.551(7.4); 7.531(6.5); 4.653(5.7); |
| | | | 4.637(5.7); 3.449(0.4); 3.439(0.5); 3.351(352.3); 3.347(289.4); 3.342(291.4); |
| | | | 3.340(265.9); 2.677(1.0); 2.673(1.4); 2.668(1.1); 2.526(4.0); 2.508(158.0); |
| | | | 2.503(209.9); 2.499(161.8); 2.475(7.5); 2.456(6.6); 2.437(2.2); 2.335(1.0); |
| | | | 2.330(1.3); 2.326(1.0); 1.328(0.3); 1.233(0.9); 1.121(7.6); 1.102(16.0); 1.083(7.3); |
| | | | 0.146(0.6); 0.008(5.0); 0.000(121.6); −0.008(6.4); −0.150(0.6) |
| I-049 | 4.10 | 4.21 | Example I-049: ¹H-NMR(400.0 MHz, d₆-DMSO): δ = |
| | | | 11.031(5.5); 8.569(6.9); 8.566(7.4); 8.316(1.3); 8.291(1.5); 8.276(3.1); 8.261(1.6); |
| | | | 7.993(11.0); 7.989(9.5); 7.976(8.5); 7.914(8.5); 7.893(5.4); 3.424(4.2); |
| | | | 3.407(6.9); 3.390(4.5); 3.322(172.6); 3.292(1.4); 2.675(2.0); 2.671(2.7); 2.667(2.0); |
| | | | 2.594(0.9); 2.575(2.0); 2.557(3.0); 2.538(3.3); 2.510(150.3); 2.506(300.7); |
| | | | 2.502(402.2); 2.497(307.3); 2.333(2.0); 2.329(2.7); 2.324(2.1); 2.079(0.7); |
| | | | 2.072(1.1); 2.063(2.3); 2.048(3.7); 2.033(4.5); 2.022(2.4); 2.015(2.9); 1.998(1.1); |
| | | | 1.993(1.3); 1.904(0.8); 1.900(0.8); 1.882(2.4); 1.865(4.3); 1.845(4.0); 1.829(1.7); |
| | | | 1.781(1.4); 1.764(2.6); 1.738(3.2); 1.715(2.4); 1.695(0.6); 1.234(4.4); 0.937 |
| | | | (0.3); 0.883(16.0); 0.869(9.4); 0.866(9.1); 0.837(1.3); 0.146(1.9); 0.008(17.4); |
| | | | 0.000(407.7); −0.008(23.1); −0.031(2.4); −0.150(1.9) |
| I-050 | 3.83 | 3.87 | Example I-050: ¹H-NMR(400.0 MHz, d₆-DMSO): δ = |
| | | | 10.954(5.8); 10.021(0.8); 10.006(1.7); 9.990(0.9); 8.945(1.6); 8.930(3.5); |
| | | | 8.913(1.7); 8.519(7.0); 8.516(7.3); 8.482(7.4); 8.468(0.5); 8.316(0.5); 7.960(7.7); |
| | | | 7.957(8.0); 7.947(2.8); 7.925(11.5); 7.903(10.4); 7.877(0.5); 7.836(13.3); 7.816 |
| | | | (16.0); 7.793(1.4); 7.781(1.1); 7.745(3.4); 7.723(2.8); 7.711(0.9); 7.689(0.9); |
| | | | 7.664(0.7); 7.624(0.7); 7.604(0.8); 7.583(10.2); 7.562(9.8); 7.551(14.0); |
| | | | 7.530(12.0); 7.501(0.9); 7.488(0.7); 7.393(0.6); 7.372(0.5); 7.315(0.4); 7.279(0.3); |
| | | | 4.733(3.0); 4.717(3.0); 4.652(6.9); 4.636(6.9); 4.574(0.4); 3.962(1.3); 3.913(0.5); |
| | | | 3.327(213.3); 2.676(1.2); 2.672(1.6); 2.667(1.2); 2.507(185.5); 2.503(243.2); |
| | | | 2.498(180.3); 2.334(1.3); 2.329(1.7); 2.325(1.3); 2.075(0.7); 2.065(0.7); 2.047(1.5); |
| | | | 2.041(1.3); 2.034(2.5); 2.021(1.6); 2.003(0.8); 1.505(0.4); 1.233(0.4); |
| | | | 0.865(12.2); 0.857(8.6); 0.845(6.5); 0.823(0.8); 0.146(1.1); 0.008(9.4); |
| | | | 0.000(226.3); −0.150(1.1) |
| I-051 | 3.37 | 3.49 | Example I-051: ¹H-NMR(400.0 MHz, d₆-DMSO): δ = |
| | | | 9.799(2.2); 9.789(4.4); 9.779(2.0); 8.763(7.0); 8.691(0.6); 8.654(16.0); 8.594(0.4); |
| | | | 8.520(0.6); 8.504(7.8); 8.490(8.1); 8.322(0.6); 8.034(3.9); 8.021(8.6); 8.004(5.4); |
| | | | 8.001(5.0); 7.991(2.7); 7.988(2.5); 7.970(8.7); 7.956(8.0); 5.761(15.7); |
| | | | 5.336(0.4); 4.658(8.2); 4.648(8.1); 3.991(1.0); 3.734(0.4); 3.703(0.4); 3.682(1.3); |
| | | | 3.659(0.5); 3.639(0.4); 3.627(0.4); 3.610(0.5); 3.485(1.1); 3.436(3.9); 3.406(3.0); |
| | | | 3.350(0.6); 3.343(0.4); 2.616(1.2); 2.522(3.3); 2.507(148.1); 2.504(199.6); |
| | | | 2.501(148.7); 2.388(1.2); 2.064(1.5); 2.057(3.3); 2.051(3.9); 2.044(6.5); |
| | | | 2.036(4.3); 2.031(3.6); 2.023(1.9); 1.245(0.4); 1.233(0.8); 1.074(3.4); 1.067(9.9); |
| | | | 1.061(14.3); 1.055(11.4); 1.049(4.8); 1.031(1.0); 1.025(0.8); 1.004(0.4); |
| | | | 0.990(0.7); 0.964(4.5); 0.959(10.1); 0.953(10.4); 0.946(11.5); 0.940(8.9); |
| | | | 0.934(3.5); 0.910(0.8); 0.902(0.8); 0.897(0.7); 0.866(0.9); 0.822(0.6); 0.811(0.6); 0.768 |
| | | | (0.4); 0.751(0.4); 0.096(0.8); 0.005(7.9); 0.000(199.3); −0.006(11.1); −0.100(1.0) |
| I-052 | 4.01 | 4.19 | Example I-052: ¹H-NMR(400.0 MHz, d₆-DMSO): δ = |
| | | | 11.004(6.1); 8.969(1.6); 8.953(3.6); 8.938(1.9); 8.562(6.8); 8.558(7.7); 8.536(0.8); |
| | | | 8.317(0.9); 8.086(0.3); 8.008(7.3); 8.005(8.1); 7.969(0.3); 7.941(4.9); 7.920(13.0); |
| | | | 7.892(11.6); 7.871(5.3); 7.840(9.2); 7.819(11.3); 7.792(1.2); 7.739(0.8); |
| | | | 7.712(0.8); 7.691(0.8); 7.654(1.0); 7.620(0.9); 7.598(0.7); 7.557(9.6); 7.537(8.9); |
| | | | 7.402(0.7); 7.385(0.6); 7.243(0.4); 7.221(0.4); 5.757(0.4); 4.725(0.4); |
| | | | 4.658(6.7); 4.642(6.9); 3.328(233.6); 2.671(2.1); 2.667(1.6); 2.507(237.9); |
| | | | 2.502(320.9); 2.498(259.0); 2.329(2.4); 2.325(2.0); 2.179(0.4); 2.075(0.7); |
| | | | 2.058(1.8); 2.044(2.6); 2.028(2.1); 2.013(0.9); 1.298(0.4); 1.234(3.7); 1.192(0.5); |
| | | | 1.174(0.6); 1.156(0.4); 1.036(0.6); 0.872(16.0); 0.860(9.8); 0.854(9.5); 0.146(0.4); |
| | | | 0.000(88.7); −0.150(0.4) |
| I-053 | 3.61 | 3.73 | Example I-053: ¹H-NMR(400.0 MHz, d₆-DMSO): δ = |
| | | | 10.964(3.4); 10.030(0.4); 10.015(0.7); 9.999(0.4); 8.952(0.9); 8.936(2.0); 8.920(1.0); |
| | | | 8.525(4.2); 8.521(4.4); 8.491(3.4); 8.317(0.3); 7.969(4.6); 7.965(4.8); 7.908(1.0); |
| | | | 7.887(1.3); 7.838(7.1); 7.818(8.6); 7.791(0.5); 7.776(1.6); 7.761(1.3); |
| | | | 7.754(1.9); 7.739(15.9); 7.738(16.0); 7.722(0.8); 7.714(1.0); 7.649(0.3); |
| | | | 7.642(0.3); 7.627(0.3); 7.597(0.3); 7.589(0.3); 7.553(7.0); 7.532(6.3); 7.511(0.5); |
| | | | 7.495(0.4); 7.483(0.4); 7.404(0.4); 7.218(0.4); 7.213(0.4); 5.758(11.2); 4.735(1.2); |
| | | | 4.720(1.3); 4.654(3.8); 4.638(3.8); 3.329(74.1); 2.677(0.5); 2.672(0.7); |
| | | | 2.668(0.5); 2.525(1.5); 2.512(38.8); 2.507(80.5); 2.503(109.1); 2.498(83.3); |
| | | | 2.494(43.5); 2.334(0.6); 2.330(0.8); 2.325(0.6); 2.068(0.4); 2.050(0.9); 2.044(0.7); |
| | | | 2.037(1.4); 2.024(0.9); 2.020(0.9); 2.007(0.5); 1.232(1.0); 0.901(0.4); 0.893(0.5); |
| | | | 0.867(7.2); 0.858(4.8); 0.847(3.9); 0.649(0.5); 0.008(1.1); 0.000(34.3); −0.008(1.6) |
| I-054 | 4.59 | 4.68 | Example I-054: ¹H-NMR(400.0 MHz, d₆-DMSO): δ = |
| | | | 11.033(4.8); 10.708(0.3); 9.216(0.5); 8.913(1.4); 8.899(2.9); 8.883(1.4); 8.761 |
| | | | (0.5); 8.745(0.7); 8.734(0.4); 8.689(0.7); 8.674(0.3); 8.621(0.4); 8.607(0.5); |
| | | | 8.588(6.0); 8.585(5.9); 8.535(0.4); 8.499(0.5); 8.438(0.4); 8.316(6.0); 8.238(0.5); |
| | | | 8.217(0.4); 8.188(0.5); 8.166(0.5); 8.125(0.7); 8.112(0.9); 8.066(0.9); 8.021(7.9); |
| | | | 8.018(7.8); 8.006(5.3); 7.985(8.1); 7.939(1.8); 7.933(2.4); 7.919(8.3); 7.898(5.8); |
| | | | 7.865(1.7); 7.856(1.7); 7.847(2.2); 7.831(1.9); 7.798(1.5); 7.781(1.9); |
| | | | 7.760(1.9); 7.719(4.6); 7.693(4.8); 7.659(3.1); 7.641(4.6); 7.622(5.8); 7.613(6.0); |
| | | | 7.589(3.2); 7.582(2.6); 7.568(2.2); 7.533(2.0); 7.517(1.5); 7.476(1.5); 7.456 |
| | | | (1.4); 7.440(1.1); 7.429(1.1); 7.420(1.2); 7.413(1.3); 7.385(1.3); 7.380(1.3); |
| | | | 7.367(1.3); 7.346(1.2); 7.335(1.5); 7.313(1.1); 7.307(1.2); 7.300(1.0); 7.283(1.1); |
| | | | 7.259(1.0); 7.244(1.1); 7.212(1.1); 7.203(1.0); 7.182(0.9); 7.173(1.0); 7.151(0.7); |
| | | | 7.139(0.5); 7.124(0.5); 7.112(0.6); 7.102(0.7); 7.087(0.6); 7.081(0.7); 7.064(0.6); |
| | | | 7.039(0.6); 7.010(0.4); 7.001(0.4); 6.959(0.7); 6.809(0.4); 6.496(0.6); |
| | | | 5.264(0.3); 4.926(0.4); 4.773(0.3); 4.741(0.5); 4.694(4.6); 4.679(4.7); 4.640(0.5); |
| | | | 4.620(0.5); 4.595(1.0); 4.583(1.0); 4.557(0.3); 4.528(0.4); 4.514(0.4); 4.494 |
| | | | (0.5); 4.474(0.5); 4.435(0.5); 4.319(0.3); 4.310(0.3); 4.306(0.3); 4.220(0.5); |
| | | | 4.182(0.4); 4.157(0.3); 4.130(0.4); 4.109(0.3); 4.087(0.3); 4.066(0.4); 4.059(0.3); |
| | | | 4.033(0.4); 4.005(0.4); 3.986(0.4); 3.967(0.8); 3.954(0.5); 3.927(0.5); 3.916(0.5); |
| | | | 3.895(0.4); 3.888(0.4); 3.884(0.4); 3.865(0.5); 3.860(0.5); 3.848(0.5); 3.789(0.6); |
| | | | 3.772(0.6); 3.756(0.6); 3.706(0.8); 3.693(0.8); 3.678(0.8); 3.654(0.9); |
| | | | 3.639(0.9); 3.614(1.0); 3.606(0.9); 3.572(1.0); 3.558(1.0); 3.534(1.1); 3.527(1.0); |
| | | | 3.508(1.3); 3.487(1.1); 3.459(3.3); 3.327(2666.6); 3.052(0.4); 3.044(0.4); |
| | | | 3.030(0.4); 2.967(0.6); 2.948(0.5); 2.943(0.5); 2.892(0.6); 2.871(0.6); 2.855(0.7); |
| | | | 2.825(0.8); 2.802(0.8); 2.764(0.9); 2.750(1.0); 2.728(1.0); 2.719(1.0); 2.676(9.8); |
| | | | 2.671(13.3); 2.667(10.2); 2.662(5.4); 2.524(35.8); 2.511(684.9); 2.507(1394.3); |
| | | | 2.502(1845.5); 2.498(1345.0); 2.493(653.3); 2.333(8.0); 2.329(11.4); |
| | | | 2.324(8.2); 2.281(0.4); 2.201(0.6); 2.188(0.4); 2.164(1.2); 2.078(0.5); 2.067(1.3); |
| | | | 2.049(2.0); 2.033(1.4); 2.017(0.8); 1.996(0.5); 1.966(0.4); 1.835(0.5); 1.661 |
| | | | (0.4); 1.650(0.4); 1.634(0.7); 1.620(0.3); 1.617(0.4); 1.608(0.3); 1.602(0.4); |
| | | | 1.574(0.5); 1.550(0.8); 1.535(0.6); 1.504(1.0); 1.493(0.9); 1.481(0.6); 1.457(0.5); |
| | | | 1.427(0.4); 1.423(0.4); 1.408(0.3); 1.351(0.8); 1.297(1.3); 1.273(4.9); 1.267(4.5); |
| | | | 1.258(3.9); 1.235(16.0); 1.188(1.5); 1.175(1.3); 1.166(1.3); 1.148(1.3); |
| | | | 1.145(1.3); 1.107(1.5); 1.103(1.5); 1.085(1.6); 1.082(1.6); 1.043(2.3); 1.031(2.3); |
| | | | 1.021(2.3); 1.013(2.1); 0.976(2.0); 0.967(2.3); 0.927(3.2); 0.907(3.1); |
| | | | 0.877(13.8); 0.866(10.2); 0.856(10.0); 0.836(4.9); 0.752(1.5); 0.741(1.5); 0.725(1.1); |
| | | | 0.716(0.9); 0.680(0.7); 0.671(0.9); 0.646(0.7); 0.630(0.7); 0.604(0.6); 0.580(0.5); |
| | | | 0.564(0.5); 0.146(0.6); 0.065(0.4); 0.008(5.7); 0.000(175.4); −0.009(6.5); −0.150(0.7) |
| I-055 | 3.82 | 3.92 | Example I-055: ¹H-NMR(400.0 MHz, d₆-DMSO): δ = 0.20(m, 2H), |
| | | | 0.50(m, 2H), 1.10(m, 1H), 2.35(d, 2H), 4.64(d, 2H), 7.55(d, 2H), 7.82(d, |
| | | | 2H), 7.93(d, 2H), 8.03(s, d, 3H), 8.63(s, 1H), 8.96(t, 1H), 10.58(s, 1H) |
| I-056 | 4.44 | 4.57 | Example I-056: ¹H-NMR(400.0 MHz, d₆-DMSO): δ = |
| | | | 11.001(7.0); 8.918(1.9); 8.902(4.1); 8.886(2.0); 8.578(8.3); 8.574(8.6); 8.317(0.6); |
| | | | 8.021(9.2); 8.018(9.6); 8.007(6.5); 7.987(9.9); 7.919(10.2); 7.898(6.7); |
| | | | 7.597(3.6); 7.577(7.0); 7.556(4.1); 7.421(3.4); 7.416(3.8); 7.395(3.5); 7.390(3.7); |
| | | | 7.248(3.7); 7.245(3.7); 7.227(3.3); 7.224(3.3); 5.757(2.7); 4.580(8.2); 4.565(8.3); |
| | | | 3.380(19.8); 2.676(1.0); 2.672(1.4); 2.668(1.1); 2.507(159.5); 2.503(212.5); |
| | | | 2.499(164.9); 2.334(1.0); 2.330(1.4); 2.325(1.1); 2.077(0.6); 2.060(1.7); |
| | | | 2.053(1.4); 2.046(2.8); 2.029(1.9); 2.015(0.8); 1.234(0.9); 0.876(16.0); 0.866(9.6); |
| | | | 0.856(8.1); 0.146(0.7); 0.008(5.2); 0.000(137.2); −0.007(9.3); −0.150(0.7) |
| I-057 | 4.77 | 4.91 | Example I-057: ¹H-NMR(400.0 MHz, d₆-DMSO): δ = |
| | | | 11.066(6.2); 8.746(3.6); 8.726(3.7); 8.590(7.7); 8.586(8.2); 8.316(0.8); 7.992(5.1); |
| | | | 7.970(16.0); 7.966(11.8); 7.908(8.8); 7.887(5.7); 7.872(0.7); 7.823(0.5); |
| | | | 7.814(0.5); 7.800(0.7); 7.793(0.6); 7.741(5.4); 7.720(9.3); 7.663(8.4); 7.643(5.4); |
| | | | 7.615(0.4); 7.513(0.7); 7.493(0.6); 7.459(0.5); 7.455(0.5); 7.441(0.4); 7.438(0.4); |
| | | | 7.250(0.3); 6.925(0.4); 6.922(0.4); 5.291(0.4); 5.274(1.6); 5.255(2.5); |
| | | | 5.237(1.7); 5.219(0.5); 4.056(0.5); 4.038(1.4); 4.020(1.4); 4.003(0.5); 3.324(130.1); |
| | | | 2.687(0.4); 2.676(1.3); 2.672(1.7); 2.667(1.2); 2.525(3.1); 2.520(5.2); 2.511 |
| | | | (87.3); 2.507(183.3); 2.503(249.7); 2.498(191.1); 2.494(103.3); 2.334(1.3); |
| | | | 2.329(1.8); 2.325(1.4); 2.088(0.7); 2.073(1.7); 2.057(2.4); 2.041(1.9); 2.026(0.8); |
| | | | 1.989(6.3); 1.585(12.1); 1.567(12.3); 1.539(0.7); 1.525(0.4); 1.352(0.4); 1.298(1.0); |
| | | | 1.279(0.5); 1.259(1.5); 1.233(4.0); 1.193(2.1); 1.175(3.8); 1.158(2.3); 0.887(14.0); |
| | | | 0.875(7.9); 0.868(8.4); 0.836(0.8); 0.146(1.0); 0.067(0.3); 0.008(8.5); |
| | | | 0.000(247.5); −0.008(16.2); −0.062(1.9); −0.150(1.1) |
| I-058 | 4.79 | 4.94 | Example I-058: ¹H-NMR(400.0 MHz, d₆-DMSO): δ = |
| | | | 11.010(6.3); 8.706(0.3); 8.566(7.5); 8.562(8.2); 8.410(1.6); 8.395(3.5); 8.380(1.8); |
| | | | 8.317(0.6); 7.996(5.3); 7.982(10.6); 7.978(14.8); 7.916(9.2); 7.895(5.6); |
| | | | 7.656(6.6); 7.635(8.5); 7.497(7.6); 7.477(6.3); 3.418(6.8); 3.401(8.5); 3.385(6.9); |
| | | | 3.368(3.2); 2.784(3.1); 2.765(5.4); 2.746(3.5); 2.676(0.8); 2.672(1.1); 2.667(0.9); |
| | | | 2.525(2.2); 2.512(60.3); 2.507(125.9); 2.503(171.8); 2.498(131.3); 2.494 |
| | | | (69.6); 2.334(0.9); 2.330(1.2); 2.325(0.9); 2.085(0.6); 2.069(1.7); 2.061(1.2); |
| | | | 2.055(2.5); 2.039(1.9); 2.024(0.7); 1.948(1.1); 1.930(3.1); 1.911(4.3); 1.893(3.1); |
| | | | 1.875(1.1); 1.233(0.8); 0.883(16.0); 0.871(8.5); 0.865(8.0); 0.812(0.3); 0.146 |
| | | | (1.1); 0.008(9.5); 0.000(262.3); −0.009(15.1); −0.150(1.2) |
| I-059 | 4.93 | 5.09 | Example I-059: ¹H-NMR(400.0 MHz, d₆-DMSO): δ = |
| | | | 11.023(7.1); 8.891(2.0); 8.876(4.3); 8.860(2.0); 8.594(8.5); 8.591(8.8); 8.317(0.8); |
| | | | 8.026(9.5); 8.022(9.8); 8.010(6.5); 7.990(10.3); 7.920(10.6); 7.899(6.9); |
| | | | 7.880(7.1); 7.863(7.2); 7.471(6.3); 7.446(6.3); 5.757(3.4); 4.615(8.5); 4.600(8.5); |
| | | | 3.346(60.7); 2.892(0.6); 2.732(0.6); 2.676(1.6); 2.672(2.2); 2.667(1.7); 2.542 |
| | | | (2.7); 2.525(6.1); 2.507(259.8); 2.503(341.2); 2.498(253.8); 2.334(1.6); 2.329(2.2); |
| | | | 2.325(1.6); 2.095(0.7); 2.078(1.7); 2.071(1.4); 2.064(2.8); 2.047(1.9); 2.033(0.8); |
| | | | 1.233(3.0); 0.881(16.0); 0.871(9.7); 0.861(8.0); 0.837(0.9); 0.812(0.5); 0.146(3.4); |
| | | | 0.067(1.1); 0.008(29.3); 0.000(658.7); −0.008(32.0); −0.041(0.4); −0.150(3.3) |
| I-060 | 4.82 | 4.89 | Example I-060: ¹H-NMR(400.0 MHz, d₆-DMSO): δ = |
| | | | 20.010(0.5); 11.069(4.7); 8.728(3.4); 8.588(5.6); 8.377(0.5); 8.318(0.4); 8.191 |
| | | | (0.6); 8.112(0.9); 7.970(12.8); 7.908(9.0); 7.721(9.5); 7.662(9.8); 7.400(1.1); |
| | | | 5.255(3.0); 3.683(0.4); 3.570(0.4); 3.325(45.9); 2.670(3.2); 2.503(287.9); 2.331 |
| | | | (4.2); 2.056(3.8); 1.580(11.9); 1.233(2.3); 1.055(2.2); 0.883(16.0); 0.146(2.2); |
| | | | 0.000(170.9); −0.150(2.2) |
| I-061 | 4.89 | 5.01 | Example I-061: ¹H-NMR(400.0 MHz, d₆-DMSO): δ = |
| | | | 11.031(6.4); 8.730(3.7); 8.711(3.9); 8.542(8.1); 8.538(8.6); 8.335(0.3); 8.330(0.4); |
| | | | 8.317(0.7); 8.130(0.3); 8.126(0.3); 8.053(0.4); 8.031(0.4); 7.928(9.0); |
| | | | 7.924(9.4); 7.909(1.1); 7.902(10.1); 7.897(3.6); 7.885(3.4); 7.880(11.9); |
| | | | 7.873(1.5); 7.740(5.9); 7.719(10.1); 7.688(0.8); 7.660(9.3); 7.640(5.8); 7.571(0.3); |
| | | | 7.536(7.3); 7.515(6.4); 5.756(0.7); 5.286(0.5); 5.268(1.8); 5.250(2.7); 5.232(1.8); |
| | | | 5.215(0.5); 3.660(0.3); 3.326(107.3); 2.676(1.0); 2.672(1.4); 2.667(1.1); 2.525(3.1); |
| | | | 2.512(80.5); 2.507(165.8); 2.503(221.3); 2.498(161.5); 2.494(79.0); 2.334(1.1); |
| | | | 2.330(1.5); 2.325(1.1); 2.082(0.7); 2.067(1.8); 2.051(2.5); 2.036(2.1); |
| | | | 2.020(0.8); 1.581(13.4); 1.564(13.3); 1.233(1.8); 1.058(0.6); 1.049(0.8); |
| | | | 1.039(0.8); 0.920(0.6); 0.881(16.0); 0.868(8.4); 0.863(8.9); 0.146(2.1); 0.008(17.4); |
| | | | 0.000(442.3); −0.009(17.0); −0.150(2.1) |
| I-062 | 4.53 | 4.67 | Example I-062: ¹H-NMR(400.0 MHz, d₆-DMSO): δ = |
| | | | 10.962(6.5); 8.900(1.8); 8.885(3.8); 8.869(1.8); 8.528(8.1); 8.524(8.5); 8.381(0.6); |
| | | | 8.377(0.6); 8.317(1.1); 8.163(0.8); 8.159(0.7); 8.072(0.8); 8.067(0.3); 8.050(0.9); |
| | | | 7.979(9.2); 7.975(9.3); 7.924(1.1); 7.917(10.3); 7.912(3.5); 7.900(3.5); |
| | | | 7.895(11.9); 7.887(1.3); 7.595(3.8); 7.575(7.2); 7.554(5.6); 7.545(7.4); 7.525 |
| | | | (6.4); 7.417(3.5); 7.413(3.7); 7.391(3.5); 7.387(3.7); 7.375(0.7); 7.358(0.4); |
| | | | 7.344(0.7); 7.298(0.5); 7.293(0.5); 7.245(3.5); 7.241(3.5); 7.224(3.4); 7.221(3.4); |
| | | | 7.198(0.5); 7.144(0.3); 7.139(0.3); 5.756(1.5); 4.575(7.5); 4.559(7.5); 4.520(0.6); |
| | | | 4.505(0.5); 3.652(0.8); 3.327(205.2); 2.676(1.4); 2.672(2.0); 2.667(1.5); |
| | | | 2.663(0.8); 2.569(0.4); 2.525(4.7); 2.512(107.0); 2.507(220.2); 2.503(293.9); |
| | | | 2.498(213.2); 2.494(102.8); 2.390(0.4); 2.334(1.4); 2.329(1.9); 2.325(1.4); |
| | | | 2.321(0.7); 2.070(0.6); 2.053(1.6); 2.046(1.2); 2.039(2.7); 2.022(2.0); 2.008(0.9); |
| | | | 1.283(0.4); 1.258(0.4); 1.234(1.8); 1.052(0.5); 1.042(0.9); 1.033(1.2); 1.023(1.1); |
| | | | 1.015(0.5); 1.003(0.4); 0.985(0.4); 0.975(0.3); 0.924(0.6); 0.916(1.0); 0.905(1.0); |
| | | | 0.896(1.3); 0.870(16.0); 0.859(8.6); 0.850(7.5); 0.827(0.6); 0.146(1.5); |
| | | | 0.008(11.7); 0.000(342.0); −0.008(12.2); −0.150(1.5) |
| I-063 | 5.07 | 5.20 | Example I-063: ¹H-NMR(400.0 MHz, d₆-DMSO): δ = |
| | | | 10.985(5.8); 8.874(2.1); 8.860(3.6); 8.846(2.1); 8.543(6.9); 7.981(7.3); 7.920(7.2); |
| | | | 7.899(8.1); 7.881(4.7); 7.864(4.3); 7.549(7.1); 7.529(6.6); 7.467(4.1); 7.443(4.1); |
| | | | 7.392(0.4); 7.365(0.3); 7.348(0.4); 7.319(0.4); 5.758(1.7); 4.610(7.3); |
| | | | 4.595(7.4); 3.996(0.3); 3.334(80.3); 2.671(2.0); 2.502(334.0); 2.329(2.6); |
| | | | 2.180(0.5); 2.129(0.3); 2.070(2.2); 2.057(2.9); 2.043(2.4); 1.989(0.5); 1.553(0.4); |
| | | | 1.510(0.4); 1.476(0.4); 1.458(0.3); 1.396(0.4); 1.350(0.5); 1.298(1.0); 1.234(5.8); |
| | | | 1.067(0.5); 1.042(0.5); 1.028(0.5); 0.875(16.0); 0.709(0.4); 0.695(0.4); −0.001(18.2) |
| I-064 | 4.53 | 4.68 | Example I-064: ¹H-NMR(400.0 MHz, d₆-DMSO): δ = |
| | | | 10.999(7.4); 9.034(2.1); 9.019(4.4); 9.003(2.1); 8.582(9.3); 8.578(9.5); 8.309(1.4); |
| | | | 8.025(10.2); 8.021(10.4); 8.007(6.8); 7.986(10.8); 7.920(11.2); 7.899(7.5); |
| | | | 7.882(0.9); 7.797(2.9); 7.778(5.7); 7.758(3.2); 7.734(0.4); 7.697(0.4); 7.649(0.3); |
| | | | 7.606(0.4); 7.573(0.3); 7.496(4.6); 7.466(4.3); 7.437(0.5); 7.406(4.7); 7.386(4.1); |
| | | | 7.356(0.3); 7.342(0.4); 6.982(0.5); 6.313(0.4); 4.668(7.5); 4.652(7.5); |
| | | | 4.596(0.5); 4.581(0.5); 4.124(0.8); 4.110(1.8); 4.097(1.9); 4.084(0.7); 3.794(0.4); |
| | | | 3.781(0.5); 3.726(0.4); 3.700(0.5); 3.678(0.5); 3.629(0.6); 3.574(0.8); 3.347 |
| | | | (4255.0); 3.258(0.3); 3.177(8.7); 3.164(8.5); 2.677(3.4); 2.673(4.5); 2.668(3.5); |
| | | | 2.642(0.5); 2.634(0.4); 2.611(0.5); 2.598(0.5); 2.508(543.7); 2.504(748.1); |
| | | | 2.499(584.6); 2.335(3.1); 2.330(4.3); 2.326(3.3); 2.072(8.9); 2.049(3.0); 2.034(1.9); |
| | | | 2.018(0.8); 1.356(0.4); 1.234(1.4); 0.881(16.0); 0.872(9.9); 0.861(8.0); 0.838(0.6); |
| | | | 0.146(4.3); 0.008(36.1); 0.000(938.5); −0.008(56.6); −0.034(0.5); −0.150(4.2) |
| I-065 | 4.74 | 4.80 | Example I-065: ¹H-NMR(400.0 MHz, d₆-DMSO): δ = 0.87(m, 4H), |
| | | | 2.05(m, 1H), 4.66(d, 2H), 7.40(d, 1H), 7.47(d, 1H), 7.55(d, 2H), 7.78(t, |
| | | | 1H), 7.90(d, 2H), 7.98(s, 1H), 8.53(s, 1H), 9.00(t, 1H), 10.97(s, 1H) |
| I-066 | 4.53 | 4.63 | Example I-066: ¹H-NMR(400.0 MHz, d₆-DMSO): δ = |
| | | | 11.030(1.1); 8.775(0.6); 8.585(1.4); 8.581(1.5); 8.016(1.6); 8.013(1.7); 8.003(1.0); |
| | | | 7.982(1.6); 7.917(1.6); 7.896(1.0); 7.474(1.0); 7.470(0.8); 7.451(1.2); 7.445(0.7); |
| | | | 7.432(0.6); 7.316(0.7); 7.311(0.6); 7.295(0.5); 7.290(0.5); 4.603(1.2); |
| | | | 4.588(1.2); 3.327(14.9); 2.525(0.5); 2.512(11.0); 2.508(22.2); 2.503(29.2); |
| | | | 2.498(21.3); 2.494(10.4); 2.041(0.5); 1.397(16.0); 1.232(0.3); 0.875(2.6); |
| | | | 0.865(1.4); 0.855(1.2); 0.008(0.8); 0.000(21.3); −0.008(0.8) |
| I-067 | 4.60 | 4.78 | Example I-067: ¹H-NMR(400.0 MHz, d₆-DMSO): δ = |
| | | | 10.993(2.0); 8.775(0.5); 8.760(1.1); 8.745(0.5); 8.535(2.8); 8.531(2.9); 8.488(1.3); |
| | | | 8.317(0.5); 8.081(0.4); 8.059(0.4); 7.973(3.1); 7.969(3.2); 7.921(0.4); 7.913(3.4); |
| | | | 7.908(1.1); 7.896(1.1); 7.891(4.0); 7.884(0.5); 7.545(2.4); 7.525(2.1); |
| | | | 7.474(1.4); 7.469(2.3); 7.449(3.3); 7.444(1.9); 7.427(1.3); 7.403(0.5); 7.383(0.4); |
| | | | 7.314(1.3); 7.310(1.4); 7.294(1.1); 7.289(1.1); 7.279(0.4); 4.657(0.4); 4.643 |
| | | | (0.4); 4.597(2.2); 4.582(2.2); 3.329(127.8); 2.676(1.0); 2.671(1.4); 2.667(1.0); |
| | | | 2.662(0.5); 2.525(3.3); 2.520(5.1); 2.511(80.0); 2.507(165.9); 2.502(221.1); |
| | | | 2.498(160.7); 2.493(77.6); 2.338(0.6); 2.333(1.1); 2.329(1.5); 2.324(1.1); |
| | | | 2.320(0.6); 2.298(0.3); 2.048(0.6); 2.034(0.9); 2.018(0.6); 1.989(0.3); 1.398(16.0); |
| | | | 1.351(0.4); 1.298(1.0); 1.259(1.6); 1.235(3.6); 1.175(0.4); 1.148(0.4); 0.868(5.8); |
| | | | 0.856(3.3); 0.146(1.6); 0.008(12.6); 0.000(363.3); −0.009(12.5); −0.150(1.6) |
| I-068 | 3.87 | 4.00 | Example I-068: ¹H-NMR(400.0 MHz, d₆-DMSO): δ = |
| | | | 11.011(5.9); 9.865(0.5); 8.772(1.6); 8.757(3.5); 8.742(1.7); 8.568(7.5); 8.564(7.9); |
| | | | 8.451(2.6); 8.388(0.4); 8.384(0.5); 8.317(1.5); 8.201(0.6); 8.196(0.5); 8.135(0.4); |
| | | | 8.116(1.0); 8.097(0.7); 7.988(5.4); 7.983(10.7); 7.979(9.5); 7.967(8.5); |
| | | | 7.911(9.1); 7.890(5.4); 7.860(0.6); 7.854(0.8); 7.839(7.5); 7.821(7.5); 7.806(1.1); |
| | | | 7.799(1.6); 7.788(0.8); 7.770(0.8); 4.688(6.2); 4.674(5.9); 4.633(0.4); 4.617(0.5); |
| | | | 3.702(0.3); 3.682(0.6); 3.625(0.6); 3.406(0.3); 3.363(0.8); 3.326(530.3); |
| | | | 2.891(0.4); 2.731(0.4); 2.694(0.4); 2.676(4.4); 2.671(6.1); 2.666(4.5); 2.662 |
| | | | (2.3); 2.643(0.4); 2.635(0.4); 2.524(16.2); 2.520(24.2); 2.511(323.7); 2.506(668.6); |
| | | | 2.502(896.3); 2.497(662.0); 2.493(325.4); 2.338(2.0); 2.333(4.1); 2.329(5.8); |
| | | | 2.324(4.2); 2.075(2.3); 2.054(1.5); 2.040(2.2); 2.024(1.8); 2.009(0.7); 1.234(0.8); |
| | | | 1.147(0.8); 1.088(0.3); 1.070(0.4); 1.039(0.5); 1.029(0.7); 1.020(0.9); 1.011(0.8); |
| | | | 0.985(0.4); 0.975(0.4); 0.944(0.4); 0.903(0.9); 0.893(1.0); 0.870(16.0); 0.857(8.2); |
| | | | 0.852(7.7); 0.829(0.6); 0.146(0.5); 0.008(3.7); 0.000(129.4); −0.009(4.3); −0.150(0.5) |
| I-069 | 4.05 | 4.16 | Example I-069: ¹H-NMR(400.0 MHz, d₆-DMSO): δ = |
| | | | 10.630(2.7); 8.763(0.8); 8.748(1.7); 8.733(0.8); 8.624(3.5); 8.620(3.6); 8.453(1.1); |
| | | | 8.006(2.4); 7.991(5.1); 7.987(7.0); 7.919(4.1); 7.898(2.7); 7.853(0.4); 7.837(3.6); |
| | | | 7.820(3.6); 7.798(0.8); 5.759(3.0); 4.686(3.1); 4.671(2.8); 3.623(0.3); |
| | | | 3.329(46.4); 2.892(1.1); 2.828(0.4); 2.811(1.0); 2.794(1.4); 2.777(1.1); |
| | | | 2.760(0.4); 2.732(0.9); 2.677(0.4); 2.673(0.6); 2.668(0.4); 2.526(1.5); 2.521(2.3); |
| | | | 2.512(31.8); 2.508(64.7); 2.504(85.7); 2.499(63.1); 2.495(31.1); 2.335(0.4); |
| | | | 2.330(0.5); 2.326(0.4); 1.232(0.5); 1.132(16.0); 1.115(15.8); 1.061(0.6); |
| | | | 1.043(0.3); 0.008(0.4); 0.000(13.0); −0.009(0.4) |
| I-070 | 4.15 | 4.31 | Example I-070: ¹H-NMR(400.0 MHz, d₆-DMSO): δ = |
| | | | 10.588(2.5); 9.850(0.5); 8.744(0.7); 8.729(1.6); 8.714(0.8); 8.573(3.4); 8.570(3.6); |
| | | | 8.407(2.6); 8.317(0.9); 8.036(0.8); 8.014(0.9); 7.947(3.9); 7.943(4.0); 7.915(4.2); |
| | | | 7.910(1.5); 7.898(1.5); 7.893(4.8); 7.850(0.5); 7.835(3.6); 7.817(3.8); |
| | | | 7.795(1.6); 7.546(3.1); 7.526(2.9); 7.512(0.8); 7.346(0.4); 7.339(0.5); 5.757(0.5); |
| | | | 4.679(3.4); 4.665(2.7); 3.344(100.6); 2.891(0.4); 2.820(0.4); 2.803(1.1); |
| | | | 2.786(1.5); 2.769(1.2); 2.752(0.5); 2.732(0.5); 2.680(1.3); 2.676(2.6); 2.671(3.7); |
| | | | 2.666(2.8); 2.524(9.4); 2.520(13.9); 2.511(190.3); 2.506(392.8); 2.502(527.2); |
| | | | 2.497(389.9); 2.493(191.7); 2.450(0.4); 2.338(1.1); 2.333(2.4); 2.329(3.4); |
| | | | 2.324(2.5); 1.569(0.3); 1.520(0.7); 1.234(1.0); 1.187(0.4); 1.174(0.7); 1.158(0.9); |
| | | | 1.142(0.8); 1.127(16.0); 1.110(15.8); 1.089(0.8); 1.065(0.6); 1.061(0.7); |
| | | | 1.048(0.6); 1.034(0.6); 1.026(0.6); 1.021(0.7); 0.962(0.3); 0.955(0.3); 0.936(0.4); |
| | | | 0.008(0.4); 0.000(11.9); −0.009(0.3) |
| I-071 | 4.08 | 4.23 | Example I-071: ¹H-NMR(400.0 MHz, d₆-DMSO): δ = |
| | | | 10.633(3.3); 8.971(0.9); 8.956(2.0); 8.941(1.0); 8.636(4.3); 8.532(0.8); 8.023(7.3); |
| | | | 8.020(7.3); 8.003(6.3); 7.990(1.9); 7.927(4.9); 7.906(3.4); 7.553(1.2); 7.539(1.4); |
| | | | 7.530(1.5); 7.516(1.4); 5.759(3.6); 5.756(3.0); 4.740(0.4); 4.725(0.4); |
| | | | 4.664(3.5); 4.649(3.6); 3.329(25.3); 2.892(0.5); 2.851(0.4); 2.835(1.1); 2.817(1.5); |
| | | | 2.801(1.2); 2.784(0.5); 2.732(0.4); 2.674(0.4); 2.504(60.1); 2.331(0.4); 1.230(0.3); |
| | | | 1.144(16.0); 1.127(16.0); 1.061(0.5); 1.043(0.3); 0.146(0.3); 0.008(2.6); |
| | | | 0.006(2.8); 0.000(68.7); −0.002(54.3); −0.150(0.4) |
| I-072 | 4.04 | 4.23 | Example I-072: ¹H-NMR(400.0 MHz, d₆-DMSO): δ = |
| | | | 10.612(3.6); 9.063(2.3); 8.634(4.5); 8.548(0.4); 8.025(8.3); 8.007(5.8); 7.928(5.6); |
| | | | 7.909(4.1); 7.418(5.0); 7.395(4.9); 5.760(2.7); 5.758(2.6); 4.766(0.4); 4.752(0.4); |
| | | | 4.667(4.6); 4.653(4.7); 3.335(20.6); 3.330(23.0); 2.893(0.5); 2.854(0.6); |
| | | | 2.838(1.2); 2.822(1.7); 2.806(1.4); 2.733(0.5); 2.673(0.5); 2.505(70.0); 2.332(0.5); |
| | | | 1.370(0.7); 1.230(0.5); 1.147(15.7); 1.130(16.0); 1.060(0.7); 0.000(0.3) |
| I-073 | 4.21 | 4.35 | Example I-073: ¹H-NMR(400.0 MHz, d₆-DMSO): δ = |
| | | | 10.571(2.6); 9.061(0.7); 9.045(1.6); 9.029(0.7); 8.585(3.4); 8.581(3.6); 8.503(1.2); |
| | | | 8.094(0.3); 8.071(0.4); 7.983(4.0); 7.979(4.1); 7.944(0.4); 7.937(4.3); 7.932(1.5); |
| | | | 7.920(1.4); 7.915(5.1); 7.907(0.6); 7.556(2.9); 7.536(2.6); 7.432(0.6); |
| | | | 7.413(3.9); 7.391(3.7); 4.759(0.4); 4.743(0.4); 4.660(2.9); 4.644(2.9); 3.337(37.6); |
| | | | 2.847(0.4); 2.830(1.0); 2.813(1.4); 2.796(1.0); 2.779(0.4); 2.680(0.4); 2.676(0.8); |
| | | | 2.671(1.1); 2.667(0.8); 2.662(0.4); 2.525(2.5); 2.520(3.9); 2.511(59.7); |
| | | | 2.507(124.9); 2.502(168.4); 2.498(123.7); 2.493(60.7); 2.338(0.4); 2.334(0.8); |
| | | | 2.329(1.1); 2.324(0.8); 2.320(0.4); 1.141(16.0); 1.124(15.8); 0.146(1.1); |
| | | | 0.014(0.6); 0.008(8.4); 0.000(255.6); −0.009(9.8); −0.150(1.2) |
| I-074 | 3.90 | 4.05 | Example I-074: ¹H-NMR(400.0 MHz, d₆-DMSO): δ = |
| | | | 11.021(6.9); 9.988(0.4); 8.976(1.9); 8.961(4.1); 8.945(2.0); 8.585(7.8); 8.582(8.5); |
| | | | 8.531(1.6); 8.165(0.4); 8.145(0.5); 8.043(0.4); 8.028(3.4); 8.015(12.2); 8.012(12.1); |
| | | | 8.006(9.8); 7.991(6.1); 7.986(9.9); 7.953(0.3); 7.919(10.0); 7.898(6.3); |
| | | | 7.560(2.5); 7.545(2.8); 7.536(2.9); 7.521(2.7); 7.504(0.4); 5.758(7.3); 4.740(0.7); |
| | | | 4.725(0.7); 4.669(6.8); 4.653(6.8); 3.343(33.5); 2.892(1.1); 2.732(1.0); 2.677(0.6); |
| | | | 2.673(0.8); 2.668(0.6); 2.551(0.4); 2.508(96.7); 2.504(127.8); 2.499(98.1); |
| | | | 2.335(0.6); 2.330(0.8); 2.326(0.6); 2.099(0.6); 2.083(1.7); 2.075(1.4); 2.068(2.7); |
| | | | 2.052(1.8); 2.037(0.7); 1.231(0.4); 0.937(0.3); 0.918(0.3); 0.881(16.0); 0.870(9.1); |
| | | | 0.862(7.8); 0.838(0.5); 0.146(0.9); 0.008(9.1); 0.000(183.1); −0.009(10.6); −0.150(0.9) |
| I-075 | 3.47 | 3.58 | Example I-075: ¹H-NMR(400.0 MHz, d₆-DMSO): δ = |
| | | | 10.543(2.6); 9.256(3.2); 8.589(3.5); 8.586(3.7); 7.951(4.3); 7.947(4.4); 7.943(4.7); |
| | | | 7.938(1.6); 7.926(1.5); 7.921(5.0); 7.914(0.6); 7.557(3.0); 7.537(2.6); 5.758(0.4); |
| | | | 3.622(1.7); 3.329(78.3); 3.327(73.2); 2.839(0.4); 2.822(1.0); 2.805(1.5); |
| | | | 2.788(1.1); 2.771(0.4); 2.676(0.6); 2.671(0.8); 2.667(0.6); 2.525(2.0); 2.511 |
| | | | (43.7); 2.507(90.3); 2.502(121.1); 2.498(89.3); 2.493(44.2); 2.333(0.6); 2.329(0.8); |
| | | | 2.324(0.6); 1.632(1.2); 1.617(2.9); 1.610(3.1); 1.597(1.4); 1.357(1.4); 1.343(2.9); |
| | | | 1.336(3.1); 1.322(1.1); 1.138(16.0); 1.121(15.8); 1.060(2.7); 1.043(1.1); |
| | | | 1.025(1.0); 0.146(0.4); 0.008(3.1); 0.000(96.2); −0.009(3.4); −0.150(0.4) |
| I-076 | 3.29 | 3.39 | Example I-076: ¹H-NMR(400.0 MHz, d₆-DMSO): δ = |
| | | | 10.949(4.6); 10.015(0.3); 9.704(0.4); 9.268(5.7); 8.529(5.9); 8.525(6.3); 8.505 |
| | | | (0.7); 8.402(0.4); 8.399(0.4); 8.317(1.3); 8.201(0.4); 8.197(0.4); 8.089(0.5); |
| | | | 8.066(0.6); 7.943(6.9); 7.940(7.2); 7.927(7.4); 7.922(2.7); 7.910(2.5); 7.905(8.6); |
| | | | 7.843(0.4); 7.570(0.5); 7.549(5.5); 7.529(4.6); 3.702(1.2); 3.681(1.1); 3.646(0.5); |
| | | | 3.625(0.6); 3.507(2.3); 3.407(0.3); 3.325(543.1); 2.891(2.1); 2.730(1.7); |
| | | | 2.675(4.1); 2.671(5.8); 2.666(4.3); 2.624(0.3); 2.620(0.4); 2.524(13.7); 2.519(21.2); |
| | | | 2.511(310.3); 2.506(649.9); 2.502(877.1); 2.497(656.8); 2.493(330.6); 2.415(0.5); |
| | | | 2.333(4.2); 2.328(5.8); 2.324(4.4); 2.285(0.4); 2.267(0.4); 2.206(0.4); |
| | | | 2.197(0.7); 2.189(0.5); 2.179(1.1); 2.160(0.6); 2.091(0.5); 2.076(1.5); 2.061(2.0); |
| | | | 2.045(1.9); 2.030(1.0); 2.022(0.9); 2.004(0.6); 1.971(0.6); 1.958(0.5); 1.939(0.4); |
| | | | 1.728(0.4); 1.722(0.4); 1.632(2.6); 1.618(5.1); 1.611(5.6); 1.597(2.5); |
| | | | 1.576(0.5); 1.569(0.5); 1.551(1.8); 1.505(0.6); 1.491(0.6); 1.474(0.6); 1.458(0.5); |
| | | | 1.409(0.5); 1.398(0.6); 1.389(0.7); 1.382(0.5); 1.357(3.0); 1.344(5.5); 1.337 |
| | | | (5.8); 1.322(2.3); 1.235(16.0); 1.172(0.5); 1.163(0.4); 1.145(0.6); 1.120(0.5); |
| | | | 1.104(0.4); 1.061(0.6); 1.044(1.0); 1.033(1.3); 1.026(1.3); 0.982(0.5); 0.972(0.4); |
| | | | 0.963(0.5); 0.951(0.5); 0.936(0.7); 0.924(0.7); 0.916(0.9); 0.896(1.2); 0.871(14.5); |
| | | | 0.854(11.0); 0.836(2.0); 0.810(0.7); 0.795(0.3); 0.146(2.7); 0.008(20.4); |
| | | | 0.000(667.2); −0.009(25.6); −0.069(0.3); −0.150(2.9) |
| I-077 | 3.79 | 3.90 | Example I-077: ¹H-NMR(400.0 MHz, d₆-DMSO): δ = |
| | | | 11.033(6.5); 8.938(1.9); 8.922(3.9); 8.907(1.9); 8.693(1.0); 8.681(1.0); 8.586(8.4); |
| | | | 8.582(8.2); 8.317(0.8); 8.017(9.7); 8.013(9.9); 8.006(6.2); 7.985(9.2); 7.919(9.6); |
| | | | 7.898(6.8); 7.891(4.6); 7.869(3.8); 7.866(3.8); 7.718(3.5); 7.715(3.4); |
| | | | 7.698(4.8); 7.695(4.6); 7.607(3.5); 7.588(5.3); 7.568(2.3); 5.757(1.8); 4.683(6.6); |
| | | | 4.668(6.7); 3.623(0.6); 3.511(0.5); 3.450(1.1); 3.344(591.9); 3.250(1.0); |
| | | | 3.242(0.8); 3.198(0.4); 3.184(0.4); 3.168(0.9); 2.891(0.8); 2.731(0.7); 2.676(3.7); |
| | | | 2.671(4.9); 2.667(3.6); 2.524(16.0); 2.511(318.1); 2.507(603.0); 2.502(768.1); |
| | | | 2.498(567.2); 2.431(0.6); 2.333(3.6); 2.329(4.9); 2.325(3.6); 2.086(0.6); |
| | | | 2.075(2.1); 2.055(2.6); 2.040(1.8); 2.025(0.7); 1.235(0.5); 1.060(0.6); 1.043(0.5); |
| | | | 1.029(0.7); 0.937(0.4); 0.918(0.3); 0.878(16.0); 0.867(9.0); 0.860(7.7); 0.000(7.4) |
| I-078 | 3.96 | 3.99 | Example I-078: ¹H-NMR(400.0 MHz, d₆-DMSO): δ = |
| | | | 10.647(3.0); 8.934(0.8); 8.918(1.7); 8.902(0.8); 8.639(3.6); 8.635(3.7); 8.318(0.3); |
| | | | 8.024(6.3); 8.020(5.6); 8.002(4.0); 7.927(4.2); 7.906(2.8); 7.893(1.7); 7.889(1.8); |
| | | | 7.868(1.6); 7.864(1.7); 7.716(1.5); 7.713(1.5); 7.696(2.0); 1.693(2.1); |
| | | | 7.601(1.4); 7.582(2.2); 7.563(1.0); 4.680(2.9); 4.664(2.9); 3.347(39.7); 2.841(0.4); |
| | | | 2.824(1.1); 2.806(1.4); 2.789(1.1); 2.772(0.5); 2.676(0.7); 2.672(0.9); 2.667(0.7); |
| | | | 2.525(2.2); 2.512(52.4); 2.507(107.0); 2.503(141.8); 2.498(104.9); 2.334(0.7); |
| | | | 2.330(1.0); 2.325(0.7); 1.140(16.0); 1.123(15.8); 0.008(0.6); 0.000(19.4); −0.009(0.8) |
| I-079 | 3.91 | 4.03 | Example I-079: ¹H-NMR(400.0 MHz, d₆-DMSO): δ = |
| | | | 10.997(6.2); 8.920(1.8); 8.904(3.7); 8.889(1.8); 8.537(7.6); 8.534(7.8); 8.318(0.9); |
| | | | 8.181(0.4); 8.177(0.3); 8.075(0.4); 8.053(0.4); 7.975(8.4); 7.971(8.3); 7.916(9.2); |
| | | | 7.911(3.8); 7.894(13.8); 7.869(3.6); 7.865(3.8); 7.717(3.3); 7.714(3.3); |
| | | | 7.697(4.4); 7.694(4.5); 7.604(2.9); 7.584(4.7); 7.565(2.7); 7.547(7.0); 7.526(6.2); |
| | | | 4.678(6.3); 4.663(6.3); 3.423(0.4); 3.330(243.8); 2.676(2.2); 2.672(2.9); |
| | | | 2.667(2.2); 2.578(0.3); 2.557(0.8); 2.525(10.0); 2.511(171.3); 2.507(334.5); |
| | | | 2.503(436.3); 2.498(329.6); 2.334(2.1); 2.329(2.9); 2.325(2.2); 2.081(0.6); |
| | | | 2.065(1.6); 2.050(2.5); 2.035(1.9); 2.020(0.8); 1.234(3.0); 1.055(0.5); 1.043(0.5); |
| | | | 1.037(0.6); 1.025(0.5); 0.934(0.3); 0.925(0.5); 0.917(0.5); 0.906(0.7); 0.872(16.0); |
| | | | 0.860(9.0); 0.854(8.4); 0.008(2.0); 0.000(48.8) |
| I-080 | 3.87 | 4.01 | Example I-080: ¹H-NMR(400.0 MHz, d₆-DMSO): δ = |
| | | | 10.968(1.8); 9.024(0.5); 9.009(1.0); 8.994(0.4); 8.530(2.1); 8.526(2.3); 8.317(5.3); |
| | | | 7.995(0.3); 7.978(2.5); 7.974(2.6); 7.934(1.1); 7.919(3.1); 7.914(2.5); 7.897(4.3); |
| | | | 7.802(0.4); 7.547(2.1); 7.528(2.0); 7.505(1.1); 7.479(1.2); 7.402(1.1); |
| | | | 7.379(1.1); 4.663(1.8); 4.647(1.8); 3.681(0.7); 3.650(0.4); 3.627(0.4); 3.617(0.4); |
| | | | 3.589(0.4); 3.558(0.3); 3.498(0.4); 3.476(0.5); 3.469(0.5); 3.430(0.6); 3.424 |
| | | | (0.7); 3.388(1.3); 3.328(2978.8); 3.276(0.9); 3.251(0.4); 3.242(0.5); 2.809(0.4); |
| | | | 2.795(0.4); 2.779(0.4); 2.747(0.3); 2.715(0.5); 2.694(0.7); 2.680(5.2); 2.676(11.3); |
| | | | 2.671(16.0); 2.666(12.1); 2.662(6.1); 2.524(40.8); 2.520(59.1); 2.511(784.8); |
| | | | 2.506(1642.7); 2.502(2247.0); 2.497(1706.4); 2.493(863.9); 2.421(0.7); |
| | | | 2.381(0.4); 2.375(0.4); 2.338(4.7); 2.333(10.8); 2.329(15.3); 2.324(11.3); |
| | | | 2.320(5.5); 2.070(0.5); 2.053(0.7); 2.041(0.7); 1.234(1.2); 1.064(0.5); 1.056(0.5); |
| | | | 1.044(0.9); 1.024(0.5); 0.992(0.4); 0.976(0.4); 0.961(0.4); 0.956(0.4); 0.946(0.4); |
| | | | 0.930(0.5); 0.925(0.7); 0.907(0.7); 0.873(5.2); 0.861(3.0); 0.853(3.0); 0.008 |
| | | | (0.5); 0.000(16.3) |
| I-081 | 4.09 | 4.22 | Example I-081: ¹H-NMR(400.0 MHz, d₆-DMSO): δ = |
| | | | 10.602(2.9); 8.917(0.8); 8.901(1.8); 8.885(0.8); 8.588(3.5); 8.584(3.7); 8.316(1.4); |
| | | | 7.980(3.8); 7.976(4.0); 7.932(4.2); 7.915(1.6); 7.910(5.0); 7.886(1.8); 7.862(1.8); |
| | | | 7.714(1.6); 7.711(1.6); 7.694(2.0); 7.691(2.1); 7.597(1.4); 7.578(2.2); |
| | | | 7.554(3.6); 7.533(2.9); 4.674(2.9); 4.658(2.9); 3.577(0.4); 3.503(0.4); 3.482(0.5); |
| | | | 3.449(0.6); 3.427(0.7); 3.408(1.0); 3.384(1.4); 3.328(2040.7); 3.278(0.7); |
| | | | 3.269(0.5); 2.833(0.4); 2.815(1.0); 2.798(1.5); 2.781(1.2); 2.764(0.5); 2.675(5.5); |
| | | | 2.671(7.6); 2.666(5.9); 2.620(0.5); 2.598(0.5); 2.592(0.6); 2.524(19.0); 2.506 |
| | | | (900.6); 2.502(1204.7); 2.497(907.7); 2.414(0.4); 2.406(0.4); 2.333(5.3); |
| | | | 2.328(7.5); 2.324(5.6); 2.074(1.0); 1.520(0.4); 1.234(0.7); 1.157(0.4); 1.136(16.0); |
| | | | 1.119(16.0); 1.100(0.6); 1.059(0.4); 1.043(0.9); 1.027(1.0); 0.000(15.3) |
| I-082 | 4.01 | 4.12 | Example I-082: ¹H-NMR(400.0 MHz, d₆-DMSO): δ = |
| | | | 10.976(5.9); 8.758(1.6); 8.743(3.5); 8.728(1.8); 8.601(0.4); 8.520(7.1); 8.516(7.4); |
| | | | 8.407(0.8); 8.344(0.5); 8.317(3.1); 8.141(0.5); 8.056(0.6); 8.035(0.8); 8.015(0.3); |
| | | | 7.940(7.9); 7.937(7.9); 7.898(8.6); 7.876(10.0); 7.853(0.9); 7.837(7.3); |
| | | | 7.820(7.3); 7.805(0.9); 7.798(0.9); 7.788(0.7); 7.771(0.5); 7.539(6.7); 7.519(5.9); |
| | | | 7.438(0.4); 7.426(0.5); 7.314(0.3); 4.683(6.3); 4.669(6.2); 4.627(0.5); 4.611(0.5); |
| | | | 3.709(0.3); 3.626(0.5); 3.611(0.4); 3.592(0.4); 3.564(0.4); 3.524(0.4); |
| | | | 3.496(0.5); 3.468(0.7); 3.421(1.0); 3.417(1.0); 3.335(1332.3); 3.242(0.4); |
| | | | 3.168(0.4); 2.777(0.4); 2.741(0.4); 2.697(0.6); 2.676(7.4); 2.671(10.1); 2.667(7.7); |
| | | | 2.603(1.1); 2.588(1.1); 2.565(2.0); 2.507(1156.1); 2.502(1503.9); 2.498(1119.3); |
| | | | 2.334(7.0); 2.329(9.6); 2.324(7.3); 2.063(0.6); 2.049(1.8); 2.033(2.3); 2.018(1.9); |
| | | | 2.003(0.8); 1.988(0.4); 1.527(0.4); 1.487(0.3); 1.234(1.9); 1.016(1.0); |
| | | | 1.005(0.8); 0.919(0.5); 0.900(0.8); 0.864(16.0); 0.851(9.3); 0.146(0.5); |
| | | | 0.008(4.0); 0.000(100.7); −0.008(4.7); −0.149(0.4) |
| I-083 | 4.05 | 4.22 | Example I-083: ¹H-NMR(400.0 MHz, d₆-DMSO): δ = |
| | | | 10.996(2.1); 8.608(0.6); 8.593(1.2); 8.578(0.7); 8.530(2.5); 8.526(2.7); 8.317(0.5); |
| | | | 7.987(2.8); 7.983(3.0); 7.914(3.1); 7.892(3.6); 7.546(2.3); 7.525(2.1); 7.311(3.0); |
| | | | 7.289(3.4); 6.940(0.4); 6.934(3.6); 6.912(3.3); 4.500(2.6); 4.485(2.6); |
| | | | 3.740(16.0); 3.721(0.5); 3.325(144.8); 2.675(1.3); 2.671(1.8); 2.666(1.4); |
| | | | 2.524(4.7); 2.506(212.3); 2.502(282.1); 2.497(211.3); 2.333(1.3); 2.328(1.9); |
| | | | 2.324(1.4); 2.013(0.6); 1.999(0.9); 1.984(0.6); 1.234(0.6); 0.858(4.9); 0.847(2.9); |
| | | | 0.838(2.4); 0.146(1.0); 0.008(7.4); 0.000(219.0); −0.008(9.7); −0.150(1.0) |
| I-084 | 3.85 | 3.98 | Example I-084: ¹H-NMR(400.0 MHz, d₆-DMSO): δ = |
| | | | 11.019(7.6); 9.034(2.0); 9.018(4.4); 9.002(2.1); 8.584(9.4); 8.581(10.0); 8.435 |
| | | | (0.4); 8.431(0.5); 8.317(0.4); 8.242(12.0); 8.237(5.1); 8.220(13.3); 8.194(0.7); |
| | | | 8.155(0.4); 8.134(0.4); 8.033(10.3); 8.029(10.8); 8.010(6.7); 7.990(10.7); |
| | | | 7.921(11.1); 7.900(7.1); 7.634(10.9); 7.612(10.9); 7.590(0.6); 5.757(11.9); |
| | | | 4.712(8.5); 4.697(8.5); 4.654(0.4); 4.638(0.4); 3.329(140.7); 2.676(1.0); 2.672(1.4); |
| | | | 2.667(1.1); 2.525(3.1); 2.511(78.8); 2.507(160.1); 2.503(213.2); 2.498(160.3); |
| | | | 2.494(81.4); 2.334(1.1); 2.330(1.4); 2.325(1.1); 2.084(0.7); 2.070(1.7); 2.066(1.9); |
| | | | 2.060(1.5); 2.053(3.2); 2.046(1.5); 2.036(2.2); 2.022(1.0); 1.233(0.5); 1.051 |
| | | | (0.5); 1.042(0.7); 1.032(0.6); 0.930(0.7); 0.920(0.6); 0.911(0.8); 0.903(0.9); |
| | | | 0.876(16.0); 0.867(10.1); 0.856(8.1); 0.834(0.7); 0.146(0.9); 0.008(6.6); |
| | | | 0.000(193.6); −0.008(8.2); −0.150(0.9) |
| I-085 | 3.97 | 4.11 | Example I-085: ¹H-NMR(400.0 MHz, d₆-DMSO): δ = |
| | | | 10.979(6.8); 9.016(1.9); 9.000(4.0); 8.984(1.9); 8.534(8.1); 8.530(8.5); 8.317(0.8); |
| | | | 8.240(10.3); 8.218(11.5); 8.193(0.5); 8.173(0.3); 8.053(0.3); 7.989(8.9); |
| | | | 7.986(9.2); 7.920(9.9); 7.898(11.4); 7.630(9.7); 7.609(9.4); 7.587(0.5); 7.548(7.6); |
| | | | 7.528(6.7); 4.706(7.5); 4.691(7.5); 3.325(281.8); 2.676(2.2); 2.671(3.0); |
| | | | 2.667(2.3); 2.506(338.3); 2.502(442.4); 2.498(333.1); 2.432(0.7); 2.333(2.1); |
| | | | 2.329(2.8); 2.325(2.1); 2.075(0.9); 2.059(1.7); 2.053(1.4); 2.045(2.8); 2.029(1.9); |
| | | | 2.014(0.8); 1.234(0.7); 1.044(0.5); 1.037(0.6); 0.926(0.5); 0.915(0.6); 0.899(1.1); |
| | | | 0.870(16.0); 0.859(9.1); 0.850(7.7); 0.146(1.5); 0.008(13.7); |
| | | | 0.000(328.7); −0.008(13.8); −0.150(1.5) |
| I-086 | 4.02 | 4.15 | Example I-086: ¹H-NMR(400.0 MHz, d₆-DMSO): δ = |
| | | | 10.985(6.3); 10.049(0.5); 10.034(1.2); 10.018(0.6); 8.963(1.7); 8.948(3.8); |
| | | | 8.932(1.8); 8.804(1.0); 8.792(0.9); 8.536(7.9); 8.532(8.5); 8.484(6.0); 8.319(0.4); |
| | | | 8.289(0.4); 8.083(1.7); 8.077(0.7); 8.066(0.6); 8.061(2.0); 8.053(0.6); 8.040(1.0); |
| | | | 8.027(3.9); 8.014(3.5); 8.004(4.2); 7.991(3.3); 7.973(9.2); 7.969(9.6); 7.956 |
| | | | (0.4); 7.924(1.0); 7.917(9.9); 7.912(3.6); 7.900(3.4); 7.895(11.7); 7.887(1.5); |
| | | | 7.829(0.6); 7.814(0.8); 7.795(0.6); 7.556(3.8); 7.547(8.3); 7.527(7.4); 7.518(3.7); |
| | | | 7.337(0.4); 7.239(0.4); 7.232(0.3); 7.202(0.4); 5.758(14.3); 4.731(1.9); 4.716 |
| | | | (1.9); 4.664(6.1); 4.648(6.2); 4.619(0.4); 3.683(0.8); 3.627(0.7); 3.623(0.5); |
| | | | 3.618(0.5); 3.601(0.5); 3.447(4.6); 3.185(0.5); 3.163(0.4); 3.143(0.4); 2.892(1.3); |
| | | | 2.732(1.1); 2.682(0.4); 2.677(0.8); 2.673(1.2); 2.668(0.9); 2.664(0.4); 2.526(2.6); |
| | | | 2.521(3.9); 2.513(66.5); 2.508(139.7); 2.504(188.4); 2.499(139.3); 2.495 |
| | | | (68.9); 2.339(0.5); 2.335(0.9); 2.331(1.3); 2.326(1.0); 2.321(0.5); 2.093(0.6); |
| | | | 2.077(1.7); 2.069(1.2); 2.062(2.5); 2.046(1.9); 2.032(0.8); 1.320(0.5); 1.301(0.5); |
| | | | 1.262(0.4); 1.247(0.4); 1.232(0.8); 1.123(0.3); 1.061(0.5); 1.048(0.6); 1.040(0.7); |
| | | | 1.029(0.7); 0.964(0.3); 0.956(1.2); 0.938(2.4); 0.928(0.7); 0.919(1.4); 0.908(0.7); |
| | | | 0.899(0.7); 0.875(16.0); 0.864(8.2); 0.856(7.4); 0.833(0.6); 0.146(1.4); |
| | | | 0.008(10.2); 0.000(300.5); −0.009(11.2); −0.150(1.4) |
| I-087 | 4.15 | 4.20 | Example I-087: ¹H-NMR(400.0 MHz, d₆-DMSO): δ = |
| | | | 10.688(3.7); 8.755(2.1); 8.735(2.2); 8.652(3.9); 8.649(4.7); 8.009(3.3); 7.988(5.2); |
| | | | 7.970(4.3); 7.967(5.0); 7.917(5.3); 7.896(3.5); 7.845(4.8); 7.825(6.1); 7.633(5.6); |
| | | | 7.613(4.8); 5.758(1.1); 5.255(1.1); 5.237(1.7); 5.218(1.1); 3.622(2.7); |
| | | | 3.590(0.5); 3.579(0.6); 3.327(137.2); 3.254(0.4); 2.853(0.5); 2.837(1.2); 2.820 |
| | | | (1.7); 2.803(1.3); 2.786(0.5); 2.671(1.6); 2.506(179.8); 2.502(240.1); 2.498(199.1); |
| | | | 2.329(1.6); 1.629(0.4); 1.565(7.1); 1.548(7.1); 1.521(1.1); 1.506(0.5); 1.239(1.2); |
| | | | 1.151(16.0); 1.134(15.8); 1.085(0.4); 1.060(4.9); 1.042(2.7); 1.035(1.6); |
| | | | 1.026(2.9); 0.146(0.7); 0.008(7.4); 0.000(142.3); −0.150(0.7) |
| I-088 | 5.54 | 4.37 | Example I-088: ¹H-NMR(400.0 MHz, d₆-DMSO): δ = |
| | | | 11.040(2.6); 8.548(2.3); 8.334(1.6); 8.323(1.6); 8.317(1.5); 7.993(2.5); 7.986(4.6); |
| | | | 7.983(4.4); 7.972(3.9); 7.905(4.1); 7.884(2.6); 7.765(0.4); 7.743(0.5); 7.723(2.9); |
| | | | 7.702(3.9); 7.679(6.2); 7.658(7.2); 7.635(0.4); 7.597(3.7); 7.577(2.7); |
| | | | 7.564(0.5); 7.544(0.4); 7.424(6.6); 7.404(5.7); 5.534(0.4); 4.051(16.0); 3.925(5.6); |
| | | | 3.689(1.5); 3.325(438.9); 2.939(0.5); 2.929(0.6); 2.921(1.0); 2.910(1.1); |
| | | | 2.902(0.7); 2.892(0.5); 2.710(0.5); 2.675(3.6); 2.671(5.0); 2.666(3.7); 2.541(130.8); |
| | | | 2.524(11.9); 2.511(281.6); 2.506(576.0); 2.502(764.2); 2.497(559.5); 2.493(273.2); |
| | | | 2.367(0.7); 2.333(3.6); 2.328(4.9); 2.324(3.6); 1.863(0.6); 1.234(0.6); |
| | | | 0.809(0.7); 0.796(1.8); 0.791(2.6); 0.779(2.5); 0.773(2.1); 0.762(0.9); 0.646 |
| | | | (0.9); 0.635(2.6); 0.629(2.3); 0.620(2.1); 0.607(0.6); 0.146(0.5); 0.008(3.7); |
| | | | 0.000(123.2); −0.009(4.2); −0.024(0.4); −0.150(0.5) |
| I-089 | 3.39 | 3.51 | Example I-089: ¹H-NMR(400.0 MHz, d₆-DMSO): δ = |
| | | | 10.963(5.6); 8.508(7.1); 8.505(7.5); 8.311(3.3); 8.300(3.3); 7.919(7.9); 7.915(8.4); |
| | | | 7.906(8.9); 7.901(3.2); 7.889(3.1); 7.884(10.2); 7.877(1.1); 7.542(6.4); 7.521(5.7); |
| | | | 3.326(207.2); 2.940(0.4); 2.930(1.0); 2.920(1.4); 2.912(2.2); 2.901(2.2); |
| | | | 2.893(1.4); 2.883(1.1); 2.873(0.4); 2.675(1.2); 2.671(1.7); 2.667(1.3); 2.524(4.4); |
| | | | 2.506(185.3); 2.502(247.8); 2.498(186.4); 2.333(1.2); 2.329(1.6); 2.324(1.2); |
| | | | 2.056(0.5); 2.041(1.9); 2.025(2.4); 2.010(2.1); 1.994(0.6); 0.869(16.0); |
| | | | 0.853(11.6); 0.803(1.5); 0.790(4.0); 0.785(5.6); 0.772(5.2); 0.767(4.5); 0.755(1.8); |
| | | | 0.637(1.9); 0.626(5.5); 0.620(5.0); 0.616(4.8); 0.610(4.6); 0.598(1.4); 0.000(9.8) |
| I-090 | 3.72 | 3.79 | Example I-090: ¹H-NMR(400.0 MHz, d₆-DMSO): δ = |
| | | | 10.992(5.6); 9.049(2.3); 8.790(0.3); 8.770(5.1); 8.767(5.0); 8.554(7.8); 8.551(8.0); |
| | | | 8.525(0.8); 8.316(0.8); 8.082(0.4); 8.063(0.3); 8.043(2.5); 8.041(2.7); 8.023(8.2); |
| | | | 8.021(7.9); 8.007(5.5); 8.002(6.5); 7.999(10.1); 7.995(9.2); 7.987(2.1); |
| | | | 7.982(2.0); 7.939(5.3); 7.917(14.5); 7.891(12.1); 7.870(4.8); 4.688(6.9); 4.673(7.0); |
| | | | 3.625(0.4); 3.506(0.4); 3.487(0.4); 3.456(0.4); 3.435(0.6); 3.431(0.6); 3.407(1.0); |
| | | | 3.344(488.3); 3.340(570.2); 3.338(541.1); 3.333(536.4); 3.231(0.4); |
| | | | 3.225(0.3); 2.676(2.8); 2.672(3.9); 2.667(2.9); 2.597(0.3); 2.587(0.3); 2.525(9.2); |
| | | | 2.511(212.0); 2.507(430.1); 2.503(578.0); 2.498(437.3); 2.494(222.2); 2.421(0.4); |
| | | | 2.334(2.7); 2.329(3.7); 2.325(2.8); 2.082(0.5); 2.068(1.7); 2.053(2.3); |
| | | | 2.037(2.0); 2.023(0.8); 1.552(0.4); 1.235(2.8); 1.216(0.4); 1.050(0.4); 1.034(0.5); |
| | | | 0.997(0.4); 0.977(0.3); 0.917(0.6); 0.906(0.6); 0.873(16.0); 0.860(9.1); 0.856(8.6); |
| | | | 0.807(0.3); 0.146(1.8); 0.028(0.4); 0.008(13.6); 0.000(418.3); −0.008(17.1); −0.150(1.8) |
| I-091 | 3.88 | 4.01 | Example I-091: ¹H-NMR(400.0 MHz, d₆-DMSO): δ = |
| | | | 10.990(6.0); 8.517(7.2); 8.513(7.1); 8.421(1.7); 8.405(3.5); 8.390(1.7); 8.317(0.5); |
| | | | 7.924(8.2); 7.920(7.9); 7.910(1.1); 7.903(9.1); 7.886(3.3); 7.881(10.3); 7.874(1.1); |
| | | | 7.773(6.1); 7.711(3.2); 7.691(3.9); 7.638(2.7); 7.618(3.9); 7.545(10.6); |
| | | | 7.525(11.3); 7.506(2.4); 3.657(1.8); 3.640(4.3); 3.623(4.3); 3.606(2.0); 3.328(139.2); |
| | | | 3.175(0.5); 3.162(0.5); 2.972(3.5); 2.954(6.2); 2.936(3.2); 2.676(1.2); |
| | | | 2.672(1.6); 2.668(1.2); 2.525(4.3); 2.511(85.9); 2.507(170.6); 2.503(225.1); |
| | | | 2.498(166.4); 2.334(1.1); 2.330(1.5); 2.325(1.1); 2.075(1.4); 2.070(0.6); 2.054 |
| | | | (1.7); 2.039(2.3); 2.023(1.9); 2.008(0.6); 1.364(0.3); 1.234(1.3); 0.874(16.0); |
| | | | 0.860(8.9); 0.857(8.6); 0.146(0.8); 0.008(6.0); 0.000(173.8); −0.008(6.3); −0.150(0.8) |
| I-092 | 3.67 | 3.81 | Example I-092: ¹H-NMR(400.0 MHz, d₆-DMSO): δ = |
| | | | 10.992(2.2); 8.761(0.6); 8.746(1.3); 8.731(0.7); 8.539(2.7); 8.536(2.9); 8.528(0.3); |
| | | | 8.073(0.4); 8.051(0.4); 8.022(3.4); 8.020(3.5); 8.013(3.2); 8.009(3.3); 7.988(0.4); |
| | | | 7.928(0.4); 7.921(3.4); 7.916(1.3); 7.904(1.2); 7.899(4.0); 7.891(0.5); |
| | | | 7.752(1.9); 7.731(2.1); 7.614(2.5); 7.549(2.7); 7.529(2.2); 7.182(1.7); 7.179(1.7); |
| | | | 7.161(1.6); 7.158(1.6); 4.722(2.6); 4.706(2.6); 4.032(16.0); 4.006(1.6); 3.978(0.6); |
| | | | 3.626(0.4); 3.332(53.3); 3.169(0.6); 2.677(0.3); 2.672(0.5); 2.668(0.3); |
| | | | 2.525(1.1); 2.512(26.6); 2.507(54.8); 2.503(73.5); 2.498(55.6); 2.494(28.6); |
| | | | 2.334(0.4); 2.330(0.5); 2.325(0.4); 2.076(1.7); 2.018(0.3); 2.007(0.7); 2.001(0.5); |
| | | | 1.994(0.9); 1.981(0.6); 1.977(0.6); 1.233(0.4); 1.033(0.4); 1.026(0.5); 1.015(0.4); |
| | | | 0.908(0.5); 0.897(0.5); 0.889(0.5); 0.880(0.5); 0.853(4.3); 0.846(3.1); |
| | | | 0.833(2.3); 0.146(0.4); 0.008(3.0); 0.000(87.2); −0.009(3.8); −0.150(0.4) |
| I-093 | 4.15 | 4.28 | Example I-093: ¹H-NMR(400.0 MHz, d₆-DMSO): δ = |
| | | | 11.022(5.9); 11.007(0.4); 9.705(11.3); 9.691(1.4); 9.451(0.4); 8.908(1.6); |
| | | | 8.892(3.5); 8.876(1.6); 8.570(6.7); 8.567(6.8); 8.427(0.8); 8.423(0.8); 8.317(0.9); |
| | | | 8.210(0.9); 8.206(0.8): 8.092(1.0); 8.072(1.2); 8.050(8.5); 8.029(16.0); 8.015(1.2); |
| | | | 8.010(0.7); 7.994(1.1): 7.947(5.2); 7.926(12.3); 7.896(10.9); 7.875(4.8); |
| | | | 7.840(0.4); 7.820(0.5); 7.576(8.3); 7.555(7.8); 7.538(0.5); 7.528(0.9); 4.670(6.1); |
| | | | 4.654(6.2); 4.614(0.7); 4.598(0.7); 3.682(0.5); 3.625(0.5); 3.329(400.9); 2.676 |
| | | | (2.5); 2.672(3.4); 2.668(2.5); 2.525(9.5); 2.507(365.7); 2.503(478.4); 2.498(356.2); |
| | | | 2.334(2.3); 2.329(3.2); 2.325(2.3); 2.061(0.6); 2.045(1.7); 2.038(1.4); 2.031(2.5); |
| | | | 2.015(1.9); 2.000(0.7); 1.234(0.8); 1.148(0.3); 1.070(0.4); 1.045(1.1); |
| | | | 1.037(1.5); 1.026(1.2); 1.020(0.6); 0.994(0.4); 0.985(0.4); 0.924(1.2); 0.913(1.3); |
| | | | 0.904(1.4); 0.895(1.2); 0.869(14.0); 0.858(7.7); 0.850(6.6); 0.826(0.5); 0.146(2.4); |
| | | | 0.008(17.9); 0.000(478.3); −0.007(19.7); −0.150(2.4) |
| I-094 | 3.84 | 3.96 | Example I-094: ¹H-NMR(400.0 MHz, d₆-DMSO): δ = |
| | | | 10.992(7.3); 9.705(12.2); 9.691(1.9); 9.436(0.6); 8.898(2.0); 8.882(4.2); 8.866 |
| | | | (2.0); 8.535(8.3); 8.394(1.0); 8.316(1.8); 8.166(1.0); 8.078(1.1); 8.049(9.7); |
| | | | 8.029(10.6); 8.013(1.8); 8.001(8.5); 7.998(9.3); 7.924(9.2); 7.902(10.6); 7.840(0.5); |
| | | | 7.820(0.6); 7.574(9.8); 7.552(15.2); 7.528(8.2); 4.667(7.6); 4.652(7.7); 4.611(0.9); |
| | | | 4.596(1.0); 3.704(0.4); 3.624(0.4); 3.418(0.3); 3.327(1009.6); 3.260(0.6); |
| | | | 3.219(0.3); 3.175(0.4); 3.162(0.4); 2.828(0.4); 2.671(7.7); 2.627(0.4); 2.614(0.4); |
| | | | 2.502(1177.9); 2.365(0.8); 2.329(7.9); 2.286(0.4); 2.284(0.4); 2.255(0.3); |
| | | | 2.232(0.3); 2.214(0.3); 2.193(0.3); 2.075(2.5); 2.056(0.9); 2.040(2.2); 2.026 |
| | | | (3.3); 2.012(2.4); 1.994(0.9); 1.984(0.3); 1.250(0.4); 1.234(1.3); 1.147(0.8); |
| | | | 1.130(0.3); 1.110(0.4); 1.043(1.5); 1.034(2.0); 0.993(0.5); 0.981(0.5); 0.956(0.5); |
| | | | 0.923(1.6); 0.911(1.7); 0.902(1.9); 0.893(1.6); 0.865(16.0); 0.856(10.4); |
| | | | 0.846(8.2); 0.146(4.8); 0.000(962.9); −0.099(0.4); −0.150(5.0) |
| I-095 | 4.61 | 4.79 | Example I-095: ¹H-NMR(400.0 MHz, d₆-DMSO): δ = |
| | | | 10.959(5.9); 9.396(0.3); 9.379(0.7); 9.362(0.3); 8.838(1.6); 8.823(3.5); 8.807(1.8); |
| | | | 8.524(7.2); 8.520(7.6); 8.376(1.5); 8.372(1.6); 8.317(1.7); 8.158(1.6); 8.154(1.6); |
| | | | 8.069(1.8); 8.047(2.0); 8.040(0.5); 7.977(8.1); 7.973(8.3); 7.914(8.9); |
| | | | 7.897(3.4); 7.892(10.2); 7.546(6.9); 7.526(6.0); 7.428(6.3); 7.424(6.7); 7.399(5.9); |
| | | | 7.378(8.0); 7.361(1.6); 7.341(1.8); 7.233(3.9); 7.230(4.0); 7.213(3.4); 7.209(4.0); |
| | | | 7.185(0.9); 4.575(6.9); 4.560(7.0); 4.522(1.4); 4.506(1.3); 3.728(0.5); |
| | | | 3.710(0.6); 3.702(0.5); 3.682(0.9); 3.617(0.3); 3.600(0.4); 3.577(0.4); 3.564(0.4); |
| | | | 3.327(487.5); 2.846(0.6); 2.829(0.5); 2.810(0.3); 2.693(0.3); 2.676(3.8); |
| | | | 2.671(5.3); 2.667(4.0); 2.525(14.5); 2.511(292.9); 2.507(586.2); 2.502(777.6); |
| | | | 2.498(590.9); 2.406(1.0); 2.401(0.9); 2.359(1.2); 2.333(4.1); 2.329(5.6); |
| | | | 2.325(4.4); 2.258(0.4); 2.223(0.5); 2.207(0.5); 2.191(0.5); 2.177(0.5); 2.156(0.5); |
| | | | 2.131(0.4); 2.120(0.4); 2.103(0.4); 2.057(0.8); 2.041(1.9); 2.034(1.8); 2.027(3.1); |
| | | | 2.015(2.4); 1.996(1.4); 1.985(0.6); 1.233(1.8); 1.147(0.6); 1.107(0.8); 1.090(0.7); |
| | | | 1.069(0.7); 1.060(0.7); 1.046(1.1); 1.036(2.0); 1.028(2.7); 1.017(2.2); 1.010(1.3); |
| | | | 0.983(0.6); 0.964(0.7); 0.953(0.7); 0.948(0.8); 0.934(0.9); 0.919(1.4); 0.911(2.3); |
| | | | 0.901(2.3); 0.892(2.7); 0.882(2.7); 0.866(16.0); 0.854(8.8); 0.848(7.9); 0.781(0.4); |
| | | | 0.146(3.8); 0.008(33.7); 0.000(878.5); −0.008(44.0); −0.072(0.4); −0.150(4.0) |
| I-096 | 3.47 | | Example I-096: ¹H-NMR(400.0 MHz, d₆-DMSO): δ = |
| | | | 11.081(6.3); 8.999(1.7); 8.983(3.7); 8.967(1.8); 8.623(7.7); 8.619(8.0); 8.318(0.5); |
| | | | 8.302(5.8); 8.281(10.7); 8.231(12.3); 8.209(6.8); 8.071(8.4); 8.067(8.6); |
| | | | 7.843(9.2); 7.822(10.8); 7.562(9.2); 7.541(8.0); 5.758(15.8); 4.666(6.6); |
| | | | 4.651(6.7); 3.332(145.4); 2.677(0.7); 2.673(1.0); 2.668(0.8); 2.526(2.7); 2.513(56.5); |
| | | | 2.508(114.2); 2.504(151.2); 2.499(113.5); 2.495(58.3); 2.335(0.8); 2.330(1.0); |
| | | | 2.326(0.8); 2.086(0.6); 2.071(1.9); 2.055(2.4); 2.040(2.1); 2.025(0.7); 1.233(0.7); |
| | | | 0.956(0.4); 0.938(0.8); 0.919(0.5); 0.882(16.0); 0.867(10.5); 0.812(0.3); 0.146(0.9); |
| | | | 0.008(7.3); 0.000(194.7); −0.008(9.0); −0.150(0.9) |
| I-097 | 3.11 | 3.24 | Example I-097: ¹H-NMR(400.0 MHz, d₆-DMSO): δ = |
| | | | 11.951(0.5); 11.066(6.3); 10.056(0.4); 10.042(0.8); 10.027(0.5); 9.088(1.7); |
| | | | 9.073(3.6); 9.057(1.7); 9.044(0.6); 9.005(1.2); 8.790(1.2); 8.773(4.9); 8.636(0.5); |
| | | | 8.631(0.6); 8.614(7.8); 8.610(7.9); 8.590(3.6); 8.580(0.7); 8.573(0.8); 8.476(0.8); |
| | | | 8.460(0.8); 8.394(0.9); 8.372(1.3); 8.355(0.6); 8.336(0.4); 8.317(5.3); 8.300(6.3); |
| | | | 8.279(10.7); 8.252(0.6); 8.227(12.1); 8.205(6.8); 8.176(1.5); 8.151(0.5); |
| | | | 8.059(8.1); 8.055(8.6); 8.044(3.2); 8.024(9.3); 8.010(5.1); 8.005(5.8); 7.990 |
| | | | (1.8); 7.985(2.2); 7.874(0.6); 7.855(0.6); 7.717(0.3); 7.688(0.8); 7.672(1.0); |
| | | | 7.497(0.5); 7.470(0.5); 6.810(0.3); 6.792(0.5); 6.775(0.3); 5.923(0.4); 5.757(2.6); |
| | | | 4.789(1.4); 4.774(1.4); 4.695(6.7); 4.679(6.7); 4.041(0.6); 4.015(0.7); 3.931(0.3); |
| | | | 3.649(0.3); 3.626(0.3); 3.573(0.4); 3.553(0.4); 3.546(0.4); 3.508(0.6); 3.486(0.5); |
| | | | 3.469(0.6); 3.383(1.5); 3.376(1.8); 3.327(2799.1); 3.275(0.8); 3.257(0.5); |
| | | | 3.230(0.4); 3.225(0.3); 3.146(0.4); 3.141(0.4); 3.128(0.7); 3.060(0.3); 2.924(0.3); |
| | | | 2.883(0.3); 2.857(0.3); 2.826(0.4); 2.814(0.4); 2.805(0.5); 2.796(0.4); 2.680(5.4); |
| | | | 2.676(11.4); 2.671(16.0); 2.666(12.2); 2.635(1.1); 2.612(1.2); 2.524 |
| | | | (39.6); 2.520(59.6); 2.511(797.0); 2.506(1646.0); 2.502(2230.8); 2.497(1692.8); |
| | | | 2.493(866.6); 2.462(5.3); 2.333(10.6); 2.329(15.1); 2.324(11.2); 2.094(0.5); |
| | | | 2.079(1.8); 2.064(2.4); 2.049(2.1); 2.032(0.6); 1.350(0.8); 1.335(0.4); 1.248(0.7); |
| | | | 1.235(2.8); 1.194(0.4); 1.177(0.4); 1.147(0.4); 1.044(0.4); 1.029(0.4); 0.881(15.4); |
| | | | 0.865(12.0); 0.008(0.5); 0.000(16.4) |
| I-098 | 4.11 | 4.21 | Example I-098: ¹H-NMR(400.0 MHz, d₆-DMSO): δ = |
| | | | 20.006(0.7); 10.962(2.5); 8.955(0.8); 8.937(1.6); 8.515(2.8); 8.511(3.1); 8.317 |
| | | | (5.8); 7.982(3.3); 7.979(3.4); 7.840(3.6); 7.819(6.2); 7.720(1.2); 7.693(1.3); |
| | | | 7.555(3.6); 7.534(3.3); 7.488(1.3); 7.468(1.4); 5.757(1.3); 4.655(2.7); 4.640(2.6); |
| | | | 3.327(1609.4); 2.675(11.6); 2.671(16.0); 2.667(12.1); 2.524(40.0); 2.511(854.7); |
| | | | 2.506(1743.3); 2.502(2317.4); 2.497(1727.5); 2.493(877.6); 2.378(10.8); |
| | | | 2.333(11.1); 2.329(15.5); 2.324(11.5); 2.037(1.0); 1.351(1.9); 1.336(1.2); |
| | | | 1.235(2.8); 0.959(1.3); 0.868(5.9); 0.857(3.4); 0.851(3.0); 0.000(25.7) |
| I-099 | 3.95 | 4.03 | Example I-099: ¹H-NMR(400.0 MHz, d₆-DMSO): δ = |
| | | | 10.975(3.5); 8.950(1.0); 8.934(2.0); 8.919(1.0); 8.201(4.3); 8.197(4.5); 7.840(5.0); |
| | | | 7.819(6.0); 7.670(4.7); 7.667(4.8); 7.555(5.2); 7.535(4.6); 7.424(2.7); 7.403(4.1); |
| | | | 7.386(2.8); 7.326(1.6); 7.3O5(1.1); 5.757(5.2); 4.646(3.9); 4.631(3.9); |
| | | | 3.331(171.2); 2.676(0.7); 2.672(0.9); 2.668(0.7); 2.507(104.9); 2.503(138.1); |
| | | | 2.499(104.3); 2.334(0.7); 2.330(0.9); 2.325(0.7); 2.283(16.0); 2.041(1.1); |
| | | | 2.026(1.4); 2.011(1.1); 1.995(0.4); 1.351(0.4); 1.234(0.7); 0.844(6.1); 0.829(9.2); |
| | | | 0.000(0.5) |
| I-100 | 3.75 | 3.86 | Example I-100: ¹H-NMR(400.0 MHz, d₆-DMSO): δ = |
| | | | 11.021(2.5); 8.738(0.9); 8.723(1.7); 8.708(0.8); 8.547(3.2); 8.543(3.0); 8.010(3.6); |
| | | | 8.006(3.3); 7.929(3.8); 7.924(1.6); 7.912(1.9); 7.907(4.2); 7.900(0.6); 7.786(3.8); |
| | | | 7.765(4.2); 7.728(3.4); 7.723(3.4); 7.713(0.9); 7.694(0.5); 7.687(0.3); |
| | | | 7.556(2.9); 7.536(2.5); 7.399(3.8); 7.378(3.4); 7.349(0.4); 6.679(3.6); 6.673(3.3); |
| | | | 6.656(0.4); 6.650(0.3); 5.765(1.4); 4.591(2.9); 4.576(2.7); 3.926(0.3); 3.890 |
| | | | (2.9); 3.880(16.0); 3.872(2.2); 3.398(9.9); 2.683(0.7); 2.678(0.8); 2.674(0.6); |
| | | | 2.518(70.6); 2.514(105.2); 2.509(120.1); 2.505(83.1); 2.500(39.9); 2.340(0.7); |
| | | | 2.336(0.9); 2.331(0.6); 2.041(0.5); 2.029(0.8); 2.024(1.0); 2.011(1.3); 1.998(0.8); |
| | | | 1.980(0.4); 1.358(0.3); 1.339(0.4); 1.289(1.0); 1.264(0.8); 1.241(1.0); 1.044 |
| | | | (0.5); 1.037(0.5); 1.001(0.3); 0.920(0.8); 0.912(0.9); 0.901(1.4); 0.882(2.7); |
| | | | 0.865(6.2); 0.856(4.4); 0.848(3.0); 0.845(2.9) |
| I-101 | 3.97 | 4.08 | Example I-101: ¹H-NMR(400.0 MHz, d₆-DMSO): δ = |
| | | | 10.986(2.6); 8.908(0.7); 8.893(1.5); 8.877(0.7); 8.535(3.1); 8.531(3.2); 7.993(3.5); |
| | | | 7.989(3.6); 7.965(3.9); 7.944(4.4); 7.928(0.6); 7.920(4.0); 7.915(1.5); 7.903(1.5); |
| | | | 7.898(4.5); 7.891(0.6); 7.548(2.8); 7.528(2.5); 7.507(3.8); 7.486(3.5); |
| | | | 4.658(2.7); 4.642(2.7); 3.847(16.0); 3.838(1.3); 3.332(165.0); 2.677(0.6); |
| | | | 2.672(0.8); 2.668(0.6); 2.525(1.7); 2.512(43.9); 2.507(89.8); 2.503(118.8); |
| | | | 2.498(87.3); 2.494(43.4); 2.334(0.6); 2.330(0.8); 2.325(0.6); 2.043(0.6); 2.037(0.5); |
| | | | 2.029(1.1); 2.012(0.7); 1.233(0.8); 1.140(0.4); 1.123(0.4); 0.867(5.7); 0.857(3.3); |
| | | | 0.847(2.8); 0.008(0.6); 0.000(18.2); −0.009(0.6) |
| I-102 | 4.16 | 4.29 | Example I-102: ¹H-NMR(400.0 MHz, d₆-DMSO): δ = |
| | | | 11.007(6.2); 8.976(1.9); 8.962(3.7); 8.947(2.0); 8.539(7.4); 8.319(0.4); 8.157(2.9); |
| | | | 8.153(3.4); 8.128(3.9); 8.116(3.8); 8.094(3.8); 8.055(0.5); 7.980(7.8); 7.918(8.0); |
| | | | 7.896(9.0); 7.691(2.6); 7.671(4.3); 7.651(2.4); 7.547(7.5); 7.526(6.8); |
| | | | 7.210(0.3); 5.758(0.8); 4.719(6.9); 4.704(6.9); 3.355(58.1); 2.672(1.7); |
| | | | 2.503(252.1); 2.370(0.5); 2.329(1.9); 2.179(0.3); 2.162(0.3); 2.087(0.8); 2.071(2.0); |
| | | | 2.056(2.7); 2.042(2.2); 2.026(1.0); 1.523(0.4); 1.491(0.3); 1.475(0.3); 1.234(1.8); |
| | | | 1.042(0.6); 0.992(0.4); 0.873(16.0); 0.862(10.7); 0.146(3.6); 0.000(609.1); −0.150(3.7) |
| I-103 | 3.17 | 3.28 | Example I-103: ¹H-NMR(400.0 MHz, d₆-DMSO): δ = |
| | | | 11.073(5.3); 9.065(0.4); 9.048(0.4); 8.710(2.1); 8.613(0.3); 8.599(7.1); 8.595(7.5); |
| | | | 8.353(3.3); 8.342(3.6); 8.318(2.9); 8.296(6.0); 8.275(9.7); 8.219(11.1); 8.198(6.4); |
| | | | 8.177(0.4); 8.158(0.5); 8.125(0.6); 8.115(0.7); 8.104(0.9); 8.062(0.4); |
| | | | 8.032(0.4); 8.007(7.7); 8.003(7.8); 7.917(0.4); 7.896(0.4); 7.827(0.4); 7.790(0.3); |
| | | | 7.691(0.3); 7.670(1.2); 7.654(1.6); 7.591(0.3); 7.542(0.4); 7.431(0.4); 7.304(0.4); |
| | | | 7.298(0.4); 5.757(1.9); 3.735(0.4); 3.702(0.4); 3.681(0.6); 3.650(2.1); |
| | | | 3.639(1.0); 3.626(0.5); 3.619(0.6); 3.610(0.6); 3.574(0.7); 3.358(371.1); 3.168 |
| | | | (1.0); 3.099(0.5); 3.081(0.5); 3.060(0.4); 3.021(0.4); 2.951(0.5); 2.941(1.1); |
| | | | 2.932(1.5); 2.923(2.2); 2.913(2.3); 2.904(1.5); 2.894(1.2); 2.884(0.6); 2.762(0.3); |
| | | | 2.676(6.2); 2.671(8.6); 2.667(6.5); 2.576(0.4); 2.525(21.4); 2.511(516.2); |
| | | | 2.507(1032.4); 2.502(1350.8); 2.498(1008.2); 2.494(517.3); 2.417(1.3); 2.378(0.8); |
| | | | 2.333(6.6); 2.329(9.0); 2.324(6.8); 2.275(0.5); 2.225(0.4); 2.209(0.5); 2.185 |
| | | | (0.5); 2.179(0.5); 2.169(0.5); 2.071(0.8); 2.055(2.1); 2.040(2.6); 2.024(2.3); |
| | | | 2.008(0.8); 1.500(0.4); 1.292(0.4); 1.256(0.9); 1.235(2.9); 1.200(0.5); 1.196(0.4); |
| | | | 1.177(0.6); 1.159(0.4); 1.069(0.5); 1.046(0.6); 1.017(0.7); 0.991(0.9); 0.972(0.8); |
| | | | 0.955(0.9); 0.951(0.8); 0.942(0.8); 0.907(1.4); 0.879(12.8); 0.864(16.0); |
| | | | 0.809(1.8); 0.796(4.3); 0.791(5.9); 0.779(5.6); 0.773(4.8); 0.761(2.1); |
| | | | 0.741(0.6); 0.723(0.7); 0.712(0.8); 0.685(0.5); 0.675(0.4); 0.645(2.0); 0.634(5.4); |
| | | | 0.628(5.2); 0.624(4.9); 0.619(4.5); 0.606(1.4); 0.146(21.0); 0.008(194.6); |
| | | | 0.000(4187.4); −0.009(245.7); −0.029(6.1); −0.030(6.0); −0.031 |
| | | | (6.0); −0.039(4.0); −0.043(3.2); −0.045(3.1); −0.058(2.4); −0.078 |
| | | | (1.6); −0.087(1.4); −0.096(1.0); −0.103(1.0); −0.131(0.8); −0.150 |
| | | | (21.5); −0.177(0.6); −0.190(0.5); −0.194(0.6); −0.199(0.5); −0.223 |
| | | | (0.5); −0.253(0.5); −0.256(0.4); −0.275(0.3); − 0.294(0.4) |
| I-104 | 3.87 | 3.99 | Example I-104: ¹H-NMR(400.0 MHz, d₆-DMSO): δ = |
| | | | 10.604(2.7); 8.817(0.8); 8.801(1.6); 8.786(0.8); 8.586(3.5); 8.582(3.5); 8.440(5.8); |
| | | | 8.318(0.6); 8.004(3.8); 8.000(3.8); 7.939(4.2); 7.917(4.7); 7.733(3.9); 7.713(4.7); |
| | | | 7.676(6.1); 7.666(0.3); 7.557(3.3); 7.537(3.0); 7.478(4.1); 7.457(3.5); |
| | | | 4.607(3.0); 4.592(3.0); 3.622(1.1); 3.579(0.5); 3.351(89.0); 2.805(0.4); 2.789(1.0); |
| | | | 2.771(1.4); 2.754(1.1); 2.737(0.5); 2.676(1.5); 2.672(2.1); 2.667(1.6); 2.524(6.3); |
| | | | 2.507(239.2); 2.502(311.3); 2.498(230.0); 2.333(1.5); 2.329(2.0); 2.325(1.5); |
| | | | 1.521(0.9); 1.239(0.8); 1.176(0.4); 1.158(0.4); 1.141(1.1); 1.125(16.0); |
| | | | 1.108(15.2); 1.087(0.5); 1.060(1.6); 1.048(0.6); 1.042(0.9); 1.025(0.7); 0.008 |
| | | | (2.1); 0.000(63.8); −0.008(2.8) |
| I-105 | 3.71 | 3.86 | Example I-105: ¹H-NMR(400.0 MHz, d₆-DMSO): δ = |
| | | | 10.999(5.8); 8.636(1.3); 8.627(1.4); 8.540(7.4); 8.537(7.4); 8.327(3.6); 8.317(4.3); |
| | | | 7.946(8.2); 7.942(8.1); 7.930(4.9); 7.909(14.4); 7.888(12.6); 7.867(4.3); |
| | | | 7.510(0.8); 7.492(1.1); 7.477(0.9); 3.602(0.4); 3.574(0.5); 3.513(0.6); 3.498(0.7); |
| | | | 3.482(0.8); 3.341(617.4); 3.242(0.7); 3.208(0.4); 3.168(0.5); 2.943(0.4); |
| | | | 2.932(1.1); 2.923(1.5); 2.914(2.3); 2.904(2.4); 2.896(1.6); 2.886(1.2); 2.875(0.5); |
| | | | 2.776(0.4); 2.762(0.5); 2.754(0.4); 2.742(0.4); 2.676(3.5); 2.672(4.6); 2.620(0.4); |
| | | | 2.507(531.6); 2.503(677.7); 2.499(508.7); 2.439(0.7); 2.334(3.2); 2.329(4.3); |
| | | | 2.325(3.2); 2.060(0.6); 2.044(2.1); 2.029(2.8); 2.014(2.3); 2.005(0.5); 1.999(0.7); |
| | | | 1.909(0.7); 1.234(2.2); 0.872(16.0); 0.856(15.0); 0.804(1.6); 0.791(4.5); |
| | | | 0.786(5.9); 0.774(5.6); 0.768(4.8); 0.757(1.9); 0.736(0.3); 0.717(0.4); 0.669(0.3); |
| | | | 0.639(1.9); 0.628(5.9); 0.622(5.6); 0.613(5.0); 0.601(1.5); 0.146(1.1); 0.008(9.6); |
| | | | 0.000(245.7); −0.150(1.3) |
| I-106 | 3.33 | 3.42 | Example I-106: ¹H-NMR(400.0 MHz, d₆-DMSO): δ = |
| | | | 11.055(5.9); 9.028(2.0); 9.012(8.2); 9.008(6.3); 8.998(2.1); 8.978(0.3); 8.556(7.9); |
| | | | 8.552(7.7); 8.517(0.5); 8.513(0.5); 8.496(1.2); 8.389(0.5); 8.385(0.5); 8.317(2.3); |
| | | | 8.312(4.4); 8.307(4.4); 8.292(4.5); 8.286(4.6); 8.188(0.5); 8.184(0.5); |
| | | | 8.087(0.5); 8.080(0.7); 8.064(0.6); 8.058(0.6); 8.018(0.3); 7.993(8.9); 7.989(8.7); |
| | | | 7.945(0.6); 7.941(0.7); 7.921(9.4); 7.916(3.5); 7.903(3.7); 7.898(10.6); 7.891(1.3); |
| | | | 7.886(0.8); 7.608(5.4); 7.588(5.0); 7.549(7.4); 7.528(6.5); 4.809(0.6); 4.780(7.4); |
| | | | 4.765(7.4); 4.720(0.4); 4.703(0.4); 3.918(0.4); 3.890(0.9); 3.734(1.0); |
| | | | 3.657(0.8); 3.639(0.4); 3.626(0.4); 3.614(0.4); 3.610(0.4); 3.543(0.4); 3.521(0.4); |
| | | | 3.504(0.4); 3.351(499.4); 3.168(0.4); 2.676(3.1); 2.672(4.2); 2.667(3.2); |
| | | | 2.619(0.3); 2.576(0.6); 2.525(11.0); 2.520(17.5); 2.511(228.5); 2.507(465.0); |
| | | | 2.503(612.2); 2.498(449.5); 2.494(221.5); 2.334(2.8); 2.329(3.9); 2.325(2.9); |
| | | | 2.102(0.5); 2.085(1.5); 2.071(2.5); 2.055(1.8); 2.040(0.9); 2.030(0.4); 1.234(1.0); |
| | | | 1.190(0.6); 1.172(1.2); 1.154(0.6); 1.067(0.4); 1.056(0.7); 1.048(0.9); 1.037(0.7); |
| | | | 1.001(0.5); 0.993(0.6); 0.977(0.4); 0.968(0.4); 0.949(0.6); 0.941(0.9); 0.931(0.9); |
| | | | 0.919(1.4); 0.902(1.6); 0.881(16.0); 0.870(9.1); 0.862(8.5); 0.785(0.4); |
| | | | 0.769(0.4); 0.757(0.4); 0.008(2.0); 0.000(71.1); −0.009(2.8) |
| I-107 | 3.80 | 3.86 | Example I-107: ¹H-NMR(400.0 MHz, d₆-DMSO): δ = |
| | | | 11.001(2.4); 8.839(0.6); 8.824(1.4); 8.808(0.7); 8.539(2.8); 8.536(2.9); 8.004(3.0); |
| | | | 8.001(3.1); 7.924(3.4); 7.902(4.0); 7.551(2.7); 7.539(2.4); 7.530(2.6); 7.519(5.7); |
| | | | 7.495(4.9); 7.474(2.1); 7.463(3.3); 7.459(3.3); 7.450(0.6); 7.446(0.5); |
| | | | 6.389(3.1); 6.385(3.2); 4.638(2.5); 4.623(2.6); 3.848(16.0); 3.833(1.1); 3.821(0.7); |
| | | | 3.336(21.7); 3.170(4.5); 2.672(0.4); 2.526(0.8); 2.508(45.6); 2.503(60.1); |
| | | | 2.499(44.6); 2.330(0.4); 2.076(3.6); 2.035(0.6); 2.022(1.0); 2.005(0.7); 1.233(0.5); |
| | | | 0.867(5.1); 0.857(3.1); 0.847(2.6); 0.000(1.2) |
| I-108 | 3.64 | 3.77 | Example I-108: ¹H-NMR(400.0 MHz, d₆-DMSO): δ = |
| | | | 11.097(1.7); 8.571(4.1); 8.567(2.9); 7.974(2.5); 7.970(2.4); 7.936(2.6); 7.914(3.0); |
| | | | 7.551(2.0); 7.530(1.8); 3.331(101.4); 2.672(0.7); 2.667(0.5); 2.507(75.7); |
| | | | 2.503(97.2); 2.498(72.7); 2.334(0.5); 2.330(0.6); 2.325(0.5); 2.068(0.6); 2.053 |
| | | | (0.8); 2.038(0.6); 1.769(16.0); 1.747(0.5); 1.269(0.8); 1.234(0.8); 0.880(4.9); |
| | | | 0.864(3.6); 0.146(0.4); 0.008(3.3); 0.000(79.5); −0.008(4.2); −0.150(0.4) |
| I-109 | 3.68 | 3.78 | Example I-109: ¹H-NMR(400.0 MHz, d₆-DMSO): δ = |
| | | | 11.027(5.9); 9.188(6.9); 8.552(7.1); 8.549(7.3); 8.317(0.5); 7.946(7.9); 7.943(8.2); |
| | | | 7.933(8.3); 7.911(9.2); 7.552(6.8); 7.531(6.2); 5.758(1.3); 3.333(307.8); |
| | | | 2.769(1.3); 2.763(1.2); 2.756(1.8); 2.748(2.4); 2.735(3.5); 2.723(3.1); 2.716(2.5); |
| | | | 2.703(2.1); 2.672(1.7); 2.621(0.4); 2.562(1.9); 2.538(4.4); 2.503(258.0); |
| | | | 2.429(0.5); 2.420(0.5); 2.407(0.5); 2.400(0.4); 2.379(0.3); 2.330(1.7); 2.138(0.8); |
| | | | 2.131(0.7); 2.117(1.2); 2.110(2.0); 2.088(4.6); 2.071(4.6); 2.060(3.8); 2.047(2.5); |
| | | | 2.023(1.0); 1.995(0.4); 1.351(0.8); 1.336(0.4); 1.257(0.4); 1.249(0.6); 1.234(1.7); |
| | | | 0.878(16.0); 0.864(10.3); 0.146(0.6); 0.000(127.1); −0.150(0.7) |
| I-110 | 4.19 | 4.33 | Example I-110: ¹H-NMR(400.0 MHz, d₆-DMSO): δ = |
| | | | 10.594(2.9); 9.972(0.5); 8.957(0.8); 8.941(1.7); 8.926(0.8); 8.589(3.5); 8.585(3.7); |
| | | | 8.487(2.4); 8.085(0.7); 8.063(0.8); 8.043(0.4); 8.025(1.5); 8.012(1.5); 8.002(1.5); |
| | | | 7.989(1.5); 7.979(4.0); 7.975(4.1); 7.941(0.5); 7.934(4.4); 7.929(1.6); |
| | | | 7.917(1.5); 7.912(5.2); 7.904(0.7); 7.555(3.3); 7.535(4.3); 7.525(1.7); 7.511(1.5); |
| | | | 7.498(0.4); 5.758(2.5); 4.735(0.8); 4.720(0.8); 4.659(2.8); 4.643(2.8); 3.332 |
| | | | (91.6); 2.892(0.4); 2.844(0.4); 2.827(1.0); 2.810(1.4); 2.793(1.1); 2.776(0.4); |
| | | | 2.732(0.3); 2.677(0.5); 2.672(0.7); 2.668(0.5); 2.525(1.6); 2.512(38.0); 2.508(78.8); |
| | | | 2.503(105.8); 2.499(78.4); 2.494(38.9); 2.334(0.5); 2.330(0.7); 2.326(0.5); |
| | | | 1.232(0.4); 1.140(16.0); 1.123(15.8); 0.937(0.5); 0.146(0.8); 0.008(5.8); |
| | | | 0.000(167.8); −0.009(6.3); −0.150(0.8) |
| I-111 | 4.02 | 4.18 | Example I-112: ¹H-NMR(400.0 MHz, d₆-DMSO): δ = 0.88(m, 4H), |
| | | | 2.07(m, 1H), 4.66(d, 2H), 7.42(d, 2H), 7.55(d, 2H), 7.92(d, 2H), 7.98(s, |
| | | | 1H), 8.53(s, 1H), 9.05(m, 1H), 11.97(s, 1H) |
| I-112 | 3.37 | 3.41 | Example I-112: ¹H-NMR(400.0 MHz, d₆-DMSO): δ = |
| | | | 10.994(1.5); 9.342(3.3); 9.327(0.3); 8.931(0.4); 8.915(0.9); 8.899(0.4); 8.538(1.8); |
| | | | 8.534(1.8); 8.317(0.5); 8.037(2.2); 8.016(2.5); 8.001(2.0); 7.997(2.0); 7.924(2.2); |
| | | | 7.902(2.5); 7.595(2.2); 7.575(2.1); 7.550(1.8); 7.529(1.5); 4.676(1.6); |
| | | | 4.660(1.6); 3.333(210.6); 2.676(1.2); 2.671(1.6); 2.667(1.2); 2.507(186.1); |
| | | | 2.502(245.8); 2.498(186.0); 2.333(1.1); 2.329(1.6); 2.325(1.2); 2.045(0.4); |
| | | | 2.032(0.6); 2.016(0.5); 1.233(16.0); 0.904(0.3); 0.895(0.3); 0.866(3.9); 0.854(3.3); |
| | | | 0.837(1.2); 0.326(0.3); 0.146(1.1); 0.008(7.8); |
| | | | 0.000(235.7); −0.008(14.5); −0.150(1.2) |
| I-113 | 4.06 | 4.18 | Example I-113: ¹H-NMR(400.0 MHz, d₆-DMSO): δ = |
| | | | 10.994(0.5); 8.518(0.6); 8.515(0.6); 7.944(0.7); 7.940(0.6); 7.906(0.7); 7.884(0.8); |
| | | | 7.543(0.5); 7.522(0.5); 5.758(16.0); 3.366(0.5); 3.350(0.5); 3.330(7.8); 2.508(14.3); |
| | | | 2.503(18.3); 2.499(13.4); 2.087(0.8); 1.544(0.4); 1.369(0.4); 1.350(0.4); |
| | | | 0.943(0.8); 0.925(1.6); 0.906(0.7); 0.877(1.3); 0.863(0.8); 0.008(0.5); |
| | | | 0.000(12.1); −0.008(0.5) |
| I-114 | 3.23 | 3.33 | Example I-114: ¹H-NMR(400.0 MHz, d₆-DMSO): δ = |
| | | | 10.956(2.4); 8.875(0.7); 8.860(1.4); 8.845(0.7); 8.634(2.7); 8.629(2.7); 8.519(2.9); |
| | | | 8.515(2.8); 8.318(0.5); 8.224(2.5); 8.219(2.4); 8.142(5.4); 7.985(3.1); 7.981(3.0); |
| | | | 7.913(3.4); 7.891(3.8); 7.544(2.6); 7.524(2.3); 4.716(2.8); 4.701(2.8); |
| | | | 4.057(16.0); 4.046(0.7); 3.338(74.1); 3.168(0.6); 2.676(1.0); 2.672(1.3); 2.667 |
| | | | (1.0); 2.525(3.7); 2.507(158.3); 2.503(202.9); 2.499(149.4); 2.334(1.0); 2.329(1.3); |
| | | | 2.325(1.0); 2.023(0.6); 2.009(0.9); 1.995(0.6); 1.234(0.4); 0.855(5.3); 0.845(3.1); |
| | | | 0.835(2.6); 0.146(0.6); 0.008(4.5); 0.000(120.8); −0.150(0.6) |
| I-115 | 3.40 | 3.46 | Example I-115: ¹H-NMR(400.0 MHz, d₆-DMSO): δ = |
| | | | 11.021(5.7); 9.065(3.5); 9.046(3.5); 8.547(7.4); 8.543(7.5); 8.318(1.0); 7.970(8.2); |
| | | | 7.966(8.2); 7.933(8.8); 7.928(3.3); 7.916(3.3); 7.911(10.0); 7.550(6.3); 7.530(5.8); |
| | | | 5.758(6.2); 5.108(0.6); 5.091(2.5); 5.072(3.6); 5.054(2.5); 5.036(0.6); |
| | | | 3.330(176.7); 2.676(1.6); 2.672(2.2); 2.667(1.7); 2.525(6.0); 2.511(129.1); |
| | | | 2.507(260.5); 2.503(340.9); 2.498(251.2); 2.334(1.6); 2.329(2.2); 2.325(1.6); |
| | | | 2.112(0.5); 2.096(1.7); 2.087(5.2); 2.081(2.5); 2.065(1.9); 2.050(0.7); 1.602(14.9); |
| | | | 1.584(14.9); 1.433(0.6); 1.415(0.6); 1.398(0.4); 1.234(1.1); 0.877(16.0); 0.863(9.0); |
| | | | 0.859(8.8); 0.720(0.4); 0.146(0.8); 0.008(6.6); 0.000(188.5); −0.008(8.6); −0.150(0.9) |
| I-116 | 3.30 | 2.88 | Example I-116: ¹H-NMR(400.0 MHz, d₆-DMSO): δ = |
| | | | 11.063(5.4); 8.920(1.6); 8.905(3.4); 8.890(1.7); 8.580(2.8); 8.579(3.0); 8.576(2.7); |
| | | | 8.563(8.7); 8.559(7.6); 8.009(8.3); 8.005(8.5); 7.930(0.9); 7.923(8.8); 7.918(3.3); |
| | | | 7.906(3.1); 7.901(10.2); 7.894(1.4); 7.822(1.9); 7.818(1.9); 7.803(3.7); |
| | | | 7.799(3.8); 7.784(2.3); 7.780(2.3); 7.549(6.0); 7.529(5.4); 7.420(4.1); 7.401(3.7); |
| | | | 7.336(2.1); 7.334(2.1); 7.321(2.3); 7.317(2.3); 7.305(1.9); 7.303(1.9); 5.760(16.0); |
| | | | 4.701(7.4); 4.687(7.4); 4.039(0.4); 4.021(0.4); 3.338(37.9); 2.675(0.4); |
| | | | 2.528(1.0); 2.514(21.9); 2.510(45.5); 2.506(60.4); 2.501(44.9); 2.497(22.9); |
| | | | 2.332(0.4); 2.097(0.5); 2.088(2.4); 2.084(1.3); 2.080(1.4); 2.074(1.1); 2.066(2.2); |
| | | | 2.053(1.4); 2.050(1.4); 2.036(0.6); 1.991(1.8); 1.353(0.3); 1.229(0.6); 1.194(0.5); |
| | | | 1.177(1.0); 1.159(0.5); 0.881(11.9); 0.872(7.5); 0.861(6.0); 0.839(0.4); |
| | | | 0.008(1.4); 0.000(43.0); −0.009(2.0) |
| I-117 | 2.95 | 3.07 | Example I-117: ¹H-NMR(400.0 MHz, d₆-DMSO): δ = |
| | | | 11.028(5.5); 9.035(3.1); 9.017(3.1); 8.534(7.0); 8.531(7.3); 8.318(0.6); 7.924(8.0); |
| | | | 7.920(8.5); 7.912(8.8); 7.894(3.1); 7.890(9.8); 7.545(6.2); 7.524(5.7); 5.093(1.1); |
| | | | 5.075(2.4); 5.057(2.7); 5.040(1.5); 5.023(0.4); 4.846(5.4); 4.830(8.7); |
| | | | 4.812(5.1); 4.625(5.8); 4.609(10.7); 4.593(5.0); 3.331(72.7); 2.677(0.8); 2.672 |
| | | | (1.1); 2.668(0.9); 2.525(3.0); 2.508(129.0); 2.503(170.0); 2.499(127.4); 2.334(0.8); |
| | | | 2.330(1.1); 2.326(0.8); 2.094(0.5); 2.087(0.5); 2.079(1.8); 2.063(2.4); 2.048(2.1); |
| | | | 2.033(0.6); 1.233(0.5); 0.883(16.0); 0.868(11.3); 0.146(0.4); 0.008(3.1); |
| | | | 0.000(88.9); −0.150(0.4) |
| I-118 | 3.33 | 3.41 | Example I-118: ¹H-NMR(400.0 MHz, d₆-DMSO): δ = |
| | | | 11.014(4.3); 8.516(5.5); 8.317(3.4); 8.300(1.3); 7.945(5.8); 7.908(5.8); 7.887(6.4); |
| | | | 7.545(5.2); 7.524(4.6); 5.758(1.4); 3.569(16.0); 3.413(1.5); 3.395(3.8); 3.379(5.4); |
| | | | 3.360(6.2); 3.330(576.1); 3.287(1.2); 3.261(0.5); 2.732(0.5); 2.672(6.4); |
| | | | 2.503(948.8); 2.330(6.2); 2.061(1.5); 2.046(1.9); 2.031(1.6); 1.351(1.6); |
| | | | 1.336(0.8); 1.298(0.8); 1.234(4.9); 1.190(6.0); 1.172(11.9); 1.154(6.0); 1.120(0.4); |
| | | | 0.902(0.5); 0.874(12.4); 0.860(9.4); 0.146(1.6); 0.000(323.8); −0.149(1.8) |
| I-119 | 3.35 | 3.53 | Example I-119: ¹H-NMR(400.0 MHz, d₆-DMSO): δ = |
| | | | 11.064(1.7); 9.111(1.6); 9.109(1.6); 8.978(0.5); 8.962(1.0); 8.946(0.5); 8.682(1.6); |
| | | | 8.679(1.5); 8.615(2.4); 8.611(2.4); 8.045(2.6); 8.042(2.6); 7.841(2.6); 7.820(3.0); |
| | | | 7.561(2.6); 7.541(2.3); 4.658(1.9); 4.642(1.9); 3.569(16.0); 3.333(21.9); |
| | | | 2.526(0.6); 2.513(14.4); 2.508(29.3); 2.504(38.4); 2.499(28.4); 2.495(14.2); |
| | | | 2.057(0.6); 2.042(0.8); 2.026(0.6); 1.356(1.0); 0.862(3.8); 0.846(4.5); 0.008(2.1); |
| | | | 0.000(59.0); −0.008(2.8) |
| I-120 | 3.65 | 3.74 | Example I-120: ¹H-NMR(400.0 MHz, d₆-DMSO): δ = |
| | | | 11.059(6.1); 10.200(1.5); 9.051(1.7); 9.035(3.6); 9.020(1.7); 8.620(8.0); 8.616 |
| | | | (8.0); 8.600(0.5); 8.394(0.7); 8.374(1.2); 8.317(1.9); 8.313(2.6); 8.301(6.2); |
| | | | 8.279(10.9); 8.246(0.7); 8.230(12.6); 8.208(6.9); 8.166(0.4); 8.140(0.8); 8.119(0.6); |
| | | | 8.066(8.9); 8.062(8.6); 8.037(0.3); 7.934(3.2); 7.914(4.4); 7.897(3.7); 7.881(0.4); |
| | | | 7.873(0.4); 7.525(0.6); 7.521(0.6); 7.509(3.5); 7.484(3.5); 7.451(0.3); 7.425(0.3); |
| | | | 7.407(4.0); 7.387(3.7); 7.364(0.3); 7.064(0.4); 7.061(0.4); 7.031(0.4); |
| | | | 7.027(0.4); 6.391(0.4); 5.753(3.1); 4.673(6.6); 4.657(6.7); 4.616(0.4); 4.602(0.6); |
| | | | 4.587(0.4); 3.345(0.3); 3.315(211.2); 3.258(0.4); 2.675(3.1); 2.671(4.2); 2.666(3.0); |
| | | | 2.577(0.3); 2.524(11.0); 2.510(233.9); 2.506(468.1); 2.502(620.4); |
| | | | 2.497(458.8); 2.493(226.1); 2.453(1.1); 2.448(1.0); 2.389(0.4); 2.375(0.4); |
| | | | 2.369(0.3); 2.333(3.2); 2.328(4.3); 2.324(3.0); 2.220(0.4); 2.098(0.6); |
| | | | 2.083(1.8); 2.067(2.4); 2.052(2.0); 2.037(0.7); 1.347(0.4); 1.234(1.2); 1.224(2.2); |
| | | | 1.217(4.9); 0.884(16.0); 0.870(9.8); 0.146(2.5); 0.034(0.4); 0.008(20.0); |
| | | | 0.000(572.5); −0.009(22.0); −0.049(0.3); −0.054(0.3); −0.150(2.5) |
| I-121 | 3.83 | 3.94 | Example I-121: ¹H-NMR(400.0 MHz, d₆-DMSO): δ = |
| | | | 11.058(0.3); 10.993(5.9); 8.965(1.7); 8.949(3.5); 8.933(1.8); 8.513(7.1); 8.511 |
| | | | (7.0); 8.455(0.4); 8.314(0.8); 7.999(7.8); 7.996(8.1); 7.960(3.5); 7.838(8.0); |
| | | | 7.818(9.2); 7.785(0.6); 7.759(0.8); 7.737(3.2); 7.723(9.3); 7.704(0.9); 7.556(8.5); |
| | | | 7.536(7.5); 7.510(0.5); 7.485(0.4); 7.464(0.3); 6.871(0.3); 6.409(0.4); 5.754(5.4); |
| | | | 4.656(6.9); 4.641(6.9); 4.618(0.7); 4.602(0.5); 4.546(0.4); 4.531(0.4); 3.319(137.9); |
| | | | 2.671(1.8); 2.502(277.4); 2.329(1.8); 2.071(0.6); 2.056(1.8); 2.041(2.4); |
| | | | 2.026(2.0); 2.011(0.8); 1.300(0.3); 1.234(0.6); 0.872(16.0); 0.859(10.0); |
| | | | 0.696(0.4); 0.146(0.7); 0.000(153.6); −0.150(0.8) |
| I-122 | 3.99 | 4.18 | Example I-122: ¹H-NMR(400.0 MHz, d₆-DMSO): δ = |
| | | | 11.072(0.4); 11.023(6.1); 8.961(1.5); 8.945(3.3); 8.930(1.6); 8.460(0.5); 8.456 |
| | | | (0.4); 8.317(14.3); 8.310(8.3); 7.838(8.9); 7.818(10.7); 7.766(10.4); 7.763(9.1); |
| | | | 7.667(5.3); 7.645(7.7); 7.553(10.0); 7.533(8.6); 7.508(0.7); 4.647(6.2); 4.631 |
| | | | (6.5); 4.599(0.5); 3.328(508.1); 3.304(6.4); 3.283(0.4); 3.252(0.3); 2.675(6.2); |
| | | | 2.671(8.6); 2.667(6.6); 2.587(0.8); 2.574(0.8); 2.524(22.6); 2.506(984.3); |
| | | | 2.502(1318.4); 2.498(997.9); 2.333(6.1); 2.329(8.3); 2.324(6.4); 2.061(0.5); |
| | | | 2.047(1.7); 2.032(2.4); 2.015(1.8); 2.000(0.7); 1.398(2.1); 1.232(0.5); 1.148(1.0); |
| | | | 0.846(9.7); 0.834(16.0); 0.146(8.2); 0.081(0.5); 0.036(1.2); 0.027(2.3); 0.022(2.8); |
| | | | 0.008(56.1); 0.000(1644.1); −0.008(76.5); −0.025(1.4); −0.033(1.3); −0.045 |
| | | | (1.0); −0.052(0.8); −0.075(0.7); −0.091(0.3); −0.108(0.3); −0.141(0.6); −0.150(8.0) |
| I-123 | 2.99 | 3.13 | Example I-123: ¹H-NMR(400.0 MHz, d₆-DMSO): δ = |
| | | | 11.059(5.6); 9.159(4.8); 9.154(4.9); 9.006(1.5); 8.990(3.2); 8.975(1.6); 8.585(7.7); |
| | | | 8.581(7.9); 8.483(2.4); 8.478(2.4); 8.463(2.6); 8.458(2.7); 8.317(4.9); 8.092(8.6); |
| | | | 8.088(8.7); 8.081(5.4); 8.060(4.6); 7.843(8.8); 7.822(10.3); 7.561(8.8); |
| | | | 7.540(7.6); 4.663(6.3); 4.648(6.4); 3.429(0.3); 3.387(0.5); 3.378(0.6); 3.369(0.7); |
| | | | 3.331(645.6); 3.307(2.7); 3.278(0.5); 2.695(0.3); 2.676(3.7); 2.671(5.2); |
| | | | 2.667(3.9); 2.662(2.0); 2.641(0.4); 2.602(0.4); 2.550(1.2); 2.525(13.1); 2.520(20.4); |
| | | | 2.511(287.6); 2.507(600.0); 2.502(797.7); 2.498(587.3); 2.493(292.3); 2.414(0.5); |
| | | | 2.334(3.7); 2.329(5.2); 2.325(3.8); 2.083(0.6); 2.068(1.6); 2.052(2.3); |
| | | | 2.037(1.8); 2.021(0.7); 1.989(0.9); 1.398(1.4); 1.175(0.5); 1.148(0.6); 0.879(16.0); |
| | | | 0.866(8.7); 0.862(8.5); 0.146(5.0); 0.056(0.4); 0.047(0.5); 0.043(0.4); 0.038(0.4); |
| | | | 0.027(0.7); 0.020(1.5); 0.008(38.1); 0.000(1148.9); −0.008(52.0); −0.023(2.0); −0.031 |
| | | | (1.3); −0.033(1.3); −0.041(0.6); −0.045(0.4); −0.052(0.8); −0.150(5.1) |
| I-124 | 3.78 | 3.87 | Example I-124: ¹H-NMR(400.0 MHz, d₆-DMSO): δ = |
| | | | 10.991(0.7); 8.938(0.4); 8.449(0.9); 7.884(0.5); 7.881(0.9); 7.877(0.5); 7.836(1.1); |
| | | | 7.832(0.7); 7.816(1.3); 7.810(0.9); 7.788(0.4); 7.555(1.0); 7.534(0.9); 5.754(16.0); |
| | | | 4.653(0.7); 4.638(0.8); 3.321(13.1); 2.520(0.5); 2.512(6.6); 2.507(13.6); |
| | | | 2.503(18.1); 2.498(13.4); 2.494(6.6); 1.989(0.4); 0.861(1.8); 0.846(1.3); 0.000(3.2) |
| I-125 | 4.20 | 4.46 | Example I-125: ¹H-NMR(400.0 MHz, d₆-DMSO): δ = |
| | | | 10.929(5.6); 8.833(7.0); 8.829(9.2); 8.817(9.1); 8.496(5.1); 8.370(1.8); 8.351(3.6); |
| | | | 8.331(2.0); 8.316(3.1); 8.308(3.3); 8.297(3.3); 7.936(7.8); 7.932(7.8); 7.901(8.5); |
| | | | 7.896(3.6); 7.879(14.1); 7.865(6.6); 7.861(6.4); 7.845(4.5); 7.542(9.2); |
| | | | 7.532(6.9); 7.526(5.8); 7.521(11.7); 7.512(6.3); 7.502(5.2); 7.481(5.1); 7.329 |
| | | | (9.3); 7.308(7.6); 7.143(4.6); 7,122(4.1); 4.309(0.4); 4.291(0.4); 4.245(0.4); |
| | | | 4.235(0.4); 4.162(0.5); 4.140(0.6); 4.122(0.6); 4.085(0.6); 4.082(0.6); 3.956(1.1); |
| | | | 3.864(11.2); 3.778(16.0); 3.749(3.3); 3.684(3.8); 3.657(3.7); 3.519(2.0); |
| | | | 3.389(1.3); 3.322(0.7); 3.282(0.6); 3.269(0.6); 3.251(0.5); 3.199(0.4); 3.183(0.4); |
| | | | 3.144(0.4); 2.939(0.5); 2.929(1.1); 2.918(1.4); 2.911(2.2); 2.901(2.2); 2.893(1.5); |
| | | | 2.882(1.1); 2.872(0.5); 2.675(4.6); 2.671(6.4); 2.666(4.7); 2.599(0.3); 2.577(0.4); |
| | | | 2.524(19.3); 2.510(378.2); 2.506(741.5); 2.502(969.0); 2.497(724.0); 2.493(370.8); |
| | | | 2.356(0.5); 2.333(4.6); 2.328(6.3); 2.324(4.8); 2.297(0.3); 1.909(0.5); |
| | | | 1.308(0.4); 1.264(0.3); 1.249(0.8); 1.234(1.6); 0.992(0.4); 0.975(0.4); 0.855 |
| | | | (0.4); 0.838(0.4); 0.803(1.3); 0.790(3.9); 0.785(5.4); 0.773(5.0); 0.768(4.3); |
| | | | 0.756(1.8); 0.705(0.4); 0.640(1.8); 0.629(5.3); 0.623(5.0); 0.613(4.5); 0.601(1.3); |
| | | | 0.146(1.4); 0.008(14.0); 0.000(344.5); −0.008(16.7); −0.047(0.4); −0.150(1.6) |
| I-126 | | 3.63 | Example I-126: ¹H-NMR(400.0 MHz, d₆-DMSO): δ = |
| | | | 10.955(5.1); 8.848(6.2); 8.516(7.3); 8.513(7.4); 7.911(16.0); 7.907(10.8); 7.894(3.1); |
| | | | 7.889(9.8); 7.544(6.1); 7.524(5.5); 3.526(0.4); 3.505(0.4); 3.499(0.4); 3.485(0.5); |
| | | | 3.472(0.5); 3.456(0.3); 3.440(0.5); 3.425(0.6); 3.410(0.8); 3.373(1.5); |
| | | | 3.323(1458.6); 3.280(1.3); 3.266(0.5); 3.260(0.5); 3.250(0.6); 3.052(13.9); 2.994(3.3); |
| | | | 2.710(1.0); 2.675(4.2); 2.670(5.8); 2.666(4.4); 2.591(0.8); 2.541(234.7); |
| | | | 2.524(14.4); 2.519(22.5); 2.510(323.0); 2.506(657.5); 2.501(869.1); 2.497 |
| | | | (640.2); 2.492(314.9); 2.438(0.4); 2.430(0.5); 2.367(1.0); 2.333(3.8); 2.328(5.4); |
| | | | 2.324(4.0); 2.319(1.9); 2.290(0.6); 2.169(0.5); 2.054(0.5); 2.039(1.9); 2.024 |
| | | | (2.5); 2.008(2.1); 1.993(0.5); 1.258(0.5); 1.249(2.0); 1.233(4.7); 1.226(6.4); |
| | | | 1.217(4.3); 1.184(4.4); 1.175(5.9); 1.168(4.5); 1.152(1.8); 0.875(14.9); |
| | | | 0.860(12.9); 0.839(0.6); 0.008(0.5); 0.000(14.6) |
| I-127 | | 3.02 | Example I-127: ¹H-NMR(400.0 MHz, d₆-DMSO): δ = |
| | | | 10.981(5.1); 8.507(7.3); 8.503(7.2); 8.314(0.4); 8.300(1.0); 8.290(2.2); 8.277(2.2); |
| | | | 7.935(8.0); 7.931(7.9); 7.914(1.2); 7.907(8.9); 7.902(3.1); 7.890(3.3); 7.885(10.1); |
| | | | 7.878(1.2); 7.543(6.1); 7.523(5.4); 7.199(0.5); 7.072(0.6); 6.943(0.5); |
| | | | 3.807(0.7); 3.581(0.3); 3.547(0.4); 3.515(0.4); 3.496(0.3); 3.473(0.6); 3.469(0.5); |
| | | | 3.459(0.3); 3.453(0.6); 3.442(0.6); 3.434(0.7); 3.414(1.1); 3.402(1.3); 3.385(1.4); |
| | | | 3.374(1.8); 3.354(4.7); 3.322(2912.0); 3.294(4.2); 3.281(1.7); 3.276(1.7); |
| | | | 3.263(0.8); 3.256(0.6); 3.250(0.6); 3.218(0.4); 3.206(0.4); 3.019(0.6); 2.994(1.5); |
| | | | 2.950(0.5); 2.891(15.2); 2.878(15.2); 2.710(0.6); 2.679(3.3); 2.675(6.9); |
| | | | 2.670(9.6); 2.666(7.1); 2.661(3.5); 2.585(0.9); 2.581(0.9); 2.576(0.8); 2.541(179.5); |
| | | | 2.524(22.0); 2.510(573.4); 2.506(1153.9); 2.501(1513.2); 2.497(1116.3); |
| | | | 2.492(552.6); 2.454(2.4); 2.397(0.6); 2.367(1.3); 2.337(3.6); 2.332(7.2); |
| | | | 2.328(9.9); 2.323(7.3); 2.289(1.1); 2.285(0.5); 2.266(0.4); 2.248(0.3); 2.215(0.3); |
| | | | 2.071(0.6); 2.056(1.8); 2.040(2.3); 2.025(1.9); 2.009(0.8); 1.434(0.4); 1.298(0.3); |
| | | | 1.259(0.5); 1.244(1.0); 1.236(1.4); 0.874(16.0); 0.858(9.3); 0.000(20.1) |
| I-128 | | 3.84 | Example I-128: ¹H-NMR(400.0 MHz, d₆-DMSO): δ = |
| | | | 10.976(3.4); 8.509(4.2); 8.506(4.5); 8.484(3.7); 7.915(4.4); 7.912(4.7); 7.901(4.9); |
| | | | 7.879(5.6); 7.541(3.9); 7.520(3.5); 3.388(0.3); 3.369(0.5); 3.362(0.7); 3.355(1.0); |
| | | | 3.324(482.2); 3.290(0.5); 2.671(1.6); 2.667(1.3); 2.541(11.4); 2.506(196.5); |
| | | | 2.502(257.2); 2.498(200.3); 2.329(1.6); 2.033(1.2); 2.017(1.6); 2.002(1.3); |
| | | | 1.986(0.4); 1.407(16.0); 0.872(9.1); 0.857(8.2); 0.813(1.1); 0.796(4.4); 0.785(1.8); |
| | | | 0.742(0.4); 0.702(1.9); 0.691(4.3); 0.687(4.3); 0.674(1.2); 0.000(3.4) |
| I-129 | | 3.84 | Example I-129: ¹H-NMR(400.0 MHz, d₆-DMSO): δ = |
| | | | 20.011(0.7); 11.022(2.4); 8.523(3.3); 8.519(3.7); 8.085(1.3); 8.063(1.3); 7.948 |
| | | | (3.6); 7.944(3.9); 7.906(4.1); 7.884(5.0); 7.542(2.8); 7.522(2.5); 4.129(0.9); |
| | | | 4.108(0.8); 4.092(0.6); 3.322(2167.2); 3.123(0.8); 2.994(0.9); 2.710(0.8); 2.675(5.5); |
| | | | 2.670(7.4); 2.666(5.6); 2.540(119.3); 2.523(19.0); 2.510(410.4); 2.506(839.7); |
| | | | 2.501(1114.0); 2.497(819.0); 2.492(398.0); 2.367(0.5); 2.332(5.1); 2.328(7.1); |
| | | | 2.323(5.2); 2.289(0.6); 2.055(0.9); 2.040(1.1); 2.025(0.9); 1.234(16.0); |
| | | | 1.218(14.7); 0.876(7.4); 0.861(5.0); 0.000(19.2) |
| I-130 | | 3.84 | Example I-130: ¹H-NMR(400.0 MHz, d₆-DMSO): δ = |
| | | | 11.076(5.5); 9.942(0.4); 8.554(8.0); 8.550(8.2); 8.508(3.4); 8.487(3.4); 7.955(8.8); |
| | | | 7.951(8.7); 7.939(0.4); 7.917(9.1); 7.911(3.3); 7.899(3.4); 7.894(10.5); 7.887(1.3); |
| | | | 7.545(6.6); 7.525(5.8); 4.901(0.4); 4.890(1.2); 4.884(1.5); 4.872(1.5); |
| | | | 4.867(2.0); 4.851(1.5); 4.846(1.3); 4.834(0.4); 4.828(0.3); 3.477(0.3); 3.467(0.4); |
| | | | 3.442(0.4); 3.431(0.4); 3.408(0.6); 3.363(2.5); 3.339(16.7); 3.323(1220.5); |
| | | | 2.994(1.5); 2.711(1.0); 2.675(3.4); 2.670(4.5); 2.666(3.4); 2.661(1.7); 2.639(0.3); |
| | | | 2.592(0.6); 2.570(1.7); 2.541(239.9); 2.524(10.9); 2.510(259.9); 2.506(526.2); |
| | | | 2.501(692.6); 2.497(511.9); 2.492(252.3); 2.367(0.8); 2.333(3.1); 2.328 |
| | | | (4.3); 2.324(3.2); 2.290(0.4); 2.065(0.6); 2.050(1.8); 2.034(2.4); 2.019(2.0); |
| | | | 2.003(0.6); 1.464(15.1); 1.447(15.1); 1.245(0.3); 1.235(0.7); 0.881(16.0); |
| | | | 0.866(11.0); 0.000(10.7); −0.008(0.4) |
| I-131 | | 3.15 | Example I-131: ¹H-NMR(400.0 MHz, d₆-DMSO): δ = |
| | | | 10.991(5.3); 8.993(1.6); 8.978(3.3); 8.963(1.6); 8.541(7.7); 8.537(7.7); 7.979(8.6); |
| | | | 7.975(8.5); 7.937(0.9); 7.930(9.1); 7.925(3.1); 7.913(3.2); 7.908(10.2); 7.901(1.2); |
| | | | 7.550(6.1); 7.530(5.5); 4.415(9.0); 4.400(9.0); 3.441(0.4); 3.410(0.5); |
| | | | 3.396(0.6); 3.386(0.5); 3.380(0.6); 3.369(0.9); 3.359(1.7); 3.324(971.7); 3.296(1.7); |
| | | | 3.283(0.9); 3.272(0.6); 3.265(0.5); 2.994(2.2); 2.711(0.6); 2.680(1.2); 2.675(2.4); |
| | | | 2.671(3.4); 2.666(2.5); 2.662(1.3); 2.575(0.5); 2.541(172.5); 2.524(8.2); |
| | | | 2.519(12.7); 2.511(189.4); 2.506(387.8); 2.502(513.4); 2.497(377.8); 2.493(185.6); |
| | | | 2.461(0.8); 2.367(0.6); 2.337(1.1); 2.333(2.3); 2.328(3.2); 2.324(2.4); |
| | | | 2.320(1.1); 2.294(0.4); 2.104(0.6); 2.088(1.6); 2.073(2.2); 2.058(1.8); 2.042 |
| | | | (0.6); 1.236(0.4); 0.880(16.0); 0.866(8.5); 0.863(8.2); 0.000(7.7) |
| I-132 | | 3.78 | Example I-132: ¹H-NMR(400.0 MHz, d₆-DMSO): δ = |
| | | | 10.996(4.3); 8.518(6.3); 8.514(6.1); 8.319(1.3); 8.304(2.5); 8.289(1.2); 7.946(6.9); |
| | | | 7.942(6.6); 7.907(7.4); 7.902(2.5); 7.890(3.0); 7.885(8.3); 7.878(1.0); 7.543(5.2); |
| | | | 7.523(4.7); 3.372(0.5); 3.325(340.1); 3.293(2.3); 2.995(0.6); 2.711(0.4); |
| | | | 2.676(0.9); 2.671(1.2); 2.667(0.9); 2.541(87.4); 2.524(4.1); 2.511(77.0); |
| | | | 2.507(146.0); 2.502(185.9); 2.498(134.4); 2.493(65.4); 2.368(0.4); 2.333(0.9); |
| | | | 2.329(1.2); 2.325(0.9); 2.074(0.5); 2.059(1.4); 2.043(1.9); 2.028(1.6); 2.012(0.5); |
| | | | 1.621(0.5); 1.603(2.3); 1.584(4.4); 1.567(4.5); 1.549(2.5); 1.530(0.6); 0.947(7.8); |
| | | | 0.928(16.0); 0.910(7.0); 0.877(13.5); 0.863(7.9); 0.000(2.9) |
| I-133 | | 4.00 | Example I-133: ¹H-NMR(400.0 MHz, d₆-DMSO): δ = |
| | | | 11.005(5.5); 8.515(7.6); 8.512(7.8); 8.482(3.0); 8.462(3.0); 7.916(8.3); 7.912(8.6); |
| | | | 7.902(9.0); 7.897(3.2); 7.885(3.2); 7.880(10.3); 7.873(1.2); 7.540(6.3); 7.520(5.6); |
| | | | 4.483(1.0); 4.462(2.0); 4.442(2.1); 4.421(1.1); 3.383(0.5); 3.376(0.6); |
| | | | 3.366(0.7); 3.354(1.2); 3.323(746.4); 3.287(0.5); 2.995(1.3); 2.675(1.6); 2.671(2.2); |
| | | | 2.666(1.7); 2.662(0.8); 2.558(0.5); 2.541(33.3); 2.524(5.2); 2.510(126.0); |
| | | | 2.506(256.2); 2.502(339.9); 2.497(251.5); 2.493(125.2); 2.457(0.3); 2.333(1.6); |
| | | | 2.328(2.3); 2.324(1.9); 2.312(1.1); 2.304(1.5); 2.292(2.5); 2.284(2.4); 2.275(2.5); |
| | | | 2.269(2.3); 2.264(2.1); 2.254(1.2); 2.247(1.0); 2.119(0.7); 2.112(0.6); |
| | | | 2.095(2.2); 2.070(3.3); 2.063(3.9); 2.046(4.3); 2.031(2.3); 2.017(1.0); 1.756(1.2); |
| | | | 1.743(2.3); 1.733(2.5); 1.717(3.1); 1.699(1.8); 1.691(1.5); 1.673(0.6); 0.879 |
| | | | (16.0); 0.864(11.7); 0.000(3.5) |
| I-134 | | 3.58 | Example I-134: ¹H-NMR(400.0 MHz, d₆-DMSO): δ = |
| | | | 11.078(5.9); 8.710(7.3); 8.572(7.7); 8.568(7.8); 7.972(8.6); 7.969(8.6); 7.946(1.0); |
| | | | 7.939(9.2); 7.934(3.2); 7.922(3.3); 7.917(10.6); 7.910(1.1); 7.551(6.4); 7.531(5.8); |
| | | | 3.955(1.6); 3.946(3.4); 3.936(1.9); 3.924(2.3); 3.915(4.1); 3.905(1.9); |
| | | | 3.651(2.6); 3.624(4.5); 3.599(2.3); 3.594(2.0); 3.390(0.4); 3.385(0.4); 3.381(0.5); |
| | | | 3.372(0.6); 3.364(0.8); 3.324(731.3); 3.293(0.4); 2.995(3.0); 2.711(0.7); |
| | | | 2.675(1.5); 2.671(2.1); 2.666(1.6); 2.662(0.8); 2.589(0.3); 2.541(176.6); |
| | | | 2.524(5.1); 2.511(115.3); 2.506(234.8); 2.502(311.2); 2.497(228.3); 2.493(111.5); |
| | | | 2.451(3.1); 2.417(3.5); 2.367(0.7); 2.333(1.4); 2.328(1.9); 2.324(1.4); 2.092(0.6); |
| | | | 2.076(3.5); 2.061(3.4); 2.045(4.0); 2.032(2.3); 2.016(1.7); 2.006(1.4); 0.883(16.0); |
| | | | 0.868(11.0); 0.000(4.7) |
| I-135 | | 3.11 | Example I-135: ¹H-NMR(400.0 MHz, d₆-DMSO): δ = |
| | | | 10.990(6.6); 8.868(4.0); 8.847(4.1); 8.534(9.1); 8.530(9.2); 7.969(10.3); 7.965 |
| | | | (10.2); 7.932(1.2); 7.925(10.5); 7.920(3.9); 7.908(4.0); 7.903(12.2); 7.896(1.4); |
| | | | 7.548(7.3); 7.528(6.6); 4.956(1.4); 4.934(2.4); 4.929(2.0); 4.913(1.9); 4.907(2.6); |
| | | | 4.885(1.4); 4.456(1.4); 4.437(3.6); 4.434(3.6); 4.415(2.1); 4.344(1.4); 4.328(1.9); |
| | | | 4.322(1.7); 4.317(2.0); 4.306(1.5); 4.301(2.1); 4.296(1.7); 4.280(1.1); |
| | | | 3.327(241.9); 3.294(0.4); 2.996(2.0); 2.712(0.5); 2.676(0.6); 2.672(0.8); 2.667 |
| | | | (0.6); 2.569(0.9); 2.565(1.0); 2.542(144.2); 2.525(3.4); 2.512(49.1); 2.507(98.0); |
| | | | 2.503(129.1); 2.498(95.5); 2.494(46.9); 2.468(0.4); 2.455(1.0); 2.432(1.4); |
| | | | 2.428(1.9); 2.405(2.1); 2.399(1.8); 2.377(1.5); 2.370(1.2); 2.348(0.5); 2.334(0.6); |
| | | | 2.329(0.8); 2.325(0.7); 2.108(0.6); 2.094(1.5); 2.091(1.7); 2.084(1.3); 2.077 |
| | | | (2.7); 2.063(1.7); 2.060(1.8); 2.046(0.7); 0.877(16.0); 0.866(8.7); 0.858(7.4); |
| | | | 0.835(0.4); 0.000(2.6) |
| I-136 | | 3.68 | Example I-136: ¹H-NMR(400.0 MHz, d₆-DMSO): δ = |
| | | | 11.082(2.3); 8.562(3.3); 8.558(4.0); 8.536(1.5); 7.960(3.3); 7.957(3.3); 7.915(3.6); |
| | | | 7.910(1.4); 7.893(4.2); 7.547(2.6); 7.526(2.4); 4.629(1.1); 4.611(1.6); 4.593(1.1); |
| | | | 3.693(16.0); 3.350(0.7); 3.324(157.1); 2.995(0.4); 2.675(0.4); 2.671(0.6); |
| | | | 2.667(0.4); 2.541(41.6); 2.506(65.0); 2.502(85.7); 2.498(64.5); 2.333(0.4); |
| | | | 2.329(0.5); 2.324(0.4); 2.077(0.7); 2.062(1.0); 2.046(0.8); 1.474(6.4); 1.456(6.4); |
| | | | 0.884(6.6); 0.870(3.8); 0.000(1.4) |
| I-137 | | 4.17 | Example I-137: ¹H-NMR(400.0 MHz, d₆-DMSO): δ = |
| | | | 11.000(5.6); 8.953(6.7); 8.547(8.1); 8.543(8.1); 7.942(8.9); 7.939(8.7); 7.916(9.3); |
| | | | 7.911(3.4); 7.899(3.5); 7.894(10.5); 7.546(6.4); 7.526(5.9); 3.507(0.3); 3.456(0.3); |
| | | | 3.438(0.3); 3.433(0.4); 3.419(0.5); 3.404(0.7); 3.390(0.8); 3.372(1.7); |
| | | | 3.361(1.8); 3.322(1660.5); 3.295(2.6); 3.289(2.0); 3.262(0.4); 3.249(0.3); |
| | | | 2.994(0.5); 2.675(4.5); 2.670(6.2); 2.666(4.6); 2.591(0.4); 2.585(0.5); 2.541(27.4); |
| | | | 2.524(14.7); 2.510(359.6); 2.506(725.2); 2.501(952.7); 2.497(705.4); 2.493(349.7); |
| | | | 2.431(0.5); 2.333(4.4); 2.328(6.1); 2.324(4.4); 2.290(0.7); 2.064(0.6); 2.048(2.0); |
| | | | 2.033(2.7); 2.018(2.3); 2.002(0.6); 1.433(0.4); 1.402(2.0); 1.381(6.3); |
| | | | 1.367(3.2); 1.353(0.4); 1.328(0.9); 1.298(0.4); 1.270(5.3); 1.245(1.2); |
| | | | 1.236(0.9); 0.879(16.0); 0.864(13.6); 0.008(0.6); 0.000(17.3); −0.008(0.6) |
| I-138 | | 3.42 | Example I-138: ¹H-NMR(400.0 MHz, d₆-DMSO): δ = |
| | | | 10.993(2.5); 8.869(3.0); 8.535(3.3); 8.531(3.4); 7.931(3.6); 7.927(3.7); 7.913(3.8); |
| | | | 7.891(4.4); 7.547(2.8); 7.527(2.5); 3.620(16.0); 3.362(0.3); 3.325(151.6); |
| | | | 2.995(0.7); 2.676(0.4); 2.671(0.6); 2.667(0.5); 2.541(52.7); 2.524(1.6); 2.506(70.2); |
| | | | 2.502(91.3); 2.498(68.5); 2.333(0.4); 2.329(0.6); 2.325(0.4); 2.059(0.8); |
| | | | 2.043(1.0); 2.028(0.8); 1.527(1.1); 1.515(2.8); 1.507(3.1); 1.496(1.3); 1.283(1.3); |
| | | | 1.271(3.0); 1.263(2.9); 1.251(1.1); 0.877(7.0); 0.863(4.3); 0.000(1.6) |
| I-139 | | 4.28 | Example I-139: ¹H-NMR(400.0 MHz, d₆-DMSO): δ = |
| | | | 11.017(3.4); 8.564(4.2); 8.560(4.5); 8.358(4.1); 7.956(4.5); 7.952(4.7); 7.928(4.9); |
| | | | 7.906(5.7); 7.551(3.7); 7.531(3.4); 3.374(0.5); 3.359(0.9); 3.323(490.0); |
| | | | 3.284(0.5); 3.280(0.5); 2.994(0.8); 2.711(0.4); 2.675(1.3); 2.670(1.8); 2.666(1.4); |
| | | | 2.541(89.4); 2.523(5.1); 2.506(204.9); 2.501(272.0); 2.497(210.5); 2.367(0.4); |
| | | | 2.333(1.3); 2.328(1.7); 2.324(1.4); 2.134(0.6); 2.116(1.4); 2.099(3.5); 2.085 |
| | | | (3.8); 2.081(3.9); 2.067(3.9); 2.050(2.2); 2.039(1.8); 2.032(1.2); 2.024(1.4); |
| | | | 2.008(0.5); 1.051(7.3); 1.033(16.0); 1.014(7.0); 0.890(8.6); 0.875(8.3); 0.000(4.1) |
| I-140 | | 3.58 | Example I-140: ¹H-NMR(400.0 MHz, d₆-DMSO): δ = |
| | | | 11.020(5.8); 9.096(6.6); 8.546(8.5); 8.542(8.5); 7.956(9.2); 7.952(9.0); 7.922(1.1); |
| | | | 7.915(9.8); 7.909(3.3); 7.898(3.4); 7.893(11.1); 7.885(1.2); 7.703(0.3); 7.547(6.5); |
| | | | 7.527(5.9); 7.362(0.6); 7.341(0.9); 7.216(0.8); 7.200(0.3); 7.195(0.6); |
| | | | 3.457(0.4); 3.379(0.5); 3.369(0.7); 3.324(634.0); 3.296(1.4); 3.282(43.4); |
| | | | 2.995(1.6); 2.711(0.7); 2.680(0.8); 2.676(1.5); 2.671(2.1); 2.666(1.6); 2.662(0.8); |
| | | | 2.565(0.4); 2.541(168.0); 2.524(4.9); 2.520(7.8); 2.511(118.6); 2.506(243.1); |
| | | | 2.502(321.9); 2.497(235.0); 2.493(113.9); 2.367(0.6); 2.337(0.6); 2.333(1.4); |
| | | | 2.329(2.0); 2.324(1.5); 2.320(0.7); 2.053(0.5); 2.038(2.1); 2.022(2.8); 2.007(2.3); |
| | | | 1.991(0.6); 1.131(2.3); 1.117(6.0); 1.112(7.1); 1.100(3.4); 1.058(0.5); 1.045 |
| | | | (0.5); 1.004(3.4); 0.991(6.8); 0.987(6.5); 0.972(2.2); 0.952(1.2); 0.935(1.2); |
| | | | 0.883(16.0); 0.867(14.1); 0.701(1.1); 0.684(1.1); 0.000(6.3) |
| I-141 | | 3.84 | Example I-141: ¹H-NMR(400.0 MHz, d₆-DMSO): δ = |
| | | | 11.046(5.4); 8.532(7.0); 8.528(7.3); 8.357(1.6); 8.342(3.1); 8.327(1.6); 7.964(7.6); |
| | | | 7.960(7.7); 7.910(8.4); 7.889(9.7); 7.857(0.4); 7.544(6.5); 7.524(5.8); 3.440(0.4); |
| | | | 3.405(0.5); 3.384(0.7); 3.370(1.2); 3.361(1.5); 3.355(1.8); 3.323(972.5); |
| | | | 3.282(0.8); 3.269(0.6); 3.251(4.7); 3.235(7.3); 3.219(4.6); 2.994(1.1); 2.710 |
| | | | (0.6); 2.675(2.6); 2.671(3.7); 2.666(2.8); 2.588(0.4); 2.579(0.4); 2.541(136.3); |
| | | | 2.524(8.7); 2.506(416.1); 2.501(552.3); 2.497(420.3); 2.431(0.4); 2.367(0.7); |
| | | | 2.333(2.5); 2.328(3.5); 2.324(2.7); 2.290(0.4); 2.084(0.7); 2.069(1.8); 2.053(2.4); |
| | | | 2.038(2.0); 2.022(0.6); 1.244(0.3); 1.235(0.5); 1.146(0.4); 1.089(0.6); 1.078 |
| | | | (1.2); 1.072(1.1); 1.060(1.8); 1.048(1.1); 1.041(1.2); 1.029(0.7); 1.022(0.5); |
| | | | 0.879(16.0); 0.865(9.2); 0.843(1.0); 0.514(1.5); 0.503(4.7); 0.499(5.2); 0.489(2.4); |
| | | | 0.483(4.9); 0.479(4.9); 0.469(1.8); 0.295(1.8); 0.282(6.1); 0.269(6.1); 0.258 |
| | | | (1.5); 0.000(9.3) |
| I-142 | 3.86 | 4.07 | Example I-142: ¹H-NMR(400.0 MHz, d₆-DMSO): δ = |
| | | | 10.901(2.5); 8.930(0.7); 8.914(1.5); 8.898(0.7); 8.516(3.2); 8.512(3.3); 8.313(1.7); |
| | | | 7.964(3.6); 7.960(3.7); 7.837(3.9); 7.816(4.6); 7.738(3.6); 7.716(4.5); 7.555(3.9); |
| | | | 7.534(3.6); 7.525(4.7); 7.503(3.8); 7.431(0.4); 4.654(2.8); 4.638(2.8); |
| | | | 3.318(50.2); 3.294(0.7); 2.675(0.4); 2.671(0.6); 2.666(0.4); 2.524(1.6); |
| | | | 2.511(31.9); 2.506(64.4); 2.502(85.6); 2.497(64.3); 2.493(32.7); 2.376(0.3); 2.365(0.7); |
| | | | 2.358(0.8); 2.347(1.4); 2.336(1.0); 2.329(1.3); 2.318(0.5); 2.051(0.5); 2.046 |
| | | | (0.6); 2.041(0.5); 2.034(1.0); 2.021(0.6); 2.016(0.6); 1.989(0.9); 1.398(16.0); |
| | | | 1.175(0.5); 1.170(0.7); 1.158(2.1); 1.153(2.4); 1.140(2.2); 1.135(2.1); 1.123(0.7); |
| | | | 0.862(4.4); 0.843(2.5); 0.643(0.7); 0.632(2.6); 0.628(2.3); 0.621(2.2); 0.617(2.7); |
| | | | 0.605(0.7); 0.000(2.9) |
| I-143 | | 3.24 | Example I-143: ¹H-NMR(400.0 MHz, d₆-DMSO): δ = |
| | | | 11.055(5.9); 8.986(1.6); 8.970(3.4); 8.955(1.6); 8.562(1.9); 8.531(7.6); 8.519(2.2); |
| | | | 8.065(3.8); 8.048(3.4); 8.045(3.4); 7.992(7.5); 7.840(9.2); 7.819(10.9); 7.556(9.2); |
| | | | 7.535(8.3); 7.512(0.3); 4.658(6.8); 4.642(6.8); 3.370(0.6); 3.365(0.6); |
| | | | 3.356(0.8); 3.325(563.6); 2.995(1.6); 2.711(0.7); 2.680(0.6); 2.676(1.3); 2.671(1.8); |
| | | | 2.666(1.4); 2.662(0.7); 2.561(0.5); 2.541(197.1); 2.524(3.9); 2.520(6.0); |
| | | | 2.511(94.5); 2.506(199.5); 2.502(269.8); 2.497(202.0); 2.493(101.7); 2.367(0.7); |
| | | | 2.338(0.5); 2.333(1.2); 2.329(1.7); 2.324(1.3); 2.320(0.6); 2.073(0.6); 2.058(2.1); |
| | | | 2.042(2.8); 2.027(2.4); 2.011(0.7); 1.235(0.7); 0.871(16.0); 0.856(13.9); 0.000(3.3) |
| I-144 | 3.00 | 3.55 | Example I-144: ¹H-NMR(400.0 MHz, d₆-DMSO): δ = |
| | | | 20.009(0.7); 10.933(7.3); 8.941(2.2); 8.925(4.3); 8.910(2.1); 8.535(8.9); 8.532 |
| | | | (8.9); 8.314(5.7); 8.071(1.8); 7.983(9.7); 7.979(9.7); 7.837(10.8); 7.816(13.1); |
| | | | 7.799(9.7); 7.778(11.3); 7.556(12.6); 7.549(11.2); 7.536(12.0); 7.529(9.4); |
| | | | 7.438(0.7); 7.418(0.6); 4.656(8.5); 4.640(8.5); 4.361(0.7); 3.794(2.1); 3.765(5.9); |
| | | | 3.736(6.3); 3.708(2.2); 3.390(1.4); 3.373(1.8); 3.316(867.9); 3.240(0.6); 3.184 |
| | | | (0.7); 2.862(0.6); 2.750(0.6); 2.675(12.2); 2.670(16.0); 2.666(12.3); 2.631(1.5); |
| | | | 2.506(1864.0); 2.501(2393.4); 2.497(1817.3); 2.409(1.3); 2.332(11.3); 2.328(15.2); |
| | | | 2.324(11.6); 2.038(2.9); 2.024(2.0); 2.007(1.1); 1.776(1.0); 1.757(0.6); |
| | | | 1.352(1.5); 1.335(1.0); 1.298(1.2); 1.259(1.5); 1.250(1.9); 1.234(3.4); 1.206(2.8); |
| | | | 1.193(3.7); 1.176(2.7); 1.077(2.2); 1.065(1.9); 1.054(4.6); 1.036(4.2); 1.015(2.2); |
| | | | 1.005(2.6); 0.866(15.5); 0.858(11.2); 0.847(8.3); 0.699(2.5); 0.681(2.0); |
| | | | 0.146(8.2); 0.000(1723.0); −0.150(8.2) |
| I-145 | 4.72 | 4.90 | Example I-145: ¹H-NMR(601.6 MHz, d₆-DMSO): δ = |
| | | | 11.052(5.4); 8.528(6.8); 8.526(6.9); 8.130(3.0); 8.116(3.1); 7.944(8.1); 7.942(8.1); |
| | | | 7.905(1.2); 7.900(8.8); 7.896(3.1); 7.889(3.3); 7.885(9.8); 7.880(1.1); 7.538(5.9); |
| | | | 7.524(5.6); 3.834(0.4); 3.822(0.9); 3.816(1.0); 3.809(1.0); 3.803(1.1); |
| | | | 3.790(0.6); 3.784(0.4); 3.323(295.7); 2.617(0.9); 2.614(1.2); 2.611(0.9); 2.523 |
| | | | (2.1); 2.520(2.6); 2.517(2.7); 2.508(68.8); 2.505(141.9); 2.502(190.3); 2.499(140.8); |
| | | | 2.496(67.6); 2.389(0.9); 2.386(1.2); 2.383(0.9); 2.331(0.8); 2.316(0.8); 2.057(0.7); |
| | | | 2.037(4.8); 2.026(3.7); 1.952(2.1); 1.935(3.4); 1.466(0.6); 1.445(2.0); |
| | | | 1.425(5.9); 1.407(5.9); 1.392(1.7); 1.388(1.9); 1.366(0.8); 1.352(1.1); 1.298(0.7); |
| | | | 1.259(1.1); 1.249(0.4); 1.232(1.4); 1.159(2.2); 1.070(2.1); 0.892(0.4); 0.873(16.0); |
| | | | 0.864(8.1); 0.861(7.6); 0.841(0.5); 0.005(0.4); 0.000(11.5); −0.006(0.4) |
| I-146 | 4.60 | 4.77 | Example I-146: ¹H-NMR(601.6 MHz, d₆-DMSO): δ = |
| | | | 10.970(5.8); 8.539(7.5); 8.537(7.1); 8.211(3.2); 8.199(3.2); 7.939(8.7); 7.937(8.3); |
| | | | 7.908(1.1); 7.903(9.3); 7.900(3.3); 7.892(3.6); 7.888(10.3); 7.884(1.1); 7.541(6.2); |
| | | | 7.528(5.8); 5.753(0.5); 4.109(1.0); 4.102(1.6); 4.096(1.8); 4.090(1.7); |
| | | | 4.084(1.0); 3.367(2.4); 3.341(813.8); 3.311(1.2); 3.282(0.3); 2.618(0.9); 2.615(1.2); |
| | | | 2.612(0.9); 2.609(0.4); 2.524(2.2); 2.521(2.8); 2.518(3.0); 2.509(70.4); |
| | | | 2.507(142.1); 2.504(188.1); 2.501(136.6); 2.498(64.6); 2.443(0.5); 2.433(0.7); |
| | | | 2.426(1.0); 2.417(0.9); 2.410(1.0); 2.394(1.0); 2.391(1.2); 2.388(1.5); 2.385(1.1); |
| | | | 2.059(0.6); 2.048(2.1); 2.038(2.9); 2.028(2.3); 2.018(0.7); 1.916(1.8); 1.910(2.0); |
| | | | 1.902(1.7); 1.894(2.4); 1.887(2.5); 1.803(2.0); 1.796(2.1); 1.788(1.9); 1.781(2.6); |
| | | | 1.774(2.6); 1.768(1.6); 1.761(1.6); 1.755(1.6); 1.737(2.9); 1.720(1.3); |
| | | | 1.714(1.9); 1.708(1.1); 1.647(1.3); 1.641(1.3); 1.624(2.6); 1.606(2.3); 1.589(1.0); |
| | | | 1.585(0.8); 1.352(1.3); 1.298(1.0); 1.259(1.5); 1.233(1.9); 1.227(1.5); 1.159(1.4); |
| | | | 1.070(1.3); 0.894(15.3); 0.883(16.0); 0.864(0.5); 0.853(0.5); 0.640(0.3); |
| | | | 0.005(0.4); 0.000(10.9); −0.006(0.4) |
| I-147 | | 4.33 | Example I-147: ¹H-NMR(400.0 MHz, d₆-DMSO): δ = |
| | | | 9.465(2.4); 9.288(0.8); 9.273(1.5); 9.257(0.8); 8.471(3.0); 8.313(4.9); 8.089(0.5); |
| | | | 8.015(2.9); 8.011(3.2); 7.929(3.3); 7.907(3.7); 7.833(3.5); 7.812(4.3); 7.548 |
| | | | (5.9); 7.528(5.3); 7.466(0.7); 7.453(0.7); 7.399(0.4); 7.028(0.4); 4.662(2.7); |
| | | | 4.647(2.6); 4.611(0.5); 4.595(0.5); 4.557(0.4); 3.695(0.4); 3.508(0.6); 3.451(0.5); |
| | | | 3.314(834.9); 3.175(0.4); 3.062(0.5); 3.044(0.4); 3.020(0.5); 2.999(0.5); |
| | | | 2.952(2.0); 2.871(0.5); 2.839(0.6); 2.808(0.6); 2.767(0.8); 2.670(16.0); 2.666(13.0); |
| | | | 2.505(1722.0); 2.501(2246.0); 2.497(1813.0); 2.332(10.7); 2.328(14.3); 2.324(11.5); |
| | | | 1.470(10.3); 1.406(0.5); 1.256(1.2); 1.235(1.9); 1.185(3.2); 1.179(3.3); |
| | | | 0.969(0.5); 0.866(0.4); 0.854(0.6); 0.751(3.2); 0.745(3.4); 0.197(0.5); 0.146 |
| | | | (7.9); 0.083(1.3); 0.074(1.3); 0.000(1595.7); −0.150(7.2) |
| I-148 | 4.72 | 4.85 | Example I-148: ¹H-NMR(400.0 MHz, d₆-DMSO): δ = |
| | | | 11.001(3.8); 8.940(1.0); 8.924(2.2); 8.908(1.1); 8.314(1.0); 8.039(4.9); 8.036(4.9); |
| | | | 7.844(5.4); 7.824(6.3); 7.571(5.6); 7.551(4.9); 7.535(5.4); 7.532(5.3); 7.508(3.8); |
| | | | 7.493(3.7); 4.644(4.1); 4.629(4.2); 3.317(102.3); 2.675(0.8); 2.671(1.1); |
| | | | 2.667(0.8); 2.524(3.4); 2.506(123.1); 2.502(161.1); 2.497(120.4); 2.422(0.5); |
| | | | 2.405(0.8); 2.387(2.1); 2.369(2.9); 2.351(2.2); 2.333(1.6); 2.329(1.4); 2.324 |
| | | | (0.9); 2.319(0.6); 2.078(0.6); 2.054(15.4); 2.038(1.2); 2.021(1.5); 2.006(1.0); |
| | | | 1.989(1.2); 1.398(16.0); 1.175(0.5); 1.006(5.0); 0.987(10.6); 0.969(5.0); |
| | | | 0.841(4.1); 0.831(4.9); 0.822(8.0); 0.008(1.4); 0.000(39.0); −0.008(1.7) |
| I-149 | 4.14 | 4.25 | Example I-149: ¹H-NMR(400.0 MHz, d₆-DMSO): δ = |
| | | | 11.031(2.2); 8.979(0.6); 8.963(1.3); 8.948(0.6); 8.563(3.3); 8.559(3.2); |
| | | | 8.313(16.0); 8.047(3.7); 8.043(3.6); 7.997(1.0); 7.970(0.9); 7.927(0.6); 7.906(1.2); |
| | | | 7.890(1.1); 7.855(1.9); 7.839(4.6); 7.819(4.7); 7.559(4.1); 7.538(3.6); 4.660(2.5); |
| | | | 4.645(2.9); 3.317(58.7); 3.293(6.6); 2.675(0.7); 2.671(1.0); 2.666(0.8); 2.524 |
| | | | (3.1); 2.511(58.6); 2.506(115.2); 2.502(151.1); 2.497(112.9); 2.333(0.7); |
| | | | 2.329(1.0); 2.324(0.7); 2.063(0.7); 2.047(1.0); 2.032(0.8); 1.398(12.4); 0.877(7.3); |
| | | | 0.863(4.2); 0.860(4.0); 0.008(0.4); 0.000(11.1); −0.008(0.5) |
| I-150 | 4.41 | 4.51 | Example I-150: ¹H-NMR(400.0 MHz, d₆-DMSO): δ = |
| | | | 11.003(1.2); 8.966(0.4); 8.950(0.7); 8.934(0.4); 8.582(1.6); 8.579(1.7); 8.049(1.4); |
| | | | 8.029(3.4); 8.026(2.8); 7.866(1.4); 7.841(2.5); 7.820(2.3); 7.563(1.9); 7.542(1.7); |
| | | | 4.664(1.4); 4.648(1.4); 3.321(12.5); 2.526(0.4); 2.512(8.5); 2.508(17.1); |
| | | | 2.503(22.6); 2.499(17.0); 2.495(8.8); 2.049(0.5); 2.033(0.4); 1.398(16.0); |
| | | | 0.876(3.1); 0.865(1.7); 0.857(1.5); 0.000(0.8) |
| I-151 | 3.50 | 3.09 | Example I-151: ¹H-NMR(400.0 MHz, d₆-DMSO): δ = |
| | | | 11.066(0.6); 11.017(6.0); 8.971(2.0); 8.956(3.7); 8.940(1.7); 8.620(0.8); 8.617 |
| | | | (0.8); 8.589(7.3); 8.586(7.4); 8.314(0.6): 8.300(0.6); 8.279(1.1); 8.229(1.2); |
| | | | 8.207(0.7); 8.115(5.7); 8.094(12.9); 8.059(10.0); 8.039(12.3); 8.036(10.6); |
| | | | 7.840(9.7); 7.820(11.4); 7.561(9.7); 7.541(8.5); 5.754(11.7); 4.663(7.1); 4.648(7.1); |
| | | | 4.627(0.3); 4.038(0.6); 4.020(0.6); 3.318(137.5); 2.675(1.2); 2.671(1.6); |
| | | | 2.666(1.2); 2.662(0.6); 2.524(6.3); 2.510(93.7); 2.506(184.2); 2.502(242.4); |
| | | | 2.497(179.6); 2.493(91.8); 2.333(1.2); 2.328(1.6); 2.324(1.2); 2.080(0.6); 2.065(1.7); |
| | | | 2.049(2.4); 2.034(1.9); 2.018(0.7); 1.988(2.5); 1.234(0.5); 1.193(0.7); 1.175(1.3); |
| | | | 1.157(0.7); 0.876(16.0); 0.863(9.1); 0.858(8.0); 0.833(0.6); 0.146(2.0); 0.007(17.3); |
| | | | 0.000(390.7); −0.009(20.6); −0.041(0.6); −0.150(2.0) |
| I-152 | 4.05 | 4.14 | Example I-152: ¹H-NMR(400.0 MHz, d₆-DMSO): δ = |
| | | | 11.017(4.4); 8.977(1.2); 8.961(2.5); 8.945(1.2); 8.491(5.5); 8.487(5.3); 8.314(0.6); |
| | | | 8.015(5.8); 8.011(5.5); 7.889(5.5); 7.866(5.6); 7.838(6.3); 7.817(7.3); 7.555(6.5); |
| | | | 7.535(5.6); 4.654(5.0); 4.639(4.9): 3.317(134.7); 2.675(1.2); 2.671(1.6); |
| | | | 2.666(1.2); 2.506(185.6); 2.502(235.9); 2.497(173.3); 2.333(1.2); 2.328(1.5); |
| | | | 2.324(1.1); 2.071(0.4); 2.056(1.5); 2.040(1.9); 2.025(1.6); 2.009(0.5); 1.398 |
| | | | (16.0); 0.893(0.5); 0.873(11.6); 0.858(9.5); 0.146(1.6); 0.046(0.5); 0.008(16.3); |
| | | | 0.000(330.4); −0.008(16.3); −0.042(0.4); −0.150(1.7) |
| I-153 | 3.48 | 3.60 | Example I-153: ¹H-NMR(400.0 MHz, d₆-DMSO): δ = |
| | | | 7.983(0.3); 7.893(0.3); 7.872(0.4); 7.837(0.3); 7.817(0.4); 7.559(0.3); 5.751(16.0); |
| | | | 3.317(1.4); 2.507(4.9); 2.503(6.2); 2.498(4.6); 0.869(0.4); 0.862(0.3); 0.008(0.6); |
| | | | 0.000(10.5); −0.008(0.6) |
| I-154 | 3.61 | 3.76 | Example I-154: ¹H-NMR(400.0 MHz, d₆-DMSO): δ = |
| | | | 10.948(4.3); 8.950(1.2); 8.934(2.5); 8.919(1.2); 8.478(4.9); 8.474(5.4); 8.313(1.0); |
| | | | 7.960(5.2); 7.957(5.8); 7.885(4.7); 7.882(5.1); 7.837(6.0); 7.816(7.2); 7.625(1.5); |
| | | | 7.621(1.6); 7.604(3.5); 7.600(3.8); 7.575(6.1); 7.554(8.6); 7.533(5.6); |
| | | | 4.652(4.9); 4.637(4.9); 3.316(203.4); 2.675(2.5); 2.670(3.4); 2.666(2.7); 2.574 |
| | | | (0.4); 2.566(0.4); 2.505(374.9); 2.501(499.7); 2.497(391.8); 2.332(2.3); 2.328(3.2); |
| | | | 2.324(2.5); 2.065(0.4); 2.050(1.2); 2.035(1.7); 2.019(1.3); 2.003(0.5); 1.988(1.1); |
| | | | 1.398(16.0); 1.175(0.6); 0.866(11.3); 0.854(6.0); 0.848(5.7); 0.146(3.2); |
| | | | 0.031(0.6); 0.007(31.0); 0.000(642.1); −0.029(1.4); −0.057(0.5); −0.150(3.2) |
| I-155 | 3.40 | 3.46 | Example I-155: ¹H-NMR(400.0 MHz, d₆-DMSO): δ = |
| | | | 10.999(5.9); 8.971(1.8); 8.956(3.5); 8.940(1.7); 8.480(7.2); 8.476(7.5); 8.314(0.8); |
| | | | 7.998(7.6); 7.994(7.8); 7.838(8.5); 7.817(10.2); 7.790(2.0); 7.781(7.7); 7.759(7.5); |
| | | | 7.678(0.4); 7.656(0.4); 7.554(8.8); 7.534(7.7); 7.511(0.6); 7.495(2.5); |
| | | | 7.462(0.4); 7.458(0.4); 7.315(4.9); 7.302(0.4); 7.135(2.5); 6.927(0.4); 6.923(0.4); |
| | | | 6.284(0.4); 4.653(6.7); 4.637(6.7); 4.585(0.4); 4.569(0.4); 3.367(0.5); 3.318(198.8); |
| | | | 2.675(1.9); 2.671(2.5); 2.666(1.9); 2.555(0.7); 2.506(282.3); 2.501(370.0); |
| | | | 2.497(280.9); 2.333(1.7); 2.328(2.4); 2.324(1.8); 2.073(0.9); 2.053(1.8); |
| | | | 2.038(2.4); 2.023(2.0); 2.007(0.7); 0.872(16.0); 0.857(10.0); 0.146(1.8); 0.031(0.4); |
| | | | 0.008(16.4); 0.000(365.8); −0.008(22.6); −0.037(0.7); −0.150(1.8) |
| I-156 | 4.85 | 4.97 | Example I-156: ¹H-NMR(601.6 MHz, DMF): δ = |
| | | | 10.990(0.9); 8.669(1.9); 8.666(1.9); 8.437(0.6); 8.057(2.2); 8.054(2.2); 8.025(4.3); |
| | | | 7.996(2.5); 7.993(0.9); 7.985(0.9); 7.982(2.7); 7.613(1.6); 7.600(1.5); 3.954(0.5); |
| | | | 3.518(1.0); 3.507(1.4); 3.506(1.4); 3.494(1.0); 3.468(16.0); 3.439(0.5); |
| | | | 2.922(2.2); 2.919(4.5); 2.915(6.2); 2.912(4.4); 2.909(2.2); 2.751(2.4); 2.748(4.7); |
| | | | 2.745(6.6); 2.742(4.6); 2.738(2.3); 2.178(0.3); 2.171(0.5); 2.163(0.4); 2.014(0.3); |
| | | | 2.008(0.4); 2.002(0.3); 1.708(1.6); 1.701(2.5); 1.698(2.3); 1.682(1.1); |
| | | | 1.661(0.4); 1.653(0.4); 1.645(0.5); 1.635(0.6); 1.624(0.4); 0.970(0.9); 0.966(1.3); |
| | | | 0.958(1.3); 0.954(0.5); 0.937(0.6); 0.932(1.2); 0.927(0.7); 0.924(0.7); 0.919 |
| | | | (1.2); 0.914(0.6); 0.000(6.7) |
| I-157 | 3.26 | 3.38 | Example I-157: ¹H-NMR(400.0 MHz, d₆-DMSO): δ = |
| | | | 10.982(5.7); 8.517(7.3); 8.513(7.5); 8.404(1.6); 8.388(3.2); 8.373(1.6); 7.947(7.9); |
| | | | 7.943(8.0); 7.916(1.1); 7.909(8.8); 7.892(3.3); 7.887(10.0); 7.541(6.5); 7.521(5.9); |
| | | | 5.755(2.1); 3.412(1.4); 3.396(4.6); 3.381(4.8); 3.365(1.7); 3.321(30.0); |
| | | | 2.677(0.4); 2.673(0.6); 2.668(0.4); 2.582(3.7); 2.566(9.0); 2.549(3.6); 2.526 |
| | | | (1.4); 2.508(63.6); 2.504(83.6); 2.499(62.5); 2.335(0.4); 2.330(0.5); 2.326(0.4); |
| | | | 2.080(0.5); 2.064(1.6); 2.049(2.4); 2.034(1.8); 2.019(0.7); 1.670(1.6); 1.652(7.1); |
| | | | 1.643(7.1); 1.635(6.8); 1.617(1.6); 1.353(0.4); 1.310(0.7); 1.250(0.4); 1.230(0.7); |
| | | | 0.877(16.0); 0.865(8.4); 0.859(8.0); 0.835(0.4); 0.008(1.7); 0.000(44.0) |
| I-158 | 4.02 | 4.17 | Example I-158: ¹H-NMR(400.0 MHz, d₆-DMSO): δ = |
| | | | 11.018(2.8); 8.747(1.7); 8.727(1.7); 8.537(3.5); 7.922(3.7); 7.901(3.9); 7.879(4.4); |
| | | | 7.843(3.8); 7.823(4.7); 7.637(4.4); 7.617(3.6); 7.536(3.2); 7.515(2.9); 5.254(0.9); |
| | | | 5.236(1.3); 5.217(0.9); 4.039(0.6); 4.021(0.6); 3.319(29.8); 2.672(0.6); |
| | | | 2.502(98.6); 2.329(0.6); 2.072(0.9); 2.057(1.1); 2.041(0.9); 1.989(2.4); 1.564 |
| | | | (5.6); 1.547(5.6); 1.398(16.0); 1.194(0.6); 1.176(1.2); 1.158(0.6); 0.882(7.1); |
| | | | 0.868(4.0); 0.864(4.2); 0.000(38.2) |
| I-159 | 3.93 | 4.08 | Example I-159: ¹H-NMR(400.0 MHz, d₆-DMSO): δ = |
| | | | 10.991(1.8); 9.123(2.1); 8.545(2.2); 8.542(2.2); 7.933(2.4); 7.930(2.3); 7.915(2.6); |
| | | | 7.893(2.9); 7.765(2.6); 7.744(2.9); 7.544(2.0); 7.523(1.9); 7.389(2.9); 7.368(2.6); |
| | | | 3.319(16.4); 2.671(0.4); 2.506(44.1); 2.502(55.3); 2.498(41.0); 2.329(0.3); |
| | | | 2.064(0.5); 2.050(0.7); 2.035(0.6); 1.455(7.4); 1.398(16.0); 1.311(0.6); 0.881(4.7); |
| | | | 0.869(2.5); 0.863(2.3); 0.000(25.3) |
| I-160 | | 3.68 | Example I-160: ¹H-NMR(400.0 MHz, d₆-DMSO): δ = |
| | | | 11.030(7.7); 10.929(0.4); 8.948(2.2); 8.932(4.6); 8.916(2.2); 8.589(0.6); 8.551 |
| | | | (9.4); 8.548(10.0); 8.511(0.6); 8.266(0.5); 8.059(0.6); 8.024(10.0); 8.020(10.4); |
| | | | 7.941(4.3); 7.933(5.0); 7.838(15.6); 7.817(14.1); 7.553(12.7); 7.532(11.0); |
| | | | 4.650(9.8); 4.634(9.8); 3.469(0.4); 3.453(0.4); 3.432(0.6); 3.426(0.5); 3.398(0.9); |
| | | | 3.326(1198.6); 3.284(0.8); 3.271(0.7); 3.260(0.6); 3.237(0.4); 2.711(0.7); |
| | | | 2.675(3.1); 2.671(4.2); 2.667(3.2); 2.606(0.4); 2.541(145.4); 2.524(12.7); |
| | | | 2.506(496.4); 2.502(656.3); 2.498(502.1); 2.408(0.3); 2.367(0.7); 2.333(3.0); |
| | | | 2.328(4.1); 2.324(3.2); 2.067(0.7); 2.049(2.0); 2.044(1.7); 2.036(3.3); 2.023(2.1); 2.005 |
| | | | (0.9); 1.298(0.4); 1.259(0.6); 1.235(1.2); 1.187(0.4); 0.904(0.5); 0.879(16.0); |
| | | | 0.872(12.3); 0.860(8.9); 0.837(0.7); 0.008(1.5); 0.000(45.1) |
| I-161 | 3.74 | 3.87 | Example I-161: ¹H-NMR(400.0 MHz, d₆-DMSO): δ = |
| | | | 10.980(5.8); 8.518(7.3); 8.514(7.7); 8.348(1.5); 8.332(3.2); 8.316(1.8); 7.942(8.0); |
| | | | 7.938(8.4); 7.911(0.9); 7.904(8.8); 7.887(3.2); 7.882(10.2); 7.540(6.5); 7.519(5.8); |
| | | | 5.755(8.5); 4.022(0.3); 3.570(1.3); 3.478(5.8); 3.241(3.5); 3.224(6.5); |
| | | | 3.208(3.6); 3.188(0.3); 2.688(0.5); 2.678(1.3); 2.673(1.1); 2.658(1.2); 2.649(2.2); |
| | | | 2.640(1.3); 2.628(0.8); 2.619(1.2); 2.611(0.7); 2.526(1.3); 2.513(29.5); 2.508(60.9); |
| | | | 2.504(81.4); 2.499(61.4); 2.335(0.4); 2.331(0.5); 2.326(0.4); 2.079(0.6); |
| | | | 2.062(2.3); 2.048(4.7); 2.033(3.4); 2.017(3.6); 2.011(3.0); 1.990(1.6); 1.788(2.5); |
| | | | 1.781(2.7); 1.755(2.9); 1.748(2.8); 1.608(0.7); 1.598(0.9); 1.589(1.1); |
| | | | 1.581(1.3); 1.572(1.1); 1.562(0.9); 1.553(0.8); 1.538(1.2); 1.530(1.2); 1.506(2.7); |
| | | | 1.499(2.7); 1.475(2.7); 1.468(2.7); 1.444(1.1); 1.436(0.9); 1.398(0.5); 1.307 |
| | | | (0.3); 1.233(0.4); 1.194(0.4); 1.176(0.8); 1.159(0.4); 1.087(1.1); 1.079(1.2); |
| | | | 1.054(2.6); 1.049(2.6); 1.023(2.4); 1.017(2.4); 0.993(0.9); 0.985(0.8); 0.880(16.0); |
| | | | 0.866(9.2); 0.863(9.1); 0.839(0.4); 0.000(1.7) |
| I-162 | 3.93 | 3.88 | Example I-162: ¹H-NMR(400.0 MHz, d₆-DMSO): δ = |
| | | | 10.988(4.2); 8.995(2.4); 8.974(2.4); 8.517(5.6); 8.514(5.9); 8.314(0.8); 7.918(6.4); |
| | | | 7.914(7.0); 7.906(6.9); 7.884(7.7); 7.540(5.0); 7.520(4.5); 5.281(1.1); 5.260(2.2); |
| | | | 5.239(2.2); 5.218(1.1); 3.600(3.0); 3.577(6.1); 3.554(3.5); 3.318(75.5); |
| | | | 3.304(6.1); 3.301(5.9); 3.281(3.1); 2.676(0.9); 2.671(1.2); 2.667(0.9); 2.524(3.5); |
| | | | 2.506(146.8); 2.502(193.1); 2.498(145.2); 2.333(0.9); 2.329(1.2); 2.324(1.0); |
| | | | 2.095(0.4); 2.080(1.4); 2.065(1.8); 2.049(1.6); 2.034(0.5); 1.398(16.0); |
| | | | 0.879(12.7); 0.865(7.9); 0.146(1.1); 0.008(9.4); |
| | | | 0.000(235.8); −0.008(11.9); −0.150(1.1) |
| I-163 | 4.24 | 4.32 | Example I-163: ¹H-NMR(400.0 MHz, d₆-DMSO): δ = |
| | | | 11.032(3.9); 8.537(5.2); 8.533(5.5); 8.450(4.5); 8.314(1.0); 7.933(5.7); 7.929(5.8); |
| | | | 7.916(0.8); 7.909(6.1); 7.887(7.1); 7.545(4.5); 7.525(4.1); 3.873(5.1); 3.850(5.4); |
| | | | 3.317(213.6); 3.286(0.3); 3.031(6.5); 3.007(6.3); 2.973(0.4); 2.675(1.8); |
| | | | 2.670(2.6); 2.666(2.0); 2.563(0.5); 2.524(5.9); 2.510(150.2); 2.506(306.9); |
| | | | 2.501(407.8); 2.497(304.6); 2.333(1.9); 2.328(2.6); 2.324(2.0); 2.063(0.4); |
| | | | 2.048(1.3); 2.033(1.7); 2.017(1.4); 2.002(0.5); 1.789(16.0); 1.398(0.5); |
| | | | 0.879(11.4); 0.863(7.4); 0.146(2.3); 0.026(0.4); 0.008(18.0); |
| | | | 0.000(514.1); −0.008(24.4); −0.031(0.7); −0.150(2.4) |
| I-164 | 4.24 | 4.42 | Example I-164: ¹H-NMR(400.0 MHz, d₆-DMSO): δ = |
| | | | 10.939(1.4); 8.662(0.8); 8.641(0.9); 8.537(1.6); 8.535(1.6); 8.014(1.7); 8.011(1.7); |
| | | | 7.927(1.9); 7.906(2.2); 7.789(1.5); 7.696(0.8); 7.676(0.9); 7.553(1.5); 7.532(1.4); |
| | | | 7.468(1.1); 7.448(1.0); 5.631(0.6); 5.610(0.6); 3.318(69.7); 3.069(0.4); |
| | | | 3.055(0.3); 2.972(0.5); 2.951(0.4); 2.671(0.7); 2.595(0.4); 2.586(0.4); 2.575(0.4); |
| | | | 2.564(0.5); 2.556(0.4); 2.506(87.3); 2.502(112.7); 2.328(0.7); 2.083(0.4); |
| | | | 2.061(0.4); 2.051(0.4); 2.029(0.4); 2.002(0.4); 1.986(0.6); 1.970(0.4); 1.398(16.0); |
| | | | 0.855(2.9); 0.846(1.9); 0.835(1.5); 0.146(0.6); 0.008(4.5); 0.000(116.7); −0.150(0.6) |
| I-165 | 3.33 | 3.46 | Example I-165: ¹H-NMR(400.0 MHz, d₆-DMSO): δ = |
| | | | 10.984(6.5); 8.455(8.2); 8.453(8.2); 8.314(0.4); 7.888(9.0); 7.867(10.2); 7.633 |
| | | | (8.4); 7.631(8.4); 7.528(7.4); 7.507(6.8); 5.755(3.8); 3.634(2.8); 3.618(7.4); |
| | | | 3.602(3.0); 3.529(2.9); 3.512(7.2); 3.496(3.1); 3.319(91.4); 2.671(1.2); 2.554(0.3); |
| | | | 2.502(187.4); 2.329(1.2); 2.064(0.6); 2.048(1.8); 2.034(2.7); 2.019(2.0); 2.003(0.7); |
| | | | 1.989(0.4); 1.886(5.4); 1.870(13.9); 1.854(5.2); 1.259(0.3); 1.234(0.7); |
| | | | 0.864(16.0); 0.854(9.4); 0.846(7.9); 0.146(0.5); 0.000(93.9); −0.149(0.5) |
| I-166 | | 3.68 | Example I-166: ¹H-NMR(400.0 MHz, d₆-DMSO): δ = |
| | | | 20.009(1.4); 11.213(2.8); 8.549(3.1); 8.492(3.3); 8.459(1.3); 8.313(5.1); 8.071 |
| | | | (1.7); 8.053(1.7); 7.919(3.4); 7.897(4.1); 7.841(3.5); 7.546(2.8); 7.525(2.5); |
| | | | 6.813(3.6); 3.316(1105.6); 2.670(16.0); 2.505(1834.6); 2.501(2416.5); 2.497(1870.4); |
| | | | 2.328(14.7); 2.048(1.1); 1.235(1.7); 0.856(5.4); 0.839(3.3); 0.146(7.1); |
| | | | 0.000(1354.9); −0.150(6.1) |
| I-167 | 4.89 | 5.11 | Example I-167: ¹H-NMR(400.0 MHz, d₆-DMSO): δ = |
| | | | 10.978(3.4); 8.524(4.0); 8.521(4.2); 8.316(0.5); 8.293(0.9); 8.278(1.9); 8.262(1.0); |
| | | | 7.946(4.2); 7.943(4.4); 7.905(4.7); 7.883(5.4); 7.540(3.9); 7.520(3.5); 3.321(12.5); |
| | | | 3.238(2.2); 3.221(4.2); 3.205(2.3); 2.504(44.2); 2.061(1.0); 2.046(1.4); |
| | | | 2.030(1.1); 2.015(0.4); 1.749(1.8); 1.717(3.7); 1.688(2.0); 1.642(1.0); 1.622 |
| | | | (0.8); 1.576(0.5); 1.567(0.6); 1.558(0.7); 1.549(0.8); 1.541(0.7); 1.531(0.6); |
| | | | 1.523(0.5); 1.514(0.3); 1.398(16.0); 1.262(0.3); 1.231(1.0); 1.201(1.9); 1.181(1.9); |
| | | | 1.138(0.5); 1.014(0.8); 0.984(1.8); 0.956(1.5); 0.933(0.5); 0.922(0.4); 0.881 |
| | | | (9.1); 0.868(5.3); 0.000(21.0) |
| I-168 | 4.57 | 4.74 | Example I-168: ¹H-NMR(400.0 MHz, d₆-DMSO): δ = |
| | | | 11.006(2.6); 8.524(3.4); 8.520(3.6); 8.314(1.2); 8.103(1.4); 8.082(1.4); 7.944(3.6); |
| | | | 7.940(3.8); 7.903(4.0); 7.881(4.6); 7.539(3.0); 7.518(2.7); 3.820(0.5); 3.809(0.5); |
| | | | 3.799(0.6); 3.317(71.3); 3.294(0.5); 2.675(0.7); 2.671(1.0); 2.666(0.8); |
| | | | 2.524(2.8); 2.506(114.0); 2.502(151.1); 2.497(114.4); 2.333(0.7); 2.328(1.0); |
| | | | 2.324(0.7); 2.051(0.9); 2.036(1.1); 2.021(1.0); 1.909(1.5); 1.885(1.2); 1.760(1.0); |
| | | | 1.750(0.9); 1.728(1.5); 1.632(0.6); 1.601(0.6); 1.420(0.4); 1.398(16.0); 1.359(2.1); |
| | | | 1.332(2.6); 1.308(1.0); 1.302(1.0); 1.272(0.3); 1.245(0.3); 1.223(0.6); |
| | | | 1.193(0.5); 0.879(7.5); 0.863(5.1); 0.146(0.4); 0.008(3.3); 0.000(87.5); −0.150(0.4) |
| I-169 | 4.22 | 4.35 | Example I-169: ¹H-NMR(400.0 MHz, d₆-DMSO): δ = |
| | | | 10.986(4.6); 8.520(5.5); 8.516(6.0); 8.314(1.4); 8.171(2.5); 8.152(2.5); 7.935(5.7); |
| | | | 7.931(6.2); 7.903(6.6); 7.882(7.7); 7.539(5.2); 7.518(4.7); 4.280(1.0); 4.262(1.8); |
| | | | 4.245(1.8); 4.227(0.9); 3.317(104.9); 3.293(0.8); 2.670(1.4); 2.666(1.2); |
| | | | 2.506(167.2); 2.502(223.5); 2.497(176.7); 2.328(1.4); 2.062(0.4); 2.047(1.5); |
| | | | 2.032(2.0); 2.016(2.2); 2.002(1.9); 1.988(2.2); 1.971(2.2); 1.957(1.8); 1.940 |
| | | | (0.9); 1.761(0.4); 1.721(2.5); 1.705(2.0); 1.682(0.5); 1.649(0.7); 1.624(1.9); |
| | | | 1.612(1.8); 1.606(2.4); 1.595(2.3); 1.585(1.4); 1.577(1.2); 1.566(1.4); 1.546(1.8); |
| | | | 1.530(2.0); 1.516(2.0); 1.501(1.4); 1.481(0.5); 1.398(16.0); 0.878(12.8); |
| | | | 0.863(9.0); 0.146(0.6); 0.007(4.3); 0.000(113.4); −0.150(0.6) |
| I-170 | 4.55 | 4.68 | Example I-170: ¹H-NMR(400.0 MHz, d₆-DMSO): δ = |
| | | | 10.981(5.7); 8.526(7.2); 8.523(7.3); 8.314(1.7); 8.298(3.3); 8.283(1.7); 7.954(7.8); |
| | | | 7.950(7.8); 7.914(1.3); 7.907(8.7); 7.890(3.5); 7.885(9.9); 7.540(6.4); 7.520(5.8); |
| | | | 5.756(0.5); 3.326(30.2); 3.319(5.2); 3.302(6.8); 3.285(4.4); 2.510(37.6); |
| | | | 2.506(48.3); 2.501(35.8); 2.185(0.8); 2.167(2.0); 2.148(2.7); 2.130(2.1); 2.111(0.9); |
| | | | 2.078(0.6); 2.062(1.7); 2.047(2.4); 2.032(1.9); 2.016(0.7); 1.767(0.9); |
| | | | 1.750(1.9); 1.738(2.5); 1.719(2.8); 1.707(2.1); 1.691(1.3); 1.667(0.5); 1.659(0.6); |
| | | | 1.640(1.7); 1.633(2.1); 1.620(3.1); 1.603(2.7); 1.581(1.3); 1.569(1.7); 1.545 |
| | | | (2.6); 1.536(2.3); 1.529(2.9); 1.518(2.2); 1.500(1.0); 1.485(0.4); 1.479(0.4); |
| | | | 1.397(2.0); 1.353(0.7); 1.335(0.4); 1.313(1.1); 1.297(2.5); 1.279(2.8); 1.266(2.6); |
| | | | 1.250(2.4); 1.230(1.3); 0.884(16.0); 0.871(8.6); 0.866(8.1); 0.008(0.7); 0.000 |
| | | | (11.7) |
| I-171 | 3.82 | 3.96 | Example I-171: ¹H-NMR(400.0 MHz, d₆-DMSO): δ = |
| | | | 10.937(3.7); 8.945(1.0); 8.929(2.2); 8.913(1.0); 8.511(4.5); 8.507(4.6); 8.314(2.2); |
| | | | 7.965(5.0); 7.961(4.9); 7.869(5.4); 7.847(6.4); 7.837(5.9); 7.817(6.4); 7.555(5.6); |
| | | | 7.534(4.9); 7.474(5.0); 7.452(4.5); 4.655(4.2); 4.639(4.3); 3.317(273.6); |
| | | | 3.295(0.9); 2.675(2.4); 2.671(3.2); 2.666(2.4); 2.576(0.5); 2.506(379.3); 2.501(491.7); |
| | | | 2.497(367.5); 2.444(0.4); 2.333(2.3); 2.328(3.1); 2.324(2.4); 2.068(0.4); |
| | | | 2.050(1.0); 2.037(1.5); 2.024(1.0); 2.005(0.5); 1.988(0.6); 1.398(16.0); 0.866(7.3); |
| | | | 0.860(5.6); 0.846(3.9); 0.824(0.4); 0.008(2.7); 0.000(54.6) |
| I-172 | 4.57 | 4.27 | Example I-172: ¹H-NMR(400.0 MHz, d₆-DMSO): δ = |
| | | | 10.976(6.1); 8.959(1.7); 8.944(3.6); 8.928(1.7); 8.497(7.2); 8.494(7.5); 8.479(0.4); |
| | | | 8.314(0.4); 7.993(7.7); 7.989(7.9); 7.933(6.5); 7.928(6.9); 7.838(8.7); 7.817(10.3); |
| | | | 7.754(2.8); 7.749(2.7); 7.732(4.1); 7.727(4.2); 7.654(7.5); 7.633(5.0); |
| | | | 7.556(8.8); 7.535(7.8); 5.754(11.4); 4.655(6.8); 4.640(6.9); 4.039(0.5); 4.021 |
| | | | (0.5); 3.321(66.3); 2.672(0.8); 2.667(0.6); 2.507(99.5); 2.503(129.0); 2.498(97.1); |
| | | | 2.330(0.9); 2.325(0.6); 2.070(0.6); 2.054(1.7); 2.039(2.4); 2.024(2.0); 2.008(0.7); |
| | | | 1.989(2.3); 1.398(2.2); 1.232(0.4); 1.194(0.6); 1.176(1.2); 1.158(0.6); 0.870(16.0); |
| | | | 0.857(9.2); 0.853(8.8); 0.146(0.8); 0.007(9.5); 0.000(170.2); −0.008(9.5); −0.150(0.8) |
| I-173 | 4.31 | 3.98 | Example I-173: ¹H-NMR(400.0 MHz, d₆-DMSO): δ = |
| | | | 11.015(1.6); 8.971(0.5); 8.955(1.0); 8.939(0.5); 8.572(1.9); 8.569(2.0); 8.314(0.3); |
| | | | 8.082(1.6); 8.059(3.1); 8.021(2.3); 8.017(2.7); 8.011(2.2); 7.989(1.3); 7.838(2.3); |
| | | | 7.818(2.7); 7.558(2.4); 7.537(2.1); 4.660(1.9); 4.645(1.9); 3.335(1.6); |
| | | | 3.318(75.6); 2.675(0.8); 2.671(1.0); 2.506(119.7); 2.502(155.9); 2.497(120.3); |
| | | | 2.328(1.0); 2.060(0.4); 2.046(0.7); 2.030(0.5); 1.988(0.9); 1.398(16.0); 1.175(0.4); |
| | | | 0.876(3.8); 0.866(2.4); 0.856(1.9); 0.146(0.8); 0.036(0.4); 0.008(9.5); |
| | | | 0.000(175.0); −0.150(0.9) |
| I-174 | 3.22 | 3.25 | Example I-174: ¹H-NMR(400.0 MHz, d₆-DMSO): δ = |
| | | | 11.012(8.2); 9.164(7.4); 8.994(11.4); 8.991(11.9); 8.970(2.2); 8.955(4.6); |
| | | | 8.939(2.2); 8.409(3.0); 8.398(12.9); 8.394(13.3); 8.389(5.7); 8.383(5.3); 8.339(7.9); |
| | | | 8.318(4.4); 7.842(13.3); 7.821(15.9); 7.808(0.6); 7.565(13.7); 7.544(11.9); |
| | | | 7.514(0.4); 4.671(10.3); 4.655(10.3); 4.626(0.3); 4.613(0.4); 3.410(12.1); 3.356(18.0); |
| | | | 2.676(0.9); 2.672(1.3); 2.668(1.0); 2.525(3.0); 2.507(166.3); 2.503(222.3); |
| | | | 2.499(162.9); 2.334(0.9); 2.330(1.3); 2.325(1.0); 2.081(0.8); 2.068(1.8); 2.063(2.1); |
| | | | 2.058(1.7); 2.051(3.6); 2.038(2.2); 2.033(2.1); 2.020(1.0); 1.070(1.8); |
| | | | 0.912(0.6); 0.888(11.5); 0.879(16.0); 0.860(8.6); 0.840(1.0); 0.824(0.3); 0.008 |
| | | | (0.7); 0.000(21.9); −0.008(0.8) |
| I-175 | 3.17 | 3.25 | Example I-175: ¹H-NMR(400.0 MHz, d₆-DMSO): δ = |
| | | | 11.009(1.2); 9.441(1.5); 8.547(1.6); 8.544(1.5); 8.313(2.1); 7.917(3.4); 7.895(2.1); |
| | | | 7.546(1.4); 7.526(1.3); 4.955(1.9); 4.938(2.2); 4.771(2.1); 4.754(1.8); 3.740(7.4); |
| | | | 3.314(60.6); 3.290(1.1); 2.675(1.1); 2.670(1.5); 2.666(1.1); 2.506(172.3); |
| | | | 2.501(219.7); 2.497(165.8); 2.332(1.1); 2.328(1.4); 2.323(1.1); 2.110(0.3); |
| | | | 2.094(0.5); 2.086(0.7); 1.398(16.0); 0.882(3.1); 0.871(1.8); 0.863(1.6); 0.000(6.1) |
| I-176 | 3.83 | | Example I-176: ¹H-NMR(400.0 MHz, d₆-DMSO): δ = |
| | | | 10.969(6.1); 8.550(1.7); 8.535(3.5); 8.519(9.0); 8.517(8.4); 8.313(3.1); 7.961(7.8); |
| | | | 7.958(7.5); 7.909(8.5); 7.887(9.5); 7.545(6.7); 7.524(6.2); 5.310(0.4); 4.056(0.6); |
| | | | 4.039(1.8); 4.021(1.9); 4.003(0.7); 3.638(2.1); 3.621(5.6); 3.605(5.7); |
| | | | 3.588(2.3); 3.394(0.4); 3.378(0.5); 3.358(0.7); 3.317(521.6); 3.294(3.1); 2.671 |
| | | | (3.2); 2.667(2.9); 2.648(1.4); 2.637(2.1); 2.619(3.6); 2.608(2.5); 2.603(2.3); |
| | | | 2.591(3.9); 2.574(2.3); 2.562(1.8); 2.544(1.6); 2.506(365.5); 2.502(466.1); |
| | | | 2.498(367.2); 2.329(3.0); 2.099(0.5); 2.083(1.7); 2.067(2.4); 2.052(1.9); |
| | | | 2.036(0.6); 1.988(7.7); 1.399(9.5); 1.308(0.4); 1.193(2.0); 1.175(4.1); 1.158(2.0); |
| | | | 0.876(16.0); 0.863(9.1); 0.859(8.7); 0.146(2.3); 0.029(0.4); |
| | | | 0.000(468.9); −0.032(0.9); −0.045(0.4); −0.052(0.3); −0.149(2.3) |
| I-177 | 3.26 | 3.29 | Example I-177: ¹H-NMR(400.0 MHz, d₆-DMSO): δ = |
| | | | 11.066(6.0); 8.541(7.9); 8.308(1.8); 8.296(3.3); 8.282(1.7); 7.969(8.4); 7.908(7.7); |
| | | | 7.886(9.0); 7.543(7.2); 7.522(6.4); 5.755(2.8); 3.550(4.2); 3.537(6.1); 3.525(5.1); |
| | | | 3.515(8.6); 3.504(7.1); 3.324(65.0); 3.305(35.2); 2.672(0.7); 2.503(106.8); |
| | | | 2.330(0.7); 2.083(0.6); 2.067(1.8); 2.052(2.4); 2.036(2.0); 2.021(0.6); 1.306(0.7); |
| | | | 0.878(16.0); 0.865(9.5); 0.000(3.2) |
| I-178 | | 3.81 | Example I-178: ¹H-NMR(400.0 MHz, d₆-DMSO): δ = |
| | | | 10.986(6.2); 8.957(2.0); 8.941(3.9); 8.925(1.8); 8.559(7.8); 8.556(7.9); 8.313(1.9); |
| | | | 8.002(8.3); 7.999(8.4); 7.910(7.1); 7.889(11.9); 7.834(16.0); 7.817(14.0); |
| | | | 7.558(9.8); 7.537(8.4); 6.900(0.7); 6.891(1.3); 6.884(0.7); 6.769(1.5); 6.761(2.7); |
| | | | 6.752(1.5); 6.637(0.7); 6.630(1.4); 6.621(0.7); 4.658(7.5); 4.643(7.5); 4.056 |
| | | | (0.4); 4.038(1.0); 4.020(1.0); 4.003(0.4); 3.316(276.4); 2.691(0.6); 2.675(4.0); |
| | | | 2.670(5.2); 2.666(4.1); 2.637(0.5); 2.592(0.8); 2.506(591.4); 2.501(756.2); |
| | | | 2.497(591.7); 2.328(4.7); 2.073(0.7); 2.057(1.8); 2.043(2.6); 2.027(1.9); 2.012(0.7); |
| | | | 1.988(4.0); 1.398(2.8); 1.236(0.5); 1.193(1.1); 1.175(2.1); 1.157(1.0); 1.069 |
| | | | (0.5); 0.871(15.9); 0.860(9.5); 0.853(8.1); 0.146(0.6); 0.000(120.6); −0.150(0.6) |
| I-179 | 2.47 | 2.53 | Example I-179: ¹H-NMR(400.0 MHz, d₆-DMSO): δ = |
| | | | 11.003(6.7); 8.971(1.7); 8.956(3.5); 8.940(1.7); 8.579(7.6); 8.576(8.1); 8.314(0.4); |
| | | | 8.070(7.8); 8.049(11.3); 8.030(7.9); 8.027(8.4); 7.949(10.3); 7.928(7.6); |
| | | | 7.841(9.1); 7.820(10.7); 7.645(0.7); 7.627(1.2); 7.615(1.0); 7.597(1.0); 7.561(10.0); |
| | | | 7.540(8.6); 7.174(2.4); 7.039(4.7); 6.905(2.8); 5.754(12.5); 4.662(7.3); 4.646(7.3); |
| | | | 3.324(195.7); 3.322(191.7); 2.671(1.5); 2.667(1.2); 2.506(179.6); 2.502(236.9); |
| | | | 2.498(188.4); 2.329(1.5); 2.077(0.6); 2.062(1.7); 2.047(2.5); 2.031(1.9); |
| | | | 2.016(0.7); 1.235(1.3); 0.875(16.0); 0.863(8.9); 0.857(8.2); 0.833(0.5); 0.000(8.3) |
| I-180 | 3.46 | 3.55 | Example I-180: ¹H-NMR(400.0 MHz, d₆-DMSO): δ = |
| | | | 10.966(8.0); 8.952(2.1); 8.936(4.6); 8.920(2.2); 8.545(9.8); 8.541(10.2); 8.314 |
| | | | (6.0); 7.992(10.8); 7.988(11.0); 7.972(0.4); 7.866(10.1); 7.845(15.7); 7.838(13.1); |
| | | | 7.817(14.2); 7.749(14.3); 7.728(10.3); 7.717(3.9); 7.578(8.6); 7.557(12.1); |
| | | | 7.536(10.6); 7.439(4.0); 5.754(2.4); 4.657(8.5); 4.641(8.6); 3.361(0.3); 3.344(0.7); |
| | | | 3.317(326.9); 3.293(3.0); 2.675(2.4); 2.670(3.3); 2.666(2.5); 2.554(0.9); |
| | | | 2.548(1.1); 2.524(8.5); 2.510(177.2); 2.506(359.6); 2.501(490.4); 2.497(375.2); |
| | | | 2.492(189.4); 2.333(2.3); 2.328(3.2); 2.324(2.4); 2.073(2.4); 2.054(1.8); |
| | | | 2.040(3.0); 2.027(1.9); 2.024(1.9); 2.009(0.8); 1.233(0.6); 0.869(16.0); |
| | | | 0.860(10.0); 0.849(8.1); 0.827(0.5); 0.008(2.0); 0.000(56.3); −0.008(2.2) |
| I-181 | 5.43 | 5.54 | Example I-181: ¹H-NMR(400.0 MHz, d₆-DMSO): δ = |
| | | | 12.365(3.3); 11.187(2.2); 8.893(2.4); 8.503(8.5); 8.499(7.8); 8.313(0.3); 7.935 |
| | | | (8.5); 7.914(9.7); 7.842(9.0); 7.821(10.4); 7.583(16.0); 7.563(14.2); 5.753(1.3); |
| | | | 5.140(7.2); 5.124(7.2); 3.790(0.6); 3.770(0.4); 3.461(0.5); 3.449(0.5); 3.424(0.9); |
| | | | 3.346(469.5); 3.339(332.6); 3.252(0.6); 2.677(1.1); 2.672(1.4); 2.668(1.1); |
| | | | 2.549(1.7); 2.525(4.4); 2.508(181.1); 2.503(227.4); 2.499(170.6); 2.334(1.2); |
| | | | 2.330(1.5); 2.326(1.1); 1.352(0.4); 1.298(0.5); 1.259(0.8); 1.234(2.0); 1.188(4.1); |
| | | | 1.180(6.2); 1.171(4.8); 1.079(0.4); 1.056(3.4); 1.047(4.0); 1.038(4.2); 1.029(3.1); |
| | | | 0.862(0.5); 0.854(0.5); 0.843(0.5); 0.825(0.3); 0.000(4.9) |
| I-182 | 4.07 | 4.24 | Example I-182: ¹H-NMR(400.0 MHz, d₆-DMSO): δ = |
| | | | 11.005(2.8); 8.968(0.7); 8.953(1.5); 8.937(0.7); 8.582(3.6); 8.579(3.7); 8.313(0.4); |
| | | | 8.036(3.2); 8.030(4.7); 8.026(4.4); 8.015(4.1); 7.883(3.9); 7.862(3.1); 7.840(4.3); |
| | | | 7.819(5.2); 7.560(4.4); 7.540(3.9); 4.662(3.3); 4.647(3.3); 3.321(62.7); |
| | | | 3.318(62.1); 2.675(0.5); 2.671(0.7); 2.667(0.5); 2.506(79.9); 2.502(106.4); |
| | | | 2.497(81.0); 2.493(42.7); 2.333(0.5); 2.329(0.6); 2.324(0.5); 2.061(0.7); 2.054(0.6); |
| | | | 2.047(1.2); 2.030(0.8); 2.016(0.3); 1.988(0.9); 1.398(16.0); 1.175(0.5); 0.875(6.7); |
| | | | 0.865(3.9); 0.856(3.3); 0.008(1.7); 0.000(45.7); −0.008(2.1) |
| I-183 | 3.18 | 3.22 | Example I-183: ¹H-NMR(400.0 MHz, d₆-DMSO): δ = |
| | | | 11.069(5.3); 8.479(8.5); 8.475(8.5); 8.314(0.4); 7.897(10.6); 7.893(3.6); 7.880 |
| | | | (3.9); 7.875(12.0); 7.868(1.3); 7.580(4.5); 7.531(7.7); 7.510(6.9); 4.056(0.4); |
| | | | 4.039(1.1); 4.021(1.2); 4.003(0.4); 3.693(16.0); 3.318(146.2); 3.295(37.1); 2.676(0.9); |
| | | | 2.671(1.2); 2.666(0.9); 2.524(3.4); 2.511(75.4); 2.507(150.0); 2.502(196.1); |
| | | | 2.498(141.8); 2.493(68.8); 2.333(0.9); 2.329(1.2); 2.324(0.8); 2.077(0.7); |
| | | | 2.064(1.5); 2.060(1.7); 2.054(1.3); 2.046(2.8); 2.039(1.2); 2.033(1.8); 2.030(1.8); |
| | | | 2.016(0.7); 1.989(4.9); 1.398(0.9); 1.299(0.3); 1.259(0.6); 1.250(0.5); 1.235 |
| | | | (1.1); 1.193(1.3); 1.175(2.7); 1.158(1.3); 1.046(0.4); 1.030(0.4); 0.862(14.5); |
| | | | 0.853(9.4); 0.842(7.3); 0.000(0.9) |
| I-184 | | 4.23 | Example I-184: ¹H-NMR(400.0 MHz, d₆-DMSO): δ = |
| | | | 10.987(5.3); 8.525(5.8); 8.519(9.1); 8.515(8.3); 8.313(6.3); 7.934(8.3); 7.930(8.2); |
| | | | 7.904(9.0); 7.900(3.1); 7.888(3.2); 7.882(10.3); 7.540(6.2); 7.520(5.6); 3.409(0.5); |
| | | | 3.322(729.3); 2.942(2.1); 2.675(3.7); 2.671(4.9); 2.666(3.6); 2.524(15.2); |
| | | | 2.511(308.0); 2.506(629.4); 2.502(833.9); 2.497(605.2); 2.493(295.2); 2.333(3.6); |
| | | | 2.328(4.9); 2.324(3.6); 2.050(0.6); 2.035(1.8); 2.019(2.3); 2.004(2.1); |
| | | | 1.989(0.7); 1.488(0.6); 1.475(1.3); 1.467(1.4); 1.455(2.7); 1.443(1.5); 1.435(1.5); |
| | | | 1.422(0.8); 1.236(0.4); 0.875(16.0); 0.860(11.3); 0.768(0.3); 0.757(1.9); 0.739(6.7); |
| | | | 0.728(3.2); 0.696(0.6); 0.690(0.7); 0.659(3.3); 0.647(6.5); 0.628(1.9); |
| | | | 0.416(1.4); 0.404(4.5); 0.400(4.9); 0.390(2.5); 0.384(4.6); 0.379(4.7); 0.369(1.8); |
| | | | 0.222(1.8); 0.208(5.9); 0.199(5.1); 0.195(5.5); 0.184(1.5); 0.146(1.3); 0.008(10.7); |
| | | | 0.000(295.9); −0.009(10.7); −0.150(1.3) |
| I-185 | 4.14 | 4.29 | Example I-185: ¹H-NMR(400.0 MHz, d₆-DMSO): δ = |
| | | | 11.044(6.3); 8.548(8.1); 8.544(8.8); 8.367(1.6); 8.351(3.4); 8.335(1.7); 8.313(0.8); |
| | | | 8.002(8.7); 7.998(9.2); 7.982(2.2); 7.974(1.6); 7.960(1.6); 7.954(1.9); 7.921(1.3); |
| | | | 7.901(2.8); 7.884(2.5); 7.842(4.3); 7.821(2.5); 5.754(11.4); 3.320(243.9); |
| | | | 3.243(3.8); 3.227(7.0); 3.211(3.9); 2.680(1.7); 2.676(1.9); 2.671(2.5); 2.660(1.5); |
| | | | 2.650(2.4); 2.641(1.4); 2.629(0.8); 2.620(1.3); 2.611(0.7); 2.524(3.9); |
| | | | 2.511(101.5); 2.507(213.5); 2.502(290.1); 2.498(220.5); 2.494(114.6); 2.333(1.2); |
| | | | 2.329(1.7); 2.324(1.3); 2.083(0.6); 2.066(2.4); 2.051(5.6); 2.042(3.9); 2.036(4.3); |
| | | | 2.017(3.6); 2.009(3.2); 1.788(2.7); 1.781(3.0); 1.755(3.1); 1.747(3.1); 1.620(0.5); |
| | | | 1.611(0.8); 1.601(0.9); 1.592(1.2); 1.583(1.4); 1.575(1.1); 1.565(1.0); |
| | | | 1.556(0.9); 1.536(1.3); 1.529(1.3); 1.504(3.0); 1.497(3.0); 1.473(3.0); 1.466(2.9); |
| | | | 1.442(1.2); 1.435(1.0); 1.234(0.4); 1.087(1.1); 1.079(1.3); 1.055(2.9); 1.049(2.9); |
| | | | 1.019(2.6); 0.994(0.9); 0.985(0.8); 0.885(15.7); 0.870(16.0); 0.146(0.7); |
| | | | 0.008(5.4); 0.000(164.1); −0.150(0.7) |
| I-186 | 3.00 | 3.17 | Example I-186: ¹H-NMR(400.0 MHz, d₆-DMSO): δ = |
| | | | 10.994(0.9); 10.971(6.1); 8.900(0.5); 8.878(1.8); 8.863(3.6); 8.847(1.7); 8.531 |
| | | | (8.4); 7.986(7.7); 7.965(0.4); 7.918(8.7); 7.896(9.7); 7.815(8.6); 7.794(9.1); |
| | | | 7.752(0.4); 7.733(0.5); 7.607(0.3); 7.587(0.7); 7.575(0.8); 7.547(16.0); 7.526(14.1); |
| | | | 7.377(1.6); 7.337(13.8); 5.755(0.4); 4.641(7.2); 4.625(6.5); 3.320(63.6); |
| | | | 2.671(0.9); 2.502(145.4); 2.329(0.8); 2.059(0.7); 2.042(1.8); 2.029(2.6); 2.014(1.8); |
| | | | 1.998(0.7); 1.303(0.4); 1.259(0.4); 1.233(0.9); 1.142(0.7); 0.867(14.7); 0.858(9.7); |
| | | | 0.847(7.3); 0.000(10.1) |
| I-187 | 3.00 | 3.09 | Example I-187: ¹H-NMR(400.0 MHz, d₆-DMSO): δ = |
| | | | 11.010(5.5); 8.740(3.3); 8.720(3.2); 8.527(7.6); 8.523(7.8); 8.442(0.3); 8.313(2.6); |
| | | | 8.085(0.5); 7.953(8.6); 7.949(8.7); 7.915(9.2); 7.910(3.2); 7.898(3.3); 7.893(10.5); |
| | | | 7.544(6.3); 7.524(5.8); 4.814(0.3); 4.794(1.1); 4.774(1.8); 4.756(1.8); |
| | | | 4.737(1.1); 4.716(0.3); 3.563(2.0); 3.543(2.1); 3.530(2.4); 3.510(2.3); 3.404(1.0); |
| | | | 3.395(1.2); 3.384(1.3); 3.372(2.4); 3.361(2.2); 3.350(2.6); 3.318(508.4); |
| | | | 3.273(1.6); 3.253(1.8); 3.250(1.7); 3.240(1.0); 3.230(1.5); 3.221(1.0); 3.216(1.2); |
| | | | 3.192(2.4); 3.170(2.2); 3.157(1.9); 3.138(1.8); 2.943(1.2); 2.675(3.9); 2.670(5.3); |
| | | | 2.666(3.9); 2.614(0.4); 2.524(14.5); 2.510(317.7); 2.506(661.7); 2.501(882.7); |
| | | | 2.497(641.7); 2.492(309.4); 2.333(3.6); 2.328(5.1); 2.324(3.7); 2.288(0.8); |
| | | | 2.266(1.4); 2.255(0.7); 2.243(1.5); 2.232(1.3); 2.221(0.8); 2.210(1.1); 2.188 |
| | | | (0.4); 2.099(0.6); 2.084(1.6); 2.069(2.2); 2.053(1.8); 2.037(0.6); 1.311(2.2); |
| | | | 0.874(16.0); 0.861(8.4); 0.857(8.3); 0.146(1.2); 0.008(10.0); |
| | | | 0.000(309.8); −0.009(10.4); −0.150(1.2) |
| I-188 | 3.57 | 3.70 | Example I-188: ¹H-NMR(400.0 MHz, d₆-DMSO): δ = |
| | | | 11.092(9.1); 9.085(6.7); 9.081(6.3); 9.078(6.9); 8.963(2.4); 8.947(5.2); 8.931(2.5); |
| | | | 8.784(11.4); 8.779(11.8); 8.314(0.8); 8.213(11.9); 8.208(11.9); 7.842(13.6); |
| | | | 7.821(16.0); 7.561(13.5); 7.540(11.8); 7.162(8.7); 7.155(8.8); 4.662(10.2); |
| | | | 4.646(10.2); 3.320(172.1); 2.945(1.7); 2.892(0.4); 2.785(1.3); 2.732(0.3); 2.676(0.9); |
| | | | 2.672(1.3); 2.667(1.0); 2.525(3.5); 2.512(74.7); 2.507(152.9); 2.503(203.1); |
| | | | 2.498(149.5); 2.494(74.3); 2.334(0.9); 2.330(1.2); 2.325(0.9); 2.075(7.9); |
| | | | 2.062(1.9); 2.057(2.2); 2.052(1.8); 2.045(3.7); 2.032(2.2); 2.027(2.1); 2.013(1.0); |
| | | | 1.958(1.4); 0.917(0.6); 0.894(11.6); 0.885(15.7); 0.866(8.6); 0.845(0.8); 0.146(0.9); |
| | | | 0.008(7.5); 0.000(195.9); −0.008(8.2); −0.150(0.9) |
| I-189 | 3.14 | 3.25 | Example I-189: ¹H-NMR(601.6 MHz, d₆-DMSO): δ = |
| | | | 10.964(1.6); 9.612(0.8); 9.069(0.8); 8.761(1.9); 8.522(1.8); 8.520(1.8); 8.311(0.3); |
| | | | 7.954(2.6); 7.951(2.6); 7.901(2.7); 7.897(0.9); 7.890(0.9); 7.886(3.1); 7.545(1.5); |
| | | | 7.532(1.4); 3.310(41.8); 2.613(0.3); 2.522(0.5); 2.519(0.7); 2.516(0.6); |
| | | | 2.507(15.6); 2.504(34.1); 2.501(47.4); 2.498(33.5); 2.495(15.0); 2.385(0.3); |
| | | | 2.072(16.0); 2.050(0.4); 2.040(0.6); 2.030(0.4); 1.903(0.7); 1.896(1.7); 1.890(1.8); |
| | | | 1.884(0.7); 1.259(0.7); 1.252(1.8); 1.246(1.8); 1.239(0.7); 0.872(4.5); 0.863(2.1); |
| | | | 0.860(1.9); 0.096(0.4); 0.005(3.3); 0.000(124.6); −0.006(3.7); −0.100(0.5) |
| I-190 | | 4.09 | Example I-190: ¹H-NMR(400.0 MHz, d₆-DMSO): δ = |
| | | | 10.963(6.6); 8.951(1.8); 8.935(3.9); 8.920(1.8); 8.533(9.0); 8.529(9.6); 8.313(9.3); |
| | | | 7.985(10.2); 7.981(10.6); 7.932(1.2); 7.925(10.8); 7.920(3.7); 7.908(3.7); |
| | | | 7.903(12.6); 7.895(1.6); 7.838(11.0); 7.822(4.1); 7.818(13.2); 7.643(0.3); 7.631(0.4); |
| | | | 7.627(0.7); 7.614(0.5); 7.597(0.6); 7.573(0.5); 7.558(11.1); 7.537(14.9); |
| | | | 7.518(7.3); 4.657(7.5); 4.641(7.6); 3.316(336.3); 3.292(3.7); 2.680(1.0); 2.675(2.0); |
| | | | 2.670(3.0); 2.666(2.1); 2.661(1.0); 2.524(6.7); 2.519(10.1); 2.510(166.4); |
| | | | 2.506(354.1); 2.501(477.0); 2.497(345.1); 2.492(165.2); 2.438(0.4); 2.337(1.0); |
| | | | 2.333(2.1); 2.328(3.0); 2.323(2.2); 2.071(0.7); 2.058(1.6); 2.054(1.7); 2.047(1.3); |
| | | | 2.040(2.9); 2.024(1.9); 2.010(0.8); 1.398(1.7); 1.350(0.4); 1.234(0.5); |
| | | | 0.870(16.0); 0.859(8.9); 0.850(7.6); 0.827(0.5); 0.146(1.6); 0.008(12.2); 0.000 |
| | | | (397.9); −0.009(13.7); −0.150(1.7) |
| I-191 | | 4.38 | Example I-191: ¹H-NMR(400.0 MHz, d₆-DMSO): δ = |
| | | | 10.966(6.9); 8.952(1.8); 8.936(3.9); 8.920(1.8); 8.537(9.6); 8.533(9.9); 8.313(7.5); |
| | | | 7.987(10.7); 7.983(10.7); 7.933(1.2); 7.926(11.7); 7.921(3.9); 7.909(4.0); |
| | | | 7.904(13.6); 7.896(1.4); 7.838(11.5); 7.822(4.2); 7.818(13.8); 7.644(0.6); |
| | | | 7.627(1.1); 7.615(0.8); 7.597(1.0); 7.573(0.9); 7.557(13.1); 7.551(12.5); |
| | | | 7.536(11.9); 7.529(9.6); 7.280(0.4); 7.258(0.4); 4.657(7.8); 4.642(7.9); 3.316(443.7); |
| | | | 3.292(2.9); 2.679(1.5); 2.675(3.2); 2.670(4.5); 2.666(3.2); 2.661(1.5); 2.524(10.7); |
| | | | 2.519(16.3); 2.510(252.7); 2.506(530.2); 2.501(712.3); 2.497(513.7); 2.492(245.6); |
| | | | 2.337(1.4); 2.332(3.0); 2.328(4.3); 2.323(3.1); 2.319(1.5); 2.086(1.4); 2.071(0.7); |
| | | | 2.054(1.8); 2.040(3.0); 2.024(1.9); 2.009(0.9); 1.651(0.5); 1.398(1.1); |
| | | | 1.312(0.3); 1.234(0.7); 1.127(0.4); 0.870(16.0); 0.860(9.2); 0.850(7.7); 0.146 |
| | | | (2.5); 0.008(18.0); 0.000(555.0); −0.009(18.3); −0.150(2.4) |
| I-192 | 3.40 | 3.42 | Example I-192: ¹H-NMR(400.0 MHz, d₆-DMSO): δ = |
| | | | 10.962(5.4); 8.955(1.4); 8.940(3.0); 8.925(1.4); 8.557(6.6); 8.553(6.9); 8.313(9.2); |
| | | | 8.002(7.4); 7.998(7.4); 7.893(5.6); 7.873(7.5); 7.838(8.2); 7.818(9.7); 7.743(7.4); |
| | | | 7.722(5.6); 7.558(8.2); 7.538(7.2); 4.659(5.9); 4.643(5.9); 3.411(0.4); |
| | | | 3.316(892.8); 3.292(4.7); 2.675(4.2); 2.670(5.8); 2.666(4.3); 2.619(0.4); 2.608 |
| | | | (0.5); 2.523(15.0); 2.510(341.8); 2.506(704.1); 2.501(938.8); 2.497(685.5); |
| | | | 2.492(336.2); 2.332(4.0); 2.328(5.5); 2.323(4.1); 2.086(2.3); 2.061(7.6); 2.042(2.3); |
| | | | 2.024(1.7); 2.014(16.0); 1.966(7.9); 1.398(1.6); 1.358(0.4); 1.231(0.4); 1.167(1.5); |
| | | | 1.159(1.4); 1.069(1.6); 0.870(9.7); 0.864(7.5); 0.850(5.5); 0.828(0.6); |
| | | | 0.146(3.5); 0.031(0.4); 0.008(27.0); 0.000(791.5); −0.009(29.5); −0.150(3.6) |
| I-193 | 3.95 | 4.12 | Example I-193: ¹H-NMR(400.0 MHz, d₆-DMSO): δ = |
| | | | 19.987(0.4); 10.940(6.0); 8.938(3.0); 8.926(2.8); 8.525(8.5); 8.311(1.8); 7.976 |
| | | | (8.5); 7.972(9.2); 7.836(9.0); 7.816(10.7); 7.795(8.3); 7.775(10.4); 7.643(9.9); |
| | | | 7.622(8.3); 7.556(10.1); 7.535(8.8); 7.439(0.4); 4.654(8.0); 4.638(8.2); 4.609(0.5); |
| | | | 4.339(0.5); 3.636(0.4); 3.560(0.4); 3.331(1283.7); 2.671(7.4); 2.618(0.7); |
| | | | 2.506(975.7); 2.502(1202.1); 2.498(1008.1); 2.359(1.7); 2.329(7.3); 2.278(1.2); |
| | | | 2.046(2.2); 2.035(3.0); 2.019(2.2); 1.389(10.7); 1.377(4.2); 1.236(0.9); 1.186(9.1); |
| | | | 1.045(3.0); 1.030(2.9); 0.867(16.0); 0.146(1.1); 0.007(55.0); 0.005(91.4); |
| | | | 0.000(244.8); −0.149(1.2) |
| I-194 | 3.74 | 3.81 | Example I-194: ¹H-NMR(400.0 MHz, d₆-DMSO): δ = |
| | | | 10.957(7.1); 8.952(1.7); 8.935(3.1); 8.921(1.6); 8.534(11.1); 8.531(11.4); |
| | | | 8.312(2.5); 7.982(12.2); 7.978(12.2); 7.901(1.4); 7.893(13.3); 7.888(4.4); |
| | | | 7.876(4.4); 7.871(15.2); 7.864(1.6); 7.838(13.3); 7.834(4.8); 7.822(4.9); 7.817(16.0); |
| | | | 7.557(12.7); 7.536(11.1); 7.455(10.3); 7.434(10.0); 7.419(4.3); 7.407(2.0); |
| | | | 7.314(1.6); 7.303(4.1); 7.291(2.0); 4.657(8.9); 4.641(9.0); 3.432(0.4); 3.324(509.3); |
| | | | 2.675(3.0); 2.671(4.2); 2.666(3.0); 2.524(9.7); 2.519(14.9); 2.511(246.6); |
| | | | 2.506(522.8); 2.502(702.3); 2.497(498.5); 2.493(232.6); 2.337(1.3); 2.333(2.9); |
| | | | 2.329(4.1); 2.324(3.0); 2.073(2.4); 2.052(1.9); 2.038(3.2); 2.021(2.0); 2.008(0.9); |
| | | | 1.045(0.5); 1.030(0.5); 0.870(15.9); 0.861(10.2); 0.850(8.0); 0.146(0.9); |
| | | | 0.008(7.0); 0.000(228.8); −0.009(7.0); −0.150(0.9) |
| I-195 | 4.08 | 4.22 | Example I-195: ¹H-NMR(400.0 MHz, d₆-DMSO): δ = |
| | | | 11.019(1.0); 8.569(1.4); 8.565(1.5); 8.345(0.5); 8.314(0.5); 8.024(1.0); 8.003(1.4); |
| | | | 7.988(1.6); 7.984(1.6); 7.879(1.4); 7.858(1.0); 3.317(30.1); 3.245(0.6); 3.229(1.1); |
| | | | 3.212(0.6); 2.671(0.4); 2.650(0.4); 2.524(0.7); 2.520(1.0); 2.511(17.4); |
| | | | 2.506(37.6); 2.502(50.9); 2.497(36.2); 2.493(16.8); 2.068(0.4); 2.052(0.8); |
| | | | 2.038(0.6); 2.018(0.5); 2.010(0.5); 1.790(0.4); 1.782(0.4); 1.756(0.5); 1.749(0.4); |
| | | | 1.506(0.5); 1.499(0.4); 1.475(0.5); 1.467(0.4); 1.398(16.0); 1.055(0.4); 1.049(0.4); |
| | | | 1.024(0.4); 1.020(0.4); 0.884(2.8); 0.869(1.8); 0.000(3.3) |
| I-196 | 4.30 | 4.37 | Example I-196: ¹H-NMR(400.0 MHz, d₆-DMSO): δ = |
| | | | 11.015(1.6); 9.029(0.4); 9.013(0.7); 8.999(0.3); 8.586(2.6); 8.582(2.8); 8.313(7.2); |
| | | | 8.247(0.4); 8.241(3.5); 8.236(1.2); 8.224(1.1); 8.219(3.8); 8.213(0.5); 8.037(4.7); |
| | | | 8.033(3.7); 8.018(2.6); 7.885(2.6); 7.864(2.1); 7.635(2.9); 7.613(2.8); |
| | | | 4.712(2.1); 4.696(2.1); 3.328(62.8); 3.323(59.3); 3.296(2.0); 2.676(0.5); 2.671 |
| | | | (0.8); 2.667(0.5); 2.524(1.8); 2.520(2.7); 2.511(44.2); 2.507(95.2); 2.502(129.2); |
| | | | 2.497(92.4); 2.493(43.4); 2.333(0.5); 2.329(0.8); 2.324(0.6); 2.065(0.5); 2.052(0.7); |
| | | | 2.035(0.5); 1.398(16.0); 0.877(4.5); 0.866(2.4); 0.857(2.1); 0.000(8.1) |
| I-197 | 3.66 | 3.76 | Example I-197: ¹H-NMR(400.0 MHz, d₆-DMSO): δ = |
| | | | 11.016(2.2); 1.004(1.3); 8.535(1.7); 8.531(1.8); 8.521(2.9); 8.517(3.0); 8.313 |
| | | | (1.8); 8.275(0.7); 8.253(0.8); 8.141(1.3); 8.121(1.3); 7.939(2.0); 7.933(3.6); |
| | | | 7.929(3.3); 7.907(2.3); 7.901(4.0); 7.885(3.2); 7.879(4.4); 7.539(3.4); 7.518(3.1); |
| | | | 3.904(0.3); 3.881(0.5); 3.852(0.6); 3.846(0.6); 3.834(0.5); 3.826(0.5); 3.318(556.1); |
| | | | 3.142(0.6); 3.132(0.4); 2.985(0.4); 2.767(0.4); 2.746(0.5); 2.738(0.9); 2.728(0.5); |
| | | | 2.708(0.5); 2.675(2.4); 2.670(3.4); 2.666(2.6); 2.661(1.3); 2.524(8.0); |
| | | | 2.510(198.4); 2.506(418.9); 2.501(565.7); 2.497(409.9); 2.492(198.1); 2.332(2.4); |
| | | | 2.328(3.3); 2.324(2.5); 2.087(1.0); 2.059(1.3); 2.049(1.5); 2.034(1.1); 2.030(1.2); |
| | | | 2.015(0.9); 2.000(0.3); 1.962(1.2); 1.938(1.9); 1.928(1.8); 1.778(0.4); |
| | | | 1.749(0.6); 1.740(0.6); 1.722(0.6); 1.698(1.0); 1.690(1.0); 1.665(1.1); 1.660(1.0); |
| | | | 1.635(0.5); 1.625(0.5); 1.612(0.3); 1.587(0.6); 1.559(0.5); 1.423(0.5); 1.398(16.0); |
| | | | 1.371(0.9); 1.339(0.4); 1.299(0.5); 0.882(3.9); 0.873(6.9); 0.859(4.1); |
| | | | 0.146(1.1); 0.008(8.1); 0.000(246.0); −0.008(9.8); −0.150(1.1) |
| I-198 | 3.75 | 3.85 | Example I-198: ¹H-NMR(400.0 MHz, d₆-DMSO): δ = |
| | | | 10.976(5.5); 8.516(8.2); 8.512(8.3); 8.347(1.4); 8.331(3.0); 8.313(3.3); 7.940(9.2); |
| | | | 7.936(9.0); 7.911(1.0); 7.904(9.6); 7.898(3.1); 7.887(3.2); 7.881(11.1); 7.874(1.1); |
| | | | 7.627(0.4); 7.614(0.3); 7.597(0.4); 7.565(0.3); 7.540(6.3); 7.520(5.5); |
| | | | 3.318(483.7); 3.277(0.3); 3.239(3.4); 3.223(6.2); 3.206(3.4); 2.675(2.9); 2.670(3.9); |
| | | | 2.666(2.8); 2.661(1.5); 2.648(2.1); 2.640(1.2); 2.627(0.8); 2.618(1.1); 2.610(0.6); |
| | | | 2.524(7.6); 2.519(11.8); 2.511(195.4); 2.506(418.1); 2.502(563.1); |
| | | | 2.497(400.1); 2.492(186.7); 2.337(1.1); 2.333(2.3); 2.328(3.3); 2.324(2.4); |
| | | | 2.075(0.6); 2.059(2.3); 2.045(4.3); 2.029(3.2); 2.014(3.2); 1.786(2.2); 1.779(2.4); |
| | | | 1.753(2.6); 1.745(2.6); 1.616(0.4); 1.606(0.7); 1.598(0.8); 1.589(1.0); 1.579(1.2); |
| | | | 1.570(1.0); 1.560(0.8); 1.551(0.8); 1.537(1.1); 1.529(1.1); 1.505(2.6); 1.496 |
| | | | (2.5); 1.473(2.5); 1.466(2.5); 1.442(1.0); 1.434(0.9); 1.398(3.5); 1.305(0.4); |
| | | | 1.233(0.3); 1.085(0.9); 1.077(1.1); 1.052(2.4); 1.045(2.4); 1.015(2.2); 0.991(0.8); |
| | | | 0.982(0.7); 0.878(16.0); 0.864(9.5); 0.000(6.8) |
| I-199 | | 3.21 | Example I-199: ¹H-NMR(400.0 MHz, d₆-DMSO): δ = |
| | | | 11.045(4.2); 8.460(5.7); 8.456(6.0); 8.313(2.0); 7.894(6.6); 7.878(2.2); 7.872(7.7); |
| | | | 7.865(0.9); 7.623(0.5); 7.615(0.4); 7.597(0.3); 7.555(6.3); 7.552(6.5); 7.535(4.8); |
| | | | 7.515(4.0); 4.001(0.9); 3.970(0.9); 3.505(0.8); 3.472(1.2); 3.402(0.9); |
| | | | 3.376(1.4); 3.368(1.1); 3.316(664.7); 3.271(1.0); 3.262(1.0); 3.253(0.8); 3.237 |
| | | | (0.7); 3.220(0.5); 3.207(0.7); 3.197(1.0); 3.186(1.5); 3.174(1.1); 3.163(0.8); |
| | | | 3.153(0.4); 2.679(1.9); 2.675(4.1); 2.670(5.9); 2.666(4.4); 2.661(2.2); |
| | | | 2.523(13.2); 2.519(20.2); 2.510(333.7); 2.506(719.1); 2.501(979.8); 2.496(710.6); |
| | | | 2.492(340.3); 2.337(1.9); 2.332(4.1); 2.328(5.8); 2.323(4.4); 2.064(0.4); 2.046(1.1); |
| | | | 2.033(1.8); 2.016(1.6); 2.003(1.1); 1.981(0.9); 1.891(0.8); 1.877(0.9); 1.790(1.1); |
| | | | 1.781(1.3); 1.767(1.4); 1.758(1.3); 1.743(1.1); 1.398(16.0); 0.860(9.4); 0.849(5.2); |
| | | | 0.840(4.5); 0.818(0.3); 0.146(1.1); 0.008(7.8); |
| | | | 0.000(274.5); −0.009(9.7); −0.034(0.4); −0.150(1.3) |
| I-200 | 3.51 | 3.59 | Example I-200: ¹H-NMR(400.0 MHz, d₆-DMSO): δ = |
| | | | 10.984(5.3); 8.515(7.2); 8.511(7.4); 8.336(2.0); 8.322(1.0); 7.947(8.9); 7.943(8.9); |
| | | | 7.912(1.0); 7.905(8.5); 7.900(3.0); 7.889(3.0); 7.883(9.8); 7.876(1.2); 7.542(6.2); |
| | | | 7.522(5.6); 3.388(2.3); 3.371(6.1); 3.338(166.4); 3.330(149.6); 2.962(2.0); |
| | | | 2.677(1.0); 2.672(1.3); 2.667(1.0); 2.663(0.5); 2.526(7.9); 2.521(6.0); 2.512(79.9); |
| | | | 2.508(172.4); 2.503(221.7); 2.498(159.1); 2.494(77.1); 2.334(0.9); 2.330(1.3); |
| | | | 2.325(1.0); 2.321(0.5); 2.074(0.5); 2.059(1.5); 2.043(1.9); 2.028(1.6); |
| | | | 2.013(0.5); 1.646(1.0); 1.628(3.0); 1.608(5.1); 1.589(5.9); 1.567(3.2); 1.549(1.2); |
| | | | 1.460(1.2); 1.448(1.8); 1.440(3.0); 1.431(1.8); 1.423(3.5); 1.410(1.5); 1.401(1.8); |
| | | | 1.383(0.5); 1.046(0.4); 1.031(0.4); 0.878(16.0); 0.864(8.5); 0.861(8.1); |
| | | | 0.008(1.2); 0.000(37.6); −0.008(1.5) |
| I-201 | 3.21 | 3.25 | Example I-201: ¹H-NMR(400.0 MHz, d₆-DMSO): δ = |
| | | | 10.797(5.3); 8.513(7.5); 8.509(7.8); 8.146(1.5); 7.881(16.0); 7.865(3.3); 7.860 |
| | | | (10.6); 7.853(1.3); 7.534(6.6); 7.514(6.0); 5.006(0.9); 4.990(1.2); 4.980(3.4); |
| | | | 4.964(6.2); 4.942(3.6); 4.916(1.0); 4.424(1.8); 4.400(4.0); 4.376(2.6); 4.267(2.4); |
| | | | 4.252(2.6); 4.242(1.9); 4.227(2.0); 3.941(0.7); 3.926(1.3); 3.919(1.4); 3.911(1.0); |
| | | | 3.904(2.2); 3.897(1.0); 3.888(1.2); 3.882(1.3); 3.866(0.6); 3.344(92.8); 2.677(0.6); |
| | | | 2.673(0.8); 2.668(0.6); 2.526(1.8); 2.512(48.3); 2.508(100.6); 2.504(134.7); |
| | | | 2.499(97.5); 2.495(47.7); 2.335(0.6); 2.330(0.8); 2.326(0.6); 2.074(4.7); |
| | | | 2.053(1.7); 2.038(2.4); 2.022(1.7); 2.007(0.7); 0.902(7.4); 0.895(7.8); 0.883(15.1); |
| | | | 0.008(0.7); 0.000(22.6); −0.008(0.9) |
| I-202 | 3.43 | 3.46 | Example I-202: ¹H-NMR(400.0 MHz, d₆-DMSO): δ = |
| | | | 11.037(6.0); 10.927(1.6); 8.516(8.8); 8.512(11.1); 8.314(0.3); 7.909(10.4); |
| | | | 7.904(3.6); 7.891(7.9); 7.887(13.7); 7.880(1.5); 7.748(8.8); 7.744(9.0); 7.537(6.8); |
| | | | 7.517(6.1); 6.022(0.7); 6.015(0.8); 6.004(0.7); 5.996(0.7); 5.755(16.0); 5.002 |
| | | | (2.8); 4.991(3.4); 4.982(3.2); 4.972(2.9); 3.967(0.9); 3.952(2.0); 3.940(1.6); |
| | | | 3.925(2.4); 3.909(1.2); 3.784(0.3); 3.775(0.6); 3.766(0.5); 3.754(0.4); 3.740(1.2); |
| | | | 3.730(0.4); 3.721(2.6); 3.713(1.2); 3.703(1.5); 3.694(2.2); 3.676(1.0); 3.620(0.3); |
| | | | 3.599(0.5); 3.580(0.4); 3.569(0.5); 3.321(186.4); 2.676(0.7); 2.672(0.9); |
| | | | 2.667(0.7); 2.542(0.3); 2.525(2.2); 2.520(3.5); 2.512(55.9); 2.507(119.5); 2.503 |
| | | | (161.6); 2.498(116.3); 2.494(55.4); 2.390(0.4); 2.381(0.4); 2.363(0.7); 2.342(1.0); |
| | | | 2.334(1.2); 2.329(1.3); 2.321(1.6); 2.309(1.0); 2.302(1.5); 2.289(2.6); 2.282(0.8); |
| | | | 2.270(2.7); 2.251(1.6); 2.237(2.0); 2.226(2.1); 2.212(1.2); 2.205(1.0); 2.194(0.9); |
| | | | 2.180(0.5); 2.063(1.6); 2.045(4.2); 2.037(3.8); 2.029(5.2); 2.011(3.4); |
| | | | 1.995(1.7); 1.980(1.0); 1.964(0.5); 0.912(3.5); 0.897(4.5); 0.876(15.1); 0.863 |
| | | | (8.1); 0.860(7.9); 0.008(0.7); 0.000(21.1); −0.009(0.8) |
| I-203 | 3.32 | | Example I-203: ¹H-NMR(400.0 MHz, d₆-DMSO): δ = |
| | | | 19.971(0.8); 9.525(3.0); 9.399(0.9); 9.383(1.5); 8.553(3.7); 8.549(3.8); 8.313(5.6); |
| | | | 8.039(3.3); 8.035(3.5); 7.936(3.9); 7.914(4.3); 7.831(3.9); 7.810(4.6); 7.792(0.8); |
| | | | 7.547(5.4); 7.528(5.2); 7.493(0.8); 4.661(2.9); 4.646(2.7); 3.791(0.9); |
| | | | 3.318(1401.4); 2.675(11.5); 2.670(15.8); 2.666(12.1); 2.524(38.8); 2.510(1004.1); |
| | | | 2.506(2065.3); 2.501(2730.6); 2.497(1951.7); 2.492(928.4); 2.333(11.6); |
| | | | 2.328(16.0); 2.324(11.6); 1.590(1.1); 1.398(7.2); 1.236(0.8); 1.057(1.3); 1.048(3.2); |
| | | | 1.042(3.1); 1.032(1.3); 0.689(2.0); 0.681(2.3); 0.671(4.9); 0.273(2.3); 0.259(2.2); |
| | | | 0.146(15.3); 0.029(1.6); 0.008(120.8); 0.000(3454.7); −0.008(124.4); −0.149(15.3) |
| I-204 | 3.27 | | Example I-204: ¹H-NMR(400.0 MHz, d₆-DMSO): δ = |
| | | | 10.645(1.9); 8.951(1.1); 8.936(1.0); 8.513(2.0); 8.311(8.7); 7.982(4.8); 7.978(4.8); |
| | | | 7.917(5.1); 7.896(5.9); 7.834(5.0); 7.813(6.1); 7.559(3.8); 7.549(5.6); 7.539(3.7); |
| | | | 7.528(4.6); 4.648(3.9); 4.633(3.7); 3.433(0.4); 3.345(262.9); 3.342(199.2); |
| | | | 3.339(169.9); 3.335(166.6); 3.330(188.6); 2.672(1.5); 2.667(1.2); 2.525(4.4); |
| | | | 2.511(92.9); 2.507(195.7); 2.502(264.4); 2.498(190.8); 2.493(91.3); 2.473(0.5); |
| | | | 2.334(1.1); 2.329(1.5); 2.325(1.2); 2.155(16.0); 1.988(0.4); 1.398(1.3); |
| | | | 1.233(0.3); 0.008(2.3); 0.000(63.7); −0.008(2.6); −0.150(0.3) |
| I-205 | 4.62 | | Example I-205: ¹H-NMR(400.0 MHz, d₆-DMSO): δ = |
| | | | 9.923(4.0); 9.494(1.1); 9.478(2.4); 9.462(1.1); 8.439(5.5); 8.435(5.7); 8.084(5.9); |
| | | | 8.080(5.8); 7.945(6.3); 7.923(7.2); 7.830(6.3); 7.810(7.4); 7.550(8.4); 7.530 |
| | | | (7.2); 4.650(4.6); 4.634(4.5); 3.323(84.3); 2.676(0.6); 2.672(0.9); 2.667(0.7); |
| | | | 2.525(2.0); 2.511(53.7); 2.507(110.5); 2.502(146.8); 2.498(106.4); 2.494(51.7); |
| | | | 2.334(0.6); 2.329(0.8); 2.325(0.6); 1.989(0.7); 1.716(2.0); 1.702(5.0); 1.694(5.4); |
| | | | 1.682(2.5); 1.553(0.3); 1.511(2.7); 1.499(5.3); 1.491(5.3); 1.477(1.9); 1.398(16.0); |
| | | | 1.175(0.4); 0.008(2.2); 0.000(62.5); −0.008(2.4) |
| I-206 | 4.45 | | Example I-206: ¹H-NMR(400.0 MHz, d₆-DMSO): δ = |
| | | | 9.698(0.7); 9.160(0.4); 8.531(1.1); 8.527(1.2); 8.312(3.0); 8.011(1.2); 8.007(1.2); |
| | | | 7.942(1.2); 7.920(1.5); 7.835(1.3); 7.814(1.4); 7.549(1.8); 7.529(1.6); 4.667 |
| | | | (0.9); 4.652(0.9); 3.428(0.4); 3.419(0.4); 3.335(230.2); 3.329(176.2); 3.325(176.6); |
| | | | 2.675(1.3); 2.671(1.5); 2.666(1.2); 2.524(4.1); 2.511(100.6); 2.506(211.0); |
| | | | 2.502(283.2); 2.497(203.3); 2.493(97.0); 2.445(0.3); 2.333(1.3); 2.328(1.6); |
| | | | 2.324(1.2); 1.283(16.0); 0.008(2.0); 0.000(59.6); −0.008(2.2); −0.150(0.4) |
| I-207 | 4.51 | | Example I-207: ¹H-NMR(400.0 MHz, d₆-DMSO): δ = |
| | | | 10.576(2.4); 8.958(0.7); 8.943(1.1); 8.928(0.5); 8.580(3.9); 8.577(4.1); 8.314(0.4); |
| | | | 7.984(4.8); 7.980(5.0); 7.932(4.5); 7.910(5.3); 7.836(4.6); 7.815(5.7); 7.551(8.2); |
| | | | 7.531(7.3); 4.652(3.5); 4.637(3.5); 4.039(0.4); 4.021(0.4); 3.334(50.9); |
| | | | 3.327(39.9); 2.998(0.8); 2.979(1.3); 2.959(0.9); 2.677(0.3); 2.672(0.5); 2.667 |
| | | | (0.3); 2.525(1.1); 2.521(1.7); 2.512(27.2); 2.508(58.8); 2.503(80.1); 2.498(57.8); |
| | | | 2.494(27.5); 2.334(0.3); 2.330(0.5); 2.325(0.3); 1.989(1.8); 1.915(0.4); |
| | | | 1.895(0.8); 1.885(1.3); 1.870(1.2); 1.864(1.5); 1.858(1.1); 1.849(0.9); 1.834(0.5); |
| | | | 1.792(0.4); 1.775(1.2); 1.756(1.6); 1.743(1.1); 1.738(1.1); 1.725(0.9); 1.711(0.9); |
| | | | 1.700(0.9); 1.694(1.1); 1.685(1.3); 1.676(1.4); 1.673(1.4); 1.659(1.1); 1.639(0.4); |
| | | | 1.634(0.4); 1.602(0.5); 1.579(1.4); 1.569(1.4); 1.562(1.7); 1.549(1.2); 1.543(0.9); |
| | | | 1.397(16.0); 1.193(0.5); 1.176(0.9); 1.158(0.5); 0.008(1.0); 0.000(31.4); −0.008(1.2) |
| I-208 | 3.71 | 3.81 | Example I-208: ¹H-NMR(400.0 MHz, d₆-DMSO): δ = |
| | | | 10.979(5.0); 8.964(1.4); 8.948(3.0); 8.933(1.4); 8.529(6.2); 8.525(6.5); 8.314(1.5); |
| | | | 7.990(6.8); 7.986(7.0); 7.839(7.8); 7.823(5.4); 7.819(10.7); 7.803(3.4); 7.731(4.0); |
| | | | 7.713(3.0); 7.693(5.2); 7.673(2.8); 7.561(7.7); 7.540(8.6); 7.516(1.9); |
| | | | 5.754(16.0); 4.661(5.7); 4.646(5.7); 3.571(0.4); 3.328(68.8); 3.304(0.6); 2.527(0.8); |
| | | | 2.514(17.4); 2.510(35.3); 2.505(47.1); 2.501(34.9); 2.496(17.6); 2.075(0.5); |
| | | | 2.062(1.1); 2.057(1.2); 2.051(1.0); 2.044(2.0); 2.031(1.2); 2.027(1.2); 2.013(0.5); |
| | | | 1.397(0.5); 0.878(7.2); 0.873(8.6); 0.869(7.3); 0.853(5.0); 0.831(0.4); |
| | | | 0.008(0.4); 0.000(11.5); −0.009(0.5) |
| I-209 | 4.33 | 4.34 | Example I-209: ¹H-NMR(400.0 MHz, d₆-DMSO): δ = |
| | | | 11.051(6.5); 9.049(1.7); 9.034(3.7); 9.018(1.8); 8.567(7.9); 8.563(8.2); 8.241(10.1); |
| | | | 8.219(11.1); 8.055(8.4); 8.051(8.6); 8.001(2.1); 7.974(1.9); 7.929(1.4); 7.909(2.8); |
| | | | 7.892(2.5); 7.859(4.3); 7.836(2.3); 7.634(9.1); 7.613(8.7); 5.756(6.0); |
| | | | 4.711(6.9); 4.695(6.9); 3.569(0.4); 3.324(119.7); 2.677(0.5); 2.672(0.8); 2.668(0.6); |
| | | | 2.525(1.9); 2.512(42.9); 2.508(89.9); 2.503(125.7); 2.499(95.2); 2.494(46.9); |
| | | | 2.334(0.5); 2.330(0.7); 2.326(0.6); 2.085(0.6); 2.068(1.7); 2.054(2.4); 2.038(1.9); |
| | | | 2.023(0.7); 1.232(0.4); 0.879(16.0); 0.865(8.9); 0.862(8.5); 0.008(1.7); |
| | | | 0.000(50.7); −0.008(1.9) |
| I-210 | 3.88 | 4.04 | Example I-210: ¹H-NMR(400.0 MHz, d₆-DMSO): δ = |
| | | | 11.374(0.7); 10.208(7.0); 9.303(1.7); 9.287(3.7); 9.272(1.8); 9.000(0.5); 8.984 |
| | | | (0.3); 8.490(0.4); 8.374(9.4); 8.370(10.0); 8.314(0.7); 8.090(1.2); 8.083(10.2); |
| | | | 8.080(10.3); 8.072(2.0); 8.069(2.2); 7.993(0.4); 7.973(0.4); 7.950(1.5); 7.943(11.3); |
| | | | 7.938(4.4); 7.926(4.3); 7.920(13.6); 7.913(2.0); 7.897(1.0); 7.885(0.5); 7.832(11.2); |
| | | | 7.811(14.4); 7.801(2.0); 7.792(1.6); 7.663(0.5); 7.645(0.7); 7.625(0.6); |
| | | | 7.551(16.0); 7.531(14.1); 7.515(1.9); 7.498(1.3); 7.483(1.0); 7.480(1.0); 7.460(1.2); |
| | | | 7.456(1.0); 7.432(0.4); 6.927(0.6); 6.923(0.6); 6.261(0.5); 4.752(0.6); |
| | | | 4.737(0.5); 4.718(0.3); 4.656(7.9); 4.640(8.0); 4.588(0.7); 4.573(0.7); 3.321(178.1); |
| | | | 2.892(2.5); 2.732(2.2); 2.691(0.4); 2.676(1.1); 2.672(1.5); 2.667(1.1); 2.620(0.4); |
| | | | 2.600(0.5); 2.584(0.5); 2.570(0.6); 2.542(1.6); 2.525(4.3); 2.512(83.8); |
| | | | 2.507(175.3); 2.503(244.0); 2.498(185.1); 2.494(91.1); 2.334(1.0); 2.330(1.4); |
| | | | 2.325(1.0); 2.074(12.0); 1.798(1.0); 1.779(4.4); 1.770(12.7); 1.766(8.0); 1.757(7.7); |
| | | | 1.753(12.4); 1.744(4.5); 1.725(0.9); 1.337(1.0); 1.262(0.4); 1.233(0.5); |
| | | | 1.226(1.4); 0.941(0.5); 0.924(0.5); 0.008(0.8); 0.000(24.0); −0.009(0.8) |
| I-211 | 4.01 | 4.16 | Example I-211: ¹H-NMR(400.0 MHz, d₆-DMSO): δ = |
| | | | 11.024(6.8); 8.965(1.9); 8.950(4.1); 8.934(2.0); 8.517(4.8); 8.376(0.4); 8.366(0.5); |
| | | | 8.353(0.4); 8.344(0.6); 8.314(0.8); 8.297(0.4); 8.273(0.3); 8.227(11.4); 8.205(12.5); |
| | | | 8.182(0.4); 8.161(0.4); 8.133(0.4); 8.112(0.4); 8.089(0.4); 8.046(0.4); |
| | | | 8.013(9.8); 8.009(10.0); 7.983(0.8); 7.969(0.9); 7.960(0.7); 7.940(0.4); 7.916(10.5); |
| | | | 7.899(4.0); 7.894(12.0); 7.887(1.6); 7.840(10.3); 7.820(12.3); 7.694(0.4); |
| | | | 7.649(10.6); 7.627(10.0); 7.601(0.3); 7.588(0.3); 7.557(10.6); 7.537(16.0); |
| | | | 7.518(7.1); 4.658(7.5); 4.643(7.5); 3.994(13.1); 3.370(23.9); 2.676(1.0); 2.672 |
| | | | (1.4); 2.667(1.0); 2.525(3.3); 2.511(79.6); 2.507(166.9); 2.503(232.7); 2.498(178.0); |
| | | | 2.334(1.0); 2.330(1.4); 2.325(1.0); 0.000(4.4) |
| I-212 | 3.34 | 3.44 | Example I-212: ¹H-NMR(400.0 MHz, d₆-DMSO): δ = |
| | | | 10.958(7.7); 9.983(0.5); 9.167(1.8); 9.152(3.9); 9.136(1.9); 8.716(16.0); 8.551 |
| | | | (8.6); 8.547(8.9); 8.503(0.7); 8.422(0.5); 8.412(0.5); 8.409(0.3); 8.313(0.5); |
| | | | 8.215(16.0); 8.088(8.9); 8.084(8.9); 8.067(0.5); 8.045(0.6); 7.977(10.0); 7.972(3.6); |
| | | | 7.960(3.7); 7.955(11.4); 7.949(1.4); 7.839(10.0); 7.818(12.0); 7.789(0.8); |
| | | | 7.578(7.4); 7.563(12.5); 7.543(9.5): 7.516(0.4); 4.667(7.0); 4.651(7.1); 4.619(0.5); |
| | | | 4.604(0.5); 3.320(119.6); 2.676(0.8): 2.672(1.1); 2.667(0.8); 2.542(0.8); 2.525(2.4); |
| | | | 2.511(65.4); 2.507(136.0); 2.502(188.9); 2.498(141.3); 2.494(69.2); 2.334(0.8); |
| | | | 2.329(1.2); 2.325(0.9); 2.074(4.5); 0.000(1.9) |
| I-213 | 4.02 | 4.14 | Example I-213: ¹H-NMR(400.0 MHz, d₆-DMSO): δ = |
| | | | 10.852(8.2); 8.960(2.2); 8.944(4.7): 8.929(2.3); 8.574(4.8); 8.017(11.7); 8.013 |
| | | | (11.3); 7.944(12.3); 7.940(4.4); 7.922(14.1); 7.915(1.7); 7.834(12.3); 7.813(14.6); |
| | | | 7.563(9.2); 7.547(16.0): 7.527(11.0): 4.647(9.1); 4.631(9.0); 3.324(228.5); |
| | | | 3.282(1.3); 2.916(0.4); 2.884(1.7): 2.863(3.1); 2.858(3.1); 2.839(4.7); 2.822(4.8); |
| | | | 2.809(5.3); 2.789(3.3); 2.676(0.8); 2.672(1.1); 2.668(0.9); 2.525(3.2); 2.511 |
| | | | (70.7); 2.507(139.0); 2.503(185.9); 2.498(139.7); 2.494(69.0); 2.334(0.8); 2.330(1.1); |
| | | | 2.325(0.8); 2.075(2.7); 0.146(0.9); 0.008(7.3); 0.000(197.4); −0.008(8.2); −0.150(0.9) |
| I-214 | 3.35 | 3.45 | Example I-214: ¹H-NMR(400.0 MHz, d₆-DMSO): δ = |
| | | | 10.984(7.4); 9.844(2.8); 9.472(2.7); 8.801(2.0); 8.786(4.6); 8.771(2.4); 8.533(9.6); |
| | | | 8.529(9.4); 8.313(5.4); 7.986(10.2): 7.982(10.2); 7.917(11.1); 7.895(12.8); |
| | | | 7.879(10.6); 7.858(11.9); 7.545(8.0); 7.525(7.5); 7.400(10.3); 7.379(9.8); |
| | | | 4.604(7.9); 4.588(7.3); 3.490(0.8); 3.434(0.8); 3.422(1.1); 3.417(1.0); 3.316(1833.9); |
| | | | 2.976(0.8); 2.823(0.8); 2.793(0.8); 2.709(0.9); 2.675(11.9); 2.670(16.0); |
| | | | 2.666(11.6); 2.603(1.8); 2.524(43.9); 2.510(935.5); 2.506(1901.1); 2.501(2594.7); |
| | | | 2.497(1940.3); 2.493(947.0); 2.332(11.0); 2.328(15.5); 2.324(10.8); 2.086(8.1); |
| | | | 2.049(0.9); 2.037(1.9); 2.019(3.0): 2.005(2.0); 1.398(3.9); 1.352(0.8); 1.257(0.8); |
| | | | 1.235(2.1); 0.894(0.8); 0.862(14.5); 0.853(9.7); 0.842(7.3); 0.146(7.2); |
| | | | 0.045(0.8); 0.008(56.2); 0.000(1619.3); −0.008(57.0); −0.150(7.4) |
| I-215 | 3.84 | 3.85 | Example I-215: ¹H-NMR(400.0 MHz, d₆-DMSO): δ = |
| | | | 9.424(2.3); 9.408(5.1); 9.392(2.4); 8.315(0.3); 8.183(11.4); 8.179(12.2); 8.055 |
| | | | (11.8); 8.051(11.2); 8.026(1.5); 8.018(13.2); 8.013(4.5); 8.001(4.8); 7.996(14.9); |
| | | | 7.989(1.7); 7.816(13.2); 7.795(16.0); 7.554(10.3); 7.548(15.3); 7.532(11.0); |
| | | | 7.527(12.7); 5.755(0.9); 4.629(9.0); 4.613(9.0); 3.487(39.9); 3.320(112.3); |
| | | | 3.178(0.5); 3.164(0.5); 2.677(0.6); 2.672(0.9); 2.668(0.6); 2.526(2.2); 2.512(49.6); |
| | | | 2.508(103.7); 2.503(143.8); 2.498(106.8); 2.494(50.7); 2.334(0.6); 2.330(0.8); |
| | | | 2.325(0.6); 1.843(0.6); 1.831(1.4); 1.823(1.7); 1.812(2.5); 1.802(1.8); 1.793(1.4); |
| | | | 1.781(0.7); 1.233(0.4); 0.939(2.0); 0.929(5.6); 0.921(7.9); 0.910(6.2); 0.903(2.5); |
| | | | 0.796(2.4); 0.788(5.6); 0.780(5.0); 0.769(5.9); 0.760(4.5); 0.751(1.7); |
| | | | 0.146(0.4); 0.008(3.3); 0.000(101.4); −0.009(3.2); −0.150(0.4) |
| I-216 | 3.74 | 3.85 | Example I-216: ¹H-NMR(400.0 MHz, d₆-DMSO): δ = |
| | | | 10.967(6.4); 8.636(4.0); 8.537(7.8); 7.953(8.2); 7.949(7.8); 7.919(8.2); 7.897(9.2); |
| | | | 7.546(7.2); 7.525(6.6); 5.754(5.3); 3.584(1.5); 3.570(1.8); 3.561(1.6); 3.320(67.6); |
| | | | 2.672(0.6); 2.503(96.0); 2.330(0.5); 2.084(1.0); 2.068(2.3); 2.055(3.3); |
| | | | 2.040(3.1); 2.025(1.9); 2.008(1.2); 1.984(0.6); 1.832(0.6); 1.819(1.0); 1.806 |
| | | | (1.1); 1.796(1.5); 1.782(1.6); 1.768(1.0); 1.761(1.0); 1.746(0.5); 1.398(0.8); |
| | | | 1.234(0.9); 0.876(16.0); 0.864(9.6); 0.860(9.4); 0.000(0.8) |
| I-217 | 3.89 | 4.02 | Example I-217: ¹H-NMR(400.0 MHz, d₆-DMSO): δ = |
| | | | 9.432(1.0); 9.416(2.1); 9.400(1.0); 8.218(4.3); 8.071(4.5); 8.041(4.6); 8.019(5.1); |
| | | | 7.810(5.0); 7.790(5.8); 7.559(4.1); 7.534(7.4); 7.513(4.6); 4.614(3.9); 4.599 |
| | | | (4.0); 3.403(13.4); 3.320(25.0); 3.319(25.2); 2.799(0.7); 2.782(0.9); 2.766(0.7); |
| | | | 2.749(0.3); 2.673(0.4); 2.503(62.5); 2.330(0.4); 1.394(0.4); 1.095(1.0); 1.086 |
| | | | (0.4); 1.077(1.1); 1.068(0.9); 1.047(16.0); 1.031(15.7); 0.000(28.3) |
| I-218 | 4.02 | 4.17 | Example I-218: ¹H-NMR(400.0 MHz, d₆-DMSO): δ = |
| | | | 11.014(6.3); 8.746(3.5); 8.726(3.6); 8.537(9.0); 8.533(9.2); 8.313(3.2); 7.921(10.3); |
| | | | 7.917(10.3); 7.908(1.3); 7.901(10.7); 7.895(3.6); 7.884(3.7); 7.878(12.3); |
| | | | 7.871(1.4); 7.843(10.1); 7.839(3.6); 7.827(4.0); 7.822(12.7); 7.636(11.1); |
| | | | 7.616(9.1); 7.535(6.8); 7.515(6.2); 5.440(0.3); 5.271(0.4); 5.253(1.9); 5.235(2.8); |
| | | | 5.216(2.0); 5.198(0.5); 4.178(0.4); 4.154(0.4); 4.039(0.8); 3.318(272.8); 3.295 |
| | | | (1.3); 2.680(0.7); 2.675(1.5); 2.671(2.1); 2.666(1.6); 2.662(0.8); 2.541(1.3); |
| | | | 2.524(5.0); 2.520(7.7); 2.511(122.8); 2.506(260.1); 2.502(349.0); 2.497(251.2); |
| | | | 2.493(120.9); 2.338(0.7); 2.333(1.5); 2.329(2.2); 2.324(1.6); 2.190(1.2); 2.165 |
| | | | (0.3); 2.086(0.6); 2.071(1.8); 2.056(2.4); 2.040(1.9); 2.025(0.7); 1.564(13.4); |
| | | | 1.546(13.4); 1.235(0.4); 0.882(16.0); 0.869(8.2); 0.863(8.6); 0.146(0.7); |
| | | | 0.008(5.6); 0.000(191.1); −0.009(6.7); −0.150(0.7) |
| I-219 | 4.02 | 4.17 | Example I-219: ¹H-NMR(400.0 MHz, d₆-DMSO): δ = |
| | | | 11.015(5.9); 8.748(3.9); 8.728(3.7); 8.537(7.6); 8.533(6.4); 8.313(0.4); 7.922(8.8); |
| | | | 7.918(7.9); 7.914(4.4); 7.901(8.2); 7.896(5.6); 7.879(8.3); 7.843(8.0); 7.839(5.5); |
| | | | 7.823(8.6); 7.637(8.8); 7.617(7.0); 7.537(7.2); 7.516(6.3); 5.254(2.1); |
| | | | 5.237(2.9); 5.218(2.0); 3.328(285.6); 3.323(293.6); 3.184(0.4); 2.676(1.7); 2.671(1.7); |
| | | | 2.507(228.0); 2.503(235.0); 2.498(175.8); 2.334(1.6); 2.330(1.6); 2.072(2.2); |
| | | | 2.057(2.7); 2.043(2.2); 1.565(11.7); 1.547(11.0); 1.234(0.3); 0.882(16.0); |
| | | | 0.870(11.7); 0.048(0.5); 0.004(34.1); 0.000(53.7); −0.005(18.3) |
| I-220 | 3.35 | 3.39 | Example I-220: ¹H-NMR(400.0 MHz, d₆-DMSO): δ = |
| | | | 9.457(1.2); 9.441(2.5); 9.426(1.3); 8.182(5.6); 8.179(5.2); 8.087(4.8); 8.035(5.7); |
| | | | 8.013(6.2); 7.819(6.3); 7.799(7.3); 7.561(5.0); 7.543(10.7); 7.523(5.9); 4.624(4.8); |
| | | | 4.609(5.0); 3.435(16.8); 3.325(47.6); 3.312(2.1); 2.678(0.5); 2.674(0.4); |
| | | | 2.513(71.7); 2.509(88.5); 2.505(66.2); 2.336(0.6); 2.332(0.4); 2.178(16.0); |
| | | | 1.240(0.5) |
| I-221 | 4.20 | 4.20 | Example I-221: ¹H-NMR(400.0 MHz, d₆-DMSO): δ = |
| | | | 9.382(0.9); 9.366(1.9); 9.350(0.9); 8.314(0.3); 8.255(4.2); 8.251(4.4); 8.065(0.6); |
| | | | 8.058(5.0); 8.053(1.7); 8.041(1.8); 8.036(5.7); 8.028(0.6); 7.989(4.7); 7.985 |
| | | | (4.7); 7.811(5.3); 7.794(1.9); 7.790(6.4); 7.557(3.6); 7.532(6.6); 7.511(4.5); |
| | | | 4.617(3.4); 4.601(3.4); 3.949(0.9); 3.931(2.6); 3.913(2.7); 3.896(0.9); 3.319(72.0); |
| | | | 2.676(0.4); 2.672(0.5); 2.668(0.4); 2.632(0.6); 2.615(0.8); 2.598(0.6); 2.525 |
| | | | (1.1); 2.521(1.5); 2.512(26.2); 2.507(57.1); 2.503(81.1); 2.498(59.7); 2.494(27.9); |
| | | | 2.334(0.3); 2.330(0.5); 2.325(0.4); 1.393(0.5); 1.166(0.4); 1.149(0.4); 1.128(4.5); |
| | | | 1.111(10.2); 1.093(4.4); 1.086(0.6); 1.068(0.5); 1.064(1.0); 1.046(1.1); 1.027(16.0); |
| | | | 1.010(15.8); 0.146(0.4); 0.008(3.2); 0.000(107.4); −0.009(3.4); −0.150(0.5) |
| I-222 | 3.34 | 3.39 | Example I-222: ¹H-NMR(400.0 MHz, d₆-DMSO): δ = |
| | | | 11.001(6.9); 8.969(1.9); 8.954(4.0); 8.938(1.9); 8.543(8.8); 8.539(8.9); 8.313(2.9); |
| | | | 8.017(9.7); 8.014(9.6); 7.838(11.6); 7.834(6.8); 7.822(5.2); 7.818(13.2); 7.805(6.7); |
| | | | 7.787(3.2); 7.756(4.8); 7.736(2.5); 7.557(10.5); 7.537(9.1); 7.423(2.6); |
| | | | 7.287(6.6); 7.152(2.9); 4.658(7.4); 4.642(7.5); 4.039(0.7); 4.020(0.7); 3.317 |
| | | | (633.3); 2.675(2.8); 2.671(3.9); 2.666(2.8); 2.662(1.5); 2.648(0.3); 2.575(0.6); |
| | | | 2.524(8.9); 2.519(12.9); 2.510(208.5); 2.506(446.1); 2.501(629.4); 2.497(463.8); |
| | | | 2.493(218.3); 2.333(2.5); 2.328(3.5); 2.324(2.5); 2.320(1.2); 2.074(0.6); 2.057(1.6); |
| | | | 2.043(2.6); 2.027(1.7); 2.012(0.7); 1.988(3.1); 1.398(6.0); 1.234(0.4); |
| | | | 1.193(0.9); 1.175(1.7); 1.157(0.9); 0.874(16.0); 0.864(8.3); 0.855(7.2); 0.146 |
| | | | (1.9); 0.008(13.3); 0.000(444.8); −0.008(14.9); −0.150(1.9) |
| I-223 | 3.91 | 4.01 | Example I-223: ¹H-NMR(400.0 MHz, d₆-DMSO): δ = |
| | | | 9.936(6.5); 9.345(1.7); 9.330(3.5); 9.314(1.7); 8.579(7.6); 8.576(7.9); 8.039(8.1); |
| | | | 8.035(8.1); 7.942(8.8); 7.920(10.0); 7.828(8.9); 7.807(10.6); 7.793(0.9); |
| | | | 7.563(6.9); 7.547(12.4); 7.527(8.2); 7.495(0.7); 7.461(0.5); 7.457(0.5); 6.928(0.4); |
| | | | 6.924(0.4); 6.259(0.4); 4.637(6.4); 4.621(6.4); 4.590(0.5); 4.574(0.4); 4.080(1.2); |
| | | | 4.063(4.3); 4.047(4.4); 4.030(1.3); 3.385(40.4); 3.321(103.1); 2.677(0.5); |
| | | | 2.672(0.7); 2.668(0.5); 2.508(86.1); 2.503(117.6); 2.499(89.7); 2.334(0.5); 2.330(0.7); |
| | | | 1.374(16.0); 1.357(15.9); 0.146(0.5); 0.008(5.1); 0.000(115.4); −0.008(5.5); −0.150(0.6) |
| I-224 | 3.82 | 3.86 | Example I-224: ¹H-NMR(400.0 MHz, d₆-DMSO): δ = |
| | | | 11.026(6.3); 8.979(1.7); 8.963(3.7); 8.947(1.7); 8.557(8.4); 8.553(8.6); 8.314(1.4); |
| | | | 8.039(9.2); 8.035(9.2); 7.986(3.0); 7.970(2.1); 7.950(4.7); 7.930(2.6); 7.839(9.9); |
| | | | 7.835(3.8); 7.823(3.9); 7.818(13.0); 7.792(2.8); 7.558(9.6); 7.537(8.4); |
| | | | 5.754(5.2); 4.660(6.8); 4.644(6.8); 3.568(5.6); 3.318(253.6); 3.294(0.4); 2.675(1.3); |
| | | | 2.671(1.8); 2.666(1.3); 2.524(4.0); 2.520(5.8); 2.511(98.1); 2.506(213.5); |
| | | | 2.502(303.1); 2.497(223.3); 2.493(104.3); 2.338(0.6); 2.333(1.2); 2.329(1.8); |
| | | | 2.324(1.3); 2.320(0.6); 2.076(0.5); 2.060(1.5); 2.053(1.1); 2.046(2.4); 2.029 |
| | | | (1.7); 2.015(0.6); 0.877(16.0); 0.865(7.7); 0.858(7.1); 0.146(1.1); 0.008(8.1); |
| | | | 0.000(277.2); −0.009(8.6); −0.150(1.1) |
| I-225 | 4.34 | 4.40 | Example I-225: ¹H-NMR(400.0 MHz, d₆-DMSO): δ = |
| | | | 10.993(6.7); 8.962(1.9); 8.947(3.9); 8.930(1.9); 8.559(8.6); 8.555(8.9); 8.314(0.5); |
| | | | 8.010(9.4); 8.006(9.6); 7.978(9.6); 7.974(3.6); 7.962(3.4); 7.957(11.8); 7.951(1.4); |
| | | | 7.840(10.6); 7.823(3.8); 7.819(12.9); 7.714(9.0); 7.693(7.9); 7.560(10.3); |
| | | | 7.540(9.1); 5.754(7.4); 4.661(7.2); 4.645(7.2); 4.039(0.5); 4.021(0.5); 3.320(177.8); |
| | | | 2.676(0.8); 2.672(1.2); 2.667(0.9); 2.525(2.7); 2.520(3.9); 2.511(63.0); |
| | | | 2.507(135.6); 2.502(192.3); 2.498(142.2); 2.493(66.8); 2.334(0.8); 2.329(1.1); |
| | | | 2.325(0.8); 2.320(0.4); 2.077(0.6); 2.060(1.6); 2.052(1.1); 2.046(2.5); 2.029(1.8); |
| | | | 2.015(0.7); 1.989(2.2); 1.398(1.7); 1.259(0.4); 1.234(0.9); 1.194(0.6); |
| | | | 1.176(1.2); 1.158(0.7); 1.070(1.7); 0.873(16.0); 0.861(8.2); 0.854(7.2); 0.146(0.7); |
| | | | 0.008(5.2); 0.000(169.6); −0.008(5.5); −0.150(0.7) |
| I-226 | | 4.64 | Example I-226: ¹H-NMR(400.0 MHz, d₆-DMSO): δ = |
| | | | 10.939(6.6); 8.951(1.7); 8.936(3.7); 8.920(1.8); 8.520(7.4); 8.516(8.5); 7.976(8.3); |
| | | | 7.973(9.2); 7.914(9.3); 7.892(11.0); 7.794(3.1); 7.776(3.1); 7.761(1.5); 7.748(1.7); |
| | | | 7.740(2.2); 7.727(1.7); 7.543(7.1); 7.523(6.7); 7.502(3.0); 7.480(2.2); |
| | | | 4.620(6.8); 4.604(6.7); 3.321(308.1); 3.285(0.3); 2.995(0.4); 2.671(1.7); 2.570(0.4); |
| | | | 2.542(66.2); 2.506(201.5); 2.502(290.1); 2.498(230.6); 2.329(1.6); 2.324(1.4); |
| | | | 2.074(16.0); 2.050(1.6); 2.037(2.5); 2.021(1.7); 2.007(0.7); 1.335(0.4); |
| | | | 1.298(0.4); 1.258(0.5); 1.234(1.1); 0.867(14.6); 0.857(8.0); 0.848(7.0); 0.000 |
| | | | (8.0) |
| I-227 | | 4.23 | Example I-227: ¹H-NMR(400.0 MHz, d₆-DMSO): δ = |
| | | | 10.984(0.4); 8.529(0.5); 7.994(0.5); 7.989(0.5); 7.917(0.5); 7.895(0.7); 7.547(0.4); |
| | | | 7.526(0.4); 7.373(1.3); 7.361(0.9); 4.580(0.5); 4.564(0.5); 3.318(327.0); 2.670(1.7); |
| | | | 2.540(5.2); 2.523(3.6); 2.519(5.4); 2.510(87.9); 2.505(195.1); 2.501 |
| | | | (282.9); 2.496(222.0); 2.492(112.7); 2.332(1.2); 2.328(1.6); 2.323(1.3); |
| | | | 2.073(16.0); 1.259(0.4); 1.235(0.9); 0.859(0.9); 0.000(7.7) |
| I-228 | | 4.28 | Example I-228: ¹H-NMR(400.0 MHz, d₆-DMSO): δ = |
| | | | 11.026(0.4); 8.539(0.5); 8.535(0.5); 8.238(0.7); 8.215(0.8); 7.923(0.6); 7.919(0.6); |
| | | | 7.901(0.7); 7.879(0.8); 7.713(0.7); 7.692(0.6); 7.535(0.4); 7.516(0.4); 3.320(283.5); |
| | | | 2.674(0.9); 2.670(1.2); 2.666(0.9); 2.541(3.7); 2.524(2.6); 2.519(3.8); |
| | | | 2.510(65.7); 2.506(142.9); 2.501(204.1); 2.496(150.4); 2.492(70.2); 2.332(0.8); |
| | | | 2.328(1.2); 2.323(0.9); 2.073(16.0); 1.589(0.8); 1.571(0.8); 1.334(0.3); 1.249(0.4); |
| | | | 1.236(0.7); 0.882(0.9); 0.866(0.6); 0.000(4.3) |
| I-229 | | 4.23 | Example I-229: ¹H-NMR(400.0 MHz, d₆-DMSO): δ = |
| | | | 11.000(1.3); 9.182(1.4); 8.549(1.6); 8.546(1.7); 8.166(2.2); 8.144(2.3); 7.944(1.8); |
| | | | 7.941(1.9); 7.919(2.0); 7.913(0.7); 7.902(0.7); 7.897(2.3); 7.547(1.4); 7.527(1.2); |
| | | | 7.459(2.2); 7.442(0.6); 7.437(2.1); 3.362(0.4); 3.324(202.4); 2.676(0.5); |
| | | | 2.671(0.8); 2.666(0.5); 2.541(20.9); 2.524(1.7); 2.520(2.5); 2.511(42.5); |
| | | | 2.506(91.6); 2.502(129.9); 2.497(95.9); 2.493(45.0); 2.333(0.5); 2.328(0.8); |
| | | | 2.324(0.6); 2.073(16.0); 2.060(0.3); 2.053(0.5); 2.037(0.4); 1.507(2.1); 1.502(2.2); |
| | | | 1.235(0.5); 0.883(3.2); 0.871(1.7); 0.866(1.5); 0.000(1.5) |
| I-230 | 4.24 | 4.32 | Example I-230: ¹H-NMR(400.0 MHz, d₆-DMSO): δ = |
| | | | 10.997(7.0); 8.968(1.9); 8.952(4.0); 8.936(1.9); 8.511(8.5); 8.508(8.4); 8.314(0.7); |
| | | | 8.100(7.6); 8.095(7.6); 8.003(8.9); 8.000(8.6); 7.881(3.4); 7.876(3.1); 7.860(4.5); |
| | | | 7.854(4.4); 7.839(10.1); 7.819(11.6); 7.742(4.0); 7.739(4.0); 7.720(3.1); |
| | | | 7.558(10.4); 7.537(9.0); 4.658(7.7); 4.642(7.6); 3.321(148.3); 2.672(1.0); |
| | | | 2.507(117.9); 2.503(157.7); 2.499(118.4); 2.330(0.9); 2.075(1.8); 2.057(1.7); |
| | | | 2.042(2.6); 2.027(1.8); 2.012(0.7); 0.875(16.0); 0.864(8.7); 0.856(7.5); 0.832(0.4); |
| | | | 0.146(0.5); 0.008(4.3); 0.000(99.7); −0.008(3.7); −0.149(0.5) |
| I-231 | 4.43 | 4.41 | Example I-231: ¹H-NMR(400.0 MHz, d₆-DMSO): δ = |
| | | | 11.044(2.9); 10.581(0.4); 9.248(0.6); 8.560(3.4); 8.557(3.5); 8.271(0.4); 8.267 |
| | | | (0.4); 8.198(0.4); 8.194(0.4); 8.110(0.4); 8.106(0.4); 8.086(0.5); 8.064(0.5); |
| | | | 8.041(0.3); 8.019(0.5); 7.942(0.6); 7.935(0.6); 7.927(4.3); 7.922(1.8); 7.910(1.7); |
| | | | 7.905(4.8); 7.898(0.6); 7.892(0.4); 7.876(4.2); 7.855(5.4); 7.844(0.6); 7.833(0.8); |
| | | | 7.823(0.5); 7.809(0.4); 7.788(0.6); 7.763(3.8); 7.759(3.8); 7.731(0.5); 7.727(0.5); |
| | | | 7.631(0.5); 7.608(4.3); 7.587(3.7); 7.568(0.7); 7.543(3.5); 7.523(2.9); |
| | | | 7.444(0.4); 7.424(0.4); 5.754(0.8); 5.078(6.1); 5.062(1.0); 5.040(0.4); 4.802(0.7); |
| | | | 3.918(0.8); 3.815(1.6); 3.732(2.9); 3.697(1.0); 3.538(16.0); 3.524(1.9); 3.478(0.5); |
| | | | 3.452(1.6); 3.318(113.6); 2.676(0.6); 2.671(0.8); 2.667(0.6); 2.524(2.2); |
| | | | 2.511(45.3); 2.507(93.9); 2.502(130.4); 2.498(98.2); 2.494(48.2); 2.333(0.5); |
| | | | 2.329(0.7); 2.325(0.6); 2.119(1.5); 2.106(1.1); 2.093(0.7); 2.089(0.7); |
| | | | 1.398(0.4); 1.235(1.0); 1.051(0.4); 1.045(0.3); 1.030(0.3); 1.025(0.3); 0.994(0.3); |
| | | | 0.986(0.3); 0.872(5.2); 0.864(3.7); 0.853(2.9); 0.146(0.4); 0.008(3.0); |
| | | | 0.000(87.7); −0.008(3.3); −0.150(0.4) |
| I-232 | 4.31 | 4.30 | Example I-232: ¹H-NMR(400.0 MHz, d₆-DMSO): δ = |
| | | | 8.198(3.5); 8.194(3.6); 8.110(3.7); 8.106(3.4); 8.087(4.1); 8.065(4.4); 7.844(4.0); |
| | | | 7.824(4.7); 7.568(4.3); 7.556(3.3); 7.548(4.1); 7.536(2.9); 5.079(0.3); 5.058 |
| | | | (5.8); 3.733(16.0); 3.539(0.5); 3.452(15.1); 3.318(81.9); 2.807(0.6); 2.799(0.7); |
| | | | 2.787(1.1); 2.776(0.7); 2.768(0.6); 2.676(0.5); 2.671(0.6); 2.667(0.4); 2.524(1.5); |
| | | | 2.506(72.9); 2.502(98.8); 2.498(72.7); 2.333(0.4); 2.329(0.6); 2.325(0.4); |
| | | | 1.398(15.0); 1.070(0.5); 1.058(1.1); 1.050(2.2); 1.043(1.5); 1.030(2.1); |
| | | | 1.012(1.3); 1.006(2.0); 0.999(2.4); 0.995(2.3); 0.987(1.4); 0.974(0.4); 0.008(2.1); |
| | | | 0.000(65.3); −0.008(2.1) |
| I-233 | 3.91 | 3.90 | Example I-233: ¹H-NMR(400.0 MHz, d₆-DMSO): δ = |
| | | | 9.453(0.7); 9.438(1.5); 9.422(0.7); 8.356(3.4); 8.352(3.5); 8.109(3.6); 8.105(3.5); |
| | | | 8.057(0.4); 8.052(0.6); 8.045(3.9); 8.040(1.5); 8.028(1.4); 8.023(4.3); 7.820 |
| | | | (0.3); 7.806(4.0); 7.785(4.7); 7.553(2.8); 7.526(4.5); 7.505(3.3); 5.753(5.0); |
| | | | 4.887(0.4); 4.578(2.5); 4.562(2.5); 3.922(0.9); 3.728(16.0); 3.715(1.2); 3.319(109.5); |
| | | | 2.842(0.6); 2.836(0.6); 2.832(0.7); 2.823(1.1); 2.816(0.5); 2.811(0.6); 2.805(0.6); |
| | | | 2.675(0.4); 2.671(0.5); 2.666(0.4); 2.524(1.3); 2.511(30.3); 2.506(63.8); |
| | | | 2.502(89.0); 2.497(66.1); 2.493(31.6); 2.333(0.4); 2.329(0.5); 2.324(0.4); |
| | | | 1.235(0.6); 1.062(0.3); 1.048(1.0); 1.041(2.4); 1.021(4.8); 1.009(2.9); |
| | | | 1.002(1.1); 0.008(2.0); 0.000(63.7); −0.008(2.1) |
| I-234 | 2.89 | 2.97 | Example I-234: ¹H-NMR(400.0 MHz, d₆-DMSO): δ = |
| | | | 11.6070(3.1); 10.9371(2.6); 8.5054(3.4); 8.5024(3.5); 7.9152(4.0); 7.8934(4.6); |
| | | | 7.8545(3.2); 7.5433(3.4); 7.5225(3.1); 5.7550(4.8); 3.7427(16.0); 3.3244(19.4); |
| | | | 2.5078(29.9); 2.5036(39.0); 2.4994(29.8); 2.1010(0.8); 2.0872(1.2); |
| | | | 2.0712(0.8); 1.3977(0.4); 1.3106(0.3); 0.8666(7.4); 0.8557(4.2); 0.8474(3.6); |
| | | | 0.0076(0.8); −0.0002(19.7) |
| I-235 | 3.14 | 3.21 | Example I-235: ¹H-NMR(400.0 MHz, d₆-DMSO): δ = |
| | | | 11.4564(7.2); 10.9340(5.5); 8.5013(7.2); 8.4984(6.9); 8.4122(0.5); 8.3140(1.1); |
| | | | 7.9104(8.4); 7.8886(9.3); 7.8417(6.7); 7.5444(7.1); 7.5233(6.3); 5.7540(1.7); |
| | | | 3.9973(2.6); 3.9797(7.9); 3.9621(8.0); 3.9445(2.6); 3.3185(145.3); 3.2953(0.7); |
| | | | 2.6709(2.2); 2.5799(0.4); 2.5655(0.4); 2.5608(0.4); 2.5059(262.6); 2.5018(323.8); |
| | | | 2.4979(242.1); 2.3287(2.0); 2.1121(0.6); 2.0959(1.6); 2.0814(2.4); 2.0660(1.8); |
| | | | 2.0504(0.7); 1.3075(2.3); 1.2762(0.6); 1.2487(8.1); 1.2311(16.0); 1.2136(7.5); |
| | | | 0.8652(15.9); 0.8539(9.1); 0.8472(7.8); 0.1461(1.1); −0.0002(232.6); −0.1495(1.2) |
| I-236 | 3.50 | 3.59 | Example I-236: ¹H-NMR(400.0 MHz, d₆-DMSO): δ = |
| | | | 11.4250(4.9); 10.9299(4.0); 8.4995(5.3); 8.4970(5.3); 8.3140(1.6); 7.9065(6.3); |
| | | | 7.8847(7.1); 7.8370(5.0); 7.5447(5.4); 7.5238(4.9); 3.7609(7.0); 3.7429(7.0); |
| | | | 3.3361(1.5); 3.3172(80.8); 3.2933(0.8); 2.6706(1.8); 2.5055(219.7); 2.5015(280.1); |
| | | | 2.4974(212.7); 2.3280(1.8); 2.1061(0.5); 2.0911(1.3); 2.0761(1.8); |
| | | | 2.0609(1.4); 2.0457(0.5); 1.3980(16.0); 1.3052(0.3); 1.1595(0.5); 1.1474(1.0); |
| | | | 1.1386(0.9); 1.1287(1.4); 1.1170(1.0); 1.1093(1.0); 1.0975(0.6); 0.8652(12.4); |
| | | | 0.8535(7.0); 0.8470(6.4); 0.5773(1.2); 0.5660(3.7); 0.5624(4.0); 0.5462(3.7); |
| | | | 0.5427(3.6); 0.5320(1.3); 0.3146(1.4); 0.3030(4.7); 0.2894(4.5); 0.2777(1.2); |
| | | | 0.1459(0.9); 0.0140(4.1); 0.0071(9.7); −0.0002(188.8); −0.1496(1.0) |
| I-237 | 3.45 | 3.50 | Example I-237: ¹H-NMR(400.0 MHz, d₆-DMSO): δ = |
| | | | 11.2258(2.6); 10.9345(2.4); 8.5036(3.4); 8.4999(3.5); 7.9170(0.5); 7.9096(4.4); |
| | | | 7.9046(1.6); 7.8928(1.6); 7.8876(5.1); 7.8804(0.6); 7.8348(3.1); 7.8313(3.1); |
| | | | 7.5457(3.3); 7.5257(2.9); 5.7545(1.4); 4.2118(0.4); 4.1964(1.1); 4.1809(1.5); |
| | | | 4.1654(1.2); 4.1500(0.4); 3.3230(65.8); 2.6717(0.4); 2.5251(1.0); 2.5203(1.6); |
| | | | 2.5117(25.5); 2.5072(54.6); 2.5027(73.8); 2.4981(52.8); 2.4935(24.9); |
| | | | 2.3294(0.4); 2.0897(0.7); 2.0825(0.5); 2.0757(1.1); 2.0597(0.8); 1.3976(0.6); |
| | | | 1.2392(16.0); 1.2238(15.8); 1.2037(0.4); 0.8684(7.5); 0.8569(3.9); |
| | | | 0.8489(3.5); −0.0002(9.6); −0.0084(0.4) |
| I-238 | | 3.13 | Example I-238: ¹H-NMR(400.0 MHz, d₆-DMSO): δ = |
| | | | 11.6127(3.3); 10.9332(4.7); 8.5056(7.9); 8.5019(8.1); 8.3137(0.6); 7.9221(1.2); |
| | | | 7.9148(9.9); 7.9098(3.7); 7.8979(3.7); 7.8928(11.5); 7.8856(1.5); 7.8619(5.9); |
| | | | 7.8591(5.9); 7.5453(7.4); 7.5253(6.6); 4.0028(4.1); 3.9880(8.2); 3.9732(4.2); |
| | | | 3.3254(97.2); 2.7360(4.5); 2.7180(9.8); 2.7000(4.9); 2.6815(0.4); 2.6770(0.6); |
| | | | 2.6723(0.8); 2.6678(0.6); 2.5256(1.7); 2.5209(2.8); 2.5123(42.1); 2.5078(89.7); |
| | | | 2.5032(120.9); 2.4986(86.7); 2.4941(41.0); 2.3346(0.5); 2.3300(0.7); 2.3253(0.5); |
| | | | 2.1161(0.6); 2.0994(1.6); 2.0926(1.2); 2.0857(2.6); 2.0691(1.7); 2.0544(0.7); |
| | | | 1.9635(1.2); 1.9464(3.5); 1.9307(5.6); 1.9148(3.4); 1.8976(1.2); 1.3975(1.6); |
| | | | 0.8676(16.0); 0.8566(8.6); 0.8480(7.4); 0.8253(0.4); |
| | | | 0.0080(0.8); −0.0002(24.2); − 0.0085(0.8) |
| I-239 | 3.13 | 3.72 | Example I-239: ¹H-NMR(400.0 MHz, d₆-DMSO): δ = |
| | | | 12.0266(0.5); 12.0058(0.4); 11.3238(0.4); 11.2948(1.5); 8.4562(1.4); 8.3979(0.5); |
| | | | 8.3775(0.4); 8.3130(3.4); 8.2844(0.4); 8.2809(0.4); 8.2567(0.5); 8.2478(0.5); |
| | | | 8.2413(0.5); 8.2097(3.3); 8.1781(0.6); 8.1350(0.5); 8.1029(1.3); 8.0861(1.5); |
| | | | 8.0530(0.8); 8.0436(0.8); 8.0080(0.7); 8.0009(0.7); 7.9348(4.0); 7.9128(6.6); |
| | | | 7.8896(3.4); 7.8437(0.5); 7.7816(3.6); 7.7597(3.9); 7.7332(0.8); 7.7034(3.8); |
| | | | 7.6835(4.1); 7.6441(0.8); 7.6248(0.8); 7.5900(3.1); 7.5633(8.6); 7.5425(7.1); |
| | | | 7.5039(0.6); 7.4709(0.5); 7.4507(4.0); 7.4288(4.0); 7.3766(0.6); 7.3379(3.3); |
| | | | 7.3179(3.0); 7.2657(0.4); 7.2548(0.4); 7.0823(0.9); 7.0601(0.9); 6.5886(1.1); |
| | | | 6.5711(1.1); 3.9869(0.4); 3.7103(0.4); 3.6208(0.4); 3.5867(0.4); 3.5690(0.4); |
| | | | 3.5437(0.4); 3.4870(0.6); 3.4543(0.6); 3.4297(0.7); 3.3971(1.4); 3.3192(1190.0); |
| | | | 2.9808(0.4); 2.9436(16.0); 2.7276(0.4); 2.7114(0.4); 2.6750(6.8); 2.6706(9.3); |
| | | | 2.6661(6.9); 2.6299(0.5); 2.6021(0.7); 2.5406(4.2); 2.5239(21.7); 2.5190(33.6); |
| | | | 2.5104(548.2); 2.5060(1169.5); 2.5015(1582.1); 2.4970(1140.9); |
| | | | 2.4925(544.3); 2.3328(6.5); 2.3283(9.1); 2.3238(6.6); 2.1171(1.3); 2.1107(0.8); |
| | | | 2.0842(1.3); 2.0678(1.7); 2.0562(1.4); 2.0427(1.0); 2.0193(0.5); 1.9878(0.4); |
| | | | 1.3979(3.6); 1.2338(2.5); 1.1637(0.6); 1.1406(2.0); 1.0253(0.4); 1.0151(0.5); |
| | | | 0.9274(2.2); 0.9183(2.2); 0.8875(1.8); 0.8538(1.6); 0.8419(1.2); 0.7943(1.1); |
| | | | 0.7520(3.3); 0.6914(0.5); 0.6135(0.4); 0.5484(0.4); 0.5001(0.5); 0.4615(1.9); |
| | | | 0.3899(0.5); 0.3568(0.5); 0.3449(0.5); 0.1465(0.7); |
| | | | 0.0079(4.5); −0.0002(154.7); −0.0085(5.6); − 0.1503(0.7) |
| I-240 | 4.04 | 4.10 | Example I-240: ¹H-NMR(400.0 MHz, d₆-DMSO): δ = |
| | | | 10.9767(1.1); 10.9620(6.4); 9.1427(1.8); 9.1271(3.8); 9.1115(1.8); 8.7335(0.4); |
| | | | 8.5297(9.4); 8.5260(9.5); 8.3126(0.7); 8.2867(0.7); 8.2762(0.4); 8.0260(8.4); |
| | | | 8.0155(8.6); 8.0002(0.6); 7.9879(9.4); 7.9841(9.4); 7.9721(0.5); 7.9664(1.4); |
| | | | 7.9626(1.3); 7.9313(1.2); 7.9240(10.3); 7.9191(3.4); 7.9068(5.0); 7.9020(12.1); |
| | | | 7.8948(1.4); 7.8846(1.7); 7.8758(0.3); 7.8262(0.4); 7.7184(0.4); 7.5866(0.4); |
| | | | 7.5656(0.5); 7.5455(7.6); 7.5251(6.9); 7.1986(6.2); 7.1881(5.9); 4.7769(7.5); |
| | | | 4.7614(7.4); 3.3241(469.6); 3.3019(1.8); 2.9950(0.5); 2.7116(0.5); 2.6761(1.2); |
| | | | 2.6717(1.5); 2.6673(1.1); 2.5420(100.6); 2.5250(3.8); 2.5113(89.9); 2.5071(186.8); |
| | | | 2.5026(259.2); 2.4982(192.2); 2.4939(91.8); 2.3682(0.4); 2.3340(1.1); |
| | | | 2.3296(1.5); 2.3249(1.1); 2.0875(0.6); 2.0705(1.6); 2.0568(2.8); 2.0425(2.0); |
| | | | 2.0260(0.9); 0.9156(0.4); 0.8983(1.1); 0.8736(16.0); 0.8628(9.4); 0.8537(7.4); |
| | | | 0.1460(0.8); 0.0079(5.4); −0.0002(176.6); −0.0085(5.8); −0.1497(0.8) |
| I-241 | 3.96 | 3.90 | Example I-241: ¹H-NMR(400.0 MHz, d₆-DMSO): δ = |
| | | | 11.0528(6.9); 8.9825(2.0); 8.9679(3.6); 8.9530(2.0); 8.5135(3.0); 8.3145(0.4); |
| | | | 8.0482(12.3); 8.0445(12.0); 7.9527(13.0); 7.9307(14.7); 7.8329(13.3); |
| | | | 7.8122(15.7); 7.5610(11.0); 7.5508(16.0); 7.5407(11.1); 7.5304(13.2); 7.5137(1.0); |
| | | | 7.4921(0.6); 7.4595(0.4); 4.6494(10.5); 4.6337(10.5); 4.5895(0.4); 4.5714(0.3); |
| | | | 3.7501(1.4); 3.7261(3.4); 3.6987(3.4); 3.6727(1.4); 3.3217(120.1); 2.6720(1.4); |
| | | | 2.5071(182.0); 2.5027(241.8); 2.4985(185.0); 2.3297(1.4); 2.0869(0.4); −0.0002(21.4) |
| I-242 | 4.63 | 4.57 | Example I-242: ¹H-NMR(400.0 MHz, d₆-DMSO): δ = |
| | | | 10.0995(9.4); 9.1459(2.5); 9.1300(5.4); 9.1140(2.6); 8.4134(12.2); 8.4097(12.5); |
| | | | 8.3147(0.4); 8.0709(12.6); 8.0672(12.5); 7.9540(13.4); 7.9320(15.3); 7.8338(13.4); |
| | | | 7.8131(15.9); 7.5562(11.2); 7.5479(16.0); 7.5343(11.0); 7.5273(13.4); |
| | | | 4.6625(10.0); 4.6467(10.0); 3.3215(72.6); 2.6764(0.8); 2.6718(1.1); 2.6675(0.8); |
| | | | 2.5072(133.2); 2.5028(179.9); 2.4984(138.7); 2.3339(0.8); 2.3297(1.1); |
| | | | 2.3251(0.8); 1.6106(2.2); 1.5943(9.4); 1.5378(0.3); 1.4597(0.6); 1.4202(5.3); |
| | | | 1.4068(10.7); 1.4018(11.2); 1.3871(3.7); 1.1698(0.3); −0.0004(17.4) |
| I-243 | | 3.94 | Example I-243: ¹H-NMR(400.0 MHz, d₆-DMSO): δ = |
| | | | 11.0231(5.9); 8.5393(7.9); 8.5363(8.1); 8.3762(0.4); 8.3311(3.6); 8.3201(3.7); |
| | | | 8.3141(3.0); 7.9814(10.0); 7.9782(9.5); 7.9534(2.0); 7.9201(1.4); 7.9014(2.8); |
| | | | 7.8852(2.4); 7.8430(4.5); 7.8232(2.6); 7.3654(0.3); 7.3282(0.4); 4.1728(1.4); |
| | | | 3.5976(1.9); 3.3550(2.3); 3.3177(451.8); 2.9454(0.4); 2.9349(1.1); 2.9255(1.6); |
| | | | 2.9172(2.5); 2.9073(2.5); 2.8985(1.6); 2.8880(1.2); 2.8782(0.6); 2.6705(4.5); |
| | | | 2.6668(3.6); 2.5879(0.7); 2.5055(564.6); 2.5013(749.2); 2.4973(579.2); 2.3322(3.3); |
| | | | 2.3280(4.2); 2.0620(0.6); 2.0468(2.2); 2.0322(2.9); 2.0163(2.5); 2.0006(0.7); |
| | | | 1.3980(13.2); 1.2340(0.5); 1.1039(0.5); 0.8757(15.4); 0.8602(16.0); |
| | | | 0.8164(0.4); 0.8062(1.6); 0.7882(6.1); 0.7757(5.8); 0.7708(4.8); 0.7588(2.0); |
| | | | 0.7512(0.4); 0.7379(0.3); 0.7200(0.4); 0.6700(0.3); 0.6410(2.0); 0.6297(6.0); |
| | | | 0.6232(5.7); 0.6141(5.1); 0.6019(1.4); −0.0005(41.1); −0.0222(0.3) |
| I-244 | 4.08 | 4.08 | Example I-244: ¹H-NMR(400.0 MHz, d₆-DMSO): δ = |
| | | | 11.3929(0.7); 11.0817(4.2); 10.6619(0.4); 9.1147(5.0); 8.8364(0.4); 8.6902(0.6); |
| | | | 8.5766(5.9); 8.5729(6.1); 8.3133(2.6); 8.2474(0.5); 8.2251(0.4); 8.1510(0.5); |
| | | | 8.1287(0.4); 8.0931(0.4); 8.0194(6.5); 8.0159(6.6); 7.9980(1.7); 7.9723(1.7); |
| | | | 7.9275(1.4); 7.9074(2.3); 7.8900(1.9); 7.8509(3.2); 7.8285(1.9); 5.5878(0.7); |
| | | | 4.2816(0.3); 3.7452(0.4); 3.4609(0.4); 3.3173(841.4); 3.2848(31.4); 2.6748(5.2); |
| | | | 2.6703(6.9); 2.6658(5.3); 2.6161(0.7); 2.6019(0.8); 2.5235(19.6); 2.5101(407.9); |
| | | | 2.5057(835.2); 2.5012(1147.0); 2.4967(867.6); 2.4925(433.2); 2.3325(4.9); |
| | | | 2.3279(6.6); 2.3237(4.9); 2.0600(0.5); 2.0440(1.6); 2.0288(1.8); 2.0132(1.8); |
| | | | 1.9979(0.5); 1.3980(16.0); 1.2371(0.4); 1.1355(1.9); 1.1159(5.5); 1.1048(3.3); |
| | | | 1.0681(0.7); 1.0639(0.6); 1.0460(0.5); 1.0040(2.8); 0.9918(5.2); 0.9865(5.1); |
| | | | 0.9720(1.7); 0.9120(0.9); 0.8882(9.1); 0.8729(12.7); 0.8314(0.4); 0.1455(0.4); |
| | | | 0.0078(2.8); −0.0003(73.5); −0.0081(2.7); −0.1496(0.4) |
| I-245 | | 3.94 | Example I-245: ¹H-NMR(400.0 MHz, d₆-DMSO): δ = |
| | | | 11.0751(3.7); 9.0885(2.2); 9.0690(2.2); 8.5799(4.9); 8.5762(5.1); 8.0390(5.4); |
| | | | 8.0351(5.4); 8.0194(1.3); 7.9917(1.2); 7.9293(0.8); 7.9092(1.8); 7.8928(1.7); |
| | | | 7.8684(2.7); 7.8484(1.3); 5.1129(0.4); 5.0950(1.7); 5.0765(2.4); 5.0579(1.7); |
| | | | 5.0400(0.4); 3.3191(106.3); 2.6757(0.7); 2.6712(0.9); 2.6668(0.7); 2.5414(6.8); |
| | | | 2.5244(2.5); 2.5110(53.8); 2.5066(110.1); 2.5021(151.0); 2.4976(114.2); |
| | | | 2.4934(57.2); 2.3333(0.6); 2.3290(0.9); 2.3244(0.6); 2.1165(0.4); 2.1014(1.2); |
| | | | 2.0858(1.6); 2.0704(1.4); 2.0553(0.4); 1.9887(0.4); 1.6056(9.7); 1.5877(9.7); |
| | | | 1.3980(16.0); 0.8839(10.0); 0.8686(7.4); 0.0080(0.3); −0.0002(9.7); −0.0084(0.4) |
| I-246 | 4.32 | 4.37 | Example I-246: ¹H-NMR(400.0 MHz, d₆-DMSO): δ = |
| | | | 11.0409(5.7); 8.5721(2.0); 8.5527(9.2); 8.5489(8.7); 8.3137(3.3); 8.0521(0.5); |
| | | | 8.0260(8.7); 8.0222(8.4); 7.9889(2.2); 7.9621(2.1); 7.9273(1.4); 7.9064(3.1); |
| | | | 7.8896(2.4); 7.8506(4.2); 7.8297(2.5); 3.6411(2.2); 3.6246(5.8); 3.6082(6.1); |
| | | | 3.5916(2.4); 3.3177(561.8); 3.0248(0.4); 2.7136(0.5); 2.6750(6.5); 2.6705(8.7); |
| | | | 2.6661(7.0); 2.6501(1.8); 2.6391(2.6); 2.6217(4.3); 2.6110(3.0); 2.6044(2.9); |
| | | | 2.5931(4.6); 2.5755(3.1); 2.5647(2.9); 2.5103(523.8); 2.5060(1040.6); |
| | | | 2.5015(1411.4); 2.4971(1077.5); 2.3328(6.1); 2.3283(8.2); 2.3236(6.0); 2.0882(2.1); |
| | | | 2.0728(2.8); 2.0573(2.3); 2.0422(0.8); 1.3984(8.5); 1.2347(0.5); 1.1053(0.8); |
| | | | 0.9166(0.6); 0.8823(16.0); 0.8671(13.8); 0.0081(2.2); −0.0001(50.6); −3.7153(0.4) |
| I-247 | 4.49 | 4.56 | Example I-247: ¹H-NMR(400.0 MHz, d₆-DMSO): δ = |
| | | | 11.0838(2.4); 8.7753(1.4); 8.7557(1.4); 8.5682(3.2); 8.5646(2.7); 8.3139(0.3); |
| | | | 7.9871(4.1); 7.9835(3.7); 7.9553(0.9); 7.9159(0.6); 7.8965(1.2); 7.8796(1.0); |
| | | | 7.8431(4.8); 7.8227(4.8); 7.6404(3.8); 7.6199(3.0); 5.2578(0.8); 5.2402(1.1); |
| | | | 5.2211(0.8); 3.3184(61.9); 2.6750(0.8); 2.6707(0.9); 2.5059(122.1); 2.5018(141.5); |
| | | | 2.4975(103.2); 2.3324(0.7); 2.3287(0.8); 2.0780(0.8); 2.0631(1.0); 2.0474(0.8); |
| | | | 1.9888(0.3); 1.5657(4.6); 1.5481(4.5); 1.3979(16.0); 0.8880(5.6); |
| | | | 0.8727(4.8); −0.0001(3.0) |
| I-248 | | 4.55 | Example I-248: ¹H-NMR(400.0 MHz, d₆-DMSO): δ = |
| | | | 11.0286(1.6); 8.7736(0.4); 8.7592(0.9); 8.7445(0.5); 8.5524(2.0); 8.5486(2.1); |
| | | | 8.0086(2.2); 8.0047(2.2); 7.9770(0.6); 7.9495(0.5); 7.9174(0.4); 7.8979(0.7); |
| | | | 7.8807(0.7); 7.8339(3.0); 7.8165(2.6); 4.6886(1.6); 4.6743(1.6); 3.3208(20.9); |
| | | | 2.5256(0.6); 2.5122(13.5); 2.5078(28.2); 2.5032(39.4); 2.4988(29.8); 2.4945 |
| | | | (14.8); 2.0562(0.5); 2.0406(0.6); 2.0253(0.6); 1.9895(0.4); 1.3977(16.0); |
| | | | 0.8724(4.0); 0.8570(2.9); −0.0002(0.5) |
| I-249 | 4.51 | 4.51 | Example I-249: ¹H-NMR(400.0 MHz, d₆-DMSO): δ = |
| | | | 11.0419(2.2); 8.9801(0.7); 8.9651(1.3); 8.9498(0.6); 8.5677(3.0); 8.5639(3.0); |
| | | | 8.3140(1.2); 8.0396(3.3); 8.0357(3.3); 8.0244(1.1); 8.0113(1.2); 8.0012(1.7); |
| | | | 7.9885(1.6); 7.9681(0.8); 7.9275(0.6); 7.9075(1.2); 7.8906(1.1); 7.8665(0.3); |
| | | | 7.8547(1.8); 7.8350(0.9); 7.5557(1.0); 7.5413(1.0); 7.5322(1.0); 7.5177(0.9); |
| | | | 4.6663(2.3); 4.6510(2.3); 3.3188(64.4); 3.2955(0.5); 2.6755(0.7); 2.6711(0.9); |
| | | | 2.6664(0.7); 2.5242(2.8); 2.5108(54.4); 2.5064(109.7); 2.5019(149.3); |
| | | | 2.4974(112.5); 2.4931(55.7); 2.3333(0.6); 2.3289(0.9); 2.3242(0.6); 2.0810(0.7); |
| | | | 2.0654(0.9); 2.0502(0.8); 1.3979(16.0); 0.8813(6.0); 0.8661(3.8); |
| | | | 0.0078(1.6); −0.0003(40.3); −0.0085(1.6) |
| I-250 | 4.50 | 4.56 | Example I-250: ¹H-NMR(400.0 MHz, d₆-DMSO): δ = |
| | | | 11.0266(6.0); 9.0865(1.6); 9.0707(3.6); 9.0547(1.7); 8.5650(7.8); 8.5613(8.1); |
| | | | 8.3143(1.3); 8.0444(8.6); 8.0405(8.7); 8.0004(2.0); 7.9727(1.9); 7.9294(1.3); |
| | | | 7.9091(2.7); 7.8921(2.5); 7.8586(4.2); 7.8359(2.2); 7.4199(8.5); 7.3972(8.4); |
| | | | 5.7547(7.6); 4.6685(6.5); 4.6528(6.6); 3.3200(198.4); 3.0614(0.4); 2.6759(1.4); |
| | | | 2.6713(2.0); 2.6668(1.5); 2.5244(6.1); 2.5111(115.7); 2.5067(235.8); |
| | | | 2.5021(323.3); 2.4976(244.5); 2.4933(121.9); 2.3334(1.4); 2.3291(1.9); |
| | | | 2.3243(1.4); 2.1020(0.5); 2.0859(1.8); 2.0709(2.4); 2.0553(2.0); 2.0396(0.6); |
| | | | 1.9888(0.6); 1.3984(0.3); 1.2339(0.7); 1.1753(0.4); 0.8830(16.0); |
| | | | 0.8685(9.8); −0.0001(7.0) |
| I-251 | 4.38 | 4.45 | Example I-251: ¹H-NMR(400.0 MHz, d₆-DMSO): δ = |
| | | | 11.0517(1.8); 8.9369(0.5); 8.9214(1.1); 8.9057(0.5); 8.5692(2.4); 8.5654(2.5); |
| | | | 8.3145(0.6); 8.0411(2.7); 8.0372(2.7); 7.9949(0.7); 7.9685(0.7); 7.9259(0.4); |
| | | | 7.9058(0.9); 7.8895(1.9); 7.8649(1.2); 7.8615(1.3); 7.8536(1.3); 7.8306(0.7); |
| | | | 7.7170(1.0); 7.7134(1.0); 7.6971(1.4); 7.6935(1.4); 7.6078(0.9); 7.5886(1.5); |
| | | | 7.5692(0.7); 4.6828(1.9); 4.6673(1.9); 3.3199(22.1); 2.6716(0.4); 2.5248(1.2); |
| | | | 2.5114(22.7); 2.5070(45.9); 2.5024(62.7); 2.4979(47.3); 2.4936(23.5); |
| | | | 2.3293(0.4); 2.0709(0.5); 2.0557(0.7); 2.0405(0.6); 1.3977(16.0); 0.8793(5.0); |
| | | | 0.8656(2.9); −0.0002(1.4) |
| I-252 | 3.83 | 3.88 | Example I-252: ¹H-NMR(400.0 MHz, d₆-DMSO): δ = |
| | | | 11.9481(2.4); 11.0679(1.9); 8.5727(2.7); 8.5691(2.9); 8.5507(3.8); 8.3138(0.6); |
| | | | 8.0162(3.0); 8.0124(3.0); 7.9533(3.8); 7.9426(16.0); 7.9315(4.1); 7.9240(0.9); |
| | | | 7.5605(2.4); 7.5402(2.2); 3.3177(83.4); 2.6752(1.0); 2.6706(1.3); 2.6662(1.0); |
| | | | 2.5240(4.3); 2.5105(77.8); 2.5061(158.5); 2.5016(217.7); 2.4971(166.8); |
| | | | 2.4928(84.4); 2.3330(0.9); 2.3285(1.3); 2.3239(0.9); 2.1006(0.6); 2.0862(0.8); |
| | | | 2.0701(0.6); 1.2972(0.4); 1.2590(0.5); 1.2346(1.1); 0.8924(5.5); 0.8803(3.6); |
| | | | 0.8753(3.0); 0.8540(0.5); 0.1460(0.5); |
| | | | 0.0080(4.9); −0.0001(127.4); −0.0084(5.2); −0.1496(0.5) |
| I-253 | 3.91 | 3.96 | Example I-253: ¹H-NMR(400.0 MHz, d₆-DMSO): δ = |
| | | | 11.1270(2.0); 8.5789(2.7); 8.5751(2.8); 8.1614(2.6); 8.0220(3.1); 8.0181(3.1); |
| | | | 7.9785(0.8); 7.9519(0.8); 7.9264(0.5); 7.9063(1.1); 7.8887(0.9); 7.8396(1.4); |
| | | | 7.8170(0.8); 5.7546(1.9); 3.8569(4.5); 3.3191(30.4); 2.9612(12.3); |
| | | | 2.6715(0.4); 2.5247(1.2); 2.5113(23.9); 2.5069(49.1); 2.5023(67.6); 2.4978(51.1); |
| | | | 2.4934(25.2); 2.3292(0.4); 2.0637(0.7); 2.0489(0.9); 2.0330(0.7); 1.6278(16.0); |
| | | | 0.8913(4.4); 0.8761(5.4); 0.0080(0.6); −0.0002(16.2); −0.0085(0.6) |
| I-254 | 3.98 | 4.03 | Example I-254: ¹H-NMR(400.0 MHz, d₆-DMSO): δ = |
| | | | 10.9606(5.1); 8.9496(1.5); 8.9334(3.1); 8.9173(1.5); 8.5343(6.2); 8.5306(6.4); |
| | | | 8.3148(1.3); 7.9804(6.9); 7.9766(6.9); 7.8981(7.6); 7.8763(8.5); 7.8388(7.7); |
| | | | 7.8182(9.2); 7.5566(7.9); 7.5359(6.9); 7.5119(0.4); 7.4509(6.2); 7.4294(5.8); |
| | | | 6.5562(0.5); 6.5402(0.9); 6.5252(1.0); 6.5102(0.6); 6.4525(0.4); 6.4374(0.8); |
| | | | 6.4235(0.9); 6.4062(0.5); 4.6569(5.6); 4.6411(5.5); 3.3191(135.0); |
| | | | 2.6750(3.4); 2.6705(4.6); 2.6661(3.5); 2.5238(10.6); 2.5101(259.7); |
| | | | 2.5059(552.6); 2.5014(776.1); 2.4970(582.9); 2.4929(284.8); 2.4235(0.3); 2.3328(3.3); |
| | | | 2.3282(4.5); 2.3240(3.3); 2.0709(0.5); 2.0534(1.3); 2.0398(2.0); 2.0246(1.4); |
| | | | 2.0092(0.6); 1.9890(0.3); 1.3980(16.0); 0.8686(10.0); 0.8594(6.4); 0.8487(5.2); |
| | | | 0.0080(1.1); −0.0002(33.4); −0.0085(1.2) |
| I-255 | 3.88 | 3.90 | Example I-255: ¹H-NMR(400.0 MHz, d₆-DMSO): δ = |
| | | | 10.9620(6.4); 10.9404(0.3); 9.0695(1.8); 9.0541(3.7); 9.0387(1.8); 8.5253(8.2); |
| | | | 8.5215(8.5); 8.3147(14.1); 7.9754(9.1); 7.9715(9.3); 7.9188(10.2); 7.9019(3.6); |
| | | | 7.8970(11.7); 7.8499(8.5); 7.8404(8.8); 7.5463(7.2); 7.5261(6.3); 7.2366 |
| | | | (6.3); 7.2271(6.0); 4.7870(7.2); 4.7714(7.2); 3.5156(0.3); 3.3949(0.4); |
| | | | 3.3188(374.5); 3.2950(6.0); 2.8559(0.3); 2.7741(0.3); 2.7478(0.4); 2.6751(5.1); |
| | | | 2.6707(7.0); 2.6662(5.3); 2.5240(19.0); 2.5104(398.1); 2.5060(839.7); |
| | | | 2.5015(1178.2); 2.4971(888.0); 2.4928(433.5); 2.4400(0.4); 2.3329(4.8); 2.3283(6.6); |
| | | | 2.3240(4.8); 2.0790(0.6); 2.0623(1.6); 2.0476(2.4); 2.0317(1.7); 2.0163(0.7); |
| | | | 1.3980(2.9); 1.3417(0.6); 1.2349(0.6); 0.8700(16.0); 0.8584(8.2); 0.8508(7.3); |
| | | | 0.8124(0.3); 0.1462(3.6); |
| | | | 0.0081(29.2); −0.0001(837.3); −0.0082(31.7); −0.0353(0.6); −0.1433(0.3); −0.1496(3.6) |
| I-256 | 4.20 | 4.25 | Example I-256: ¹H-NMR(400.0 MHz, d₆-DMSO): δ = |
| | | | 11.2267(6.8); 9.0596(1.7); 9.0440(3.6); 9.0255(7.8); 9.0192(6.8); 8.6075(8.6); |
| | | | 8.6039(8.6); 8.4345(4.2); 8.4282(4.0); 8.4136(4.4); 8.4073(4.4); 8.3126(1.6); |
| | | | 8.1045(9.0); 8.1008(8.9); 7.9917(10.0); 7.9869(3.5); 7.9698(11.2); 7.8389(9.9); |
| | | | 7.8182(11.7); 7.7482(6.6); 7.7271(6.3); 7.5810(7.3); 7.5617(16.0); 7.5414(8.6); |
| | | | 4.6763(7.0); 4.6606(7.0); 3.4047(1.0); 3.3291(404.7); 2.6763(1.3); 2.6721(1.8); |
| | | | 2.6675(1.3); 2.5643(1.0); 2.5073(219.1); 2.5029(298.4); 2.4985(223.4); |
| | | | 2.3338(1.2); 2.3298(1.7); 2.3254(1.2); 1.3563(0.3); 1.2323(1.5); 1.1667(0.6); |
| | | | 0.0078(1.0); −0.0001(21.1); −0.0082(0.9) |
| I-257 | | 4.72 | Example I-257: ¹H-NMR(400.0 MHz, d₆-DMSO): δ = |
| | | | 12.0217(8.2); 11.0761(6.4); 8.5763(8.2); 8.5727(8.4); 8.5505(9.0); 8.3151(1.2); |
| | | | 8.0153(8.8); 8.0115(8.9); 7.9548(9.8); 7.9379(3.7); 7.9329(11.4); 7.9256(3.3); |
| | | | 7.9054(4.2); 7.8865(2.7); 7.8470(3.5); 7.8169(7.6); 7.7948(3.0); 7.5620(7.3); |
| | | | 7.5417(6.6); 7.5201(0.4); 3.3196(88.2); 2.6758(1.9); 2.6711(2.7); 2.6667(2.0); |
| | | | 2.6065(0.3); 2.5244(6.9); 2.5195(10.4); 2.5110(149.9); 2.5065(319.0); 2.5020(448.7); |
| | | | 2.4975(334.2); 2.4932(159.5); 2.3332(1.8); 2.3289(2.5); 2.3243(1.8); |
| | | | 2.3200(0.9); 2.1193(0.6); 2.1047(1.6); 2.0891(2.5); 2.0734(1.8); 2.0581(0.7); |
| | | | 1.3516(0.3); 1.2980(0.4); 1.2595(0.6); 1.2338(2.0); 0.8933(16.0); 0.8810 |
| | | | (9.2); 0.8748(8.0); 0.1460(1.7); |
| | | | 0.0080(12.4); −0.0002(394.1); −0.0084(13.8); −0.1497(1.7) |
| I-258 | 4.24 | 2.08 | Example I-258: ¹H-NMR(400.0 MHz, d₆-DMSO): δ = |
| | | | 11.0718(4.4); 8.5755(5.5); 8.2137(6.1); 7.9527(7.3); 7.9307(8.3); 7.9146(1.0); |
| | | | 7.8887(7.9); 7.8680(9.6); 7.8357(0.4); 7.8166(0.3); 7.7963(0.4); 7.6569(8.8); |
| | | | 7.6363(7.5); 7.5944(6.5); 7.5740(5.8); 7.5500(0.6); 7.3618(0.3); 5.7572(3.3); |
| | | | 4.7674(5.4); 4.5501(1.2); 4.0909(1.3); 3.5723(0.8); 3.4668(0.7); 3.3210(116.2); |
| | | | 2.6752(1.8); 2.6707(2.6); 2.6661(1.9); 2.5241(6.2); 2.5193(9.3); 2.5106(149.2); |
| | | | 2.5061(317.6); 2.5016(427.9); 2.4970(309.4); 2.4926(149.9); 2.3328(1.7); |
| | | | 2.3284(2.5); 2.3238(1.8); 2.1170(3.2); 2.0997(0.6); 2.0855(2.4); 2.0688(2.5); |
| | | | 2.0536(2.2); 2.0380(0.7); 1.9694(0.6); 1.9085(0.4); 1.5660(0.4); 1.2978(0.5); |
| | | | 1.2584(0.9); 1.2346(3.0); 1.1664(0.5); 1.1401(7.6); 0.8866(16.0); 0.8712(13.0); |
| | | | 0.8532(1.3); 0.1459(0.4); 0.0080(3.3); −0.0002(106.3); −0.0085(3.7); −0.1497(0.4) |
| I-259 | 3.58 | 3.55 | Example I-259: ¹H-NMR(400.0 MHz, d₆-DMSO): δ = |
| | | | 10.9963(7.6); 8.6795(2.1); 8.6642(4.6); 8.6488(2.2); 8.5321(9.5); 8.5284(10.0); |
| | | | 7.9892(10.4); 7.9853(10.7); 7.9161(11.2); 7.8991(4.3); 7.8941(12.9); 7.8230(0.4); |
| | | | 7.8151(0.4); 7.8083(0.4); 7.7965(0.4); 7.7917(0.4); 7.7834(0.5); 7.6320(11.1); |
| | | | 7.6105(13.3); 7.5911(0.4); 7.5461(8.5); 7.5258(7.7); 7.4772(0.5); 7.4655(0.8); |
| | | | 7.4613(0.8); 7.4218(0.5); 7.4166(0.5); 7.4085(0.4); 7.3801(11.6); 7.3585(10.2); |
| | | | 7.3448(0.9); 5.7575(2.9); 4.5463(8.6); 4.5311(8.6); 3.8364(6.5); |
| | | | 3.8189(11.0); 3.8012(6.8); 3.3228(38.5); 2.6756(0.9); 2.6713(1.2); 2.6670(1.0); |
| | | | 2.5245(3.8); 2.5067(160.5); 2.5023(206.4); 2.4979(152.2); 2.4662(8.1); |
| | | | 2.3335(0.9); 2.3289(1.2); 2.3246(0.9); 2.0958(1.8); 2.0864(1.4); 2.0775(4.9); |
| | | | 2.0586(7.1); 2.0396(5.4); 2.0207(3.4); 2.0075(3.1); 1.9904(2.0); 1.9767(0.8); |
| | | | 1.2312(1.3); 1.1663(0.6); 1.1490(0.4); 0.8590(16.0); 0.8501(10.5); 0.8391(8.2); |
| | | | 0.1460(0.7); 0.0078(6.2); −0.0002(157.3); −0.0083(7.7); −0.1499(0.7) |
| I-260 | 4.66 | 4.68 | Example I-260: ¹H-NMR(400.0 MHz, d₆-DMSO): δ = |
| | | | 10.9947(6.6); 8.9676(1.9); 8.9522(3.9); 8.9361(1.9); 8.5238(5.0); 8.3163(0.4); |
| | | | 8.0085(9.4); 8.0047(8.9); 7.9155(9.8); 7.9105(3.6); 7.8934(11.1); 7.8407(9.7); |
| | | | 7.8198(11.4); 7.7184(6.7); 7.6982(9.1); 7.5938(8.4); 7.5738(6.4); 7.5571(9.9); |
| | | | 7.5369(15.2); 7.5173(6.7); 5.7563(5.4); 4.6581(7.1); 4.6423(7.1); 3.9280(12.0); |
| | | | 3.3278(73.6); 2.6764(0.9); 2.6721(1.2); 2.6677(1.0); 2.5074(172.7); |
| | | | 2.5030(222.6); 2.4985(158.3); 2.4944(75.7); 2.3343(1.0); 2.3297(1.3); |
| | | | 2.3253(0.9); 2.0869(1.0); 2.0753(16.0); 1.2337(0.5); 0.1458(1.0); |
| | | | 0.0079(9.3); −0.0001(226.4); −0.0084(9.8); −0.1497(1.0) |
| I-261 | 4.19 | 2.09 | Example I-261: ¹H-NMR(400.0 MHz, d₆-DMSO): δ = |
| | | | 20.0107(0.4); 11.3824(0.6); 11.0864(0.5); 11.0223(3.3); 8.6827(0.9); 8.5370(4.7); |
| | | | 8.3157(1.1); 8.1874(4.3); 8.1364(1.1); 8.0537(0.5); 8.0334(0.8); 7.9712(0.5); |
| | | | 7.9613(0.8); 7.9408(1.7); 7.9145(6.0); 7.8928(6.9); 7.8147(3.4); 7.7935(3.5); |
| | | | 7.7577(0.5); 7.7139(0.5); 7.6518(0.5); 7.6120(0.5); 7.5476(6.6); 7.5273(5.7); |
| | | | 7.4770(0.8); 7.4294(0.6); 7.4155(0.7); 7.3622(0.9); 7.3444(0.9); 7.3230(0.7); |
| | | | 7.3024(0.6); 7.2915(0.6); 7.2783(0.8); 7.2610(1.0); 7.2510(1.0); 7.1971(1.4); |
| | | | 7.1359(1.8); 7.0603(0.9); 7.0394(0.8); 6.9992(0.6); 6.9753(0.6); 6.9713(0.6); |
| | | | 6.9301(0.8); 6.8883(0.7); 6.8776(0.6); 6.8567(0.6); 6.8340(0.6); 6.7929(0.5); |
| | | | 3.3217(253.3); 2.6711(4.9); 2.6523(0.5); 2.5974(0.5); 2.5063(662.5); |
| | | | 2.5020(848.2); 2.4980(617.5); 2.3291(4.7); 2.0364(1.7); 2.0202(2.2); |
| | | | 2.0046(1.8); 1.2342(1.8); 0.9225(0.6); 0.8892(16.0); 0.8769(9.4); 0.8211(0.5); |
| | | | 0.1467(2.1); 0.0081(21.3); 0.0001(437.1); −0.1491(2.0) |
| I-262 | 3.73 | 3.75 | Example I-262: ¹H-NMR(400.0 MHz, d₆-DMSO): δ = |
| | | | 10.9838(6.3); 8.9607(1.7); 8.9452(3.7); 8.9296(1.8); 8.5153(7.8); 8.5117(7.9); |
| | | | 7.9939(8.3); 7.9902(8.1); 7.8989(2.9); 7.8791(4.8); 7.8596(3.5); 7.8388(12.0); |
| | | | 7.8177(12.4); 7.8105(4.6); 7.5982(0.5); 7.5829(3.6); 7.5780(3.5); 7.5547(10.9); |
| | | | 7.5337(8.5); 7.4141(0.4); 7.3973(0.4); 7.3576(0.4); 7.3407(0.5); 4.6542(7.3); |
| | | | 4.6386(7.2); 3.3237(83.2); 2.6716(1.0); 2.5069(141.3); 2.5026(182.9); |
| | | | 2.4983(133.4); 2.3293(1.0); 2.0751(1.5); 2.0694(0.6); 2.0521(1.7); 2.0379(2.6); |
| | | | 2.0225(1.8); 2.0072(0.7); 0.8707(16.0); 0.8595(8.9); 0.8518(7.8); 0.1459(0.5); |
| | | | 0.0077(5.1); −0.0001(106.4); −0.1493(0.5) |
| I-263 | 4.54 | 4.57 | Example I-263: ¹H-NMR(400.0 MHz, d₆-DMSO): δ = |
| | | | 11.5207(0.5); 11.5146(0.4); 11.5081(0.4); 11.4655(5.0); 11.2742(0.4); |
| | | | 11.2452(0.3); 11.2180(5.8); 11.1900(0.3); 10.7254(0.4); 8.7876(0.4); 8.7523(0.3); |
| | | | 8.6836(7.8); 8.6804(8.1); 8.4419(0.5); 8.4189(8.6); 8.3970(10.9); 8.3601(0.6); |
| | | | 8.3384(0.5); 8.3154(0.7); 8.2819(0.8); 8.2394(10.0); 8.2177(8.2); 8.1584(8.0); |
| | | | 8.1553(8.2); 8.1149(0.5); 8.0597(3.4); 8.0291(3.4); 7.9507(2.4); 7.9287(3.8); |
| | | | 7.9114(3.8); 7.8931(5.1); 7.8713(2.4); 7.8430(0.5); 5.7557(3.7); 4.0493(0.4); |
| | | | 4.0386(0.4); 4.0207(0.4); 3.7753(0.3); 3.4407(0.4); 3.4198(0.4); 3.3209(215.3); |
| | | | 3.2533(0.4); 2.8910(5.3); 2.7987(0.4); 2.7313(5.0); 2.6897(1.7); 2.6710(2.6); |
| | | | 2.5845(0.4); 2.5018(467.9); 2.4041(0.3); 2.3282(2.6); 2.1838(0.4); 2.0743(13.0); |
| | | | 2.0399(2.1); 2.0264(2.7); 2.0107(2.2); 1.9960(0.8); 1.4545(0.3); 1.3554 |
| | | | (1.2); 1.2324(2.6); 1.1814(1.4); 1.1677(1.3); 1.0460(1.8); 1.0305(1.7); |
| | | | 0.9824(0.5); 0.9684(0.4); 0.9219(12.1); 0.9069(16.0); 0.8697(0.8); 0.8537(0.9); |
| | | | 0.1461(1.4); 0.0506(0.4); −0.0003(298.3); −0.1500(1.4) |
| I-264 | 4.53 | 4.62 | Example I-264: ¹H-NMR(400.0 MHz, d₆-DMSO): δ = |
| | | | 11.2748(6.0); 10.7211(6.6); 8.6782(7.6); 8.6748(7.9); 8.3155(0.3); 8.1444(7.9); |
| | | | 8.1410(8.1); 8.0363(3.0); 8.0071(3.1); 7.9428(1.6); 7.9229(3.6); 7.9046(3.3); |
| | | | 7.8756(4.5); 7.8540(2.2); 5.7560(0.5); 3.3212(78.8); 2.8235(0.6); 2.8107(1.3); |
| | | | 2.8040(1.6); 2.7921(2.6); 2.7805(1.6); 2.7732(1.5); 2.7608(0.7); 2.6722(1.1); |
| | | | 2.5068(142.8); 2.5027(187.1); 2.4985(142.0); 2.3295(1.0); 2.0867(0.5); |
| | | | 2.0750(0.6); 2.0590(0.6); 2.0437(1.9); 2.0290(2.5); 2.0137(1.9); 1.9981(0.7); |
| | | | 1.9896(0.3); 1.2304(1.0); 1.1672(0.8); 1.1492(0.4); 1.0674(0.8); 1.0539(2.7); |
| | | | 1.0470(6.4); 1.0271(9.7); 1.0234(9.8); 1.0114(7.4); 0.9897(0.8); 0.9046(10.1); |
| | | | 0.8910(16.0); 0.1463(0.9); −0.0002(188.2); −0.1495(0.9) |
| I-265 | | 4.51 | Example I-265: ¹H-NMR(400.0 MHz, d₆-DMSO): δ = |
| | | | 11.0813(5.2); 8.9726(1.4); 8.9571(2.8); 8.9413(1.4); 8.2874(0.5); 8.1902(6.7); |
| | | | 8.1870(7.2); 8.0629(0.4); 8.0403(16.0); 7.8454(7.4); 7.8246(8.8); 7.7008(7.3); |
| | | | 7.6975(7.5); 7.5700(7.6); 7.5494(6.6); 4.6505(5.5); 4.6347(5.5); 3.3418(114.8); |
| | | | 2.7142(0.5); 2.6743(0.4); 2.5444(110.1); 2.5275(2.1); 2.5095(50.6); |
| | | | 2.5051(67.0); 2.5006(51.6); 2.3704(0.5); 2.3317(0.4); 2.0638(0.4); 2.0468(1.2); |
| | | | 2.0334(1.9); 2.0197(1.2); 2.0164(1.2); 2.0023(0.5); 0.8818(0.4); 0.8590(5.2); |
| | | | 0.8473(6.7); 0.8389(9.6); −0.0002(1.1) |
| I-266 | | 4.34 | Example I-266: ¹H-NMR(400.0 MHz, d₆-DMSO): δ = |
| | | | 11.0780(6.8); 8.9912(1.8); 8.9755(3.7); 8.9597(1.8); 8.3816(0.7); 8.3657(8.3); |
| | | | 8.3625(9.0); 7.8890(1.6); 7.8695(2.4); 7.8457(10.4); 7.8365(10.8); 7.8329(11.5); |
| | | | 7.8252(12.1); 7.6300(5.7); 7.6094(5.2); 7.5668(10.4); 7.5463(9.2); 4.6629(7.4); |
| | | | 4.6472(7.3); 3.3592(51.6); 2.7211(0.5); 2.5514(95.5); 2.5164(23.5); |
| | | | 2.5119(31.2); 2.5075(24.1); 2.3773(0.5); 2.0768(0.6); 2.0609(1.8); 2.0463(2.6); |
| | | | 2.0315(1.8); 2.0152(0.7); 0.8900(0.5); 0.8673(8.1); 0.8601(9.3); 0.8484(16.0) |
| I-267 | | 4.45 | Example I-267: ¹H-NMR(400.0 MHz, d₆-DMSO): δ = |
| | | | 11.0218(6.3); 8.9867(1.7); 8.9710(3.6); 8.9552(1.8); 8.5875(0.6); 8.5308(7.3); |
| | | | 8.5272(8.1); 8.0431(7.9); 8.0394(8.6); 7.8421(9.4); 7.8213(11.2); 7.7619(2.9); |
| | | | 7.7291(3.3); 7.7168(5.8); 7.5600(9.4); 7.5394(8.4); 4.6603(6.9); 4.6447(6.9); |
| | | | 3.3414(120.2); 2.6749(0.4); 2.6704(0.3); 2.5816(6.4); 2.5712(12.3); |
| | | | 2.5602(6.8); 2.5451(23.0); 2.5102(50.5); 2.5057(67.6); 2.5013(52.7); 2.3325(0.4); |
| | | | 2.0748(0.6); 2.0587(1.7); 2.0436(2.4); 2.0282(1.9); 2.0127(0.6); 0.8789(16.0); |
| | | | 0.8659(8.9); 0.8620(8.6); −0.0002(1.3) |
| I-268 | | 4.05 | Example I-268: ¹H-NMR(400.0 MHz, d₆-DMSO): δ = |
| | | | 11.0684(6.6); 8.9854(1.8); 8.9698(3.6); 8.9538(1.8); 8.3973(7.2); 7.8665(7.2); |
| | | | 7.8393(9.6); 7.8185(12.2); 7.8081(7.6); 7.7893(7.1); 7.5621(9.6); 7.5416(8.4); |
| | | | 4.6562(7.1); 4.6406(7.1); 3.3446(115.2); 2.7161(0.5); 2.6757(0.4); |
| | | | 2.5461(109.5); 2.5113(47.4); 2.5069(63.0); 2.5025(49.2); 2.3724(0.5); |
| | | | 2.3335(0.4); 2.0709(0.6); 2.0551(1.8); 2.0403(2.5); 2.0250(1.9); 2.0093(0.7); |
| | | | 0.8601(9.5); 0.8472(16.0); −0.0002(0.8) |
| I-269 | | 4.34 | Example I-269: ¹H-NMR(400.0 MHz, d₆-DMSO): δ = |
| | | | 11.0716(5.3); 8.9953(1.4); 8.9797(2.8); 8.9640(1.4); 8.3870(0.6); 8.3606(6.8); |
| | | | 8.0630(2.7); 8.0468(2.8); 7.8486(9.6); 7.8283(16.0); 7.8251(11.2); 7.8182(4.3); |
| | | | 7.5682(8.3); 7.5476(7.4); 4.6659(5.8); 4.6501(5.8); 3.3703(24.8); 2.7244(0.4); |
| | | | 2.5546(68.5); 2.5197(14.6); 2.5152(19.4); 2.5108(15.0); 2.3807(0.4); 2.0789(0.5); |
| | | | 2.0635(1.4); 2.0486(2.1); 2.0337(1.5); 2.0175(0.6); 0.8705(6.4); 0.8637(7.4); |
| | | | 0.8519(12.6) |
| I-270 | | 4.45 | Example I-270: ¹H-NMR(400.0 MHz, d₆-DMSO): δ = |
| | | | 11.0000(7.2); 8.9792(1.9); 8.9639(3.9); 8.9482(1.9); 8.5605(8.9); 8.5570(9.4); |
| | | | 8.0329(9.7); 8.0292(10.0); 7.8528(7.2); 7.8429(11.9); 7.8220(15.1); 7.7982(4.6); |
| | | | 7.7583(6.9); 7.7376(3.6); 7.5622(11.2); 7.5416(9.9); 4.6638(8.1); 4.6480 |
| | | | (8.0); 3.3477(159.6); 2.7153(0.7); 2.6798(0.4); 2.6752(0.5); 2.6707(0.4); |
| | | | 2.5558(17.5); 2.5454(139.7); 2.5147(31.8); 2.5105(62.2); 2.5060(82.3); 2.5015(62.7); |
| | | | 2.3715(0.6); 2.3373(0.4); 2.3327(0.5); 2.3282(0.4); 2.0766(0.8); 2.0587(1.8); |
| | | | 2.0522(1.4); 2.0451(2.8); 2.0285(1.9); 2.0139(0.8); 0.8782(16.0); 0.8681 |
| | | | (9.6); 0.8583(7.8); 0.8357(0.5); −0.0002(0.8) |
| I-271 | | 4.23 | Example I-271: ¹H-NMR(400.0 MHz, d₆-DMSO): δ = |
| | | | 11.0563(6.4); 9.0027(1.7); 8.9873(3.4); 8.9718(1.7); 8.6047(0.6); 8.5342(7.3); |
| | | | 8.5307(8.2); 8.0606(7.9); 8.0570(8.4); 7.8907(6.6); 7.8624(6.9); 7.8422(9.5); |
| | | | 7.8216(11.1); 7.5603(9.6); 7.5398(8.6); 4.6611(7.0); 4.6454(7.0); 3.3468(86.3); |
| | | | 2.7165(0.3); 2.5468(72.1); 2.5117(36.4); 2.5074(48.2); 2.5031(37.7); 2.3726(0.3); |
| | | | 2.0774(0.7); 2.0612(2.0); 2.0458(2.7); 2.0306(2.2); 2.0150(0.6); 0.8813(16.0); |
| | | | 0.8659(13.3); −0.0002(0.6) |
| I-272 | | 4.17 | Example I-272: ¹H-NMR(400.0 MHz, d₆-DMSO): δ = |
| | | | 11.0880(4.0); 9.0562(1.1); 9.0413(1.6); 9.0275(1.5); 8.5023(7.2); 7.9478(7.0); |
| | | | 7.8374(6.8); 7.8170(8.4); 7.7454(0.6); 7.7257(6.6); 7.7164(6.9); 7.6947(0.6); |
| | | | 7.5567(7.9); 7.5365(7.1); 4.6572(6.0); 4.6416(6.1); 3.5947(0.4); 3.4595(148.2); |
| | | | 3.4529(162.6); 3.4425(110.0); 3.4354(149.6); 3.4325(152.7); 3.4132(157.2); |
| | | | 2.7187(0.5); 2.6830(0.4); 2.6793(0.4); 2.5486(111.0); 2.5140(57.7); 2.5097(75.4); |
| | | | 2.5054(57.0); 2.3745(0.5); 2.3364(0.5); 2.0458(1.2); 2.0307(1.7); 2.0173(1.5); |
| | | | 0.8762(12.0); 0.8613(16.0) |
| I-273 | | 4.45 | Example I-273: ¹H-NMR(400.0 MHz, d₆-DMSO): δ = |
| | | | 11.0442(6.9); 8.9967(1.8); 8.9811(3.9); 8.9653(1.8); 8.5783(0.6); 8.5587(7.7); |
| | | | 8.5551(8.8); 8.1077(7.7); 8.0488(8.4); 8.0451(9.4); 7.9910(1.1); 7.9672(7.7); |
| | | | 7.9625(10.6); 7.9415(1.5); 7.8419(9.9); 7.8212(12.0); 7.5607(9.9); |
| | | | 7.5402(8.8); 4.6625(7.3); 4.6468(7.3); 3.3447(190.9); 2.7147(0.4); 2.6792(0.4); |
| | | | 2.6747(0.5); 2.6704(0.4); 2.5451(103.7); 2.5101(63.1); 2.5057(84.9); 2.5013(67.1); |
| | | | 2.3710(0.4); 2.3364(0.4); 2.3323(0.5); 2.3283(0.4); 2.0769(1.2); 2.0612(1.7); |
| | | | 2.0543(1.3); 2.0469(2.6); 2.0309(1.9); 2.0159(0.7); 0.8814(16.0); 0.8701(8.9); |
| | | | 0.8626(7.9); −0.0002(0.8) |
| I-274 | | 4.28 | Example I-274: ¹H-NMR(400.0 MHz, d₆-DMSO): δ = |
| | | | 11.0836(6.6); 8.9988(1.8); 8.9831(3.7); 8.9671(1.8); 8.4937(7.6); 7.9479(7.8); |
| | | | 7.8614(1.6); 7.8400(10.3); 7.8195(12.4); 7.6293(0.4); 7.5756(0.4); 7.5562(9.7); |
| | | | 7.5358(8.6); 4.6578(7.0); 4.6420(6.9); 3.3475(149.9); 2.9980(0.4); 2.7142(0.6); |
| | | | 2.6742(0.5); 2.5446(126.4); 2.5095(53.6); 2.5051(71.4); 2.5007(55.5); |
| | | | 2.3704(0.6); 2.3318(0.4); 2.3272(0.3); 2.0717(0.6); 2.0559(1.8); 2.0410(2.4); |
| | | | 2.0255(1.9); 2.0099(0.7); 0.8698(9.8); 0.8564(16.0); −0.0003(0.3) |
| I-275 | | 4.11 | Example I-275: ¹H-NMR(400.0 MHz, d₆-DMSO): δ = |
| | | | 11.0558(6.6); 8.9935(1.7); 8.9778(3.5); 8.9621(1.7); 8.4849(7.7); 7.9705(2.4); |
| | | | 7.9639(3.0); 7.9557(2.9); 7.9485(3.4); 7.9362(10.0); 7.8402(9.6); 7.8195(11.2); |
| | | | 7.5569(9.6); 7.5364(8.4); 4.6572(7.0); 4.6414(7.0); 3.3469(228.2); 3.3438(209.5); |
| | | | 2.6787(0.4); 2.6744(0.6); 2.6701(0.5); 2.5445(44.1); 2.5097(73.8); 2.5053(97.2); |
| | | | 2.5009(74.5); 2.3365(0.4); 2.3322(0.6); 2.3278(0.4); 2.0760(0.6); 2.0705(0.6); |
| | | | 2.0546(2.0); 2.0397(2.6); 2.0240(2.1); 2.0084(0.6); 0.8690(12.6); |
| | | | 0.8539(16.0); −0.0002(0.7) |
| I-276 | | 4.57 | Example I-276: ¹H-NMR(400.0 MHz, d₆-DMSO): δ = |
| | | | 11.1004(8.7); 8.9852(2.1); 8.9699(4.2); 8.9549(2.1); 8.3335(0.6); 8.2294(10.2); |
| | | | 8.2262(11.7); 8.1876(0.5); 8.1301(0.3); 8.1052(5.8); 8.0897(5.7); 7.8485(12.4); |
| | | | 7.8278(14.2); 7.7411(11.7); 7.7378(12.0); 7.7017(0.5); 7.6985(0.5); |
| | | | 7.5741(12.6); 7.5536(11.1); 4.6571(9.4); 4.6413(9.3); 3.3597(117.1); 3.3573(115.3); |
| | | | 2.7188(0.7); 2.5489(132.2); 2.5321(1.8); 2.5140(37.8); 2.5096(49.5); 2.5052(37.9); |
| | | | 2.3749(0.6); 2.0685(0.8); 2.0518(2.1); 2.0380(3.3); 2.0244(2.1); 2.0068(0.9); |
| | | | 0.8876(0.8); 0.8652(9.0); 0.8531(11.7); 0.8452(16.0); −0.0002(0.6) |
| I-277 | 3.33 | 2.17 | Example I-277: ¹H-NMR(400.0 MHz, d₆-DMSO): δ = |
| | | | 11.0447(5.9); 8.9682(1.7); 8.9524(3.4); 8.9365(1.7); 8.5695(7.3); 8.5659(7.6); |
| | | | 8.5380(8.5); 8.5344(6.0); 8.5267(5.9); 8.5231(8.8); 8.0528(7.8); 8.0491(7.8); |
| | | | 8.0063(2.0); 7.9797(1.9); 7.9304(1.2); 7.9099(2.6); 7.8935(2.4); 7.8623(4.1); |
| | | | 7.8396(2.2); 7.3519(7.9); 7.3373(7.8); 4.6065(6.9); 4.5908(6.8); 3.9256(0.9); |
| | | | 3.3208(180.7); 2.6752(1.7); 2.6709(2.3); 2.6666(1.8); 2.5062(286.9); 2.5018(374.6); |
| | | | 2.4974(280.5); 2.3330(1.5); 2.3286(2.1); 2.3240(1.6); 2.0831(0.6); 2.0673(1.7); |
| | | | 2.0523(2.3); 2.0367(1.9); 2.0219(0.6); 1.3513(0.4); 1.3363(0.5); 1.2980(1.3); |
| | | | 1.2587(2.0); 1.2344(3.5); 1.1746(0.4); 0.8796(16.0); 0.8659(10.6); 0.7947(0.3); |
| | | | 0.1460(0.7); 0.0078(6.8); −0.0002(154.1); −0.1495(0.7) |
| I-278 | 4.52 | 4.67 | Example I-278: ¹H-NMR(400.0 MHz, d₆-DMSO): δ = |
| | | | 11.0539(3.2); 8.9926(0.9); 8.9769(1.8); 8.9606(0.8); 8.5291(3.7); 8.5258(3.7); |
| | | | 8.0632(3.9); 8.0597(3.8); 8.0163(1.5); 7.9820(4.5); 7.8396(4.5); 7.8191(5.2); |
| | | | 7.8072(0.6); 7.5565(4.6); 7.5361(4.0); 4.6569(3.4); 4.6415(3.4); 4.6169(0.4); |
| | | | 4.6007(0.3); 3.3280(29.4); 2.6710(1.4); 2.5060(181.5); 2.5017(230.0); 2.4976 |
| | | | (169.2); 2.3331(1.0); 2.3284(1.4); 2.0602(1.0); 2.0445(1.2); 2.0291(1.1); |
| | | | 2.0131(0.4); 1.3979(16.0); 0.8790(8.2); 0.8639(5.9); 0.1460(0.9); |
| | | | 0.0073(9.8); −0.0002(183.3); −0.1495(0.9) |

The log P values are measured according to EEC Directive 79/831 Annex V.A8 by HPLC (high-performance liquid chromatography) on a reversed-phase column (C 18). Temperature: 55° C.

The LC-MS determination in the acidic range is effected at pH 2.7 using 0.1% aqueous formic acid and acetonitrile (contains 0.1% formic acid) as eluents; linear gradient from 10% acetonitrile to 95% acetonitrile. Called log P (HCOOH) in the table.

LC-MS determination in the neutral range is effected at pH 7.8 with 0.001 molar aqueous ammonium hydrogencarbonate solution and acetonitrile as eluents; linear gradient from 10% acetonitrile to 95% acetonitrile. Called log P (neutral) in the table.

Calibration is effected using unbranched alkan-2-ones (having 3 to 16 carbon atoms) with known log P values (log P values determined on the basis of the retention times by linear interpolation between two successive alkanones).

The NMR data of selected examples are listed either in conventional form (6 values, multiplet splitting, number of hydrogen atoms) or as NMR peak lists.

In each case, the solvent in which the NMR spectrum was recorded is stated.

### NMR Peak List Method

The ¹H NMR data of selected examples are stated in the form of ¹H NMR peak lists. For each signal peak, first the 6 value in ppm and then the signal intensity in round brackets are listed. The pairs of 6 value-signal intensity numbers for different signal peaks are listed with separation from one another by semicolons.

The peak list for one example therefore takes the form of:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of ¹H NMR spectra, we use tetramethylsilane and/or the chemical shift of the solvent, particularly in the case of spectra which are measured in DMSO. Therefore, the tetramethylsilane peak may but need not occur in NMR peak lists.

The lists of the ¹H NMR peaks are similar to the conventional ¹H NMR printouts and thus usually contain all peaks listed in a conventional NMR interpretation.

In addition, like conventional ¹H NMR printouts, they may show solvent signals, signals of stereoisomers of the target compounds which are likewise provided by the invention, and/or peaks of impurities.

In the reporting of compound signals within the delta range of solvents and/or water, our lists of ¹H NMR peaks show the standard solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%).

Such stereoisomers and/or impurities may be typical of the particular preparation process. Their peaks can thus help in this case to identify reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the peak picking in question in conventional ¹H NMR interpretation.

Further details of ¹H NMR peak lists can be found in the Research Disclosure Database Number 564025.

### USE EXAMPLES

Ctenocephalides felis—In Vitro Contact Tests with Adult Cat Fleas

For the coating of the test tubes, 9 mg of active ingredient are first dissolved in 1 ml of acetone p.a. and then diluted to the desired concentration with acetone p.a. 250 μl of the solution are distributed homogeneously on the inner walls and the base of a 25 ml glass tube by turning and rocking on an orbital shaker (rocking rotation at 30 rpm for 2 h). With 900 ppm of active ingredient solution and internal surface area 44.7 cm², given homogeneous distribution, an area-based dose of 5 μg/cm² is achieved.

After the solvent has evaporated off, the tubes are populated with 5-10 adult cat fleas (Ctenocephalides felis), sealed with a perforated plastic lid and incubated in a horizontal position at room temperature and ambient humidity. After 48 h, efficacy is determined. To this end, the tubes are stood upright and the fleas are knocked to the base of the tube. Fleas which remain motionless at the base or move in an uncoordinated manner are considered to be dead or moribund.

A substance shows good efficacy against Ctenocephalides felis if at least 80% efficacy was achieved in this test at an application rate of 5 μg/cm². 100% efficacy means that all the fleas were dead or moribund. 0% efficacy means that no fleas were harmed.

In this test, for example, the following compounds from the preparation examples show an efficacy of 100% at an application rate of 5 μg/cm² (500 g/ha): I-118, I-128, I-138, I-162, I-197, 216.

In this test, for example, the following compounds from the preparation examples show an efficacy of 80% at an application rate of 5 μg/cm² (500 g/ha): I-013

Boophilus microplus—Injection Test

Solvent: dimethyl sulphoxide

To produce a suitable active ingredient formulation, 10 mg of active ingredient are mixed with 0.5 ml of solvent and the concentrate is diluted to the desired concentration with solvent.

1 μl of the active ingredient solution is injected into the abdomen of 5 engorged adult female cattle ticks (Boophilus microplus). The animals are transferred into dishes and kept in a climate-controlled room.

Efficacy is assessed after 7 days by laying of fertile eggs. Eggs which are not visibly fertile are stored in a climate-controlled cabinet until the larvae hatch after about 42 days. An efficacy of 100% means that none of the ticks has laid any fertile eggs; 0% means that all the eggs are fertile.

In this test, for example, the following compounds from the preparation examples show an efficacy of 95% at an application rate of 20 μg/animal: I-104.

In this test, for example, the following compounds from the preparation examples show an efficacy of 80% at an application rate of 20 μg/animal: I-151

Ctenocephalides felis—Oral Test

Solvent: dimethyl sulphoxide

To produce a suitable active ingredient formulation, 10 mg of active ingredient are mixed with 0.5 ml of dimethyl sulphoxide. Dilution with citrated cattle blood gives the desired concentration.

About 20 unfed adult cat fleas (Ctenocephalides felis) are placed into a chamber which is closed at the top and bottom with gauze. A metal cylinder whose bottom end is closed with parafilm is placed onto the chamber. The cylinder contains the blood/active ingredient formulation, which can be imbibed by the fleas through the parafilm membrane.

After 2 days, the kill in % is determined. 100% means that all of the fleas have been killed; 0% means that none of the fleas have been killed.

In this test, for example, the following compounds from the preparation examples show an efficacy of 100% at an application rate of 100 ppm: I-004, I-005, I-008, I-013, I-026, I-027, I-028, I-030, I-036, I-043, I-044, I-051, I-052, I-073, I-074, I-075, I-076, I-077, I-078, I-079, I-080, I-081, I-082, I-085, I-089, I-094, I-096, I-098, I-102, I-105, I-108, I-109, I-110, I-113, I-115, I-116, I-117, I-118, I-120, I-124, I-128, I-130, I-131, I-133, I-138, I-141, I-142, I-149, I-151, I-152, I-154, I-155, I-159, I-160, I-162, I-172, I-173, I-176, I-178, I-179, I-182, I-184, I-189, I-190, I-192, I-195, I-196, I-197, I-213, I-216, I-224, I-225, I-226, I-228, I-232, I-233, I-245, I-247, I-249, I-250.

In this test, for example, the following compounds from the preparation examples show an efficacy of 98% at an application rate of 100 ppm: I-015, I-017, I-025, I-040, I-070

In this test, for example, the following compounds from the preparation examples show an efficacy of 95% at an application rate of 100 ppm: I-041, I-053, I-065, I-091, I-093, I-095, I-150, I-235.

In this test, for example, the following compounds from the preparation examples show an efficacy of 90% at an application rate of 100 ppm: I-088, I-167.

In this test, for example, the following compounds from the preparation examples show an efficacy of 80% at an application rate of 100 ppm: I-104, I-217.

Lucilia cuprina Test

Solvent: dimethyl sulphoxide

To produce a suitable active ingredient formulation, 10 mg of active ingredient are mixed with 0.5 ml of dimethyl sulphoxide, and the concentrate is diluted with water to the desired concentration.

About 20 L1 larvae of the Australian sheep blowfly (Lucilia cuprina) are transferred into a test vessel containing minced horsemeat and the active ingredient formulation of the desired concentration.

After 2 days, the kill in % is determined. 100% means that all the larvae have been killed; 0% means that no larvae have been killed.

In this test, for example, the following compounds from the preparation examples show an efficacy of 100% at an application rate of 100 ppm: I-005, I-013, I-015, I-026, I-040, I-043, I-044, I-048, I-052, I-053, I-070, I-073, I-074, I-077, I-078, I-079, I-080, I-081, I-082, I-085, I-091, I-093, I-094, I-096, I-098, I-104, I-107, I-113, I-120, I-124, I-128, I-133, I-138, I-141, I-149, I-151, I-154, I-155, I-160, I-172, I-173, I-176, I-178, I-182, I-184, I-190, I-195, I-196, I-197, I-213, I-217, I-224, I-225, I-228, I-247, I-249.

In this test, for example, the following compounds from the preparation examples show an efficacy of 95% at an application rate of 100 ppm: I-008, I-089, I-117, I-250.

In this test, for example, the following compounds from the preparation examples show an efficacy of 90% at an application rate of 100 ppm: I-027, I-030, I-108, I-115, I-142, I-152, I-159, I-192.

In this test, for example, the following compounds from the preparation examples show an efficacy of 85% at an application rate of 100 ppm: 1-021.

In this test, for example, the following compounds from the preparation examples show an efficacy of 80% at an application rate of 100 ppm: 1-216, 1-245.

Musca domestica Test

Solvent: dimethyl sulphoxide

To produce a suitable active ingredient formulation, 10 mg of active ingredient are mixed with 0.5 ml of dimethyl sulphoxide, and the concentrate is diluted with water to the desired concentration.

Vessels containing a sponge treated with sugar solution and the active ingredient formulation of the desired concentration are populated with 10 adult houseflies (Musca domestica).

After 2 days, the kill in % is determined. 100% means that all of the flies have been killed; 0% means that none of the flies have been killed.

In this test, for example, the following compounds from the preparation examples show an efficacy of 100% at an application rate of 100 ppm: I-013, I-026, I-074, I-078, I-089, I-138, I-151, I-154, I-155, I-172, I-195.

In this test, for example, the following compounds from the preparation examples show an efficacy of 90% at an application rate of 100 ppm: I-040, I-079, I-107, I-152.

In this test, for example, the following compounds from the preparation examples show an efficacy of 80% at an application rate of 100 ppm: I-073, I-077, I-085, I-115, I-159, I-160, I-196, I-197.

In this test, for example, the following compounds from the preparation examples show an efficacy of 100% at an application rate of 20 ppm: 1-184, 1-195, 1-197.

Meloidogyne incognita Test

Solvent: 125.0 parts by weight of acetone

To produce a suitable active ingredient formulation, 1 part by weight of active ingredient is mixed with the stated amount of solvent and the concentrate is diluted with water to the desired concentration.

Vessels are filled with sand, active ingredient solution, an egg/larvae suspension of the southern root-knot nematode (Meloidogyne incognita) and lettuce seeds. The lettuce seeds germinate and the plants develop. The galls develop on the roots.

After 14 days, the nematicidal efficacy in % is determined by the formation of galls. 100% means that no galls were found; 0% means that the number of galls on the treated plants corresponds to the untreated control.

In this test, for example, the following compound from the preparation examples shows an efficacy of 100% at an application rate of 20 ppm: I-025

In this test, for example, the following compound from the preparation examples shows an efficacy of 90% at an application rate of 20 ppm: I-028, I-038, I-104, I-138, I-162, I-177, I-257, I-259.

Myzus persicae—Spray Test

Solvent: 78 parts by weight of acetone
1.5 parts by weight of dimethylformamide

Emulsifier: alkylaryl polyglycol ether

To produce a suitable active ingredient formulation, 1 part by weight of active ingredient is dissolved with the specified parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) infested by all stages of the green peach aphid (Myzus persicae) are sprayed with an active ingredient formulation of the desired concentration.

After 6 days, the efficacy in % is determined. 100% means that all the aphids have been killed; 0% means that no aphids have been killed.

In this test, for example, the following compounds from the preparation examples show an efficacy of 90% at an application rate of 500 g/ha: 1-028, 1-175.

Phaedon cochleariae—Spray Test

Solvent: 78.0 parts by weight of acetone
1.5 parts by weight of dimethylformamide

Emulsifier: alkylaryl polyglycol ether

To produce a suitable active ingredient formulation, 1 part by weight of active ingredient is dissolved with the specified parts by weight of solvent and made up with water containing an emulsifier concentration of 1000 ppm until the desired concentration is attained. To produce further test concentrations, the formulation is diluted with emulsifier-containing water.

Discs of Chinese cabbage leaves (Brassica pekinensis) are sprayed with an active ingredient formulation of the desired concentration and, after drying, populated with larvae of the mustard beetle (Phaedon cochleariae).

After 7 days, the efficacy in % is determined. 100% means that all the beetle larvae have been killed; 0% means that no beetle larvae have been killed.

In this test, for example, the following compounds from the preparation examples show an efficacy of 100% at an application rate of 500 g/ha: I-002, I-004, I-005, I-007, I-009, I-011, I-012, I-013, I-015, I-017, I-018, I-019, I-020, I-021, I-022, I-024, I-025, I-026, I-027, I-028, I-030, I-031, I-035, I-039, I-040, I-041, I-042, I-043, I-044, I-048, I-051, I-052, I-053, I-055, I-056, I-060, I-061, I-064, I-066, I-067, I-068, I-070, I-074, I-075, I-076, I-077, I-078, I-079, I-080, I-081, I-082, I-084, I-085, I-086, I-089, I-090, I-091, I-093, I-094, I-096, I-097, I-098, I-099, I-102, I-103, I-105, I-107, I-109, I-110, I-111, I-112, I-113, I-114, I-115, I-116, I-117, I-118, I-119, I-120, I-123, I-124, I-125, I-126, I-127, I-128, I-129, I-130, I-131, I-132, I-137, I-138, I-140, I-141, I-142, I-144, I-145, I-146, I-147, I-149, I-151, I-152, I-153, I-154, I-157, I-158, I-160, I-161, I-162, I-172, I-173, I-174, I-176, I-178, I-179, I-180, I-182, I-184, I-185, I-186, I-188, I-189, I-190, I-192, I-195, I-196, I-197, I-201, I-204, I-205, I-206, I-208, I-213, I-216, I-218, I-222, I-224, I-225, I-226, I-227, I-228, I-233, I-234, I-235, I-236, I-240, I-241, I-243, I-244, I-245, I-246, I-247, I-248, I-249, I-250, I-251, I-252, I-253, I-254, I-262, I-263, I-264, I-265, I-270, I-274, II-276

In this test, for example, the following compounds from the preparation examples show an efficacy of 83% at an application rate of 500 g/ha: I-016, I-037, I-038, I-045, I-050, I-057, I-065, I-069, I-071, I-087, I-095, II-101, I-108, I-135, I-155, I-191, I-194, I-210, I-217, I-237, I-238, I-258, I-259, I-261, I-268, I-271, I-272, I-277.

In this test, for example, the following compounds from the preparation examples show an efficacy of 100% at an application rate of 100 g/ha: I-005, I-011, I-012, I-013, I-026, I-027, I-030, I-038, I-039, I-040, I-041, I-042, I-044, I-045, I-051, I-052, I-068, I-074, I-076, I-080, I-082, I-085, I-089, I-093, I-094, I-096, I-098, I-099, I-102, I-105, I-106, I-107, I-110, I-111, I-115, I-116, I-118, I-119, I-120, I-124, I-128, I-129, I-130, I-137, I-138, I-140, I-141, I-142, I-144, I-145, I-147, I-149, I-151, I-152, I-153, I-154, I-160, I-162, I-173, I-174, I-176, I-178, I-180, I-182, I-184, I-185, I-188, I-189, I-190, I-192, I-195, I-196, I-204, I-208, I-209, I-213, I-215, I-216, I-218, I-222, I-224, I-225, I-243, I-244, I-245, I-247, I-248, I-249, I-250, I-253, I-254, I-262, I-264.

In this test, for example, the following compounds from the preparation examples show an efficacy of 83% at an application rate of 100 g/ha: I-015, I-022, I-077, I-081, I-158, I-161, I-179, I-197, I-210, I-214, I-230, I-239, I-246, I-251, I-268.

Spodoptera frugiperda—Spray Test

Solvent: 78.0 parts by weight of acetone
1.5 parts by weight of dimethylformamide

Emulsifier: alkylaryl polyglycol ether

To produce a suitable active ingredient formulation, 1 part by weight of active ingredient is dissolved with the specified parts by weight of solvent and made up with water containing an emulsifier concentration of 1000 ppm until the desired concentration is attained. To produce further test concentrations, the formulation is diluted with emulsifier-containing water.

Leaf discs of maize (Zea mays) are sprayed with an active ingredient formulation of the desired concentration and, after drying, populated with caterpillars of the armyworm (Spodoptera frugiperda).

After 7 days, the efficacy in % is determined. 100% means that all the caterpillars have been killed; 0% means that no caterpillar has been killed.

In this test, for example, the following compounds from the preparation examples show an efficacy of 100% at an application rate of 500 g/ha: I-004, I-005, I-006, I-008, I-009, I-010, I-011, I-012, I-013, I-015, I-016, I-017, I-018, I-019, I-020, I-021, I-022, I-025, I-026, I-027, I-028, I-030, I-031, I-032, I-033, I-034, I-035, I-037, I-039, I-040, I-041, I-042, I-043, I-044, I-045, I-048, I-051, I-052, I-053, I-054, I-055, I-056, I-057, I-058, I-059, I-060, I-061, I-062, I-063, I-064, I-065, I-066, I-067, I-068, I-071, I-073, I-074, I-075, I-076, I-077, I-078, I-079, I-080, I-081, I-082, I-083, I-085, I-087, I-089, I-090, I-091, I-092, I-093, I-094, I-095, I-096, I-098, I-099, I-102, I-103, I-105, I-106, I-107, I-109, I-110, I-111, I-112, I-113, I-114, I-115, I-116, I-117, I-118, I-119, I-120, I-121, I-122, I-123, I-124, I-125, I-126, I-127, I-128, I-129, I-130, I-131, I-132, I-133, I-134, I-135, I-136, I-137, I-138, I-139, I-140, I-141, I-144, I-145, I-147, I-148, I-149, I-150, I-151, I-152, I-153, I-154, I-155, I-156, I-158, I-159, I-160, I-161, I-162, I-163, I-164, I-169, I-170, I-172, I-173, I-174, I-176, I-178, I-181, I-182, I-184, I-185, I-189, I-190, I-191, I-192, I-193, I-195, I-196, I-197, I-198, I-200, I-204, I-205, I-206, I-207, I-211, I-212, I-214, I-216, I-217, I-218, I-220, I-223, I-224, I-225, I-226, I-227, I-228, I-229, I-231, I-232, I-233, I-237, I-240, I-241, I-242, I-243, I-244, I-245, I-246, I-247, I-248, I-249, I-250, I-251, I-252, I-253, I-254, I-255, I-256, I-257, I-259, I-260, I-261, I-262, I-263, I-264, I-265, I-268, I-270, I-271, I-273, I-274, I-277.

In this test, for example, the following compounds from the preparation examples show an efficacy of 83% at an application rate of 500 g/ha: I-003, I-007, I-023, I-029, I-036, I-038, I-046, I-047, I-049, I-070, I-086, I-088, I-100, I-108, I-146, I-157, I-166, I-179, I-187, I-215, I-219, I-239, I-258, I-269, I-276.

In this test, for example, the following compounds from the preparation examples show an efficacy of 80% at an application rate of 500 g/ha: I-014

In this test, for example, the following compounds from the preparation examples show an efficacy of 100% at an application rate of 100 g/ha: I-005, I-008, I-013, I-015, I-022, I-025, I-026, I-027, I-039, I-041, I-044, I-051, I-052, I-055, I-056, I-061, I-064, I-065, I-067, I-068, I-071, I-074, I-076, I-077, I-078, I-079, I-081, I-082, I-085, I-086, I-087, I-088, I-089, I-091, I-096, I-098, I-099, I-108, I-110, I-111, I-115, I-117, I-118, I-119, I-120, I-122, I-124, I-125, I-128, I-129, I-131, I-132, I-133, I-137, I-138, I-140, I-143, I-144, I-145, I-147, I-149, I-150, I-151, I-152, I-153, I-155, I-156, I-157, I-158, I-159, I-160, I-161, I-162, I-164, I-169, I-172, I-173, I-176, I-178, I-181, I-182, I-185, I-189, I-190, I-192, I-195, I-196, I-197, I-198, I-200, I-204, I-205, I-206, I-208, I-213, I-214, I-215, I-216, I-217, I-218, I-224, I-225, I-231, I-232, I-233, I-241, I-243, I-244, I-245, I-246, I-247, I-248, I-249, I-250, I-251, I-253, I-260, I-261, I-262, I-264, I-268, I-277.

In this test, for example, the following compounds from the preparation examples show an efficacy of 83% at an application rate of 100 g/ha: I-018, I-028, I-030, I-037, I-040, I-042, I-048, I-060, I-062, I-073, I-080, I-083, I-090, I-102, I-105, I-112, I-113, I-114, I-121, I-126, I-127, I-130, I-134, I-141, I-154, I-170, I-211, I-226, I-228, I-240, I-252, I-254, I-255.

In this test, for example, the following compounds from the preparation examples show an efficacy of 80% at an application rate of 100 g/ha: 1-106, 1-221.

Tetranychus urticae—Spray Test, OP-Resistant

Solvent: 78.0 parts by weight of acetone
1.5 parts by weight of dimethylformamide

Emulsifier: alkylaryl polyglycol ether

To produce a suitable active ingredient formulation, 1 part by weight of active ingredient is dissolved with the specified parts by weight of solvent and made up with water containing an emulsifier concentration of 1000 ppm until the desired concentration is attained. To produce further test concentrations, the formulation is diluted with emulsifier-containing water.

Discs of bean leaves (Phaseolus vulgaris) infested with all stages of the greenhouse red spider mite (Tetranychus urticae) are sprayed with an active ingredient formulation of the desired concentration.

After 6 days, the efficacy in % is determined. 100% means that all the spider mites have been killed; 0% means that no spider mites have been killed.

In this test, for example, the following compounds from the preparation examples show an efficacy of 100% at an application rate of 500 g/ha: I-105

In this test, for example, the following compounds from the preparation examples show an efficacy of 90% at an application rate of 500 g/ha: I-036, I-091, II-101, I-104, I-182, I-185, I-194, I-218, I-227, I-255.

In this test, for example, the following compounds from the preparation examples shows an efficacy of 90% at an application rate of 100 g/ha: I-100, I-194, I-218, I-227.

1. Compound of formula (I) and/or salt thereof 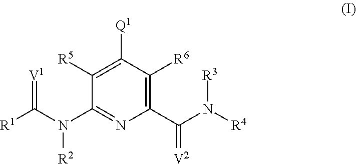
in which
Q¹ is in each case optionally singly or multiply, identically or differently substituted aryl, 1,3-benzodioxolyl, 2,3-dihydro-1,4-benzodioxinyl, hetaryl or oxohetaryl,
where the substituents are selected from: cyano, carboxyl, halogen, nitro, hydroxyl, amino, SCN, SF₅, tri-(C₁-C₆)alkylsilyl, (C₁-C₆)alkyl, (C₁-C₆)haloalkyl, (C₁-C₆)cyanoalkyl, (C₁-C₆)hydroxyalkyl, hydroxycarbonyl-(C₁-C₆)-alkoxy, (C₁-C₆)alkoxycarbonyl-(C₁-C₆)alkyl, (C₁-C₆)alkoxy-(C₁-C₆)alkyl, (C₂-C₆)alkenyl, (C₂-C₆)haloalkenyl, (C₂-C₆)cyanoalkenyl, (C₂-C₆)alkynyl, (C₂-C₆)haloalkynyl, (C₂-C₆)cyanoalkynyl, (C₁-C₆)alkoxy, (C₁-C₆)haloalkoxy, (C₁-C₆)cyanoalkoxy, (C₁-C₆)alkoxycarbonyl-(C₁-C₆)alkoxy, (C₁-C₆)alkoxy-(C₁-C₆)alkoxy, (C₃-C₈)cycloalkyl, (C₃-C₉)halocycloalkyl, (C₁-C₆)alkyl-(C₃-C₈)cycloalkyl, (C₁-C₆)haloalkyl-(C₃-C₈)cycloalkyl, (C₁-C₄)haloalkenoxy, (C₁-C₆)alkylhydroxyimino, (C₁-C₆)alkoxyimino, (C₁-C₆)alkyl-(C₁-C₆)alkoxyimino, (C₁-C₆)haloalkyl-(C₁-C₆)alkoxyimino, (C₁-C₆)alkylsulphanyl, (C₁-C₆)alkoxy-(C₁-C₆)alkylsulphanyl, (C₁-C₆)alkylsulphanyl-(C₁-C₆)alkyl, (C₁-C₆)alkylsulphinyl, (C₁-C₆)haloalkylsulphinyl, (C₁-C₆)alkoxy-(C₁-C₆)alkylsulphinyl, (C₁-C₆)alkylsulphinyl-(C₁-C₆)alkyl, (C₁-C₆)alkylsulphonyl, (C₁-C₆)haloalkylsulphonyl, (C₁-C₆)alkoxy-(C₁-C₆)alkylsulphonyl, (C₁-C₆)alkylsulphonyl-(C₁-C₆)alkyl, (C₁-C₆)alkylsulphonyloxy, (C₁-C₆)haloalkylsulphanyl, (C₃-C₆)cycloalkylsulphanyl, (C₃-C₆)halocycloalkylsulphanyl, (C₃-C₆)cycloalkylsulphonyl, (C₃-C₆)halocycloalkylsulphonyl, (C₃-C₆)cycloalkylsulphinyl, (C₃-C₆)halocycloalkylsulphinyl, (C₁-C₆)alkylcarbonyl, (C₁-C₆)haloalkylcarbonyl, (C₁-C₆)alkylcarbonyloxy, (C₁-C₆)alkoxycarbonyl, (C₁-C₆)haloalkoxycarbonyl, aminocarbonyl, (C₁-C₆)alkylaminocarbonyl, di-(C₁-C₆)alkylaminocarbonyl, (C₂-C₆)alkenylaminocarbonyl, di-(C₂-C₆)-alkenylaminocarbonyl, (C₃-C₈)cycloalkylaminocarbonyl, (C₁-C₆)alkylsulphonylamino, (C₁-C₆)alkylamino, di-(C₁-C₆)alkylamino, (C₁-C₆)haloalkylamino, bis-(C₁-C₆)haloalkylamino, aminosulphonyl, (C₁-C₆)alkylaminosulphonyl, di-(C₁-C₆)alkylaminosulphonyl, (C₁-C₆)alkylsulphoximino, aminothiocarbonyl, (C₁-C₆)alkylaminothiocarbonyl, di-(C₁-C₆)alkylaminothiocarbonyl, (C₃-C₈)cycloalkylamino,
R¹ is (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl, (C₃-C₈)cycloalkyl-(C₁-C₆)alkyl, (C₃-C₈)cycloalkyl-(C₃-C₈)cycloalkyl, (C₁-C₆)alkyl-(C₃-C₈)cycloalkyl, (C₁-C₆)haloalkyl-(C₃-C₈)cycloalkyl, halo(C₃-C₈)cycloalkyl, cyano(C₃-C₈)cycloalkyl, (C₂-C₆)alkynyl-(C₃-C₈)cycloalkyl, (C₁-C₆)alkoxy-(C₃-C₈)cycloalkyl, (C₁-C₆)alkoxycarbonyl-(C₃-C₈)cycloalkyl, (C₁-C₆)alkylamino, di-(C₁-C₆)alkylamino, (C₃-C₈)cycloalkylamino, (C₁-C₆)alkylcarbonylamino, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, (C₁-C₆)alkylsulphonylamino, aminosulphonyl-(C₁-C₆)alkyl, (C₁-C₆)alkylaminosulphonyl-(C₁-C₆)alkyl, di-(C₁-C₆)alkylaminosulphonyl-(C₁-C₆)alkyl, (C₁-C₄)alkoxy, (C₃-C₆)cycloalkoxy, (C₃-C₆)cycloalkylcarbonyl or (C₁-C₄)alkylcarbonyl,
or in each case optionally singly or multiply, identically or differently aryl-, hetaryl-, oxohetaryl-, heterocyclyl- or oxoheterocyclyl-substituted (C₁-C₆)alkyl, (C₁-C₆)alkoxy, (C₂-C₆)alkenyl, (C₂-C₆)alkynyl, (C₃-C₈)cycloalkyl, where aryl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, aminosulphonyl, (C₁-C₆)alkyl, (C₃-C₆)cycloalkyl, (C₁-C₆)alkoxy, (C₁-C₆)haloalkyl, (C₁-C₆)haloalkoxy, (C₁-C₆)alkylthio, (C₁-C₆)alkylsulphinyl, (C₁-C₆)alkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl or (C₃-C₆)trialkylsilyl,
or in each case optionally singly or multiply, identically or differently substituted aryl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₈)cycloalkyl, (C₁-C₆)-alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl,
R² is hydrogen (C₁-C₆)alkyl, (C₃-C₈)cycloalkyl, (C₁-C₆)alkoxy, (C₁-C₆)alkylcarbonyl, (C₃-C₈)cycloalkylcarbonyl, (C₁-C₆)alkoxycarbonyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl-(C₃-C₈)cycloalkyl, (C₁-C₆)alkyl-(C₃-C₈)cycloalkyl, halo(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, aminocarbonyl-(C₁-C₆)alkyl, (C₁-C₆)alkylamino-(C₁-C₆)alkyl, di-(C₁-C₆)alkylamino-(C₁-C₆)alkyl or (C₃-C₈)cycloalkylamino-(C₁-C₆)alkyl,
R³ is hydrogen (C₁-C₆)alkyl, (C₃-C₈)cycloalkyl, (C₁-C₆)alkoxy, (C₁-C₆)alkylcarbonyl, (C₃-C₈)cycloalkylcarbonyl, (C₁-C₆)alkoxycarbonyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl-(C₃-C₈)cycloalkyl, (C₁-C₆)alkyl-(C₃-C₈)cycloalkyl, halo(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkyl sulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, aminocarbonyl-(C₁-C₆)alkyl, (C₁-C₆)alkylamino-(C₁-C₆)alkyl, di-(C₁-C₆)alkylamino-(C₁-C₆)alkyl or (C₃-C₈)cycloalkylamino-(C₁-C₆)alkyl,
R⁴ is (C₁-C₆)alkyl, (C₁-C₆)haloalkyl, (C₁-C₆)cyanoalkyl, (C₁-C₆)hydroxyalkyl, (C₁-C₆)alkoxy-(C₁-C₆)alkyl, (C₁-C₆)haloalkoxy-(C₁-C₆)alkyl, (C₂-C₆)alkenyl, (C₂-C₆)alkenyloxy-(C₁-C₆)alkyl, (C₂-C₆)haloalkenyloxy-(C₁-C₆)alkyl, (C₂-C₆)haloalkenyl, (C₂-C₆)cyanoalkenyl, (C₂-C₆)alkynyl, (C₂-C₆)alkynyloxy-(C₁-C₆)alkyl, (C₂-C₆)haloalkynyloxy-(C₁-C₆)alkyl, (C₂-C₆)haloalkynyl, (C₂-C₆)cyanoalkynyl, (C₃-C₈)cycloalkyl, (C₃-C₈)cycloalkyl-(C₁-C₆)alkyl, cyano(C₃-C₈)cycloalkyl-(C₁-C₆)alkyl, halo(C₃-C₈)cycloalkyl-(C₁-C₆)alkyl, (C₁-C₄)haloalkyl-(C₃-C₈)cycloalkyl-(C₁-C₆)alkyl, (C₃-C₈)cycloalkyl-(C₃-C₈)cycloalkyl, (C₁-C₆)alkyl-(C₃-C₈)cycloalkyl, (C₁-C₆)haloalkyl-(C₃-C₈)cycloalkyl, halo(C₃-C₈)cycloalkyl, cyano(C₃-C₈)cycloalkyl, (C₂-C₆)alkynyl-(C₃-C₈)cycloalkyl, (C₁-C₆)alkoxy-(C₃-C₈)cycloalkyl, (C₁-C₆)alkoxycarbonyl-(C₃-C₈)cycloalkyl, carbamoyl-(C₃-C₈)cycloalkyl, thiocarbamoyl-(C₃-C₈)cycloalkyl, (C₁-C₆)alkylthio-(C₁-C₆)alkyl, (C₁-C₆)haloalkylthio-(C₁-C₆)alkyl, (C₁-C₆)alkylsulphinyl-(C₁-C₆)alkyl, (C₁-C₆)haloalkylsulphinyl-(C₁-C₆)alkyl, (C₁-C₆)alkylsulphonyl-(C₁-C₆)alkyl, (C₁-C₆)haloalkylsulphonyl-(C₁-C₆)alkyl, (C₁-C₆)alkoxy-(C₁-C₆)alkylthio-(C₁-C₆)alkyl, (C₁-C₆)alkoxy-(C₁-C₆)alkylsulphinyl-(C₁-C₆)alkyl, (C₁-C₆)alkoxy-(C₁-C₆)alkylsulphonyl-(C₁-C₆)alkyl, (C₁-C₆)alkylcarbonyl-(C₁-C₆)alkyl, (C₁-C₆)haloalkylcarbonyl-(C₁-C₆)alkyl, (C₁-C₆)alkoxycarbonyl-(C₁-C₆)alkyl, (C₁-C₆)haloalkoxycarbonyl-(C₁-C₆)alkyl, (C₁-C₆)alkylsulphonylamino, aminosulphonyl-(C₁-C₆)alkyl, (C₁-C₆)alkylaminosulphonyl-(C₁-C₆)alkyl, di-(C₁-C₆)alkylaminosulphonyl-(C₁-C₆)alkyl, amino, (C₁-C₆)alkylamino, di-(C₁-C₆)alkylamino, (C₃-C₈)cycloalkylamino, N—(C₁-C₆)alkyl-(C₃-C₈)cycloalkylamino, benzylamino, cyanobenzylamino, nitrobenzylamino, halobenzylamino, N—(C₁-C₆)alkylbenzylamino, N—(C₁-C₆)alkylcyanobenzylamino, N—(C₁-C₆)alkylnitrobenzylamino, N—(C₁-C₆)alkylhalobenzylamino, (C₁-C₆)alkylcarbonylamino, (C₃-C₈)cycloalkylcarbonylamino, hydroxyl, (C₁-C₆)alkoxy, (C₃-C₈)cycloalkoxy, (C₃-C₈)cycloalkyl-(C₁-C₆)alkoxy, cyano(C₁-C₆)alkoxy, benzyloxy, cyanobenzyloxy, nitrobenzyloxy, halobenzyloxy, (C₁-C₆)alkylimino, (C₃-C₈)cycloalkylimino, benzylimino, cyanobenzylimino, nitrobenzylimino, halobenzylimino, (C₁-C₆)haloalkylbenzylimino, halo-[(C₁-C₆)haloalkyl]benzylimino, (C₁-C₆)alkylcarbonyl, (C₁-C₆)haloalkylcarbonyl, (C₃-C₈)cycloalkylcarbonyl or (C₃-C₈)cycloalkyl-(C₁-C₆)alkylcarbonyl,
or in each case optionally singly or multiply, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₆)alkyl, (C₁-C₆)haloalkyl, (C₁-C₆)alkoxy, (C₂-C₆)alkenyl, (C₂-C₆)alkynyl, (C₃-C₈)cycloalkyl, oxy, amino, N—(C₁-C₆)alkylamino, N—(C₃-C₈)cycloalkylamino or carbonyl, where aryl, 1,3-benzodioxolyl, 2,3-dihydro-1,4-benzodioxinyl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, thiocarbamoyl, aminosulphonyl, (C₁-C₆)alkyl, (C₃-C₆)cycloalkyl, (C₁-C₆)alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)haloalkylsulphinyl, (C₁-C₆)haloalkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl or hetaryl, or
in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₈)cycloalkyl, (C₁-C₆)-alkoxy, (C₁-C₆)haloalkyl, (C₁-C₆)haloalkoxy, (C₁-C₆)alkylthio, (C₁-C₆)haloalkylthio, (C₁-C₆)alkylsulphinyl, (C₁-C₆)alkylsulphonyl, (C₁-C₆)haloalkylsulphinyl, (C₁-C₆)haloalkylsulphonyl, (C₁-C₆)alkylsulphimino, (C₁-C₆)alkylsulphimino-(C₁-C₆)alkyl, (C₁-C₆)alkylsulphimino-(C₂-C₆)alkylcarbonyl, (C₁-C₆)alkylsulphoximino, (C₁-C₆)alkylsulphoximino-(C₁-C₆)alkyl, (C₁-C₆)alkylsulphoximino-(C₂-C₆)alkylcarbonyl, (C₁-C₆)alkoxycarbonyl, (C₁-C₆)alkylcarbonyl, (C₃-C₆)trialkylsilyl,
or R³ and R⁴ together are (C₂-C₆)-alkyl or (C₂-C₆)alkenyl, forming a 3-7-membered, optionally halogen-, cyano-, hydroxyl-, amino-, carboxyl-, carbamoyl-, (C₁-C₆)alkyl-, (C₃-C₈)cycloalkyl-, (C₁-C₆)-alkoxy-, (C₁-C₆)haloalkyl-, (C₁-C₆)haloalkoxy-, (C₁-C₆)alkylthio- or (C₁-C₆)haloalkylthio-substituted ring which may optionally contain one to two double bonds,
R⁵, R⁶ are independently hydrogen, cyano, halogen, nitro, acetyl, hydroxyl, amino, (C₁-C₆)alkylamino, di-(C₁-C₆)alkylamino, (C₃-C₆)cycloalkyl, halo(C₃-C₆)cycloalkyl, (C₁-C₆)alkyl, (C₁-C₆)haloalkyl, (C₂-C₆)alkenyl, (C₂-C₆)haloalkenyl, (C₂-C₆)alkynyl, (C₂-C₆)haloalkynyl, (C₁-C₆)alkoxy, (C₁-C₆)haloalkoxy, (C₁-C₆)alkylthio, (C₁-C₆)haloalkylthio, (C₁-C₆)alkylsulphinyl, (C₁-C₆)haloalkylsulphinyl, (C₁-C₆)alkylsulphonyl, (C₁-C₆)haloalkylsulphonyl,
V¹ is oxygen or sulphur,
V² is oxygen, sulphur or —NH.

2. Compound of formula (I) and/or salt thereof according to claim 1 in which
Q¹ is in each case optionally singly or multiply, identically or differently substituted phenyl, 1,3-benzodioxolyl, 2,3-dihydro-1,4-benzodioxinyl, hetaryl or oxohetaryl,
where the substituents are selected from cyano, halogen, nitro, amino, SF₅, (C₁-C₄)alkyl, (C₁-C₄)haloalkyl, (C₁-C₄)cyanoalkyl, (C₁-C₄)hydroxyalkyl, (C₁-C₄)alkoxy-(C₁-C₄)alkyl, (C₂-C₄)alkenyl, (C₂-C₄)haloalkenyl, (C₂-C₄)cyanoalkenyl, (C₂-C₄)alkynyl, (C₂-C₄)haloalkynyl, (C₂-C₄)cyanoalkynyl, (C₁-C₄)alkoxy, (C₁-C₄)haloalkoxy, (C₁-C₄)cyanoalkoxy, (C₁-C₄)alkoxy-(C₁-C₄)alkoxy, (C₃-C₆)cycloalkyl, (C₃-C₆)halocycloalkyl, (C₁-C₄)alkyl-(C₃-C₆)cycloalkyl, (C₁-C₄)haloalkyl-(C₃-C₆)cycloalkyl, (C₁-C₄)haloalkenoxy, (C₁-C₄)alkylhydroxyimino, (C₁-C₄)alkoxyimino, (C₁-C₄)alkyl-(C₁-C₄)alkoxyimino, (C₁-C₄)haloalkyl-(C₁-C₄)alkoxyimino, (C₁-C₄)alkylsulphanyl, (C₁-C₄)alkylsulphanyl-(C₁-C₄)alkyl, (C₁-C₄)alkylsulphinyl, (C₁-C₄)haloalkylsulphinyl, (C₁-C₄)alkylsulphinyl-(C₁-C₄)alkyl, (C₁-C₄)alkylsulphonyl, (C₁-C₄)haloalkylsulphonyl, (C₁-C₄)alkylsulphonyl-(C₁-C₄)alkyl, (C₁-C₄)alkylsulphonyloxy, (C₁-C₄)haloalkylsulphanyl, (C₃-C₆)cycloalkylsulphanyl, (C₃-C₆)halocycloalkylsulphanyl, (C₃-C₆)cycloalkylsulphonyl, (C₃-C₆)halocycloalkylsulphonyl, (C₃-C₆)cycloalkylsulphinyl, (C₃-C₆)halocycloalkylsulphinyl, (C₁-C₄)alkylcarbonyl, (C₁-C₄)haloalkylcarbonyl, aminocarbonyl, (C₁-C₄)alkylaminocarbonyl, di-(C₁-C₄)alkylaminocarbonyl, (C₁-C₄)alkylsulphonylamino, (C₁-C₄)alkylamino, di-(C₁-C₄)alkylamino, (C₁-C₆)haloalkylamino, bis-(C₁-C₆)haloalkylamino, aminosulphonyl, (C₁-C₄)alkylaminosulphonyl, di-(C₁-C₄)alkylaminosulphonyl,
R¹ is (C₁-C₆)alkyl, (C₁-C₆)haloalkyl, (C₁-C₆)cyanoalkyl, (C₁-C₆)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₆)alkyl, (C₂-C₄)haloalkenyloxy-(C₁-C₆)alkyl, (C₂-C₄)haloalkenyl, (C₂-C₄)cyanoalkenyl, (C₂-C₄)alkynyl, (C₂-C₄)alkynyloxy-(C₁-C₆)alkyl, (C₂-C₄)haloalkynyloxy-(C₁-C₄)alkyl, (C₂-C₄)haloalkynyl, (C₂-C₄)cyanoalkynyl, (C₃-C₆)cycloalkyl, (C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, (C₁-C₆)alkylamino, di-(C₁-C₆)alkylamino, (C₃-C₆)cycloalkylamino, (C₁-C₄)alkylthio-(C₁-C₆)alkyl, (C₁-C₄)haloalkylthio-(C₁-C₆)alkyl, (C₁-C₄)alkylsulphinyl-(C₁-C₆)alkyl, (C₁-C₄)haloalkylsulphinyl-(C₁-C₆)alkyl, (C₁-C₄)alkylsulphonyl-(C₁-C₆)alkyl, (C₁-C₄)haloalkylsulphonyl-(C₁-C₆)alkyl, (C₁-C₄)alkylcarbonyl-(C₁-C₆)alkyl, (C₁-C₄)alkoxycarbonyl-(C₁-C₆)alkyl, (C₁-C₄)alkoxy, (C₃-C₆)cycloalkoxy, (C₃-C₆)cycloalkylcarbonyl or (C₁-C₄)alkylcarbonyl,
or is in each case optionally singly or multiply, identically or differently aryl-, hetaryl-, oxohetaryl-, heterocyclyl- or oxoheterocyclyl-substituted (C₁-C₄)alkyl, (C₁-C₄)alkoxy, (C₂-C₄)alkenyl, (C₂-C₄)alkynyl, (C₃-C₆)cycloalkyl, where aryl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)alkylsulphinyl, (C₁-C₄)alkylsulphonyl, (C₁-C₆)alkoxycarbonyl, (C₁-C₆)alkylcarbonyl or (C₃-C₆)trialkylsilyl,
or is in each case optionally singly or multiply, identically or differently substituted aryl, hetaryl, oxohetaryl, heterocyclyl or oxoheterocyclyl, where the substituents are selected from halogen, cyano, nitro, hydroxyl, amino, carboxyl, carbamoyl, (C₁-C₆)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₆)alkoxycarbonyl, (C₁-C₆)alkylcarbonyl, (C₃-C₆)trialkylsilyl,
R² is hydrogen, (C₁-C₆)alkyl, (C₃-C₆)cycloalkyl, (C₁-C₄)alkoxy, (C₁-C₆)alkylcarbonyl, (C₃-C₆)cycloalkylcarbonyl, (C₁-C₆)alkoxycarbonyl, (C₁-C₄)haloalkyl or (C₁-C₄)cyanoalkyl,
R³ is hydrogen, (C₁-C₆)alkyl, (C₃-C₆)cycloalkyl, (C₁-C₄)alkoxy, (C₁-C₆)alkylcarbonyl, (C₃-C₆)cycloalkylcarbonyl, (C₁-C₆)alkoxycarbonyl, (C₁-C₄)haloalkyl or (C₁-C₄)cyanoalkyl,
R⁴ is (C₁-C₆)alkyl, (C₁-C₆)haloalkyl, (C₁-C₆)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₆)alkenyl, (C₂-C₄)alkenyloxy-(C₁-C₆)alkyl, (C₂-C₄)haloalkenyloxy-(C₁-C₄)alkyl, (C₂-C₄)haloalkenyl, (C₂-C₆)cyanoalkenyl, (C₂-C₆)alkynyl, (C₂-C₄)alkynyloxy-(C₁-C₄)alkyl, (C₂-C₄)haloalkynyloxy-(C₁-C₄)alkyl, (C₂-C₆)haloalkynyl, (C₂-C₆)cyanoalkynyl, (C₃-C₆)cycloalkyl, (C₃-C₆)cycloalkyl-(C₁-C₆)alkyl, cyano(C₃-C₆)cycloalkyl-(C₁-C₆)alkyl, halo(C₃-C₆)cycloalkyl-(C₁-C₆)alkyl, (C₁-C₄)haloalkyl-(C₃-C₆)cycloalkyl-(C₁-C₆)alkyl, (C₃-C₆)cycloalkyl-(C₃-C₆)cycloalkyl, (C₁-C₆)alkyl-(C₃-C₆)cycloalkyl, (C₁-C₆)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, carbamoyl-(C₃-C₆)cycloalkyl, thiocarbamoyl-(C₃-C₆)cycloalkyl, (C₁-C₄)alkylthio-(C₁-C₄)alkyl, (C₁-C₄)haloalkylthio-(C₁-C₄)alkyl, (C₁-C₄)alkylsulphinyl-(C₁-C₄)alkyl, (C₁-C₄)haloalkylsulphinyl-(C₁-C₄)alkyl, (C₁-C₄)alkylsulphonyl-(C₁-C₄)alkyl, (C₁-C₄)haloalkylsulphonyl-(C₁-C₄)alkyl, (C₁-C₆)alkylcarbonyl-(C₁-C₆)alkyl, (C₁-C₆)haloalkylcarbonyl-(C₁-C₆)alkyl, (C₁-C₄)alkoxycarbonyl-(C₁-C₄)alkyl, (C₁-C₄)haloalkoxycarbonyl-(C₁-C₄)alkyl, (C₁-C₄)alkyl sulphonylamino, aminosulphonyl-(C₁-C₄)alkyl, (C₁-C₄)alkylaminosulphonyl-(C₁-C₄)alkyl, or di-(C₁-C₄)alkylaminosulphonyl-(C₁-C₆)alkyl, amino, (C₁-C₄)alkylamino, di-(C₁-C₄)alkylamino, (C₃-C₆)cycloalkylamino, N—(C₁-C₄)alkyl-(C₃-C₆)cycloalkylamino, benzylamino, cyanobenzylamino, nitrobenzylamino, halobenzylamino, N—(C₁-C₄)alkylbenzylamino, N—(C₁-C₄)alkylcyanobenzylamino, N—(C₁-C₄)alkylnitrobenzylamino, N—(C₁-C₄)alkylhalobenzylamino, (C₁-C₄)alkylcarbonylamino, (C₃-C₆)cycloalkylcarbonylamino, hydroxyl, (C₁-C₄)alkoxy, (C₃-C₆)cycloalkoxy, (C₃-C₆)cycloalkyl-(C₁-C₄)alkoxy, cyano(C₁-C₄)alkoxy, benzyloxy, cyanobenzyloxy, nitrobenzyloxy, halobenzyloxy, (C₁-C₄)alkylimino, (C₃-C₆)cycloalkylimino, benzylimino, cyanobenzylimino, nitrobenzylimino, halobenzylimino, (C₁-C₄)haloalkylbenzylimino, halo-[(C₁-C₄)haloalkyl]benzylimino, (C₁-C₄)alkylcarbonyl, (C₁-C₄)haloalkylcarbonyl, (C₃-C₆)cycloalkylcarbonyl or (C₃-C₆)cycloalkyl-(C₁-C₄)alkylcarbonyl,
or in each case optionally singly or multiply, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₄)alkyl, (C₁-C₄)haloalkyl, (C₁-C₄)alkoxy, (C₂-C₄)alkenyl, (C₂-C₄)alkynyl, (C₃-C₆)cycloalkyl, oxy, amino, N—(C₁-C₄)alkylamino, N—(C₃-C₆)cycloalkylamino or carbonyl, where aryl, 1,3-benzodioxolyl, 2,3-dihydro-1,4-benzodioxinyl, hetaryl, oxohetaryl, heterocyclyl or oxoheterocyclyl may each optionally be mono- or polysubstituted identically or differently by halogen, cyano, nitro, hydroxyl, amino, carboxyl, carbamoyl, thiocarbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkylsulphimino, (C₁-C₄)alkylsulphimino-(C₁-C₄)alkyl, (C₁-C₄)alkylsulphoximino, (C₁-C₄)alkylsulphoximino-(C₁-C₄)alkyl, (C₁-C₄)alkoxycarbonyl, (C₁-C₄)alkylcarbonyl, (C₃-C₆)trialkylsilyl or hetaryl, or
in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkylsulphimino, (C₁-C₄)alkylsulphimino-(C₁-C₄)alkyl, (C₁-C₄)alkylsulphoximino, (C₁-C₄)alkylsulphoximino-(C₁-C₄)alkyl, (C₁-C₄)alkoxycarbonyl, (C₁-C₄)alkylcarbonyl, (C₃-C₆)trialkylsilyl,
or R³ and R⁴ together are (C₂-C₆)-alkyl or (C₂-C₆)alkenyl, forming a 3-7-membered, optionally halogen-, cyano-, hydroxyl-, amino-, (C₁-C₄)alkyl-, (C₃-C₆)cycloalkyl-, (C₁-C₄)-alkoxy-, (C₁-C₄)haloalkyl-, (C₁-C₄)haloalkoxy-, (C₁-C₄)alkylthio- or (C₁-C₄)haloalkylthio-substituted ring which may optionally contain one to two double bonds,
R⁵, R⁶ are independently hydrogen, cyano, halogen, nitro, (C₃-C₆)cycloalkyl, (C₁-C₄)alkyl, (C₁-C₄)haloalkyl or (C₁-C₄)haloalkoxy,
V¹ is oxygen or sulphur,
V² is oxygen, sulphur or —NH.

3. Compound of formula (I) and/or salt thereof according to claim 1 in which
Q¹ is in each case optionally singly or multiply, identically or differently substituted phenyl, 1,3-benzodioxolyl, 2,3-dihydro-1,4-benzodioxinyl, pyrimidyl, pyridazinyl, pyridyl, thienyl, furanyl, oxazolyl, thiazolyl, imidazolyl or pyrazolyl,
where the substituents are selected from cyano, halogen, nitro, SF₅, (C₁-C₄)alkyl, (C₁-C₄)haloalkyl, (C₂-C₄)alkenyl, (C₂-C₄)haloalkenyl, (C₂-C₄)alkynyl, (C₂-C₄)haloalkynyl, (C₁-C₄)alkoxy, (C₁-C₄)haloalkoxy, (C₃-C₆)cycloalkyl, (C₃-C₆)halocycloalkyl, (C₁-C₄)alkyl-(C₃-C₆)cycloalkyl, (C₁-C₄)haloalkyl-(C₃-C₆)cycloalkyl, (C₁-C₄)haloalkenoxy, (C₁-C₄)alkylsulphanyl, (C₃-C₆)halocycloalkylsulphanyl, (C₁-C₄)alkylsulphinyl, (C₁-C₄)haloalkylsulphinyl, (C₃-C₆)cycloalkylsulphinyl, (C₁-C₄)alkylsulphonyl, (C₁-C₄)haloalkylsulphonyl, (C₃-C₆)cycloalkylsulphonyl, (C₁-C₄)haloalkylsulphanyl, (C₃-C₆)cycloalkylsulphanyl, (C₁-C₄)alkylcarbonyl, (C₁-C₄)haloalkylcarbonyl, bis-(C₁-C₄)alkylamino, di-(C₁-C₄)haloalkylamino,
R¹ is (C₁-C₄)alkyl, (C₁-C₄)haloalkyl, (C₁-C₄)hydroxyalkyl, (C₁-C₄)alkoxy-(C₁-C₄)alkyl, (C₂-C₄)alkenyl, (C₂-C₄)haloalkenyl, (C₂-C₄)alkynyl, (C₂-C₄)alkynyloxy-(C₁-C₄)alkyl, (C₃-C₆)cycloalkyl, (C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, (C₁-C₄)alkylthio-(C₁-C₆)alkyl, (C₁-C₄)alkylcarbonyl-(C₁-C₆)alkyl, (C₁-C₄)alkoxycarbonyl-(C₁-C₆)alkyl or (C₁-C₄)alkoxy,
or in each case singly or multiply, identically or differently aryl-, hetaryl- or heterocyclyl-substituted (C₁-C₄)alkyl, (C₃-C₆)cycloalkyl, where aryl, hetaryl or heterocyclyl may each optionally be mono- or polysubstituted identically or differently by halogen, cyano, nitro, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio or (C₁-C₆)alkoxycarbonyl,
or is in each case optionally singly or multiply, identically or differently substituted aryl, hetaryl, heterocyclyl or oxoheterocyclyl, where the substituents are selected from halogen, cyano, nitro, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₆)alkoxycarbonyl,
R² is hydrogen, (C₁-C₄)alkyl, (C₃-C₆)cycloalkyl, (C₁-C₄)alkoxy, (C₁-C₄)alkylcarbonyl, (C₃-C₆)cycloalkylcarbonyl or (C₁-C₄)alkoxycarbonyl,
R³ is hydrogen, (C₁-C₄)alkyl, (C₃-C₆)cycloalkyl, (C₁-C₄)alkoxy, (C₁-C₄)alkylcarbonyl, (C₃-C₆)cycloalkylcarbonyl or (C₁-C₄)alkoxycarbonyl,
R⁴ is (C₁-C₆)alkyl, (C₁-C₆)haloalkyl, (C₁-C₆)cyanoalkyl, (C₁-C₄)hydroxyalkyl, (C₁-C₄)alkoxy-(C₁-C₄)alkyl, (C₁-C₄)haloalkoxy-(C₁-C₄)alkyl, (C₂-C₄)alkenyl, (C₂-C₄)alkenyloxy-(C₁-C₄)alkyl, (C₂-C₄)haloalkenyloxy-(C₁-C₄)alkyl, (C₂-C₄)haloalkenyl, (C₂-C₄)cyanoalkenyl, (C₂-C₄)alkynyl, (C₂-C₄)alkynyloxy-(C₁-C₄)alkyl, (C₂-C₆)haloalkynyl, (C₂-C₆)cyanoalkynyl, (C₃-C₆)cycloalkyl, (C₃-C₆)cycloalkyl-(C₁-C₄)alkyl, cyano(C₃-C₆)cycloalkyl-(C₁-C₄)alkyl, halo(C₃-C₆)cycloalkyl-(C₁-C₄)alkyl, (C₁-C₄)haloalkyl-(C₃-C₆)cycloalkyl-(C₁-C₄)alkyl, (C₃-C₆)cycloalkyl-(C₃-C₆)cycloalkyl, (C₁-C₄)alkyl-(C₃-C₆)cycloalkyl, (C₁-C₄)haloalkyl-(C₃-C₆)cycloalkyl, halo(C₃-C₆)cycloalkyl, cyano(C₃-C₆)cycloalkyl, (C₂-C₄)alkynyl-(C₃-C₆)cycloalkyl, (C₁-C₄)alkoxy-(C₃-C₆)cycloalkyl, (C₁-C₄)alkoxycarbonyl-(C₃-C₆)cycloalkyl, carbamoyl-(C₃-C₆)cycloalkyl, thiocarbamoyl-(C₃-C₆)cycloalkyl, (C₁-C₄)alkylcarbonyl-(C₁-C₄)alkyl, (C₁-C₄)alkoxycarbonyl-(C₁-C₄)alkyl, amino, (C₁-C₄)alkylamino, di-(C₁-C₄)alkyl-amino, (C₃-C₆)cycloalkylamino, N—(C₁-C₄)alkyl-(C₃-C₆)cycloalkylamino, benzylamino, cyanobenzylamino, nitrobenzylamino, N—(C₁-C₄)alkylbenzylamino, N—(C₁-C₄)alkylcyanobenzylamino, N—(C₁-C₄)alkylnitrobenzylamino, (C₁-C₄)alkylcarbonylamino, (C₃-C₆)cycloalkylcarbonylamino, hydroxyl, (C₁-C₄)alkoxy, (C₃-C₆)cycloalkoxy, (C₃-C₆)cycloalkyl-(C₁-C₄)alkoxy, cyano(C₁-C₄)alkoxy, benzyloxy, cyanobenzyloxy, nitrobenzyloxy, (C₁-C₄)alkylimino, (C₃-C₆)cycloalkylimino, benzylimino, cyanobenzylimino, halobenzylimino, (C₁-C₄)haloalkylbenzylimino, halo-[(C₁-C₄)haloalkyl]benzylimino, nitrobenzylimino, (C₁-C₄)alkylsulphonyl-(C₁-C₄)alkyl, (C₁-C₄)alkylcarbonyl, (C₁-C₄)haloalkylcarbonyl, (C₃-C₆)cycloalkylcarbonyl or (C₃-C₆)cycloalkyl-(C₁-C₄)alkylcarbonyl,
or is in each case optionally singly or doubly, identically or differently aryl-, 1,3-benzodioxolyl-, 2,3-dihydro-1,4-benzodioxinyl-, hetaryl-, oxohetaryl-, heterocyclyl- or oxoheterocyclyl-substituted (C₁-C₄)alkyl, (C₁-C₄)haloalkyl, (C₂-C₄)alkenyl, (C₃-C₆)cycloalkyl, oxy, amino, N—(C₁-C₄)alkylamino, N-cyclopropylamino or carbonyl, where aryl, 1,3-benzodioxolyl, 2,3-dihydro-1,4-benzodioxinyl, hetaryl, oxohetaryl, heterocyclyl or oxoheterocyclyl may each optionally be singly or multiply, identically or differently substituted by halogen, cyano, nitro, hydroxyl, amino, carboxyl, carbamoyl, thiocarbamoyl, aminosulphonyl, (C₁-C₄)alkyl, (C₃-C₆)cycloalkyl, (C₁-C₄)alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkoxycarbonyl, (C₁-C₄)alkylcarbonyl, hetaryl, alkylhetaryl, heterocyclyl or oxoheterocyclyl,
or is in each case optionally singly or multiply, identically or differently substituted aryl, 2,3-dihydro-1H-indenyl, 1,3-benzodioxolyl, hetaryl, oxohetaryl, heterocyclyl or oxoheterocyclyl, where the substituents are selected from halogen, cyano, nitro, hydroxyl, amino, carboxyl, carbamoyl, (C₁-C₄)alkyl, (C₃-C₆)cycloalkyl, (C₁-C₄)-alkoxy, (C₁-C₄)haloalkyl, (C₁-C₄)haloalkoxy, (C₁-C₄)alkylthio, (C₁-C₄)haloalkylthio, (C₁-C₄)alkylsulphinyl, (C₁-C₄)alkylsulphonyl, (C₁-C₄)haloalkylsulphinyl, (C₁-C₄)haloalkylsulphonyl, (C₁-C₄)alkoxycarbonyl, (C₁-C₄)alkylcarbonyl, (C₃-C₆)trialkylsilyl,
or R³ and R⁴ together are (C₂-C₅)-alkyl or (C₂-C₅)alkenyl, forming a 3-6-membered, optionally halogen-, cyano-, (C₁-C₄)alkyl-, (C₃-C₆)cycloalkyl-, (C₁-C₄)-alkoxy-, (C₁-C₄)haloalkyl-, (C₁-C₄)haloalkoxy- or (C₁-C₄)haloalkylthio-substituted ring which may optionally contain one to two double bonds,
R⁵, R⁶ are independently hydrogen, cyano, halogen, methyl, ethyl, difluoromethyl, trifluoromethyl or trifluoromethoxy,
V¹ is oxygen or sulphur,
V² is oxygen, sulphur or —NH.

4. Compound of formula (I) and/or salt thereof according to claim 1 in which
Q¹ is in each case optionally singly or multiply, identically or differently substituted phenyl, 1,3-benzodioxolyl, 2,3-dihydro-1,4-benzodioxinyl, pyridyl, thienyl or pyrazol-1-yl or pyrazol-2-yl,
where the substituents are selected from cyano, fluorine, chlorine, bromine, iodine, SF₅, methyl, ethyl, n-propyl, i-propyl, cyclopropyl, fluoromethyl, difluoromethyl, trifluoromethyl, fluoroethyl, difluoroethyl, trifluoroethyl, tetrafluoroethyl, pentafluoroethyl, heptafluoroisopropyl, fluorocyclopropyl, difluorocyclopropyl, tetrafluorocyclopropyl, methoxy, ethoxy, trifluoromethoxy, difluoromethoxy, difluorochloromethoxy, dichlorofluoromethoxy, pentafluoroethoxy, tetrafluoroethoxy, trifluoroethoxy, chlorotetrafluoroethoxy, dichlorotrifluoroethoxy, trichlorodifluoroethoxy, chlorotrifluoroethoxy, chlorodifluoroethoxy, trifluoroethenoxy, trifluoromethylsulphonyl, difluoromethylsulphonyl, trifluoromethylsulphinyl, trifluoromethylsulphanyl, difluoromethylsulphinyl, difluoromethylsulphanyl, pentafluoroethylsulphanyl, trifluoroethylsulphanyl, difluoroethylsulphanyl, pentafluoroethylsulphonyl, tetrafluoroethylsulphonyl, trifluoroethylsulphonyl, difluoroethylsulphonyl, pentafluoroethylsulphinyl, tetrafluoroethylsulphinyl, trifluoroethylsulphinyl, difluoroethylsulphinyl, tetrafluoroethylsulphanyl, cyclopropylsulphanyl, trifluoromethylcyclopropyl, trifluoromethylcarbonyl, bis(trifluoromethyl)amino, (trifluorovinyl)oxy, heptafluororopoxy or hexafluoropropoxy,
R¹ is methyl, ethyl, n-propyl, i-propyl, cyclopropyl, methylcyclopropyl, cyclopropylmethyl, cyanocyclopropyl, chlorocyclopropyl, fluorocyclopropyl, trifluoromethylcyclopropyl, cyclopropylcyclopropyl, methoxymethyl, methoxyethyl, n-butyl, i-butyl, tert-butyl, cyclobutyl, difluorocyclobutyl, cyclopentyl, fluoromethyl, difluoromethyl, trifluoromethyl or trifluoroethyl,
or is mono-phenyl-substituted methyl, ethyl, i-propyl or cyclopropyl, where phenyl in each case is optionally mono- or disubstituted identically or differently by fluorine, chlorine, bromine, cyano, nitro, methyl, ethyl, n-propyl, i-propyl, cyclopropyl, trifluoromethyl, methoxy, trifluoromethoxy,
or is in each case optionally singly or doubly, identically or differently fluorine-, chlorine-, bromine-, cyano-, nitro-, methyl-, ethyl-, n-propyl-, i-propyl-, cyclopropyl-, trifluoromethyl-, methoxy-, trifluoromethoxy-substituted phenyl, pyridyl, oxazolyl, thiazolyl, thienyl, oxetanyl or thietanyl,
R² is hydrogen, methyl, ethyl, n-propyl, i-propyl, cyclopropyl, n-butyl, i-butyl, tert-butyl, cyclobutyl, methoxy, ethoxy, n-propoxy, i-propoxy, n-butoxy, i-butoxy, methylcarbonyl, ethylcarbonyl, n-propylcarbonyl, i-propylcarbonyl, cyclopropylcarbonyl, methoxycarbonyl, ethoxycarbonyl, n-propoxycarbonyl or i-propoxycarbonyl,
R³ is hydrogen, methyl, ethyl, n-propyl, i-propyl, cyclopropyl, n-butyl, i-butyl, tert-butyl, cyclobutyl, methoxy, ethoxy, n-propyloxy, i-propyloxy, n-butyloxy, i-butyloxy, methylcarbonyl, ethylcarbonyl, n-propylcarbonyl, i-propylcarbonyl, cyclopropylcarbonyl, methyloxycarbonyl, ethyloxycarbonyl, n-propyloxycarbonyl or i-propyloxycarbonyl,
R⁴ is methyl, ethyl, n-propyl, i-propyl, cyclopropyl, n-butyl, i-butyl, tert-butyl, cyclobutyl, fluoromethyl, difluoromethyl, trifluoromethyl, difluoroethyl, trifluoroethyl, pentafluoroethyl, trifluoropropyl, pentafluoropropyl, cyanomethyl, cyanoethyl, cyanopropyl, cyanobutyl, cyanopentyl, cyanohexyl, methyloxymethyl, ethyloxymethyl, methyloxyethyl, ethyloxyethyl, methyloxypropyl, ethyloxypropyl, propynyl, butynyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyclobutylethyl, cyclopentylethyl, cyclohexylethyl, cyanocyclopropylmethyl, cyanocyclobutylmethyl, cyanocyclopentylmethyl, cyanocyclohexylmethyl, trifluoromethylcyclopropylmethyl, trifluoromethylcyclohexylmethyl, cyclopropylcyclopropyl, methylcyclopropyl, ethylcyclopropyl, trifluoromethylcyclopropyl, trifluoromethylcyclobutyl, trifluoromethylcyclopentyl, trifluoromethylcyclohexyl, fluorocyclopropyl, difluorocyclopropyl, tetrafluorocyclopropyl, cyanocyclopropyl, cyanocyclobutyl, cyanocyclopentyl, cyanocyclohexyl, ethynylcyclopropyl, methyloxycyclopropyl, ethyloxycyclopropyl, methyloxycarbonylcyclopropyl, ethyloxycarbonylcyclopropyl, thiocarbamoylcyclopropyl, methyloxycarbonylmethyl, ethyloxycarbonylmethyl, methyloxycarbonylethyl, ethyloxycarbonylethyl, methylamino, ethylamino, n-propylamino, isopropylamino, cyclopropylamino, cyclobutylamino, cyclopentylamino, cyclohexylamino, benzylamino, cyanobenzylamino, nitrobenzylamino, dimethylamino, N-methylethylamino, N-methyl-n-propylamino, N-methyl-isopropylamino, N-methylcyclopropylamino, N-methylcyclobutylamino, N-methylcyclopentylamino, N-methylcyclohexylamino, N-methylbenzylamino, N-methylcyanobenzylamino, N-methylnitrobenzylamino, methoxy, ethoxy, n-propyloxy, isopropyloxy, cyclopropyloxy, cyclopropylmethyloxy, cyclobutyloxy, cyclopentyloxy, cyclohexyloxy, cyanomethyloxy, cyanoethyloxy, cyanopropyloxy, cyanobutyloxy, benzyloxy, cyanobenzyloxy, nitrobenzyloxy, methylimino, ethylimino, n-propylimino, isopropylimino, isobutylimino, cyclopentylimino, cyclohexylimino, benzylimino, cyanobenzylimino, nitrobenzylimino, fluorobenzylimino, chlorobenzylimino, trifluoromethylbenzylimino, chloro(trifluoromethyl)benzylimino, fluoro(trifluoromethyl)benzylimino, methyl sulphonylmethyl, methyl sulphonylethyl, methyl sulphonylpropyl, methyl sulphonylbutyl, methylcarbonyl, ethylcarbonyl, propylcarbonyl, isopropylcarbonyl, cyclopropylcarbonyl, cyclopropylmethylcarbonyl, cyclobutylmethylcarbonyl, butylcarbonyl, isobutylcarbonyl, tert-butylcarbonyl, cyclobutylcarbonyl, cyclopentylcarbonyl, cyclohexylcarbonyl or trifluoromethylcarbonyl,
or is mono-phenyl-, -pyridyl-, -pyrimidyl-, -furanyl-, -thiophenyl-, -oxazolyl-, -thiazolyl-, -indolyl-, -azaindolyl-, -indazolyl-, -azaindazolyl-, -1,3-benzodioxolyl- or -2,3-dihydro-1,4-benzodioxinyl-substituted methyl, ethyl, n-propyl, i-propyl, cyclopropyl, n-butyl, i-butyl, tert-butyl, cyclobutyl, cyclopentyl, cyclohexyl, oxy, amino, N-methylamino, N-ethylamino or carbonyl, where phenyl, pyridyl, pyrimidyl, furanyl, thiophenyl, oxazolyl, thiazolyl, indolyl, azaindolyl, indazolyl, azaindazolyl, 1,3-benzodioxolyl may each optionally be mono- or polysubstituted identically or differently by cyano, fluorine, chlorine, bromine, iodine, nitro, methyl, ethyl, n-propyl, i-propyl, cyclopropyl, trifluoromethyl, pentafluoroethyl, methoxy, ethoxy, trifluoromethoxy, pentafluoroethoxy, trifluoromethylthio, methyloxycarbonyl, ethyloxycarbonyl, carbamoyl, thiocarbamoyl, aminosulphonyl, pyrazolyl, imidazolyl, methylpyrazolyl, oxazolyl, oxdiazolyl, thiazolyl, pyrrolyl, pyrrolidinyl or pyrrolidinonyl,
or is in each case optionally mono- or disubstituted identically or differently by phenyl, pyridyl, pyrimidyl, thiophenyl, indazolyl, azaindazolyl, 1,3-benzodioxolyl, oxetanyl, thietanyl, tetrahydrofuranyl, oxotetrahydrofuranyl, tetrahydrothiophenyl, oxidotetrahydrothiophenyl, dioxidotetrahydrothiophenyl, tetrahydro-2H-pyranyl, oxotetrahydro-2H-pyranyl, tetrahydro-2H-thiopyranyl, oxotetrahydro-2H-thiopyranyl, 2,3-dihydro-1H-indenyl, where the substituents are selected from fluorine, chlorine, bromine, cyano, nitro, methyl, ethyl, n-propyl, i-propyl, cyclopropyl, trifluoromethyl, methoxy, trifluoromethoxy, trifluoromethylthio, methyloxycarbonyl or ethyloxycarbonyl,
or R³ and R⁴ together are (C₃-C₅)-alkyl, forming a 4-6-membered ring optionally substituted by halogen, cyano, trifluoromethyl or trifluoromethoxy,
R⁵, R⁶ are independently hydrogen, cyano, fluorine, chlorine, bromine, methyl, ethyl, difluoromethyl, trifluoromethyl or trifluoromethoxy,
V¹ is oxygen or sulphur,
V² is oxygen, sulphur or —NH.

5. Compound of formula (I) and/or salt thereof according to claim 1 in which
Q¹ is in each case optionally singly, doubly, triply or quadruply, identically or differently substituted phenyl, 1,3-benzodioxolyl, 2,3-dihydro-1,4-benzodioxinyl, pyridyl, thienyl or pyrazol-1-yl or pyrazol-2-yl,
where the substituents are selected from fluorine, chlorine, bromine, iodine, SF₅, methyl, ethyl, difluoromethyl, trifluoromethyl, difluoroethyl, trifluoroethyl, tetrafluoroethyl, pentafluoroethyl, heptafluoroisopropyl, trifluoromethoxy, difluoromethoxy, difluorochloromethoxy, pentafluoroethoxy, tetrafluoroethoxy, chlorotrifluoroethoxy, trifluoroethenoxy, trifluoromethylsulphonyl, trifluoromethylsulphinyl, trifluoromethylsulphanyl, difluoromethylsulphinyl, difluoromethylsulphanyl, trifluoromethylcyclopropyl, bis(trifluoromethyl)amino, tetrafluoroethylsulphanyl, cyclopropylsulphanyl, (trifluorovinyl)oxy and hexafluoropropoxy,
R¹ is methyl, ethyl, i-propyl, cyclopropyl, methylcyclopropyl, cyclopropylmethyl, cyanocyclopropyl, chlorocyclopropyl, cyclopropylcyclopropyl, methoxyethyl, tert-butyl, cyclobutyl, difluorocyclobutyl, cyclopentyl, trifluoroethyl or trifluoromethylcyclopropyl,
or mono-phenyl-substituted methyl, where phenyl may optionally be monosubstituted by chlorine, bromine, nitro, trifluoromethyl,
or is optionally mono-chlorine-substituted phenyl or pyridyl or is oxazolyl,
R² is hydrogen, methyl, ethyl, cyclopropylcarbonyl or methoxycarbonyl,
R³ is hydrogen, methyl, methoxy or methoxycarbonyl,
R⁴ is methyl, ethyl, n-propyl, i-propyl, cyclopropyl, n-butyl, i-butyl, cyclobutyl, trifluoroethyl, trifluoropropyl, cyanomethyl, cyanoethyl, cyanopropyl, cyanobutyl, cyanopentyl, methyloxyethyl, propynyl, butynyl, cyclopropyl, cyclobutyl, cyclopentyl, cyclohexyl, cyclopropylmethyl, cyclobutylmethyl, cyclopentylmethyl, cyclohexylmethyl, cyclopropylethyl, cyanocyclohexylmethyl, trifluoromethylcyclohexylmethyl, cyclopropylcyclopropyl, methylcyclopropyl, trifluoromethylcyclopropyl, trifluoromethylcyclohexyl, difluorocyclopropyl, cyanocyclopropyl, cyanocyclobutyl, cyanocyclohexyl, ethynylcyclopropyl, methyloxycyclopropyl, methyloxycarbonylcyclopropyl, thiocarbamoylcyclopropyl, methyloxycarbonylethyl, cyanobenzylimino, fluoro(trifluoromethyl)benzylimino, methyl sulphonylbutyl, methoxy, ethoxy, cyclopropylmethyloxy, isopropyloxy, cyanopropyloxy, cyanophenyloxy, cyclopropylcarbonyl,
or is mono-phenyl-, -pyridyl-, -indazolyl-, -azaindazolyl-, -thiophenyl- or -1,3-benzodioxolyl-substituted methyl, ethyl, n-propyl, cyclopropyl or carbonyl, where phenyl, pyridyl, indazolyl, azaindazolyl, thiophenyl or 1,3-benzodioxolyl may each optionally be mono-, di- or trisubstituted identically or differently by cyano, fluorine, chlorine, nitro, methyl, trifluoromethyl, methoxy, methyloxycarbonyl, thiocarbamoyl, aminosulphonyl, methylpyrazolyl, oxazolyl, oxadiazolyl or pyrrolidinonyl,
or is in each case optionally substituted phenyl, pyridyl, indazolyl, oxetanyl, thietanyl, tetrahydrofuranyl, oxotetrahydrofuranyl, dioxidotetrahydrothiophenyl, tetrahydro-2H-pyranyl or 2,3-dihydro-1H-indenyl, where the substituents are selected from chlorine, cyano, methyl and methyloxycarbonyl,
or R³ and R⁴ together are (C₃-C₅)-alkyl, forming a 4-6-membered ring optionally substituted by cyano,
R⁵, R⁶ are independently hydrogen,
V¹ is oxygen or sulphur,
V² is oxygen, sulphur or —NH.

6. Compound of formula (I) and/or salt thereof according to claim 1 in which
Q¹ is 2,2,3,3-tetrafluoro-2,3-dihydro-1,4-benzodioxin-6-yl, 2,2-difluoro-1,3-benzodioxol-5-yl, 2-chloro-4-(trifluoromethoxy)phenyl, 2-ethyl-6-methyl-4-(pentafluoroethyl)phenyl, 2-fluoro-4-(trifluoromethoxy)phenyl, 2-fluoro-6-(trifluoromethyl)pyridin-3-yl, 2-methyl-4-(trifluoromethoxy)phenyl, 3-(pentafluoroethyl)-1H-pyrazol-1-yl, 3-(trifluoromethoxy)phenyl, 3,5-difluoro-4-(trifluoromethoxy)phenyl, 3-chloro-4-(trifluoromethoxy)phenyl, 3-chloro-5-(trifluoromethyl)pyridin-2-yl, 3-fluoro-4-(pentafluoroethyl)phenyl, 3-fluoro-4-(trifluoromethoxy)phenyl, 3-fluoro-4-(trifluoromethyl)phenyl, 3-methyl-4-(trifluoromethoxy)phenyl, 4-(1,1,1,2,3,3,3-heptafluoropropan-2-yl)phenyl, 4-(1,1,2,2-tetrafluoroethoxy)phenyl, 4-(1,1-difluoroethyl)phenyl, 4-(2,2,2-trifluoroethyl)phenyl, 4-(2-chloro-1,1,2-trifluoroethoxy)phenyl, 4-(cyclopropylsulphanyl)phenyl, 4-(difluoromethoxy)-3,5-difluorophenyl, 4-(difluoromethoxy)phenyl, 4-(difluoromethyl)-3-fluorophenyl, 4-(pentafluoroethoxy)phenyl, 4-(pentafluoroethyl)phenyl, 4-SF₅-phenyl, 4-(trifluoromethoxy)phenyl, 4-(trifluoromethyl)phenyl, 4-[(1,1,2,2-tetrafluoroethyl)sulphanyl]phenyl, 4-[(difluoromethyl)sulphanyl]phenyl, 4-[(difluoromethyl)sulphinyl]phenyl, 4-[(trifluoromethyl)sulphanyl]phenyl, 4-[(trifluoromethyl)sulphinyl]phenyl, 4-[(trifluoromethyl)sulphonyl]phenyl, 4-[(trifluorovinyl)oxy]phenyl, 4-[1-(trifluoromethyl)cyclopropyl]phenyl, 4-[bis(trifluoromethyl)amino]phenyl, 4-[chloro(difluoro)methoxy]phenyl, 4-bromophenyl, 4-chlorophenyl, 4-fluorophenyl, 4-iodophenyl, 5-(trifluoromethyl)-2-thienyl, 5-(trifluoromethyl)pyridin-2-yl, 6-(trifluoromethyl)pyridin-3-yl, 4-bromo-3-fluorophenyl, 4-(1,1,2,3,3,3-hexafluoropropoxy)phenyl, 2,6-dichloro-4-(pentafluoroethyl)phenyl, 2-chloro-3-fluoro-4-(pentafluoroethyl)phenyl, 3-fluoro-5-methyl-4-(pentafluoroethyl)phenyl, 2,6-difluoro-4-(pentafluoroethyl)phenyl, 2-chloro-5-fluoro-4-(pentafluoroethyl)phenyl, 3-methyl-4-(pentafluoroethyl)phenyl, 3,5-difluoro-4-(pentafluoroethyl)phenyl, 2,3-difluoro-4-(pentafluoroethyl)phenyl, 3-chloro-4-(pentafluoroethyl)phenyl, 2,3,5-trifluoro-4-(pentafluoroethyl)phenyl, 2,5-difluoro-4-(pentafluoroethyl)phenyl, 2,6-dichloro-3-fluoro-4-(pentafluoroethyl)phenyl or 3-chloro-5-fluoro-4-(pentafluoroethyl)phenyl,
R¹ is cyclobutyl, cyclopentyl, cyclopropyl, cyclopropylmethyl, ethyl, isopropyl, methyl, tert-butyl, 1,3-oxazol-5-yl, 1-chlorocyclopropyl, 1-cyanocyclopropyl, 1-cyclopropylcyclopropyl, 1-methoxyethyl, 1-methylcyclopropyl, 2-chlorophenyl, 3,3-difluorocyclobutyl, 4-(trifluoromethyl)benzyl, 4-bromobenzyl, 4-chlorophenyl, 4-nitrobenzyl, 2,2,2-trifluoroethyl, 1-(trifluoromethyl)cyclopropyl or 6-chloropyridin-3-yl,
R² is hydrogen, methyl, ethyl, cyclopropylcarbonyl or methoxycarbonyl,
R³ is hydrogen, methyl, methoxy or methoxycarbonyl,
R⁴ is (1-methyl-1H-indazol-7-yl)methyl, (1-methyl-1H-pyrazolo[3,4-b]pyridin-5-yl)methyl, (1S)-1-[4-(trifluoromethyl)phenyl]ethyl, (1S)-1-[4-(trifluoromethyl)phenyl]ethyl, (2,2-difluoro-1,3-benzodioxol-5-yl)methyl, (4-cyanocyclohexyl)methyl, (5-cyanopyridin-2-yl)methyl, (6-chloropyridin-3-yl)methyl, (6-cyanopyridin-3-yl)methyl, (trans-4-cyanocyclohexyl)methyl, [cis-4-(trifluoromethyl)cyclohexyl]methyl, 1-(4-cyanophenyl)cyclopropyl, 1-(4-cyanophenyl)ethyl, 1-(4-nitrophenyl)cyclopropyl, 1-(4-nitrophenyl)ethyl, 1-(methoxycarbonyl)cyclopropyl, 1-(trifluoromethyl)cyclopropyl, 1,1-dioxidotetrahydrothiophen-3-yl, 1-[4-(trifluoromethyl)phenyl]ethyl, 1-thiocarbamoylcyclopropyl, 1-cyanocyclobutyl, 1-cyanocyclopropyl, 1-cyanoethyl, 1-cyclopropylcyclopropyl, 1-cyclopropylethyl, 1-ethynylcyclopropyl, 1-methoxycarbonylethyl, 1-methoxycyclopropyl, 1-methylcyclopropyl, 2-(3-cyanophenyl)ethyl, 2-(4-cyanophenyl)ethyl, 2,2,2-trifluoroethyl, 2,2-difluorocyclopropyl, 2,4-dichloro-5-fluorobenzyl, 2,4-dichlorobenzyl, 2-cyanopropan-2-yl, 2-fluoro-4-(trifluoromethyl)benzyl, 2-fluoro-4-nitrobenzyl, 2-methoxyethyl, 2-oxotetrahydrofuran-3-yl, 3-(methoxycarbonyl)oxetan-3-yl, 3,3,3-trifluoropropyl, 3-[4-(trifluoromethyl)phenyl]propyl, 3-chlorobenzyl, 3-cyanobenzyl, 3-cyanopentan-3-yl, 3-fluoro-4-(trifluorobenzyl)benzyl, 3-fluorobenzyl, 3-methylthietan-3-yl, 4-(1,2,4-oxadiazol-3-yl)benzyl, 4-(1,3,4-oxadiazol-2-yl)benzyl, 4-(1,3-oxazol-5-yl)benzyl, 4-(1-methyl-1H-pyrazol-3-yl)benzyl, 4-(1-methyl-1H-pyrazol-5-yl)benzyl, 4-(methoxycarbonyl)benzyl, 4-(trifluoromethyl)benzyl, 4-thiocarbamoylbenzyl, 4-chloro-2-fluorobenzyl, 4-chloro-3-fluorobenzyl, 4-chlorobenzyl, 4-chlorophenyl, 4-cyano-2,5-difluorobenzyl, 4-cyano-2,6-difluorobenzyl, 4-cyano-2-fluorobenzyl, 4-cyano-3,5-difluorobenzyl, 4-cyano-3-fluorobenzyl, 4-cyanobenzyl, 4-cyanobutyl, 4-cyanocyclohexyl, 4-cyanophenyl, 4-cyanotetrahydro-2H-pyran-4-yl, 4-fluoro-3-(trifluoromethyl)benzyl, 4-methoxybenzyl, 4-nitrobenzyl, 4-aminosulphonylbenzyl, 5-cyano-1H-indazol-3-yl, 5-cyano-2,3-dihydro-1H-inden-1-yl, 5-cyanopentyl, benzyl, but-3-yn-2-yl, n-butyl, cis-4-(trifluoromethyl)cyclohexyl, cyanomethyl, cyclobutyl, cyclobutylmethyl, cyclohexyl, cyclohexylmethyl, cyclopentyl, cyclopentylmethyl, cyclopropyl, cyclopropylmethyl, ethyl, isobutyl, isopropyl, methyl, oxetan-3-yl, prop-2-yn-1-yl, n-propyl, pyridin-2-ylmethyl, pyridin-3-yl, thietan-3-yl, trans-4-(trifluoromethyl)cyclohexyl, (5-nitro-2-thienyl)methyl, (5-cyano-2-thienyl)methyl, 4-cyanobenzylimine, 3-fluoro-4-(trifluoromethyl)benzylimine, [4-(2-oxopyrrolidin-1-yl)phenyl]methyl, 2-methyl-1-methylsulphonylpropan-2-yl, (pyridin-4-yl)methyl, methoxy, ethoxy, cyclopropylmethyloxy, isopropyloxy, 3-cyanopropyloxy, 4-cyanophenyloxy, cyclopropylcarbonyl or 4-nitrobenzoyl,
or R³ and R⁴ together are one of the following rings: 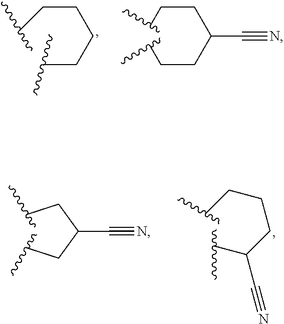 [SMILES: *CCCC* ; *CCC(C#N)CC* ; *CC(C#N)C* ; *CCCC(*)C#N]
R⁵, R⁶ are independently hydrogen,
V¹ is oxygen or sulphur,
V² is oxygen, sulphur or —NH.

7. Agrochemical formulation comprising a compound of formula (I) and/or salt thereof according to claim 1 in biologically effective content of 0.00000001% to 98% by weight, based on the weight of the agrochemical formulation, and one or more extenders and/or surfactants.

8. Agrochemical formulation according to claim 7, additionally comprising a further active agrochemical ingredient.

9. Method of controlling one or more animal pests comprising allowing a in which compound of formula (I) and/or salt thereof according to claim 1 to act on animal pests and/or a habitat thereof, excluding methods of treatment of a human or animal body.

10. A product comprising a compound of formula (I) and/or salt thereof according to claim 1 for control of animal pests, excluding treatment of a human or animal body.

11. A product comprising a compound of formula (I) and/or salt thereof according to claim 1 for protection of propagation material of plants.
